(19) United States
(12) Patent Application Publication
(10) Pub. No.: US 2025/0388682 A1
(43) Pub. Date: Dec. 25, 2025

(54) DEGRADATION OF EGFR USING A BISPECIFIC BINDING AGENT

(71) Applicant: EpiBiologics, Inc., San Mateo, CA (US)

(72) Inventors: Katarina PANCE, San Francisco, CA (US); Josef GRAMESPACHER, Seaside, CA (US); Rami HANNOUSH, San Mateo, CA (US); Hai TRAN, San Francisco, CA (US); Jonathan SITRIN, Brisbane, CA (US); Lisa MARSHALL, San Mateo, CA (US); Man-Tzu WANG, Foster City, CA (US); Kenneth NG, San Francisco, CA (US); Shyra GARDAI, San Mateo, CA (US)

(21) Appl. No.: 19/027,959

(22) Filed: Jan. 17, 2025

Related U.S. Application Data
(63) Continuation of application No. PCT/US2023/072125, filed on Aug. 11, 2023.
(60) Provisional application No. 63/371,371, filed on Aug. 12, 2022.
(60) Provisional application No. 63/384,877, filed on Nov. 23, 2022.
(60) Provisional application No. 63/479,497, filed on Jan. 11, 2023.

(51) Int. Cl. C07K 16/28; A61K 39/00; A61P 35/00; C07K 16/30; C07K 16/46

(52) U.S. Cl. CPC C07K 16/2863; A61P 35/00; C07K 16/3007; C07K 16/3092; C07K 16/468; A61K 2039/505

(57) ABSTRACT

The present disclosure provides methods of degrading an EGFR protein on a target cell. The present disclosure further discloses bispecific binding agents that bind to an EGFR protein and a membrane-associated internalizing protein.

### CROSS-REFERENCE

This application is a continuation of International Application No. PCT/US2023/072125, filed Aug. 11, 2023, which claims the benefit of U.S. Provisional Application No. 63/371,371, filed Aug. 12, 2022, U.S. Provisional Application No. 63/384,877, filed Nov. 23, 2022, and U.S. Provisional Application No. 63/479,497, filed Jan. 11, 2023, each of which is incorporated herein by reference in its entirety.

### SEQUENCE LISTING

The instant application contains a Sequence Listing which has been submitted electronically in XML format and is hereby incorporated by reference in its entirety. Said XML copy, created on Feb. 19, 2025, is named 6563-701_301_SL.xml and is 786,899 bytes in size, and is incorporated by reference as if written herein in its entirety.

### BACKGROUND

Targeted protein degradation is a promising new therapeutic strategy compared to conventional inhibition-based therapeutics. Inhibitors rely on sustained, occupancy-driven pharmacology, necessitating high affinity binders capable of abrogating catalytic or binding functions. Inhibiting protein-protein interactions or scaffolding functions has been extremely challenging for standard binding-based small molecules. In contrast, protein degraders are catalytic and utilize event-driven pharmacology, alleviating the need for high affinity binders, and durably abrogate all protein functions at once. As such, degrader technologies such as proteolysis targeting chimeras (PROTACs) have had great success in targeting traditionally challenging proteins. A number of PROTACs are currently in clinical trials.

Most degrader technologies, including PROTACs, utilize an intracellular mechanism of action and have thus been largely limited to targeting proteins with cytoplasmic domains. However, recent approaches, such as LYTACs have been described for specifically degrading cell surface proteins. These utilize recycling glycan receptors such as the mannose-6-phosphate receptor (M6PR) or asialoglycoprotein receptor (ASGR) to target proteins for internalization and trafficking to the lysosome for degradation. These require complex glycans conjugated to antibodies or to small molecules to effect degradation of a membrane protein.

As a hybrid approach that is broadly applicable to many cell types, we recently described antibody-based PROTACs (AbTACs). AbTACs utilize a standard IgG bispecific antibody format to bring a cell surface E3 ligase (RNF43) into proximity of a membrane protein of interest (POI) to mediate its degradation through the lysosomal pathway. The traditional bispecific IgG scaffold on which the AbTAC is built possesses favorable pharmacokinetic properties relative to LYTACS and other small molecule-based degraders. Furthermore, in contrast to other degradation modalities such as LYTACS and PROTACS, AbTACs are fully recombinant. However, there continues to exist a need for targeted protein degraders that efficiently and selectively induce the degradation of a target protein.

### SUMMARY

In an aspect, method of degrading a target protein on a surface of a target cell, the method comprising: contacting an endogenous internalizing receptor and the target protein on the surface of the target cell with a binding agent, wherein the binding agent comprises: (i) a first binding domain that specifically binds to an endogenous internalizing receptor, wherein the endogenous internalizing receptor is selected from the group consisting of MUC1, ITGB6, CEACAM5, and CDH17; (ii) a second binding domain that specifically binds to the target protein, wherein the target protein comprises EGFR.

In some embodiments, the binding agent is a multispecific antibody, a bispecific diabody, a bispecific Fab2, bispecific camelid antibody, a bispecific peptibody scFv-Fc, a bispecific IgG, a knob and hole bispecific IgG, a Fc-Fab, or a knob and hole bispecific Fc-Fab. In some embodiments, the first binding domain comprises a first binding domain variable heavy chain and a first binding domain variable light chain.

In some embodiments, the endogenous internalizing receptor is MUC1. In some embodiments, the first binding domain variable heavy chain comprises at least 80%, sequence identity to SEQ ID NO: 71. In some embodiments, the first binding domain variable heavy chain comprises at least 90%, sequence identity to SEQ ID NO: 71. In some embodiments, the first binding domain variable heavy chain comprises SEQ ID NO: 71. In some embodiments, the first binding domain variable light chain comprises at least 80% sequence identity to SEQ ID NO: 73.

In some embodiments, the first binding domain variable light chain comprises at least 90% sequence identity to SEQ ID NO: 73. In some embodiments, the first binding domain variable light chain comprises SEQ ID NO: 73. In some embodiments, the first binding domain binds to an epitope of the internalizing receptor on the target cell, wherein the epitope comprises at least 80% sequence identity to an epitope to which an antibody comprising SEQ ID NOs: 71 and 73 binds. In some embodiments, the first binding domain binds to an epitope of the internalizing receptor on the target cell, wherein the epitope comprises at least 90% sequence identity to the epitope to which the antibody comprising SEQ ID NOs: 71 and 73 binds. In some embodiments, the first binding domain binds to an epitope of the internalizing receptor on the target cell that does not include any of the amino acids from the epitope to which an antibody comprising SEQ ID NOs: 71 and 73 binds.

In some embodiments, the endogenous internalizing receptor is CDH17. In some embodiments, the first binding domain variable heavy chain comprises at least 80%, sequence identity to SEQ ID NO: 47. In some embodiments, the first binding domain variable heavy chain comprises at least 90%, sequence identity to SEQ ID NO: 47. In some embodiments, the first binding domain variable heavy chain comprises SEQ ID NO: 47. In some embodiments, the first binding domain variable light chain comprises at least 80% sequence identity to SEQ ID NO: 49. In some embodiments, the first binding domain variable light chain comprises at least 90% sequence identity to SEQ ID NO: 49. In some embodiments, the first binding domain variable light chain comprises SEQ ID NO: 49.

In some embodiments, the first binding domain binds to an epitope of the internalizing receptor on the target cell, wherein the epitope comprises at least 80% sequence identity to an epitope to which an antibody comprising SEQ ID NOs: 47 and 49 binds. In some embodiments, the first binding domain binds to an epitope of the internalizing receptor on the target cell, wherein the epitope comprises at least 90% sequence identity to the epitope to which the antibody comprising SEQ ID NOs: 47 and 49 binds. In some embodiments, the first binding domain binds to an epitope of the internalizing receptor on the target cell that does not include any of the amino acids from the epitope to which the antibody comprising SEQ ID NOs: 47 and 49 binds.

In some embodiments, the endogenous internalizing receptor is ITGB6. In some embodiments, the first binding domain variable heavy chain comprises at least 80%, sequence identity to SEQ ID NO: 287. In some embodiments, the first binding domain variable heavy chain comprises at least 90%, sequence identity to SEQ ID NO: 287. In some embodiments, the first binding domain variable heavy chain comprises SEQ ID NO: 287. In some embodiments, the first binding domain variable light chain comprises at least 80% sequence identity to SEQ ID NO: 289. In some embodiments, the first binding domain variable light chain comprises at least 90% sequence identity to SEQ ID NO: 289. In some embodiments, the first binding domain variable light chain comprises SEQ ID NO: 289.

In some embodiments, the first binding domain binds to an epitope of the internalizing receptor on the target cell, wherein the epitope comprises at least 80% sequence identity to an epitope to which an antibody comprising SEQ ID NOs: 287 and 289 binds. In some embodiments, the first binding domain binds to an epitope e of the internalizing receptor on the target cell, wherein the epitope comprises at least 90% sequence identity to the epitope to which the antibody comprising SEQ ID NOs: 287 and 289 binds. In some embodiments, the first binding domain binds to an epitope of the internalizing receptor on the target cell that does not include any of the amino acids from the epitope to which the antibody comprising SEQ ID NOs: 287 and 289 binds.

In some embodiments, the endogenous internalizing receptor is CEACAM5. In some embodiments, the first binding domain variable heavy chain comprises at least 80%, sequence identity to SEQ ID NO: 87. In some embodiments, the first binding domain variable heavy chain comprises at least 90%, sequence identity to SEQ ID NO: 87. In some embodiments, the first binding domain variable heavy chain comprises SEQ ID NO: 87. In some embodiments, the first binding domain variable light chain comprises at least 80% sequence identity to SEQ ID NO: 89. In some embodiments, the first binding domain variable light chain comprises at least 90% sequence identity to SEQ ID NO: 89. In some embodiments, the first binding domain variable light chain comprises SEQ ID NO: 89.

In some embodiments, the first binding domain binds to an epitope of the internalizing receptor on the target cell, wherein the epitope comprises at least 80% sequence identity to an epitope to which an antibody comprising SEQ ID NOs: 87 and 89 binds. In some embodiments, the first binding domain binds to an epitope of the internalizing receptor on the target cell, wherein the epitope comprises at least 90% sequence identity to the epitope to which the antibody comprising SEQ ID NOs: 87 and 89 binds. In some embodiments, the first binding domain binds to an epitope of the internalizing receptor on the target cell that does not include any of the amino acids from the epitope to which the antibody comprising SEQ ID NOs: 87 and 89 binds.

In some embodiments, the second binding domain comprises a second binding domain variable heavy chain. In some embodiments, the second binding domain variable heavy chain comprises at least 80%, sequence identity to SEQ ID NO: 651. In some embodiments, the second binding domain variable heavy chain comprises at least 90%, sequence identity to SEQ ID NO: 651. In some embodiments, the second binding domain variable heavy chain comprises SEQ ID NO: 651.

In some embodiments, the second binding domain binds to an epitope of the target protein on the target cell, wherein the epitope comprises at least 80% sequence identity to an epitope to which Cetuximab binds. In some embodiments, the second binding domain binds to an epitope of the target protein on the target cell, wherein the epitope comprises at least 90% sequence identity to the epitope to which Cetuximab binds. In some embodiments, the second binding domain binds to an epitope of the target protein on the target cell that does not include any of the amino acids from the epitope to which Cetuximab binds.

In some embodiments, the second binding domain binds to an epitope of the target protein on the target cell, wherein the epitope comprises at least 80% sequence identity to an epitope to which Mav2 binds. In some embodiments, the second binding domain binds to an epitope of the target protein on the target cell, wherein the epitope comprises at least 90% sequence identity to the epitope to which Mav2 binds. In some embodiments, the second binding domain binds to an epitope of the target protein on the target cell that does not include any of the amino acids from the epitope to which Mav2 binds.

In some embodiments, following the contacting, EGFR is internalized with the endogenous internalizing receptor into the target cell and EGFR is degraded. In some embodiments, wherein the endogenous internalizing receptor is recycled to the target cell surface following the internalization of the binding agent. In some embodiments, the endogenous internalizing receptor is degraded.

In some embodiments, the target cell is a cancer cell. In some embodiments, the cancer cell is selected from the group consisting of a breast cancer cell, a B cell lymphoma cell, a pancreatic cancer cell, a Hodgkin's lymphoma cell, an ovarian cancer cell, a prostate cancer cell, a mesothelioma cell, a lung cancer cell, a non-Hodgkin's B-cell (B-NHL) cell, a melanoma cell, a chronic lymphocytic leukemia cell, an acute lymphocytic leukemia cell, a neuroblastoma cell, a glioma cell, a glioblastoma cell, a bladder cancer cell, a colorectal cancer cell, and head and neck cancer cell.

In some embodiments, expression of EGFR on the cancer cell decreases following contact with the multispecific binding agent, as compared to a control cancer cell that is not contacted with the binding agent. In some embodiments, expression of EGFR on the cancer cell decreases by 50% or more relative to expression of EGFR on the control cancer cell not contacted with the binding agent. In some embodiments, expression of EGFR on the cancer cell decreases by 50% or more relative to expression of EGFR on the control cancer cell contacted with a monospecific EGFR binding agent. In some embodiments, cell surface removal of EGFR on the cancer cell is at least 20% or more relative to EGFR on the control cancer cell not contacted with the binding agent. In some embodiments, cell surface removal of EGFR on the cancer cell is at least 20% or more relative to EGFR on the control cancer cell contacted with a monospecific EGFR binding agent.

In some embodiments, internalization of EGFR in the cancer cell is at least 20% or more relative to internalizing of EGFR in the control cancer cell not contacted with the binding agent. In some embodiments, internalization of EGFR on the cancer cell is at least 20% or more relative to EGFR on the control cancer cell contacted with a monospecific EGFR binding agent. In some embodiments, degradation of EGFR in the cancer cell is at least 20% or more relative to degradation of EGFR in the control cancer cell not contacted with the binding agent. In some embodiments, cell degradation of EGFR on the cancer cell is at least 20% or more relative to EGFR on the control cancer cell contacted with a monospecific EGFR binding agent. In some embodiments, the monospecific EGFR binding agent is Cetuximab.

In some embodiments, the method increases the susceptibility of the cancer cell to cancer therapeutic agents. In some embodiments, the cancer therapeutic agent is a cytotoxic agent. In some embodiments, the method reduces proliferation of the cancer cell. In some embodiments, the method increases death of the cancer cell. In some embodiments, the contacting is performed in vivo.

In another aspect, the present disclosure provides a method for treating cancer in a subject, the method comprising: administering to a subject a binding agent, wherein the binding agent comprises: (i) a first binding domain that specifically binds to an endogenous internalizing receptor, wherein the endogenous internalizing receptor is expressed on a target cell, and wherein the endogenous internalizing receptor is selected from the group consisting of MUC1, ITGB6, CEACAM5, and CDH17; (ii) a second binding domain that specifically binds to the target protein, wherein the target protein comprises EGFR.

In some embodiments, the endogenous internalizing receptor is MUC1. In some embodiments, the endogenous internalizing receptor is ITGB6. In some embodiments, the endogenous internalizing receptor is CEACAM5. In some embodiments, the endogenous internalizing receptor is CDH17.

In some embodiments, the cancer is breast cancer, B cell lymphoma, pancreatic cancer, Hodgkin's lymphoma, ovarian cancer, prostate cancer, mesothelioma, lung cancer, non-Hodgkin's B-cell (B-NHL) lymphoma, melanoma, chronic lymphocytic leukemia, acute lymphocytic leukemia, neuroblastoma, glioma, glioblastoma, bladder cancer, colorectal cancer, or head and neck cancer.

In some embodiments, tumor volume of the tumor contacted with the multispecific binding agent decreases by 20% or more relative to the tumor volume of a tumor not contacted with the bispecific binding agent. In some embodiments, tumor volume of the tumor contacted with the multispecific binding agent is at least 80% or less in volume relative to the tumor volume of a tumor not contacted with the bispecific binding agent. In some embodiments, expression of EGFR on the cancer cell decreases by 20% relative to the EGFR expression of a cancer cell not contacted with the bispecific binding agent. In some embodiments, expression of EGFR on the cancer cell decreases by 20% relative to the EGFR expression of a cancer cell contacted with a monospecific EGFR binding agent. In some embodiments, the monospecific EGFR binding agent is Cetuximab.

In another aspect, the present disclosure provides a multispecific binding agent comprising: (a) a first binding domain that specifically binds to an endogenous internalizing receptor, wherein the endogenous internalizing receptor is selected from a group consisting of MUC1, ITGB6, CEACAM5, or CDH17; and (b) a second binding domain that specifically binds to a target protein, wherein the target protein is EGFR.

In some embodiments, the multispecific binding agent is a multispecific antibody, bispecific antibody, a bispecific diabody, a bispecific Fab2, bispecific camelid antibody, a bispecific peptibody scFv-Fc, a bispecific IgG, a knob and hole bispecific IgG, a Fc-Fab, or a knob and hole bispecific Fc-Fab. In some embodiments, the first binding domain comprises a first binding domain variable heavy chain and a first binding domain variable light chain.

In some embodiments, the endogenous internalizing receptor is MUC1. In some embodiments, the first binding domain variable heavy chain comprises at least 80% sequence identity to SEQ ID NO: 71. In some embodiments, the first binding domain variable heavy chain comprises at least 90% sequence identity to SEQ ID NO: 71. In some embodiments, the first binding domain variable heavy chain comprises SEQ ID NO: 71. In some embodiments, the first binding domain variable light chain comprises at least 80% sequence identity to SEQ ID NO: 73. In some embodiments, the first binding domain variable light chain comprises at least 90% sequence identity to SEQ ID NO: 73. In some embodiments, the first binding domain variable light chain comprises SEQ ID NO: 73.

In some embodiments, the first binding domain binds to an epitope of the internalizing receptor on the target cell, wherein the epitope comprises at least 80% sequence identity to an epitope to which an antibody comprising SEQ ID NOs: 71 and 73 binds. In some embodiments, the first binding domain binds to an epitope of the internalizing receptor on the target cell, wherein the epitope comprises at least 90% sequence identity to an epitope to which the antibody comprising SEQ ID NOs: 71 and 73 binds. In some embodiments, the first binding domain binds to an epitope of the internalizing receptor on the target cell that does not include any of the amino acids from the epitope to which the antibody comprising SEQ ID NOs: 71 and 73 binds.

In some embodiments, the endogenous internalizing receptor is ITGB6. In some embodiments, the first binding domain variable heavy chain comprises at least 80% sequence identity to SEQ ID NO: 287. In some embodiments, the first binding domain variable heavy chain comprises at least 90% sequence identity to SEQ ID NO: 287. In some embodiments, the first binding domain variable heavy chain comprises SEQ ID NO: 287. In some embodiments, the first binding domain variable light chain comprises at least 80% sequence identity to SEQ ID NO: 289. In some embodiments, the first binding domain variable light chain comprises at least 90% sequence identity to SEQ ID NO: 289. In some embodiments, the first binding domain variable light chain comprises SEQ ID NO: 289.

In some embodiments, the first binding domain binds to an epitope of the internalizing receptor on the target cell, wherein the epitope comprises at least 80% sequence identity to an epitope to which an antibody comprising SEQ ID NOs: 287 and 289 binds. In some embodiments, the first binding domain binds to an epitope of the internalizing receptor on the target cell, wherein the epitope comprises at least 90% sequence identity to an epitope to which the antibody comprising SEQ ID NOs: 287 and 289 binds. In some embodiments, the first binding domain binds to an epitope of the internalizing receptor on the target cell that does not include any of the amino acids from the epitope to which the antibody comprising SEQ ID NOs: 287 and 289 binds.

In some embodiments, the endogenous internalizing receptor is CEACAM5. In some embodiments, the first binding domain variable heavy chain comprises at least 80% sequence identity to SEQ ID NO: 87. In some embodiments, the first binding domain variable heavy chain comprises at least 90% sequence identity to SEQ ID NO: 87. In some embodiments, the first binding domain variable heavy chain comprises SEQ ID NO: 87. In some embodiments, the first binding domain variable light chain comprises at least 80% sequence identity to SEQ ID NO: 89. In some embodiments, the first binding domain variable light chain comprises at least 90% sequence identity to SEQ ID NO: 89. In some embodiments, the first binding domain variable light chain comprises SEQ ID NO: 89.

In some embodiments, the first binding domain binds to an epitope of the internalizing receptor on the target cell, wherein the epitope comprises at least 80% sequence identity to an epitope to which an antibody comprising SEQ ID NOs: 87 and 89 binds. In some embodiments, the first binding domain binds to an epitope of the internalizing receptor on the target cell, wherein the epitope comprises at least 90% sequence identity to an epitope to which the antibody comprising SEQ ID NOs: 87 and 89 binds. In some embodiments, the first binding domain binds to an epitope of the internalizing receptor on the target cell that does not include any of the amino acids from the epitope to which the antibody comprising SEQ ID NOs: 87 and 89 binds.

In some embodiments, the endogenous internalizing receptor is CDH17. In some embodiments, the first binding domain variable heavy chain comprises at least 80% sequence identity to SEQ ID NO: 47. In some embodiments, the first binding domain variable heavy chain comprises at least 90% sequence identity to SEQ ID NO: 47. In some embodiments, the first binding domain variable heavy chain comprises SEQ ID NO: 47. In some embodiments, the first binding domain variable light chain comprises at least 80% sequence identity to SEQ ID NO: 49. In some embodiments, the first binding domain variable light chain comprises at least 90% sequence identity to SEQ ID NO: 49. In some embodiments, the first binding domain variable light chain comprises SEQ ID NO: 49.

In some embodiments, the first binding domain binds to an epitope of the internalizing receptor on the target cell, wherein the epitope comprises at least 80% sequence identity to an epitope to which an antibody comprising SEQ ID NOs: 47 and 49 binds. In some embodiments, the first binding domain binds to an epitope of the internalizing receptor on the target cell, wherein the epitope comprises at least 90% sequence identity to an epitope to which an antibody comprising SEQ ID NOs: 47 and 49 binds. In some embodiments, the first binding domain binds to an epitope of the internalizing receptor on the target cell that does not include any of the amino acids from the epitope to which an antibody comprising SEQ ID NOs: 47 and 49 binds.

In some embodiments, the second binding domain comprises a second binding domain variable heavy chain. In some embodiments, the second binding domain variable heavy chain comprises at least 80% sequence identity to SEQ ID NO: 651. In some embodiments, the second binding domain variable heavy chain comprises at least 90% sequence identity to SEQ ID NO: 651. In some embodiments, the second binding domain variable heavy chain comprises SEQ ID NO: 651.

In some embodiments, the second binding domain binds to an epitope of the target protein on the target cell, wherein the epitope comprises at least 80% sequence identity to an epitope to which Cetuximab binds. In some embodiments, the second binding domain binds to an epitope of the target protein on the target cell, wherein the epitope comprises at least 90% sequence identity to the epitope to which Cetuximab binds. In some embodiments, the second binding domain binds to an epitope of the target protein on the target cell that does not include any of the amino acids from the epitope to which Cetuximab binds.

In some embodiments, the second binding domain binds to an epitope of the target protein on the target cell, wherein the epitope comprises at least 80% sequence identity to an epitope to which Mav2 binds. In some embodiments, the second binding domain binds to an epitope of the target protein on the target cell, wherein the epitope comprises at least 90% sequence identity to the epitope to which Mav2 binds. In some embodiments, the second binding domain binds to an epitope of the target protein on the target cell that does not include any of the amino acids from the epitope to which Mav2 binds.

In some embodiments, the half-life of the multispecific binding agent is within 20% of the half-life of Cetuximab. In some embodiments, the clearance rate of the multispecific binding agent is within 20-95% of the clearance rate of Cetuximab. In some embodiments, the Kd of the multispecific binding agent is within two-fold of the binding affinity of Cetuximab to EGFR. In some embodiments, the multispecific binding agent is within five-fold of the binding affinity of Cetuximab to EGFR. In some embodiments, the multispecific binding agent is within ten-fold of the binding affinity of Cetuximab to EGFR. In some embodiments, the Kd of the binding affinity of the multispecific binding agent may be within an order of magnitude of the binding affinity of a monovalent binding agent.

In some embodiments, the Kd of the multispecific binding agent is within +/−10% of the binding affinity of Cetuximab to EGFR. In some embodiments, the Kd of the multispecific binding agent is within +/−20% of the binding affinity of Cetuximab to EGFR. In some embodiments, the Kd of the multispecific binding agent is within +/−30% of the binding affinity of Cetuximab to EGFR. In some embodiments, the Kd of the multispecific binding agent is less than the binding affinity of Cetuximab to EGFR. In some embodiments, the Kd of the multispecific binding agent is more than the binding affinity of Cetuximab to EGFR.

In yet another aspect, the present disclosure provides a method of degrading a target protein on a surface of a target cell, the method comprising: contacting an endogenous internalizing receptor and the target protein on the surface of the target cell with a binding agent, wherein the binding agent comprises: (i) a first binding domain that specifically binds to an endogenous internalizing receptor, wherein the endogenous internalizing receptor comprises B7-H3; (ii) a second binding domain that specifically binds to the target protein, wherein the target protein comprises EGFR.

In some embodiments, the binding agent is a multispecific antibody, a bispecific diabody, a bispecific Fab2, bispecific camelid antibody, a bispecific peptibody scFv-Fc, a bispecific IgG, a knob and hole bispecific IgG, a Fc-Fab, or a knob and hole bispecific Fc-Fab. In some embodiments, the first binding domain binds to an epitope of the endogenous internalizing receptor on the target cell, wherein the epitope comprises at least 80% sequence identity to an epitope to which an antibody comprising SEQ ID NOs: 99 and 101 binds. In some embodiments, the first binding domain binds to an epitope of the endogenous internalizing receptor on the target cell, wherein the epitope comprises at least 90% sequence identity to the epitope to which the antibody comprising SEQ ID NOs: 99 and 101 binds. In some embodiments, the first binding domain binds to an epitope of the endogenous internalizing receptor on the target cell that does not include any of the amino acids from the epitope to which the antibody comprising SEQ ID NOs: 99 and 101 binds.

In some embodiments, the second binding domain binds to an epitope of the target protein on the target cell, wherein the epitope comprises at least 80% sequence identity to an epitope to which Cetuximab binds. In some embodiments, the second binding domain binds to an epitope of the target protein on the target cell, wherein the epitope comprises at least 90% sequence identity to the epitope to which Cetuximab binds. In some embodiments, the second binding domain binds to an epitope of the target protein on the target cell that does not include any of the amino acids from the epitope to which Cetuximab binds.

In some embodiments, the second binding domain binds to an epitope of the target protein on the target cell, wherein the epitope comprises at least 80% sequence identity to an epitope to which Mav2 binds. In some embodiments, the second binding domain binds to an epitope of the target protein on the target cell, wherein the epitope comprises at least 90% sequence identity to the epitope to which Mav2 binds. In some embodiments, the second binding domain binds to an epitope of the target protein on the target cell that does not include any of the amino acids from the epitope to which Mav2 binds.

In some embodiments, the first binding domain comprises a first binding domain variable heavy chain and a first binding domain variable light chain. In some embodiments, the first binding domain variable heavy chain comprises at least 80%, sequence identity to SEQ ID NO: 99. In some embodiments, the first binding domain variable heavy chain comprises at least 90%, sequence identity to SEQ ID NO: 99. In some embodiments, the first binding domain variable heavy chain comprises SEQ ID NO: 99. In some embodiments, the first binding domain variable light chain comprises at least 80% sequence identity to SEQ ID NO: 101. In some embodiments, the first binding domain variable light chain comprises at least 90% sequence identity to SEQ ID NO: 101. In some embodiments, the first binding domain variable light chain comprises SEQ ID NO: 101.

In some embodiments, the second binding domain comprises a second binding domain variable heavy chain. In some embodiments, the second binding domain variable heavy chain comprises at least 80% sequence identity to SEQ ID NO: 655. In some embodiments, the second binding domain variable heavy chain comprises at least 90% sequence identity to SEQ ID NO: 655. In some embodiments, the second binding domain variable heavy chain comprises SEQ ID NO: 655.

In some embodiments, the endogenous internalizing receptor is recycled to the target cell surface following the internalization of the binding agent. In some embodiments, the endogenous internalizing receptor is degraded. In some embodiments, the target cell is a cancer cell. In some embodiments, the cancer cell is selected from the group consisting of a breast cancer cell, a B cell lymphoma cell, a pancreatic cancer cell, a Hodgkin's lymphoma cell, an ovarian cancer cell, a prostate cancer cell, a mesothelioma cell, a lung cancer cell, a non-Hodgkin's B-cell (B-NHL) cell, a melanoma cell, a chronic lymphocytic leukemia cell, an acute lymphocytic leukemia cell, a neuroblastoma cell, a glioma cell, a glioblastoma cell, a bladder cancer cell, a colorectal cancer cell, and head and neck cancer cell.

In some embodiments, expression of EGFR on the cancer cell decreases following contact with the multispecific binding agent, as compared to a control cancer cell that is not contacted with the binding agent. In some embodiments, expression of EGFR on the cancer cell decreases by 50% or more relative to expression of EGFR on the control cancer cell not contacted with the binding agent. In some embodiments, expression of EGFR on the cancer cell decreases by 50% or more relative to expression of EGFR on the control cancer cell contacted with a monospecific EGFR binding agent. In some embodiments, cell surface removal of EGFR on the cancer cell is at least 20% or more relative to EGFR on the control cancer cell not contacted with the binding agent.

In some embodiments, cell surface removal of EGFR on the cancer cell is at least 20% or more relative to EGFR on the control cancer cell contacted with a monospecific EGFR binding agent. In some embodiments, internalization of EGFR in the cancer cell is at least 20% or more relative to internalizing of EGFR in the control cancer cell not contacted with the binding agent. In some embodiments, internalization of EGFR on the cancer cell is at least 20% or more relative to EGFR on the control cancer cell contacted with a monospecific EGFR binding agent. In some embodiments, degradation of EGFR in the cancer cell is at least 20% or more relative to degradation of EGFR in the control cancer cell not contacted with the binding agent. In some embodiments, degradation of EGFR on the cancer cell is at least 20% or more relative to EGFR on the control cancer cell contacted with a monospecific EGFR binding agent.

In some embodiments, the monospecific EGFR binding agent is Cetuximab. In some embodiments, the method increases the susceptibility of the cancer cell to cancer therapeutic agents. In some embodiments, the cancer therapeutic agent is a cytotoxic agent. In some embodiments, the method reduces proliferation of the cancer cell. In some embodiments, the method increases death of the cancer cell. In some embodiments, the contacting is performed in vivo.

In another aspect, the present disclosure provides a method for treating cancer in a subject, the method comprising: administering to a subject a binding agent, wherein the binding agent comprises: (i) a first binding domain that specifically binds to an endogenous internalizing receptor, wherein the endogenous internalizing receptor is expressed on a target cell, and wherein the endogenous internalizing receptor is B7-H3; (ii) a second binding domain that specifically binds to the target protein, wherein the target protein comprises EGFR.

In some embodiments, the cancer is breast cancer, B cell lymphoma, pancreatic cancer, Hodgkin's lymphoma, ovarian cancer, prostate cancer, mesothelioma, lung cancer, non-Hodgkin's B-cell (B-NHL) lymphoma, melanoma, chronic lymphocytic leukemia, acute lymphocytic leukemia, neuroblastoma, glioma, glioblastoma, bladder cancer, colorectal cancer, or head and neck cancer.

In some embodiments, tumor volume of the tumor contacted with the multispecific binding agent decreases by 20% or more relative to the tumor volume of a tumor not contacted with the bispecific binding agent. In some embodiments, tumor volume of the tumor contacted with the multispecific binding agent is less than 80% or less relative to the tumor volume of a tumor not contacted with the bispecific binding agent. In some embodiments, expression of EGFR on the cancer cell decreases by 20% relative to the EGFR expression of a cancer cell not contacted with the bispecific binding agent. In some embodiments, expression of EGFR on the cancer cell decreases by 20% relative to the EGFR expression of a cancer cell contacted with a monospecific EGFR binding agent. In some embodiments, the monospecific EGFR binding agent is Cetuximab.

In another aspect, the present disclosure provides a multispecific binding agent comprising: (a) a first binding domain that specifically binds to an endogenous internalizing receptor, wherein the endogenous internalizing receptor is B7-H3; and (b) a second binding domain that specifically binds to a target protein, wherein the target protein is EGFR.

In some embodiments, the multispecific binding agent is a multispecific antibody, bispecific antibody, a bispecific diabody, a bispecific Fab2, bispecific camelid antibody, a bispecific peptibody scFv-Fc, a bispecific IgG, a knob and hole bispecific IgG, a Fc-Fab, or a knob and hole bispecific Fc-Fab.

In some embodiments, the first binding domain binds to an epitope of the endogenous internalizing receptor on the target cell, wherein the epitope comprises at least 80% sequence identity to an epitope to which an antibody comprising SEQ ID NOs: 99 and 101 binds. In some embodiments, the first binding domain binds to an epitope of the endogenous internalizing receptor on the target cell, wherein the epitope comprises at least 90% sequence identity to the epitope to which the antibody comprising SEQ ID NOs: 99 and 101 binds. In some embodiments, the first binding domain binds to an epitope of the endogenous internalizing receptor on the target cell that does not include any of the amino acids from the epitope to which the antibody comprising SEQ ID NOs: 99 and 101 binds.

In some embodiments, the second binding domain binds to an epitope of the target protein on the target cell, wherein the epitope comprises at least 80% sequence identity to an epitope to which Cetuximab binds. In some embodiments, the second binding domain binds to an epitope of the target protein on the target cell, wherein the epitope comprises at least 90% sequence identity to the epitope to which Cetuximab binds. In some embodiments, the second binding domain binds to an epitope of the target protein on the target cell that does not include any of the amino acids from the epitope to which Cetuximab binds.

In some embodiments, the second binding domain binds to an epitope of the target protein on the target cell, wherein the epitope comprises at least 80% sequence identity to an epitope to which Mav2 binds. In some embodiments, the second binding domain binds to an epitope of the target protein on the target cell, wherein the epitope comprises at least 90% sequence identity to the epitope to which Mav2 binds. In some embodiments, the second binding domain binds to an epitope of the target protein on the target cell that does not include any of the amino acids from the epitope to which Mav2 binds.

In some embodiments, the first binding domain comprises a first binding domain variable heavy chain and a first binding domain variable light chain. In some embodiments, the first binding domain variable heavy chain comprises at least 80% sequence identity to SEQ ID NO: 99. In some embodiments, the first binding domain variable heavy chain comprises at least 90% sequence identity to SEQ ID NO: 99. In some embodiments, the first binding domain variable heavy chain comprises SEQ ID NO: 99. In some embodiments, the first binding domain variable light chain comprises at least 80% sequence identity to SEQ ID NO: 101. In some embodiments, the first binding domain variable light chain comprises at least 90% sequence identity to SEQ ID NO: 101. In some embodiments, the first binding domain variable light chain comprises SEQ ID NO: 101.

In some embodiments, the second binding domain comprises a second binding domain variable heavy chain. In some embodiments, the second binding domain variable heavy chain comprises at least 80% sequence identity to SEQ ID NO: 655. The multispecific binding agent of claim 216, wherein the second binding domain variable heavy chain comprises at least 90% sequence identity to SEQ ID NO: 655. In some embodiments, the second binding domain variable heavy chain comprises SEQ ID NO: 655.

In some embodiments, the half-life of the multispecific binding agent is within 20% of the half-life of Cetuximab. In some embodiments, the clearance rate of the multispecific binding agent is within 20-95% of the clearance rate of Cetuximab. In some embodiments, the Kd of the multispecific binding agent is within two-fold of the binding affinity of Cetuximab to EGFR. In some embodiments, the Kd of the multispecific binding agent is within five-fold of the binding affinity of Cetuximab to EGFR. In some embodiments, the Kd of the multispecific binding agent is within ten-fold of the binding affinity of Cetuximab to EGFR.

In some embodiments, the Kd of the binding affinity of the multispecific binding agent may be within an order of magnitude of the binding affinity of a monovalent binding agent. In some embodiments, the Kd of the multispecific binding agent is within +/−10% of the binding affinity of Cetuximab to EGFR. In some embodiments, the Kd of the multispecific binding agent is within +/−20% of the binding affinity of Cetuximab to EGFR. In some embodiments, the Kd of the multispecific binding agent is within +/−30% of the binding affinity of Cetuximab to EGFR. In some embodiments, the Kd of the multispecific binding agent is less than the binding affinity of Cetuximab to EGFR. In some embodiments, the Kd of the multispecific binding agent is more than the binding affinity of Cetuximab to EGFR.

In another aspect, the present disclosure provides a method of degrading a target protein on a surface of a target cell, the method comprising: contacting an E3 ligase and the target protein on the surface of the target cell with a binding agent, wherein the binding agent comprises: (i) a first binding domain that specifically binds to a E3 ligase, wherein the E3 ligase is RNF43; (ii) a second binding domain that specifically binds to the target protein, wherein the target protein is EGFR.

In some embodiments, the binding agent is a multispecific antibody, a bispecific diabody, a bispecific Fab2, bispecific camelid antibody, a bispecific peptibody scFv-Fc, a bispecific IgG, a knob and hole bispecific IgG, a Fc-Fab, or a knob and hole bispecific Fc-Fab.

In some embodiments, the first binding domain binds to an epitope of the endogenous internalizing receptor on the target cell, wherein the epitope comprises at least 80% sequence identity to an epitope to which an antibody comprising SEQ ID NOs: 331 and 333 binds. In some embodiments, the first binding domain binds to an epitope of the endogenous internalizing receptor on the target cell, wherein the epitope comprises at least 90% sequence identity to the epitope to which the antibody comprising SEQ ID NOs: 331 and 333 binds. In some embodiments, the first binding domain binds to an epitope of the endogenous internalizing receptor on the target cell that does not include any of the amino acids from the epitope to which the antibody comprising SEQ ID NOs: 331 and 333 binds.

In some embodiments, the second binding domain binds to an epitope of the target protein on the target cell, wherein the epitope comprises at least 80% sequence identity to an epitope to which Cetuximab binds. In some embodiments, the second binding domain binds to an epitope of the target protein on the target cell, wherein the epitope comprises at least 90% sequence identity to the epitope to which Cetuximab binds. In some embodiments, the second binding domain binds to an epitope of the target protein on the target cell that does not include any of the amino acids from the epitope to which Cetuximab binds.

In some embodiments, the second binding domain binds to an epitope of the target protein on the target cell, wherein the epitope comprises at least 80% sequence identity to an epitope to which Mav2 binds. In some embodiments, the second binding domain binds to an epitope of the target protein on the target cell, wherein the epitope comprises at least 90% sequence identity to the epitope to which Mav2 binds. In some embodiments, the second binding domain binds to an epitope of the target protein on the target cell that does not include any of the amino acids from the epitope to which Mav2 binds.

In some embodiments, the first binding domain comprises a first binding domain variable heavy chain and a first binding domain variable light chain. In some embodiments, the first binding domain variable heavy chain comprises at least 80%, sequence identity to SEQ ID NO: 331. In some embodiments, the first binding domain variable heavy chain comprises at least 90%, sequence identity to SEQ ID NO: 331. In some embodiments, the first binding domain variable heavy chain comprises SEQ ID NO: 331. In some embodiments, the first binding domain variable light chain comprises at least 80% sequence identity to SEQ ID NO: 333. In some embodiments, the first binding domain variable light chain comprises at least 90% sequence identity to SEQ ID NO: 333. In some embodiments, the first binding domain variable light chain comprises SEQ ID NO: 333.

In some embodiments, the second binding domain comprises a second binding domain variable heavy chain. In some embodiments, the second binding domain variable heavy chain comprises at least 80% sequence identity to SEQ ID NO: 655. In some embodiments, the second binding domain variable heavy chain comprises at least 90% sequence identity to SEQ ID NO: 655. In some embodiments, the second binding domain variable heavy chain comprises SEQ ID NO: 655.

In some embodiments, the E3 ligase is degraded. In some embodiments, the target cell is a cancer cell. In some embodiments, the cancer cell is selected from the group consisting of a breast cancer cell, a B cell lymphoma cell, a pancreatic cancer cell, a Hodgkin's lymphoma cell, an ovarian cancer cell, a prostate cancer cell, a mesothelioma cell, a lung cancer cell, a non-Hodgkin's B-cell (B-NHL) cell, a melanoma cell, a chronic lymphocytic leukemia cell, an acute lymphocytic leukemia cell, a neuroblastoma cell, a glioma cell, a glioblastoma cell, a bladder cancer cell, a colorectal cancer cell, and head and neck cancer cell.

In some embodiments, expression of EGFR on the cancer cell decreases following contact with the bispecific binding agent, as compared to a control cancer cell that is not contacted with the binding agent. In some embodiments, expression of EGFR on the cancer cell decreases by 50% or more relative to expression of EGFR on the control cancer cell not contacted with the binding agent. In some embodiments, expression of EGFR on the cancer cell decreases by 50% or more relative to expression of EGFR on the control cancer cell contacted with a monospecific EGFR binding agent. In some embodiments, cell surface removal of EGFR on the cancer cell is at least 20% or more relative to EGFR on the control cancer cell not contacted with the binding agent. In some embodiments, cell surface removal of EGFR on the cancer cell is at least 20% or more relative to EGFR on the control cancer cell contacted with a monospecific EGFR binding agent.

In some embodiments, internalization of EGFR in the cancer cell is at least 20% or more relative to internalizing of EGFR in the control cancer cell not contacted with the binding agent. In some embodiments, internalization of EGFR on the cancer cell is at least 20% or more relative to EGFR on the control cancer cell contacted with a monospecific EGFR binding agent. In some embodiments, degradation of EGFR in the cancer cell is at least 20% or more relative to degradation of EGFR in the control cancer cell not contacted with the binding agent. In some embodiments, cell degradation of EGFR on the cancer cell is at least 20% or more relative to EGFR on the control cancer cell contacted with a monospecific EGFR binding agent.

In some embodiments, the monospecific EGFR binding agent is Cetuximab. In some embodiments, the method increases the susceptibility of the cancer cell to cancer therapeutic agents. In some embodiments, the cancer therapeutic agent is a cytotoxic agent. In some embodiments, the method reduces proliferation of the cancer cell. In some embodiments, the method increases death of the cancer cell. In some embodiments, the contacting is performed in vivo.

In another aspect, the present disclosure provides a method for treating cancer in a subject, the method comprising: administering to a subject a binding agent, wherein the binding agent comprises: (i) a first binding domain that specifically binds to an E3 ligase, wherein the E3 ligase is RNF43; (ii) a second binding domain that specifically binds to the target protein, wherein the target protein comprises EGFR.

In some embodiments, the first binding domain comprises a first binding domain variable heavy chain and a first binding domain variable light chain. In some embodiments, the first binding domain variable heavy chain comprises at least 80%, sequence identity to SEQ ID NO: 331. In some embodiments, the first binding domain variable heavy chain comprises at least 90%, sequence identity to SEQ ID NO: 331. In some embodiments, the first binding domain variable heavy chain comprises SEQ ID NO: 331. In some embodiments, the first binding domain variable light chain comprises at least 80% sequence identity to SEQ ID NO: 333. In some embodiments, the first binding domain variable light chain comprises at least 90% sequence identity to SEQ ID NO: 333. In some embodiments, the first binding domain variable light chain comprises SEQ ID NO: 333.

In some embodiments, the second binding domain comprises a second binding domain variable heavy chain. In some embodiments, the second binding domain variable heavy chain comprises at least 80% sequence identity to SEQ ID NO: 655. In some embodiments, the second binding domain variable heavy chain comprises at least 90% sequence identity to SEQ ID NO: 655. In some embodiments, the second binding domain variable heavy chain comprises SEQ ID NO: 655.

In some embodiments, the cancer is breast cancer, B cell lymphoma, pancreatic cancer, Hodgkin's lymphoma, ovarian cancer, prostate cancer, mesothelioma, lung cancer, non-Hodgkin's B-cell (B-NHL) lymphoma, melanoma, chronic lymphocytic leukemia, acute lymphocytic leukemia, neuroblastoma, glioma, glioblastoma, bladder cancer, colorectal cancer, or head and neck cancer.

In another aspect, the present disclosure provides a multispecific binding agent comprising: (a) a first binding domain that specifically binds to an E3 ligase, wherein the E3 ligase is RNF43; and (b) a second binding domain that specifically binds to a target protein, wherein the target protein is EGFR.

In some embodiments, the multispecific binding agent is a multispecific antibody, bispecific antibody, a bispecific diabody, a bispecific Fab2, bispecific camelid antibody, a bispecific peptibody scFv-Fc, a bispecific IgG, a knob and hole bispecific IgG, a Fc-Fab, or a knob and hole bispecific Fc-Fab.

In some embodiments, the first binding domain binds to an epitope of RNF43 on the target cell, wherein the epitope comprises at least 80% sequence identity to an epitope to which an antibody comprising SEQ ID NOs: 331 and 333 binds. In some embodiments, the first binding domain binds to an epitope of RNF43 on the target cell, wherein the epitope comprises at least 90% sequence identity to the epitope to which the antibody comprising SEQ ID NOs: 331 and 333 binds. In some embodiments, the first binding domain binds to an epitope of RNF43 on the target cell that does not include any of the amino acids from the epitope to which the antibody comprising SEQ ID NOs: 331 and 333 binds.

In some embodiments, the second binding domain binds to an epitope of the target protein on the target cell, wherein the epitope comprises at least 80% sequence identity to an epitope to which Cetuximab binds. In some embodiments, the second binding domain binds to an epitope of the target protein on the target cell, wherein the epitope comprises at least 90% sequence identity to the epitope to which Cetuximab binds. In some embodiments, the second binding domain binds to an epitope of the target protein on the target cell that does not include any of the amino acids from the epitope to which Cetuximab binds.

In some embodiments, the second binding domain binds to an epitope of the target protein on the target cell, wherein the epitope comprises at least 80% sequence identity to an epitope to which Mav2 binds. In some embodiments, the second binding domain binds to an epitope of the target protein on the target cell, wherein the epitope comprises at least 90% sequence identity to the epitope to which Mav2 binds. In some embodiments, the second binding domain binds to an epitope of the target protein on the target cell that does not include any of the amino acids from the epitope to which Mav2 binds.

In some embodiments, the first binding domain comprises a first binding domain variable heavy chain and a first binding domain variable light chain. In some embodiments, the first binding domain variable heavy chain comprises at least 80% sequence identity to SEQ ID NO: 331. In some embodiments, the first binding domain variable heavy chain comprises at least 90% sequence identity to SEQ ID NO: 331. In some embodiments, the first binding domain variable heavy chain comprises SEQ ID NO: 331. In some embodiments, the first binding domain variable light chain comprises at least 80% sequence identity to SEQ ID NO: 333. In some embodiments, the first binding domain variable light chain comprises at least 90% sequence identity to SEQ ID NO: 333. In some embodiments, the first binding domain variable light chain comprises SEQ ID NO: 333.

In some embodiments, the second binding domain comprises a second binding domain variable heavy chain. In some embodiments, the second binding domain variable heavy chain comprises at least 80% sequence identity to SEQ ID NO: 655. In some embodiments, the second binding domain variable heavy chain comprises at least 90% sequence identity to SEQ ID NO: 655. In some embodiments, the second binding domain variable heavy chain comprises SEQ ID NO: 655.

In another aspect, the present disclosure provides a method of degrading a target protein on a surface of a target cell, the method comprising: contacting an endogenous internalizing receptor and the target protein on the surface of the target cell with a binding agent, wherein the binding agent comprises: (i) a first binding domain that specifically binds to an endogenous internalizing receptor, wherein the endogenous internalizing receptor is selected from the group consisting of LGR5, HER3, LY75, MST1R, MSLN, EpCAM, TNFRSF10B, and CD71; (ii) a second binding domain that specifically binds to the target protein, wherein the target protein comprises EGFR.

In some embodiments, the binding agent is a multispecific antibody, a bispecific diabody, a bispecific Fab2, bispecific camelid antibody, a bispecific peptibody scFv-Fc, a bispecific IgG, a knob and hole bispecific IgG, a Fc-Fab, or a knob and hole bispecific Fc-Fab.

In some embodiments, the first binding domain binds to an epitope of the endogenous internalizing receptor on the target cell, wherein the epitope comprises at least 80% sequence identity to an epitope to which an antibody comprising any one of a variable heavy chain sequence or any one of a variable light chain sequences listed in Table 1 or Table 2 binds. In some embodiments, the first binding domain binds to an epitope of the endogenous internalizing receptor on the target cell, wherein the epitope comprises at least 90% sequence identity to the epitope to which the antibody comprising any one of the variable heavy chain sequence or any one of the variable light chain sequences listed in Table 1 or Table 2 binds. In some embodiments, the first binding domain binds to an epitope of the endogenous internalizing receptor on the target cell that does not include any of the amino acids from the epitope to which the antibody comprising any one of the variable heavy chain sequence or any one of the variable light chain sequences listed in Table 1 or Table 2 binds.

In some embodiments, the second binding domain binds to an epitope of the target protein on the target cell, wherein the epitope comprises at least 80% sequence identity to an epitope to which Cetuximab binds. In some embodiments, the second binding domain binds to an epitope of the target protein on the target cell, wherein the epitope comprises at least 90% sequence identity to the epitope to which Cetuximab binds. In some embodiments, the second binding domain binds to an epitope of the target protein on the target cell that does not include any of the amino acids from the epitope to which Cetuximab binds.

In some embodiments, the second binding domain binds to an epitope of the target protein on the target cell, wherein the epitope comprises at least 80% sequence identity to an epitope to which Mav2 binds. In some embodiments, the second binding domain binds to an epitope of the target protein on the target cell, wherein the epitope comprises at least 90% sequence identity to the epitope to which Mav2 binds. In some embodiments, the second binding domain binds to an epitope of the target protein on the target cell that does not include any of the amino acids from the epitope to which Mav2 binds.

In some embodiments, the first binding domain comprises a first binding domain variable heavy chain and a first binding domain variable light chain. In some embodiments, the first binding domain variable heavy chain comprises at least 80%, sequence identity to any one of a variable heavy chain sequences listed in Table 1. In some embodiments, the first binding domain variable heavy chain comprises at least 90%, sequence identity to any one of the variable heavy chain sequences listed in Table 1. In some embodiments, the first binding domain variable heavy chain comprises any one of the variable heavy chain sequences listed in Table 1. In some embodiments, the first binding domain variable light chain comprises at least 80% sequence identity to any one of a variable light chain sequences listed in Table 1. In some embodiments, the first binding domain variable light chain comprises at least 90% sequence identity to any one of the variable light chain sequences listed in Table 1. In some embodiments, the first binding domain variable light chain comprises any one of the variable light chain sequences listed in Table 1.

In some embodiments, the second binding domain comprises a second binding domain variable heavy chain. In some embodiments, the second binding domain variable heavy chain comprises at least 80% sequence identity to SEQ ID NO: 655. In some embodiments, the second binding domain variable heavy chain comprises at least 90% sequence identity to SEQ ID NO: 655. In some embodiments, the second binding domain variable heavy chain comprises SEQ ID NO: 655.

In some embodiments, the endogenous internalizing receptor is recycled to the target cell surface following the internalization of the binding agent. In some embodiments, the endogenous internalizing receptor is degraded. In some embodiments, the target cell is a cancer cell. In some embodiments, the cancer cell is selected from the group consisting of a breast cancer cell, a B cell lymphoma cell, a pancreatic cancer cell, a Hodgkin's lymphoma cell, an ovarian cancer cell, a prostate cancer cell, a mesothelioma cell, a lung cancer cell, a non-Hodgkin's B-cell (B-NHL) cell, a melanoma cell, a chronic lymphocytic leukemia cell, an acute lymphocytic leukemia cell, a neuroblastoma cell, a glioma cell, a glioblastoma cell, a bladder cancer cell, a colorectal cancer cell, and head and neck cancer cell.

In some embodiments, expression of EGFR on the cancer cell decreases following contact with the multispecific binding agent, as compared to a control cancer cell that is not contacted with the binding agent. In some embodiments, expression of EGFR on the cancer cell decreases by 50% or more relative to expression of EGFR on the control cancer cell not contacted with the binding agent. In some embodiments, expression of EGFR on the cancer cell decreases by 50% or more relative to expression of EGFR on the control cancer cell contacted with a monospecific EGFR binding agent. In some embodiments, cell surface removal of EGFR on the cancer cell is at least 20% or more relative to EGFR on the control cancer cell not contacted with the binding agent. In some embodiments, cell surface removal of EGFR on the cancer cell is at least 20% or more relative to EGFR on the control cancer cell contacted with a monospecific EGFR binding agent.

In some embodiments, internalization of EGFR in the cancer cell is at least 20% or more relative to internalizing of EGFR in the control cancer cell not contacted with the binding agent. In some embodiments, internalization of EGFR on the cancer cell is at least 20% or more relative to EGFR on the control cancer cell contacted with a monospecific EGFR binding agent. In some embodiments, degradation of EGFR in the cancer cell is at least 20% or more relative to degradation of EGFR in the control cancer cell not contacted with the binding agent. In some embodiments, cell degradation of EGFR on the cancer cell is at least 20% or more relative to EGFR on the control cancer cell contacted with a monospecific EGFR binding agent.

In some embodiments, the monospecific EGFR binding agent is Cetuximab. In some embodiments, the method increases the susceptibility of the cancer cell to cancer therapeutic agents. In some embodiments, the cancer therapeutic agent is a cytotoxic agent. In some embodiments, the method reduces proliferation of the cancer cell. In some embodiments, the method increases death of the cancer cell. In some embodiments, the contacting is performed in vivo.

In another aspect, the present disclosure provides a method for treating cancer in a subject, the method comprising: administering to a subject a binding agent, wherein the binding agent comprises: (i) a first binding domain that specifically binds to an endogenous internalizing receptor, wherein the endogenous internalizing receptor is expressed on a target cell, and wherein the endogenous internalizing receptor is selected from the group consisting of LGR5, HER3, LY75, MST1R, MSLN, EpCAM, TNFRSF10B, and CD71; (ii) a second binding domain that specifically binds to the target protein, wherein the target protein comprises EGFR.

In some embodiments, the cancer is breast cancer, B cell lymphoma, pancreatic cancer, Hodgkin's lymphoma, ovarian cancer, prostate cancer, mesothelioma, lung cancer, non-Hodgkin's B-cell (B-NHL) lymphoma, melanoma, chronic lymphocytic leukemia, acute lymphocytic leukemia, neuroblastoma, glioma, glioblastoma, bladder cancer, colorectal cancer, or head and neck cancer. In some embodiments, tumor volume of the tumor contacted with the multispecific binding agent decreases by at least 20% or more relative to the tumor volume of a tumor not contacted with the bispecific binding agent. In some embodiments, tumor volume of the tumor contacted with the multispecific binding agent is less than 80% or less relative to the tumor volume of a tumor not contacted with the bispecific binding agent. In some embodiments, expression of EGFR on the cancer cell decreases by at least 20% relative to the EGFR expression of a cancer cell not contacted with the bispecific binding agent. In some embodiments, expression of EGFR on the cancer cell decreases by 20% relative to the EGFR expression of a cancer cell contacted with a monospecific EGFR binding agent. In some embodiments, the monospecific EGFR binding agent is Cetuximab.

In another aspect, the present disclosure provides a multispecific binding agent comprising: (a) a first binding domain that specifically binds to an endogenous internalizing receptor, wherein the endogenous internalizing receptor is selected from the group consisting of LGR5, HER3, LY75, MST1R, MSLN, EpCAM, TNFRSF10B, and CD71; and (b) a second binding domain that specifically binds to a target protein, wherein the target protein is EGFR.

In some embodiments, the multispecific binding agent is a multispecific antibody, bispecific antibody, a bispecific diabody, a bispecific Fab2, bispecific camelid antibody, a bispecific peptibody scFv-Fc, a bispecific IgG, a knob and hole bispecific IgG, a Fc-Fab, or a knob and hole bispecific Fc-Fab.

In some embodiments, the first binding domain binds to an epitope of the endogenous internalizing receptor on the target cell, wherein the epitope comprises at least 80% sequence identity to an epitope to which an antibody comprising any one of a variable heavy chain sequence or any one of a variable light chain sequences listed in Table 1 or Table 2 binds. In some embodiments, the first binding domain binds to an epitope of the endogenous internalizing receptor on the target cell, wherein the epitope comprises at least 90% sequence identity to the epitope to which the antibody comprising any one of the variable heavy chain sequence or any one of the variable light chain sequences listed in Table 1 or Table 2 binds. In some embodiments, the first binding domain binds to an epitope of the endogenous internalizing receptor on the target cell that does not include any of the amino acids from the epitope to which the antibody comprising any one of the variable heavy chain sequence or any one of the variable light chain sequences listed in Table 1 or Table 2 binds.

In some embodiments, the second binding domain binds to an epitope of the target protein on the target cell, wherein the epitope comprises at least 80% sequence identity to an epitope to which Cetuximab binds. In some embodiments, the second binding domain binds to an epitope of the target protein on the target cell, wherein the epitope comprises at least 90% sequence identity to the epitope to which Cetuximab binds. In some embodiments, the second binding domain binds to an epitope of the target protein on the target cell that does not include any of the amino acids from the epitope to which Cetuximab binds.

In some embodiments, the second binding domain binds to an epitope of the target protein on the target cell, wherein the epitope comprises at least 80% sequence identity to an epitope to which Mav2 binds. In some embodiments, the second binding domain binds to an epitope of the target protein on the target cell, wherein the epitope comprises at least 90% sequence identity to the epitope to which Mav2 binds. In some embodiments, the second binding domain binds to an epitope of the target protein on the target cell that does not include any of the amino acids from the epitope to which Mav2 binds.

In some embodiments, the first binding domain comprises a first binding domain variable heavy chain and a first binding domain variable light chain. In some embodiments, the first binding domain variable heavy chain comprises at least 80% sequence identity to any one of a variable heavy chain sequence listed in Table 1. In some embodiments, the first binding domain variable heavy chain comprises at least 90% sequence identity to any one of the variable heavy chain sequences listed in Table 1. In some embodiments, the first binding domain variable heavy chain comprises any one of the variable heavy chain sequences listed in Table 1. In some embodiments, the first binding domain variable light chain comprises at least 80% sequence identity to any one of a variable light chain sequences listed in Table 1. In some embodiments, the first binding domain variable light chain comprises at least 90% sequence identity to any one of the variable light chain sequences listed in Table 1. In some embodiments, the first binding domain variable light chain comprises any one of the variable light chain sequences listed in Table 1.

In some embodiments, the second binding domain comprises a second binding domain variable heavy chain. In some embodiments, the second binding domain variable heavy chain comprises at least 80% sequence identity to SEQ ID NO: 655. In some embodiments, the second binding domain variable heavy chain comprises at least 90% sequence identity to SEQ ID NO: 655. In some embodiments, the second binding domain variable heavy chain comprises SEQ ID NO: 655.

In some embodiments, the half-life of the multispecific binding agent is within 20% of the half-life of Cetuximab. In some embodiments, the clearance rate of the multispecific binding agent is within 20-95% of the clearance rate of Cetuximab. In some embodiments, the Kd of the multispecific binding agent is within two-fold of the binding affinity of Cetuximab to EGFR. In some embodiments, the Kd of the multispecific binding agent is within five-fold of the binding affinity of Cetuximab to EGFR. In some embodiments, the Kd of the multispecific binding agent is within ten-fold of the binding affinity of Cetuximab to EGFR.

In some embodiments, the Kd of the binding affinity of the multispecific binding agent may be within an order of magnitude of the binding affinity of a monovalent binding agent. In some embodiments, the Kd of the multispecific binding agent is within +/−10% of the binding affinity of Cetuximab to EGFR. In some embodiments, the Kd of the multispecific binding agent is within +/−20% of the binding affinity of Cetuximab to EGFR. In some embodiments, the Kd of the multispecific binding agent is within +/−30% of the binding affinity of Cetuximab to EGFR. In some embodiments, the Kd of the multispecific binding agent is less than the binding affinity of Cetuximab to EGFR. In some embodiments, the Kd of the multispecific binding agent is more than the binding affinity of Cetuximab to EGFR.

In one aspect, the present disclosure provides a method of degrading an EGFR protein on a target cell, the method comprising: contacting the EGFR protein and a membrane-associated internalizing protein on the target cell with a bispecific binding agent, wherein the contacting of the EGFR protein and the membrane-associated internalizing protein with the bispecific binding agent leads to internalization and degradation of the EGFR protein; and wherein the bispecific binding agent comprises: (a) a first binding domain that specifically binds to an extracellular epitope the membrane associated internalizing protein; and (b) a second binding domain that specifically binds to an extracellular epitope on the EGFR protein; wherein the membrane associated internalizing protein is selected from CEACAM5, CEACAM6, HER3, MUC1, CD205, CD166, PRLR, SLC34A2, ITGB6, LRRC15, MUC16, SLC39A6, AXL, CD40, CD228, MUC5A, ITGB1, STn, KAAG1, DLKT, 5T4, SEZ6, ADAM9, I-Ag7, ENPP3, CD46, CD56, ROR1, GPR20, TM4SF1, B7-H4, ALPP, LY6E, CLDN18, LY6G6D, GPR56, CD71, LGR5, LY75, CD276/B7-H3, MST1R, MSLN, EpCAM, TNFRSF10B, STEAP1, MELTF, TROP2, CDH17, RNF43, and RNF128.

In some embodiments, the membrane associated internalizing protein is selected from CD205, CD166, SLC34A2, ITGB6, LRRC15, and MUC16. In some embodiments, the bispecific binding agent comprises an antibody. In some embodiments, the bispecific binding agent comprises a bispecific antibody.

In one aspect, the present disclosure provides a bispecific binding agent comprising a bispecific antibody or antibody derivative, the bispecific binding agent comprising: (a) a first binding domain that specifically binds to an extracellular epitope of an EGFR protein of a target cell; and (b) a second binding domain that specifically binds to an extracellular epitope of a membrane-associated internalizing protein on a target cell; wherein the membrane associated internalizing protein is selected from CD205, CD166, SLC34A2, ITGB6, LRRC15, MUC16, SLC39A6, AXL, CD40, CD228, MUC5A, ITGB1, STn, KAAG1, DLK1, 5T4, SEZ6, ADAM9, I-Ag7, ENPP3, CD46, CD56, ROR1, GPR20, TM4SF1, B7-H4, ALPP, LY6E, CLDN18, LY6G6D, GPR56, CD71, CECAM5, LGR5, LY75, CD276/B7-H3, MST1R, MSLN, EpCAM, TNFRSF10B, STEAP1, MELTF, TROP2, CDH17, RNF43, and RNF128.

In some embodiments, the membrane associated internalizing protein is CEACAM5. In some embodiments, the membrane associated internalizing protein is CEACAM6. In some embodiments, the membrane associated internalizing protein is HER3. In some embodiments, the membrane associated internalizing protein is MUC1. In some embodiments, the membrane associated internalizing protein is CD205. In some embodiments, the membrane associated internalizing protein is CD166. In some embodiments, the membrane associated internalizing protein is PRLR. In some embodiments, the membrane associated internalizing protein is SLC34A2. In some embodiments, the membrane associated internalizing protein is ITGB6. In some embodiments, the membrane associated internalizing protein is LRRC15. In some embodiments, the membrane associated internalizing protein is MUC16.

In some embodiments, the membrane associated internalizing protein is SLC39A6. In some embodiments, the membrane associated internalizing protein is AXL. In some embodiments, the membrane associated internalizing protein is MMP14. In some embodiments, the membrane associated internalizing protein is CMET. In some embodiments, the membrane associated internalizing protein is CD40. In some embodiments, the membrane associated internalizing protein is CD228A. In some embodiments, the membrane associated internalizing protein is CD70. In some embodiments, the membrane associated internalizing protein is MUC5A. In some embodiments, the membrane associated internalizing protein is CD44. In some embodiments, the membrane associated internalizing protein is ITGB1. In some embodiments, the membrane associated internalizing protein is STn. In some embodiments, the membrane associated internalizing protein is KAAG1. In some embodiments, the membrane associated internalizing protein is DLK1. In some embodiments, the membrane associated internalizing protein is 5T4. In some embodiments, the membrane associated internalizing protein is SEZ6. In some embodiments, the membrane associated internalizing protein is CD123. In some embodiments, the membrane associated internalizing protein is ADAM9. In some embodiments, the membrane associated internalizing protein is I-Ag7. In some embodiments, the membrane associated internalizing protein is ENPP3. In some embodiments, the membrane associated internalizing protein is CD37. In some embodiments, the membrane associated internalizing protein is CD46. In some embodiments, the membrane associated internalizing protein is CD56. In some embodiments, the membrane associated internalizing protein is CD74. In some embodiments, the membrane associated internalizing protein is IGF1R. In some embodiments, the membrane associated internalizing protein is ROR1. In some embodiments, the membrane associated internalizing protein is CDH6. In some embodiments, the membrane associated internalizing protein is ROR2. In some embodiments, the membrane associated internalizing protein is GPR20. In some embodiments, the membrane associated internalizing protein is TM4SF1. In some embodiments, the membrane associated internalizing protein is B7-H4. In some embodiments, the membrane associated internalizing protein is ALPP. In some embodiments, the membrane associated internalizing protein is LY6E. In some embodiments, the membrane associated internalizing protein is CLDN18. In some embodiments, the membrane associated internalizing protein is LY6G6D. In some embodiments, the membrane associated internalizing protein is GPR56. In some embodiments, the membrane associated internalizing protein is CD71.

In some embodiments, the membrane associated internalizing protein is LGR5. In some embodiments, the membrane associated internalizing protein is LY75. In some embodiments, the membrane associated internalizing protein is CD276/B7-H3. In some embodiments, the membrane associated internalizing protein is MST1R. In some embodiments, the membrane associated internalizing protein is MSLN. In some embodiments, the membrane associated internalizing protein is EpCAM. In some embodiments, the membrane associated internalizing protein is TNFRSF10B. In some embodiments, the membrane associated internalizing protein is STEAP1. In some embodiments, the membrane associated internalizing protein is MELTF. In some embodiments, the membrane associated internalizing protein is TROP2. In some embodiments, the membrane associated internalizing protein is CDH17. In some embodiments, the membrane associated internalizing protein is RNF43. In some embodiments, the membrane associated internalizing protein is RNF43.

In some embodiments, the bispecific binding agent comprises a knob and hole bispecific IgG. In some embodiments, the bispecific binding agent does not comprise an antibody-drug conjugate.

In another aspect, the present disclosure provides a pharmaceutical composition comprising a bispecific binding agent of agent of the present disclosure and a pharmaceutically acceptable excipient.

In another aspect, the present disclosure provides a method of treating cancer in a subject in need thereof, the method comprising administering to the subject a bispecific binding agent of the present disclosure or a pharmaceutical composition comprising a bispecific binding agent of agent of the present disclosure and a pharmaceutically acceptable excipient.

In another aspect, the present disclosure provides a method of arresting growth of a target cell, the method comprising contacting the cell with a bispecific binding agent of the present disclosure. In some embodiments, the cell is a cancer cell.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the present disclosure are set forth with particularity in the appended claims. A better understanding of the features and advantages of the present disclosure will be obtained by reference to the following detailed description that sets forth illustrative embodiments, in which the principles of the present disclosure are utilized, and the accompanying drawings (also “figure” and “FIG.” herein), of which:

FIG. 1 depicts a method of the present disclosure in which degradation of a target protein 112 (i.e., EGFR) is mediated by binding of bifunctional binding agent 101.

FIGS. 2A-2D are charts depicting percentages of EGFR cell surface removal in multiple cell types when treated with different concentrations of various bispecific antibodies where the affinity of the degrading monoclonal antibody (Ab) varied in affinity from 1-1800 nM. FIG. 2A is a chart depicting the percentage of EGFR cell surface removal in NCI-H1975 cells treated with bispecific antibodies at 50 nM. FIG. 2B is a chart depicting the percentage of EGFR cell surface removal in NCI-H1975 cells treated with bispecific antibodies at 500 nM. FIG. 2C is a chart depicting the percentage of EGFR cell surface removal in HT29 cells treated with bispecific antibodies at 50 nM. FIG. 2D is a chart depicting the percentage of EGFR cell surface removal in HT29 cells treated with bispecific antibodies at 500 nM.

FIGS. 3A-3B are charts depicting percentage of EGFR cell surface removal on target cells treated with various bispecific antibodies. FIG. 3A is a chart depicting percentage of EGFR cell surface removal on NCIH1975 target cells treated with various bispecific antibodies at 500 nM concentration. FIG. 3B is a chart depicting percentage of EGFR cell surface removal on HT29 target cells treated with various bispecific antibodies at 500 nM concentration.

FIGS. 4A-4B are charts depicting cell surface removal of EGFR. FIG. 4A is a chart depicting cell surface removal of EGFR on target cells when treated with various bispecific antibodies where the antibody to the EGFR target binds to different epitopes. FIG. 4B is a chart depicting cell surface removal of EGFR on target cells when treated with various bispecific antibodies where the antibody to the degrader binds to different epitopes.

FIG. 5 is a chart depicting internalization of EGFR on target cells when treated with various bispecific antibodies where the bispecific drove internalization above either single arm mAb targeting either the target or degrader.

FIGS. 6A-6B are charts depicting internalization and degradation of EGFR on target cells when treated with various bispecific antibodies. FIG. 6A is a chart depicting internalization of EGFR on target cells when treated with various bispecific antibodies. FIG. 6B is a chart depicting whole cell degradation of EGFR on target cells when treated with various bispecific antibodies.

FIGS. 7A-7B depict the amount of EGFR in target cells treated with various bispecific antibodies. FIG. 7A is an image of a Western blot depicting amount of EGFR protein on target cells when treated with various bispecific antibodies. FIG. 7B is a chart depicting whole cell degradation of EGFR on target cells when treated with various bispecific antibodies.

FIGS. 8A-8B depict the amount of EGFR degraded in target cells treated with various bispecific antibodies. FIG. 8A is an image of a Western blot depicting amount of total EGFR protein on target cells when treated with various bispecific antibodies at different concentrations. FIG. 8B is a chart depicting percentage of degradation of EGFR on target cells when treated with various bispecific antibodies at different concentrations.

FIGS. 9A-9C depict degradation of EGFR in target cells treated with various bispecific antibodies. FIG. 9A is a chart depicting flow cytometry binding analysis by fluorescence in target cells treated with various bispecific antibodies. FIG. 9B is a schematic depicting an exemplary mechanism of EGFR degradation. FIG. 9C is an image of immunofluorescence staining in cancer spheroids.

FIGS. 10A-10G depict expression of EGFR in target cells treated with various bispecific antibodies. FIG. 10A is an image of a Western blot depicting amount of EGFR protein and phosphorylated EGFR protein on target cells when treated with various bispecific antibodies. FIG. 10B is an image of a Western blot depicting amount of EGFR protein and phosphorylated EGFR protein on target cells when treated with various bispecific antibodies.

FIG. 10C is an image of a Western blot depicting amount of EGFR protein and phosphorylated EGFR protein on target cells when treated with various bispecific antibodies at various concentrations. FIG. 10D is an image of tumor spheroids. FIG. 10E is a chart depicting quantification of tumor spheroids in samples treated with various bispecific antibodies. FIG. 10F is a chart depicting quantification of tumor spheroids in samples treated with various bispecific antibodies. FIG. 10G is a chart depicting quantification of tumor spheroids in samples treated with various bispecific antibodies.

FIGS. 11A-11H depict cancer endpoints in animals treated with bispecific antibodies. FIG. 11A is schematic depicting an exemplary workflow and treatment regimen.

FIG. 11B is a chart depicting tumor volume over time in animals treated with a bispecific antibody. FIG. 11C is a chart depicting tumor volume in animals treated with a bispecific antibody at different concentrations. FIG. 11D is a chart depicting tumor volume over time in animals treated with a bispecific antibody. FIG. 11E is an image depicting EGFR expression in cells treated with various bispecific antibodies. FIG. 11F is a chart depicting quantification of EGFR in cells treated with various bispecific antibodies. FIG. 11G is an image depicting p-EGFR expression in cells treated with various bispecific antibodies. FIG. 11H is a chart depicting quantification of p-EGFR in cells treated with various bispecific antibodies.

FIGS. 12A-12C depict pharmacokinetic endpoints in animals treated with bispecific antibodies. FIG. 12A is schematic depicting an exemplary workflow and treatment regimen. FIG. 12B is a chart depicting serum concentration of various bispecific antibodies over time in mice. FIG. 12C is a chart depicting serum concentration of various bispecific antibodies over time in mice.

### DETAILED DESCRIPTION

The present disclosure generally relates to multispecific binding agents, which bind to both a target protein, and a membrane-associated internalizing protein or a membrane-associated degrading protein present on the surface of a target cell. In some embodiments, the present disclosure provides methods of degrading a target protein comprising contacting the target protein with a binding agent that simultaneously binds and a membrane-associated internalizing protein, leading to cellular internalization of the target protein and subsequent degradation of the target protein. In other embodiments, the present disclosure provides methods of degrading a target protein comprising contacting the target protein with a binding agent that simultaneously binds a membrane-associated degrading protein, leading to degradation of the target protein.

### Definitions

Unless defined otherwise, all technical and scientific terms used herein have the same meaning as is commonly understood by one of skill in the art to which this disclosure belongs. All patents and publications referred to herein are incorporated by reference.

As used in the specification and claims, the singular form “a”, “an” and “the” includes plural references unless the context clearly dictates otherwise.

The terms “administer”, “administered”, “administers” and “administering” are defined as providing a composition to a subject via a route known in the art, including but not limited to intravenous, intraarterial, intrathecal, oral, parenteral, perineural, buccal, topical, transdermal, rectal, intramuscular, subcutaneous, intraosseous, transmucosal, intraperitoneal, or nerve root sheath routes of administration. In certain embodiments, oral routes of administering a composition can be used. The terms “administer”, “administered”, “administers” and “administering” a therapeutic protein should be understood to mean providing a therapeutic protein of the present disclosure or a prodrug of a therapeutic protein of the present disclosure to the individual in need.

The term “humanize” refers to replacement or substitution of certain amino acids in an antibody or nanobody derived from a non-human species, in particular in the framework regions and constant domains of the heavy and/or light chains, in order to avoid or minimize an immune response in humans.

As used herein, the terms “complementarity-determining region” or “CDR” within the context of antibodies or nanobodies refer to variable regions of either H (heavy) or L (light) chains (also abbreviated as VH and VL, respectively) and contains the amino acid sequences capable of specifically binding to antigenic targets. These CDR regions account for the basic specificity of the antibody for a particular antigenic determinant structure. Such regions are also referred to as “hypervariable regions.” The CDRs represent non-contiguous stretches of amino acids within the variable regions but, regardless of species, the positional locations of these critical amino acid sequences within the variable heavy and light chain regions have been found to have similar locations within the amino acid sequences of the variable chains. The variable heavy and light chains of all canonical antibodies each have three CDR regions, each non-contiguous with the others (termed L1, L2, L3, H1, H2, H3) for the respective light (L) and heavy (H) chains. Nanobodies, in particular, generally comprise a single amino acid chain that can be considered to comprise four “framework sequences or regions” or FRs and three complementarity-determining regions” or CDRs. The nanobodies have three CDR regions, each non-contiguous with the others (termed CDR1, CDR2, CDR3). The delineation of the FR and CDR sequences is based on the IMGT unique numbering system for V-domains and V-like domains.

As used herein, the terms “nucleic acid molecule,” “polynucleotide,” “polynucleic acid,” and “nucleic acid” are used interchangeably and refer to a polymeric form of nucleotides of any length, either deoxyribonucleotides or ribonucleotides, or analogs thereof. Polynucleotides may have any three-dimensional structure, and may perform any function, known or unknown. Non-limiting examples of polynucleotides include a gene, a gene fragment, exons, introns, messenger RNA (mRNA), transfer RNA, ribosomal RNA, ribozymes, cDNA, recombinant polynucleotides, branched polynucleotides, plasmids, vectors, isolated DNA of any sequence, control regions, isolated RNA of any sequence, nucleic acid probes, and primers. The nucleic acid molecule may be linear or circular.

A “nanobody” (Nb), as used herein, refers to the smallest antigen binding fragment or single variable domain (“VHH”) derived from naturally occurring heavy chain antibody and is known to the person skilled in the art. They are derived from heavy chain only antibodies, seen, for example, in camelid antibodies. The nanobodies hereof generally comprise a single amino acid chain that can be considered to comprise four “framework sequences” that make up the “scaffold” and three “complementarity-determining regions” or CDRs (as defined hereinbefore). It should be noted that the term “nanobody,” as used herein in its broadest sense, is not limited to a specific biological source or to a specific method of preparation.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable excipient” or “pharmaceutically acceptable carrier” as used herein means a pharmaceutically acceptable material, composition or vehicle, such as a liquid or solid filler, diluent, excipient, solvent or encapsulating material. Each carrier must be “acceptable” in the sense of being compatible with the other ingredients of the formulation and not injurious to the patient.

As used herein, the terms “polypeptide,” “protein,” and “peptide” are used interchangeably herein, and refer to a polymeric form of amino acids of any length, which can include coded and non-coded amino acids, chemically or biochemically modified or derivatized amino acids, and polypeptides having modified peptide backbones.

The terms “subject,” “individual,” and “patient” may be used interchangeably and refer to humans, as well as non-human mammals (e.g., non-human primates, canines, equines, felines, porcines, bovines, ungulates, lagomorphs, rodents, and the like). In various embodiments, the subject can be a human (e.g., adult male, adult female, adolescent male, adolescent female, male child, female child) under the care of a physician or other health worker in a hospital, as an outpatient, or other clinical context. In certain embodiments, the subject may not be under the care or prescription of a physician or other health worker.

As used herein, the phrase “a subject in need thereof” refers to a subject, as described infra, that suffers from, or is at risk for, a pathology to be prophylactically or therapeutically treated with a therapeutic protein described herein.

The term “specificity,” as used herein, refers to the ability of a protein binding domain, in particular, an immunoglobulin or an immunoglobulin fragment, such as a nanobody, to bind preferentially to one antigen versus a different antigen, and does not necessarily imply high affinity.

As used herein, “treatment” or “treating” refers to an approach for obtaining beneficial or desired results with respect to a disease, disorder, or medical condition including, but not limited to, a therapeutic benefit and/or a prophylactic benefit. In certain embodiments, treatment or treating involves administering a therapeutic protein or composition disclosed herein to a subject. A therapeutic benefit may include the eradication or amelioration of the underlying disorder being treated. Also, a therapeutic benefit may be achieved with the eradication or amelioration of one or more of the physiological symptoms associated with the underlying disorder, such as observing an improvement in the subject, notwithstanding that the subject may still be afflicted with the underlying disorder. In certain embodiments, for prophylactic benefit, the compositions are administered to a subject at risk of developing a particular disease, or to a subject reporting one or more of the physiological symptoms of a disease, even though a diagnosis of this disease may not have been made. Treating can include, for example, reducing, delaying or alleviating the severity of one or more symptoms of the disease or condition, or it can include reducing the frequency with which symptoms of a disease, defect, disorder, or adverse condition, and the like, are experienced by a patient. Treating can be used herein to refer to a method that results in some level of treatment or amelioration of the disease or condition, and can contemplate a range of results directed to that end, including but not restricted to prevention of the condition entirely.

In certain embodiments, the term “prevent” or “preventing” as related to a disease or disorder may refer to a compound that, in a statistical sample, reduces the occurrence of the disorder or condition in the treated sample relative to an untreated control sample, or delays the onset or reduces the severity of one or more symptoms of the disorder or condition relative to the untreated control sample.

A “therapeutic effect,” as that term is used herein, encompasses a therapeutic benefit and/or a prophylactic benefit as described above. A prophylactic effect includes delaying or eliminating the appearance of a disease or condition, delaying or eliminating the onset of symptoms of a disease or condition, slowing, halting, or reversing the progression of a disease or condition, or any combination thereof.

A “degrading protein” or “degrader protein,” as that term is used herein, may encompasses a range of moieties including, but not limited to membrane associated internalizing protein, an internalizing receptor, a membrane associated degrading receptor, a degrading receptor, a surface moiety configured to internalize a binding agent, a surface moiety configured to degrade a binding agent, combinations thereof, or variants thereof.

An “internalizing protein,” as that term is used here, may encompass a range of moieties including, but not limited to membrane associated internalizing protein, an internalizing receptor, a surface moiety configured to internalize a binding agent, combinations thereof, or variants thereof.

### Methods of Degrading EGFR Proteins

Epidermal Growth Factor Receptor (EGFR) is a transmembrane protein that is a receptor for extracellular protein ligands of the epidermal growth factor family (EGF family). EGFR is activated by binding of these specific ligands, including epidermal growth factor (EGF) and transforming growth factor α (TGFα). Aberrant EGFR function and/or expression is implicated in cancer, where it causes enhanced cell growth and division and drives tumor growth and invasion.

Mutations that lead to EGFR overexpression (known as upregulation or amplification) have been associated with a number of cancers, including adenocarcinoma of the lung cancer, anal cancers, glioblastoma and epithelian tumors of the head and neck. Mutations, amplifications or misregulations of EGFR or family members are implicated in about 30% of all epithelial cancers. Many of these somatic mutations involving EGFR lead to its constant activation, which produces uncontrolled cell division. Therefore, the degradation of EGFR in cancer is a promising treatment modality for cancer.

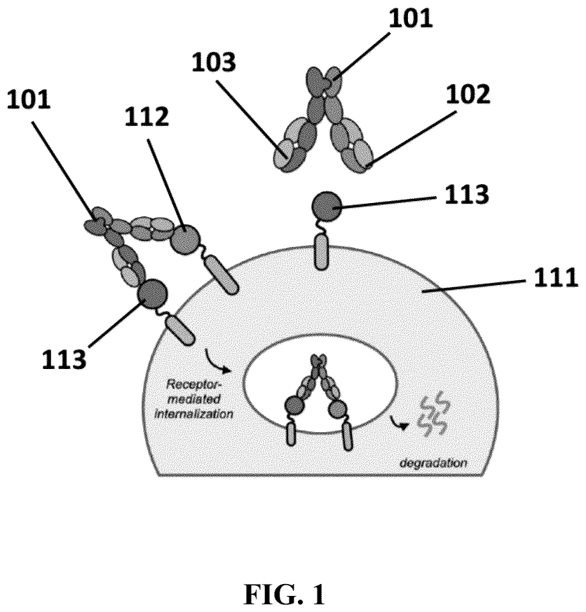

The present disclosure provides methods of degrading an EGFR protein on a target cell as shown in FIG. 1. The method utilizes a multispecific binding agent 101 that binds specifically to both (1) an extracellular epitope on the EGFR protein 112; and (2) an extracellular epitope on a membrane-associated internalizing protein 113 on a target cell 111. Bispecific binding agent 101 comprises first binding domain 102 that selectively binds to the EGFR protein 112 and second binding domain 103 that selectively binds to membrane-associated internalizing protein 113. Simultaneous binding of the multispecific binding agent 101 to the EGFR protein 112 and the membrane-associated internalizing protein 113 leads to internalization of both the EGFR protein 112 and the membrane-associated internalizing protein 113 into the target cell 111. Following internalization, the EGFR protein 112 is degraded by the target cell 111 (e.g., via trafficking to the lysosome).

In some embodiments, the membrane-associated internalizing protein is a cell-surface protein that internalizes upon binding of a binding agent (e.g., an antibody) to the protein. In some embodiments, the membrane-associated internalizing protein is selected from CEACAM5, CEACAM6, HER3, MUC1, CD205, CD166, PRLR, SLC34A2, ITGB6, LRRC15, MUC16, SLC39A6, AXL, CD40, CD228, MUC5A, ITGB1, STn, KAAG1, DLK1, 5T4, SEZ6, ADAM9, I-Ag7, ENPP3, CD46, CD56, ROR1, GPR20, TM4SF1, B7-H4, ALPP, LY6E, CLDN18, LY6G6D, GPR56, CD71, LGR5, LY75, CD276/B7-H3, MST1R, MSLN, EpCAM, TNFRSF10B, STEAP1, MELTF, TROP2, CDH17, RNF43, and RNF128.

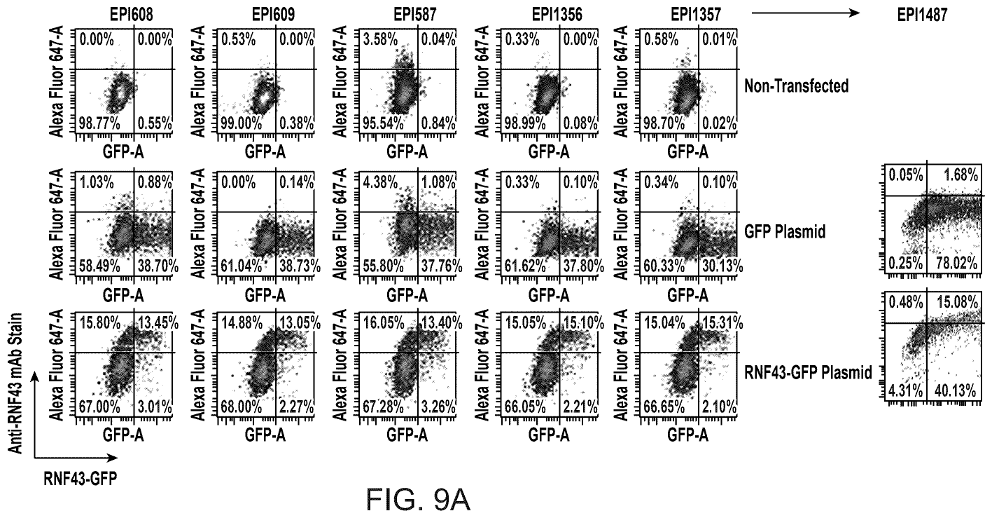

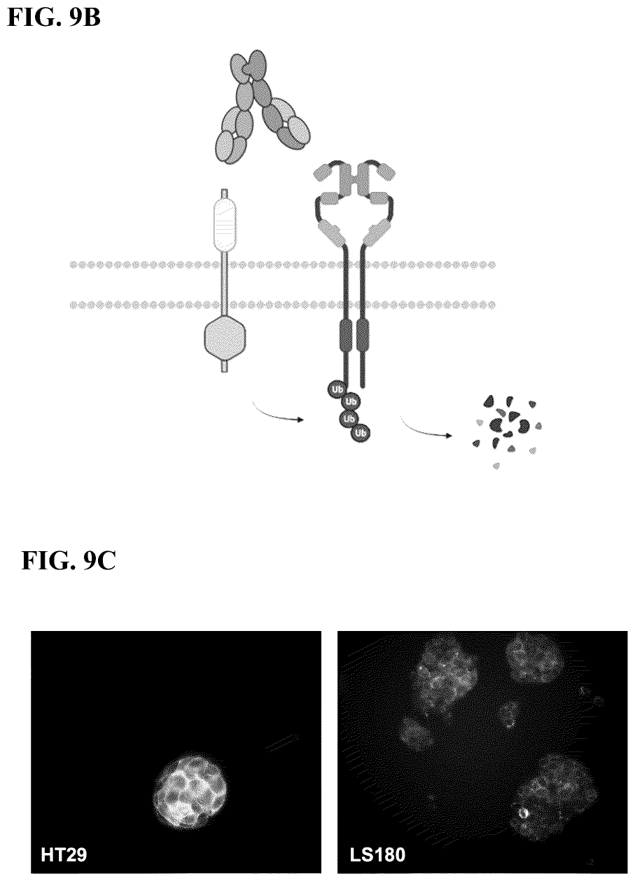

The present disclosure also provides methods of degrading an EGFR protein on a target cell as shown in FIG. 9B. The method utilizes a multispecific binding agent that binds specifically to both (1) an extracellular epitope on the EGFR protein; and (2) an extracellular epitope on a membrane-associated degrading protein on a target cell. Multispecific binding agent comprises first binding domain that selectively binds to the EGFR protein and second binding domain that selectively binds to membrane-associated degrading protein. Simultaneous binding of the multispecific binding agent to the EGFR protein and the membrane-associated degrading protein leads to degradation of both the EGFR protein and the membrane-associated degrading protein.

In some embodiments, the membrane-associated degrading protein is a cell-surface protein that degrades upon binding of a binding agent (e.g., an antibody) to the protein. In some embodiments, the membrane-associated degrading protein is RNF43.

In one aspect, the present disclosure provides a method of degrading an EGFR protein on a target cell, the method comprising:
contacting the EGFR protein and a membrane-associated internalizing protein on the target cell with a bispecific binding agent, wherein the contacting of the EGFR protein and the membrane-associated internalizing protein with the bispecific binding agent leads to internalization and degradation of the EGFR protein; and
wherein the bispecific binding agent comprises: (a) a first binding domain that specifically binds to an extracellular epitope the membrane associated internalizing protein; and (b) a second binding domain that specifically binds to an extracellular epitope on the EGFR protein;
wherein the membrane-associated internalizing protein is selected from CEACAM5, CEACAM6, HER3, MUC1, CD205, CD166, PRLR, SLC34A2, ITGB6, LRRC15, MUC16, SLC39A6, AXL, CD40, CD228, MUC5A, ITGB1, STn, KAAG1, DLK1, 5T4, SEZ6, ADAM9, I-Ag7, ENPP3, CD46, CD56, ROR1, GPR20, TM4SF1, B7-H4, ALPP, LY6E, CLDN18, LY6G6D, GPR56, CD71, LGR5, LY75, CD276/B7-H3, MST1R, MSLN, EpCAM, TNFRSF10B, STEAP1, MELTF, TROP2, CDH17, RNF43, and RNF128.

In some embodiments, the multispecific binding agent comprises an antibody. In some embodiments, the multispecific binding agent comprises a multispecific antibody. In some embodiments, the multispecific binding agent comprises a bispecific antibody. In some embodiments, the multispecific binding agent comprises an IgG antibody. In some embodiments, the multispecific binding agent comprises a multispecific IgG antibody. In some embodiments, the multispecific binding agent comprises a knob and hole bispecific IgG. In some embodiments, the multispecific binding agent is not an antibody-drug conjugate (“ADC”). In some embodiments, the multispecific binding agent comprises a bispecific binding agent. In some embodiments, the multispecific binding agent comprises a bispecific antibody. In some embodiments, the multispecific binding agent comprises a bispecific diabody. In some embodiments, the multispecific binding agent comprises a bispecific Fab2. In some embodiments, the multispecific binding agent comprises a bispecific camelid antibody. In some embodiments, the multispecific binding agent comprises a bispecific peptibody scFv-Fc. In some embodiments, the multispecific binding agent comprises Fc-Fab. In some embodiments, the multispecific binding agent comprises a knob and hole bispecific Fc-Fab.

### Multispecific Binding Agents

The multispecific binding agents of the present disclosure comprise at least two binding domains: one specific for a membrane-associated internalizing protein or a membrane-associated degrading protein, and the other specific for an EGFR protein. Multispecific binding agents of the disclosure include, without limitation, agents wherein the membrane-associated internalizing or degrading protein binding domain and the EGFR binding domain are each independently selected from an antibody (or half of an antibody), a nanobody, or a minibody, a Fab fragment, a single chain variable fragment (scFv), and a single domain antibody (sdAb), or a functional fragment thereof. These two binding domains can be the same type of molecule, or different. For example, multispecific binding agents of the disclosure include, without limitation, multispecific binding agents having an IgG that binds a membrane-associated internalizing or degrading protein, and an scFv domain that binds EGFR. The binding domains of the multispecific binding agent can be connected through covalent bonds, non-covalent interactions, or a combination thereof.

The multispecific binding agent can generally take the form of a protein, glycoprotein, lipoprotein, phosphoprotein, and the like. Some multispecific binding agent of the disclosure take the form of multispecific antibodies, bispecific antibodies or antibody derivatives. In some embodiments, the target protein binding domain is selected from the group consisting of a half antibody, a nanobody, or a minibody, a F(ab′)2 fragment, a Fab fragment, a single chain variable fragment (scFv), and a single domain antibody (sdAb), or a functional fragment thereof. The binding domains may together take the form of a bispecific antibody, a bispecific diabody, a bispecific camelid antibody or a bispecific peptibody, and the like. Antibody derivatives need not be derived from a specific wild type antibody. For example, one can employ known techniques such as phage display to generate and select for small proteins having a binding domain similar to an antibody complementarity-determining region (CDR). In some embodiments, the antigen-binding moiety includes an scFv. The binding domain can also be derived from a natural or synthetic ligand or receptor, whether soluble or membrane-bound, that specifically binds to the EGFR protein.

Multispecific antibodies can be prepared by known methods. Embodiments of the disclosure include “knob-into-hole” bispecific antibodies, wherein the otherwise symmetric dimerization region of a bispecific binding agent is altered so that it is asymmetric. For example, a knob-into-hole bispecific IgG that is specific for antigens A and B can be altered so that the Fc portion of the A-binding chain has one or more protrusions (“knobs”), and the Fc portion of the B-binding chain has one or more hollows (“holes”), where the knobs and holes are arranged to interact. This reduces the homodimerization (A-A and B-B antibodies), and promotes the heterodimerization desired for a bispecific binding agent. See, e.g., Y. Xu et al., mAbs (2015) 7(1):231-42. In some embodiments, the bispecific binding agent has a knob-into-hole design. In some embodiments, the “knob” comprises a T336W alteration of the CH3 domain, i.e., the threonine at position 336 is replaced by a tryptophan. In some embodiments, the “hole” comprises one or a combination of T366S, L368A, and Y407V. In some embodiments, the “hole” comprises T366S, L368A, and Y407V.

In some embodiments, the multispecific binding agent comprises an FcRn receptor recognition domain, to promote return of the bispecific binding agent to the extracellular space if the bispecific binding agent is internalized.

In another aspect, the present disclosure provides a multispecific binding agent comprising a bispecific antibody or antibody derivative, the bispecific binding agent comprising:
a) a first binding domain that specifically binds to an extracellular epitope of an EGFR protein of a target cell; and
b) a second binding domain that specifically binds to an extracellular epitope of a membrane-associated internalizing protein on a target cell;
   wherein the membrane associated internalizing protein is selected from CD205, CD166, SLC34A2, ITGB6, LRRC15, MUC16, SLC39A6, AXL, CD40, CD228, MUC5A, ITGB1, STn, KAAG1, DLK1, 5T4, SEZ6, ADAM9, I-Ag7, ENPP3, CD46, CD56, ROR1, GPR20, TM4SF1, B7-H4, ALPP, LY6E, CLDN18, LY6G6D, GPR56, CD71, LGR5, LY75, CD276/B7-H3, MST1R, MSLN, EpCAM, TNFRSF10B, STEAP1, MELTF, TROP2, CDH17, RNF43, and RNF128.

### Membrane-Associated Internalizing Proteins

Methods and multispecific binding agents of the present disclosure utilize membrane-associated internalizing proteins to cause internalization and/or degradation of the EGFR protein. The present disclosure utilizes the innate function of membrane-associated internalizing proteins to internalize upon binding of a binding agent to the protein. By simultaneously binding to EGFR using the first binding domain and binding to a membrane-associated internalizing proteins using the second binding domain, the multifunctional binding agent causes the EGFR protein to be internalized into the target cell with the membrane-associated internalizing protein. Once internalized, the EGFR protein will be sequestered and/or degraded (e.g., via lysosomal degradation) within the target cell.

Membrane-associated internalizing proteins for use in methods and bifunctional binding agents of the present disclosure include cell-surface protein that internalize upon binding of a binding agent (e.g., an antibody) to the protein. Such membrane-associate internalizing proteins include cell-surface proteins that are currently targeted by antibody-drug conjugates, which generally rely on internalization of the antibody-protein complex to ensure release of the conjugated drug. Examples of such membrane-associate internalizing proteins useful for methods of the present disclosure include, for example, CEACAM5 (i.e., CEA Cell Adhesion Molecule 5), CEACAM6 (i.e., CEA Cell Adhesion Molecule 6), HER3 (i.e., Receptor Tyrosine-Protein Kinase erbB-3), MUC1 (i.e., Mucin 1), CD205 (i.e., Lymphocyte Antigen 75), CD166 (i.e., Activated Leukocyte Cell Adhesion Molecule, also known as ALCAM)), PRLR (i.e., Prolactin Receptor), SLC34A2 (i.e., Solute Carrier Family 34 Member 2), ITGB6 (i.e., Integrin Subunit Beta 6), LRRC15 (i.e., Leucine-Rich Repeat-Containing Protein 15), and MUC16 (i.e., Mucin 16). It has been demonstrated that these proteins internalize into a cell upon binding of a binding agent (e.g., antibody) to an extracellular epitope of the protein.

In some embodiments, the membrane-associated internalizing protein is selected from SLC39A6 (i.e., Solute Carrier Family 39 Member 6), AXL (i.e., AXL Receptor Tyrosine Kinase, also known as Tyrosine-Protein Kinase Receptor UFO), CD40 (i.e., CD40 Molecule, also known as Tumor Necrosis Factor Receptor Superfamily Member 5), CD228 (i.e., Melanotransferrin), MUC5A (i.e, Mucin 5AC, Oligomeric Mucus/Gel-Forming), ITGB1 (i.e., Integrin Subunit Beta 1), STn, KAAG1 (i.e., Kidney Associated DCDC2 Antisense RNA 1), DLK1 (i.e., Delta Like Non-Canonical Notch Ligand 1), 5T4 (i.e., Trophoblast Glycoprotein), SEZ6 (i.e., Seizure Related 6 Homolog), ADAM9 (i.e., ADAM Metallopeptidase Domain 9), I-Ag7 (i.e., MHC Class II Molecule Ag7), ENPP3 (i.e., Ectonucleotide Pyrophosphatase/Phosphodiesterase 3), CD46 (i.e., CD46 Molecule), CD56 (i.e., Neural Cell Adhesion Molecule 1), ROR1 (i.e., Receptor Tyrosine Kinase Like Orphan Receptor 1), GPR20 (i.e., G Protein-Coupled Receptor 20), TM4SF1 (i.e., Transmembrane 4 L Size Family Member 1), B7-H4 (i.e., V-Set Domain Containing T Cell Activation Inhibitor 1), ALPP (i.e., Alkaline Phosphatase, Placental), LY6E (i.e., Lymphocyte Antigen 6 Family Member E), CLDN18 (i.e., Claudin 18), LY6G6D (i.e., Lymphocyte Antigen 6 Family Member G6D), GPR56 (i.e., Adhesion G Protein-Coupled Receptor GI), CD71 (Transferrin Receptor-1), B7-H3 (i.e., B7 homolog 3 protein or Cluster of Differentiation 276 or CD276), CDH17 (i.e., Cadherin 17), LGR5 (Leucine-rich repeat-containing G-protein coupled receptor 5), LY75 (Lymphocyte antigen 75 or CD205), MST1R (Macrophage stimulating 1 receptor), MSLN (Mesothelin), EpCAM (Epithelial Cell Adhesion Molecule), TNFRSF10B (TNF Receptor Superfamily Member 10b), STEAP1 (STEAP family member 1), MELTF (Melanotransferrin), TROP2 (Tumor associated calcium signal transducer 2), RNF43 (Ring finger protein 43), and RNF128 (Ring finger protein 128). It has been demonstrated that these proteins internalize into a cell upon binding of a binding agent (e.g., antibody) to an extracellular epitope of the protein.

In some embodiments, the membrane-associated internalizing protein is selected from CEACAM5, CEACAM6, HER3, MUC1, CD205, CD166, PRLR, SLC34A2, ITGB6, LRRC15, MUC16, SLC39A6, AXL, CD40, CD228, MUC5A, ITGB1, STn, KAAG1, DLK1, 5T4, SEZ6, ADAM9, I-AG7, ENPP3, CD46, CD56, ROR1, GPR20, TM4SF1, B7-H4, ALPP, LY6E, CLDN18, LY6G6D, GPR56, and CD71. In some embodiments, the membrane-associated internalizing protein is selected from CD205, CD166, SLC34A2, ITGB6, LRRC15, MUC16, SLC39A6, AXL, CD40, CD228, MUC5A, ITGB1, STn, KAAG1, DLK1, 5T4, SEZ6, ADAM9, I-AG7, ENPP3, CD46, CD56, ROR1, GPR20, TM4SF1, B7-H4, ALPP, LY6E, CLDN18, LY6G6D, GPR56, and CD71.

In some embodiments, the membrane-associated internalizing protein is selected from SLC34A2, ITGB6, LRRC15, MUC16, SLC39A6, AXL, CD40, CD228, MUC5A, ITGB1, STn, KAAG1, DLK1, 5T4, SEZ6, ADAM9, I-AG7, ENPP3, CD46, CD56, ROR1, GPR20, TM4SF1, B7-H4, ALPP, LY6E, CLDN18, LY6G6D, GPR56, and CD71. In some embodiments, the membrane-associated internalizing protein is selected from CD205, CD166, LRRC15, MUC16, SLC39A6, AXL, CD40, CD228, MUC5A, ITGB1, STn, KAAG1, DLK1, 5T4, SEZ6, ADAM9, I-AG7, ENPP3, CD46, CD56, ROR1, GPR20, TM4SF1, B7-H4, ALPP, LY6E, CLDN18, LY6G6D, GPR56, and CD71. In some embodiments, the membrane-associated internalizing protein is selected from CD205, CD166, SLC34A2, ITGB6, SLC39A6, AXL, CD40, CD228, MUC5A, ITGB1, STn, KAAG1, DLK1, 5T4, SEZ6, ADAM9, I-AG7, ENPP3, CD46, CD56, ROR1, GPR20, TM4SF1, B7-H4, ALPP, LY6E, CLDN18, LY6G6D, GPR56, and CD71.

In some embodiments, the membrane-associated internalizing protein is selected from SLC39A6, AXL, CD40, CD228, MUC5A, ITGB1, STn, KAAG1, DLK1, 5T4, SEZ6, ADAM9, I-AG7, ENPP3, CD46, CD56, ROR1, GPR20, TM4SF1, B7-H4, ALPP, LY6E, CLDN18, LY6G6D, GPR56, and CD71. In some embodiments, the membrane-associated internalizing protein is selected from CD228, MUC5A, ITGB1, STn, KAAG1, DLK1, 5T4, SEZ6, ADAM9, I-AG7, ENPP3, CD46, CD56, ROR1, GPR20, TM4SF1, B7-H4, ALPP, LY6E, CLDN18, LY6G6D, GPR56, and CD71.

In some embodiments, the membrane-associated internalizing protein is selected from SLC39A6, AXL, CD40, STn, KAAG1, DLK1, 5T4, SEZ6, ADAM9, I-AG7, ENPP3, CD46, CD56, ROR1, GPR20, TM4SF1, B7-H4, ALPP, LY6E, CLDN18, LY6G6D, GPR56, and CD71. In some embodiments, the membrane-associated internalizing protein is selected from SLC39A6, AXL, CD40, CD228, MUC5A, ITGB1, 5T4, SEZ6, ADAM9, I-AG7, ENPP3, CD46, CD56, ROR1, GPR20, TM4SF1, B7-H4, ALPP, LY6E, CLDN18, LY6G6D, GPR56, and CD71. In some embodiments, the membrane-associated internalizing protein is selected from SLC39A6, AXL, CD40, CD228, MUC5A, ITGB1, STn, KAAG1, DLK1, I-AG7, ENPP3, CD46, CD56, ROR1, GPR20, TM4SF1, B7-H4, ALPP, LY6E, CLDN18, LY6G6D, GPR56, and CD71.

In some embodiments, the membrane-associated internalizing protein is selected from SLC39A6, AXL, CD40, CD228, MUC5A, ITGB1, STn, KAAG1, DLK1, 5T4, SEZ6, ADAM9, CD56, ROR1, GPR20, TM4SF1, B7-H4, ALPP, LY6E, CLDN18, LY6G6D, GPR56, and CD71. In some embodiments, the membrane-associated internalizing protein is selected from SLC39A6, AXL, CD40, CD228, MUC5A, ITGB1, STn, KAAG1, DLK1, 5T4, SEZ6, ADAM9, I-AG7, ENPP3, CD46, TM4SF1, B7-H4, ALPP, LY6E, CLDN18, LY6G6D, GPR56, and CD71. In some embodiments, the membrane-associated internalizing protein is selected from SLC39A6, AXL, CD40, CD228, MUC5A, ITGB1, STn, KAAG1, DLK1, 5T4, SEZ6, ADAM9, I-AG7, ENPP3, CD46, CD56, ROR1, GPR20, LY6E, CLDN18, LY6G6D, GPR56, and CD71. In some embodiments, the membrane-associated internalizing protein is selected from SLC39A6, AXL, CD40, CD228, MUC5A, ITGB1, STn, KAAG1, DLK1, 5T4, SEZ6, ADAM9, I-AG7, ENPP3, CD46, CD56, ROR1, GPR20, TM4SF1, B7-H4, and ALPP.

In some embodiments, the membrane-associated internalizing protein is selected from CD205, CD166, CD40, CD228, CD46, CD56, and CD71. In some embodiments, the membrane-associated internalizing protein is selected from SLC34A2, ITGB6, LRRC15, MUC16, SLC39A6, AXL, MUC5A, ITGB1, STn, KAAG1, DLK1, 5T4, SEZ6, ADAM9, I-AG7, ENPP3, ROR1, GPR20, TM4SF1, B7-H4, ALPP, LY6E, CLDN18, LY6G6D, and GPR56.

In some embodiments, the membrane-associated internalizing protein is selected from CD205, CD166, SLC34A2, ITGB6, LRRC15, and MUC16. In some embodiments, the membrane-associated internalizing protein is selected from CD166, SLC34A2, ITGB6, LRRC15, and MUC16. In some embodiments, the membrane-associated internalizing protein is selected from CD205, SLC34A2, ITGB6, LRRC15, and MUC16. In some embodiments, the membrane-associated internalizing protein is selected from CD205, CD166, ITGB6, LRRC15, and MUC16. In some embodiments, the membrane-associated internalizing protein is selected from CD205, CD166, SLC34A2, LRRC15, and MUC16. In some embodiments, the membrane-associated internalizing protein is selected from CD205, CD166, SLC34A2, ITGB6, and MUC16. In some embodiments, the membrane-associated internalizing protein is selected from CD205, CD166, SLC34A2, ITGB6, and LRRC15.

In some embodiments, the membrane-associated internalizing protein is selected from B7-H3, CDH17, MUC1, ITGB6, RNF43, and CECAM5. In some embodiments, the membrane-associated internalizing protein is selected from B7-H3, CDH17, MUC1, ITGB6, and CECAM5.

In some embodiments, the membrane-associated internalizing protein is selected from CDH17, MUC1, ITGB6, and CECAM5. In some embodiments, the membrane-associated internalizing protein is selected from B7-H3, MUC1, ITGB6, and CECAM5. In some embodiments, the membrane-associated internalizing protein is selected from B7-H3, CDH17, ITGB6, and CECAM5. In some embodiments, the membrane-associated internalizing protein is selected from B7-H3, CDH17, MUC1, and CECAM5. In some embodiments, the membrane-associated internalizing protein is selected from MUC1, ITGB6, and CECAM5. In some embodiments, the membrane-associated internalizing protein is selected from CD166, SLC34A2, ITGB6, LRRC15, and MUC16. In some embodiments, the membrane-associated internalizing protein is selected from SLC34A2, ITGB6, LRRC15, and MUC16. In some embodiments, the membrane-associated internalizing protein is selected from CD166, ITGB6, LRRC15, and MUC16. In some embodiments, the membrane-associated internalizing protein is selected from CD166, SLC34A2, LRRC15, and MUC16. In some embodiments, the membrane-associated internalizing protein is selected from CD166, SLC34A2, ITGB6, and MUC16. In some embodiments, the membrane-associated internalizing protein is selected from CD166, SLC34A2, ITGB6, and LRRC15.

In some embodiments, the membrane-associated internalizing protein is selected from, SLC34A2, ITGB6, LRRC15, and MUC16. In some embodiments, the membrane-associated internalizing protein is selected from CD205, ITGB6, LRRC15, and MUC16. In some embodiments, the membrane-associated internalizing protein is selected from CD205, SLC34A2, LRRC15, and MUC16. In some embodiments, the membrane-associated internalizing protein is selected from CD205, SLC34A2, ITGB6, and MUC16. In some embodiments, the membrane-associated internalizing protein is selected from CD205, SLC34A2, ITGB6, and LRRC15.

In some embodiments, the membrane-associated internalizing protein is selected from CD166, ITGB6, LRRC15, and MUC16. In some embodiments, the membrane-associated internalizing protein is selected from CD205, ITGB6, LRRC15, and MUC16. In some embodiments, the membrane-associated internalizing protein is selected from CD205, CD166, ITGB6, LRRC15, and MUC16. In some embodiments, the membrane-associated internalizing protein is selected from CD205, CD166, LRRC15, and MUC16. In some embodiments, the membrane-associated internalizing protein is selected from CD205, CD166, ITGB6, and MUC16. In some embodiments, the membrane-associated internalizing protein is selected from CD205, CD166, ITGB6, and LRRC15.

In some embodiments, the membrane-associated internalizing protein is selected from CD166, SLC34A2, LRRC15, and MUC16. In some embodiments, the membrane-associated internalizing protein is selected from CD205, SLC34A2, LRRC15, and MUC16. In some embodiments, the membrane-associated internalizing protein is selected from CD205, CD166, LRRC15, and MUC16. In some embodiments, the membrane-associated internalizing protein is selected from CD205, CD166, SLC34A2, LRRC15, and MUC16. In some embodiments, the membrane-associated internalizing protein is selected from CD205, CD166, SLC34A2, and MUC16. In some embodiments, the membrane-associated internalizing protein is selected from CD205, CD166, SLC34A2, and LRRC15.

In some embodiments, the membrane-associated internalizing protein is selected from CD166, SLC34A2, ITGB6, and MUC16. In some embodiments, the membrane-associated internalizing protein is selected from CD205, SLC34A2, ITGB6, and MUC16. In some embodiments, the membrane-associated internalizing protein is selected from CD205, CD166, ITGB6, and MUC16. In some embodiments, the membrane-associated internalizing protein is selected from CD205, CD166, SLC34A2, and MUC16. In some embodiments, the membrane-associated internalizing protein is selected from CD205, CD166, SLC34A2, ITGB6, and MUC16. In some embodiments, the membrane-associated internalizing protein is selected from CD205, CD166, SLC34A2, and ITGB6.

In some embodiments, the membrane-associated internalizing protein is selected from CD166, SLC34A2, ITGB6, and LRRC15. In some embodiments, the membrane-associated internalizing protein is selected from CD205, SLC34A2, ITGB6, and LRRC15. In some embodiments, the membrane-associated internalizing protein is selected from CD205, CD166, ITGB6, and LRRC15. In some embodiments, the membrane-associated internalizing protein is selected from CD205, CD166, SLC34A2, and LRRC15. In some embodiments, the membrane-associated internalizing protein is selected from CD205, CD166, SLC34A2, and ITGB6.

In some embodiments, the membrane associated internalizing protein is CD205 or CD166. In some embodiments, the membrane associated internalizing protein is CD205 or SLC34A2. In some embodiments, the membrane associated internalizing protein is CD205 or ITGB6. In some embodiments, the membrane associated internalizing protein is CD205 or LRRC15. In some embodiments, the membrane associated internalizing protein is CD205 or MUC16.

In some embodiments, the membrane associated internalizing protein is CD166 or CD205. In some embodiments, the membrane associated internalizing protein is CD166 or SLC34A2. In some embodiments, the membrane associated internalizing protein is CD166 or ITGB6. In some embodiments, the membrane associated internalizing protein is CD166 or LRRC15. In some embodiments, the membrane associated internalizing protein is CD166 or MUC16.

In some embodiments, the membrane associated internalizing protein is SLC34A2 or CD205. In some embodiments, the membrane associated internalizing protein is SLC34A2 or CD166. In some embodiments, the membrane associated internalizing protein is SLC34A2 or ITGB6. In some embodiments, the membrane associated internalizing protein is SLC34A2 or LRRC15. In some embodiments, the membrane associated internalizing protein is SLC34A2 or MUC16.

In some embodiments, the membrane associated internalizing protein is ITGB6 or CD205. In some embodiments, the membrane associated internalizing protein is ITGB6 or CD166. In some embodiments, the membrane associated internalizing protein is ITGB6 or SLC34A2. In some embodiments, the membrane associated internalizing protein is ITGB6 or LRRC15. In some embodiments, the membrane associated internalizing protein is ITGB6 or MUC16.

In some embodiments, the membrane associated internalizing protein is LRRC15 or CD205. In some embodiments, the membrane associated internalizing protein is LRRC15 or CD166. In some embodiments, the membrane associated internalizing protein is LRRC15 or SLC34A2. In some embodiments, the membrane associated internalizing protein is LRRC15 or ITGB6. In some embodiments, the membrane associated internalizing protein is LRRC15 or MUC16.

In some embodiments, the membrane associated internalizing protein is MUC16 or CD205. In some embodiments, the membrane associated internalizing protein is MUC16 or CD166. In some embodiments, the membrane associated internalizing protein is MUC16 or SLC34A2. In some embodiments, the membrane associated internalizing protein is MUC16 or ITGB6. In some embodiments, the membrane associated internalizing protein is MUC16 or LRRC15.

In some embodiments, the membrane associated internalizing protein is CD205. In some embodiments, the membrane associated internalizing protein is CD166. In some embodiments, the membrane associated internalizing protein is SLC34A2. In some embodiments, the membrane associated internalizing protein is ITGB6. In some embodiments, the membrane associated internalizing protein is LRRC15. In some embodiments, the membrane associated internalizing protein is MUC16.

In some embodiments, the membrane associated internalizing protein is CEACAM5. In some embodiments, the membrane associated internalizing protein is CEACAM6. In some embodiments, the membrane associated internalizing protein is HER3. In some embodiments, the membrane associated internalizing protein is MUC1. In some embodiments, the membrane associated internalizing protein is CD205. In some embodiments, the membrane associated internalizing protein is CD166. In some embodiments, the membrane associated internalizing protein is PRLR. In some embodiments, the membrane associated internalizing protein is SLC34A2. In some embodiments, the membrane associated internalizing protein is ITGB6. In some embodiments, the membrane associated internalizing protein is LRRC15. In some embodiments, the membrane associated internalizing protein is MUC16.

In some embodiments, the membrane-associated internalizing protein is selected from SLC39A6. In some embodiments, the membrane-associated internalizing protein is selected from AXL. In some embodiments, the membrane-associated internalizing protein is selected from CD40. In some embodiments, the membrane-associated internalizing protein is selected from CD228. In some embodiments, the membrane-associated internalizing protein is selected from MUC5A. In some embodiments, the membrane-associated internalizing protein is selected from ITGB1. In some embodiments, the membrane-associated internalizing protein is selected from STn. In some embodiments, the membrane-associated internalizing protein is selected from KAAG1.

In some embodiments, the membrane-associated internalizing protein is selected from DLK1. In some embodiments, the membrane-associated internalizing protein is selected from 5T4. In some embodiments, the membrane-associated internalizing protein is selected from SEZ6. In some embodiments, the membrane-associated internalizing protein is selected from ADAM9. In some embodiments, the membrane-associated internalizing protein is selected from I-AG7. In some embodiments, the membrane-associated internalizing protein is selected from ENPP3. In some embodiments, the membrane-associated internalizing protein is selected from CD46. In some embodiments, the membrane-associated internalizing protein is selected from CD56. In some embodiments, the membrane-associated internalizing protein is selected from ROR1.

In some embodiments, the membrane-associated internalizing protein is selected from GPR20. In some embodiments, the membrane-associated internalizing protein is selected from TM4SF1. In some embodiments, the membrane-associated internalizing protein is selected from B7-H4. In some embodiments, the membrane-associated internalizing protein is selected from ALPP. In some embodiments, the membrane-associated internalizing protein is selected from LY6E. In some embodiments, the membrane-associated internalizing protein is selected from CLDN18. In some embodiments, the membrane-associated internalizing protein is selected from LY6G6D. In some embodiments, the membrane-associated internalizing protein is selected from GPR56. In some embodiments, the membrane-associated internalizing protein is selected from CD71.

In some embodiments, the membrane associated internalizing protein is LGR5. In some embodiments, the membrane associated internalizing protein is LY75. In some embodiments, the membrane associated internalizing protein is CD276/B7-H3. In some embodiments, the membrane associated internalizing protein is MST1R. In some embodiments, the membrane associated internalizing protein is MSLN. In some embodiments, the membrane associated internalizing protein is EpCAM. In some embodiments, the membrane associated internalizing protein is TNFRSF10B. In some embodiments, the membrane associated internalizing protein is TEAP1. In some embodiments, the membrane associated internalizing protein is MELTF. In some embodiments, the membrane associated internalizing protein is TROP2. In some embodiments, the membrane associated internalizing protein is CDH17. In some embodiments, the membrane associated internalizing protein is RNF43. In some embodiments, the membrane associated internalizing protein is RNF43.

In some embodiments, the membrane-associated internalizing protein is recycled to the target cell surface following the internalization of the binding agent. In some embodiments, the membrane-associated internalizing protein is degraded.

### Membrane-Associated Degrading Proteins

Methods and multispecific binding agents of the present disclosure may utilize membrane-associated degrading proteins to cause degradation of the EGFR protein. The present disclosure may use the membrane-associated degrading proteins to cause ubiquitination upon binding of a binding agent to the membrane-associated degrading protein. By also binding to EGFR at the first binding domain and binding to a membrane-associated degrading proteins using the second binding domain, the multifunctional binding agent can cause the EGFR protein to be degraded with the membrane-associated degrading protein.

Membrane-associated degrading proteins for use in methods and bifunctional binding agents of the present disclosure can include a cell-surface protein that is degraded upon binding and/or internalization of a binding agent (e.g., an antibody) to the protein. Such membrane-associated degrading proteins can include cell-surface proteins that are targeted by antibody-drug conjugates, which can rely on degradation of the antibody-protein complex to ensure release of the conjugated drug. Examples of such membrane-associate degrading proteins useful for methods of the present disclosure can include, for example, TROP2. In some embodiments, the membrane-associated degrading protein is an E3 ligase. In some embodiments, the membrane-associated degrading protein is RNF43 (i.e., Ring Finger Protein 43).

### First Binding Region

In some embodiments, the first binding domain is derived from an antibody directed at a membrane associated internalizing protein or a degrading protein. Such antibodies are known to those skilled in the art and can be incorporated into methods and bispecific binding agents of the present disclosure. For example, in some embodiments, the complementarity-determining regions (“CDR”) of known antibodies directed at the membrane associated internalizing protein of interest or the membrane associated degrading protein of interest can be incorporated into multispecific binding agents and methods of the present disclosure using known techniques. Exemplary antibodies suitable for incorporation into the methods and multispecific binding agents of the present disclosure include those described below.

For example, antibodies targeting CEACAM 5 are known in the art, including, for example the CC4 antibody disclosed in, for example, Zheng, Chaogu, et al., “A novel anti-CEACAM5 monoclonal antibody, CC4, suppresses colorectal tumor growth and enhances NK cells-mediated tumor immunity.” PloS one 6.6 (2011): e21146. Additional antibodies targeting CEACAM5 that are suitable for use in the present disclosure include, for example, the anti-CEACAM5 antibodies MN-14, MN-15, and MN-3, described, for example, in Blumenthal, Rosalyn D., Hans J. Hansen, and David M. Goldenberg. “Inhibition of adhesion, invasion, and metastasis by antibodies targeting CEACAM6 (NCA-90) and CEACAM5 (Carcinoembryonic Antigen).” Cancer research 65.19 (2005): 8809-8817.

Antibodies targeting CEACAM6 are known in the art, including, for example, the anti-CEACAM6 antibodies sdAb, 2Ab, 4Ab described, for example in Wu, Shang-Jung, et al. “Migration and invasion of NSCLC suppressed by the downregulation of Src/focal adhesion kinase using single, double and tetra domain anti-CEACAM6 antibodies.” Translational oncology 14.7 (2021): 101057. Additional antibodies targeting CEACAM6 that are suitable for use in the present disclosure include, for example, the anti-CEACAM6 antibodies MN-3 and MN-15 as described, for example, in Blumenthal, Rosalyn D., Hans J. Hansen, and David M. Goldenberg. “Inhibition of adhesion, invasion, and metastasis by antibodies targeting CEACAM6 (NCA-90) and CEACAM5 (Carcinoembryonic Antigen).” Cancer research 65.19 (2005): 8809-8817.

Antibodies targeting HER3 (also known as ErbB-3) are known in the art, including, for example, the anti-HER3 antibody GSK2849330 described, for example, in Gan, Hui K., et al. “A phase I, first-in-human study of GSK2849330, an anti-HER3 monoclonal antibody, in HER3-expressing solid tumors.” The oncologist 26.10 (2021): e1844-e1853. Further anti-HER3 antibodies include, for example, Patritumab (U3-1287), which is described in, for example, Hashimoto, Yuuri, et al. “A Novel HER3-Targeting Antibody-Drug Conjugate, U3-1402, Exhibits Potent Therapeutic Efficacy through the Delivery of Cytotoxic Payload by Efficient Internalization Preclinical Evaluation of U3-1402, a HER3-Targeting ADC.” Clinical Cancer Research 25.23 (2019): 7151-7161.

Antibodies targeting MUC1 are known in the art, for example, including, the anti-MUC1 antibodies MY.1E12, KL6, 5E5, and TAB004 described in Bose, Mukulika, and Pinku Mukherjee. “Potential of anti-MUC1 antibodies as a targeted therapy for gastrointestinal cancers.” Vaccines 8.4 (2020): 659.

Antibodies targeting CD205 are known in the art, including, for example, the anti-CD205 antibody MEN1309/0BT076 described, for example, in Rieke, Damian T., and Ulrich Keller. “A CD205-directed antibody drug conjugate-lymphoma precision oncology or sophisticated chemotherapy?” Haematologica 105.11 (2020): 2504.

Antibodies targeting CD166 are known in the art, for example, the anti-CD166 antibody CX-2009 described in, for example, Boni, Valentina, et al. “Praluzatamab ravtansine, a CD166-targeting antibody-drug conjugate, in patients with advanced solid tumors: an open-label phase 1/2 trial of Praluzatamab ravtansine in patients with advanced tumors.” Clinical Cancer Research (2022).

Antibodies targeting PRLR are known in the art, for example, the anti-PRLR antibody ABBV-176 described in, for example, Anderson, Mark G., et al. “ABBV-176, a PRLR antibody drug conjugate with a potent DNA-damaging PBD cytotoxin and enhanced activity with PARP inhibition.” BMC cancer 21.1 (2021): 1-11.]). Additional antibodies targeting CEACAM6 that are suitable for use in the present disclosure include, for example, the anti-CEACAM6 antibody LFA102 described in Damiano, Jason S., et al. “Neutralization of Prolactin Receptor Function by Monoclonal Antibody LFA102, a Novel Potential Therapeutic for the Treatment of Breast Cancer Preclinical Development of Anti-PRLR Antibody LFA102.” Molecular cancer therapeutics 12.3 (2013): 295-305.

Antibodies targeting SCL34A2 are known in the art, for example the anti-NaPi2b antibody described in Lin, Kedan, et al. “Preclinical Development of an Anti-NaPi2b (SLC34A2) Antibody-Drug Conjugate as a Therapeutic for Non-Small Cell Lung and Ovarian Cancers Preclinical Development of NaPi2b Antibody-Drug Conjugate.” Clinical Cancer Research 21.22 (2015): 5139-5150. Another antibody suitable for incorporation into bispecific binding agents of the present disclosure include the anti-SCL34A2 antibody MX35 described in Yin, Beatrice W T, et al. “Monoclonal antibody MX35 detects the membrane transporter NaPi2b (SLC34A2) in human carcinomas.” Cancer immunity 8.1 (2008).

Antibodies targeting ITGB6 are known in the art, including, for example the antibody SGN-B6A described in, for example, Patnaik, Amita, et al. “A phase 1 study of SGN-B6A, an antibody-drug conjugate targeting integrin beta-6, in patients with advanced solid tumors (SGN-B6A-001, Trial in Progress).” (2021). Another antibody suitable for incorporation into the present disclosure include the anti-ITGB6 antibodies TPS3144-TPS3144 described in Zheng, Xiaoxia, et al. “Silencing of ITGB6 inhibits the progression of cervical carcinoma via regulating JAK/STAT3 signaling pathway.” Annals of Translational Medicine 9.9 (2021).

Antibodies targeting LRRC15 are known in the art, including for example, the anti-LRCC15 antibody ABBV-085 described in, for example, Demetri, George D., et al. “First-in-Human Phase I Study of ABBV-085, an Antibody-Drug Conjugate Targeting LRRC15, in Sarcomas and Other Advanced Solid Tumors Phase I Study of ABBV-085, an LRRC15-Targeting ADC.” Clinical Cancer Research 27.13 (2021): 3556-3566; and Slemmons, Katherine K., et al. “LRRC15 antibody-drug conjugates show promise as osteosarcoma therapeutics in preclinical studies.” Pediatric blood & cancer 68.2 (2021): e28771]).

Antibodies targeting MUC16 are known in the art, including, for example, the anti-MUC16 antibody OC125 described in, for example, Rao, Thapi Dharma, et al. “Novel monoclonal antibodies against the proximal (carboxy-terminal) portions of MUC16.” Applied immunohistochemistry & molecular morphology: AIMM/official publication of the Society for Applied Immunohistochemistry 18.5 (2010): 462. Additional anti-MUC16 antibodies include, for example, those described in Aithal, Abhijit, et al. “MUC16 as a novel target for cancer therapy.” Expert opinion on therapeutic targets 22.8 (2018): 675-686; and Rao, Thapi Dharma, et al. “Antibodies against specific MUC16 glycosylation sites inhibit ovarian cancer growth.” ACS chemical biology 12.8 (2017): 2085-2096]).

Antibodies targeting SLC39A6 are known in the art, including, for example, the anti-SLC39A6 antibody described in Cui, Shen, et al., “SLC39A6: a potential target for diagnosis and therapy of esophageal carcinoma.” Journal of Translational Medicine 13 (2015): 321. Additional anti-SLC29A6 antibodies include, for example, those described in Sussman, Smith, et al. “SGN-LIV1A: A novel antibody-drug conjugate targeting LIV-1 for the treatment of metastatic breast cancer.” Mol Chancer Ther (2014) 13 (12): 2991-3000; and Wan and Wang “Role of SLC39A in the development and progression of liver cancer.” Oncology Letters 23.3. (2022): 77.

Antibodies targeting AXL are known in the art, including, for example, the AXL-specific antibody described in Vajkoczy, Knyazev, et al. “Dominant-negative inhibition of the Axl receptor tyrosine kinase suppresses brain tumor cell growth and invasion and prolongs survival.” Proceedings of the National Academy of Sciences 103.15 (2006): 5799-5804. An additional anti-AXL antibody includes, for example, the anti-AXL antibody 20G7-D9 described in Leconet, Chentouf, et al. “Therapeutic activity of anti-AXL antibody against triple-negative breast caser patient-derived xenografts and metastasis.” Clin Cancer Research 23.11 (2017):2806-2816.

Antibodies targeting CD40 are known in the art, including, for example, are known in the art, including, for example, the anti-CD40 antibody described in Xu, Gao, et al. “Repulsive guidance molecule a blockade exerts the immunoregulatory function in DCs stimulated with ABP and LPS.” Human vaccines & immunotherapeutics 12.8 (2016): 2169-2180. Additional anti-CD40 antibodies include, for example, those described in Silvin, Chapuis, et al. “Elevated calprotectin and abnormal myeloid cell subsets discriminate severe from mild COVID-19.” Cell 182.6 (2020): 1401-1418; and in Ceglia, Zurawski, et al. “Anti-CD40 Antibody Fused to CD40 Ligand Is a Superagonist Platform for Adjuvant Intrinsic DC-Targeting Vaccines.” Frontiers in immunology 12:786144 (2021).

Antibodies targeting CD228 are known in the art, including, for example, the anti-MELTF antibody described in Sawaki, Kanda, et al. “Level of melanotransferrin in tissue and sera serves as a prognostic marker of gastric cancer.” Anticancer Research 39.11 (2019): 6125-6133. An additional anti-CD228 antibody includes, for example, that described in Singh, Eyford, et al. “Discovery of a Highly Conserved Peptide in the Iron Transporter Melanotransferrin that Traverses an Intact Blood Brain Barrier and Localizes in Neural Cells.” Frontiers in neuroscience 15: 596976. (2021): 473.

Antibodies targeting MUC5A are known in the art, including, for example, the anti-MUC5A antibody MUC5:TR-3A described in Zuhdi Alimam, Piazza, et al. “Muc-5/5ac mucin messenger RNA and protein expression is a marker of goblet cell metaplasia in murine airways.” American journal of respiratory cell and molecular biology 22.3 (2000): 253-260. Additional anti-MUC5 antibodies include, for example, those described in Wang, Jin, et al. “Expression of survivin, MUC2 and MUC5 in colorectal cancer and their association with clinicopathological characteristics.” Oncology Letters 14.1 (2017): 1011-1016; and in Reis, David, et al. “Immunohistochemical study of MUC5AC expression in human gastric carcinomas using a novel monoclonal antibody.” International journal of cancer 74.1 (1997): 112-121.

Antibodies targeting ITGB1 are known in the art, including, for example, the anti-ITGB1 antibody described in Du, Yang, et al. “The circular RNA circSKA3 binds integrin β1 to induce invadopodium formation enhancing breast cancer invasion.” Molecular Therapy 28.5 (2020): 1287-1298. Additional anti-ITGB1 antibodies include, for example, those described in Kawahara, Niwa, et al. “Integrin 01 is an essential factor in vasculogenic mimicry of human cancer cells.” Cancer science 109.8 (2018): 2490-2496; and in Wang and Li. “Ropivacaine inhibits the proliferation and migration of colorectal cancer cells through ITGB1.” Bioengineered 12.1 (2021): 44-53.

Antibodies targeting STn are known in the art, including, for example, the anti-STn antibody described in Prendergast, da Silva, et al. “Novel anti-Sialyl-Tn monoclonal antibodies and antibody-drug conjugates demonstrate tumor specificity and anti-tumor activity.” mAbs 9,4 (2017): 615-627. An additional anti-STn antibody includes, for example, that described in Eavarone, David A et al. “Humanized anti-Sialyl-Tn antibodies for the treatment of ovarian carcinoma.” PloS one 13,7 (2018) e0201314.27.

Antibodies targeting KAAG1 are known in the art, including, for example, the anti-KAAG1 antibody anti-KAAG1 AB-3A described in U.S. Pat. No. 9,393,302 B2.

Antibodies targeting DLK1 are known in the art, including, for example, the anti-DLK1 antibody anti-DLK1 SIP(EB3) described in Bujak, Ritz, et al. “A monoclonal antibody to human Dlk1 reveals differential expression in cancer and absence in healthy tissues.” Antibodies 4.2 (2015): 71-87. Additional anti-DLKL antibodies include, for example, those described in Takagi, Zhao, et al. “Delta-like 1 homolog (DLK1) as a possible therapeutic target and its application to radioimmunotherapy using 1251-labelled anti-DLK1 antibody in lung cancer models (HOT1801 and FIGHT004).” Lung Cancer 153 (2021): 134-142; and in Huang, Zhang, et al. “Up-regulation of DLK1 as an imprinted gene could contribute to human hepatocellular carcinoma.” Carcinogenesis 28.5 (2007): 1094-1103.

Antibodies targeting 5T4 are known in the art, including, for example, the anti-5T4 antibody anti-5T4 IgGI described in Shapiro, Vaishampayan, et al. “First-in-human trial of an anti-5T4 antibody-monomethylauristatin conjugate, PF-06263507, in patients with advanced solid tumors.” Investigational New Drugs 35.3 (2017): 315-323. An additional anti-5T4 antibody includes, for example, that described in Owens, Sheard, et al. “Preclinical assessment of CAR T-cell therapy targeting the tumor antigen 5T4 in ovarian cancer.” Journal of Immunotherapy 41.3 (2018): 130-140.

Antibodies targeting SEZ6 are known in the art, including, for example, the anti-SEZ6 antibody described in Jiang, Chen, et al. “Correlation between human seizure-related gene 6 variants and idiopathic generalized epilepsy in a Southern Chinese Han population.” Neural Regeneration Research 7.2 (2012): 96-100. An additional anti-SEZ6 antibody includes, for example, that described in Kuhn, Koroniak, et al. “Secretome protein enrichment identifies physiological BACE1 protease substrates in neurons.” The EMBO journal 31.14 (2012): 3157-3168.

Antibodies targeting ADAM9 are known in the art, including, for example, the anti-ADAM9 antibody described in Mazzocca, Coppari, et al. “A secreted form of ADAM9 promotes carcinoma invasion through tumor-stromal interactions.” Cancer research 65.11 (2005): 4728-4738. Additional anti-ADAM9 antibodies include, for example, those described in Zigrino, Mauch, et al. “Adam-9 expression and regulation in human skin melanoma and melanoma cell lines.” International journal of cancer 116.6 (2005): 853-859; and in Kim, Jeung, et al. “The Effect of Disintegrin-Metalloproteinase ADAM9 in Gastric Cancer Progression.” Molecular cancer therapeutics 13.12 (2014): 3074-3085.

Antibodies targeting I-Ag7 are known in the art, including, for example, the anti-I-Ag7 antibody described in Zhang, Crawford, et al. “Monoclonal antibody blocking the recognition of an insulin peptide-MHC complex modulates type 1 diabetes.” Proceedings of the National Academy of Sciences 111.7 (2014): 2656-2661. Additional antibodies targeting I-Ag7 include, for example, those described in Noorchashm, Hooman, et al. “I-Ag7-mediated antigen presentation by B lymphocytes is critical in overcoming a checkpoint in T cell tolerance to islet R cells of nonobese diabetic mice.” The Journal of Immunology 163.2 (1999): 743-750.; and in Gardiner, Richards, et al. “Conformation of MHC class II I-Ag7 is sensitive to the P9 anchor amino acid in bound peptide.” International immunology 19.9 (2007): 1103-1113.

Antibodies targeting ENPP3 are known in the art, including, for example, the anti-ENPP3 antibody described in Boggavarapu, Lalitkumar, et al. “Compartmentalized gene expression profiling of receptive endometrium reveals progesterone regulated ENPP3 is differentially expressed and secreted in glycosylated form.” Scientific reports 6.1 (2016): 1-13. An additional anti-ENPP3 antibody includes, for example, that is described in Schiechl, Hermann, et al. “Basophils trigger fibroblast activation in cardiac allograft fibrosis development.” American Journal of Transplantation 16.9 (2016): 2574-2588.

Antibodies targeting CD46 are known in the art, including, for example, the anti-CD46 antibody anti-CD46 antibody YS5 described in Su, Liu, et al. “Targeting CD46 for both adenocarcinoma and neuroendocrine prostate cancer.” JCI insight 3.17 (2018) e121497. Additional anti-CD46 antibodies include, for example, those described in Carver-Ward, Hollanders, et al. “Progesterone does not potentiate the acrosome reaction in human spermatozoa: flow cytometric analysis using CD46 antibody.” Human reproduction 11.1 (1996): 121-126; and in Krey, Himmelreich, et al. “Function of bovine CD46 as a cellular receptor for bovine viral diarrhea virus is determined by complement control protein 1.” Journal of virology 80.8 (2006): 3912-3922.

Antibodies targeting CD56 are known in the art, including, for example, the anti-CD56 antibody described in Silvin, Chapuis, et al. “Elevated calprotectin and abnormal myeloid cell subsets discriminate severe from mild COVID-19.” Cell 182.6 (2020): 1401-1418. Additional anti-CD46 antibodies include, for example, those described in Zhan, Guo, et al. “Glioma stem-like cells evade interferon suppression through MBD3/NuRD complex-mediated STAT1 downregulation.” The Journal of experimental medicine 217,5 (2020): e20191340; and in Feng, Wang et al. “Differential killing of CD56-expressing cells by drug-conjugated human antibodies targeting membrane-distal and membrane-proximal non-overlapping epitopes.” mAbs 8.4 (2016): 799-810.

Antibodies targeting ROR1 are known in the art, including, for example, the anti-ROR1 antibody anti-ROR1 4A5 described in Balakrishnan, Goodpaster, et al. “Analysis of ROR1 Protein Expression in Human Cancer and Normal Tissues.” Clinical Cancer Research 23.12 (2017): 3061-3071. Additional anti-ROR1 antibodies include, for example, those described in Baskar, Wiestner et al. “Targeting malignant B cells with an immunotoxin against ROR1.” mAbs. 4.3 (2012) 349-361; and in Zhang, Chen et al. “ROR1 is expressed in human breast cancer and associated with enhanced tumor-cell growth.” PloS one 7,3 (2012): e31127.

Antibodies targeting GPR20 are known in the art, including, for example, the anti-GPR20 antibody described in Wheway, Schmidts, et al. “An siRNA-based functional genomics screen for the identification of regulators of ciliogenesis and ciliopathy genes.” Nature cell biology 17,8 (2015): 1074-1087. An additional anti-GPR20 antibody includes, for example, that described in Iida, Ahmed, et al. “Identification and Therapeutic Targeting of GPR20, Selectively Expressed in Gastrointestinal Stromal Tumors, with DS-6157a, a First-in-Class Antibody-Drug Conjugate.” Cancer Discovery 11.6 (2021): 1508-1523.

Antibodies targeting TM4SF1 are known in the art, including, for example, the anti-TM4SF1 antibody described in Zacharias, Frank, et al. “Regeneration of the lung alveolus by an evolutionarily conserved epithelial progenitor.” Nature 555,7695 (2018): 251-255. Additional antibodies targeting TM4SF1 include, for example, the anti-TM4SF1 antibody 8G4 described in Lin, Merley, et al. “TM4SF1: a new vascular t9herapeutic target in cancer.” Angiogenesis 17,4 (2014): 897-907; and the anti-TM4SF1 antibody described in Wang, Sun, et al. “B7-H3 suppresses doxorubicin-induced senescence-like growth arrest in colorectal cancer through the AKT/TM4SF1/SIRT1 pathway” Cell death & disease 12,5 (2021): 453.

Antibodies targeting B7-H4 are known in the art, including, for example, the anti-B7-H4 antibody described in Podojil, Glaser, et al. “Antibody targeting of B7-H4 enhances the immune response in urothelial carcinoma.” Oncoimmunology 9,1 (2020): 1744897. Additional antibodies targeting B7-H4 include, for example, those described in Miao and Sun. “Development of a novel anti-B7-H4 antibody enhances anti-tumor immune response of human T cells.” Biomedicine & pharmacotherapy 141 (2021): 111913; and in Dangaj, Lanitis, et al. “Novel Recombinant Human B7-H4 Antibodies Overcome Tumoral Immune Escape to Potentiate T-Cell Antitumor Responses Overcoming B7-H4-Mediated T-Cell Inhibition.” Cancer research 73.15 (2013): 4820-4829.

Antibodies targeting ALPP are known in the art, including, for example, the anti-ALPP antibody anti-ALPP SP15 described in Zwolanek, Satue, et al. “Tracking mesenchymal stem cell contributions to regeneration in an immunocompetent cartilage regeneration model.” JCI insight 2.20 (2017) e87322. Additional antibodies targeting ALPP include, for example, those described in Chen, Chen, et al. “Placental alkaline phosphatase promotes Zika virus replication by stabilizing viral proteins through BIP.” MBio 11.5 (2020): e01716-20; and in Odörfer, Egerbacher, et al. “Hematopoietic bone marrow cells participate in endothelial, but not epithelial or mesenchymal cell renewal in adult rats.” Journal of cellular and molecular medicine 15.10 (2011): 2232-2244.

Antibodies targeting LY6E are known in the art, including, for example, the anti-LY6E antibody described in Mar, Rinkenberger, et al. “LY6E mediates an evolutionarily conserved enhancement of virus infection by targeting a late entry step.” Nature communications 9.1 (2018): 1-14. Additional antibodies targeting LY6E include, for example, the anti-LY6E antibody anti-LY6E MTS35 described in Langford, Outhwaite, et al. “Deletion of the Syncytin A receptor Ly6e impairs syncytiotrophoblast fusion and placental morphogenesis causing embryonic lethality in mice.” Scientific reports 8,1 (2018): 3961; and the anti-LY6E antibody anti-LY6E 9B12 described in Dela Cruz Chuh, Josefa, et al. “Preclinical optimization of Ly6E-targeted ADCs for increased durability and efficacy of anti-tumor response.” MAbs 13.1 (2021).

Antibodies targeting CLDN18 are known in the art, including, for example, the anti-CLDN18 antibody described in Tureci, Mitnacht-Kraus, et al. “Characterization of zolbetuximab in pancreatic cancer models.” Oncoimmunology 8.1 (2019): e1523096. An additional anti-CLDN18 antibody includes, for example, that described in Matsusaka, Ushiku, et al. “Coupling CDH17 and CLDN18 markers for comprehensive membrane-targeted detection of human gastric cancer.” Oncotarget 7,39 (2016): 64168-64181.

Antibodies targeting LY6G6D are known in the art, including, for example, the anti-LY6G6D antibody described in Sewda, Coppola, et al. “Cell-surface markers for colon adenoma and adenocarcinoma.” Oncotarget 7,14 (2016): 17773-89. Additional anti-LY6G6D antibodies include, for example, the anti-LY6G6D antibody anti-LY6G6D clone 10C1 described in Corrales, Hipp, et al. “LY6G6D is a selectively expressed colorectal cancer antigen that can be used for targeting a therapeutic T-cell response by a T-cell engager. Frontiers in immunology 13 (2022): 1008764; and the anti-LY6G6D antibody described in Wang, Sun, et al. “Novel Anti-LY6G6D/CD3 T Cell-Dependent Bispecific Antibody for the Treatment of Colorectal Cancer.” Molecular Cancer Therapeutics 21:6 (2022): 974-985.

Antibodies targeting GPR56 are known in the art, including, for example, the anti-GPR56 antibody anti-GPR56 10C7 described in Chatterjee, Zhang, et al. “Anti-GPR56 monoclonal antibody potentiates GPR56-mediated Src-Fak signaling to modulate cell adhesion.” Journal of Biological Chemistry 296 (2021) 100261. Additional anti-GPR56 antibodies include, for example, those described in Iguchi, Sakata, et al. “Orphan G protein-coupled receptor GPR56 regulates neural progenitor cell migration via a Ga12/13 and Rho pathway.” Journal of Biological Chemistry 283.21 (2008): 14469-14478; and in Chen, Yang, et al. “GPR56 is essential for testis development and male fertility in mice.” Developmental Dynamics 239.12 (2010): 3358-3367.

Antibodies targeting CD71 are known in the art, including, for example, the anti-CD71 antibody anti-Tfr1 H68.4 described in Byrne, et al. “Ferristatin II promotes degradation of transferrin receptor-1 in vitro and in vivo.” PLoS One 8.7 (2013): e70199. Additional anti-CD71 antibodies include, for example, those described in Hanamachi, et al. “Novel method for screening functional antibody with comprehensive analysis of its immunoliposome.” Scientific reports 11.1 (2021): 1-13; and in Kono, et al. “Morphological definition of CD71 positive reticulocytes by various staining techniques and electron microscopy compared to reticulocytes detected by an automated hematology analyzer.” Clinica Chimica Acta 404.2 (2009): 105-110.

The antibodies described in the foregoing are merely exemplary and are not meant to limit in any way the scope of the present disclosure. Additional binding agents, including antibodies, suitable for incorporation into the methods and bispecific binding agents of the present disclosure will be evident to one of ordinary skill.

Although aspects of the present disclosure have been described with reference to the disclosed embodiments, one skilled in the art will readily appreciate that the specific examples disclosed are only illustrative of these aspects and in no way limit the present disclosure. Various modifications can be made without departing from the spirit of the present disclosure.

In some embodiments, the first binding domain comprises a heavy chain (HC) sequence, a variable heavy (VH) sequence, a light chain (LC) sequence, and a variable light (VL) sequence. In some embodiments, the first binding domain comprises an HC sequence and a VH sequence. The first binding domain comprising an HC sequence, a VH sequence, an LC sequence, and a VL sequence may comprises one or more sequences listed in Table 1 or 2. The first binding domain comprising an HC sequence, a VH sequence, an LC sequence, and a VL sequence may comprise at least 70% sequence identity to one or more sequences listed in Table 1 or 2. In some cases, the first binding domain comprising an HC sequence, a VH sequence, an LC sequence, and a VL sequence comprises at least 75% sequence identity to one or more sequences listed in Table 1 or 2. In some cases, the first binding domain comprising an HC sequence, a VH sequence, an LC sequence, and a VL sequence comprises at least 80% sequence identity to one or more sequences listed in Table 1 or 2. In some cases, the first binding domain comprising an HC sequence, a VH sequence, an LC sequence, and a VL sequence comprises at least 85% sequence identity to one or more sequences listed in Table 1 or 2. In some cases, the first binding domain comprising an HC sequence, a VH sequence, an LC sequence, and a VL sequence comprises at least 90% sequence identity to one or more sequences listed in Table 1 or 2. In some cases, the first binding domain comprising an HC sequence, a VH sequence, an LC sequence, and a VL sequence comprises at least 91% sequence identity to one or more sequences listed in Table 1 or 2. In some cases, the first binding domain comprising an HC sequence, a VH sequence, an LC sequence, and a VL sequence comprises at least 92% sequence identity to one or more sequences listed in Table 1 or 2. In some cases, the first binding domain comprising an HC sequence, a VH sequence, an LC sequence, and a VL sequence comprises at least 93% sequence identity to one or more sequences listed in Table 1 or 2. In some cases, the first binding domain comprising an HC sequence, a VH sequence, an LC sequence, and a VL sequence comprises at least 94% sequence identity to one or more sequences listed in Table 1 or 2. In some cases, the first binding domain comprising an HC sequence, a VH sequence, an LC sequence, and a VL sequence comprises at least 95% sequence identity to one or more sequences listed in Table 1 or 2. In some cases, the first binding domain comprising an HC sequence, a VH sequence, an LC sequence, and a VL sequence comprises at least 96% sequence identity to one or more sequences listed in Table 1 or 2. In some cases, the first binding domain comprising an HC sequence, a VH sequence, an LC sequence, and a VL sequence comprises at least 97% sequence identity to one or more sequences listed in Table 1 or 2. In some cases, the first binding domain comprising an HC sequence, a VH sequence, an LC sequence, and a VL sequence comprises at least 98% sequence identity to one or more sequences listed in Table 1 or 2. In some cases, the first binding domain comprising an HC sequence, a VH sequence, an LC sequence, and a VL sequence comprises at least 99% sequence identity to one or more sequences listed in Table 1 or 2. In some cases, the first binding domain comprising an HC sequence, a VH sequence, an LC sequence, and a VL sequence comprises at least 99.5% sequence identity to one or more sequences listed in Table 1 or 2. In some cases, the first binding domain comprising an HC sequence, a VH sequence, an LC sequence, and a VL sequence comprises at least 99.9% sequence identity to one or more sequences listed in Table 1 or 2.

In some embodiments, the first binding domain comprises an antibody comprising a heavy chain (HC) sequence, a variable heavy (VH) sequence, a light chain (LC) sequence, and a variable light (VL) sequence. In some embodiments, the first binding domain comprises an antibody comprising an HC sequence and a VH sequence. The first binding domain comprising an antibody comprising an HC sequence, a VH sequence, an LC sequence, and a VL sequence may comprise one or more sequences listed in Table 1 or 2. The first binding domain comprising an antibody comprising an HC sequence, a VH sequence, an LC sequence, and a VL sequence may comprise at least 70% sequence identity to one or more sequences listed in Table 1 or 2. In some cases, the first binding domain comprising an antibody comprising an HC sequence, a VH sequence, an LC sequence, and a VL sequence comprises at least 75% sequence identity to one or more sequences listed in Table 1 or 2. In some cases, the first binding domain comprising an antibody comprising an HC sequence, a VH sequence, an LC sequence, and a VL sequence comprises at least 80% sequence identity to one or more sequences listed in Table 1 or 2. In some cases, the first binding domain comprising an antibody comprising an HC sequence, a VH sequence, an LC sequence, and a VL sequence comprises at least 85% sequence identity to one or more sequences listed in Table 1 or 2. In some cases, the first binding domain comprising an antibody comprising an HC sequence, a VH sequence, an LC sequence, and a VL sequence comprises at least 90% sequence identity to one or more sequences listed in Table 1 or 2. In some cases, the first binding domain comprising an antibody comprising an HC sequence, a VH sequence, an LC sequence, and a VL sequence comprises at least 91% sequence identity to one or more sequences listed in Table 1 or 2. In some cases, the first binding domain comprising an antibody comprising an HC sequence, a VH sequence, an LC sequence, and a VL sequence comprises at least 92% sequence identity to one or more sequences listed in Table 1 or 2. In some cases, the first binding domain comprising an antibody comprising an HC sequence, a VH sequence, an LC sequence, and a VL sequence comprises at least 93% sequence identity to one or more sequences listed in Table 1 or 2. In some cases, the first binding domain comprising an antibody comprising an HC sequence, a VH sequence, an LC sequence, and a VL sequence comprises at least 94% sequence identity to one or more sequences listed in Table 1 or 2. In some cases, the first binding domain comprising an antibody comprising an HC sequence, a VH sequence, an LC sequence, and a VL sequence comprises at least 95% sequence identity to one or more sequences listed in Table 1 or 2. In some cases, the first binding domain comprising an antibody comprising an HC sequence, a VH sequence, an LC sequence, and a VL sequence comprises at least 96% sequence identity to one or more sequences listed in Table 1 or 2. In some cases, the first binding domain comprising an antibody comprising an HC sequence, a VH sequence, an LC sequence, and a VL sequence comprises at least 97% sequence identity to one or more sequences listed in Table 1 or 2. In some cases, the first binding domain comprising an antibody comprising an HC sequence, a VH sequence, an LC sequence, and a VL sequence comprises at least 98% sequence identity to one or more sequences listed in Table 1 or 2. In some cases, the first binding domain comprising an antibody comprising an HC sequence, a VH sequence, an LC sequence, and a VL sequence comprises at least 99% sequence identity to one or more sequences listed in Table 1 or 2. In some cases, the first binding domain comprising an antibody comprising an HC sequence, a VH sequence, an LC sequence, and a VL sequence comprises at least 99.5% sequence identity to one or more sequences listed in Table 1 or 2. In some cases, the first binding domain comprising an antibody comprising an HC sequence, a VH sequence, an LC sequence, and a VL sequence comprises at least 99.9% sequence identity to one or more sequences listed in Table 1 or 2.

In some embodiments, the antibodies targeting the internalizing receptor protein comprise sequences listed Table 1. In some embodiments, the antibodies targeting the internalizing receptor protein comprise at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, at least 99.9%, or at least 99.9% sequence identity to the sequences listed Table 1.

In some cases, the antibodies targeting the internalizing receptor protein may bind the same epitope as any one of the antibodies listed in Table 1. The antibodies targeting the internalizing receptor protein may bind to an epitope that comprises about 70% sequence identity to the epitope to which any one of the antibodies listed in Table 1 binds. The antibodies targeting the internalizing receptor protein may bind to an epitope that comprises about 75% sequence identity to the epitope to which any one of the antibodies listed in Table 1 binds. The antibodies targeting the internalizing receptor protein may bind to an epitope that comprises about 80% sequence identity to the epitope to which any one of the antibodies listed in Table 1 binds. The antibodies targeting the internalizing receptor protein may bind to an epitope that comprises about 85% sequence identity to the epitope to which any one of the antibodies listed in Table 1 binds. The antibodies targeting the internalizing receptor protein may bind to an epitope that comprises about 90% sequence identity to the epitope to which any one of the antibodies listed in Table 1 binds. The antibodies targeting the internalizing receptor protein may bind to an epitope that comprises about 95% sequence identity to the epitope to which any one of the antibodies listed in Table 1 binds. The antibodies targeting the internalizing receptor protein may bind to an epitope that comprises about 99% sequence identity to the epitope to which any one of the antibodies listed in Table 1 binds.

In some embodiments, the antibodies targeting the internalizing receptor protein may bind with a similar affinity as any one of the antibodies listed in Table 1 (Table 5 lists affinities of certain monovalent binders). Table 5 describes monovalent Kds to particular cell surface monovalent proteins. In certain embodiments, multispecific binding agents have a Kd less than, more than, within 10%, within 20%, within 30%, within 40%, within 50%, withing 75%, or within 100% of the binding affinity of the monovalent binding agent. For example, in Table 5, the monovalent binding affinities are described for certain CD71 monovalent binders. When those CD71 binding arms are incorporated in the monovalent binding agent of the disclosure, the binding affinity of the multispecific binding agent may be within an order of magnitude or an order of two-fold as the binding affinity of the monovalent binding agent. For example, the binding affinity of the monovalent binding agent has a Kd of between 0.1 nM and 100 nM. When incorporated into the multispecific binding agent, the Kd may be within the same range. Alternatively, the binding affinity may be slightly greater than, but within two fold of the monovalent binding affinity. The binding affinity may be within three fold of the monovalent binding affinity.

The antibodies targeting the internalizing receptor protein may bind to an epitope that comprises about 70% sequence identity to the epitope to which any one of the antibodies listed in Table 1 binds with a similar affinity as any one of the antibodies listed in Table 1. The antibodies targeting the internalizing receptor protein may bind to an epitope that comprises about 75% sequence identity to the epitope to which any one of the antibodies listed in Table 1 binds with a similar affinity as any one of the antibodies listed in Table 1. The antibodies targeting the internalizing receptor protein may bind to an epitope that comprises about 80% sequence identity to the epitope to which any one of the antibodies listed in Table 1 binds with a similar affinity as any one of the antibodies listed in Table 1. The antibodies targeting the internalizing receptor protein may bind to an epitope that comprises about 85% sequence identity to the epitope to which any one of the antibodies listed in Table 1 binds with a similar affinity as any one of the antibodies listed in Table 1. The antibodies targeting the internalizing receptor protein may bind to an epitope that comprises about 90% sequence identity to the epitope to which any one of the antibodies listed in Table 1 binds with a similar affinity as any one of the antibodies listed in Table 1. The antibodies targeting the internalizing receptor protein may bind to an epitope that comprises about 95% sequence identity to the epitope to which any one of the antibodies listed in Table 1 binds with a similar affinity as any one of the antibodies listed in Table 1. The antibodies targeting the internalizing receptor protein may bind to an epitope that comprises about 99% sequence identity to the epitope to which any one of the antibodies listed in Table 1 binds with a similar affinity as any one of the antibodies listed in Table 1.

In some embodiments, the antibodies targeting the internalizing receptor protein may bind the same epitope as any one of the antibodies listed in Table 1 binds with a different affinity as compared to any one of the antibodies listed in Table 1. The antibodies targeting the internalizing receptor protein may bind to an epitope that comprises about 70% sequence identity to the epitope to which any one of the antibodies listed in Table 1 binds with a different affinity as compared to any one of the antibodies listed in Table 1. The antibodies targeting the internalizing receptor protein may bind to an epitope that comprises about 75% sequence identity to the epitope to which any one of the antibodies listed in Table 1 binds with a different affinity as compared to any one of the antibodies listed in Table 1. The antibodies targeting the internalizing receptor protein may bind to an epitope that comprises about 80% sequence identity to the epitope to which any one of the antibodies listed in Table 1 binds with a different affinity as compared to any one of the antibodies listed in Table 1. The antibodies targeting the internalizing receptor protein may bind to an epitope that comprises about 85% sequence identity to the epitope to which any one of the antibodies listed in Table 1 binds with a different affinity as compared to any one of the antibodies listed in Table 1. The antibodies targeting the internalizing receptor protein may bind to an epitope that comprises about 90% sequence identity to the epitope to which any one of the antibodies listed in Table 1 binds with a different affinity as compared to any one of the antibodies listed in Table 1. The antibodies targeting the internalizing receptor protein may bind to an epitope that comprises about 95% sequence identity to the epitope to which any one of the antibodies listed in Table 1 binds with a different affinity as compared to any one of the antibodies listed in Table 1. The antibodies targeting the internalizing receptor protein may bind to an epitope that comprises about 99% sequence identity to the epitope to which any one of the antibodies listed in Table 1 binds with a different affinity as compared to any one of the antibodies listed in Table 1.

The antibodies targeting the internalizing receptor protein may bind to an epitope that comprises a different epitope than the epitope to which any one of the antibodies listed in Table 1 binds. The antibodies targeting the internalizing receptor protein may bind to an epitope that comprises a different epitope than the epitope to which any one of the antibodies listed in Table 1 binds, wherein the epitopes do not bind to any of the same amino acids on the internalizing receptor protein. The antibodies targeting the internalizing receptor protein may bind to an epitope that comprises a different epitope than the epitope to which any one of the antibodies listed in Table 1 binds, wherein the epitopes bind to any one or more of the same amino acids on the internalizing receptor protein. The antibodies targeting the internalizing receptor protein may bind to an epitope that comprises a different epitope than the epitope to which any one of the antibodies listed in Table 1 binds, wherein the epitopes bind to any two or more of the same amino acids on the internalizing receptor protein. The antibodies targeting the internalizing receptor protein may bind to an epitope that comprises a different epitope than the epitope to which any one of the antibodies listed in Table 1 binds, wherein the epitopes bind to any three or more of the same amino acids on the internalizing receptor protein. The antibodies targeting the internalizing receptor protein may bind to an epitope that comprises a different epitope than the epitope to which any one of the antibodies listed in Table 1 binds, wherein the epitopes bind to any four or more of the same amino acids on the internalizing receptor protein. The antibodies targeting the internalizing receptor protein may bind to an epitope that comprises a different epitope than the epitope to which any one of the antibodies listed in Table 1 binds, wherein the epitopes bind to any five or more of the same amino acids on the internalizing receptor protein. The antibodies targeting the internalizing receptor protein may bind to an epitope that comprises a different epitope than the epitope to which any one of the antibodies listed in Table 1 binds, wherein the epitopes bind to any six or more of the same amino acids on the internalizing receptor protein. The antibodies targeting the internalizing receptor protein may bind to an epitope that comprises a different epitope than the epitope to which any one of the antibodies listed in Table 1 binds, wherein the epitopes bind to any seven or more of the same amino acids on the internalizing receptor protein. The antibodies targeting the internalizing receptor protein may bind to an epitope that comprises a different epitope than the epitope to which any one of the antibodies listed in Table 1 binds, wherein the epitopes bind to any eight or more of the same amino acids on the internalizing receptor protein. The antibodies targeting the internalizing receptor protein may bind to an epitope that comprises a different epitope than the epitope to which any one of the antibodies listed in Table 1 binds, wherein the epitopes bind to any nine or more of the same amino acids on the internalizing receptor protein. The antibodies targeting the internalizing receptor protein may bind to an epitope that comprises a different epitope than the epitope to which any one of the antibodies listed in Table 1 binds, wherein the epitopes bind to any ten or more of the same amino acids on the internalizing receptor protein.

In some embodiments, the antibodies targeting the degrader protein comprise sequences listed Table 1. In some embodiments, the antibodies targeting the degrader protein comprise at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, at least 99.9%, or at least 99.9% sequence identity to the sequences listed Table 1.

In some cases, the antibodies targeting the degrader protein may bind the same epitope as any one of the antibodies listed in Table 1. The antibodies targeting the degrader protein may bind to an epitope that comprises about 70% sequence identity to the epitope to which any one of the antibodies listed in Table 1 binds. The antibodies targeting the degrader protein may bind to an epitope that comprises about 75% sequence identity to the epitope to which any one of the antibodies listed in Table 1 binds. The antibodies targeting the degrader protein may bind to an epitope that comprises about 80% sequence identity to the epitope to which any one of the antibodies listed in Table 1 binds. The antibodies targeting the degrader protein may bind to an epitope that comprises about 85% sequence identity to the epitope to which any one of the antibodies listed in Table 1 binds. The antibodies targeting the degrader protein may bind to an epitope that comprises about 90% sequence identity to the epitope to which any one of the antibodies listed in Table 1 binds. The antibodies targeting the degrader protein may bind to an epitope that comprises about 95% sequence identity to the epitope to which any one of the antibodies listed in Table 1 binds. The antibodies targeting the degrader protein may bind to an epitope that comprises about 99% sequence identity to the epitope to which any one of the antibodies listed in Table 1 binds.

The antibodies targeting the degrader protein may bind to an epitope that comprises a different epitope than the epitope to which any one of the antibodies listed in Table 1 binds. The antibodies targeting the degrader protein may bind to an epitope that comprises a different epitope than the epitope to which any one of the antibodies listed in Table 1 binds, wherein the epitopes do not bind to any of the same amino acids on the degrader protein. The antibodies targeting the degrader protein may bind to an epitope that comprises a different epitope than the epitope to which any one of the antibodies listed in Table 1 binds, wherein the epitopes bind to any one or more of the same amino acids on the degrader protein. The antibodies targeting the degrader protein may bind to an epitope that comprises a different epitope than the epitope to which any one of the antibodies listed in Table 1 binds, wherein the epitopes bind to any two or more of the same amino acids on the degrader protein. The antibodies targeting the degrader protein may bind to an epitope that comprises a different epitope than the epitope to which any one of the antibodies listed in Table 1 binds, wherein the epitopes bind to any three or more of the same amino acids on the degrader protein. The antibodies targeting the degrader protein may bind to an epitope that comprises a different epitope than the epitope to which any one of the antibodies listed in Table 1 binds, wherein the epitopes bind to any four or more of the same amino acids on the degrader protein. The antibodies targeting the degrader protein may bind to an epitope that comprises a different epitope than the epitope to which any one of the antibodies listed in Table 1 binds, wherein the epitopes bind to any five or more of the same amino acids on the degrader protein. The antibodies targeting the degrader protein may bind to an epitope that comprises a different epitope than the epitope to which any one of the antibodies listed in Table 1 binds, wherein the epitopes bind to any six or more of the same amino acids on the degrader protein. The antibodies targeting the degrader protein may bind to an epitope that comprises a different epitope than the epitope to which any one of the antibodies listed in Table 1 binds, wherein the epitopes bind to any seven or more of the same amino acids on the degrader protein. The antibodies targeting the degrader protein may bind to an epitope that comprises a different epitope than the epitope to which any one of the antibodies listed in Table 1 binds, wherein the epitopes bind to any eight or more of the same amino acids on the degrader protein. The antibodies targeting the degrader protein may bind to an epitope that comprises a different epitope than the epitope to which any one of the antibodies listed in Table 1 binds, wherein the epitopes bind to any nine or more of the same amino acids on the degrader protein. The antibodies targeting the degrader protein may bind to an epitope that comprises a different epitope than the epitope to which any one of the antibodies listed in Table 1 binds, wherein the epitopes bind to any ten or more of the same amino acids on the degrader protein.

TABLE 1
Exemplary antibody sequences targeting the internalizing receptor protein or
degrader protein.

| | | SEQ | | SEQ | | SEQ | | SEQ | |
|---|---|---|---|---|---|---|---|---|---|
| | Arm 1 | ID | | ID | VH | ID | | ID | VL |
| ID | Target | NO | HC sequence | NO | sequence | NO | LC sequence | NO | sequence |
| EPI818 | MET | 1 | QVQLVQSGAEVKKPGAS | 2 | QVQLVQSGA | 3 | DIQMTQSPSSV | 4 | DIQMTQS |
| | | | VKVSCETSGYTFTSYGISW | | EVKKPGASV | | SASVGDRVTITC | | PSSVSASV |
| | | | VRQAPGHGLEWMGWISA | | KVSCETSGYT | | RASQGISNWLA | | GDRVTITC |
| | | | YNGYTNYAQKLQGRVTM | | FTSYGISWVR | | WFQHKPGKAPK | | RASQGISN |
| | | | TTDTSTSTAYMELRSLRSD | | QAPGHGLEW | | LLIYAASSLLSG | | WLAWFQ |
| | | | DTAVYYCARDLRGTNYFD | | MGWISAYNG | | VPSRFSGSGSGT | | HKPGKAP |
| | | | YWGQGTLVTVSSASTKGP | | YTNYAQKLQ | | DFTLTISSLQPED | | KLLIYAAS |
| | | | SVFPLAPSSKSTSGGTAAL | | GRVTMTTDTS | | FATYYCQQANS | | SLLSGVPS |
| | | | GCLVKDYFPEPVTVSWNS | | TSTAYMELRS | | FPITFGQGTRLEI | | RFSGSGSG |
| | | | GALTSGVHTFPAVLQSSGL | | LRSDDTAVY | | KRTVAAPSVFIF | | TDFTLTIS |
| | | | YSLSSVVTVPSSSLGTQTYI | | YCARDLRGT | | PPSDEQLKSGTA | | SLQPEDFA |
| | | | CNVNHKPSNTKVDKKVEP | | NYFDYWGQG | | SVVCLLNNFYPR | | TYYCQQA |
| | | | KSCDKTHTCPPCPAPELLG | | TLVTVSS | | EAKVQWKVDN | | NSFPITFG |
| | | | GPSVFLFPPKPKDTLMISR | | | | ALQSGNSQESVT | | QGTRLEIK |
| | | | TPEVTCVVVDVSHEDPEV | | | | EQDSKDSTYSLS | | |
| | | | KFNWYVDGVEVHNAKTK | | | | STLTLSKADYEK | | |
| | | | PREEQYNSTYRVVSVLTV | | | | HKVYACEVTHQ | | |
| | | | LHQDWLNGKEYKCKVSN | | | | GLSSPVTKSFNR | | |
| | | | KALPAPIEKTISKAKGQPR | | | | GEC | | |
| | | | EPQVYTLPPSRDELTKNQV | | | | | | |
| | | | SLWCLVKGFYPSDIAVEW | | | | | | |
| | | | ESNGQPENNYKTTPPVLDS | | | | | | |
| | | | DGSFFLYSKLTVDKSRWQ | | | | | | |
| | | | QGNVFSCSVMHEALHNH | | | | | | |
| | | | YTQKSLSLSPGK | | | | | | |
| EPI1097 | LGR5 | 5 | EVQLVQSGSKLKKPGASV | 6 | EVQLVQSGSK | 7 | DIQMTQSPSSL | 8 | DIQMTQS |
| | | | KVSCKASGYTFTSYTMNW | | LKKPGASVK | | SASVGDRVTITC | | PSSLSASV |
| | | | VRQAPGQGLEWMGWINT | | VSCKASGYTF | | RASQSISSYLNW | | GDRVTITC |
| | | | DTGDPTYAQGFTGRFVFS | | TSYTMNWVR | | YQQKPGKAPKL | | RASQSISS |
| | | | LDTSVSTAFLQINSLKAED | | QAPGQGLEW | | LIYAASSLQSGV | | YLNWYQ |
| | | | TAVYYCARGDCDSTSCYR | | MGWINTDTG | | PSRFSGSGSGTD | | QKPGKAP |
| | | | YSYGYEDYWGQGTLVTV | | DPTYAQGFTG | | FTLTISSLQPEDF | | KLLIYAAS |
| | | | SSASTKGPSVFPLAPSSKST | | RFVFSLDTSV | | ATYYCQQSYST | | SLQSGVPS |
| | | | SGGTAALGCLVKDYFPEP | | STAFLQINSLK | | PPTFGQGTKVEI | | RFSGSGSG |
| | | | VTVSWNSGALTSGVHTFP | | AEDTAVYYC | | KRTVAAPSVFIF | | TDFTLTIS |
| | | | AVLQSSGLYSLSSVVTVPS | | ARGDCDSTSC | | PPSDEQLKSGTA | | SLQPEDFA |
| | | | SSLGTQTYICNVNHKPSNT | | YRYSYGYED | | SVVCLLNNFYPR | | TYYCQQS |
| | | | KVDKKVEPKSCDKTHTCP | | YWGQGTLVT | | EAKVQWKVDN | | YSTPPTFG |
| | | | PCPAPELLGGPSVFLFPPKP | | VSS | | ALQSGNSQESVT | | QGTKVEI |
| | | | KDTLMISRTPEVTCVVVD | | | | EQDSKDSTYSLS | | K |
| | | | VSHEDPEVKFNWYVDGV | | | | STLTLSKADYEK | | |
| | | | EVHNAKTKPREEQYNSTY | | | | HKVYACEVTHQ | | |
| | | | RVVSVLTVLHQDWLNGK | | | | GLSSPVTKSFNR | | |
| | | | EYKCKVSNKALPAPIEKTI | | | | GEC | | |
| | | | SKAKGQPREPQVYTLPPSR | | | | | | |
| | | | DELTKNQVSLWCLVKGFY | | | | | | |
| | | | PSDIAVEWESNGQPENNY | | | | | | |
| | | | KTTPPVLDSDGSFFLYSKL | | | | | | |
| | | | TVDKSRWQQGNVFSCSV | | | | | | |
| | | | MHEALHNHYTQKSLSLSP | | | | | | |
| | | | GK | | | | | | |
| EPI733 | RSV F | 9 | QVTLRESGPALVKPTQTL | 10 | QVTLRESGPA | 11 | DIQMTQSPSTL | 12 | DIQMTQS |
| | Pro- | | TLTCTFSGFSLSTSGMSVG | | LVKPTQTLTL | | SASVGDRVTITC | | PSTLSASV |
| | tein | | WIRQPPGKALEWLADIW | | TCTFSGFSLST | | KCQLSVGYMH | | GDRVTITC |
| | | | WDDKKDYNPSLKSRLTIS | | SGMSVGWIR | | WYQQKPGKAPK | | KCQLSVG |
| | | | KDTSKNQVVLKVTNMDP | | QPPGKALEW | | LLIYDTSKLASG | | YMHWYQ |
| | | | ADTATYYCARSMITNWYF | | LADIWWDDK | | VPSRFSGSGSGT | | QKPGKAP |
| | | | DVWGAGTTVTVSSASTKG | | KDYNPSLKSR | | EFTLTISSLQPDD | | KLLIYDTS |
| | | | PSVFPLAPSSKSTSGGTAA | | LTISKDTSKN | | FATYYCFQGSG | | KLASGVP |
| | | | LGCLVKDYFPEPVTVSWN | | QVVLKVTNM | | YPFTFGGGTKLE | | SRFSGSGS |
| | | | SGALTSGVHTFPAVLQSSG | | DPADTATYY | | IKRTVAAPSVFIF | | GTEFTLTI |
| | | | LYSLSSVVTVPSSSLGTQT | | CARSMITNW | | PPSDEQLKSGTA | | SSLQPDDF |
| | | | YICNVNHKPSNTKVDKKV | | YFDVWGAGT | | SVVCLLNNFYPR | | ATYYCFQ |
| | | | EPKSCDKTHTCPPCPAPEL | | TVTVSS | | EAKVQWKVDN | | GSGYPFTF |
| | | | LGGPSVFLFPPKPKDTLMI | | | | ALQSGNSQESVT | | GGGTKLEI |
| | | | SRTPEVTCVVVDVSHEDP | | | | EQDSKDSTYSLS | | K |
| | | | EVKFNWYVDGVEVHNAK | | | | STLTLSKADYEK | | |
| | | | TKPREEQYNSTYRVVSVL | | | | HKVYACEVTHQ | | |
| | | | TVLHQDWLNGKEYKCKV | | | | GLSSPVTKSFNR | | |
| | | | SNKALPAPIEKTISKAKGQ | | | | GEC | | |
| | | | PREPQVYTLPPSRDELTKN | | | | | | |
| | | | QVSLWCLVKGFYPSDIAV | | | | | | |
| | | | EWESNGQPENNYKTTPPV | | | | | | |
| | | | LDSDGSFFLYSKLTVDKSR | | | | | | |
| | | | WQQGNVFSCSVMHEALH | | | | | | |
| | | | NHYTQKSLSLSPGKGGSH | | | | | | |
| | | | HHHHH | | | | | | |
| EPI798 | None | 13 | DKTHTCPPCPAPELLGGPS | | | | | | |
| | | | VFLFPPKPKDTLMISRTPE | | | | | | |
| | | | VTCVVVDVSHEDPEVKFN | | | | | | |
| | | | WYVDGVEVHNAKTKPRE | | | | | | |
| | | | EQYNSTYRVVSVLTVLHQ | | | | | | |
| | | | DWLNGKEYKCKVSNKAL | | | | | | |
| | | | PAPIEKTISKAKGQPREPQ | | | | | | |
| | | | VYTLPPSRDELTKNQVSL | | | | | | |
| | | | WCLVKGFYPSDIAVEWES | | | | | | |
| | | | NGQPENNYKTTPPVLDSD | | | | | | |
| | | | GSFFLYSKLTVDKSRWQQ | | | | | | |
| | | | GNVFSCSVMHEALHNHYT | | | | | | |
| | | | QKSLSLSPGKGGSHHHHH | | | | | | |
| | | | H | | | | | | |
| EPI808 | TROP2 | 14 | QVQLQQSGSELKKPGASV | 15 | QVQLQQSGSE | 16 | DIQLTQSPSSLS | 17 | DIQLTQSP |
| | | | KVSCKASGYTFTNYGMN | | LKKPGASVK | | ASVGDRVSITCK | | SSLSASVG |
| | | | WVKQAPGQGLKWMGWI | | VSCKASGYTF | | ASQDVSIAVAW | | DRVSITCK |
| | | | NTYTGEPTYTDDFKGRFA | | TNYGMNWV | | YQQKPGKAPKL | | ASQDVSIA |
| | | | FSLDTSVSTAYLQISSLKA | | KQAPGQGLK | | LIYSASYRYTGV | | VAWYQQ |
| | | | DDTAVYFCARGGFGSSYW | | WMGWINTYT | | PDRFSGSGSGTD | | KPGKAPK |
| | | | YFDVWGQGSLVTVSSAST | | GEPTYTDDFK | | FTLTISSLQPEDF | | LLIYSASY |
| | | | KGPSVFPLAPSSKSTSGGT | | GRFAFSLDTS | | AVYYCQQHYIT | | RYTGVPD |
| | | | AALGCLVKDYFPEPVTVS | | VSTAYLQISSL | | PLTFGAGTKVEI | | RFSGSGSG |
| | | | WNSGALTSGVHTFPAVLQ | | KADDTAVYF | | KRTVAAPSVFIF | | TDFTLTIS |
| | | | SSGLYSLSSVVTVPSSSLG | | CARGGFGSSY | | PPSDEQLKSGTA | | SLQPEDFA |
| | | | TQTYICNVNHKPSNTKVD | | WYFDVWGQ | | SVVCLLNNFYPR | | VYYCQQH |
| | | | KKVEPKSCDKTHTCPPCP | | GSLVTVSS | | EAKVQWKVDN | | YITPLTFG |
| | | | APELLGGPSVFLFPPKPKD | | | | ALQSGNSQESVT | | AGTKVEI |
| | | | TLMISRTPEVTCVVVDVSH | | | | EQDSKDSTYSLS | | K |
| | | | EDPEVKFNWYVDGVEVH | | | | STLTLSKADYEK | | |
| | | | NAKTKPREEQYNSTYRVV | | | | HKVYACEVTHQ | | |
| | | | SVLTVLHQDWLNGKEYK | | | | GLSSPVTKSFNR | | |
| | | | CKVSNKALPAPIEKTISKA | | | | GEC | | |
| | | | KGQPREPQVYTLPPSRDEL | | | | | | |
| | | | TKNQVSLWCLVKGFYPSD | | | | | | |
| | | | LAVEWESNGQPENNYKTT | | | | | | |
| | | | PPVLDSDGSFFLYSKLTVD | | | | | | |
| | | | KSRWQQGNVFSCSVMHE | | | | | | |
| | | | ALHNHYTQKSLSLSPGK | | | | | | |
| EPI809 | RNF43 | 18 | QVQLQESGPGLVKPSETLS | 19 | QVQLQESGPG | 20 | DIQMTQSPSSL | 21 | DIQMTQS |
| | | | LTCTVSGGSISSSNYYWG | | LVKPSETLSL | | SASVGDRVTITC | | PSSLSASV |
| | | | WIRQPPGKGLEWIGNIYYR | | TCTVSGGSISS | | RASQSISSYLNW | | GDRVTITC |
| | | | GYTYYNPSLKSRVTISVDT | | SNYYWGWIR | | YQQKPGKAPKL | | RASQSISS |
| | | | SKKQFSLTLSSVTAADTA | | QPPGKGLEWI | | LIYAASSLQSGV | | YLNWYQ |
| | | | MYYCAREGSDYGDYVGA | | GNIYYRGYTY | | PSRFSGSGSGTD | | QKPGKAP |
| | | | FDIWDQGTMVTVSSASTK | | YNPSLKSRVT | | FTLTISSLQPEDF | | KLLIYAAS |
| | | | GPSVFPLAPSSKSTSGGTA | | ISVDTSKKQF | | ATYYCQQSYST | | SLQSGVPS |
| | | | ALGCLVKDYFPEPVTVSW | | SLTLSSVTAA | | PPTFGQGTKVEI | | RFSGSGSG |
| | | | NSGALTSGVHTFPAVLQSS | | DTAMYYCAR | | KRTVAAPSVFIF | | TDFTLTIS |
| | | | GLYSLSSVVTVPSSSLGTQ | | EGSDYGDYV | | PPSDEQLKSGTA | | SLQPEDFA |
| | | | TYICNVNHKPSNTKVDKK | | GAFDIWDQG | | SVVCLLNNFYPR | | TYYCQQS |
| | | | VEPKSCDKTHTCPPCPAPE | | TMVTVSS | | EAKVQWKVDN | | YSTPPTFG |
| | | | LLGGPSVFLFPPKPKDTLM | | | | ALQSGNSQESVT | | QGTKVEI |
| | | | ISRTPEVTCVVVDVSHEDP | | | | EQDSKDSTYSLS | | K |
| | | | EVKFNWYVDGVEVHNAK | | | | STLTLSKADYEK | | |
| | | | TKPREEQYNSTYRVVSVL | | | | HKVYACEVTHQ | | |
| | | | TVLHQDWLNGKEYKCKV | | | | GLSSPVTKSFNR | | |
| | | | SNKALPAPIEKTISKAKGQ | | | | GEC | | |
| | | | PREPQVYTLPPSRDELTKN | | | | | | |
| | | | QVSLWCLVKGFYPSDIAV | | | | | | |
| | | | EWESNGQPENNYKTTPPV | | | | | | |
| | | | LDSDGSFFLYSKLTVDKSR | | | | | | |
| | | | WQQGNVFSCSVMHEALH | | | | | | |
| | | | NHYTQKSLSLSPGK | | | | | | |
| EPI810 | RNF43 | 22 | EVQLVQSGAEVKKPGASV | 23 | EVQLVQSGAE | 24 | EIVMTQSPATL | 25 | EIVMTQSP |
| | | | KVSCKASGYTFTTYTIHW | | VKKPGASVK | | SVSPGERATLSC | | ATLSVSPG |
| | | | VRQAPGQGLEWMGYINPR | | VSCKASGYTF | | KASQNVGINVA | | ERATLSC |
| | | | SGYTEYNQKFQDRVTMTR | | TTYTIHWVRQ | | WYQQKPGQAPR | | KASQNVG |
| | | | DTSTSTVYMELSSLRSEDT | | APGQGLEWM | | ALIYSASYRYSG | | INVAWYQ |
| | | | AVYYCARSYEFWGQGTT | | GYINPRSGYT | | IPARFSGSGSGT | | QKPGQAP |
| | | | VTVSSASTKGPSVFPLAPS | | EYNQKFQDR | | EFTLTISSLQSED | | RALIYSAS |
| | | | SKSTSGGTAALGCLVKDY | | VTMTRDTSTS | | FAVYYCHQYKT | | YRYSGIPA |
| | | | FPEPVTVSWNSGALTSGV | | TVYMELSSLR | | YPYTFGGGTKL | | RFSGSGSG |
| | | | HTFPAVLQSSGLYSLSSVV | | SEDTAVYYC | | EIKRTVAAPSVFI | | TEFTLTIS |
| | | | TVPSSSLGTQTYICNVNHK | | ARSYEFWGQ | | FPPSDEQLKSGT | | SLQSEDFA |
| | | | PSNTKVDKKVEPKSCDKT | | GTTVTVSS | | ASVVCLLNNFY | | VYYCHQY |
| | | | HTCPPCPAPELLGGPSVFL | | | | PREAKVQWKVD | | KTYPYTF |
| | | | FPPKPKDTLMISRTPEVTC | | | | NALQSGNSQES | | GGGTKLEI |
| | | | VVVDVSHEDPEVKFNWY | | | | VTEQDSKDSTYS | | K |
| | | | VDGVEVHNAKTKPREEQY | | | | LSSTLTLSKADY | | |
| | | | NSTYRVVSVLTVLHQDWL | | | | EKHKVYACEVT | | |
| | | | NGKEYKCKVSNKALPAPI | | | | HQGLSSPVTKSF | | |
| | | | EKTISKAKGQPREPQVYTL | | | | NRGEC | | |
| | | | PPSRDELTKNQVSLWCLV | | | | | | |
| | | | KGFYPSDIAVEWESNGQP | | | | | | |
| | | | ENNYKTTPPVLDSDGSFFL | | | | | | |
| | | | YSKLTVDKSRWQQGNVFS | | | | | | |
| | | | CSVMHEALHNHYTQKSLS | | | | | | |
| | | | LSPGK | | | | | | |
| EPI811 | RNF43 | 26 | AVQLVESGGGSVQPGRSM | 27 | AVQLVESGG | 28 | DVVLTQTPVSL | 29 | DVVLTQT |
| | | | RLSCAASGFTFSNYDMTW | | GSVQPGRSM | | SVTVGDQASISC | | PVSLSVTV |
| | | | VRQAPTKGLEWVASITSD | | RLSCAASGFT | | RSSQSLEYSDGY | | GDQASISC |
| | | | GGSTYSRDSVKGRFTISRD | | FSNYDMTWV | | SYLEWYLQKPG | | RSSQSLEY |
| | | | NAKSTLYLQMDSLRSEDT | | RQAPTKGLE | | QSPQLLIYEVSS | | SDGYSYL |
| | | | ATYYCTTDRGRYLPYYFD | | WVASITSDGG | | RFSGVPDRFIGS | | EWYLQKP |
| | | | YWGQGVMVTVSSASTKG | | STYSRDSVKG | | GSGTDFTLKISR | | GQSPQLLI |
| | | | PSVFPLAPSSKSTSGGTAA | | RFTISRDNAK | | VEPEDLGVYYC | | YEVSSRFS |
| | | | LGCLVKDYFPEPVTVSWN | | STLYLQMDSL | | FQAIHDPTFGAG | | GVPDRFIG |
| | | | SGALTSGVHTFPAVLQSSG | | RSEDTATYYC | | TKLELKRTVAA | | SGSGTDFT |
| | | | LYSLSSVVTVPSSSLGTQT | | TTDRGRYLPY | | PSVFIFPPSDEQL | | LKISRVEP |
| | | | YICNVNHKPSNTKVDKKV | | YFDYWGQGV | | KSGTASVVCLL | | EDLGVYY |
| | | | EPKSCDKTHTCPPCPAPEL | | MVTVSS | | NNFYPREAKVQ | | CFQAIHDP |
| | | | LGGPSVFLFPPKPKDTLMI | | | | WKVDNALQSG | | TFGAGTK |
| | | | SRTPEVTCVVVDVSHEDP | | | | NSQESVTEQDSK | | LELK |
| | | | EVKFNWYVDGVEVHNAK | | | | DSTYSLSSTLTL | | |
| | | | TKPREEQYNSTYRVVSVL | | | | SKADYEKHKVY | | |
| | | | TVLHQDWLNGKEYKCKV | | | | ACEVTHQGLSSP | | |
| | | | SNKALPAPIEKTISKAKGQ | | | | VTKSFNRGEC | | |
| | | | PREPQVYTLPPSRDELTKN | | | | | | |
| | | | QVSLWCLVKGFYPSDIAV | | | | | | |
| | | | EWESNGQPENNYKTTPPV | | | | | | |
| | | | LDSDGSFFLYSKLTVDKSR | | | | | | |
| | | | WQQGNVFSCSVMHEALH | | | | | | |
| | | | NHYTQKSLSLSPGK | | | | | | |
| EPI812 | HER3 | 30 | EVQLLESGGGLVQPGGSL | 31 | EVQLLESGGG | 32 | DIQMTQSPSSL | 33 | DIQMTQS |
| | | | RLSCAASGFTFSSYAMSW | | LVQPGGSLRL | | SASVGDRVTITC | | PSSLSASV |
| | | | VRQAPGKGLEWVSAINSQ | | SCAASGFTFS | | RASQGISNWLA | | GDRVTITC |
| | | | GKSTYYADSVKGRFTISRD | | SYAMSWVRQ | | WYQQKPGKAPK | | RASQGISN |
| | | | NSKNTLYLQMNSLRAEDT | | APGKGLEWV | | LLIYGASSLQSG | | WLAWYQ |
| | | | AVYYCARWGDEGFDIWG | | SAINSQGKST | | VPSRFSGSGSGT | | QKPGKAP |
| | | | QGTLVTVSSASTKGPSVFP | | YYADSVKGR | | DFTLTISSLQPED | | KLLIYGAS |
| | | | LAPSSKSTSGGTAALGCLV | | FTISRDNSKN | | FATYYCQQYSSF | | SLQSGVPS |
| | | | KDYFPEPVTVSWNSGALT | | TLYLQMNSLR | | PTTFGQGTKVEI | | RFSGSGSG |
| | | | SGVHTFPAVLQSSGLYSLS | | AEDTAVYYC | | KRTVAAPSVFIF | | TDFTLTIS |
| | | | SVVTVPSSSLGTQTYICNV | | ARWGDEGFDI | | PPSDEQLKSGTA | | SLQPEDFA |
| | | | NHKPSNTKVDKRVEPKSC | | WGQGTLVTV | | SVVCLLNNFYPR | | TYYCQQY |
| | | | DKTHTCPPCPAPELLGGPS | | SS | | EAKVQWKVDN | | SSFPTTFG |
| | | | VFLFPPKPKDTLMISRTPE | | | | ALQSGNSQESVT | | QGTKVEI |
| | | | VTCVVVDVSHEDPEVKFN | | | | EQDSKDSTYSLS | | K |
| | | | WYVDGVEVHNAKTKPRE | | | | STLTLSKADYEK | | |
| | | | EQYNSTYRVVSVLTVLHQ | | | | HKVYACEVTHQ | | |
| | | | DWLNGKEYKCKVSNKAL | | | | GLSSPVTKSFNR | | |
| | | | PAPIEKTISKAKGQPREPQ | | | | GEC | | |
| | | | VYTLPPSRDELTKNQVSL | | | | | | |
| | | | WCLVKGFYPSDIAVEWES | | | | | | |
| | | | NGQPENNYKTTPPVLDSD | | | | | | |
| | | | GSFFLYSKLTVDKSRWQQ | | | | | | |
| | | | GNVFSCSVMHEALHNHYT | | | | | | |
| | | | QKSLSLSPGK | | | | | | |
| EPI819 | CDH17 | 34 | EVQLVESGGGLVQPGGSL | 35 | EVQLVESGGG | 36 | DIQMTQSPSSL | 37 | DIQMTQS |
| | | | RLSCAASGFTFSSYAMSW | | LVQPGGSLRL | | SASVGDRVTITC | | PSSLSASV |
| | | | VRQTPGKGLEWVAVIDSN | | SCAASGFTFS | | RASQDISGYLN | | GDRVTITC |
| | | | GGSTYYPDTVKDRFTISRD | | SYAMSWVRQ | | WLQQKPGGAIK | | RASQDISG |
| | | | NSKNTLYLQMNSLRAEDT | | TPGKGLEWV | | RLIYTTSTLDSG | | YLNWLQQ |
| | | | AVYYCSSYTNLGAYWGQ | | AVIDSNGGST | | VPKRFSGSGSGT | | KPGGAIK |
| | | | GTLVTVSAASTKGPSVFPL | | YYPDTVKDRF | | DFTLTISSLQSED | | RLIYTTST |
| | | | APSSKSTSGGTAALGCLV | | TISRDNSKNT | | FATYYCLQYAS | | LDSGVPK |
| | | | KDYFPEPVTVSWNSGALT | | LYLQMNSLR | | SPFTFGGGTKVE | | RFSGSGSG |
| | | | SGVHTFPAVLQSSGLYSLS | | AEDTAVYYC | | IKRTVAAPSVFIF | | TDFTLTIS |
| | | | SVVTVPSSSLGTQTYICNV | | SSYTNLGAY | | PPSDEQLKSGTA | | SLQSEDFA |
| | | | NHKPSNTKVDKKVEPKSC | | WGQGTLVTV | | SVVCLLNNFYPR | | TYYCLQY |
| | | | DKTHTCPPCPAPELLGGPS | | SA | | EAKVQWKVDN | | ASSPFTFG |
| | | | VFLFPPKPKDTLMISRTPE | | | | ALQSGNSQESVT | | GGTKVEI |
| | | | VTCVVVDVSHEDPEVKFN | | | | EQDSKDSTYSLS | | K |
| | | | WYVDGVEVHNAKTKPRE | | | | STLTLSKADYEK | | |
| | | | EQYNSTYRVVSVLTVLHQ | | | | HKVYACEVTHQ | | |
| | | | DWLNGKEYKCKVSNKAL | | | | GLSSPVTKSFNR | | |
| | | | PAPIEKTISKAKGQPREPQ | | | | GEC | | |
| | | | VYTLPPSRDELTKNQVSL | | | | | | |
| | | | WCLVKGFYPSDIAVEWES | | | | | | |
| | | | NGQPENNYKTTPPVLDSD | | | | | | |
| | | | GSFFLYSKLTVDKSRWQQ | | | | | | |
| | | | GNVFSCSVMHEALHNHYT | | | | | | |
| | | | QKSLSLSPGK | | | | | | |
| EPI820 | CDH17 | 38 | QVQLVESGGGVVQPGRSL | 39 | QVQLVESGG | 40 | DIVMTQTPLSL | 41 | DIVMTQT |
| | | | RLSCAASGFTFSDYYMYW | | GVVQPGRSLR | | SVTPGQPASISC | | PLSLSVTP |
| | | | VRQAPGKGLEWVASISFD | | LSCAASGFTF | | RSSQSIVHSNGN | | GQPASISC |
| | | | GTYTYYTDRVKGRFTISR | | SDYYMYWVR | | TYLEWYLQKPG | | RSSQSIVH |
| | | | DNSKNTLYLQMNSLRAED | | QAPGKGLEW | | QSPQLLIYKVSN | | SNGNTYL |
| | | | TAVYYCARDRPAWFPYW | | VASISFDGTY | | RFSGVPDRFSGS | | EWYLQKP |
| | | | GQGTLVTVSAASTKGPSV | | TYYTDRVKG | | GSGTDFTLKISR | | GQSPQLLI |
| | | | FPLAPSSKSTSGGTAALGC | | RFTISRDNSK | | VEAEDVGVYYC | | YKVSNRF |
| | | | LVKDYFPEPVTVSWNSGA | | NTLYLQMNS | | FQGSHVPLTFGA | | SGVPDRFS |
| | | | LTSGVHTFPAVLQSSGLYS | | LRAEDTAVY | | GTKLELKRTVA | | GSGSGTD |
| | | | LSSVVTVPSSSLGTQTYIC | | YCARDRPAW | | APSVFIFPPSDEQ | | FTLKISRV |
| | | | NVNHKPSNTKVDKKVEPK | | FPYWGQGTL | | LKSGTASVVCLL | | EAEDVGV |
| | | | SCDKTHTCPPCPAPELLGG | | VTVSA | | NNFYPREAKVQ | | YYCFQGS |
| | | | PSVFLFPPKPKDTLMISRTP | | | | WKVDNALQSG | | HVPLTFG |
| | | | EVTCVVVDVSHEDPEVKF | | | | NSQESVTEQDSK | | AGTKLEL |
| | | | NWYVDGVEVHNAKTKPR | | | | DSTYSLSSTLTL | | K |
| | | | EEQYNSTYRVVSVLTVLH | | | | SKADYEKHKVY | | |
| | | | QDWLNGKEYKCKVSNKA | | | | ACEVTHQGLSSP | | |
| | | | LPAPIEKTISKAKGQPREPQ | | | | VTKSFNRGEC | | |
| | | | VYTLPPSRDELTKNQVSL | | | | | | |
| | | | WCLVKGFYPSDIAVEWES | | | | | | |
| | | | NGQPENNYKTTPPVLDSD | | | | | | |
| | | | GSFFLYSKLTVDKSRWQQ | | | | | | |
| | | | GNVFSCSVMHEALHNHYT | | | | | | |
| | | | QKSLSLSPGK | | | | | | |
| EPI821 | CDH17 | 42 | EVQLLETGGGVVKPGGSL | 43 | EVQLLETGGG | 44 | DVVLTQTPLSL | 45 | DVVLTQT |
| | | | KLSCAASGFTFSNYGMSW | | VVKPGGSLKL | | PVTLGDQASISC | | PLSLPVTL |
| | | | VRQTPEKRLEWVAAINRD | | SCAASGFTFS | | RSSQSLLHSNGN | | GDQASISC |
| | | | GGTTYYTDNVKGRFTISR | | NYGMSWVRQ | | TYLHWYLLKPG | | RSSQSLLH |
| | | | DNAKNSLYLQMSSLRSED | | TPEKRLEWV | | QSPKLLIYKVSN | | SNGNTYL |
| | | | TALYYCARQFLLWDGWY | | AAINRDGGTT | | RFSGVPDRFSGS | | HWYLLKP |
| | | | FDVWGAGTTVTVSSASTK | | YYTDNVKGR | | GSGTDFTLKITR | | GQSPKLLI |
| | | | GPSVFPLAPSSKSTSGGTA | | FTISRDNAKN | | VEAEDLGVYFC | | YKVSNRF |
| | | | ALGCLVKDYFPEPVTVSW | | SLYLQMSSLR | | SQSTHVLTFGAG | | SGVPDRFS |
| | | | NSGALTSGVHTFPAVLQSS | | SEDTALYYCA | | TKLELKRTVAA | | GSGSGTD |
| | | | GLYSLSSVVTVPSSSLGTQ | | RQFLLWDGW | | PSVFIFPPSDEQL | | FTLKITRV |
| | | | TYICNVNHKPSNTKVDKK | | YFDVWGAGT | | KSGTASVVCLL | | EAEDLGV |
| | | | VEPKSCDKTHTCPPCPAPE | | TVTVSS | | NNFYPREAKVQ | | YFCSQST |
| | | | LLGGPSVFLFPPKPKDTLM | | | | WKVDNALQSG | | HVLTFGA |
| | | | ISRTPEVTCVVVDVSHEDP | | | | NSQESVTEQDSK | | GTKLELK |
| | | | EVKFNWYVDGVEVHNAK | | | | DSTYSLSSTLTL | | |
| | | | TKPREEQYNSTYRVVSVL | | | | SKADYEKHKVY | | |
| | | | TVLHQDWLNGKEYKCKV | | | | ACEVTHQGLSSP | | |
| | | | SNKALPAPIEKTISKAKGQ | | | | VTKSFNRGEC | | |
| | | | PREPQVYTLPPSRDELTKN | | | | | | |
| | | | QVSLWCLVKGFYPSDIAV | | | | | | |
| | | | EWESNGQPENNYKTTPPV | | | | | | |
| | | | LDSDGSFFLYSKLTVDKSR | | | | | | |
| | | | WQQGNVFSCSVMHEALH | | | | | | |
| | | | NHYTQKSLSLSPGK | | | | | | |
| EPI822 | CDH17 | 46 | EVQLQQSVAELVKPGASV | 47 | EVQLQQSVAE | 48 | DIVMSQSPSSL | 49 | DIVMSQSP |
| | | | KMSCKVSGYTLTDHTIHW | | LVKPGASVK | | AVSVGEKVTMS | | SSLAVSV |
| | | | MKQRPEQGLEWIGYIYPR | | MSCKVSGYT | | CKSSQSLLHSSN | | GEKVTMS |
| | | | DGITGYNEKFKGKATLTA | | LTDHTIHWM | | QKNYLAWYQQ | | CKSSQSLL |
| | | | DTSSSTAYMQLNSLTSEDS | | KQRPEQGLE | | KPGQSPKVLIY | | HSSNQKN |
| | | | AVYFCARWGYSYRNYAY | | WIGYIYPRDG | | WASTRESGVPD | | YLAWYQ |
| | | | YYDYWGQGTTLTVSSAST | | ITGYNEKFKG | | RFTGSGSGTDFT | | QKPGQSP |
| | | | KGPSVFPLAPSSKSTSGGT | | KATLTADTSS | | LTITSVKSEDLA | | KVLIYWA |
| | | | AALGCLVKDYFPEPVTVS | | STAYMQLNSL | | VYYCQQYYSYP | | STRESGVP |
| | | | WNSGALTSGVHTFPAVLQ | | TSEDSAVYFC | | WTFGGGTRLEIK | | DRFTGSG |
| | | | SSGLYSLSSVVTVPSSSLG | | ARWGYSYRN | | RTVAAPSVFIFPP | | SGTDFTLT |
| | | | TQTYICNVNHKPSNTKVD | | YAYYYDYWG | | SDEQLKSGTASV | | TSVKSED |
| | | | KKVEPKSCDKTHTCPPCP | | QGTTLTVSS | | VCLLNNFYPRE | | LAVYYCQ |
| | | | APELLGGPSVFLFPPKPKD | | | | AKVQWKVDNA | | QYYSYPW |
| | | | TLMISRTPEVTCVVVDVSH | | | | LQSGNSQESVTE | | TFGGGTR |
| | | | EDPEVKFNWYVDGVEVH | | | | QDSKDSTYSLSS | | LEIK |
| | | | NAKTKPREEQYNSTYRVV | | | | TLTLSKADYEK | | |
| | | | SVLTVLHQDWLNGKEYK | | | | HKVYACEVTHQ | | |
| | | | CKVSNKALPAPIEKTISKA | | | | GLSSPVTKSFNR | | |
| | | | KGQPREPQVYTLPPSRDEL | | | | GEC | | |
| | | | TKNQVSLWCLVKGFYPSD | | | | | | |
| | | | IAVEWESNGQPENNYKTT | | | | | | |
| | | | PPVLDSDGSFFLYSKLTVD | | | | | | |
| | | | KSRWQQGNVFSCSVMHE | | | | | | |
| | | | ALHNHYTQKSLSLSPGK | | | | | | |
| EPI823 | CDH17 | 50 | QVQLVQSGAEVKKPGSSV | 51 | QVQLVQSGA | 52 | EIVMTQSPATL | 53 | EIVMTQSP |
| | | | KVSCKASGYTFSDHTIHW | | EVKKPGSSVK | | SVSPGERATLSC | | ATLSVSPG |
| | | | VRQAPGQGLEWMGYIYPR | | VSCKASGYTF | | RASQSVLYSSNQ | | ERATLSC |
| | | | LGSTKYAEKFQGRVTITA | | SDHTIHWVRQ | | KQYLAWYQQK | | RASQSVL |
| | | | DKSTSTAYMELSSLRSEDT | | APGQGLEWM | | PGQAPRLLIYGA | | YSSNQKQ |
| | | | AVYYCARWGYYYGSSRY | | GYIYPRLGST | | STRETGIPARFS | | YLAWYQ |
| | | | YFDYWGQGTLVTVSSAST | | KYAEKFQGR | | GSGSGTEFTLTI | | QKPGQAP |
| | | | KGPSVFPLAPSSKSTSGGT | | VTITADKSTS | | SSLQSEDFAVYY | | RLLIYGAS |
| | | | AALGCLVKDYFPEPVTVS | | TAYMELSSLR | | CQQYYSYPWTF | | TRETGIPA |
| | | | WNSGALTSGVHTFPAVLQ | | SEDTAVYYC | | GQGTKLEIKRTV | | RFSGSGSG |
| | | | SSGLYSLSSVVTVPSSSLG | | ARWGYYYGS | | AAPSVFIFPPSDE | | TEFTLTIS |
| | | | TQTYICNVNHKPSNTKVD | | SRYYFDYWG | | QLKSGTASVVC | | SLQSEDFA |
| | | | KKVEPKSCDKTHTCPPCP | | QGTLVTVSS | | LLNNFYPREAK | | VYYCQQY |
| | | | APELLGGPSVFLFPPKPKD | | | | VQWKVDNALQ | | YSYPWTF |
| | | | TLMISRTPEVTCVVVDVSH | | | | SGNSQESVTEQD | | GQGTKLEI |
| | | | EDPEVKFNWYVDGVEVH | | | | SKDSTYSLSSTL | | K |
| | | | NAKTKPREEQYNSTYRVV | | | | TLSKADYEKHK | | |
| | | | SVLTVLHQDWLNGKEYK | | | | VYACEVTHQGL | | |
| | | | CKVSNKALPAPIEKTISKA | | | | SSPVTKSFNRGE | | |
| | | | KGQPREPQVYTLPPSRDEL | | | | C | | |
| | | | TKNQVSLWCLVKGFYPSD | | | | | | |
| | | | IAVEWESNGQPENNYKTT | | | | | | |
| | | | PPVLDSDGSFFLYSKLTVD | | | | | | |
| | | | KSRWQQGNVFSCSVMHE | | | | | | |
| | | | ALHNHYTQKSLSLSPGK | | | | | | |
| EPI824 | MUC1 | 54 | EVQLVESGGGLVQPGGSM | 55 | EVQLVESGGG | 56 | DIVMTQSPLSN | 57 | DIVMTQS |
| | | | RLSCVASGFPFSNYWMN | | LVQPGGSMR | | PVTPGEPASISCR | | PLSNPVTP |
| | | | WVRQAPGKGLEWVGEIRL | | LSCVASGFPF | | SSKSLLHSNGIT | | GEPASISC |
| | | | KSNQYTTHYAESVKGRFTI | | SNYWMNWV | | YFFWYLQKPGQ | | RSSKSLLH |
| | | | SRDDSKNSLYLQMNSLKT | | RQAPGKGLE | | SPQLLIYQMSNL | | SNGITYFF |
| | | | EDTAVYYCTRHYYFDYW | | WVGEIRLKSN | | ASGVPDRFSGSG | | WYLQKPG |
| | | | GQGTLVTVSSASTKGPSVF | | QYTTHYAESV | | SGTDFTLRISRV | | QSPQLLIY |
| | | | PLAPSSKSTSGGTAALGCL | | KGRFTISRDD | | EAEDVGVYYCA | | QMSNLAS |
| | | | VKDYFPEPVTVSWNSGAL | | SKNSLYLQM | | QNLELPPTFGQG | | GVPDRFS |
| | | | TSGVHTFPAVLQSSGLYSL | | NSLKTEDTAV | | TKVEIKRTVAAP | | GSGSGTD |
| | | | SSVVTVPSSSLGTQTYICN | | YYCTRHYYF | | SVFIFPPSDEQLK | | FTLRISRV |
| | | | VNHKPSNTKVDKKVEPKS | | DYWGQGTLV | | SGTASVVCLLN | | EAEDVGV |
| | | | CDKTHTCPPCPAPELLGGP | | TVSS | | NFYPREAKVQW | | YYCAQNL |
| | | | SVFLFPPKPKDTLMISRTPE | | | | KVDNALQSGNS | | ELPPTFGQ |
| | | | VTCVVVDVSHEDPEVKFN | | | | QESVTEQDSKDS | | GTKVEIK |
| | | | WYVDGVEVHNAKTKPRE | | | | TYSLSSTLTLSK | | |
| | | | EQYNSTYRVVSVLTVLHQ | | | | ADYEKHKVYAC | | |
| | | | DWLNGKEYKCKVSNKAL | | | | EVTHQGLSSPVT | | |
| | | | PAPIEKTISKAKGQPREPQ | | | | KSFNRGEC | | |
| | | | VYTLPPSRDELTKNQVSL | | | | | | |
| | | | WCLVKGFYPSDIAVEWES | | | | | | |
| | | | NGQPENNYKTTPPVLDSD | | | | | | |
| | | | GSFFLYSKLTVDKSRWQQ | | | | | | |
| | | | GNVFSCSVMHEALHNHYT | | | | | | |
| | | | QKSLSLSPGK | | | | | | |
| EPI825 | MUC1 | 58 | QVQLQQSGAEVKKPGAS | 59 | QVQLQQSGA | 60 | DIQLTQSPSSLS | 61 | DIQLTQSP |
| | | | VKVSCEASGYTFPSYVLH | | EVKKPGASV | | ASVGDRVTMTC | | SSLSASVG |
| | | | WVKQAPGQGLEWIGYINP | | KVSCEASGYT | | SASSSVSSSYLY | | DRVTMTC |
| | | | YNDGTQYNEKFKGKATLT | | FPSYVLHWV | | WYQQKPGKAPK | | SASSSVSS |
| | | | RDTSINTAYMELSRLRSDD | | KQAPGQGLE | | LWIYSTSNLASG | | SYLYWYQ |
| | | | TAVYYCARGFGGSYGFAY | | WIGYINPYND | | VPARFSGSGSGT | | QKPGKAP |
| | | | WGQGTLVTVSSASTKGPS | | GTQYNEKFK | | DFTLTISSLQPED | | KLWIYSTS |
| | | | VFPLAPSSKSTSGGTAALG | | GKATLTRDTS | | SASYFCHQWNR | | NLASGVP |
| | | | CLVKDYFPEPVTVSWNSG | | INTAYMELSR | | YPYTFGGGTRLE | | ARFSGSGS |
| | | | ALTSGVHTFPAVLQSSGLY | | LRSDDTAVY | | IKRTVAAPSVFIF | | GTDFTLTI |
| | | | SLSSVVTVPSSSLGTQTYIC | | YCARGFGGS | | PPSDEQLKSGTA | | SSLQPEDS |
| | | | NVNHKPSNTKVDKKVEPK | | YGFAYWGQG | | SVVCLLNNFYPR | | ASYFCHQ |
| | | | SCDKTHTCPPCPAPELLGG | | TLVTVSS | | EAKVQWKVDN | | WNRYPYT |
| | | | PSVFLFPPKPKDTLMISRTP | | | | ALQSGNSQESVT | | FGGGTRL |
| | | | EVTCVVVDVSHEDPEVKF | | | | EQDSKDSTYSLS | | EIK |
| | | | NWYVDGVEVHNAKTKPR | | | | STLTLSKADYEK | | |
| | | | EEQYNSTYRVVSVLTVLH | | | | HKVYACEVTHQ | | |
| | | | QDWLNGKEYKCKVSNKA | | | | GLSSPVTKSFNR | | |
| | | | LPAPIEKTISKAKGQPREPQ | | | | GEC | | |
| | | | VYTLPPSRDELTKNQVSL | | | | | | |
| | | | WCLVKGFYPSDIAVEWES | | | | | | |
| | | | NGQPENNYKTTPPVLDSD | | | | | | |
| | | | GSFFLYSKLTVDKSRWQQ | | | | | | |
| | | | GNVFSCSVMHEALHNHYT | | | | | | |
| | | | QKSLSLSPGK | | | | | | |
| EPI826 | MUC1 | 62 | QVQLVQSGAEVKKPGAS | 63 | QVQLVQSGA | 64 | DIQMTQSPSSL | 65 | DIQMTQS |
| | | | VKVSCKASGYTFSAYWIE | | EVKKPGASV | | SASVGDRVTITC | | PSSLSASV |
| | | | WVRQAPGKGLEWVGEILP | | KVSCKASGYT | | KSSQSLLYSSNQ | | GDRVTITC |
| | | | GSGNSRYNEKFKGRVTVT | | FSAYWIEWV | | KIYLAWYQQKP | | KSSQSLLY |
| | | | RDTSTNTAYMELSSLRSED | | RQAPGKGLE | | GKAPKLLIYWA | | SSNQKIYL |
| | | | TAVYYCARSYDFAWFAY | | WVGEILPGSG | | STRESGVPSRFS | | AWYQQK |
| | | | WGQGTLVTVSSASTKGPS | | NSRYNEKFKG | | GSGSGTDFTFTIS | | PGKAPKL |
| | | | VFPLAPSSKSTSGGTAALG | | RVTVTRDTST | | SLQPEDIATYYC | | LIYWAST |
| | | | CLVKDYFPEPVTVSWNSG | | NTAYMELSSL | | QQYYRYPRTFG | | RESGVPSR |
| | | | ALTSGVHTFPAVLQSSGLY | | RSEDTAVYYC | | QGTKVEIKRTV | | FSGSGSGT |
| | | | SLSSVVTVPSSSLGTQTYIC | | ARSYDFAWF | | AAPSVFIFPPSDE | | DFTFTISS |
| | | | NVNHKPSNTKVDKKVEPK | | AYWGQGTLV | | QLKSGTASVVC | | LQPEDIAT |
| | | | SCDKTHTCPPCPAPELLGG | | TVSS | | LLNNFYPREAK | | YYCQQYY |
| | | | PSVFLFPPKPKDTLMISRTP | | | | VQWKVDNALQ | | RYPRTFG |
| | | | EVTCVVVDVSHEDPEVKF | | | | SGNSQESVTEQD | | QGTKVEI |
| | | | NWYVDGVEVHNAKTKPR | | | | SKDSTYSLSSTL | | K |
| | | | EEQYNSTYRVVSVLTVLH | | | | TLSKADYEKHK | | |
| | | | QDWLNGKEYKCKVSNKA | | | | VYACEVTHQGL | | |
| | | | LPAPIEKTISKAKGQPREPQ | | | | SSPVTKSFNRGE | | |
| | | | VYTLPPSRDELTKNQVSL | | | | C | | |
| | | | WCLVKGFYPSDIAVEWES | | | | | | |
| | | | NGQPENNYKTTPPVLDSD | | | | | | |
| | | | GSFFLYSKLTVDKSRWQQ | | | | | | |
| | | | GNVFSCSVMHEALHNHYT | | | | | | |
| | | | QKSLSLSPGK | | | | | | |
| EPI827 | MUC1 | 66 | QAQLVQSGAEVVKPGAS | 67 | QAQLVQSGA | 68 | EIVLTQSPATM | 69 | EIVLTQSP |
| | | | VKMSCKASGYTFTSYNM | | EVVKPGASV | | SASPGERVTITC | | ATMSASP |
| | | | HWVKQTPGQGLEWIGYIY | | KMSCKASGY | | SAHSSVSFMHW | | GERVTITC |
| | | | PGNGATNYNQKFQGKATI | | TFTSYNMHW | | FQQKPGTSPKL | | SAHSSVSF |
| | | | TADPSSSTAYMQISSLTSE | | VKQTPGQGLE | | WIYSTSSLASGV | | MHWFQQ |
| | | | DSAVYFCARGDSVPFAYW | | WIGYIYPGNG | | PARFGGSGSGTS | | KPGTSPKL |
| | | | GQGTLVTVSAASTKGPSV | | ATNYNQKFQ | | YSLTISSMEAED | | WIYSTSSL |
| | | | FPLAPSSKSTSGGTAALGC | | GKATLTADPS | | AATYYCQQRSS | | ASGVPAR |
| | | | LVKDYFPEPVTVSWNSGA | | SSTAYMQISS | | FPLTFGAGTKLE | | FGGSGSG |
| | | | LTSGVHTFPAVLQSSGLYS | | LTSEDSAVYF | | LKRTVAAPSVFI | | TSYSLTIS |
| | | | LSSVVTVPSSSLGTQTYIC | | CARGDSVPFA | | FPPSDEQLKSGT | | SMEAEDA |
| | | | NVNHKPSNTKVDKKVEPK | | YWGQGTLVT | | ASVVCLLNNFY | | ATYYCQQ |
| | | | SCDKTHTCPPCPAPELLGG | | VSA | | PREAKVQWKVD | | RSSFPLTF |
| | | | PSVFLFPPKPKDTLMISRTP | | | | NALQSGNSQES | | GAGTKLE |
| | | | EVTCVVVDVSHEDPEVKF | | | | VTEQDSKDSTYS | | LK |
| | | | NWYVDGVEVHNAKTKPR | | | | LSSTLTLSKADY | | |
| | | | EEQYNSTYRVVSVLTVLH | | | | EKHKVYACEVT | | |
| | | | QDWLNGKEYKCKVSNKA | | | | HQGLSSPVTKSF | | |
| | | | LPAPIEKTISKAKGQPREPQ | | | | NRGEC | | |
| | | | VYTLPPSRDELTKNQVSL | | | | | | |
| | | | WCLVKGFYPSDIAVEWES | | | | | | |
| | | | NGQPENNYKTTPPVLDSD | | | | | | |
| | | | GSFFLYSKLTVDKSRWQQ | | | | | | |
| | | | GNVFSCSVMHEALHNHYT | | | | | | |
| | | | QKSLSLSPGK | | | | | | |
| EPI828 | MUC1 | 70 | EVKLVESGGGLVAPGGSL | 71 | EVKLVESGGG | 72 | DVLMTQTPLS | 73 | DVLMTQT |
| | | | KLSCAASGFTFSSYPMSW | | LVAPGGSLKL | | LPVSLGDQASIS | | PLSLPVSL |
| | | | VRQTPEKRLEWVAYINNG | | SCAASGFTFS | | CRSSQTIVHSNG | | GDQASISC |
| | | | GGNPYYPDTVKGRFTISRD | | SYPMSWVRQ | | KIYLEWYLQKP | | RSSQTIVH |
| | | | NAKNTLYLQMSSLKSEDT | | TPEKRLEWV | | GQSPKLLIYRVS | | SNGKIYLE |
| | | | AIYYCIRQYYGFDYWGQG | | AYINNGGGNP | | KRFSGVPDRFSG | | WYLQKPG |
| | | | TTLTVSSASTKGPSVFPLA | | YYPDTVKGRF | | SGSGTDFTLKIS | | QSPKLLIY |
| | | | PSSKSTSGGTAALGCLVK | | TISRDNAKNT | | RVEAEDLGVYY | | RVSKRFS |
| | | | DYFPEPVTVSWNSGALTS | | LYLQMSSLKS | | CFQGSHVPWTF | | GVPDRFS |
| | | | GVHTFPAVLQSSGLYSLSS | | EDTAIYYCIR | | GGGTKLEIKRTV | | GSGSGTD |
| | | | VVTVPSSSLGTQTYICNVN | | QYYGFDYWG | | AAPSVFIFPPSDE | | FTLKISRV |
| | | | HKPSNTKVDKKVEPKSCD | | QGTTLTVSS | | QLKSGTASVVC | | EAEDLGV |
| | | | KTHTCPPCPAPELLGGPSV | | | | LLNNFYPREAK | | YYCFQGS |
| | | | FLFPPKPKDTLMISRTPEVT | | | | VQWKVDNALQ | | HVPWTFG |
| | | | CVVVDVSHEDPEVKFNW | | | | SGNSQESVTEQD | | GGTKLEIK |
| | | | YVDGVEVHNAKTKPREEQ | | | | SKDSTYSLSSTL | | |
| | | | YNSTYRVVSVLTVLHQD | | | | TLSKADYEKHK | | |
| | | | WLNGKEYKCKVSNKALP | | | | VYACEVTHQGL | | |
| | | | APIEKTISKAKGQPREPQV | | | | SSPVTKSFNRGE | | |
| | | | YTLPPSRDELTKNQVSLW | | | | C | | |
| | | | CLVKGFYPSDIAVEWESN | | | | | | |
| | | | GQPENNYKTTPPVLDSDG | | | | | | |
| | | | SFFLYSKLTVDKSRWQQG | | | | | | |
| | | | NVFSCSVMHEALHNHYTQ | | | | | | |
| | | | KSLSLSPGK | | | | | | |
| EPI829 | LY75 | 74 | QVQLVESGGGVVQPGRSL | 75 | QVQLVESGG | 76 | EIVLTQSPATL | 77 | EIVLTQSP |
| | | | RLSCAASGFTFSNYGMYW | | GVVQPGRSLR | | SLSPGERATLSC | | ATLSLSPG |
| | | | VRQAPGKGLEWVAVIWY | | LSCAASGFTF | | RASQSVSSYLA | | ERATLSC |
| | | | DGSNKYYADSVKGRFTIS | | SNYGMYWVR | | WYQQKPGQAPR | | RASQSVSS |
| | | | RDNSKNTLYLQMNSLRAE | | QAPGKGLEW | | LLIYDASNRATG | | YLAWYQ |
| | | | DTAVYYCARDLWGWYFD | | VAVIWYDGS | | IPARFSGSGSGT | | QKPGQAP |
| | | | YWGQGTLVTVSSASTKGP | | NKYYADSVK | | DFTLTISSLEPED | | RLLIYDAS |
| | | | SVFPLAPSSKSTSGGTAAL | | GRFTISRDNS | | FAVYYCQQRRN | | NRATGIPA |
| | | | GCLVKDYFPEPVTVSWNS | | KNTLYLQMN | | WPLTFGGGTKV | | RFSGSGSG |
| | | | GALTSGVHTFPAVLQSSGL | | SLRAEDTAVY | | EIKRTVAAPSVFI | | TDFTLTIS |
| | | | YSLSSVVTVPSSSLGTQTYI | | YCARDLWGW | | FPPSDEQLKSGT | | SLEPEDFA |
| | | | CNVNHKPSNTKVDKKVEP | | YFDYWGQGT | | ASVVCLLNNFY | | VYYCQQR |
| | | | KSCDKTHTCPPCPAPELLG | | LVTVSS | | PREAKVQWKVD | | RNWPLTF |
| | | | GPSVFLFPPKPKDTLMISR | | | | NALQSGNSQES | | GGGTKVE |
| | | | TPEVTCVVVDVSHEDPEV | | | | VTEQDSKDSTYS | | IK |
| | | | KFNWYVDGVEVHNAKTK | | | | LSSTLTLSKADY | | |
| | | | PREEQYNSTYRVVSVLTV | | | | EKHKVYACEVT | | |
| | | | LHQDWLNGKEYKCKVSN | | | | HQGLSSPVTKSF | | |
| | | | KALPAPIEKTISKAKGQPR | | | | NRGEC | | |
| | | | EPQVYTLPPSRDELTKNQV | | | | | | |
| | | | SLWCLVKGFYPSDIAVEW | | | | | | |
| | | | ESNGQPENNYKTTPPVLDS | | | | | | |
| | | | DGSFFLYSKLTVDKSRWQ | | | | | | |
| | | | QGNVFSCSVMHEALHNH | | | | | | |
| | | | YTQKSLSLSPGK | | | | | | |
| EPI830 | LY75 | 78 | QVQLVESGGGVVQPGRSL | 79 | QVQLVESGG | 80 | DIQMTQSPSSL | 81 | DIQMTQS |
| | | | RLSCAASGFIFSIYGMHWV | | GVVQPGRSLR | | SASVGDRVTITC | | PSSLSASV |
| | | | RQAPGKGLEWVAVIWYD | | LSCAASGFIFS | | RASQGISSWLA | | GDRVTITC |
| | | | GSNKYYADSVKGRFTISR | | IYGMHWVRQ | | WYQQKPEKAPK | | RASQGISS |
| | | | DNSKNTLYLQMNSLRAED | | APGKGLEWV | | SLIYAASSLQSG | | WLAWYQ |
| | | | TAVYYCARAPHFDYWGQ | | AVIWYDGSN | | VPSRFSGSGSGT | | QKPEKAP |
| | | | GTLVTVSSASTKGPSVFPL | | KYYADSVKG | | DFTLTISSLQPED | | KSLIYAAS |
| | | | APSSKSTSGGTAALGCLV | | RFTISRDNSK | | FATYYCQQYNS | | SLQSGVPS |
| | | | KDYFPEPVTVSWNSGALT | | NTLYLQMNS | | YPYTFGQGTKL | | RFSGSGSG |
| | | | SGVHTFPAVLQSSGLYSLS | | LRAEDTAVY | | EIKRTVAAPSVFI | | TDFTLTIS |
| | | | SVVTVPSSSLGTQTYICNV | | YCARAPHFD | | FPPSDEQLKSGT | | SLQPEDFA |
| | | | NHKPSNTKVDKKVEPKSC | | YWGQGTLVT | | ASVVCLLNNFY | | TYYCQQY |
| | | | DKTHTCPPCPAPELLGGPS | | VSS | | PREAKVQWKVD | | NSYPYTF |
| | | | VFLFPPKPKDTLMISRTPE | | | | NALQSGNSQES | | GQGTKLEI |
| | | | VTCVVVDVSHEDPEVKFN | | | | VTEQDSKDSTYS | | K |
| | | | WYVDGVEVHNAKTKPRE | | | | LSSTLTLSKADY | | |
| | | | EQYNSTYRVVSVLTVLHQ | | | | EKHKVYACEVT | | |
| | | | DWLNGKEYKCKVSNKAL | | | | HQGLSSPVTKSF | | |
| | | | PAPIEKTISKAKGQPREPQ | | | | NRGEC | | |
| | | | VYTLPPSRDELTKNQVSL | | | | | | |
| | | | WCLVKGFYPSDIAVEWES | | | | | | |
| | | | NGQPENNYKTTPPVLDSD | | | | | | |
| | | | GSFFLYSKLTVDKSRWQQ | | | | | | |
| | | | GNVFSCSVMHEALHNHYT | | | | | | |
| | | | QKSLSLSPGK | | | | | | |
| EPI831 | LY75 | 82 | EVQLVESGGGLVKPGGSL | 83 | EVQLVESGGG | 84 | DVQMTQSPSS | 85 | DVQMTQS |
| | | | RLSCAASGFTYSNAWMS | | LVKPGGSLRL | | LSASVGDRVTIT | | PSSLSASV |
| | | | WVRQAPGKGLEWVGRIK | | SCAASGFTYS | | CRASQSISDYLS | | GDRVTITC |
| | | | SKTDGGTTDYAAPVQGRF | | NAWMSWVR | | WYQQRPGKAPN | | RASQSISD |
| | | | TISRDDSKNTLYLQMNSL | | QAPGKGLEW | | LLIYAASNLKTG | | YLSWYQQ |
| | | | KTEDTAVYYCTIFGVVSFD | | VGRIKSKTDG | | VPSRFSGSGSGT | | RPGKAPN |
| | | | YWGQGTLVTVSSASTKGP | | GTTDYAAPV | | DFTLTISTLQPED | | LLIYAASN |
| | | | SVFPLAPSSKSTSGGTAAL | | QGRFTISRDD | | FATYYCQQSYR | | LKTGVPS |
| | | | GCLVKDYFPEPVTVSWNS | | SKNTLYLQM | | SPWTFGQGTKV | | RFSGSGSG |
| | | | GALTSGVHTFPAVLQSSGL | | NSLKTEDTAV | | EIKRTVAAPSVFI | | TDFTLTIS |
| | | | YSLSSVVTVPSSSLGTQTYI | | YYCTIFGVVS | | FPPSDEQLKSGT | | TLQPEDF |
| | | | CNVNHKPSNTKVDKKVEP | | FDYWGQGTL | | ASVVCLLNNFY | | ATYYCQQ |
| | | | KSCDKTHTCPPCPAPELLG | | VTVSS | | PREAKVQWKVD | | SYRSPWT |
| | | | GPSVFLFPPKPKDTLMISR | | | | NALQSGNSQES | | FGQGTKV |
| | | | TPEVTCVVVDVSHEDPEV | | | | VTEQDSKDSTYS | | EIK |
| | | | KFNWYVDGVEVHNAKTK | | | | LSSTLTLSKADY | | |
| | | | PREEQYNSTYRVVSVLTV | | | | EKHKVYACEVT | | |
| | | | LHQDWLNGKEYKCKVSN | | | | HQGLSSPVTKSF | | |
| | | | KALPAPIEKTISKAKGQPR | | | | NRGEC | | |
| | | | EPQVYTLPPSRDELTKNQV | | | | | | |
| | | | SLWCLVKGFYPSDIAVEW | | | | | | |
| | | | ESNGQPENNYKTTPPVLDS | | | | | | |
| | | | DGSFFLYSKLTVDKSRWQ | | | | | | |
| | | | QGNVFSCSVMHEALHNH | | | | | | |
| | | | YTQKSLSLSPGK | | | | | | |
| EPI832 | CEA- | 86 | QVQLVQSGAEVKKPGAS | 87 | QVQLVQSGA | 88 | DIQMTQSPSSL | 89 | DIQMTQS |
| | CAM5 | | VKVSCKASGYTFTEFGMN | | EVKKPGASV | | SASVGDRVTITC | | PSSLSASV |
| | | | WVRQAPGQGLEWMGWIN | | KVSCKASGYT | | KASAAVGTYVA | | GDRVTITC |
| | | | TKTGEATYVEEFKGRVTF | | FTEFGMNWV | | WYQQKPGKAPK | | KASAAVG |
| | | | TTDTSTSTAYMELRSLRSD | | RQAPGQGLE | | LLIYSASYRKRG | | TYVAWY |
| | | | DTAVYYCARWDFAYYVE | | WMGWINTKT | | VPSRFSGSGSGT | | QQKPGKA |
| | | | AMDYWGQGTTVTVSSAS | | GEATYVEEFK | | DFTLTISSLQPED | | PKLLIYSA |
| | | | TKGPSVFPLAPSSKSTSGG | | GRVTFTTDTS | | FATYYCHQYYT | | SYRKRGV |
| | | | TAALGCLVKDYFPEPVTV | | TSTAYMELRS | | YPLFTFGQGTKL | | PSRFSGSG |
| | | | SWNSGALTSGVHTFPAVL | | LRSDDTAVY | | EIKRTVAAPSVFI | | SGTDFTLT |
| | | | QSSGLYSLSSVVTVPSSSL | | YCARWDFAY | | FPPSDEQLKSGT | | ISSLQPED |
| | | | GTQTYICNVNHKPSNTKV | | YVEAMDYW | | ASVVCLLNNFY | | FATYYCH |
| | | | DKKVEPKSCDKTHTCPPC | | GQGTTVTVSS | | PREAKVQWKVD | | QYYTYPL |
| | | | PAPELLGGPSVFLFPPKPK | | | | NALQSGNSQES | | FTFGQGT |
| | | | DTLMISRTPEVTCVVVDVS | | | | VTEQDSKDSTYS | | KLEIK |
| | | | HEDPEVKFNWYVDGVEV | | | | LSSTLTLSKADY | | |
| | | | HNAKTKPREEQYNSTYRV | | | | EKHKVYACEVT | | |
| | | | VSVLTVLHQDWLNGKEY | | | | HQGLSSPVTKSF | | |
| | | | KCKVSNKALPAPIEKTISK | | | | NRGEC | | |
| | | | AKGQPREPQVYTLPPSRDE | | | | | | |
| | | | LTKNQVSLWCLVKGFYPS | | | | | | |
| | | | DIAVEWESNGQPENNYKT | | | | | | |
| | | | TPPVLDSDGSFFLYSKLTV | | | | | | |
| | | | DKSRWQQGNVFSCSVMH | | | | | | |
| | | | EALHNHYTQKSLSLSPGK | | | | | | |
| EPI833 | CEA- | 90 | EVKLVESGGGLVQPGGSL | 91 | EVKLVESGGG | 92 | QTVLSQSPAIL | 93 | QTVLSQSP |
| | CAM5 | | RLSCATSGFTFTDYYMNW | | LVQPGGSLRL | | SASPGEKVTMT | | AILSASPG |
| | | | VRQPPGKALEWLGFIGNK | | SCATSGFTFT | | CRASSSVTYIHW | | EKVTMTC |
| | | | ANGYTTEYSASVKGRFTIS | | DYYMNWVR | | YQQKPGSSPKS | | RASSSVT |
| | | | RDKSQSILYLQMNTLRAE | | QPPGKALEW | | WIYATSNLASG | | YIHWYQQ |
| | | | DSATYYCTRDRGLRFYFD | | LGFIGNKANG | | VPARFSGSGSGT | | KPGSSPKS |
| | | | YWGQGTTLTVSSASTKGP | | YTTEYSASVK | | SYSLTISRVEAE | | WIYATSN |
| | | | SVFPLAPSSKSTSGGTAAL | | GRFTISRDKS | | DAATYYCQHWS | | LASGVPA |
| | | | GCLVKDYFPEPVTVSWNS | | QSILYLQMNT | | SKPPTFGGGTKL | | RFSGSGSG |
| | | | GALTSGVHTFPAVLQSSGL | | LRAEDSATYY | | EIKRTVAAPSVFI | | TSYSLTIS |
| | | | YSLSSVVTVPSSSLGTQTYI | | CTRDRGLRFY | | FPPSDEQLKSGT | | RVEAEDA |
| | | | CNVNHKPSNTKVDKKVEP | | FDYWGQGTT | | ASVVCLLNNFY | | ATYYCQH |
| | | | KSCDKTHTCPPCPAPELLG | | LTVSS | | PREAKVQWKVD | | WSSKPPTF |
| | | | GPSVFLFPPKPKDTLMISR | | | | NALQSGNSQES | | GGGTKLEI |
| | | | TPEVTCVVVDVSHEDPEV | | | | VTEQDSKDSTYS | | K |
| | | | KFNWYVDGVEVHNAKTK | | | | LSSTLTLSKADY | | |
| | | | PREEQYNSTYRVVSVLTV | | | | EKHKVYACEVT | | |
| | | | LHQDWLNGKEYKCKVSN | | | | HQGLSSPVTKSF | | |
| | | | KALPAPIEKTISKAKGQPR | | | | NRGEC | | |
| | | | EPQVYTLPPSRDELTKNQV | | | | | | |
| | | | SLWCLVKGFYPSDIAVEW | | | | | | |
| | | | ESNGQPENNYKTTPPVLDS | | | | | | |
| | | | DGSFFLYSKLTVDKSRWQ | | | | | | |
| | | | QGNVFSCSVMHEALHNH | | | | | | |
| | | | YTQKSLSLSPGK | | | | | | |
| EPI834 | CD276 | 94 | QVQLQQSGAELVKPGASV | 95 | QVQLQQSGA | 96 | DIVMTQSPATL | 97 | DIVMTQS |
| | | | KLSCKASGYTFTNYDINW | | ELVKPGASVK | | SVTPGDRVSLSC | | PATLSVTP |
| | | | VRQRPEQGLEWIGWIFPG | | LSCKASGYTF | | RASQSISDYLHW | | GDRVSLS |
| | | | DGSTQYNEKFKGKATLTT | | TNYDINWVR | | YQQKSHESPRLL | | CRASQSIS |
| | | | DTSSSTAYMQLSRLTSEDS | | QRPEQGLEWI | | IKYASQSISGIPS | | DYLHWY |
| | | | AVYFCARQTTATWFAYW | | GWIFPGDGST | | RFSGSGSGSDFT | | QQKSHES |
| | | | GQGTLVTVSAASTKGPSV | | QYNEKFKGK | | LSINSVEPEDVG | | PRLLIKYA |
| | | | FPLAPSSKSTSGGTAALGC | | ATLTTDTSSS | | VYYCQNGHSFP | | SQSISGIP |
| | | | LVKDYFPEPVTVSWNSGA | | TAYMQLSRLT | | LTFGAGTKLELK | | SRFSGSGS |
| | | | LTSGVHTFPAVLQSSGLYS | | SEDSAVYFCA | | RTVAAPSVFIFPP | | GSDFTLSI |
| | | | LSSVVTVPSSSLGTQTYIC | | RQTTATWFA | | SDEQLKSGTASV | | NSVEPEDV |
| | | | NVNHKPSNTKVDKKVEPK | | YWGQGTLVT | | VCLLNNFYPRE | | GVYYCQN |
| | | | SCDKTHTCPPCPAPELLGG | | VSA | | AKVQWKVDNA | | GHSFPLTF |
| | | | PSVFLFPPKPKDTLMISRTP | | | | LQSGNSQESVTE | | GAGTKLE |
| | | | EVTCVVVDVSHEDPEVKF | | | | QDSKDSTYSLSS | | LK |
| | | | NWYVDGVEVHNAKTKPR | | | | TLTLSKADYEK | | |
| | | | EEQYNSTYRVVSVLTVLH | | | | HKVYACEVTHQ | | |
| | | | QDWLNGKEYKCKVSNKA | | | | GLSSPVTKSFNR | | |
| | | | LPAPIEKTISKAKGQPREPQ | | | | GEC | | |
| | | | VYTLPPSRDELTKNQVSL | | | | | | |
| | | | WCLVKGFYPSDIAVEWES | | | | | | |
| | | | NGQPENNYKTTPPVLDSD | | | | | | |
| | | | GSFFLYSKLTVDKSRWQQ | | | | | | |
| | | | GNVFSCSVMHEALHNHYT | | | | | | |
| | | | QKSLSLSPGK | | | | | | |
| EPI835 | CD276 | 98 | EVQLQESGPGLVKPSETLS | 99 | EVQLQESGPG | 100 | DIQMTQSPSSL | 101 | DIQMTQS |
| | | | LTCAVTGYSITSGYSWHW | | LVKPSETLSL | | SASVGDRVTITC | | PSSLSASV |
| | | | IRQFPGNGLEWMGYIHSS | | TCAVTGYSIT | | KASQNVGFNVA | | GDRVTITC |
| | | | GSTNYNPSLKSRISISRDTS | | SGYSWHWIR | | WYQQKPGKSPK | | KASQNVG |
| | | | KNQFFLKLSSVTAADTAV | | QFPGNGLEW | | ALIYSASYRYSG | | FNVAWY |
| | | | YYCAGYDDYFEYWGQGT | | MGYIHSSGST | | VPSRFSGSGSGT | | QQKPGKS |
| | | | TVTVSSASTKGPSVFPLAP | | NYNPSLKSRIS | | DFTLTISSLQPED | | PKALIYSA |
| | | | SSKSTSGGTAALGCLVKD | | ISRDTSKNQFF | | FAEYFCQQYNW | | SYRYSGV |
| | | | YFPEPVTVSWNSGALTSG | | LKLSSVTAAD | | YPFTFGQGTKLE | | PSRFSGSG |
| | | | VHTFPAVLQSSGLYSLSSV | | TAVYYCAGY | | IKRTVAAPSVFIF | | SGTDFTLT |
| | | | VTVPSSSLGTQTYICNVNH | | DDYFEYWGQ | | PPSDEQLKSGTA | | ISSLQPED |
| | | | KPSNTKVDKKVEPKSCDK | | GTTVTVSS | | SVVCLLNNFYPR | | FAEYFCQ |
| | | | THTCPPCPAPELLGGPSVF | | | | EAKVQWKVDN | | QYNWYPF |
| | | | LFPPKPKDTLMISRTPEVT | | | | ALQSGNSQESVT | | TFGQGTK |
| | | | CVVVDVSHEDPEVKFNW | | | | EQDSKDSTYSLS | | LEIK |
| | | | YVDGVEVHNAKTKPREEQ | | | | STLTLSKADYEK | | |
| | | | YNSTYRVVSVLTVLHQD | | | | HKVYACEVTHQ | | |
| | | | WLNGKEYKCKVSNKALP | | | | GLSSPVTKSFNR | | |
| | | | APIEKTISKAKGQPREPQV | | | | GEC | | |
| | | | YTLPPSRDELTKNQVSLW | | | | | | |
| | | | CLVKGFYPSDIAVEWESN | | | | | | |
| | | | GQPENNYKTTPPVLDSDG | | | | | | |
| | | | SFFLYSKLTVDKSRWQQG | | | | | | |
| | | | NVFSCSVMHEALHNHYTQ | | | | | | |
| | | | KSLSLSPGK | | | | | | |
| EPI836 | CD276 | 102 | QVQLVQSGAEVKKPGSSV | 103 | QVQLVQSGA | 104 | EIVLTQSPATL | 105 | EIVLTQSP |
| | | | KVSCKASGYTFTNYVMH | | EVKKPGSSVK | | SLSPGERATLSC | | ATLSLSPG |
| | | | WVRQAPGQGLEWMGYIN | | VSCKASGYTF | | RASSRLIYMHW | | ERATLSC |
| | | | PYNDDVKYNEKFKGRVTI | | TNYVMHWVR | | YQQKPGQAPRP | | RASSRLIY |
| | | | TADESTSTAYMELSSLRSE | | QAPGQGLEW | | LIYATSNLASGIP | | MHWYQQ |
| | | | DTAVYYCARWGYYGSPL | | MGYINPYND | | ARFSGSGSGTDF | | KPGQAPR |
| | | | YYFDYWGQGTLVTVSSAS | | DVKYNEKFK | | TLTISSLEPEDFA | | PLIYATSN |
| | | | TKGPSVFPLAPSSKSTSGG | | GRVTITADES | | VYYCQQWNSNP | | LASGIPAR |
| | | | TAALGCLVKDYFPEPVTV | | TSTAYMELSS | | PTFGQGTKVEIK | | FSGSGSGT |
| | | | SWNSGALTSGVHTFPAVL | | LRSEDTAVYY | | RTVAAPSVFIFPP | | DFTLTISS |
| | | | QSSGLYSLSSVVTVPSSSL | | CARWGYYGS | | SDEQLKSGTASV | | LEPEDFA |
| | | | GTQTYICNVNHKPSNTKV | | PLYYFDYWG | | VCLLNNFYPRE | | VYYCQQ |
| | | | DKKVEPKSCDKTHTCPPC | | QGTLVTVSS | | AKVQWKVDNA | | WNSNPPT |
| | | | PAPELLGGPSVFLFPPKPK | | | | LQSGNSQESVTE | | FGQGTKV |
| | | | DTLMISRTPEVTCVVVDVS | | | | QDSKDSTYSLSS | | EIK |
| | | | HEDPEVKFNWYVDGVEV | | | | TLTLSKADYEK | | |
| | | | HNAKTKPREEQYNSTYRV | | | | HKVYACEVTHQ | | |
| | | | VSVLTVLHQDWLNGKEY | | | | GLSSPVTKSFNR | | |
| | | | KCKVSNKALPAPIEKTISK | | | | GEC | | |
| | | | AKGQPREPQVYTLPPSRDE | | | | | | |
| | | | LTKNQVSLWCLVKGFYPS | | | | | | |
| | | | DIAVEWESNGQPENNYKT | | | | | | |
| | | | TPPVLDSDGSFFLYSKLTV | | | | | | |
| | | | DKSRWQQGNVFSCSVMH | | | | | | |
| | | | EALHNHYTQKSLSLSPGK | | | | | | |
| EPI837 | MST1R | 106 | EVQLVESGGGLVQPGGSL | 107 | EVQLVESGGG | 108 | EIVLTQSPATL | 109 | EIVLTQSP |
| | | | RLSCAASGFTFSSYLMTW | | LVQPGGSLRL | | SLSPGERATLSC | | ATLSLSPG |
| | | | VRQAPGKGLEWVANIKQ | | SCAASGFTFS | | RASQSVSRYLA | | ERATLSC |
| | | | DGSEKYYVDSVKGRFTISR | | SYLMTWVRQ | | WYQQKPGQAPR | | RASQSVS |
| | | | DNAKNSLNLQMNSLRAED | | APGKGLEWV | | LLIYDASNRATG | | RYLAWY |
| | | | TAVYYCTRDGYSSGRHYG | | ANIKQDGSEK | | IPARFSGSGSGT | | QQKPGQA |
| | | | MDVWGQGTTVIVSSASTK | | YYVDSVKGR | | DFTLTISSLEPED | | PRLLIYDA |
| | | | GPSVFPLAPSSKSTSGGTA | | FTISRDNAKN | | FAVYYCQQRSN | | SNRATGIP |
| | | | ALGCLVKDYFPEPVTVSW | | SLNLQMNSLR | | WPRTFGQGTKV | | ARFSGSGS |
| | | | NSGALTSGVHTFPAVLQSS | | AEDTAVYYC | | EIKRTVAAPSVFI | | GTDFTLTI |
| | | | GLYSLSSVVTVPSSSLGTQ | | TRDGYSSGRH | | FPPSDEQLKSGT | | SSLEPEDF |
| | | | TYICNVNHKPSNTKVDKK | | YGMDVWGQ | | ASVVCLLNNFY | | AVYYCQQ |
| | | | VEPKSCDKTHTCPPCPAPE | | GTTVIVSS | | PREAKVQWKVD | | RSNWPRT |
| | | | LLGGPSVFLFPPKPKDTLM | | | | NALQSGNSQES | | FGQGTKV |
| | | | ISRTPEVTCVVVDVSHEDP | | | | VTEQDSKDSTYS | | EIK |
| | | | EVKFNWYVDGVEVHNAK | | | | LSSTLTLSKADY | | |
| | | | TKPREEQYNSTYRVVSVL | | | | EKHKVYACEVT | | |
| | | | TVLHQDWLNGKEYKCKV | | | | HQGLSSPVTKSF | | |
| | | | SNKALPAPIEKTISKAKGQ | | | | NRGEC | | |
| | | | PREPQVYTLPPSRDELTKN | | | | | | |
| | | | QVSLWCLVKGFYPSDIAV | | | | | | |
| | | | EWESNGQPENNYKTTPPV | | | | | | |
| | | | LDSDGSFFLYSKLTVDKSR | | | | | | |
| | | | WQQGNVFSCSVMHEALH | | | | | | |
| | | | NHYTQKSLSLSPGK | | | | | | |
| EPI838 | MST1R | 110 | EVQLVESGGGLVQPGGSL | 111 | EVQLVESGGG | 112 | DIQLTQSQSFV | 113 | DIQLTQSQ |
| | | | RLSCAASGFTFSRHWMSW | | LVQPGGSLRL | | STSVGDRVTVT | | SFVSTSVG |
| | | | VRQAPGKGLEWVSEINPD | | SCAASGFTFS | | CRASQNVGSSL | | DRVTVTC |
| | | | SRTINYAPSVKGRFTISRD | | RHWMSWVR | | VWYQQKPGKSP | | RASQNVG |
| | | | NAKNSLYLQMNSLRAEDT | | QAPGKGLEW | | KTLIYSASFLYS | | SSLVWYQ |
| | | | AVYYCARRVRIHYYGAM | | VSEINPDSRTI | | GVPSRFSGSGSG | | QKPGKSP |
| | | | DSWGQGTTVTVSSASTKG | | NYAPSVKGRF | | TEFTLTISSVQPE | | KTLIYSAS |
| | | | PSVFPLAPSSKSTSGGTAA | | TISRDNAKNS | | DFADYFCQQYN | | FLYSGVPS |
| | | | LGCLVKDYFPEPVTVSWN | | LYLQMNSLR | | NYPLTFGGGTK | | RFSGSGSG |
| | | | SGALTSGVHTFPAVLQSSG | | AEDTAVYYC | | VEIKRTVAAPSV | | TEFTLTIS |
| | | | LYSLSSVVTVPSSSLGTQT | | ARRVRIHYYG | | FIFPPSDEQLKSG | | SVQPEDFA |
| | | | YICNVNHKPSNTKVDKKV | | AMDSWGQGT | | TASVVCLLNNF | | DYFCQQY |
| | | | EPKSCDKTHTCPPCPAPEL | | TVTVSS | | YPREAKVQWKV | | NNYPLTF |
| | | | LGGPSVFLFPPKPKDTLMI | | | | DNALQSGNSQE | | GGGTKVE |
| | | | SRTPEVTCVVVDVSHEDP | | | | SVTEQDSKDSTY | | IK |
| | | | EVKFNWYVDGVEVHNAK | | | | SLSSTLTLSKAD | | |
| | | | TKPREEQYNSTYRVVSVL | | | | YEKHKVYACEV | | |
| | | | TVLHQDWLNGKEYKCKV | | | | THQGLSSPVTKS | | |
| | | | SNKALPAPIEKTISKAKGQ | | | | FNRGEC | | |
| | | | PREPQVYTLPPSRDELTKN | | | | | | |
| | | | QVSLWCLVKGFYPSDIAV | | | | | | |
| | | | EWESNGQPENNYKTTPPV | | | | | | |
| | | | LDSDGSFFLYSKLTVDKSR | | | | | | |
| | | | WQQGNVFSCSVMHEALH | | | | | | |
| | | | NHYTQKSLSLSPGK | | | | | | |
| EPI839 | MST1R | 114 | EVQLQQSGAELVKPGASV | 115 | EVQLQQSGAE | 116 | DIQMNQSPSSL | 117 | DIQMNQS |
| | | | KLSCTTSGFNIIDTYIHWV | | LVKPGASVKL | | SASLGDTITITCH | | PSSLSASL |
| | | | NQKPDQGLEWIGRIDPAD | | SCTTSGFNIID | | ASQNINVWLNW | | GDTITITC |
| | | | GNRKSDPKFQVKATITVD | | TYIHWVNQK | | YQQKPGNIPKLL | | HASQNIN |
| | | | TSSNTAYLQLSSLTSGDTA | | PDQGLEWIGR | | IYKASNLHTGVP | | VWLNWY |
| | | | VYYCARGYGNLNAMDSW | | IDPADGNRKS | | SRFSGSGSGTGF | | QQKPGNIP |
| | | | GQGTSVTVSSASTKGPSVF | | DPKFQVKATI | | TLTISSLQPEDIA | | KLLIYKAS |
| | | | PLAPSSKSTSGGTAALGCL | | TVDTSSNTAY | | TYYCQQGQSYP | | NLHTGVP |
| | | | VKDYFPEPVTVSWNSGAL | | LQLSSLTSGD | | LTFGGGTKLEIK | | SRFSGSGS |
| | | | TSGVHTFPAVLQSSGLYSL | | TAVYYCARG | | RTVAAPSVFIFPP | | GTGFTLTI |
| | | | SSVVTVPSSSLGTQTYICN | | YGNLNAMDS | | SDEQLKSGTASV | | SSLQPEDI |
| | | | VNHKPSNTKVDKKVEPKS | | WGQGTSVTV | | VCLLNNFYPRE | | ATYYCQQ |
| | | | CDKTHTCPPCPAPELLGGP | | SS | | AKVQWKVDNA | | GQSYPLTF |
| | | | SVFLFPPKPKDTLMISRTPE | | | | LQSGNSQESVTE | | GGGTKLEI |
| | | | VTCVVVDVSHEDPEVKFN | | | | QDSKDSTYSLSS | | K |
| | | | WYVDGVEVHNAKTKPRE | | | | TLTLSKADYEK | | |
| | | | EQYNSTYRVVSVLTVLHQ | | | | HKVYACEVTHQ | | |
| | | | DWLNGKEYKCKVSNKAL | | | | GLSSPVTKSFNR | | |
| | | | PAPIEKTISKAKGQPREPQ | | | | GEC | | |
| | | | VYTLPPSRDELTKNQVSL | | | | | | |
| | | | WCLVKGFYPSDIAVEWES | | | | | | |
| | | | NGQPENNYKTTPPVLDSD | | | | | | |
| | | | GSFFLYSKLTVDKSRWQQ | | | | | | |
| | | | GNVFSCSVMHEALHNHYT | | | | | | |
| | | | QKSLSLSPGK | | | | | | |
| EPI840 | MST1R | 118 | QVQLVQSGAEVKKPGAT | 119 | QVQLVQSGA | 120 | EIVMTQSPGTL | 121 | EIVMTQSP |
| | | | VKISCKVSGYTFTDYHMD | | EVKKPGATV | | SLSPGERATLSC | | GTLSLSPG |
| | | | WVQQAPGKGLEWMGDIN | | KISCKVSGYT | | KSSQSLLFSGNQ | | ERATLSC |
| | | | PNNGGAIYNQKFKGRVTIT | | FTDYHMDWV | | KNYLAWYQQK | | KSSQSLLF |
| | | | ADTSTDTAYMELSSLRSE | | QQAPGKGLE | | PGQAPRLLIYW | | SGNQKNY |
| | | | DTAVYYCARSHYDYAGG | | WMGDINPNN | | ASTRASGIPDRF | | LAWYQQ |
| | | | AWFAYWGQGTLVTVSRA | | GGAIYNQKFK | | SGSGSGTDFTLT | | KPGQAPR |
| | | | STKGPSVFPLAPSSKSTSG | | GRVTITADTS | | ISRLEPEDFAVY | | LLIYWAS |
| | | | GTAALGCLVKDYFPEPVT | | TDTAYMELSS | | YCQQYYSFPRTF | | TRASGIPD |
| | | | VSWNSGALTSGVHTFPAV | | LRSEDTAVYY | | GQGTKLEIKRTV | | RFSGSGSG |
| | | | LQSSGLYSLSSVVTVPSSS | | CARSHYDYA | | AAPSVFIFPPSDE | | TDFTLTIS |
| | | | LGTQTYICNVNHKPSNTK | | GGAWFAYW | | QLKSGTASVVC | | RLEPEDFA |
| | | | VDKKVEPKSCDKTHTCPP | | GQGTLVTVSR | | LLNNFYPREAK | | VYYCQQY |
| | | | CPAPELLGGPSVFLFPPKP | | | | VQWKVDNALQ | | YSFPRTFG |
| | | | KDTLMISRTPEVTCVVVD | | | | SGNSQESVTEQD | | QGTKLEIK |
| | | | VSHEDPEVKFNWYVDGV | | | | SKDSTYSLSSTL | | |
| | | | EVHNAKTKPREEQYNSTY | | | | TLSKADYEKHK | | |
| | | | RVVSVLTVLHQDWLNGK | | | | VYACEVTHQGL | | |
| | | | EYKCKVSNKALPAPIEKTI | | | | SSPVTKSFNRGE | | |
| | | | SKAKGQPREPQVYTLPPSR | | | | C | | |
| | | | DELTKNQVSLWCLVKGFY | | | | | | |
| | | | PSDIAVEWESNGQPENNY | | | | | | |
| | | | KTTPPVLDSDGSFFLYSKL | | | | | | |
| | | | TVDKSRWQQGNVFSCSV | | | | | | |
| | | | MHEALHNHYTQKSLSLSP | | | | | | |
| | | | GK | | | | | | |
| EPI841 | MSLN | 122 | QVELVQSGAEVKKPGESL | 123 | QVELVQSGAE | 124 | DIALTQPASVS | 125 | DIALTQPA |
| | | | KISCKGSGYSFTSYWIGW | | VKKPGESLKI | | GSPGQSITISCTG | | SVSGSPG |
| | | | VRQAPGKGLEWMGIIDPG | | SCKGSGYSFT | | TSSDIGGYNSVS | | QSITISCT |
| | | | DSRTRYSPSFQGQVTISAD | | SYWIGWVRQ | | WYQQHPGKAPK | | GTSSDIGG |
| | | | KSISTAYLQWSSLKASDTA | | APGKGLEWM | | LMIYGVNNRPS | | YNSVSWY |
| | | | MYYCARGQLYGGTYMDG | | GIIDPGDSRTR | | GVSNRFSGSKSG | | QQHPGKA |
| | | | WGQGTLVTVSSASTKGPS | | YSPSFQGQVT | | NTASLTISGLQA | | PKLMIYG |
| | | | VFPLAPSSKSTSGGTAALG | | ISADKSISTAY | | EDEADYYCSSY | | VNNRPSG |
| | | | CLVKDYFPEPVTVSWNSG | | LQWSSLKASD | | DIESATPVFGGG | | VSNRFSGS |
| | | | ALTSGVHTFPAVLQSSGLY | | TAMYYCARG | | TKLTVLRTVAA | | KSGNTAS |
| | | | SLSSVVTVPSSSLGTQTYIC | | QLYGGTYMD | | PSVFIFPPSDEQL | | LTISGLQA |
| | | | NVNHKPSNTKVDKKVEPK | | GWGQGTLVT | | KSGTASVVCLL | | EDEADYY |
| | | | SCDKTHTCPPCPAPELLGG | | VSS | | NNFYPREAKVQ | | CSSYDIES |
| | | | PSVFLFPPKPKDTLMISRTP | | | | WKVDNALQSG | | ATPVFGG |
| | | | EVTCVVVDVSHEDPEVKF | | | | NSQESVTEQDSK | | GTKLTVL |
| | | | NWYVDGVEVHNAKTKPR | | | | DSTYSLSSTLTL | | |
| | | | EEQYNSTYRVVSVLTVLH | | | | SKADYEKHKVY | | |
| | | | QDWLNGKEYKCKVSNKA | | | | ACEVTHQGLSSP | | |
| | | | LPAPIEKTISKAKGQPREPQ | | | | VTKSFNRGEC | | |
| | | | VYTLPPSRDELTKNQVSL | | | | | | |
| | | | WCLVKGFYPSDIAVEWES | | | | | | |
| | | | NGQPENNYKTTPPVLDSD | | | | | | |
| | | | GSFFLYSKLTVDKSRWQQ | | | | | | |
| | | | GNVFSCSVMHEALHNHYT | | | | | | |
| | | | QKSLSLSPGK | | | | | | |
| EPI842 | MSLN | 126 | QVQLQQSGPELEKPGASV | 127 | QVQLQQSGPE | 128 | DIELTQSPAIM | 129 | DIELTQSP |
| | | | KISCKASGYSFTGYTMNW | | LEKPGASVKI | | SASPGEKVTMT | | AIMSASPG |
| | | | VKQSHGKSLEWIGLITPYN | | SCKASGYSFT | | CSASSSVSYMH | | EKVTMTC |
| | | | GASSYNQKFRGKATLTVD | | GYTMNWVK | | WYQQKSGTSPK | | SASSSVSY |
| | | | KSSSTAYMDLLSLTSEDSA | | QSHGKSLEWI | | RWIYDTSKLAS | | MHWYQQ |
| | | | VYFCARGGYDGRGFDYW | | GLITPYNGAS | | GVPGRFSGSGSG | | KSGTSPK |
| | | | GSGTPVTVSSASTKGPSVF | | SYNQKFRGK | | NSYSLTISSVEA | | RWIYDTS |
| | | | PLAPSSKSTSGGTAALGCL | | ATLTVDKSSS | | EDDATYYCQQ | | KLASGVP |
| | | | VKDYFPEPVTVSWNSGAL | | TAYMDLLSLT | | WSKHPLTFGSG | | GRFSGSGS |
| | | | TSGVHTFPAVLQSSGLYSL | | SEDSAVYFCA | | TKVEIKRTVAAP | | GNSYSLTI |
| | | | SSVVTVPSSSLGTQTYICN | | RGGYDGRGF | | SVFIFPPSDEQLK | | SSVEAED |
| | | | VNHKPSNTKVDKKVEPKS | | DYWGSGTPV | | SGTASVVCLLN | | DATYYCQ |
| | | | CDKTHTCPPCPAPELLGGP | | TVSS | | NFYPREAKVQW | | QWSKHPL |
| | | | SVFLFPPKPKDTLMISRTPE | | | | KVDNALQSGNS | | TFGSGTK |
| | | | VTCVVVDVSHEDPEVKFN | | | | QESVTEQDSKDS | | VEIK |
| | | | WYVDGVEVHNAKTKPRE | | | | TYSLSSTLTLSK | | |
| | | | EQYNSTYRVVSVLTVLHQ | | | | ADYEKHKVYAC | | |
| | | | DWLNGKEYKCKVSNKAL | | | | EVTHQGLSSPVT | | |
| | | | PAPIEKTISKAKGQPREPQ | | | | KSFNRGEC | | |
| | | | VYTLPPSRDELTKNQVSL | | | | | | |
| | | | WCLVKGFYPSDIAVEWES | | | | | | |
| | | | NGQPENNYKTTPPVLDSD | | | | | | |
| | | | GSFFLYSKLTVDKSRWQQ | | | | | | |
| | | | GNVFSCSVMHEALHNHYT | | | | | | |
| | | | QKSLSLSPGK | | | | | | |
| EPI843 | MSLN | 130 | QVHLVESGGGVVQPGRSL | 131 | QVHLVESGG | 132 | EIVLTQSPATL | 133 | EIVLTQSP |
| | | | RLSCVASGITFRIYGMHW | | GVVQPGRSLR | | SLSPGERATLSC | | ATLSLSPG |
| | | | VRQAPGKGLEWVAVLWY | | LSCVASGITFR | | RASQSVSSYLA | | ERATLSC |
| | | | DGSHEYYADSVKGRFTISR | | IYGMHWVRQ | | WYQQKPGQAPR | | RASQSVSS |
| | | | DNSKNTLYLQMNSLRAED | | APGKGLEWV | | LLIYDASNRATG | | YLAWYQ |
| | | | TAIYYCARDGDYYDSGSP | | AVLWYDGSH | | IPARFSGSGSGT | | QKPGQAP |
| | | | LDYWGQGTLVTVSSASTK | | EYYADSVKG | | DFTLTISSLEPED | | RLLIYDAS |
| | | | GPSVFPLAPSSKSTSGGTA | | RFTISRDNSK | | FAVYYCQQRSN | | NRATGIPA |
| | | | ALGCLVKDYFPEPVTVSW | | NTLYLQMNS | | WPLTFGGGTKV | | RFSGSGSG |
| | | | NSGALTSGVHTFPAVLQSS | | LRAEDTAIYY | | EIKRTVAAPSVFI | | TDFTLTIS |
| | | | GLYSLSSVVTVPSSSLGTQ | | CARDGDYYD | | FPPSDEQLKSGT | | SLEPEDFA |
| | | | TYICNVNHKPSNTKVDKK | | SGSPLDYWG | | ASVVCLLNNFY | | VYYCQQR |
| | | | VEPKSCDKTHTCPPCPAPE | | QGTLVTVSS | | PREAKVQWKVD | | SNWPLTF |
| | | | LLGGPSVFLFPPKPKDTLM | | | | NALQSGNSQES | | GGGTKVE |
| | | | ISRTPEVTCVVVDVSHEDP | | | | VTEQDSKDSTYS | | IK |
| | | | EVKFNWYVDGVEVHNAK | | | | LSSTLTLSKADY | | |
| | | | TKPREEQYNSTYRVVSVL | | | | EKHKVYACEVT | | |
| | | | TVLHQDWLNGKEYKCKV | | | | HQGLSSPVTKSF | | |
| | | | SNKALPAPIEKTISKAKGQ | | | | NRGEC | | |
| | | | PREPQVYTLPPSRDELTKN | | | | | | |
| | | | QVSLWCLVKGFYPSDIAV | | | | | | |
| | | | EWESNGQPENNYKTTPPV | | | | | | |
| | | | LDSDGSFFLYSKLTVDKSR | | | | | | |
| | | | WQQGNVFSCSVMHEALH | | | | | | |
| | | | NHYTQKSLSLSPGK | | | | | | |
| EPI844 | MSLN | 134 | EVQLVQSGAEVKKPGASV | 135 | EVQLVQSGAE | 136 | SYELTQPPSVS | 137 | SYELTQPP |
| | | | KVSCKASGDTFKRYYVH | | VKKPGASVK | | VSPGQTASITCS | | SVSVSPG |
| | | | WVRQAPGQGLEWMGIINP | | VSCKASGDTF | | GDKLGDKYAS | | QTASITCS |
| | | | SGVSTTYAQKFQGRVTMT | | KRYYVHWVR | | WYQQKPGQSPV | | GDKLGDK |
| | | | RDTSTSTVYMELSSLRSED | | QAPGQGLEW | | LVIYQDNRRPSG | | YASWYQ |
| | | | TAVYYCAEVRGSGFNYFG | | MGIINPSGVST | | IPERFSGSNSGN | | QKPGQSP |
| | | | MDVWGQGTLVTVSSAST | | TYAQKFQGR | | TATLTISGTQAM | | VLVIYQD |
| | | | KGPSVFPLAPSSKSTSGGT | | VTMTRDTSTS | | DEADYYCQAW | | NRRPSGIP |
| | | | AALGCLVKDYFPEPVTVS | | TVYMELSSLR | | DSDTYVFGTGT | | ERFSGSNS |
| | | | WNSGALTSGVHTFPAVLQ | | SEDTAVYYC | | KVTVLRTVAAP | | GNTATLTI |
| | | | SSGLYSLSSVVTVPSSSLG | | AEVRGSGFNY | | SVFIFPPSDEQLK | | SGTQAMD |
| | | | TQTYICNVNHKPSNTKVD | | FGMDVWGQG | | SGTASVVCLLN | | EADYYCQ |
| | | | KKVEPKSCDKTHTCPPCP | | TLVTVSS | | NFYPREAKVQW | | AWDSDTY |
| | | | APELLGGPSVFLFPPKPKD | | | | KVDNALQSGNS | | VFGTGTK |
| | | | TLMISRTPEVTCVVVDVSH | | | | QESVTEQDSKDS | | VTVL |
| | | | EDPEVKFNWYVDGVEVH | | | | TYSLSSTLTLSK | | |
| | | | NAKTKPREEQYNSTYRVV | | | | ADYEKHKVYAC | | |
| | | | SVLTVLHQDWLNGKEYK | | | | EVTHQGLSSPVT | | |
| | | | CKVSNKALPAPIEKTISKA | | | | KSFNRGEC | | |
| | | | KGQPREPQVYTLPPSRDEL | | | | | | |
| | | | TKNQVSLWCLVKGFYPSD | | | | | | |
| | | | LAVEWESNGQPENNYKTT | | | | | | |
| | | | PPVLDSDGSFFLYSKLTVD | | | | | | |
| | | | KSRWQQGNVFSCSVMHE | | | | | | |
| | | | ALHNHYTQKSLSLSPGK | | | | | | |
| EPI845 | EpCAM | 138 | QVQLVQSGPEVKKPGASV | 139 | QVQLVQSGPE | 140 | DIVMTQSPLSL | 141 | DIVMTQS |
| | | | KVSCKASGYTFTNYGMN | | VKKPGASVK | | PVTPGEPASISCR | | PLSLPVTP |
| | | | WVRQAPGQGLEWMGWIN | | VSCKASGYTF | | SSKNLLHSNGIT | | GEPASISC |
| | | | TYTGEPTYGEDFKGRFAFS | | TNYGMNWVR | | YLYWYLQKPGQ | | RSSKNLL |
| | | | LDTSASTAYMELSSLRSED | | QAPGQGLEW | | SPQLLIYQMSNL | | HSNGITYL |
| | | | TAVYFCARFGNYVDYWG | | MGWINTYTG | | ASGVPDRFSSSG | | YWYLQKP |
| | | | QGSLVTVSSASTKGPSVFP | | EPTYGEDFKG | | SGTDFTLKISRV | | GQSPQLLI |
| | | | LAPSSKSTSGGTAALGCLV | | RFAFSLDTSA | | EAEDVGVYYCA | | YQMSNLA |
| | | | KDYFPEPVTVSWNSGALT | | STAYMELSSL | | QNLEIPRTFGQG | | SGVPDRFS |
| | | | SGVHTFPAVLQSSGLYSLS | | RSEDTAVYFC | | TKVEIKRTVAAP | | SSGSGTDF |
| | | | SVVTVPSSSLGTQTYICNV | | ARFGNYVDY | | SVFIFPPSDEQLK | | TLKISRVE |
| | | | NHKPSNTKVDKKVEPKSC | | WGQGSLVTV | | SGTASVVCLLN | | AEDVGVY |
| | | | DKTHTCPPCPAPELLGGPS | | SS | | NFYPREAKVQW | | YCAQNLE |
| | | | VFLFPPKPKDTLMISRTPE | | | | KVDNALQSGNS | | IRTFGQG |
| | | | VTCVVVDVSHEDPEVKFN | | | | QESVTEQDSKDS | | TKVEIK |
| | | | WYVDGVEVHNAKTKPRE | | | | TYSLSSTLTLSK | | |
| | | | EQYNSTYRVVSVLTVLHQ | | | | ADYEKHKVYAC | | |
| | | | DWLNGKEYKCKVSNKAL | | | | EVTHQGLSSPVT | | |
| | | | PAPIEKTISKAKGQPREPQ | | | | KSFNRGEC | | |
| | | | VYTLPPSRDELTKNQVSL | | | | | | |
| | | | WCLVKGFYPSDIAVEWES | | | | | | |
| | | | NGQPENNYKTTPPVLDSD | | | | | | |
| | | | GSFFLYSKLTVDKSRWQQ | | | | | | |
| | | | GNVFSCSVMHEALHNHYT | | | | | | |
| | | | QKSLSLSPGK | | | | | | |
| EPI846 | EpCAM | 142 | QIQLVQSGPELKKPGETV | 143 | QIQLVQSGPE | 144 | DIVMTQAAFS | 145 | DIVMTQA |
| | | | KISCKASGYTFTKYGMNW | | LKKPGETVKI | | NPVTLGTSGSIS | | AFSNPVTL |
| | | | VKQAPGKGLKWMGWINT | | SCKASGYTFT | | CRSSKSLLHSNG | | GTSGSISC |
| | | | YTEEPTYGDDFKGRFAFSL | | KYGMNWVK | | ITYLYWYLQKP | | RSSKSLLH |
| | | | ETSASTANLQINNLKSEDT | | QAPGKGLKW | | GQSPQLLIYQMS | | SNGITYLY |
| | | | ATYFCARFGSAVDYWGQ | | MGWINTYTE | | NLASGVPDRESS | | WYLQKPG |
| | | | GTSVTVSSASTKGPSVFPL | | EPTYGDDFKG | | SGSGTDFTLRIS | | QSPQLLIY |
| | | | APSSKSTSGGTAALGCLV | | RFAFSLETSAS | | RVEAEDVGVYY | | QMSNLAS |
| | | | KDYFPEPVTVSWNSGALT | | TANLQINNLK | | CAQNLELPRTFG | | GVPDRESS |
| | | | SGVHTFPAVLQSSGLYSLS | | SEDTATYFCA | | GGTKLEIKRTVA | | SGSGTDFT |
| | | | SVVTVPSSSLGTQTYICNV | | RFGSAVDYW | | APSVFIFPPSDEQ | | LRISRVEA |
| | | | NHKPSNTKVDKKVEPKSC | | GQGTSVTVSS | | LKSGTASVVCLL | | EDVGVYY |
| | | | DKTHTCPPCPAPELLGGPS | | | | NNFYPREAKVQ | | CAQNLEL |
| | | | VFLFPPKPKDTLMISRTPE | | | | WKVDNALQSG | | PRTFGGG |
| | | | VTCVVVDVSHEDPEVKFN | | | | NSQESVTEQDSK | | TKLEIK |
| | | | WYVDGVEVHNAKTKPRE | | | | DSTYSLSSTLTL | | |
| | | | EQYNSTYRVVSVLTVLHQ | | | | SKADYEKHKVY | | |
| | | | DWLNGKEYKCKVSNKAL | | | | ACEVTHQGLSSP | | |
| | | | PAPIEKTISKAKGQPREPQ | | | | VTKSFNRGEC | | |
| | | | VYTLPPSRDELTKNQVSL | | | | | | |
| | | | WCLVKGFYPSDIAVEWES | | | | | | |
| | | | NGQPENNYKTTPPVLDSD | | | | | | |
| | | | GSFFLYSKLTVDKSRWQQ | | | | | | |
| | | | GNVFSCSVMHEALHNHYT | | | | | | |
| | | | QKSLSLSPGK | | | | | | |
| EPI847 | EpCAM | 146 | EVQLVQSGPGLVQPGGSV | 147 | EVQLVQSGPG | 148 | DIQMTQSPSSL | 149 | DIQMTQS |
| | | | RISCAASGYTFTNYGMNW | | LVQPGGSVRI | | SASVGDRVTITC | | PSSLSASV |
| | | | VKQAPGKGLEWMGWINT | | SCAASGYTFT | | RSTKSLLHSNGI | | GDRVTITC |
| | | | YTGESTYADSFKGRFTFSL | | NYGMNWVK | | TYLYWYQQKPG | | RSTKSLLH |
| | | | DTSASAAYLQINSLRAEDT | | QAPGKGLEW | | KAPKLLIYQMS | | SNGITYLY |
| | | | AVYYCARFAIKGDYWGQ | | MGWINTYTG | | NLASGVPSRFSS | | WYQQKP |
| | | | GTLLTVSSASTKGPSVFPL | | ESTYADSFKG | | SGSGTDFTLTISS | | GKAPKLLI |
| | | | APSSKSTSGGTAALGCLV | | RFTFSLDTSAS | | LQPEDFATYYC | | YQMSNLA |
| | | | KDYFPEPVTVSWNSGALT | | AAYLQINSLR | | AQNLEIPRTFGQ | | SGVPSRFS |
| | | | SGVHTFPAVLQSSGLYSLS | | AEDTAVYYC | | GTKVELKRTVA | | SSGSGTDF |
| | | | SVVTVPSSSLGTQTYICNV | | ARFAIKGDY | | APSVFIFPPSDEQ | | TLTISSLQ |
| | | | NHKPSNTKVDKKVEPKSC | | WGQGTLLTV | | LKSGTASVVCLL | | PEDFATY |
| | | | DKTHTCPPCPAPELLGGPS | | SS | | NNFYPREAKVQ | | YCAQNLE |
| | | | VFLFPPKPKDTLMISRTPE | | | | WKVDNALQSG | | IPRTFGQG |
| | | | VTCVVVDVSHEDPEVKFN | | | | NSQESVTEQDSK | | TKVELK |
| | | | WYVDGVEVHNAKTKPRE | | | | DSTYSLSSTLTL | | |
| | | | EQYNSTYRVVSVLTVLHQ | | | | SKADYEKHKVY | | |
| | | | DWLNGKEYKCKVSNKAL | | | | ACEVTHQGLSSP | | |
| | | | PAPIEKTISKAKGQPREPQ | | | | VTKSFNRGEC | | |
| | | | VYTLPPSRDELTKNQVSL | | | | | | |
| | | | WCLVKGFYPSDIAVEWES | | | | | | |
| | | | NGQPENNYKTTPPVLDSD | | | | | | |
| | | | GSFFLYSKLTVDKSRWQQ | | | | | | |
| | | | GNVFSCSVMHEALHNHYT | | | | | | |
| | | | QKSLSLSPGK | | | | | | |
| EPI848 | EpCAM | 150 | QVQLVQSGAEVKKPGAS | 151 | QVQLVQSGA | 152 | EIELTQSPGTLS | 153 | EIELTQSP |
| | | | VKVSCKASGGTFSSYAIS | | EVKKPGASV | | LSPGERATLSCR | | GTLSLSPG |
| | | | WVRQAPGQGLEWMGGIV | | KVSCKASGGT | | ASQSVSSSYLA | | ERATLSC |
| | | | PIFGTANYAQKFQGRVTIT | | FSSYAISWVR | | WYQQKPGQAPR | | RASQSVSS |
| | | | ADESTSTAYMELSSLRSED | | QAPGQGLEW | | LLIYGASSRATG | | SYLAWYQ |
| | | | TAVYYCARDPFLHYWGQ | | MGGIVPIFGT | | IPDRFSGSGSGT | | QKPGQAP |
| | | | GTLVTASTKGPSVFPLAPS | | ANYAQKFQG | | DFTLTISRLEPED | | RLLIYGAS |
| | | | SKSTSGGTAALGCLVKDY | | RVTITADESTS | | FAVYYCAQGEL | | SRATGIPD |
| | | | FPEPVTVSWNSGALTSGV | | TAYMELSSLR | | YPRQFGGGTKL | | RFSGSGSG |
| | | | HTFPAVLQSSGLYSLSSVV | | SEDTAVYYC | | DIRTVAAPSVFIF | | TDFTLTIS |
| | | | TVPSSSLGTQTYICNVNHK | | ARDPFLHYW | | PPSDEQLKSGTA | | RLEPEDFA |
| | | | PSNTKVDKKVEPKSCDKT | | GQGTLVT | | SVVCLLNNFYPR | | VYYCAQG |
| | | | HTCPPCPAPELLGGPSVFL | | | | EAKVQWKVDN | | ELYPRQF |
| | | | FPPKPKDTLMISRTPEVTC | | | | ALQSGNSQESVT | | GGGTKLD |
| | | | VVVDVSHEDPEVKFNWY | | | | EQDSKDSTYSLS | | |
| | | | VDGVEVHNAKTKPREEQY | | | | STLTLSKADYEK | | |
| | | | NSTYRVVSVLTVLHQDWL | | | | HKVYACEVTHQ | | |
| | | | NGKEYKCKVSNKALPAPI | | | | GLSSPVTKSFNR | | |
| | | | EKTISKAKGQPREPQVYTL | | | | GEC | | |
| | | | PPSRDELTKNQVSLWCLV | | | | | | |
| | | | KGFYPSDIAVEWESNGQP | | | | | | |
| | | | ENNYKTTPPVLDSDGSFFL | | | | | | |
| | | | YSKLTVDKSRWQQGNVFS | | | | | | |
| | | | CSVMHEALHNHYTQKSLS | | | | | | |
| | | | LSPGK | | | | | | |
| EPI849 | EpCAM | 154 | QVQLVQSGAEVKKPGSSV | 155 | QVQLVQSGA | 156 | EIVMTQSPATL | 157 | EIVMTQSP |
| | | | KVSCKASGGTFSSYAISW | | EVKKPGSSVK | | SVSPGERATLSC | | ATLSVSPG |
| | | | VRQAPGQGLEWMGGIIPIF | | VSCKASGGTF | | RASQSVSSNLA | | ERATLSC |
| | | | GTANYAQKFQGRVTITAD | | SSYAISWVRQ | | WYQQKPGQAPR | | RASQSVSS |
| | | | ESTSTAYMELSSLRSEDTA | | APGQGLEWM | | LIIYGASTTASGI | | NLAWYQ |
| | | | VYYCARGLLWNYWGQGT | | GGIIPIFGTAN | | PARFSASGSGTD | | QKPGQAP |
| | | | LVTVSSASTKGPSVFPLAP | | YAQKFQGRV | | FTLTISSLQSEDF | | RLIIYGAS |
| | | | SSKSTSGGTAALGCLVKD | | TITADESTSTA | | AVYYCQQYNN | | TTASGIPA |
| | | | YFPEPVTVSWNSGALTSG | | YMELSSLRSE | | WPPAYTFGQGT | | RFSASGSG |
| | | | VHTFPAVLQSSGLYSLSSV | | DTAVYYCAR | | KLEIKRTVAAPS | | TDFTLTIS |
| | | | VTVPSSSLGTQTYICNVNH | | GLLWNYWGQ | | VFIFPPSDEQLKS | | SLQSEDFA |
| | | | KPSNTKVDKKVEPKSCDK | | GTLVTVSS | | GTASVVCLLNN | | VYYCQQY |
| | | | THTCPPCPAPELLGGPSVF | | | | FYPREAKVQWK | | NNWPPAY |
| | | | LFPPKPKDTLMISRTPEVT | | | | VDNALQSGNSQ | | TFGQGTK |
| | | | CVVVDVSHEDPEVKFNW | | | | ESVTEQDSKDST | | LEIK |
| | | | YVDGVEVHNAKTKPREEQ | | | | YSLSSTLTLSKA | | |
| | | | YNSTYRVVSVLTVLHQD | | | | DYEKHKVYACE | | |
| | | | WLNGKEYKCKVSNKALP | | | | VTHQGLSSPVTK | | |
| | | | APIEKTISKAKGQPREPQV | | | | SFNRGEC | | |
| | | | YTLPPSRDELTKNQVSLW | | | | | | |
| | | | CLVKGFYPSDIAVEWESN | | | | | | |
| | | | GQPENNYKTTPPVLDSDG | | | | | | |
| | | | SFFLYSKLTVDKSRWQQG | | | | | | |
| | | | NVFSCSVMHEALHNHYTQ | | | | | | |
| | | | KSLSLSPGK | | | | | | |
| EPI850 | TNFRS | 158 | EVQLVESGGGLVQPGGSL | 159 | EVQLVESGGG | 160 | DIQMTQSPSSL | 161 | DIQMTQS |
| | F10B | | RLSCAASGFTFSSYVMSW | | LVQPGGSLRL | | SASVGDRVTITC | | PSSLSASV |
| | | | VRQAPGKGLEWVATISSG | | SCAASGFTFS | | KASQDVGTAVA | | GDRVTITC |
| | | | GSYTYYPDSVKGRFTISRD | | SYVMSWVRQ | | WYQQKPGKAPK | | KASQDVG |
| | | | NAKNTLYLQMNSLRAEDT | | APGKGLEWV | | LLIYWASTRHTG | | TAVAWY |
| | | | AVYYCARRGDSMITTDY | | ATISSGGSYT | | VPSRFSGSGSGT | | QQKPGKA |
| | | | WGQGTLVTVSSASTKGPS | | YYPDSVKGRF | | DFTLTISSLQPED | | PKLLIYW |
| | | | VFPLAPSSKSTSGGTAALG | | TISRDNAKNT | | FATYYCQQYSS | | ASTRHTG |
| | | | CLVKDYFPEPVTVSWNSG | | LYLQMNSLR | | YRTFGQGTKVEI | | VPSRFSGS |
| | | | ALTSGVHTFPAVLQSSGLY | | AEDTAVYYC | | KRTVAAPSVFIF | | GSGTDFT |
| | | | SLSSVVTVPSSSLGTQTYIC | | ARRGDSMITT | | PPSDEQLKSGTA | | LTISSLQP |
| | | | NVNHKPSNTKVDKKVEPK | | DYWGQGTLV | | SVVCLLNNFYPR | | EDFATYY |
| | | | SCDKTHTCPPCPAPELLGG | | TVSS | | EAKVQWKVDN | | CQQYSSY |
| | | | PSVFLFPPKPKDTLMISRTP | | | | ALQSGNSQESVT | | RTFGQGT |
| | | | EVTCVVVDVSHEDPEVKF | | | | EQDSKDSTYSLS | | KVEIK |
| | | | NWYVDGVEVHNAKTKPR | | | | STLTLSKADYEK | | |
| | | | EEQYNSTYRVVSVLTVLH | | | | HKVYACEVTHQ | | |
| | | | QDWLNGKEYKCKVSNKA | | | | GLSSPVTKSFNR | | |
| | | | LPAPIEKTISKAKGQPREPQ | | | | GEC | | |
| | | | VYTLPPSRDELTKNQVSL | | | | | | |
| | | | WCLVKGFYPSDIAVEWES | | | | | | |
| | | | NGQPENNYKTTPPVLDSD | | | | | | |
| | | | GSFFLYSKLTVDKSRWQQ | | | | | | |
| | | | GNVFSCSVMHEALHNHYT | | | | | | |
| | | | QKSLSLSPGK | | | | | | |
| EPI851 | TNFRS | 162 | EVQLVQSGGGVERPGGSL | 163 | EVQLVQSGG | 164 | SELTQDPAVSV | 165 | SELTQDP |
| | F10B | | RLSCAASGFTFDDYAMSW | | GVERPGGSLR | | ALGQTVRITCSG | | AVSVALG |
| | | | VRQAPGKGLEWVSGINW | | LSCAASGFTF | | DSLRSYYASWY | | QTVRITCS |
| | | | QGGSTGYADSVKGRVTIS | | DDYAMSWVR | | QQKPGQAPVLVI | | GDSLRSY |
| | | | RDNAKNSLYLQMNSLRAE | | QAPGKGLEW | | YGANNRPSGIPD | | YASWYQ |
| | | | DTAVYYCAKILGAGRGW | | VSGINWQGGS | | RFSGSSSGNTAS | | QKPGQAP |
| | | | YFDYWGKGTTVTVSSAST | | TGYADSVKG | | LTITGAQAEDEA | | VLVIYGA |
| | | | KGPSVFPLAPSSKSTSGGT | | RVTISRDNAK | | DYYCNSADSSG | | NNRPSGIP |
| | | | AALGCLVKDYFPEPVTVS | | NSLYLQMNSL | | NHVVFGGGTKL | | DRFSGSSS |
| | | | WNSGALTSGVHTFPAVLQ | | RAEDTAVYY | | TVLRTVAAPSVF | | GNTASLTI |
| | | | SSGLYSLSSVVTVPSSSLG | | CAKILGAGRG | | IFPPSDEQLKSGT | | TGAQAED |
| | | | TQTYICNVNHKPSNTKVD | | WYFDYWGK | | ASVVCLLNNFY | | EADYYCN |
| | | | KKVEPKSCDKTHTCPPCP | | GTTVTVSS | | PREAKVQWKVD | | SADSSGN |
| | | | APELLGGPSVFLFPPKPKD | | | | NALQSGNSQES | | HVVFGGG |
| | | | TLMISRTPEVTCVVVDVSH | | | | VTEQDSKDSTYS | | TKLTVL |
| | | | EDPEVKFNWYVDGVEVH | | | | LSSTLTLSKADY | | |
| | | | NAKTKPREEQYNSTYRVV | | | | EKHKVYACEVT | | |
| | | | SVLTVLHQDWLNGKEYK | | | | HQGLSSPVTKSF | | |
| | | | CKVSNKALPAPIEKTISKA | | | | NRGEC | | |
| | | | KGQPREPQVYTLPPSRDEL | | | | | | |
| | | | TKNQVSLWCLVKGFYPSD | | | | | | |
| | | | LAVEWESNGQPENNYKTT | | | | | | |
| | | | PPVLDSDGSFFLYSKLTVD | | | | | | |
| | | | KSRWQQGNVFSCSVMHE | | | | | | |
| | | | ALHNHYTQKSLSLSPGK | | | | | | |
| EPI852 | TNFRS | 166 | EVQLQQSGAEVVKPGASV | 167 | EVQLQQSGAE | 168 | EIVMTQSPATL | 169 | EIVMTQSP |
| | F10B | | KLSCKASGFNIKDTFIHWV | | VVKPGASVK | | SVSPGERATLSC | | ATLSVSPG |
| | | | KQAPGQGLEWIGRIDPAN | | LSCKASGFNI | | RASQSISNNLHW | | ERATLSC |
| | | | TNTKYDPKFQGKATITTDT | | KDTFIHWVK | | YQQKPGQAPRL | | RASQSISN |
| | | | SSNTAYMELSSLRSEDTAV | | QAPGQGLEWI | | LIKFASQSITGIP | | NLHWYQ |
| | | | YYCVRGLYTYYFDYWGQ | | GRIDPANTNT | | ARFSGSGSGTEF | | QKPGQAP |
| | | | GTLVTVSSASTKGPSVFPL | | KYDPKFQGK | | TLTISSLQSEDFA | | RLLIKFAS |
| | | | APSSKSTSGGTAALGCLV | | ATITTDTSSNT | | VYYCQQGNSWP | | QSITGIPA |
| | | | KDYFPEPVTVSWNSGALT | | AYMELSSLRS | | YTFGQGTKLEIK | | RFSGSGSG |
| | | | SGVHTFPAVLQSSGLYSLS | | EDTAVYYCV | | RTVAAPSVFIFPP | | TEFTLTIS |
| | | | SVVTVPSSSLGTQTYICNV | | RGLYTYYFD | | SDEQLKSGTASV | | SLQSEDFA |
| | | | NHKPSNTKVDKKVEPKSC | | YWGQGTLVT | | VCLLNNFYPRE | | VYYCQQG |
| | | | DKTHTCPPCPAPELLGGPS | | VSS | | AKVQWKVDNA | | NSWPYTF |
| | | | VFLFPPKPKDTLMISRTPE | | | | LQSGNSQESVTE | | GQGTKLEI |
| | | | VTCVVVDVSHEDPEVKFN | | | | QDSKDSTYSLSS | | K |
| | | | WYVDGVEVHNAKTKPRE | | | | TLTLSKADYEK | | |
| | | | EQYNSTYRVVSVLTVLHQ | | | | HKVYACEVTHQ | | |
| | | | DWLNGKEYKCKVSNKAL | | | | GLSSPVTKSFNR | | |
| | | | PAPIEKTISKAKGQPREPQ | | | | GEC | | |
| | | | VYTLPPSRDELTKNQVSL | | | | | | |
| | | | WCLVKGFYPSDIAVEWES | | | | | | |
| | | | NGQPENNYKTTPPVLDSD | | | | | | |
| | | | GSFFLYSKLTVDKSRWQQ | | | | | | |
| | | | GNVFSCSVMHEALHNHYT | | | | | | |
| | | | QKSLSLSPGK | | | | | | |
| EPI853 | TNFRS | 170 | QVQLQESGPGLVKPSQTL | 171 | QVQLQESGPG | 172 | EIVLTQSPGTL | 173 | EIVLTQSP |
| | F10B | | SLTCTVSGGSISSGDYFWS | | LVKPSQTLSL | | SLSPGERATLSC | | GTLSLSPG |
| | | | WIRQLPGKGLEWIGHIHNS | | TCTVSGGSISS | | RASQGISRSYLA | | ERATLSC |
| | | | GTTYYNPSLKSRVTISVDT | | GDYFWSWIR | | WYQQKPGQAPS | | RASQGISR |
| | | | SKKQFSLRLSSVTAADTA | | QLPGKGLEWI | | LLIYGASSRATG | | SYLAWYQ |
| | | | VYYCARDRGGDYYYGMD | | GHIHNSGTTY | | IPDRFSGSGSGT | | QKPGQAP |
| | | | VWGQGTTVTVSSASTKGP | | YNPSLKSRVT | | DFTLTISRLEPED | | SLLIYGAS |
| | | | SVFPLAPSSKSTSGGTAAL | | ISVDTSKKQF | | FAVYYCQQFGS | | SRATGIPD |
| | | | GCLVKDYFPEPVTVSWNS | | SLRLSSVTAA | | SPWTFGQGTKV | | RFSGSGSG |
| | | | GALTSGVHTFPAVLQSSGL | | DTAVYYCAR | | EIKRTVAAPSVFI | | TDFTLTIS |
| | | | YSLSSVVTVPSSSLGTQTYI | | DRGGDYYYG | | FPPSDEQLKSGT | | RLEPEDFA |
| | | | CNVNHKPSNTKVDKKVEP | | MDVWGQGTT | | ASVVCLLNNFY | | VYYCQQF |
| | | | KSCDKTHTCPPCPAPELLG | | VTVSS | | PREAKVQWKVD | | GSSPWTF |
| | | | GPSVFLFPPKPKDTLMISR | | | | NALQSGNSQES | | GQGTKVE |
| | | | TPEVTCVVVDVSHEDPEV | | | | VTEQDSKDSTYS | | IK |
| | | | KFNWYVDGVEVHNAKTK | | | | LSSTLTLSKADY | | |
| | | | PREEQYNSTYRVVSVLTV | | | | EKHKVYACEVT | | |
| | | | LHQDWLNGKEYKCKVSN | | | | HQGLSSPVTKSF | | |
| | | | KALPAPIEKTISKAKGQPR | | | | NRGEC | | |
| | | | EPQVYTLPPSRDELTKNQV | | | | | | |
| | | | SLWCLVKGFYPSDIAVEW | | | | | | |
| | | | ESNGQPENNYKTTPPVLDS | | | | | | |
| | | | DGSFFLYSKLTVDKSRWQ | | | | | | |
| | | | QGNVFSCSVMHEALHNH | | | | | | |
| | | | YTQKSLSLSPGK | | | | | | |
| EPI854 | TNFRS | 174 | EVQLVQSGGGVERPGGSL | 175 | EVQLVQSGG | 176 | SSELTQDPAVS | 177 | SSELTQDP |
| | F10B | | RLSCAASGFTFDDYGMSW | | GVERPGGSLR | | VALGQTVRITCQ | | AVSVALG |
| | | | VRQAPGKGLEWVSGINW | | LSCAASGFTF | | GDSLRSYYASW | | QTVRITCQ |
| | | | NGGSTGYADSVKGRVTIS | | DDYGMSWVR | | YQQKPGQAPVL | | GDSLRSY |
| | | | RDNAKNSLYLQMNSLRAE | | QAPGKGLEW | | VIYGKNNRPSGI | | YASWYQ |
| | | | DTAVYYCAKILGAGRGW | | VSGINWNGGS | | PDRFSGSSSGNT | | QKPGQAP |
| | | | YFDLWGKGTTVTVSSAST | | TGYADSVKG | | ASLTITGAQAED | | VLVIYGK |
| | | | KGPSVFPLAPSSKSTSGGT | | RVTISRDNAK | | EADYYCNSRDS | | NNRPSGIP |
| | | | AALGCLVKDYFPEPVTVS | | NSLYLQMNSL | | SGNHVVFGGGT | | DRFSGSSS |
| | | | WNSGALTSGVHTFPAVLQ | | RAEDTAVYY | | KLTVLRTVAAPS | | GNTASLTI |
| | | | SSGLYSLSSVVTVPSSSLG | | CAKILGAGRG | | VFIFPPSDEQLKS | | TGAQAED |
| | | | TQTYICNVNHKPSNTKVD | | WYFDLWGKG | | GTASVVCLLNN | | EADYYCN |
| | | | KKVEPKSCDKTHTCPPCP | | TTVTVSS | | FYPREAKVQWK | | SRDSSGN |
| | | | APELLGGPSVFLFPPKPKD | | | | VDNALQSGNSQ | | HVVFGGG |
| | | | TLMISRTPEVTCVVVDVSH | | | | ESVTEQDSKDST | | TKLTVL |
| | | | EDPEVKFNWYVDGVEVH | | | | YSLSSTLTLSKA | | |
| | | | NAKTKPREEQYNSTYRVV | | | | DYEKHKVYACE | | |
| | | | SVLTVLHQDWLNGKEYK | | | | VTHQGLSSPVTK | | |
| | | | CKVSNKALPAPIEKTISKA | | | | SFNRGEC | | |
| | | | KGQPREPQVYTLPPSRDEL | | | | | | |
| | | | TKNQVSLWCLVKGFYPSD | | | | | | |
| | | | IAVEWESNGQPENNYKTT | | | | | | |
| | | | PPVLDSDGSFFLYSKLTVD | | | | | | |
| | | | KSRWQQGNVFSCSVMHE | | | | | | |
| | | | ALHNHYTQKSLSLSPGK | | | | | | |
| EPI855 | STEAP1 | 178 | EVQLVESGGGLVQPGGSL | 179 | EVQLVESGGG | 180 | DIQMTQSPSSL | 181 | DIQMTQS |
| | | | RLSCAVSGYSITSDYAWN | | LVQPGGSLRL | | SASVGDRVTITC | | PSSLSASV |
| | | | WVRQAPGKGLEWVGYIS | | SCAVSGYSITS | | KSSQSLLYRSNQ | | GDRVTITC |
| | | | NSGSTSYNPSLKSRFTISRD | | DYAWNWVR | | KNYLAWYQQK | | KSSQSLLY |
| | | | TSKNTLYLQMNSLRAEDT | | QAPGKGLEW | | PGKAPKLLIYW | | RSNQKNY |
| | | | AVYYCARERNYDYDDYY | | VGYISNSGST | | ASTRESGVPSRF | | LAWYQQ |
| | | | YAMDYWGQGTLVTVSSA | | SYNPSLKSRF | | SGSGSGTDFTLT | | KPGKAPK |
| | | | STKGPSVFPLAPSSKSTSG | | TISRDTSKNTL | | ISSLQPEDFATY | | LLIYWAS |
| | | | GTAALGCLVKDYFPEPVT | | YLQMNSLRA | | YCQQYYNYPRT | | TRESGVPS |
| | | | VSWNSGALTSGVHTFPAV | | EDTAVYYCA | | FGQGTKVEIKRT | | RFSGSGSG |
| | | | LQSSGLYSLSSVVTVPSSS | | RERNYDYDD | | VAAPSVFIFPPSD | | TDFTLTIS |
| | | | LGTQTYICNVNHKPSNTK | | YYYAMDYW | | EQLKSGTASVV | | SLQPEDFA |
| | | | VDKKVEPKSCDKTHTCPP | | GQGTLVTVSS | | CLLNNFYPREA | | TYYCQQY |
| | | | CPAPELLGGPSVFLFPPKP | | | | KVQWKVDNAL | | YNYPRTF |
| | | | KDTLMISRTPEVTCVVVD | | | | QSGNSQESVTEQ | | GQGTKVE |
| | | | VSHEDPEVKFNWYVDGV | | | | DSKDSTYSLSST | | IK |
| | | | EVHNAKTKPREEQYNSTY | | | | LTLSKADYEKH | | |
| | | | RVVSVLTVLHQDWLNGK | | | | KVYACEVTHQG | | |
| | | | EYKCKVSNKALPAPIEKTI | | | | LSSPVTKSFNRG | | |
| | | | SKAKGQPREPQVYTLPPSR | | | | EC | | |
| | | | DELTKNQVSLWCLVKGFY | | | | | | |
| | | | PSDIAVEWESNGQPENNY | | | | | | |
| | | | KTTPPVLDSDGSFFLYSKL | | | | | | |
| | | | TVDKSRWQQGNVFSCSV | | | | | | |
| | | | MHEALHNHYTQKSLSLSP | | | | | | |
| | | | GK | | | | | | |
| EPI856 | ITGB6 | 182 | EVQLVESGGGLVQPGGSL | 183 | EVQLVESGGG | 184 | EIVLTQSPATL | 185 | EIVLTQSP |
| | | | RLSCAASGFTFSRYVMSW | | LVQPGGSLRL | | SLSPGERATLSC | | ATLSLSPG |
| | | | VRQAPGKGLEWVASISSG | | SCAASGFTFS | | SASSSVSSSYLY | | ERATLSCS |
| | | | GRMYYPDTVKGRFTISRD | | RYVMSWVRQ | | WYQQKPGQAPR | | ASSSVSSS |
| | | | NAKNSLYLQMNSLRAEDT | | APGKGLEWV | | LLIYSTSNLASGI | | YLYWYQ |
| | | | AVYYCARGSIYDGYYVFP | | ASISSGGRMY | | PARFSGSGSGTD | | QKPGQAP |
| | | | YWGQGTLVTVSSASTKGP | | YPDTVKGRFT | | FTLTISSLEPEDF | | RLLIYSTS |
| | | | SVFPLAPSSKSTSGGTAAL | | ISRDNAKNSL | | AVYYCHQWSTY | | NLASGIPA |
| | | | GCLVKDYFPEPVTVSWNS | | YLQMNSLRA | | PPTFGGGTKVEI | | RFSGSGSG |
| | | | GALTSGVHTFPAVLQSSGL | | EDTAVYYCA | | KRTVAAPSVFIF | | TDFTLTIS |
| | | | YSLSSVVTVPSSSLGTQTYI | | RGSIYDGYYV | | PPSDEQLKSGTA | | SLEPEDFA |
| | | | CNVNHKPSNTKVDKKVEP | | FPYWGQGTL | | SVVCLLNNFYPR | | VYYCHQ |
| | | | KSCDKTHTCPPCPAPELLG | | VTVSS | | EAKVQWKVDN | | WSTYPPT |
| | | | GPSVFLFPPKPKDTLMISR | | | | ALQSGNSQESVT | | FGGGTKV |
| | | | TPEVTCVVVDVSHEDPEV | | | | EQDSKDSTYSLS | | EIK |
| | | | KFNWYVDGVEVHNAKTK | | | | STLTLSKADYEK | | |
| | | | PREEQYNSTYRVVSVLTV | | | | HKVYACEVTHQ | | |
| | | | LHQDWLNGKEYKCKVSN | | | | GLSSPVTKSFNR | | |
| | | | KALPAPIEKTISKAKGQPR | | | | GEC | | |
| | | | EPQVYTLPPSRDELTKNQV | | | | | | |
| | | | SLWCLVKGFYPSDIAVEW | | | | | | |
| | | | ESNGQPENNYKTTPPVLDS | | | | | | |
| | | | DGSFFLYSKLTVDKSRWQ | | | | | | |
| | | | QGNVFSCSVMHEALHNH | | | | | | |
| | | | YTQKSLSLSPGK | | | | | | |
| EPI857 | ITGB6 | 186 | QVQLQESGPGLVKPSQTL | 187 | QVQLQESGPG | 188 | SYELTQPSSVS | 189 | SYELTQPS |
| | | | SLTCTVSGGSISSGGYYWS | | LVKPSQTLSL | | VSPGQTARITCS | | SVSVSPG |
| | | | WIRQHPGKGLEWIGYIYY | | TCTVSGGSISS | | GDVLAKKSARW | | QTARITCS |
| | | | SGRTYNNPSLKSRVTISVD | | GGYYWSWIR | | FHQKPGQAPVL | | GDVLAKK |
| | | | TSKNQFSLKLSSVTAADTA | | QHPGKGLEWI | | VIYKDSERPSGIP | | SARWFHQ |
| | | | VYYCARVATGRADYHFY | | GYIYYSGRTY | | ERFSGSSSGTTV | | KPGQAPV |
| | | | AMDVWGQGTTVTVSSAS | | NNPSLKSRVT | | TLTISGAQVEDE | | LVIYKDSE |
| | | | TKGPSVFPLAPSSKSTSGG | | ISVDTSKNQF | | AAYYCYSAADN | | RPSGIPER |
| | | | TAALGCLVKDYFPEPVTV | | SLKLSSVTAA | | NLVFGGGTKLT | | FSGSSSGT |
| | | | SWNSGALTSGVHTFPAVL | | DTAVYYCAR | | VLRTVAAPSVFI | | TVTLTISG |
| | | | QSSGLYSLSSVVTVPSSSL | | VATGRADYH | | FPPSDEQLKSGT | | AQVEDEA |
| | | | GTQTYICNVNHKPSNTKV | | FYAMDVWGQ | | ASVVCLLNNFY | | AYYCYSA |
| | | | DKKVEPKSCDKTHTCPPC | | GTTVTVSS | | PREAKVQWKVD | | ADNNLVF |
| | | | PAPELLGGPSVFLFPPKPK | | | | NALQSGNSQES | | GGGTKLT |
| | | | DTLMISRTPEVTCVVVDVS | | | | VTEQDSKDSTYS | | VL |
| | | | HEDPEVKFNWYVDGVEV | | | | LSSTLTLSKADY | | |
| | | | HNAKTKPREEQYNSTYRV | | | | EKHKVYACEVT | | |
| | | | VSVLTVLHQDWLNGKEY | | | | HQGLSSPVTKSF | | |
| | | | KCKVSNKALPAPIEKTISK | | | | NRGEC | | |
| | | | AKGQPREPQVYTLPPSRDE | | | | | | |
| | | | LTKNQVSLWCLVKGFYPS | | | | | | |
| | | | DIAVEWESNGQPENNYKT | | | | | | |
| | | | TPPVLDSDGSFFLYSKLTV | | | | | | |
| | | | DKSRWQQGNVFSCSVMH | | | | | | |
| | | | EALHNHYTQKSLSLSPGK | | | | | | |
| EPI858 | MELTF | 190 | QVQLVQSGAEVKKPGAS | 191 | QVQLVQSGA | 192 | DIQMTQSPSSL | 193 | DIQMTQS |
| | | | VKVSCKASGYTFTNYRIE | | EVKKPGASV | | SASVGDRVTITC | | PSSLSASV |
| | | | WVRQAPGQGLEWMGEIL | | KVSCKASGYT | | RASQDISNYLN | | GDRVTITC |
| | | | PRGGNTNYNEKFKGRVTF | | FTNYRIEWVR | | WYQQKPGKAPK | | RASQDISN |
| | | | TADTSTSTAYMELRSLRSD | | QAPGQGLEW | | LLIYYTSRLHSG | | YLNWYQ |
| | | | DTAVYYCARDDGYYGRF | | MGEILPRGGN | | VPSRFSGSGSGT | | QKPGKAP |
| | | | AYWGQGTLVTVSSASTKG | | TNYNEKFKG | | DYTLTISSLQPE | | KLLIYYTS |
| | | | PSVFPLAPSSKSTSGGTAA | | RVTFTADTST | | DFATYYCQQGN | | RLHSGVP |
| | | | LGCLVKDYFPEPVTVSWN | | STAYMELRSL | | TLPPTFGGGTKV | | SRFSGSGS |
| | | | SGALTSGVHTFPAVLQSSG | | RSDDTAVYY | | EIKRTVAAPSVFI | | GTDYTLTI |
| | | | LYSLSSVVTVPSSSLGTQT | | CARDDGYYG | | FPPSDEQLKSGT | | SSLQPEDF |
| | | | YICNVNHKPSNTKVDKKV | | RFAYWGQGT | | ASVVCLLNNFY | | ATYYCQQ |
| | | | EPKSCDKTHTCPPCPAPEL | | LVTVSS | | PREAKVQWKVD | | GNTLPPTF |
| | | | LGGPSVFLFPPKPKDTLMI | | | | NALQSGNSQES | | GGGTKVE |
| | | | SRTPEVTCVVVDVSHEDP | | | | VTEQDSKDSTYS | | IK |
| | | | EVKFNWYVDGVEVHNAK | | | | LSSTLTLSKADY | | |
| | | | TKPREEQYNSTYRVVSVL | | | | EKHKVYACEVT | | |
| | | | TVLHQDWLNGKEYKCKV | | | | HQGLSSPVTKSF | | |
| | | | SNKALPAPIEKTISKAKGQ | | | | NRGEC | | |
| | | | PREPQVYTLPPSRDELTKN | | | | | | |
| | | | QVSLWCLVKGFYPSDIAV | | | | | | |
| | | | EWESNGQPENNYKTTPPV | | | | | | |
| | | | LDSDGSFFLYSKLTVDKSR | | | | | | |
| | | | WQQGNVFSCSVMHEALH | | | | | | |
| | | | NHYTQKSLSLSPGK | | | | | | |
| EPI859 | MELTF | 194 | QVQLQESGPGLVKPSETLS | 195 | QVQLQESGPG | 196 | DFVMTQSPLSL | 197 | DFVMTQS |
| | | | LTCTVSGDSITSGYWNWIR | | LVKPSETLSL | | PVTLGQPASISC | | PLSLPVTL |
| | | | QPPGKGLEYIGYISDSGITY | | TCTVSGDSITS | | RASQSLVHSDG | | GQPASISC |
| | | | YNPSLKSRVTISRDTSKNQ | | GYWNWIRQP | | NTYLHWYQQRP | | RASQSLV |
| | | | YSLKLSSVTAADTAVYYC | | PGKGLEYIGY | | GQSPRLLIYRVS | | HSDGNTY |
| | | | ARRTLATYYAMDYWGQG | | ISDSGITYYNP | | NRFSGVPDRFSG | | LHWYQQ |
| | | | TLVTVSSASTKGPSVFPLA | | SLKSRVTISRD | | SGSGTDFTLKIS | | RPGQSPRL |
| | | | PSSKSTSGGTAALGCLVK | | TSKNQYSLKL | | RVEAEDVGVYY | | LIYRVSNR |
| | | | DYFPEPVTVSWNSGALTS | | SSVTAADTAV | | CSQSTHVPPTFG | | FSGVPDRF |
| | | | GVHTFPAVLQSSGLYSLSS | | YYCARRTLAT | | QGTKLEIKRTVA | | SGSGSGT |
| | | | VVTVPSSSLGTQTYICNVN | | YYAMDYWG | | APSVFIFPPSDEQ | | DFTLKISR |
| | | | HKPSNTKVDKKVEPKSCD | | QGTLVTVSS | | LKSGTASVVCLL | | VEAEDVG |
| | | | KTHTCPPCPAPELLGGPSV | | | | NNFYPREAKVQ | | VYYCSQS |
| | | | FLFPPKPKDTLMISRTPEVT | | | | WKVDNALQSG | | THVPPTFG |
| | | | CVVVDVSHEDPEVKFNW | | | | NSQESVTEQDSK | | QGTKLEIK |
| | | | YVDGVEVHNAKTKPREEQ | | | | DSTYSLSSTLTL | | |
| | | | YNSTYRVVSVLTVLHQD | | | | SKADYEKHKVY | | |
| | | | WLNGKEYKCKVSNKALP | | | | ACEVTHQGLSSP | | |
| | | | APIEKTISKAKGQPREPQV | | | | VTKSFNRGEC | | |
| | | | YTLPPSRDELTKNQVSLW | | | | | | |
| | | | CLVKGFYPSDIAVEWESN | | | | | | |
| | | | GQPENNYKTTPPVLDSDG | | | | | | |
| | | | SFFLYSKLTVDKSRWQQG | | | | | | |
| | | | NVFSCSVMHEALHNHYTQ | | | | | | |
| | | | KSLSLSPGK | | | | | | |
| EPI860 | RNF43 | 198 | QVQLVQSGAEVKKPGAS | 199 | QVQLVQSGA | 200 | DIVMTQSPDSL | 201 | DIVMTQS |
| | | | VKVSCKASGFNIKDTYIH | | EVKKPGASV | | AVSLGERATINC | | PDSLAVSL |
| | | | WVRQAPGQGLEWMGRID | | KVSCKASGFN | | RASESVDSYGNS | | GERATINC |
| | | | PANGKANYDPKFQGRVT | | IKDTYIHWVR | | FMHWYQQKPG | | RASESVD |
| | | | MTRDTSTSTVYMELSSLRS | | QAPGQGLEW | | QPPKLLIYLASN | | SYGNSFM |
| | | | EDTAVYYCALGGGYYGM | | MGRIDPANG | | LESGVPDRFSGS | | HWYQQK |
| | | | DYWGQGTLVTVSSASTKG | | KANYDPKFQ | | GSGTDFTLTISSL | | PGQPPKLL |
| | | | PSVFPLAPSSKSTSGGTAA | | GRVTMTRDT | | QAEDVAVYYCQ | | IYLASNLE |
| | | | LGCLVKDYFPEPVTVSWN | | STSTVYMELS | | QNNEDPLTFGQ | | SGVPDRFS |
| | | | SGALTSGVHTFPAVLQSSG | | SLRSEDTAVY | | GTKVEIKRTVA | | GSGSGTD |
| | | | LYSLSSVVTVPSSSLGTQT | | YCALGGGYY | | APSVFIFPPSDEQ | | FTLTISSL |
| | | | YICNVNHKPSNTKVDKKV | | GMDYWGQG | | LKSGTASVVCLL | | QAEDVAV |
| | | | EPKSCDKTHTCPPCPAPEL | | TLVTVSS | | NNFYPREAKVQ | | YYCQQNN |
| | | | LGGPSVFLFPPKPKDTLMI | | | | WKVDNALQSG | | EDPLTFG |
| | | | SRTPEVTCVVVDVSHEDP | | | | NSQESVTEQDSK | | QGTKVEI |
| | | | EVKFNWYVDGVEVHNAK | | | | DSTYSLSSTLTL | | K |
| | | | TKPREEQYNSTYRVVSVL | | | | SKADYEKHKVY | | |
| | | | TVLHQDWLNGKEYKCKV | | | | ACEVTHQGLSSP | | |
| | | | SNKALPAPIEKTISKAKGQ | | | | VTKSFNRGEC | | |
| | | | PREPQVYTLPPSRDELTKN | | | | | | |
| | | | QVSLWCLVKGFYPSDIAV | | | | | | |
| | | | EWESNGQPENNYKTTPPV | | | | | | |
| | | | LDSDGSFFLYSKLTVDKSR | | | | | | |
| | | | WQQGNVFSCSVMHEALH | | | | | | |
| | | | NHYTQKSLSLSPGK | | | | | | |
| EPI861 | RNF43 | 202 | QQQLEEYGGDLVQPEGSL | 203 | QQQLEEYGG | 204 | AEIVMTQTPSS | 205 | AEIVMTQ |
| | | | TLTCKASGLDFSSSYWMC | | DLVQPEGSLT | | KSAAVGDTVTI | | TPSSKSAA |
| | | | WVRQAPGKGLEWIACIYT | | LTCKASGLDF | | KCQASQSITSYL | | VGDTVTI |
| | | | GSSGSTSYASWAKGRFTIS | | SSSYWMCWV | | SWYQQKPGQPP | | KCQASQSI |
| | | | KTSSTTVTLQMTSLTAAD | | RQAPGKGLE | | KLLIYRASTLAS | | TSYLSWY |
| | | | TATYFCARDYDYTAYAY | | WIACIYTGSS | | GVPSRFKGSGSG | | QQKPGQP |
| | | | GIMSLWGPGTLVTVSSAST | | GSTSYASWA | | TQFTLTISDLEC | | PKLLIYRA |
| | | | KGPSVFPLAPSSKSTSGGT | | KGRFTISKTSS | | ADAATYYCQSN | | STLASGVP |
| | | | AALGCLVKDYFPEPVTVS | | TTVTLQMTSL | | YGSYSTNYGVT | | SRFKGSGS |
| | | | WNSGALTSGVHTFPAVLQ | | TAADTATYFC | | FGGGTKVEIKRT | | GTQFTLTI |
| | | | SSGLYSLSSVVTVPSSSLG | | ARDYDYTAY | | VAAPSVFIFPPSD | | SDLECAD |
| | | | TQTYICNVNHKPSNTKVD | | AYGIMSLWG | | EQLKSGTASVV | | AATYYCQ |
| | | | KKVEPKSCDKTHTCPPCP | | PGTLVTVSS | | CLLNNFYPREA | | SNYGSYS |
| | | | APELLGGPSVFLFPPKPKD | | | | KVQWKVDNAL | | TNYGVTF |
| | | | TLMISRTPEVTCVVVDVSH | | | | QSGNSQESVTEQ | | GGGTKVE |
| | | | EDPEVKFNWYVDGVEVH | | | | DSKDSTYSLSST | | IK |
| | | | NAKTKPREEQYNSTYRVV | | | | LTLSKADYEKH | | |
| | | | SVLTVLHQDWLNGKEYK | | | | KVYACEVTHQG | | |
| | | | CKVSNKALPAPIEKTISKA | | | | LSSPVTKSFNRG | | |
| | | | KGQPREPQVYTLPPSRDEL | | | | EC | | |
| | | | TKNQVSLWCLVKGFYPSD | | | | | | |
| | | | IAVEWESNGQPENNYKTT | | | | | | |
| | | | PPVLDSDGSFFLYSKLTVD | | | | | | |
| | | | KSRWQQGNVFSCSVMHE | | | | | | |
| | | | ALHNHYTQKSLSLSPGK | | | | | | |
| EPI862 | RNF43 | 206 | QEQLVESGGGLVQPEGSL | 207 | QEQLVESGGG | 208 | DVVMTQTPAS | 209 | DVVMTQT |
| | | | TLTCTASGFSFSSRYYMC | | LVQPEGSLTL | | VSEPVGGTVTIK | | PASVSEPV |
| | | | WVRQAPGKGLEWIGCIYT | | TCTASGFSFSS | | CQASQSIYSGLA | | GGTVTIK |
| | | | GSGSTYYASWAKGRVTIS | | RYYMCWVRQ | | WYQQKPGQPPK | | CQASQSIY |
| | | | KTSSTTVTLQMTSLTAAD | | APGKGLEWIG | | LLIYSASKLASG | | SGLAWYQ |
| | | | TATYFCAREAGSFNLWGP | | CIYTGSGSTY | | VPSRFKGSGSGT | | QKPGQPP |
| | | | GTLVTVSSASTKGPSVFPL | | YASWAKGRV | | EYTLTISDLECA | | KLLIYSAS |
| | | | APSSKSTSGGTAALGCLV | | TISKTSSTTVT | | DAATYYCQNYY | | KLASGVP |
| | | | KDYFPEPVTVSWNSGALT | | LQMTSLTAA | | YGISNGWTFGG | | SRFKGSGS |
| | | | SGVHTFPAVLQSSGLYSLS | | DTATYFCARE | | GTKVEIKRTVA | | GTEYTLTI |
| | | | SVVTVPSSSLGTQTYICNV | | AGSFNLWGP | | APSVFIFPPSDEQ | | SDLECAD |
| | | | NHKPSNTKVDKKVEPKSC | | GTLVTVSS | | LKSGTASVVCLL | | AATYYCQ |
| | | | DKTHTCPPCPAPELLGGPS | | | | NNFYPREAKVQ | | NYYYGIS |
| | | | VFLFPPKPKDTLMISRTPE | | | | WKVDNALQSG | | NGWTFGG |
| | | | VTCVVVDVSHEDPEVKFN | | | | NSQESVTEQDSK | | GTKVEIK |
| | | | WYVDGVEVHNAKTKPRE | | | | DSTYSLSSTLTL | | |
| | | | EQYNSTYRVVSVLTVLHQ | | | | SKADYEKHKVY | | |
| | | | DWLNGKEYKCKVSNKAL | | | | ACEVTHQGLSSP | | |
| | | | PAPIEKTISKAKGQPREPQ | | | | VTKSFNRGEC | | |
| | | | VYTLPPSRDELTKNQVSL | | | | | | |
| | | | WCLVKGFYPSDIAVEWES | | | | | | |
| | | | NGQPENNYKTTPPVLDSD | | | | | | |
| | | | GSFFLYSKLTVDKSRWQQ | | | | | | |
| | | | GNVFSCSVMHEALHNHYT | | | | | | |
| | | | QKSLSLSPGK | | | | | | |
| EPI863 | RNF43 | 210 | QVQLQESGGGLVQAGGSL | 211 | QVQLQESGG | | | | |
| | | | RLSCAASGSIFWKPVMGW | | GLVQAGGSL | | | | |
| | | | YRQAPGKEREFVAAITSGT | | RLSCAASGSIF | | | | |
| | | | NTYYADSVKGRFTISRDN | | WKPVMGWY | | | | |
| | | | AKNTVYLQMNSLKPEDTA | | RQAPGKEREF | | | | |
| | | | VYYCAVDDYDVVEYPYW | | VAAITSGTNT | | | | |
| | | | GQGTQVTVSSGGGGSDKT | | YYADSVKGR | | | | |
| | | | HTCPPCPAPELLGGPSVFL | | FTISRDNAKN | | | | |
| | | | FPPKPKDTLMISRTPEVTC | | TVYLQMNSL | | | | |
| | | | VVVDVSHEDPEVKFNWY | | KPEDTAVYY | | | | |
| | | | VDGVEVHNAKTKPREEQY | | CAVDDYDVV | | | | |
| | | | NSTYRVVSVLTVLHQDWL | | EYPYWGQGT | | | | |
| | | | NGKEYKCKVSNKALPAPI | | QVTVSS | | | | |
| | | | EKTISKAKGQPREPQVYTL | | | | | | |
| | | | PPSRDELTKNQVSLWCLV | | | | | | |
| | | | KGFYPSDIAVEWESNGQP | | | | | | |
| | | | ENNYKTTPPVLDSDGSFFL | | | | | | |
| | | | YSKLTVDKSRWQQGNVFS | | | | | | |
| | | | CSVMHEALHNHYTQKSLS | | | | | | |
| | | | LSPGKGGSHHHHHH | | | | | | |
| EPI864 | RNF128 | 212 | QVQLQESGGGLVQAGGSL | 213 | QVQLQESGG | | | | |
| | | | RLSCAASGNISVQLDMGW | | GLVQAGGSL | | | | |
| | | | YRQAPGKEREFVAAINQG | | RLSCAASGNI | | | | |
| | | | TTTYYADSVKGRFTISRDN | | SVQLDMGWY | | | | |
| | | | AKNTVYLQMNSLKPEDTA | | RQAPGKEREF | | | | |
| | | | VYYCAVYLYDIWNHPYW | | VAAINQGTTT | | | | |
| | | | GQGTQVTVSSGGGGSDKT | | YYADSVKGR | | | | |
| | | | HTCPPCPAPELLGGPSVFL | | FTISRDNAKN | | | | |
| | | | FPPKPKDTLMISRTPEVTC | | TVYLQMNSL | | | | |
| | | | VVVDVSHEDPEVKFNWY | | KPEDTAVYY | | | | |
| | | | VDGVEVHNAKTKPREEQY | | CAVYLYDIW | | | | |
| | | | NSTYRVVSVLTVLHQDWL | | NHPYWGQGT | | | | |
| | | | NGKEYKCKVSNKALPAPI | | QVTVSS | | | | |
| | | | EKTISKAKGQPREPQVYTL | | | | | | |
| | | | PPSRDELTKNQVSLWCLV | | | | | | |
| | | | KGFYPSDIAVEWESNGQP | | | | | | |
| | | | ENNYKTTPPVLDSDGSFFL | | | | | | |
| | | | YSKLTVDKSRWQQGNVFS | | | | | | |
| | | | CSVMHEALHNHYTQKSLS | | | | | | |
| | | | LSPGKGGSHHHHHH | | | | | | |
| EPI865 | RNF128 | 214 | QVQLQESGGGLVQAGGSL | 215 | QVQLQESGG | | | | |
| | | | RLSCAASGSISGGKGMGW | | GLVQAGGSL | | | | |
| | | | YRQAPGKEREFVAAIGSG | | RLSCAASGSIS | | | | |
| | | | AITYYADSVKGRFTISRDN | | GGKGMGWY | | | | |
| | | | AKNTVYLQMNSLKPEDTA | | RQAPGKEREF | | | | |
| | | | VYYCAVYTTALDEYPYW | | VAAIGSGAIT | | | | |
| | | | GQGTQVTVSSGGGGSDKT | | YYADSVKGR | | | | |
| | | | HTCPPCPAPELLGGPSVFL | | FTISRDNAKN | | | | |
| | | | FPPKPKDTLMISRTPEVTC | | TVYLQMNSL | | | | |
| | | | VVVDVSHEDPEVKFNWY | | KPEDTAVYY | | | | |
| | | | VDGVEVHNAKTKPREEQY | | CAVYTTALDE | | | | |
| | | | NSTYRVVSVLTVLHQDWL | | YPYWGQGTQ | | | | |
| | | | NGKEYKCKVSNKALPAPI | | VTVSS | | | | |
| | | | EKTISKAKGQPREPQVYTL | | | | | | |
| | | | PPSRDELTKNQVSLWCLV | | | | | | |
| | | | KGFYPSDIAVEWESNGQP | | | | | | |
| | | | ENNYKTTPPVLDSDGSFFL | | | | | | |
| | | | YSKLTVDKSRWQQGNVFS | | | | | | |
| | | | CSVMHEALHNHYTQKSLS | | | | | | |
| | | | LSPGKGGSHHHHHH | | | | | | |
| EPI866 | RNF128 | 216 | QVQLQESGGGLVQAGGSL | 217 | QVQLQESGG | | | | |
| | | | RLSCAASGNISYFLIMGWY | | GLVQAGGSL | | | | |
| | | | RQAPGKEREFVAAITRGSN | | RLSCAASGNI | | | | |
| | | | TYYADSVKGRFTISRDNA | | SYFLIMGWYR | | | | |
| | | | KNTVYLQMNSLKPEDTAV | | QAPGKEREFV | | | | |
| | | | YYCAVFSTLQYHYDTGYT | | AAITRGSNTY | | | | |
| | | | AYLTYWGQGTQVTVSSG | | YADSVKGRFT | | | | |
| | | | GGGSDKTHTCPPCPAPELL | | ISRDNAKNTV | | | | |
| | | | GGPSVFLFPPKPKDTLMIS | | YLQMNSLKPE | | | | |
| | | | RTPEVTCVVVDVSHEDPE | | DTAVYYCAV | | | | |
| | | | VKFNWYVDGVEVHNAKT | | FSTLQYHYDT | | | | |
| | | | KPREEQYNSTYRVVSVLT | | GYTAYLTYW | | | | |
| | | | VLHQDWLNGKEYKCKVS | | GQGTQVTVSS | | | | |
| | | | NKALPAPIEKTISKAKGQP | | | | | | |
| | | | REPQVYTLPPSRDELTKNQ | | | | | | |
| | | | VSLWCLVKGFYPSDIAVE | | | | | | |
| | | | WESNGQPENNYKTTPPVL | | | | | | |
| | | | DSDGSFFLYSKLTVDKSR | | | | | | |
| | | | WQQGNVFSCSVMHEALH | | | | | | |
| | | | NHYTQKSLSLSPGKGGSH | | | | | | |
| | | | HHHHH | | | | | | |
| EPI867 | CD71 | 218 | QVQLQQSGPDLVKPGASV | 219 | QVQLQQSGP | 220 | DILLTQSPAILS | 221 | DILLTQSP |
| | | | RISCKASGYTFAGHYVHW | | DLVKPGASVR | | VSPGDRVSFSCR | | AILSVSPG |
| | | | VKQRPGRGLEWIGWIFPG | | ISCKASGYTF | | ASQSIGTSIHWY | | DRVSFSC |
| | | | KVNTKYNEKFKGKATLTA | | AGHYVHWVK | | QQRTDGSPRLLI | | RASQSIGT |
| | | | DKSSSTAYMQLSSLTSEDS | | QRPGRGLEWI | | KYASESISGIPSR | | SIHWYQQ |
| | | | AVYFCARVGYDYPYYFD | | GWIFPGKVNT | | FSGSGSGTDFTL | | RTDGSPR |
| | | | YWGQGTTLTVSSASTKGP | | KYNEKFKGK | | SINSVESEDVAD | | LLIKYASE |
| | | | SVFPLAPSSKSTSGGTAAL | | ATLTADKSSS | | YYCQQSSSWPF | | SISGIPSR |
| | | | GCLVKDYFPEPVTVSWNS | | TAYMQLSSLT | | TFGSGTKLEIKR | | FSGSGSGT |
| | | | GALTSGVHTFPAVLQSSGL | | SEDSAVYFCA | | TVAAPSVFIFPPS | | DFTLSINS |
| | | | YSLSSVVTVPSSSLGTQTYI | | RVGYDYPYY | | DEQLKSGTASV | | VESEDVA |
| | | | CNVNHKPSNTKVDKKVEP | | FDYWGQGTT | | VCLLNNFYPRE | | DYYCQQS |
| | | | KSCDKTHTCPPCPAPELLG | | LTVSS | | AKVQWKVDNA | | SSWPFTFG |
| | | | GPSVFLFPPKPKDTLMISR | | | | LQSGNSQESVTE | | SGTKLEIK |
| | | | TPEVTCVVVDVSHEDPEV | | | | QDSKDSTYSLSS | | |
| | | | KFNWYVDGVEVHNAKTK | | | | TLTLSKADYEK | | |
| | | | PREEQYNSTYRVVSVLTV | | | | HKVYACEVTHQ | | |
| | | | LHQDWLNGKEYKCKVSN | | | | GLSSPVTKSFNR | | |
| | | | KALPAPIEKTISKAKGQPR | | | | GEC | | |
| | | | EPQVYTLPPSRDELTKNQV | | | | | | |
| | | | SLWCLVKGFYPSDIAVEW | | | | | | |
| | | | ESNGQPENNYKTTPPVLDS | | | | | | |
| | | | DGSFFLYSKLTVDKSRWQ | | | | | | |
| | | | QGNVFSCSVMHEALHNH | | | | | | |
| | | | YTQKSLSLSPGK | | | | | | |
| EPI868 | HER3 | 222 | EVQLLESGGGLVQPGGSL | 223 | EVQLLESGGG | 224 | QSALTQPASVS | 225 | QSALTQP |
| | | | RLSCAASGFTFSHYVMAW | | LVQPGGSLRL | | GSPGQSITISCTG | | ASVSGSP |
| | | | VRQAPGKGLEWVSSISSSG | | SCAASGFTFS | | TSSDVGSYNVV | | GQSITISC |
| | | | GWTLYADSVKGRFTISRD | | HYVMAWVR | | SWYQQHPGKAP | | TGTSSDV |
| | | | NSKNTLYLQMNSLRAEDT | | QAPGKGLEW | | KLIIYEVSQRPSG | | GSYNVVS |
| | | | AVYYCTRGLKMATIFDY | | VSSISSSGGW | | VSNRFSGSKSGN | | WYQQHP |
| | | | WGQGTLVTVSSASTKGPS | | TLYADSVKG | | TASLTISGLQTE | | GKAPKLII |
| | | | VFPLAPCSRSTSESTAALG | | RFTISRDNSK | | DEADYYCCSYA | | YEVSQRP |
| | | | CLVKDYFPEPVTVSWNSG | | NTLYLQMNS | | GSSIFVIFGGGTK | | SGVSNRFS |
| | | | ALTSGVHTFPAVLQSSGLY | | LRAEDTAVY | | VTVLGQPKAAP | | GSKSGNT |
| | | | SLSSVVTVPSSNFGTQTYT | | YCTRGLKMA | | SVTLFPPSSEELQ | | ASLTISGL |
| | | | CNVDHKPSNTKVDKTVEP | | TIFDYWGQGT | | ANKATLVCLVS | | QTEDEAD |
| | | | KSCDKTHTCPPCPAPELLG | | LVTVSS | | DFYPGAVTVAW | | YYCCSYA |
| | | | GPSVFLFPPKPKDTLMISR | | | | KADGSPVKVGV | | GSSIFVIF |
| | | | TPEVTCVVVDVSHEDPEV | | | | ETTKPSKQSNNK | | GGGTKVTV |
| | | | KFNWYVDGVEVHNAKTK | | | | YAASSYLSLTPE | | L |
| | | | PREEQYNSTYRVVSVLTV | | | | QWKSHRSYSCR | | |
| | | | LHQDWLNGKEYKCKVSN | | | | VTHEGSTVEKT | | |
| | | | KALPAPIEKTISKAKGQPR | | | | VAPAECS | | |
| | | | EPQVYTLPPSRDELTKNQV | | | | | | |
| | | | SLWCLVKGFYPSDIAVEW | | | | | | |
| | | | ESNGQPENNYKTTPPVLDS | | | | | | |
| | | | DGSFFLYSKLTVDKSRWQ | | | | | | |
| | | | QGNVFSCSVMHEALHNH | | | | | | |
| | | | YTQKSLSLSPGK | | | | | | |
| EPI869 | CEA- | 226 | QVQLQESGPELKKPGETV | 227 | QVQLQESGPE | 228 | SIVMTQTPLSL | 229 | SIVMTQTP |
| | CAM5 | | KISCKASGYTFRNYGMNW | | LKKPGETVKI | | PVSLGDQASISC | | LSLPVSLG |
| | | | VKQAPGKGLKWMGWINT | | SCKASGYTFR | | QSSQSIVHSNGN | | DQASISCQ |
| | | | YTGEPTYADDFKGRFAFS | | NYGMNWVK | | TYLEWYLQKPG | | SSQSIVHS |
| | | | LETSASTAYLQINNVKNED | | QAPGKGLKW | | QSPNLLIYKVSN | | NGNTYLE |
| | | | TATYFCARKGWMDFNGS | | MGWINTYTG | | RFSGVPDRFSGS | | WYLQKPG |
| | | | SLDYWGQGTTVTVSSAST | | EPTYADDFKG | | GSGTDFTLKISR | | QSPNLLIY |
| | | | KGPSVFPLAPSSKSTSGGT | | RFAFSLETSAS | | VEAEDIGVYYCF | | KVSNRFS |
| | | | AALGCLVKDYFPEPVTVS | | TAYLQINNVK | | QGSHVPPTFGG | | GVPDRFS |
| | | | WNSGALTSGVHTFPAVLQ | | NEDTATYFCA | | GTKLEIKRTVAA | | GSGSGTD |
| | | | SSGLYSLSSVVTVPSSSLG | | RKGWMDFNG | | PSVFIFPPSDEQL | | FTLKISRV |
| | | | TQTYICNVNHKPSNTKVD | | SSLDYWGQG | | KSGTASVVCLL | | EAEDIGV |
| | | | KKVEPKSCDKTHTCPPCP | | TTVTVSS | | NNFYPREAKVQ | | YYCFQGS |
| | | | APELLGGPSVFLFPPKPKD | | | | WKVDNALQSG | | HVPPTFG |
| | | | TLMISRTPEVTCVVVDVSH | | | | NSQESVTEQDSK | | GGTKLEIK |
| | | | EDPEVKFNWYVDGVEVH | | | | DSTYSLSSTLTL | | |
| | | | NAKTKPREEQYNSTYRVV | | | | SKADYEKHKVY | | |
| | | | SVLTVLHQDWLNGKEYK | | | | ACEVTHQGLSSP | | |
| | | | CKVSNKALPAPIEKTISKA | | | | VTKSFNRGEC | | |
| | | | KGQPREPQVYTLPPSRDEL | | | | | | |
| | | | TKNQVSLWCLVKGFYPSD | | | | | | |
| | | | IAVEWESNGQPENNYKTT | | | | | | |
| | | | PPVLDSDGSFFLYSKLTVD | | | | | | |
| | | | KSRWQQGNVFSCSVMHE | | | | | | |
| | | | ALHNHYTQKSLSLSPGK | | | | | | |
| EPI870 | CEA- | 230 | EVRLVESGGGLVQGPGSL | 231 | EVRLVESGGG | 232 | DIQLTQSPAIM | 233 | DIQLTQSP |
| | CAM5 | | RLSCAASGFALTDYYMSW | | LVQGPGSLRL | | SASPGEKVTMT | | AIMSASPG |
| | | | VRQSPGKTLEWLGFIANK | | SCAASGFALT | | CSASSRVSYIHW | | EKVTMTC |
| | | | ANGHTTDYSPSVKGRFTIS | | DYYMSWVRQ | | YQQKSGTSPKR | | SASSRVSY |
| | | | RDNSQTILYLQMNTLRTE | | SPGKTLEWLG | | WIYGTSTLASGV | | IHWYQQK |
| | | | DSATYYCARDMGIRWNF | | FIANKANGHT | | PARFSGSGSGTS | | SGTSPKR |
| | | | DVWGQGTTVTVSSASTKG | | TDYSPSVKGR | | YSLTISSMEAED | | WIYGTST |
| | | | PSVFPLAPSSKSTSGGTAA | | FTISRDNSQTI | | AATYYCQQWSY | | LASGVPA |
| | | | LGCLVKDYFPEPVTVSWN | | LYLQMNTLR | | NPPTFGAGTKLE | | RFSGSGSG |
| | | | SGALTSGVHTFPAVLQSSG | | TEDSATYYCA | | LKRTVAAPSVFI | | TSYSLTIS |
| | | | LYSLSSVVTVPSSSLGTQT | | RDMGIRWNF | | FPPSDEQLKSGT | | SMEAEDA |
| | | | YICNVNHKPSNTKVDKKV | | DVWGQGTTV | | ASVVCLLNNFY | | ATYYCQQ |
| | | | EPKSCDKTHTCPPCPAPEL | | TVSS | | PREAKVQWKVD | | WSYNPPT |
| | | | LGGPSVFLFPPKPKDTLMI | | | | NALQSGNSQES | | FGAGTKL |
| | | | SRTPEVTCVVVDVSHEDP | | | | VTEQDSKDSTYS | | ELK |
| | | | EVKFNWYVDGVEVHNAK | | | | LSSTLTLSKADY | | |
| | | | TKPREEQYNSTYRVVSVL | | | | EKHKVYACEVT | | |
| | | | TVLHQDWLNGKEYKCKV | | | | HQGLSSPVTKSF | | |
| | | | SNKALPAPIEKTISKAKGQ | | | | NRGEC | | |
| | | | PREPQVYTLPPSRDELTKN | | | | | | |
| | | | QVSLWCLVKGFYPSDIAV | | | | | | |
| | | | EWESNGQPENNYKTTPPV | | | | | | |
| | | | LDSDGSFFLYSKLTVDKSR | | | | | | |
| | | | WQQGNVFSCSVMHEALH | | | | | | |
| | | | NHYTQKSLSLSPGK | | | | | | |
| EPI871 | CEA- | 234 | EVQLQESGPGLVKPSQTLS | 235 | EVQLQESGPG | 236 | EIVLTQSPATL | 237 | EIVLTQSP |
| | ACM5 | | LTCTVSDGSVSRGGYYLT | | LVKPSQTLSL | | SVSPGERATLSC | | ATLSVSPG |
| | | | WIRQHPGKGLEWIGYIYY | | TCTVSDGSVS | | RTSQSVRSNLA | | ERATLSC |
| | | | SGSTYFNPSLRSRVTMSVD | | RGGYYLTWIR | | WYQQKPGQAPR | | RTSQSVRS |
| | | | TSKNQFSLKLSSVTAADTA | | QHPGKGLEWI | | LLIYAASTRATG | | NLAWYQ |
| | | | VYYCARGIAVAPFDYWG | | GYIYYSGSTY | | IPARFSGSGSGT | | QKPGQAP |
| | | | QGTLVTVSSASTKGPSVFP | | FNPSLRSRVT | | EFTLTISSLQSED | | RLLIYAAS |
| | | | LAPSSKSTSGGTAALGCLV | | MSVDTSKNQ | | FAVYYCQQYTN | | TRATGIPA |
| | | | KDYFPEPVTVSWNSGALT | | FSLKLSSVTA | | WPFTFGPGTKV | | RFSGSGSG |
| | | | SGVHTFPAVLQSSGLYSLS | | ADTAVYYCA | | DIKRTVAAPSVF | | TEFTLTIS |
| | | | SVVTVPSSSLGTQTYICNV | | RGIAVAPFDY | | IFPPSDEQLKSGT | | SLQSEDFA |
| | | | NHKPSNTKVDKKVEPKSC | | WGQGTLVTV | | ASVVCLLNNFY | | VYYCQQY |
| | | | DKTHTCPPCPAPELLGGPS | | SS | | PREAKVQWKVD | | TNWPFTF |
| | | | VFLFPPKPKDTLMISRTPE | | | | NALQSGNSQES | | GPGTKVD |
| | | | VTCVVVDVSHEDPEVKFN | | | | VTEQDSKDSTYS | | IK |
| | | | WYVDGVEVHNAKTKPRE | | | | LSSTLTLSKADY | | |
| | | | EQYNSTYRVVSVLTVLHQ | | | | EKHKVYACEVT | | |
| | | | DWLNGKEYKCKVSNKAL | | | | HQGLSSPVTKSF | | |
| | | | PAPIEKTISKAKGQPREPQ | | | | NRGEC | | |
| | | | VYTLPPSRDELTKNQVSL | | | | | | |
| | | | WCLVKGFYPSDIAVEWES | | | | | | |
| | | | NGQPENNYKTTPPVLDSD | | | | | | |
| | | | GSFFLYSKLTVDKSRWQQ | | | | | | |
| | | | GNVFSCSVMHEALHNHYT | | | | | | |
| | | | QKSLSLSPGK | | | | | | |
| EPI872 | MUC1 | 238 | QMQLVQSEAELKKPGASV | 239 | QMQLVQSEA | 240 | QSVLTQPPSVS | 241 | QSVLTQPP |
| | | | KVSCKASGYSFTSHFMHW | | ELKKPGASVK | | VAPGKTARITCG | | SVSVAPG |
| | | | VRQAPGQGLEWMGWIDP | | VSCKASGYSF | | GNNIGSKSVHW | | KTARITCG |
| | | | VTGGTKYAQNFQGWVTM | | TSHFMHWVR | | YQQKPGQAPAL | | GNNIGSKS |
| | | | TRDTSIRTAYLELSRLRSD | | QAPGQGLEW | | VIYYGSNRPSGI | | VHWYQQ |
| | | | DTAMYYCAREARADRGQ | | MGWIDPVTG | | PERFSGSNSGNT | | KPGQAPA |
| | | | FDKWGQGTLVTVASASTK | | GTKYAQNFQ | | ATLTISRVEAGD | | LVIYYGS |
| | | | GPSVFPLAPSSKSTSGGTA | | GWVTMTRDT | | EADYYCQVWDS | | NRPSGIPE |
| | | | ALGCLVKDYFPEPVTVSW | | SIRTAYLELSR | | SSDWVFGGGTK | | RFSGSNSG |
| | | | NSGALTSGVHTFPAVLQSS | | LRSDDTAMY | | LTVLRTVAAPSV | | NTATLTIS |
| | | | GLYSLSSVVTVPSSSLGTQ | | YCAREARAD | | FIFPPSDEQLKSG | | RVEAGDE |
| | | | TYICNVNHKPSNTKVDKK | | RGQFDKWGQ | | TASVVCLLNNF | | ADYYCQV |
| | | | VEPKSCDKTHTCPPCPAPE | | GTLVTVAS | | YPREAKVQWKV | | WDSSSDW |
| | | | LLGGPSVFLFPPKPKDTLM | | | | DNALQSGNSQE | | VFGGGTK |
| | | | ISRTPEVTCVVVDVSHEDP | | | | SVTEQDSKDSTY | | LTVL |
| | | | EVKFNWYVDGVEVHNAK | | | | SLSSTLTLSKAD | | |
| | | | TKPREEQYNSTYRVVSVL | | | | YEKHKVYACEV | | |
| | | | TVLHQDWLNGKEYKCKV | | | | THQGLSSPVTKS | | |
| | | | SNKALPAPIEKTISKAKGQ | | | | FNRGEC | | |
| | | | PREPQVYTLPPSRDELTKN | | | | | | |
| | | | QVSLWCLVKGFYPSDIAV | | | | | | |
| | | | EWESNGQPENNYKTTPPV | | | | | | |
| | | | LDSDGSFFLYSKLTVDKSR | | | | | | |
| | | | WQQGNVFSCSVMHEALH | | | | | | |
| | | | NHYTQKSLSLSPGK | | | | | | |
| EPI873 | CD71 | 242 | EVQLVQSGAEVKKPGASV | 243 | EVQLVQSGAE | 244 | DIQMTQSPSSL | 245 | DIQMTQS |
| | | | KVSCKASGYTFTSYWMH | | VKKPGASVK | | SASVGDRVTITC | | PSSLSASV |
| | | | WVRQAPGQRLEWIGEINP | | VSCKASGYTF | | RASDNLYSNLA | | GDRVTITC |
| | | | TNGRTNYIEKFKSRATLTV | | TSYWMHWV | | WYQQKPGKSPK | | RASDNLY |
| | | | DKSASTAYMELSSLRSEDT | | RQAPGQRLE | | LLVYDATNLAD | | SNLAWYQ |
| | | | AVYYCARGTRAYHYWGQ | | WIGEINPTNG | | GVPSRFSGSGSG | | QKPGKSP |
| | | | GTMVTVSSASTKGPSVFPL | | RTNYIEKFKS | | TDYTLTISSLQPE | | KLLVYDA |
| | | | APSSKSTSGGTAALGCLV | | RATLTVDKSA | | DFATYYCQHFW | | TNLADGV |
| | | | KDYFPEPVTVSWNSGALT | | STAYMELSSL | | GTPLTFGQGTK | | PSRFSGSG |
| | | | SGVHTFPAVLQSSGLYSLS | | RSEDTAVYYC | | VEIKRTVAAPSV | | SGTDYTL |
| | | | SVVTVPSSSLGTQTYICNV | | ARGTRAYHY | | FIFPPSDEQLKSG | | TISSLQPE |
| | | | NHKPSNTKVDKKVEPKSC | | WGQGTMVTV | | TASVVCLLNNF | | DFATYYC |
| | | | DKTHTCPPCPAPELLGGPS | | SS | | YPREAKVQWKV | | QHFWGTP |
| | | | VFLFPPKPKDTLMISRTPE | | | | DNALQSGNSQE | | LTFGQGT |
| | | | VTCVVVDVSHEDPEVKFN | | | | SVTEQDSKDSTY | | KVEIK |
| | | | WYVDGVEVHNAKTKPRE | | | | SLSSTLTLSKAD | | |
| | | | EQYNSTYRVVSVLTVLHQ | | | | YEKHKVYACEV | | |
| | | | DWLNGKEYKCKVSNKAL | | | | THQGLSSPVTKS | | |
| | | | PAPIEKTISKAKGQPREPQ | | | | FNRGEC | | |
| | | | VYTLPPSRDELTKNQVSL | | | | | | |
| | | | WCLVKGFYPSDIAVEWES | | | | | | |
| | | | NGQPENNYKTTPPVLDSD | | | | | | |
| | | | GSFFLYSKLTVDKSRWQQ | | | | | | |
| | | | GNVFSCSVMHEALHNHYT | | | | | | |
| | | | QKSLSLSPGK | | | | | | |
| EPI874 | CD71 | 246 | EVQLVQSGAEVKKPGASV | 247 | EVQLVQSGAE | 248 | DIQMTQSPSSL | 249 | DIQMTQS |
| | | | KVSCKASGYTFTSYWMH | | VKKPGASVK | | SASVGDRVTITC | | PSSLSASV |
| | | | WVRQAPGQRLEWIGEIAP | | VSCKASGYTF | | RASDNLYSNLA | | GDRVTITC |
| | | | TNGRTNYIEKFKSRATLTV | | TSYWMHWV | | WYQQKPGKSPK | | RASDNLY |
| | | | DKSASTAYMELSSLRSEDT | | RQAPGQRLE | | LLVYDATNLAD | | SNLAWYQ |
| | | | AVYYCARGTRAYHYWGQ | | WIGEIAPTNG | | GVPSRFSGSGSG | | QKPGKSP |
| | | | GTMVTVSSASTKGPSVFPL | | RTNYIEKFKS | | TDYTLTISSLQPE | | KLLVYDA |
| | | | APSSKSTSGGTAALGCLV | | RATLTVDKSA | | DFATYYCQHFW | | TNLADGV |
| | | | KDYFPEPVTVSWNSGALT | | STAYMELSSL | | GTPLTFGQGTK | | PSRFSGSG |
| | | | SGVHTFPAVLQSSGLYSLS | | RSEDTAVYYC | | VEIKRTVAAPSV | | SGTDYTL |
| | | | SVVTVPSSSLGTQTYICNV | | ARGTRAYHY | | FIFPPSDEQLKSG | | TISSLQPE |
| | | | NHKPSNTKVDKKVEPKSC | | WGQGTMVTV | | TASVVCLLNNF | | DFATYYC |
| | | | DKTHTCPPCPAPELLGGPS | | SS | | YPREAKVQWKV | | QHFWGTP |
| | | | VFLFPPKPKDTLMISRTPE | | | | DNALQSGNSQE | | LTFGQGT |
| | | | VTCVVVDVSHEDPEVKFN | | | | SVTEQDSKDSTY | | KVEIK |
| | | | WYVDGVEVHNAKTKPRE | | | | SLSSTLTLSKAD | | |
| | | | EQYNSTYRVVSVLTVLHQ | | | | YEKHKVYACEV | | |
| | | | DWLNGKEYKCKVSNKAL | | | | THQGLSSPVTKS | | |
| | | | PAPIEKTISKAKGQPREPQ | | | | FNRGEC | | |
| | | | VYTLPPSRDELTKNQVSL | | | | | | |
| | | | WCLVKGFYPSDIAVEWES | | | | | | |
| | | | NGQPENNYKTTPPVLDSD | | | | | | |
| | | | GSFFLYSKLTVDKSRWQQ | | | | | | |
| | | | GNVFSCSVMHEALHNHYT | | | | | | |
| | | | QKSLSLSPGK | | | | | | |
| EPI875 | CD71 | 250 | EVQLVQSGAEVKKPGASV | 251 | EVQLVQSGAE | 252 | DIQMTQSPSSL | 253 | DIQMTQS |
| | | | KVSCKASGYTFTSYWMH | | VKKPGASVK | | SASVGDRVTITC | | PSSLSASV |
| | | | WVRQAPGQRLEWIGEINP | | VSCKASGYTF | | RASDNLYSNLA | | GDRVTITC |
| | | | ANGRTNYIEKFKSRATLTV | | TSYWMHWV | | WYQQKPGKSPK | | RASDNLY |
| | | | DKSASTAYMELSSLRSEDT | | RQAPGQRLE | | LLVYDATNLAD | | SNLAWYQ |
| | | | AVYYCARGTRAYHYWGQ | | WIGEINPANG | | GVPSRFSGSGSG | | QKPGKSP |
| | | | GTMVTVSSASTKGPSVFPL | | RTNYIEKFKS | | TDYTLTISSLQPE | | KLLVYDA |
| | | | APSSKSTSGGTAALGCLV | | RATLTVDKSA | | DFATYYCQHFW | | TNLADGV |
| | | | KDYFPEPVTVSWNSGALT | | STAYMELSSL | | GTPLTFGQGTK | | PSRFSGSG |
| | | | SGVHTFPAVLQSSGLYSLS | | RSEDTAVYYC | | VEIKRTVAAPSV | | SGTDYTL |
| | | | SVVTVPSSSLGTQTYICNV | | ARGTRAYHY | | FIFPPSDEQLKSG | | TISSLQPE |
| | | | NHKPSNTKVDKKVEPKSC | | WGQGTMVTV | | TASVVCLLNNF | | DFATYYC |
| | | | DKTHTCPPCPAPELLGGPS | | SS | | YPREAKVQWKV | | QHFWGTP |
| | | | VFLFPPKPKDTLMISRTPE | | | | DNALQSGNSQE | | LTFGQGT |
| | | | VTCVVVDVSHEDPEVKFN | | | | SVTEQDSKDSTY | | KVEIK |
| | | | WYVDGVEVHNAKTKPRE | | | | SLSSTLTLSKAD | | |
| | | | EQYNSTYRVVSVLTVLHQ | | | | YEKHKVYACEV | | |
| | | | DWLNGKEYKCKVSNKAL | | | | THQGLSSPVTKS | | |
| | | | PAPIEKTISKAKGQPREPQ | | | | FNRGEC | | |
| | | | VYTLPPSRDELTKNQVSL | | | | | | |
| | | | WCLVKGFYPSDIAVEWES | | | | | | |
| | | | NGQPENNYKTTPPVLDSD | | | | | | |
| | | | GSFFLYSKLTVDKSRWQQ | | | | | | |
| | | | GNVFSCSVMHEALHNHYT | | | | | | |
| | | | QKSLSLSPGK | | | | | | |
| EPI876 | CD71 | 254 | EVQLVQSGAEVKKPGASV | 255 | EVQLVQSGAE | 256 | DIQMTQSPSSL | 257 | DIQMTQS |
| | | | KVSCKASGYTFTSYWMH | | VKKPGASVK | | SASVGDRVTITC | | PSSLSASV |
| | | | WVRQAPGQRLEWIGEINP | | VSCKASGYTF | | RASDNLYSNLA | | GDRVTITC |
| | | | ANGRTNYIEKFKSRATLTV | | TSYWMHWV | | WYQQKPGKSPK | | RASDNLY |
| | | | DKSASTAYMELSSLRSEDT | | RQAPGQRLE | | LLVYDATNLAD | | SNLAWYQ |
| | | | AVYYCARGTRAYHYWGQ | | WIGEINPANG | | GVPSRFSGSGSG | | QKPGKSP |
| | | | GTMVTVSSASTKGPSVFPL | | RTNYIEKFKS | | TDYTLTISSLQPE | | KLLVYDA |
| | | | APSSKSTSGGTAALGCLV | | RATLTVDKSA | | DFATYYCQHFA | | TNLADGV |
| | | | KDYFPEPVTVSWNSGALT | | STAYMELSSL | | GTPLTFGQGTK | | PSRFSGSG |
| | | | SGVHTFPAVLQSSGLYSLS | | RSEDTAVYYC | | VEIKRTVAAPSV | | SGTDYTL |
| | | | SVVTVPSSSLGTQTYICNV | | ARGTRAYHY | | FIFPPSDEQLKSG | | TISSLQPE |
| | | | NHKPSNTKVDKKVEPKSC | | WGQGTMVTV | | TASVVCLLNNF | | DFATYYC |
| | | | DKTHTCPPCPAPELLGGPS | | SS | | YPREAKVQWKV | | QHFAGTP |
| | | | VFLFPPKPKDTLMISRTPE | | | | DNALQSGNSQE | | LTFGQGT |
| | | | VTCVVVDVSHEDPEVKFN | | | | SVTEQDSKDSTY | | KVEIK |
| | | | WYVDGVEVHNAKTKPRE | | | | SLSSTLTLSKAD | | |
| | | | EQYNSTYRVVSVLTVLHQ | | | | YEKHKVYACEV | | |
| | | | DWLNGKEYKCKVSNKAL | | | | THQGLSSPVTKS | | |
| | | | PAPIEKTISKAKGQPREPQ | | | | FNRGEC | | |
| | | | VYTLPPSRDELTKNQVSL | | | | | | |
| | | | WCLVKGFYPSDIAVEWES | | | | | | |
| | | | NGQPENNYKTTPPVLDSD | | | | | | |
| | | | GSFFLYSKLTVDKSRWQQ | | | | | | |
| | | | GNVFSCSVMHEALHNHYT | | | | | | |
| | | | QKSLSLSPGK | | | | | | |
| EPI1003 | CD276 | 258 | EVQLVESGGGLVQPGGSL | 259 | EVQLVESGGG | 260 | DIQLTQSPSFLS | 261 | DIQLTQSP |
| | | | RLSCAASGFTFSSFGMHW | | LVQPGGSLRL | | ASVGDRVTITCK | | SFLSASVG |
| | | | VRQAPGKGLEWVAYISSD | | SCAASGFTFS | | ASQNVDTNVA | | DRVTITCK |
| | | | SSAIYYADTVKGRFTISRD | | SFGMHWVRQ | | WYQQKPGKAPK | | ASQNVDT |
| | | | NAKNSLYLQMNSLRDEDT | | APGKGLEWV | | ALIYSASYRYSG | | NVAWYQ |
| | | | AVYYCGRGRENIYYGSRL | | AYISSDSSAIY | | VPSRFSGSGSGT | | QKPGKAP |
| | | | DYWGQGTTVTVSSASTKG | | YADTVKGRF | | DFTLTISSLQPED | | KALIYSAS |
| | | | PSVFPLAPSSKSTSGGTAA | | TISRDNAKNS | | FATYYCQQYNN | | YRYSGVP |
| | | | LGCLVKDYFPEPVTVSWN | | LYLQMNSLR | | YPFTFGQGTKLE | | SRFSGSGS |
| | | | SGALTSGVHTFPAVLQSSG | | DEDTAVYYC | | IKRTVAAPSVFIF | | GTDFTLTI |
| | | | LYSLSSVVTVPSSSLGTQT | | GRGRENIYYG | | PPSDEQLKSGTA | | SSLQPEDF |
| | | | YICNVNHKPSNTKVDKRV | | SRLDYWGQG | | SVVCLLNNFYPR | | ATYYCQQ |
| | | | EPKSCDKTHTCPPCPAPEL | | TTVTVSS | | EAKVQWKVDN | | YNNYPFT |
| | | | LGGPSVFLFPPKPKDTLMI | | | | ALQSGNSQESVT | | FGQGTKL |
| | | | SRTPEVTCVVVDVSHEDP | | | | EQDSKDSTYSLS | | EIK |
| | | | EVKFNWYVDGVEVHNAK | | | | STLTLSKADYEK | | |
| | | | TKPREEQYNSTYRVVSVL | | | | HKVYACEVTHQ | | |
| | | | TVLHQDWLNGKEYKCKV | | | | GLSSPVTKSFNR | | |
| | | | SNKALPAPIEKTISKAKGQ | | | | GEC | | |
| | | | PREPQVYTLPPSRDELTKN | | | | | | |
| | | | QVSLWCLVKGFYPSDIAV | | | | | | |
| | | | EWESNGQPENNYKTTPPV | | | | | | |
| | | | LDSDGSFFLYSKLTVDKSR | | | | | | |
| | | | WQQGNVFSCSVMHEALH | | | | | | |
| | | | NHYTQKSLSLSPGKGGSH | | | | | | |
| | | | HHHHH | | | | | | |
| EPI1004 | CEA- | 262 | EVQLVESGGGVVQPGRSL | 263 | EVQLVESGGG | 264 | DIQLTQSPSSLS | 265 | DIQLTQSP |
| | CAM5 | | RLSCSASGFDFTTYWMSW | | VVQPGRSLRL | | ASVGDRVTITCK | | SSLSASVG |
| | | | VRQAPGKGLEWIGEIHPDS | | SCSASGFDFT | | ASQDVGTSVAW | | DRVTITCK |
| | | | STINYAPSLKDRFTISRDN | | TYWMSWVR | | YQQKPGKAPKL | | ASQDVGT |
| | | | AKNTLFLQMDSLRPEDTG | | QAPGKGLEWI | | LIYWTSTRHTGV | | SVAWYQ |
| | | | VYFCASLYFGFPWFAYWG | | GEIHPDSSTIN | | PSRFSGSGSGTD | | QKPGKAP |
| | | | QGTPVTVSSASTKGPSVFP | | YAPSLKDRFT | | FTFTISSLQPEDI | | KLLIYWT |
| | | | LAPSSKSTSGGTAALGCLV | | ISRDNAKNTL | | ATYYCQQYSLY | | STRHTGV |
| | | | KDYFPEPVTVSWNSGALT | | FLQMDSLRPE | | RSFGQGTKVEIK | | PSRFSGSG |
| | | | SGVHTFPAVLQSSGLYSLS | | DTGVYFCASL | | RTVAAPSVFIFPP | | SGTDFTFT |
| | | | SVVTVPSSSLGTQTYICNV | | YFGFPWFAY | | SDEQLKSGTASV | | ISSLQPED |
| | | | NHKPSNTKVDKRVEPKSC | | WGQGTPVTV | | VCLLNNFYPRE | | IATYYCQQ |
| | | | DKTHTCPPCPAPELLGGPS | | SS | | AKVQWKVDNA | | YSLYRSF |
| | | | VFLFPPKPKDTLMISRTPE | | | | LQSGNSQESVTE | | GQGTKVE |
| | | | VTCVVVDVSHEDPEVKFN | | | | QDSKDSTYSLSS | | IK |
| | | | WYVDGVEVHNAKTKPRE | | | | TLTLSKADYEK | | |
| | | | EQYNSTYRVVSVLTVLHQ | | | | HKVYACEVTHQ | | |
| | | | DWLNGKEYKCKVSNKAL | | | | GLSSPVTKSFNR | | |
| | | | PAPIEKTISKAKGQPREPQ | | | | GEC | | |
| | | | VYTLPPSRDELTKNQVSL | | | | | | |
| | | | WCLVKGFYPSDIAVEWES | | | | | | |
| | | | NGQPENNYKTTPPVLDSD | | | | | | |
| | | | GSFFLYSKLTVDKSRWQQ | | | | | | |
| | | | GNVFSCSVMHEALHNHYT | | | | | | |
| | | | QKSLSLSPGK | | | | | | |
| EPI1005 | LGR5 | 266 | EVQLVQSGSKLKKPGASV | 267 | EVQLVQSGSK | 268 | DIQMTQSPSSL | 269 | DIQMTQS |
| | | | KVSCKASGYTFTSYTMNW | | LKKPGASVK | | SASVGDRVTITC | | PSSLSASV |
| | | | VRQAPGQGLEWMGWINT | | VSCKASGYTF | | RASQSISSYLNW | | GDRVTITC |
| | | | DTGDPTYAQGFTGRFVFS | | TSYTMNWVR | | YQQKPGKAPKL | | RASQSISS |
| | | | LDTSVSTAFLQINSLKAED | | QAPGQGLEW | | LIYAASSLQSGV | | YLNWYQ |
| | | | TAVYYCARGDCDSTSCYR | | MGWINTDTG | | PSRFSGSGSGTD | | QKPGKAP |
| | | | YSYGYEDYWGQGTLVTV | | DPTYAQGFTG | | FTLTISSLQPEDF | | KLLIYAAS |
| | | | SSASTKGPSVFPLAPSSKST | | RFVFSLDTSV | | ATYYCQQSYST | | SLQSGVPS |
| | | | SGGTAALGCLVKDYFPEP | | STAFLQINSLK | | PPTFGQGTKVEI | | RFSGSGSG |
| | | | VTVSWNSGALTSGVHTFP | | AEDTAVYYC | | KRTVAAPSVFIF | | TDFTLTIS |
| | | | AVLQSSGLYSLSSVVTVPS | | ARGDCDSTSC | | PPSDEQLKSGTA | | SLQPEDFA |
| | | | SSLGTQTYICNVNHKPSNT | | YRYSYGYED | | SVVCLLNNFYPR | | TYYCQQS |
| | | | KVDKKVEPKSCDKTHTCP | | YWGQGTLVT | | EAKVQWKVDN | | YSTPPTFG |
| | | | PCPAPELLGGPSVFLFPPKP | | VSS | | ALQSGNSQESVT | | QGTKVEI |
| | | | KDTLMISRTPEVTCVVVD | | | | EQDSKDSTYSLS | | K |
| | | | VSHEDPEVKFNWYVDGV | | | | STLTLSKADYEK | | |
| | | | EVHNAKTKPREEQYNSTY | | | | HKVYACEVTHQ | | |
| | | | RVVSVLTVLHQDWLNGK | | | | GLSSPVTKSFNR | | |
| | | | EYKCKVSNKALPAPIEKTI | | | | GEC | | |
| | | | SKAKGQPREPQVYTLPPSR | | | | | | |
| | | | DELTKNQVSLWCLVKGFY | | | | | | |
| | | | PSDIAVEWESNGQPENNY | | | | | | |
| | | | KTTPPVLDSDGSFFLYSKL | | | | | | |
| | | | TVDKSRWQQGNVFSCSV | | | | | | |
| | | | MHEALHNHYTQKSLSLSP | | | | | | |
| | | | GK | | | | | | |
| EPI1006 | CEA- | 270 | EVQLQESGPGLVKPGGSL | 271 | EVQLQESGPG | 272 | DIQMTQSPASL | 273 | DIQMTQS |
| | CAM5 | | SLSCAASGFVFSSYDMSW | | LVKPGGSLSL | | SASVGDRVTITC | | PASLSASV |
| | | | VRQTPERGLEWVAYISSG | | SCAASGFVFS | | RASENIFSYLAW | | GDRVTITC |
| | | | GGITYAPSTVKGRFTVSRD | | SYDMSWVRQ | | YQQKPGKSPKL | | RASENIFS |
| | | | NAKNTLYLQMNSLTSEDT | | TPERGLEWV | | LVYNTRTLAEG | | YLAWYQ |
| | | | AVYYCAAHYFGSSGPFAY | | AYISSGGGITY | | VPSRFSGSGSGT | | QKPGKSP |
| | | | WGQGTLVTVSSASTKGPS | | APSTVKGRFT | | DFSLTISSLQPED | | KLLVYNT |
| | | | VFPLAPSSKSTSGGTAALG | | VSRDNAKNT | | FATYYCQHHYG | | RTLAEGV |
| | | | CLVKDYFPEPVTVSWNSG | | LYLQMNSLTS | | TPFTFGSGTKLEI | | PSRFSGSG |
| | | | ALTSGVHTFPAVLQSSGLY | | EDTAVYYCA | | KRTVAAPSVFIF | | SGTDFSLT |
| | | | SLSSVVTVPSSSLGTQTYIC | | AHYFGSSGPF | | PPSDEQLKSGTA | | ISSLQPED |
| | | | NVNHKPSNTKVDKKVEPK | | AYWGQGTLV | | SVVCLLNNFYPR | | FATYYCQ |
| | | | SCDKTHTCPPCPAPELLGG | | TVSS | | EAKVQWKVDN | | HHYGTPF |
| | | | PSVFLFPPKPKDTLMISRTP | | | | ALQSGNSQESVT | | TFGSGTK |
| | | | EVTCVVVDVSHEDPEVKF | | | | EQDSKDSTYSLS | | LEIK |
| | | | NWYVDGVEVHNAKTKPR | | | | STLTLSKADYEK | | |
| | | | EEQYNSTYRVVSVLTVLH | | | | HKVYACEVTHQ | | |
| | | | QDWLNGKEYKCKVSNKA | | | | GLSSPVTKSFNR | | |
| | | | LPAPIEKTISKAKGQPREPQ | | | | GEC | | |
| | | | VYTLPPSRDELTKNQVSL | | | | | | |
| | | | WCLVKGFYPSDIAVEWES | | | | | | |
| | | | NGQPENNYKTTPPVLDSD | | | | | | |
| | | | GSFFLYSKLTVDKSRWQQ | | | | | | |
| | | | GNVFSCSVMHEALHNHYT | | | | | | |
| | | | QKSLSLSPGK | | | | | | |
| EPI1007 | RNF43 | 274 | QVQLKESGPGLVQPSQTL | 275 | QVQLKESGPG | 276 | DTVLTQSPALA | 277 | DTVLTQS |
| | | | SLTCTVSGFSLTTYSVHW | | LVQPSQTLSL | | VSPGERVTISCR | | PALAVSP |
| | | | VRQHSGKNLEWMGRMW | | TCTVSGFSLT | | ASESVSKLMHW | | GERVTISC |
| | | | TAGDTSYNSAFTSRLNIFR | | TYSVHWVRQ | | YQQRPGQQPQL | | RASESVS |
| | | | DTSKSQVFLKMNSLQTED | | HSGKNLEWM | | LIYLTSHLASGV | | KLMHWY |
| | | | TGTYYCARSSYTSGYPFDS | | GRMWTAGDT | | PARFSGSGSGTD | | QQRPGQQ |
| | | | WGQGVMVTVSSASTKGPS | | SYNSAFTSRL | | FTLTIDPVEADD | | PQLLIYLT |
| | | | VFPLAPSSKSTSGGTAALG | | NIFRDTSKSQ | | TATYYCQQSRN | | SHLASGV |
| | | | CLVKDYFPEPVTVSWNSG | | VFLKMNSLQT | | DPTFGAGTKLEL | | PARFSGSG |
| | | | ALTSGVHTFPAVLQSSGLY | | EDTGTYYCA | | KRTVAAPSVFIF | | SGTDFTLT |
| | | | SLSSVVTVPSSSLGTQTYIC | | RSSYTSGYPF | | PPSDEQLKSGTA | | IDPVEAD |
| | | | NVNHKPSNTKVDKKVEPK | | DSWGQGVMV | | SVVCLLNNFYPR | | DTATYYC |
| | | | SCDKTHTCPPCPAPELLGG | | TVSS | | EAKVQWKVDN | | QQSRNDP |
| | | | PSVFLFPPKPKDTLMISRTP | | | | ALQSGNSQESVT | | TFGAGTK |
| | | | EVTCVVVDVSHEDPEVKF | | | | EQDSKDSTYSLS | | LELK |
| | | | NWYVDGVEVHNAKTKPR | | | | STLTLSKADYEK | | |
| | | | EEQYNSTYRVVSVLTVLH | | | | HKVYACEVTHQ | | |
| | | | QDWLNGKEYKCKVSNKA | | | | GLSSPVTKSFNR | | |
| | | | LPAPIEKTISKAKGQPREPQ | | | | GEC | | |
| | | | VYTLPPSRDELTKNQVSL | | | | | | |
| | | | WCLVKGFYPSDIAVEWES | | | | | | |
| | | | NGQPENNYKTTPPVLDSD | | | | | | |
| | | | GSFFLYSKLTVDKSRWQQ | | | | | | |
| | | | GNVFSCSVMHEALHNHYT | | | | | | |
| | | | QKSLSLSPGKGGSHHHHH | | | | | | |
| | | | H | | | | | | |
| EPI1008 | RNF43 | 278 | EVQLVESGGGLVQPGGSL | 279 | EVQLVESGGG | 280 | DIQMTQSPSSL | 281 | DIQMTQS |
| | | | RLSCVVSGFTFSYYDMHW | | LVQPGGSLRL | | SASVGDRVTITC | | PSSLSASV |
| | | | VRQVTGKGLEWVSAIGTA | | SCVVSGFTFS | | RASQSISSYLNW | | GDRVTITC |
| | | | GATYYPGSVKGRFTISREN | | YYDMHWVR | | YQQKPGKAPKL | | RASQSISS |
| | | | AKNSLYLQMNSLRAGDTA | | QVTGKGLEW | | LIYAASSLQSGV | | YLNWYQ |
| | | | VYYCARDRGYSGYDAYY | | VSAIGTAGAT | | PSRFSGSGSGTD | | QKPGKAP |
| | | | FDFWGQGTLVTVSSASTK | | YYPGSVKGRF | | FTLTISSLQPEDF | | KLLIYAAS |
| | | | GPSVFPLAPSSKSTSGGTA | | TISRENAKNS | | ATYYCQQSYST | | SLQSGVPS |
| | | | ALGCLVKDYFPEPVTVSW | | LYLQMNSLR | | PPTFGQGTKVEI | | RFSGSGSG |
| | | | NSGALTSGVHTFPAVLQSS | | AGDTAVYYC | | KRTVAAPSVFIF | | TDFTLTIS |
| | | | GLYSLSSVVTVPSSSLGTQ | | ARDRGYSGY | | PPSDEQLKSGTA | | SLQPEDFA |
| | | | TYICNVNHKPSNTKVDKK | | DAYYFDFWG | | SVVCLLNNFYPR | | TYYCQQS |
| | | | VEPKSCDKTHTCPPCPAPE | | QGTLVTVSS | | EAKVQWKVDN | | YSTPPTFG |
| | | | LLGGPSVFLFPPKPKDTLM | | | | ALQSGNSQESVT | | QGTKVEI |
| | | | ISRTPEVTCVVVDVSHEDP | | | | EQDSKDSTYSLS | | K |
| | | | EVKFNWYVDGVEVHNAK | | | | STLTLSKADYEK | | |
| | | | TKPREEQYNSTYRVVSVL | | | | HKVYACEVTHQ | | |
| | | | TVLHQDWLNGKEYKCKV | | | | GLSSPVTKSFNR | | |
| | | | SNKALPAPIEKTISKAKGQ | | | | GEC | | |
| | | | PREPQVYTLPPSRDELTKN | | | | | | |
| | | | QVSLWCLVKGFYPSDIAV | | | | | | |
| | | | EWESNGQPENNYKTTPPV | | | | | | |
| | | | LDSDGSFFLYSKLTVDKSR | | | | | | |
| | | | WQQGNVFSCSVMHEALH | | | | | | |
| | | | NHYTQKSLSLSPGKGGSH | | | | | | |
| | | | HHHHH | | | | | | |
| EPI1009 | RNF43 | 282 | EVQLVQSGGGLVQPGGSL | 283 | EVQLVQSGG | 284 | DIQMTQSPSSL | 285 | DIQMTQS |
| | | | RLSCAASGFTFSYYDMHW | | GLVQPGGSLR | | SASVGDRVTITC | | PSSLSASV |
| | | | VRQVTGKGLEWVSTIGAT | | LSCAASGFTF | | RASQSISSYLNW | | GDRVTITC |
| | | | GDTYYSDSVKGRFTISRQN | | SYYDMHWVR | | YQQKPGKAPKL | | RASQSISS |
| | | | AKNSLYLQINSLRAGDTA | | QVTGKGLEW | | LIYAASSLQSGV | | YLNWYQ |
| | | | VYYCVRDRGYIGYDSYYF | | VSTIGATGDT | | PSRFSGSGSGTD | | QKPGKAP |
| | | | DNWGQGTLVTVSSASTKG | | YYSDSVKGRF | | FTLTISSLQPEDF | | KLLIYAAS |
| | | | PSVFPLAPSSKSTSGGTAA | | TISRQNAKNS | | ATYYCQQSYST | | SLQSGVPS |
| | | | LGCLVKDYFPEPVTVSWN | | LYLQINSLRA | | PPTFGQGTKVEI | | RFSGSGSG |
| | | | SGALTSGVHTFPAVLQSSG | | GDTAVYYCV | | KRTVAAPSVFIF | | TDFTLTIS |
| | | | LYSLSSVVTVPSSSLGTQT | | RDRGYIGYDS | | PPSDEQLKSGTA | | SLQPEDFA |
| | | | YICNVNHKPSNTKVDKKV | | YYFDNWGQG | | SVVCLLNNFYPR | | TYYCQQS |
| | | | EPKSCDKTHTCPPCPAPEL | | TLVTVSS | | EAKVQWKVDN | | YSTPPTFG |
| | | | LGGPSVFLFPPKPKDTLMI | | | | ALQSGNSQESVT | | QGTKVEI |
| | | | SRTPEVTCVVVDVSHEDP | | | | EQDSKDSTYSLS | | K |
| | | | EVKFNWYVDGVEVHNAK | | | | STLTLSKADYEK | | |
| | | | TKPREEQYNSTYRVVSVL | | | | HKVYACEVTHQ | | |
| | | | TVLHQDWLNGKEYKCKV | | | | GLSSPVTKSFNR | | |
| | | | SNKALPAPIEKTISKAKGQ | | | | GEC | | |
| | | | PREPQVYTLPPSRDELTKN | | | | | | |
| | | | QVSLWCLVKGFYPSDIAV | | | | | | |
| | | | EWESNGQPENNYKTTPPV | | | | | | |
| | | | LDSDGSFFLYSKLTVDKSR | | | | | | |
| | | | WQQGNVFSCSVMHEALH | | | | | | |
| | | | NHYTQKSLSLSPGKGGSH | | | | | | |
| | | | HHHHH | | | | | | |
| EPI1010 | ITGB6 | 286 | QVQLVQSGAEVKKPGAS | 287 | QVQLVQSGA | 288 | DVVMTQSPLS | 289 | DVVMTQS |
| | | | VKVSCKASGYSFSGYFMN | | EVKKPGASV | | LPVTLGQPASIS | | PLSLPVTL |
| | | | WVRQAPGQGLEWMGLIN | | KVSCKASGYS | | CKSSQSLLDSDG | | GQPASISC |
| | | | PYNGDSFYNQKFKGRVTM | | FSGYFMNWV | | KTYLNWLFQRP | | KSSQSLLD |
| | | | TRQTSTSTVYMELSSLRSE | | RQAPGQGLE | | GQSPRRLIYLVS | | SDGKTYL |
| | | | DTAVYYCVRGLRRDFDY | | WMGLINPYN | | ELDSGVPDRFSG | | NWLFQRP |
| | | | WGQGTLVTVSSASTKGPS | | GDSFYNQKFK | | SGSGTDFTLKIS | | GQSPRRLI |
| | | | VFPLAPSSKSTSGGTAALG | | GRVTMTRQT | | RVEAEDVGVYY | | YLVSELD |
| | | | CLVKDYFPEPVTVSWNSG | | STSTVYMELS | | CWQGTHFPRTF | | SGVPDRFS |
| | | | ALTSGVHTFPAVLQSSGLY | | SLRSEDTAVY | | GGGTKLEIKRTV | | GSGSGTD |
| | | | SLSSVVTVPSSSLGTQTYIC | | YCVRGLRRDF | | AAPSVFIFPPSDE | | FTLKISRV |
| | | | NVNHKPSNTKVDKKVEPK | | DYWGQGTLV | | QLKSGTASVVC | | EAEDVGV |
| | | | SCDKTHTCPPCPAPELLGG | | TVSS | | LLNNFYPREAK | | YYCWQG |
| | | | PSVFLFPPKPKDTLMISRTP | | | | VQWKVDNALQ | | THFPRTFG |
| | | | EVTCVVVDVSHEDPEVKF | | | | SGNSQESVTEQD | | GGTKLEIK |
| | | | NWYVDGVEVHNAKTKPR | | | | SKDSTYSLSSTL | | |
| | | | EEQYNSTYRVVSVLTVLH | | | | TLSKADYEKHK | | |
| | | | QDWLNGKEYKCKVSNKA | | | | VYACEVTHQGL | | |
| | | | LPAPIEKTISKAKGQPREPQ | | | | SSPVTKSFNRGE | | |
| | | | VYTLPPSRDELTKNQVSL | | | | C | | |
| | | | WCLVKGFYPSDIAVEWES | | | | | | |
| | | | NGQPENNYKTTPPVLDSD | | | | | | |
| | | | GSFFLYSKLTVDKSRWQQ | | | | | | |
| | | | GNVFSCSVMHEALHNHYT | | | | | | |
| | | | QKSLSLSPGKGGSHHHHH | | | | | | |
| | | | H | | | | | | |
| EPI1011 | HER3 | 290 | QVQLQQWGAGLLKPSETL | 291 | QVQLQQWGA | 292 | DIEMTQSPDSL | 293 | DIEMTQSP |
| | | | SLTCAVYGGSFSGYYWSW | | GLLKPSETLS | | AVSLGERATINC | | DSLAVSL |
| | | | IRQPPGKGLEWIGEINHSG | | LTCAVYGGSF | | RSSQSVLYSSSN | | GERATINC |
| | | | STNYNPSLKSRVTISVETS | | SGYYWSWIR | | RNYLAWYQQNP | | RSSQSVL |
| | | | KNQFSLKLSSVTAADTAV | | QPPGKGLEWI | | GQPPKLLIYWAS | | YSSSNRN |
| | | | YYCARDKWTWYFDLWG | | GEINHSGSTN | | TRESGVPDRFSG | | YLAWYQ |
| | | | RGTLVTVSSASTKGPSVFP | | YNPSLKSRVT | | SGSGTDFTLTISS | | QNPGQPP |
| | | | LAPSSKSTSGGTAALGCLV | | ISVETSKNQFS | | LQAEDVAVYYC | | KLLIYWA |
| | | | KDYFPEPVTVSWNSGALT | | LKLSSVTAAD | | QQYYSTPRTFG | | STRESGVP |
| | | | SGVHTFPAVLQSSGLYSLS | | TAVYYCARD | | QGTKVEIKRTV | | DRFSGSGS |
| | | | SVVTVPSSSLGTQTYICNV | | KWTWYFDL | | AAPSVFIFPPSDE | | GTDFTLTI |
| | | | NHKPSNTKVDKRVEPKSC | | WGRGTLVTV | | QLKSGTASVVC | | SSLQAED |
| | | | DKTHTCPPCPAPELLGGPS | | SS | | LLNNFYPREAK | | VAVYYCQ |
| | | | VFLFPPKPKDTLMISRTPE | | | | VQWKVDNALQ | | QYYSTPR |
| | | | VTCVVVDVSHEDPEVKFN | | | | SGNSQESVTEQD | | TFGQGTK |
| | | | WYVDGVEVHNAKTKPRE | | | | SKDSTYSLSSTL | | VEIK |
| | | | EQYNSTYRVVSVLTVLHQ | | | | TLSKADYEKHK | | |
| | | | DWLNGKEYKCKVSNKAL | | | | VYACEVTHQGL | | |
| | | | PAPIEKTISKAKGQPREPQ | | | | SSPVTKSFNRGE | | |
| | | | VYTLPPSRDELTKNQVSL | | | | C | | |
| | | | WCLVKGFYPSDIAVEWES | | | | | | |
| | | | NGQPENNYKTTPPVLDSD | | | | | | |
| | | | GSFFLYSKLTVDKSRWQQ | | | | | | |
| | | | GNVFSCSVMHEALHNHYT | | | | | | |
| | | | QKSLSLSPGKGGSHHHHH | | | | | | |
| | | | H | | | | | | |
| EPI1012 | CD142 | 294 | EVQLLESGGGLVQPGGSL | 295 | EVQLLESGGG | 296 | DIQMTQSPPSL | 297 | DIQMTQS |
| | | | RLSCAASGFTFSNYAMSW | | LVQPGGSLRL | | SASAGDRVTITC | | PPSLSASA |
| | | | VRQAPGKGLEWVSSISGS | | SCAASGFTFS | | RASQGISSRLAW | | GDRVTITC |
| | | | GDYTYYTDSVKGRFTISR | | NYAMSWVRQ | | YQQKPEKAPKS | | RASQGISS |
| | | | DNSKNTLYLQMNSLRAED | | APGKGLEWV | | LIYAASSLQSGV | | RLAWYQ |
| | | | TAVYYCARSPWGYYLDS | | SSISGSGDYT | | PSRFSGSGSGTD | | QKPEKAP |
| | | | WGQGTLVTVSSASTKGPS | | YYTDSVKGRF | | FTLTISSLQPEDF | | KSLIYAAS |
| | | | VFPLAPSSKSTSGGTAALG | | TISRDNSKNT | | ATYYCQQYNSY | | SLQSGVPS |
| | | | CLVKDYFPEPVTVSWNSG | | LYLQMNSLR | | PYTFGQGTKLEI | | RFSGSGSG |
| | | | ALTSGVHTFPAVLQSSGLY | | AEDTAVYYC | | KRTVAAPSVFIF | | TDFTLTIS |
| | | | SLSSVVTVPSSSLGTQTYIC | | ARSPWGYYL | | PPSDEQLKSGTA | | SLQPEDFA |
| | | | NVNHKPSNTKVDKRVEPK | | DSWGQGTLV | | SVVCLLNNFYPR | | TYYCQQY |
| | | | SCDKTHTCPPCPAPELLGG | | TVSS | | EAKVQWKVDN | | NSYPYTF |
| | | | PSVFLFPPKPKDTLMISRTP | | | | ALQSGNSQESVT | | GQGTKLEI |
| | | | EVTCVVVDVSHEDPEVKF | | | | EQDSKDSTYSLS | | K |
| | | | NWYVDGVEVHNAKTKPR | | | | STLTLSKADYEK | | |
| | | | EEQYNSTYRVVSVLTVLH | | | | HKVYACEVTHQ | | |
| | | | QDWLNGKEYKCKVSNKA | | | | GLSSPVTKSFNR | | |
| | | | LPAPIEKTISKAKGQPREPQ | | | | GEC | | |
| | | | VYTLPPSRDELTKNQVSL | | | | | | |
| | | | WCLVKGFYPSDIAVEWES | | | | | | |
| | | | NGQPENNYKTTPPVLDSD | | | | | | |
| | | | GSFFLYSKLTVDKSRWQQ | | | | | | |
| | | | GNVFSCSVMHEALHNHYT | | | | | | |
| | | | QKSLSLSPGK | | | | | | |
| EPI1013 | RNF130 | 298 | QVQLQESGGGLVQAGGSL | 299 | QVQLQESGG | | | | |
| | | | RLSCAASGYISGYYVMGW | | GLVQAGGSL | | | | |
| | | | YRQAPGKEREFVASISYGA | | RLSCAASGYI | | | | |
| | | | STYYADSVKGRFTISRDNA | | SGYYVMGWY | | | | |
| | | | KNTVYLQMNSLKPEDTAV | | RQAPGKEREF | | | | |
| | | | YYCAVDFDSNYAHTYWG | | VASISYGAST | | | | |
| | | | QGTQVTVSSGGGGSDKTH | | YYADSVKGR | | | | |
| | | | TCPPCPAPELLGGPSVFLFP | | FTISRDNAKN | | | | |
| | | | PKPKDTLMISRTPEVTCVV | | TVYLQMNSL | | | | |
| | | | VDVSHEDPEVKFNWYVD | | KPEDTAVYY | | | | |
| | | | GVEVHNAKTKPREEQYNS | | CAVDFDSNY | | | | |
| | | | TYRVVSVLTVLHQDWLN | | AHTYWGQGT | | | | |
| | | | GKEYKCKVSNKALPAPIE | | QVTVSS | | | | |
| | | | KTISKAKGQPREPQVYTLP | | | | | | |
| | | | PSRDELTKNQVSLWCLVK | | | | | | |
| | | | GFYPSDIAVEWESNGQPE | | | | | | |
| | | | NNYKTTPPVLDSDGSFFLY | | | | | | |
| | | | SKLTVDKSRWQQGNVFSC | | | | | | |
| | | | SVMHEALHNHYTQKSLSL | | | | | | |
| | | | SPGKGGSHHHHHH | | | | | | |
| EPI1014 | RNF130 | 300 | QVQLQESGGGLVQAGGSL | 301 | QVQLQESGG | | | | |
| | | | RLSCAASGTISFIGYMGWY | | GLVQAGGSL | | | | |
| | | | RQAPGKERELVASIASGTS | | RLSCAASGTIS | | | | |
| | | | TYYADSVKGRFTISRDNA | | FIGYMGWYR | | | | |
| | | | KNTVYLQMNSLKPEDTAV | | QAPGKERELV | | | | |
| | | | YYCAATQYIQDVHRYWG | | ASIASGTSTY | | | | |
| | | | QGTQVTVSSGGGGSDKTH | | YADSVKGRFT | | | | |
| | | | TCPPCPAPELLGGPSVFLFP | | ISRDNAKNTV | | | | |
| | | | PKPKDTLMISRTPEVTCVV | | YLQMNSLKPE | | | | |
| | | | VDVSHEDPEVKFNWYVD | | DTAVYYCAA | | | | |
| | | | GVEVHNAKTKPREEQYNS | | TQYIQDVHRY | | | | |
| | | | TYRVVSVLTVLHQDWLN | | WGQGTQVTV | | | | |
| | | | GKEYKCKVSNKALPAPIE | | SS | | | | |
| | | | KTISKAKGQPREPQVYTLP | | | | | | |
| | | | PSRDELTKNQVSLWCLVK | | | | | | |
| | | | GFYPSDIAVEWESNGQPE | | | | | | |
| | | | NNYKTTPPVLDSDGSFFLY | | | | | | |
| | | | SKLTVDKSRWQQGNVFSC | | | | | | |
| | | | SVMHEALHNHYTQKSLSL | | | | | | |
| | | | SPGKGGSHHHHHH | | | | | | |
| EPI1015 | CD71 | 302 | QVQLVQSGAEVKKPGAS | 303 | QVQLVQSGA | 304 | DIQMTQSPSSL | 305 | DIQMTQS |
| | | | VKMSCKASGYTFTSYWM | | EVKKPGASV | | SASVGDRVTITC | | PSSLSASV |
| | | | HWVRQAPGQGLEWIGAIY | | KMSCKASGY | | SASSSVYYMYW | | GDRVTITC |
| | | | PGNSETGYAQKFQGRATL | | TFTSYWMHW | | FQQKPGKAPKL | | SASSSVY |
| | | | TADTSTSTAYMELSSLRSE | | VRQAPGQGL | | WIYSTSNLASGV | | YMYWFQ |
| | | | DTAVYYCTRENWDPGFAF | | EWIGAIYPGN | | PSRFSGSGSGTD | | QKPGKAP |
| | | | WGQGTLITVSSASTKGPSV | | SETGYAQKFQ | | YTLTISSMQPED | | KLWIYSTS |
| | | | FPLAPSSKSTSGGTAALGC | | GRATLTADTS | | FATYYCQQRRN | | NLASGVP |
| | | | LVKDYFPEPVTVSWNSGA | | TSTAYMELSS | | YPYTFGQGTKL | | SRFSGSGS |
| | | | LTSGVHTFPAVLQSSGLYS | | LRSEDTAVYY | | EIKRTVAAPSVFI | | GTDYTLTI |
| | | | LSSVVTVPSSSLGTQTYIC | | CTRENWDPG | | FPPSDEQLKSGT | | SSMQPED |
| | | | NVNHKPSNTKVDKKVEPK | | FAFWGQGTLI | | ASVVCLLNNFY | | FATYYCQ |
| | | | SCDKTHTCPPCPAPELLGG | | TVSS | | PREAKVQWKVD | | QRRNYPY |
| | | | PSVFLFPPKPKDTLMISRTP | | | | NALQSGNSQES | | TFGQGTK |
| | | | EVTCVVVDVSHEDPEVKF | | | | VTEQDSKDSTYS | | LEIK |
| | | | NWYVDGVEVHNAKTKPR | | | | LSSTLTLSKADY | | |
| | | | EEQYNSTYRVVSVLTVLH | | | | EKHKVYACEVT | | |
| | | | QDWLNGKEYKCKVSNKA | | | | HQGLSSPVTKSF | | |
| | | | LPAPIEKTISKAKGQPREPQ | | | | NRGEC | | |
| | | | VYTLPPSRDELTKNQVSL | | | | | | |
| | | | WCLVKGFYPSDIAVEWES | | | | | | |
| | | | NGQPENNYKTTPPVLDSD | | | | | | |
| | | | GSFFLYSKLTVDKSRWQQ | | | | | | |
| | | | GNVFSCSVMHEALHNHYT | | | | | | |
| | | | QKSLSLSPGK | | | | | | |
| EPI1022 | HER3 | 306 | EVQLVESGGGLVQPGGSL | 307 | EVQLVESGGG | 308 | DIQMTQSPSSL | 309 | DIQMTQS |
| | | | RLSCAASGFTLSGDWIHW | | LVQPGGSLRL | | SASVGDRVTITC | | PSSLSASV |
| | | | VRQAPGKGLEWVGEISAA | | SCAASGFTLS | | RASQNIATDVA | | GDRVTITC |
| | | | GGYTDYADSVKGRFTISA | | GDWIHWVRQ | | WYQQKPGKAPK | | RASQNIAT |
| | | | DTSKNTAYLQMNSLRAED | | APGKGLEWV | | LLIYSASFLYSG | | DVAWYQ |
| | | | TAVYYCARESRVSFEAAM | | GEISAAGGYT | | VPSRFSGSGSGT | | QKPGKAP |
| | | | DYWGQGTLVTVSSASTKG | | DYADSVKGR | | DFTLTISSLQPED | | KLLIYSAS |
| | | | PSVFPLAPSSKSTSGGTAA | | FTISADTSKNT | | FATYYCQQSEPE | | FLYSGVPS |
| | | | LGCLVKDYFPEPVTVSWN | | AYLQMNSLR | | PYTFGQGTKVEI | | RFSGSGSG |
| | | | SGALTSGVHTFPAVLQSSG | | AEDTAVYYC | | KRTVAAPSVFIF | | TDFTLTIS |
| | | | LYSLSSVVTVPSSSLGTQT | | ARESRVSFEA | | PPSDEQLKSGTA | | SLQPEDFA |
| | | | YICNVNHKPSNTKVDKKV | | AMDYWGQG | | SVVCLLNNFYPR | | TYYCQQS |
| | | | EPKSCDKTHTCPPCPAPEL | | TLVTVSS | | EAKVQWKVDN | | EPEPYTFG |
| | | | LGGPSVFLFPPKPKDTLMI | | | | ALQSGNSQESVT | | QGTKVEI |
| | | | SRTPEVTCVVVDVSHEDP | | | | EQDSKDSTYSLS | | K |
| | | | EVKFNWYVDGVEVHNAK | | | | STLTLSKADYEK | | |
| | | | TKPREEQYNSTYRVVSVL | | | | HKVYACEVTHQ | | |
| | | | TVLHQDWLNGKEYKCKV | | | | GLSSPVTKSFNR | | |
| | | | SNKALPAPIEKTISKAKGQ | | | | GEC | | |
| | | | PREPQVYTLPPSRDELTKN | | | | | | |
| | | | QVSLWCLVKGFYPSDIAV | | | | | | |
| | | | EWESNGQPENNYKTTPPV | | | | | | |
| | | | LDSDGSFFLYSKLTVDKSR | | | | | | |
| | | | WQQGNVFSCSVMHEALH | | | | | | |
| | | | NHYTQKSLSLSPGK | | | | | | |
| EPI1045 | ITGB6 | 310 | QFQLVQSGAEVKKPGASV | 311 | QFQLVQSGAE | 312 | DIQMTQSPSSL | 313 | DIQMTQS |
| | | | KVSCKASGYSFTDYNVN | | VKKPGASVK | | SASVGDRVTITC | | PSSLSASV |
| | | | WVRQAPGQGLEWIGVINP | | VSCKASGYSF | | GASENIYGALN | | GDRVTITC |
| | | | KYGTTRYNQKFKGRATLT | | TDYNVNWVR | | WYQQKPGKAPK | | GASENIY |
| | | | VDKSTSTAYMELSSLRSED | | QAPGQGLEWI | | LLIYGATNLEDG | | GALNWY |
| | | | TAVYYCTRGLNAWDYWG | | GVINPKYGTT | | VPSRFSGSGSGR | | QQKPGKA |
| | | | QGTLVTVSSASTKGPSVFP | | RYNQKFKGR | | DYTFTISSLQPE | | PKLLIYGA |
| | | | LAPSSKSTSGGTAALGCLV | | ATLTVDKSTS | | DIATYYCQNVL | | TNLEDGV |
| | | | KDYFPEPVTVSWNSGALT | | TAYMELSSLR | | TTPYTFGQGTKL | | PSRFSGSG |
| | | | SGVHTFPAVLQSSGLYSLS | | SEDTAVYYCT | | EIKRTVAAPSVFI | | SGRDYTF |
| | | | SVVTVPSSSLGTQTYICNV | | RGLNAWDY | | FPPSDEQLKSGT | | TISSLQPE |
| | | | NHKPSNTKVDKKVEPKSC | | WGQGTLVTV | | ASVVCLLNNFY | | DIATYYC |
| | | | DKTHTCPPCPAPELLGGPS | | SS | | PREAKVQWKVD | | QNVLTTP |
| | | | VFLFPPKPKDTLMISRTPE | | | | NALQSGNSQES | | YTFGQGT |
| | | | VTCVVVDVSHEDPEVKFN | | | | VTEQDSKDSTYS | | KLEIK |
| | | | WYVDGVEVHNAKTKPRE | | | | LSSTLTLSKADY | | |
| | | | EQYNSTYRVVSVLTVLHQ | | | | EKHKVYACEVT | | |
| | | | DWLNGKEYKCKVSNKAL | | | | HQGLSSPVTKSF | | |
| | | | PAPIEKTISKAKGQPREPQ | | | | NRGEC | | |
| | | | VYTLPPSRDELTKNQVSL | | | | | | |
| | | | WCLVKGFYPSDIAVEWES | | | | | | |
| | | | NGQPENNYKTTPPVLDSD | | | | | | |
| | | | GSFFLYSKLTVDKSRWQQ | | | | | | |
| | | | GNVFSCSVMHEALHNHYT | | | | | | |
| | | | QKSLSLSPGK | | | | | | |
| EPI1094 | CD71 | 314 | EVQLVQSGAEVKKPGASV | 315 | EVQLVQSGAE | 316 | DIQMTQSPSSL | 317 | DIQMTQS |
| | | | KVSCKASGYTFTSYWMH | | VKKPGASVK | | SASVGDRVTITC | | PSSLSASV |
| | | | WVRQAPGQRLEWIGEINP | | VSCKASGYTF | | RASDNLYSNLA | | GDRVTITC |
| | | | TNGRTNYIEKFKSRATLTV | | TSYWMHWV | | WYQQKPGKSPK | | RASDNLY |
| | | | DKSASTAYMELSSLRSEDT | | RQAPGQRLE | | LLVYDATNLAD | | SNLAWYQ |
| | | | AVYYCARGTRAYHYWGQ | | WIGEINPTNG | | GVPSRFSGSGSG | | QKPGKSP |
| | | | GTMVTVSSASTKGPSVFPL | | RTNYIEKFKS | | TDYTLTISSLQPE | | KLLVYDA |
| | | | APSSKSTSGGTAALGCLV | | RATLTVDKSA | | DFATYYCQHFA | | TNLADGV |
| | | | KDYFPEPVTVSWNSGALT | | STAYMELSSL | | GTPLTFGQGTK | | PSRFSGSG |
| | | | SGVHTFPAVLQSSGLYSLS | | RSEDTAVYYC | | VEIKRTVAAPSV | | SGTDYTL |
| | | | SVVTVPSSSLGTQTYICNV | | ARGTRAYHY | | FIFPPSDEQLKSG | | TISSLQPE |
| | | | NHKPSNTKVDKKVEPKSC | | WGQGTMVTV | | TASVVCLLNNF | | DFATYYC |
| | | | DKTHTCPPCPAPELLGGPS | | SS | | YPREAKVQWKV | | QHFAGTP |
| | | | VFLFPPKPKDTLMISRTPE | | | | DNALQSGNSQE | | LTFGQGT |
| | | | VTCVVVDVSHEDPEVKFN | | | | SVTEQDSKDSTY | | KVEIK |
| | | | WYVDGVEVHNAKTKPRE | | | | SLSSTLTLSKAD | | |
| | | | EQYNSTYRVVSVLTVLHQ | | | | YEKHKVYACEV | | |
| | | | DWLNGKEYKCKVSNKAL | | | | THQGLSSPVTKS | | |
| | | | PAPIEKTISKAKGQPREPQ | | | | FNRGEC | | |
| | | | VYTLPPSRDELTKNQVSL | | | | | | |
| | | | WCLVKGFYPSDIAVEWES | | | | | | |
| | | | NGQPENNYKTTPPVLDSD | | | | | | |
| | | | GSFFLYSKLTVDKSRWQQ | | | | | | |
| | | | GNVFSCSVMHEALHNHYT | | | | | | |
| | | | QKSLSLSPGK | | | | | | |
| EPI1095 | CD71 | 318 | EVQLVQSGAEVKKPGASV | 319 | EVQLVQSGAE | 320 | DIQMTQSPSSL | 321 | DIQMTQS |
| | | | KVSCKASGYTFTSYWMH | | VKKPGASVK | | SASVGDRVTITC | | PSSLSASV |
| | | | WVRQAPGQRLEWIGEIAP | | VSCKASGYTF | | RASDNLYSNLA | | GDRVTITC |
| | | | TNGRTNYIEKFKSRATLTV | | TSYWMHWV | | WYQQKPGKSPK | | RASDNLY |
| | | | DKSASTAYMELSSLRSEDT | | RQAPGQRLE | | LLVYDATNLAD | | SNLAWYQ |
| | | | AVYYCARGTRAYHYWGQ | | WIGEIAPTNG | | GVPSRFSGSGSG | | QKPGKSP |
| | | | GTMVTVSSASTKGPSVFPL | | RTNYIEKFKS | | TDYTLTISSLQPE | | KLLVYDA |
| | | | APSSKSTSGGTAALGCLV | | RATLTVDKSA | | DFATYYCQHFA | | TNLADGV |
| | | | KDYFPEPVTVSWNSGALT | | STAYMELSSL | | GTPLTFGQGTK | | PSRFSGSG |
| | | | SGVHTFPAVLQSSGLYSLS | | RSEDTAVYYC | | VEIKRTVAAPSV | | SGTDYTL |
| | | | SVVTVPSSSLGTQTYICNV | | ARGTRAYHY | | FIFPPSDEQLKSG | | TISSLQPE |
| | | | NHKPSNTKVDKKVEPKSC | | WGQGTMVTV | | TASVVCLLNNF | | DFATYYC |
| | | | DKTHTCPPCPAPELLGGPS | | SS | | YPREAKVQWKV | | QHFAGTP |
| | | | VFLFPPKPKDTLMISRTPE | | | | DNALQSGNSQE | | LTFGQGT |
| | | | VTCVVVDVSHEDPEVKFN | | | | SVTEQDSKDSTY | | KVEIK |
| | | | WYVDGVEVHNAKTKPRE | | | | SLSSTLTLSKAD | | |
| | | | EQYNSTYRVVSVLTVLHQ | | | | YEKHKVYACEV | | |
| | | | DWLNGKEYKCKVSNKAL | | | | THQGLSSPVTKS | | |
| | | | PAPIEKTISKAKGQPREPQ | | | | FNRGEC | | |
| | | | VYTLPPSRDELTKNQVSL | | | | | | |
| | | | WCLVKGFYPSDIAVEWES | | | | | | |
| | | | NGQPENNYKTTPPVLDSD | | | | | | |
| | | | GSFFLYSKLTVDKSRWQQ | | | | | | |
| | | | GNVFSCSVMHEALHNHYT | | | | | | |
| | | | QKSLSLSPGK | | | | | | |
| EPI1485 | MSLN | 322 | EVQLQQSGPVLVKPGASV | 323 | EVQLQQSGPV | 324 | QAVVTQESAL | 325 | QAVVTQE |
| | | | KISCKASGYSFTGYYMHW | | LVKPGASVKI | | TTSPGETVTLTC | | SALTTSPG |
| | | | VRQSLVKRLEWIGRINPYT | | SCKASGYSFT | | RSSTGAVTTGN | | ETVTLTC |
| | | | GVPSYKHNFKDKASLTVD | | GYYMHWVR | | YPNWVQEKPDH | | RSSTGAV |
| | | | KSSSTAYMELHSLTSEDSA | | QSLVKRLEWI | | LFTGLIAGTNNR | | TTGNYPN |
| | | | VYYCARELGGYWGQGTT | | GRINPYTGVP | | APGVPARFSGSL | | WVQEKPD |
| | | | LTVSSASTKGPSVFPLAPSS | | SYKHNFKDK | | IGDKAALTITGA | | HLFTGLIA |
| | | | KSTSGGTAALGCLVKDYF | | ASLTVDKSSS | | QTEDEAIYFCAL | | GTNNRAP |
| | | | PEPVTVSWNSGALTSGVH | | TAYMELHSLT | | WFSSHWVFGGG | | GVPARFS |
| | | | TFPAVLQSSGLYSLSSVVT | | SEDSAVYYCA | | TKLTVLRTVAA | | GSLIGDK |
| | | | VPSSSLGTQTYICNVNHKP | | RELGGYWGQ | | PSVFIFPPSDEQL | | AALTITGA |
| | | | SNTKVDKKVEPKSCDKTH | | GTTLTVSS | | KSGTASVVCLL | | QTEDEAIY |
| | | | TCPPCPAPELLGGPSVFLFP | | | | NNFYPREAKVQ | | FCALWFS |
| | | | PKPKDTLMISRTPEVTCVV | | | | WKVDNALQSG | | SHWVFGG |
| | | | VDVSHEDPEVKFNWYVD | | | | NSQESVTEQDSK | | GTKLTVL |
| | | | GVEVHNAKTKPREEQYNS | | | | DSTYSLSSTLTL | | |
| | | | TYRVVSVLTVLHQDWLN | | | | SKADYEKHKVY | | |
| | | | GKEYKCKVSNKALPAPIE | | | | ACEVTHQGLSSP | | |
| | | | KTISKAKGQPREPQVYTLP | | | | VTKSFNRGEC | | |
| | | | PSRDELTKNQVSLWCLVK | | | | | | |
| | | | GFYPSDIAVEWESNGQPE | | | | | | |
| | | | NNYKTTPPVLDSDGSFFLY | | | | | | |
| | | | SKLTVDKSRWQQGNVFSC | | | | | | |
| | | | SVMHEALHNHYTQKSLSL | | | | | | |
| | | | SPGK | | | | | | |
| EPI1486 | MSLN | 326 | EVQLVESGGGLVQPGGSL | 327 | EVQLVESGGG | 328 | DIQMTQSPSSL | 329 | DIQMTQS |
| | | | RLSCAASGFTFSDYFMSW | | LVQPGGSLRL | | SASVGDRVTITC | | PSSLSASV |
| | | | VRQAPGKGLEWVATISNG | | SCAASGFTFS | | RASQDISNYLN | | GDRVTITC |
| | | | GTYTYYPDSVKGRFTISRD | | DYFMSWVRQ | | WYQQKPGKAPK | | RASQDISN |
| | | | NSKNTLYLQMNSLRAEDT | | APGKGLEWV | | LLIYYTSRLHSG | | YLNWYQ |
| | | | AVYYCARFDGYIFDYWG | | ATISNGGTYT | | VPSRFSGSGSGT | | QKPGKAP |
| | | | QGTLVTVSSASTKGPSVFP | | YYPDSVKGRF | | DFTLTISSLQPED | | KLLIYYTS |
| | | | LAPSSKSTSGGTAALGCLV | | TISRDNSKNT | | FATYYCQQGNT | | RLHSGVP |
| | | | KDYFPEPVTVSWNSGALT | | LYLQMNSLR | | LPYTFGQGTKV | | SRFSGSGS |
| | | | SGVHTFPAVLQSSGLYSLS | | AEDTAVYYC | | EIKRTVAAPSVFI | | GTDFTLTI |
| | | | SVVTVPSSSLGTQTYICNV | | ARFDGYIFDY | | FPPSDEQLKSGT | | SSLQPEDF |
| | | | NHKPSNTKVDKKVEPKSC | | WGQGTLVTV | | ASVVCLLNNFY | | ATYYCQQ |
| | | | DKTHTCPPCPAPELLGGPS | | SS | | PREAKVQWKVD | | GNTLPYT |
| | | | VFLFPPKPKDTLMISRTPE | | | | NALQSGNSQES | | FGQGTKV |
| | | | VTCVVVDVSHEDPEVKFN | | | | VTEQDSKDSTYS | | EIK |
| | | | WYVDGVEVHNAKTKPRE | | | | LSSTLTLSKADY | | |
| | | | EQYNSTYRVVSVLTVLHQ | | | | EKHKVYACEVT | | |
| | | | DWLNGKEYKCKVSNKAL | | | | HQGLSSPVTKSF | | |
| | | | PAPIEKTISKAKGQPREPQ | | | | NRGEC | | |
| | | | VYTLPPSRDELTKNQVSL | | | | | | |
| | | | WCLVKGFYPSDIAVEWES | | | | | | |
| | | | NGQPENNYKTTPPVLDSD | | | | | | |
| | | | GSFFLYSKLTVDKSRWQQ | | | | | | |
| | | | GNVFSCSVMHEALHNHYT | | | | | | |
| | | | QKSLSLSPGK | | | | | | |
| EPI1487 | RNF43 | 330 | QVQLVQSGAEVKKPGAS | 331 | QVQLVQSGA | 332 | DIQMTQSPSSL | 333 | DIQMTQS |
| | | | VKVSCKASGYTFTRYWIE | | EVKKPGASV | | SASVGDRVTITC | | PSSLSASV |
| | | | WVRQAPGQRLEWMGEILP | | KVSCKASGYT | | KASEDIYNRLA | | GDRVTITC |
| | | | GSGSTNYNEKFKGRVTITA | | FTRYWIEWV | | WYQQKPGKAPK | | KASEDIY |
| | | | DTSASTAYMELSSLRSEDT | | RQAPGQRLE | | LLISGATSLETG | | NRLAWY |
| | | | AVYYCERRGAYWGQGTL | | WMGEILPGSG | | VPSRFSGSGSGT | | QQKPGKA |
| | | | VTVSSASTKGPSVFPLAPS | | STNYNEKFKG | | DYTLTISSLQPE | | PKLLISGA |
| | | | SKSTSGGTAALGCLVKDY | | RVTITADTSA | | DFATYYCQQQW | | TSLETGVP |
| | | | FPEPVTVSWNSGALTSGV | | STAYMELSSL | | STPPTFGGGTKV | | SRFSGSGS |
| | | | HTFPAVLQSSGLYSLSSVV | | RSEDTAVYYC | | EIKRTVAAPSVFI | | GTDYTLTI |
| | | | TVPSSSLGTQTYICNVNHK | | ERRGAYWGQ | | FPPSDEQLKSGT | | SSLQPEDF |
| | | | PSNTKVDKKVEPKSCDKT | | GTLVTVSS | | ASVVCLLNNFY | | ATYYCQQ |
| | | | HTCPPCPAPELLGGPSVFL | | | | PREAKVQWKVD | | QWSTPPT |
| | | | FPPKPKDTLMISRTPEVTC | | | | NALQSGNSQES | | FGGGTKV |
| | | | VVVDVSHEDPEVKFNWY | | | | VTEQDSKDSTYS | | EIK |
| | | | VDGVEVHNAKTKPREEQY | | | | LSSTLTLSKADY | | |
| | | | NSTYRVVSVLTVLHQDWL | | | | EKHKVYACEVT | | |
| | | | NGKEYKCKVSNKALPAPI | | | | HQGLSSPVTKSF | | |
| | | | EKTISKAKGQPREPQVYTL | | | | NRGEC | | |
| | | | PPSRDELTKNQVSLWCLV | | | | | | |
| | | | KGFYPSDIAVEWESNGQP | | | | | | |
| | | | ENNYKTTPPVLDSDGSFFL | | | | | | |
| | | | YSKLTVDKSRWQQGNVFS | | | | | | |
| | | | CSVMHEALHNHYTQKSLS | | | | | | |
| | | | LSPGK | | | | | | |
| EPI1550 | ITGB6 | 334 | QVQLVQSGAEVKKPGAS | 335 | QVQLVQSGA | 336 | DVVMTQSPLS | 337 | DVVMTQS |
| | | | VKVSCKASGYSFSGYFMN | | EVKKPGASV | | LPVTLGQPASIS | | PLSLPVTL |
| | | | WVRQAPGQGLEWMGLIN | | KVSCKASGYS | | CKSSQSLLDSDG | | GQPASISC |
| | | | PYNGDSFYNQKFKGRVTM | | FSGYFMNWV | | KTYLNWLFQRP | | KSSQSLLD |
| | | | TRQTSTSTVYMELSSLRSE | | RQAPGQGLE | | GQSPRRLIYLVS | | SDGKTYL |
| | | | DTAVYYCVRGLRRDFDY | | WMGLINPYN | | ELDSGVPDRFSG | | NWLFQRP |
| | | | WGQGTLVTVSSASTKGPS | | GDSFYNQKFK | | SGSGTDFTLKIS | | GQSPRRLI |
| | | | VFPLAPSSKSTSGGTAALG | | GRVTMTRQT | | RVEAEDVGVYY | | YLVSELD |
| | | | CLVKDYFPEPVTVSWNSG | | STSTVYMELS | | CWQGTHFPRTF | | SGVPDRFS |
| | | | ALTSGVHTFPAVLQSSGLY | | SLRSEDTAVY | | GGGTKLEIKRTV | | GSGSGTD |
| | | | SLSSVVTVPSSSLGTQTYIC | | YCVRGLRRDF | | AAPSVFIFPPSDE | | FTLKISRV |
| | | | NVNHKPSNTKVDKKVEPK | | DYWGQGTLV | | QLKSGTASVVC | | EAEDVGV |
| | | | SCDKTHTCPPCPAPELLGG | | TVSS | | LLNNFYPREAK | | YYCWQG |
| | | | PSVFLFPPKPKDTLMISRTP | | | | VQWKVDNALQ | | THFPRTFG |
| | | | EVTCVVVDVSHEDPEVKF | | | | SGNSQESVTEQD | | GGTKLEIK |
| | | | NWYVDGVEVHNAKTKPR | | | | SKDSTYSLSSTL | | |
| | | | EEQYNSTYRVVSVLTVLH | | | | TLSKADYEKHK | | |
| | | | QDWLNGKEYKCKVSNKA | | | | VYACEVTHQGL | | |
| | | | LPAPIEKTISKAKGQPREPQ | | | | SSPVTKSFNRGE | | |
| | | | VYTLPPSRDELTKNQVSL | | | | C | | |
| | | | WCLVKGFYPSDIAVEWES | | | | | | |
| | | | NGQPENNYKTTPPVLDSD | | | | | | |
| | | | GSFFLYSKLTVDKSRWQQ | | | | | | |
| | | | GNVFSCSVMHEALHNHYT | | | | | | |
| | | | QKSLSLSPGK | | | | | | |
| EPI1617 | EGFR | 338 | EVQLVESGGGLVQPGGSL | 339 | EVQLVESGGG | 340 | DIQMTQSPSSL | 341 | DIQMTQS |
| | | | RLSCAASGFTFTGNWIHW | | LVQPGGSLRL | | SASVGDRVTITC | | PSSLSASV |
| | | | VRQAPGKGLEWVGEISPS | | SCAASGFTFT | | RASQDVSTAVA | | GDRVTITC |
| | | | GGYTDYADSVKGRFTISA | | GNWIHWVRQ | | WYQQKPGKAPK | | RASQDVS |
| | | | DTSKNTAYLQMNSLRAED | | APGKGLEWV | | LLIYSASFLYSG | | TAVAWY |
| | | | TAVYYCARESRVSYEAAM | | GEISPSGGYT | | VPSRFSGSGSGT | | QQKPGKA |
| | | | DYWGQGTLVTVSSASTKG | | DYADSVKGR | | DFTLTISSLQPED | | PKLLIYSA |
| | | | PSVFPLAPSSKSTSGGTAA | | FTISADTSKNT | | FATYYCQQSYP | | SFLYSGVP |
| | | | LGCLVKDYFPEPVTVSWN | | AYLQMNSLR | | TPYTFGQGTKV | | SRFSGSGS |
| | | | SGALTSGVHTFPAVLQSSG | | AEDTAVYYC | | EIKRTVAAPSVFI | | GTDFTLTI |
| | | | LYSLSSVVTVPSSSLGTQT | | ARESRVSYEA | | FPPSDEQLKSGT | | SSLQPEDF |
| | | | YICNVNHKPSNTKVDKKV | | AMDYWGQG | | ASVVCLLNNFY | | ATYYCQQ |
| | | | EPKSCDKTHTCPPCPAPEL | | TLVTVSS | | PREAKVQWKVD | | SYPTPYTF |
| | | | LGGPSVFLFPPKPKDTLMI | | | | NALQSGNSQES | | GQGTKVE |
| | | | SRTPEVTCVVVDVSHEDP | | | | VTEQDSKDSTYS | | IK |
| | | | EVKFNWYVDGVEVHNAK | | | | LSSTLTLSKADY | | |
| | | | TKPREEQYNSTYRVVSVL | | | | EKHKVYACEVT | | |
| | | | TVLHQDWLNGKEYKCKV | | | | HQGLSSPVTKSF | | |
| | | | SNKALPAPIEKTISKAKGQ | | | | NRGEC | | |
| | | | PREPQVYTLPPSRDELTKN | | | | | | |
| | | | QVSLWCLVKGFYPSDIAV | | | | | | |
| | | | EWESNGQPENNYKTTPPV | | | | | | |
| | | | LDSDGSFFLYSKLTVDKSR | | | | | | |
| | | | WQQGNVFSCSVMHEALH | | | | | | |
| | | | NHYTQKSLSLSPGK | | | | | | |
| EPI1625 | ITGB6 | 342 | QVQLQQSGAELVRPGTSV | 343 | QVQLQQSGA | 344 | DIVMTQSHKF | 345 | DIVMTQS |
| | | | KVSCKASGYDFNNDLIEW | | ELVRPGTSVK | | MSTVVGDRVSIT | | HKFMSTV |
| | | | VKQRPGQGLEWIAVINPG | | VSCKASGYDF | | CKASLDVRTAV | | VGDRVSIT |
| | | | SGRTNYNEKFKGKATLTA | | NNDLIEWVK | | AWYQQKPGQSP | | CKASLDV |
| | | | DKSSSTVYMQLSSLTSDDS | | QRPGQGLEWI | | KLLIYSASYRYT | | RTAVAW |
| | | | AVYFCAMIYYGPHSYAM | | AVINPGSGRT | | GVPDRFTGSGSG | | YQQKPGQ |
| | | | DYWGQGTSVTVSSASTKG | | NYNEKFKGK | | TDFTFNIRSVQA | | SPKLLIYS |
| | | | PSVFPLAPSSKSTSGGTAA | | ATLTADKSSS | | EDLAVYYCQQH | | ASYRYTG |
| | | | LGCLVKDYFPEPVTVSWN | | TVYMQLSSLT | | YGIPWTFGGGT | | VPDRFTG |
| | | | SGALTSGVHTFPAVLQSSG | | SDDSAVYFCA | | KLEIKRTVAAPS | | SGSGTDFT |
| | | | LYSLSSVVTVPSSSLGTQT | | MIYYGPHSYA | | VFIFPPSDEQLKS | | FNIRSVQA |
| | | | YICNVNHKPSNTKVDKKV | | MDYWGQGTS | | GTASVVCLLNN | | EDLAVYY |
| | | | EPKSCDKTHTCPPCPAPEL | | VTVSS | | FYPREAKVQWK | | CQQHYGI |
| | | | LGGPSVFLFPPKPKDTLMI | | | | VDNALQSGNSQ | | PWTFGGG |
| | | | SRTPEVTCVVVDVSHEDP | | | | ESVTEQDSKDST | | TKLEIK |
| | | | EVKFNWYVDGVEVHNAK | | | | YSLSSTLTLSKA | | |
| | | | TKPREEQYNSTYRVVSVL | | | | DYEKHKVYACE | | |
| | | | TVLHQDWLNGKEYKCKV | | | | VTHQGLSSPVTK | | |
| | | | SNKALPAPIEKTISKAKGQ | | | | SFNRGEC | | |
| | | | PREPQVYTLPPSRDELTKN | | | | | | |
| | | | QVSLWCLVKGFYPSDIAV | | | | | | |
| | | | EWESNGQPENNYKTTPPV | | | | | | |
| | | | LDSDGSFFLYSKLTVDKSR | | | | | | |
| | | | WQQGNVFSCSVMHEALH | | | | | | |
| | | | NHYTQKSLSLSPGK | | | | | | |
| EPI1626 | ITGB6 | 346 | QVQLQQSGAELARPGTSV | 347 | QVQLQQSGA | 348 | DIVMTQSHKF | 349 | DIVMTQS |
| | | | KVSCKASGYAFTNYLIEW | | ELARPGTSVK | | MSTSVGDRVSV | | HKFMSTS |
| | | | VKQRPGQGLEWIGVISPGS | | VSCKASGYAF | | TCKASQAVNTA | | VGDRVSV |
| | | | GIINYNEKFKGKATLTADK | | TNYLIEWVKQ | | VAWYQQKPGQS | | TCKASQA |
| | | | SSSTAYMQLSSLTSDDSAV | | RPGQGLEWIG | | PKLLIYSASYGY | | VNTAVA |
| | | | YFCAAIDYSGPYAVDDWG | | VISPGSGIINY | | TGVPDRFTGSGS | | WYQQKP |
| | | | QGTSVTVSSASTKGPSVFP | | NEKFKGKATI | | GTDFTLTISSVQ | | GQSPKLLI |
| | | | LAPSSKSTSGGTAALGCLV | | TADKSSSTAY | | AEDLAVYYCQH | | YSASYGY |
| | | | KDYFPEPVTVSWNSGALT | | MQLSSLTSDD | | HYGVPWTFGGG | | TGVPDRF |
| | | | SGVHTFPAVLQSSGLYSLS | | SAVYFCAAID | | TKLEIKRTVAAP | | TGSGSGT |
| | | | SVVTVPSSSLGTQTYICNV | | YSGPYAVDD | | SVFIFPPSDEQLK | | DFTLTISS |
| | | | NHKPSNTKVDKKVEPKSC | | WGQGTSVTV | | SGTASVVCLLN | | VQAEDLA |
| | | | DKTHTCPPCPAPELLGGPS | | SS | | NFYPREAKVQW | | VYYCQHH |
| | | | VFLFPPKPKDTLMISRTPE | | | | KVDNALQSGNS | | YGVPWTF |
| | | | VTCVVVDVSHEDPEVKFN | | | | QESVTEQDSKDS | | GGGTKLEI |
| | | | WYVDGVEVHNAKTKPRE | | | | TYSLSSTLTLSK | | K |
| | | | EQYNSTYRVVSVLTVLHQ | | | | ADYEKHKVYAC | | |
| | | | DWLNGKEYKCKVSNKAL | | | | EVTHQGLSSPVT | | |
| | | | PAPIEKTISKAKGQPREPQ | | | | KSFNRGEC | | |
| | | | VYTLPPSRDELTKNQVSL | | | | | | |
| | | | WCLVKGFYPSDIAVEWES | | | | | | |
| | | | NGQPENNYKTTPPVLDSD | | | | | | |
| | | | GSFFLYSKLTVDKSRWQQ | | | | | | |
| | | | GNVFSCSVMHEALHNHYT | | | | | | |
| | | | QKSLSLSPGK | | | | | | |
| EPI1720 | RSV F | 350 | QVTLRESGPALVKPTQTL | 351 | QVTLRESGPA | 352 | DIQMTQSPSTL | 353 | DIQMTQS |
| | Pro- | | TLTCTFSGFSLSTSGMSVG | | LVKPTQTLTL | | SASVGDRVTITC | | PSTLSASV |
| | tein | | WIRQPPGKALEWLADIW | | TCTFSGFSLST | | KCQLSVGYMH | | GDRVTITC |
| | | | WDDKKDYNPSLKSRLTIS | | SGMSVGWIR | | WYQQKPGKAPK | | KCQLSVG |
| | | | KDTSKNQVVLKVTNMDP | | QPPGKALEW | | LLIYDTSKLASG | | YMHWYQ |
| | | | ADTATYYCARSMITNWYF | | LADIWWDDK | | VPSRFSGSGSGT | | QKPGKAP |
| | | | DVWGAGTTVTVSSASTKG | | KDYNPSLKSR | | EFTLTISSLQPDD | | KLLIYDTS |
| | | | PSVFPLAPSSKSTSGGTAA | | LTISKDTSKN | | FATYYCFQGSG | | KLASGVP |
| | | | LGCLVKDYFPEPVTVSWN | | QVVLKVTNM | | YPFTFGGGTKLE | | SRFSGSGS |
| | | | SGALTSGVHTFPAVLQSSG | | DPADTATYY | | IKRTVAAPSVFIF | | GTEFTLTI |
| | | | LYSLSSVVTVPSSSLGTQT | | CARSMITNW | | PPSDEQLKSGTA | | SSLQPDDF |
| | | | YICNVNHKPSNTKVDKKV | | YFDVWGAGT | | SVVCLLNNFYPR | | ATYYCFQ |
| | | | EPKSCDKTHTCPPCPAPEL | | TVTVSS | | EAKVQWKVDN | | GSGYPFTF |
| | | | LGGPSVFLFPPKPKDTLMI | | | | ALQSGNSQESVT | | GGGTKLEI |
| | | | SRTPEVTCVVVDVSHEDP | | | | EQDSKDSTYSLS | | K |
| | | | EVKFNWYVDGVEVHNAK | | | | STLTLSKADYEK | | |
| | | | TKPREEQYNSTYRVVSVL | | | | HKVYACEVTHQ | | |
| | | | TVLHQDWLNGKEYKCKV | | | | GLSSPVTKSFNR | | |
| | | | SNKALPAPIEKTISKAKGQ | | | | GEC | | |
| | | | PREPQVYTLPPSRDELTKN | | | | | | |
| | | | QVSLWCLVKGFYPSDIAV | | | | | | |
| | | | EWESNGQPENNYKTTPPV | | | | | | |
| | | | LDSDGSFFLYSKLTVDKSR | | | | | | |
| | | | WQQGNVFSCSVMHEALH | | | | | | |
| | | | NHYTQKSLSLSPGK | | | | | | |

The sequences listed in Table 1 (SEQ ID NOs: 1-353) are amino acid molecules. The sequences listed in Table 1 (SEQ ID NOs: 1-353) are amino acid molecules that are synthetic constructs. The sequences listed in Table 1 (SEQ ID NOs: 1-353) for HC sequences (heavy chain), VH sequence (variable heavy chain sequence), LC sequences (light chain), VL sequence (variable light chain sequence) are amino acid molecules that are synthetic constructs.

In some embodiments, the antibodies targeting the internalizing receptor protein comprise a sequence listed Table 2. In some embodiments, the antibodies targeting the internalizing receptor protein comprise at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, at least 99.9%, or at least 99.9% sequence identity to a sequence listed Table 2.

In some cases, the antibodies targeting the internalizing receptor protein may bind the same epitope as any one of the antibodies listed in Table 2. The antibodies targeting the internalizing receptor protein may bind to an epitope that comprises about 70% sequence identity to the epitope to which any one of the antibodies listed in Table 2 binds. The antibodies targeting the internalizing receptor protein may bind to an epitope that comprises about 75% sequence identity to the epitope to which any one of the antibodies listed in Table 2 binds. The antibodies targeting the internalizing receptor protein may bind to an epitope that comprises about 80% sequence identity to the epitope to which any one of the antibodies listed in Table 2 binds. The antibodies targeting the internalizing receptor protein may bind to an epitope that comprises about 85% sequence identity to the epitope to which any one of the antibodies listed in Table 2 binds. The antibodies targeting the internalizing receptor protein may bind to an epitope that comprises about 90% sequence identity to the epitope to which any one of the antibodies listed in Table 2 binds. The antibodies targeting the internalizing receptor protein may bind to an epitope that comprises about 95% sequence identity to the epitope to which any one of the antibodies listed in Table 2 binds. The antibodies targeting the internalizing receptor protein may bind to an epitope that comprises about 99% sequence identity to the epitope to which any one of the antibodies listed in Table 2 binds.

In some embodiments, the antibodies targeting the internalizing receptor protein may bind with a similar affinity as any one of the antibodies listed in Table 2 (Table 5 lists affinities of certain monovalent binders). Table 5 describes monovalent Kds to particular internalizing receptor monovalent proteins. In certain embodiments, multispecific binding agents have a Kd less than, more than, within 10%, within 20%, within 30%, within 40%, within 50%, withing 75%, or within 100% of the binding affinity of the monovalent binding agent. For example, in Table 5, the monovalent binding affinities are described for certain CD71 monovalent binding agents. When those CD71 binding arms are incorporated in the monovalent binding agent of the disclosure, the binding affinity of the multispecific binding agent may be within an order of magnitude or an order of two-fold as the binding affinity of the monovalent binding agent. For example, the binding affinity of the monovalent binding agent has a Kd of between 0.1 nM and 100 nM. When incorporated into the multispecific binding agent, the Kd may be within the same range. Alternatively, the binding affinity may be slightly greater than, but within two fold of the monovalent binding affinity. The binding affinity may be within three fold of the monovalent binding affinity.

In some embodiments, the antibodies targeting the internalizing receptor protein may bind the same epitope as any one of the antibodies listed in Table 2 binds with a similar affinity as any one of the antibodies listed in Table 2. The antibodies targeting the internalizing receptor protein may bind to an epitope that comprises about 70% sequence identity to the epitope to which any one of the antibodies listed in Table 2 binds with a similar affinity as any one of the antibodies listed in Table 2. The antibodies targeting the internalizing receptor protein may bind to an epitope that comprises about 75% sequence identity to the epitope to which any one of the antibodies listed in Table 2 binds with a similar affinity as any one of the antibodies listed in Table 2. The antibodies targeting the internalizing receptor protein may bind to an epitope that comprises about 80% sequence identity to the epitope to which any one of the antibodies listed in Table 2 binds with a similar affinity as any one of the antibodies listed in Table 2. The antibodies targeting the internalizing receptor protein may bind to an epitope that comprises about 85% sequence identity to the epitope to which any one of the antibodies listed in Table 2 binds with a similar affinity as any one of the antibodies listed in Table 2. The antibodies targeting the internalizing receptor protein may bind to an epitope that comprises about 90% sequence identity to the epitope to which any one of the antibodies listed in Table 2 binds with a similar affinity as any one of the antibodies listed in Table 2. The antibodies targeting the internalizing receptor protein may bind to an epitope that comprises about 95% sequence identity to the epitope to which any one of the antibodies listed in Table 2 binds with a similar affinity as any one of the antibodies listed in Table 2. The antibodies targeting the internalizing receptor protein may bind to an epitope that comprises about 99% sequence identity to the epitope to which any one of the antibodies listed in Table 2 binds with a similar affinity as any one of the antibodies listed in Table 2.

In some embodiments, the antibodies targeting the internalizing receptor protein may bind the same epitope as any one of the antibodies listed in Table 2 binds with a different affinity as compared to any one of the antibodies listed in Table 2. The antibodies targeting the internalizing receptor protein may bind to an epitope that comprises about 70% sequence identity to the epitope to which any one of the antibodies listed in Table 2 binds with a different affinity as compared to any one of the antibodies listed in Table 2. The antibodies targeting the internalizing receptor protein may bind to an epitope that comprises about 75% sequence identity to the epitope to which any one of the antibodies listed in Table 2 binds with a different affinity as compared to any one of the antibodies listed in Table 2. The antibodies targeting the internalizing receptor protein may bind to an epitope that comprises about 80% sequence identity to the epitope to which any one of the antibodies listed in Table 2 binds with a different affinity as compared to any one of the antibodies listed in Table 2. The antibodies targeting the internalizing receptor protein may bind to an epitope that comprises about 85% sequence identity to the epitope to which any one of the antibodies listed in Table 2 binds with a different affinity as compared to any one of the antibodies listed in Table 2. The antibodies targeting the internalizing receptor protein may bind to an epitope that comprises about 90% sequence identity to the epitope to which any one of the antibodies listed in Table 2 binds with a different affinity as compared to any one of the antibodies listed in Table 2. The antibodies targeting the internalizing receptor protein may bind to an epitope that comprises about 95% sequence identity to the epitope to which any one of the antibodies listed in Table 2 binds with a different affinity as compared to any one of the antibodies listed in Table 2. The antibodies targeting the internalizing receptor protein may bind to an epitope that comprises about 99% sequence identity to the epitope to which any one of the antibodies listed in Table 2 binds with a different affinity as compared to any one of the antibodies listed in Table 2.

The antibodies targeting the internalizing receptor protein may bind to an epitope that comprises a different epitope than the epitope to which any one of the antibodies listed in Table 2 binds. The antibodies targeting the internalizing receptor protein may bind to an epitope that comprises a different epitope than the epitope to which any one of the antibodies listed in Table 2 binds, wherein the epitopes do not bind to any of the same amino acids on the internalizing receptor protein. The antibodies targeting the internalizing receptor protein may bind to an epitope that comprises a different epitope than the epitope to which any one of the antibodies listed in Table 2 binds, wherein the epitopes bind to any one or more of the same amino acids on the internalizing receptor protein. The antibodies targeting the internalizing receptor protein may bind to an epitope that comprises a different epitope than the epitope to which any one of the antibodies listed in Table 2 binds, wherein the epitopes bind to any two or more of the same amino acids on the internalizing receptor protein. The antibodies targeting the internalizing receptor protein may bind to an epitope that comprises a different epitope than the epitope to which any one of the antibodies listed in Table 2 binds, wherein the epitopes bind to any three or more of the same amino acids on the internalizing receptor protein. The antibodies targeting the internalizing receptor protein may bind to an epitope that comprises a different epitope than the epitope to which any one of the antibodies listed in Table 2 binds, wherein the epitopes bind to any four or more of the same amino acids on the internalizing receptor protein. The antibodies targeting the internalizing receptor protein may bind to an epitope that comprises a different epitope than the epitope to which any one of the antibodies listed in Table 2 binds, wherein the epitopes bind to any five or more of the same amino acids on the internalizing receptor protein. The antibodies targeting the internalizing receptor protein may bind to an epitope that comprises a different epitope than the epitope to which any one of the antibodies listed in Table 2 binds, wherein the epitopes bind to any six or more of the same amino acids on the internalizing receptor protein. The antibodies targeting the internalizing receptor protein may bind to an epitope that comprises a different epitope than the epitope to which any one of the antibodies listed in Table 2 binds, wherein the epitopes bind to any seven or more of the same amino acids on the internalizing receptor protein. The antibodies targeting the internalizing receptor protein may bind to an epitope that comprises a different epitope than the epitope to which any one of the antibodies listed in Table 2 binds, wherein the epitopes bind to any eight or more of the same amino acids on the internalizing receptor protein. The antibodies targeting the internalizing receptor protein may bind to an epitope that comprises a different epitope than the epitope to which any one of the antibodies listed in Table 2 binds, wherein the epitopes bind to any nine or more of the same amino acids on the internalizing receptor protein. The antibodies targeting the internalizing receptor protein may bind to an epitope that comprises a different epitope than the epitope to which any one of the antibodies listed in Table 2 binds, wherein the epitopes bind to any ten or more of the same amino acids on the internalizing receptor protein.

In some embodiments, the antibodies targeting the degrader protein comprises a sequence listed Table 2. In some embodiments, the antibodies targeting the degrader protein comprise at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, at least 99.9%, or at least 99.9% sequence identity to a sequence listed Table 2.

In some cases, the antibodies targeting the degrader protein may bind the same epitope as any one of the antibodies listed in Table 2. The antibodies targeting the degrader protein may bind to an epitope that comprises about 70% sequence identity to the epitope to which any one of the antibodies listed in Table 2 binds. The antibodies targeting the degrader protein may bind to an epitope that comprises about 75% sequence identity to the epitope to which any one of the antibodies listed in Table 2 binds. The antibodies targeting the degrader protein may bind to an epitope that comprises about 80% sequence identity to the epitope to which any one of the antibodies listed in Table 2 binds. The antibodies targeting the degrader protein may bind to an epitope that comprises about 85% sequence identity to the epitope to which any one of the antibodies listed in Table 2 binds. The antibodies targeting the degrader protein may bind to an epitope that comprises about 90% sequence identity to the epitope to which any one of the antibodies listed in Table 2 binds. The antibodies targeting the degrader protein may bind to an epitope that comprises about 95% sequence identity to the epitope to which any one of the antibodies listed in Table 2 binds. The antibodies targeting the degrader protein may bind to an epitope that comprises about 99% sequence identity to the epitope to which any one of the antibodies listed in Table 2 binds.

The antibodies targeting the degrader protein may bind to an epitope that comprises a different epitope than the epitope to which any one of the antibodies listed in Table 2 binds. The antibodies targeting the degrader protein may bind to an epitope that comprises a different epitope than the epitope to which any one of the antibodies listed in Table 2 binds, wherein the epitopes do not bind to any of the same amino acids on the degrader protein. The antibodies targeting the degrader protein may bind to an epitope that comprises a different epitope than the epitope to which any one of the antibodies listed in Table 2 binds, wherein the epitopes bind to any one or more of the same amino acids on the degrader protein. The antibodies targeting the degrader protein may bind to an epitope that comprises a different epitope than the epitope to which any one of the antibodies listed in Table 2 binds, wherein the epitopes bind to any two or more of the same amino acids on the degrader protein. The antibodies targeting the degrader protein may bind to an epitope that comprises a different epitope than the epitope to which any one of the antibodies listed in Table 2 binds, wherein the epitopes bind to any three or more of the same amino acids on the degrader protein. The antibodies targeting the degrader protein may bind to an epitope that comprises a different epitope than the epitope to which any one of the antibodies listed in Table 2 binds, wherein the epitopes bind to any four or more of the same amino acids on the degrader protein. The antibodies targeting the degrader protein may bind to an epitope that comprises a different epitope than the epitope to which any one of the antibodies listed in Table 2 binds, wherein the epitopes bind to any five or more of the same amino acids on the degrader protein. The antibodies targeting the degrader protein may bind to an epitope that comprises a different epitope than the epitope to which any one of the antibodies listed in Table 2 binds, wherein the epitopes bind to any six or more of the same amino acids on the degrader protein. The antibodies targeting the degrader protein may bind to an epitope that comprises a different epitope than the epitope to which any one of the antibodies listed in Table 2 binds, wherein the epitopes bind to any seven or more of the same amino acids on the degrader protein. The antibodies targeting the degrader protein may bind to an epitope that comprises a different epitope than the epitope to which any one of the antibodies listed in Table 2 binds, wherein the epitopes bind to any eight or more of the same amino acids on the degrader protein. The antibodies targeting the degrader protein may bind to an epitope that comprises a different epitope than the epitope to which any one of the antibodies listed in Table 2 binds, wherein the epitopes bind to any nine or more of the same amino acids on the degrader protein. The antibodies targeting the degrader protein may bind to an epitope that comprises a different epitope than the epitope to which any one of the antibodies listed in Table 2 binds, wherein the epitopes bind to any ten or more of the same amino acids on the degrader protein.

TABLE 2
Additional exemplary antibody sequences targeting the internalizing receptor
protein or degrader protein.

| | | SEQ | | SEQ | | SEQ | | SEQ | |
|---|---|---|---|---|---|---|---|---|---|
| | Arm 1 | ID | | ID | VH | ID | | ID | VL |
| ID | Target | NO | HC sequence | NO | sequence | NO | LC sequence | NO | sequence |
| EPI107 | RNF43 | 354 | EVQLVESGGGLVQPGGSL | 355 | EVQLVESGG | 356 | DIQMTQSPSSLS | 357 | DIQMTQ |
| | | | RLSCAASGFNIYYYSMHW | | GLVQPGGSL | | ASVGDRVTITCR | | SPSSLSA |
| | | | VRQAPGKGLEWVASISPY | | RLSCAASGF | | ASQSVGSALAW | | SVGDRV |
| | | | YSYTSYADSVKGRFTISAD | | NIYYYSMH | | YQQKPGKAPKL | | TITCRAS |
| | | | TSKNTAYLQMNSLRAEDT | | WVRQAPGK | | LIYSASSLYSGV | | QSVGSA |
| | | | AVYYCARYGYYGWDYHR | | GLEWVASIS | | PSRFSGSRSGTD | | LAWYQ |
| | | | YSAFDYWGQGTLVTVSSA | | PYYSYTSYA | | FTLTISSLQPEDF | | QKPGKA |
| | | | STKGPSVFPLAPSSKSTSG | | DSVKGRFTI | | ATYYCQQAYPIT | | PKLLIYS |
| | | | GTAALGCLVKDYFPEPVT | | SADTSKNTA | | FGQGTKVEIKRT | | ASSLYS |
| | | | VSWNSGALTSGVHTFPAV | | YLQMNSLR | | VAAPSVFIFPPSD | | GVPSRFS |
| | | | LQSSGLYSLSSVVTVPSSS | | AEDTAVYY | | SQLKSGTASVV | | GSRSGT |
| | | | LGTQTYICNVNHKPSNTK | | CARYGYYG | | CLLNNFYPREA | | DFTLTIS |
| | | | VDKKVEPKSCDKTHTCPP | | WDYHRYSA | | KVQWKVDNAL | | SLQPEDF |
| | | | CPAPELLGGPSVFLFPPKP | | FDYWGQGT | | QSGNSQESVTEQ | | ATYYCQ |
| | | | KDTLMISRTPEVTCVVVD | | LVTVSS | | DSKDSTYSLSST | | QAYPITF |
| | | | VSHEDPEVKFNWYVDGV | | | | LTLSKADYEKH | | GQGTKV |
| | | | EVHNAKTKPREEQYNSTY | | | | KVYACEVTHQG | | EIK |
| | | | RVVSVLTVLHQDWLNGK | | | | LSSPVTKSFNRG | | |
| | | | EYKCKVSNKALPAPIEKTI | | | | EC + J3:J77 | | |
| | | | SKAKGQPREPQVYTLPPSR | | | | | | |
| | | | DELTKNQVSLWCLVKGFY | | | | | | |
| | | | PSDIAVEWESNGQPENNY | | | | | | |
| | | | KTTPPVLDSDGSFFLYSKL | | | | | | |
| | | | TVDKSRWQQGNVFSCSV | | | | | | |
| | | | MHEALHNHYTQKSLSLSP | | | | | | |
| | | | GKGGSHHHHHH | | | | | | |
| EPI112 | ZNRF3 | 358 | EVQLVESGGGLVQPGGSL | 359 | EVQLVESGG | 360 | DIQMTQSPSSLS | 361 | DIQMTQ |
| | | | RLSCAASGFNLYYSYIHW | | GLVQPGGSL | | ASVGDRVTITCR | | SPSSLSA |
| | | | VRQAPGKGLEWVASIYPS | | RLSCAASGF | | ASQSVGSALAW | | SVGDRV |
| | | | YGSTYYADSVKGRFTISRD | | NLYYSYIHW | | YQQKPGKAPKL | | TITCRAS |
| | | | NSKNTLYLQMNSLRAEDT | | VRQAPGKG | | LIYSASSLYSGV | | QSVGSA |
| | | | AVYYCARGYAIDYWGQG | | LEWVASIYP | | PSRFSGSRSGTD | | LAWYQ |
| | | | TLVTVSSASTKGPSVFPLA | | SYGSTYYAD | | FTLTISSLQPEDF | | QKPGKA |
| | | | PSSKSTSGGTAALGCLVK | | SVKGRFTIS | | ATYYCQQSYYPI | | PKLLIYS |
| | | | DYFPEPVTVSWNSGALTS | | RDNSKNTLY | | TFGQGTKVEIKR | | ASSLYS |
| | | | GVHTFPAVLQSSGLYSLSS | | LQMNSLRA | | TVAAPSVFIFPPS | | GVPSRFS |
| | | | VVTVPSSSLGTQTYICNVN | | EDTAVYYC | | DSQLKSGTASV | | GSRSGT |
| | | | HKPSNTKVDKKVEPKSCD | | ARGYAIDY | | VCLLNNFYPRE | | DFTLTIS |
| | | | KTHTCPPCPAPELLGGPSV | | WGQGTLVT | | AKVQWKVDNA | | SLQPEDF |
| | | | FLFPPKPKDTLMISRTPEVT | | VSS | | LQSGNSQESVTE | | ATYYCQ |
| | | | CVVVDVSHEDPEVKFNW | | | | QDSKDSTYSLSS | | QSYYPIT |
| | | | YVDGVEVHNAKTKPREEQ | | | | TLTLSKADYEK | | FGQGTK |
| | | | YNSTYRVVSVLTVLHQD | | | | HKVYACEVTHQ | | VEIK |
| | | | WLNGKEYKCKVSNKALP | | | | GLSSPVTKSFNR | | |
| | | | APIEKTISKAKGQPREPQV | | | | GEC | | |
| | | | YTLPPSRDELTKNQVSLW | | | | | | |
| | | | CLVKGFYPSDIAVEWESN | | | | | | |
| | | | GQPENNYKTTPPVLDSDG | | | | | | |
| | | | SFFLYSKLTVDKSRWQQG | | | | | | |
| | | | NVFSCSVMHEALHNHYTQ | | | | | | |
| | | | KSLSLSPGKGGSHHHHHH | | | | | | |
| EPI132 | | 362 | EVQLVESGGGLVQPGGSL | 363 | EVQLVESGG | | | | |
| | | | RLSCAASGFRIYSSSYYIG | | GLVQPGGSL | | | | |
| | | | WVRRAPGKGEELVARIYP | | RLSCAASGF | | | | |
| | | | SSGSTYYADSVKGRFTISA | | RIYSSSYYIG | | | | |
| | | | DTSKNTAYLQMNSLRAED | | WVRRAPGK | | | | |
| | | | TAVYYCARYAVGYGYPW | | GEELVARIY | | | | |
| | | | YGWGLDYWGQGTLVTVS | | PSSGSTYYA | | | | |
| | | | SEPKSCDKTHTCPPCPAPE | | DSVKGRFTI | | | | |
| | | | LLGGPSVFLFPPKPKDTLM | | SADTSKNTA | | | | |
| | | | ISRTPEVTCVVVDVSHEDP | | YLQMNSLR | | | | |
| | | | EVKFNWYVDGVEVHNAK | | AEDTAVYY | | | | |
| | | | TKPREEQYNSTYRVVSVL | | CARYAVGY | | | | |
| | | | TVLHQDWLNGKEYKCKV | | GYPWYGWG | | | | |
| | | | SNKALPAPIEKTISKAKGQ | | LDYWGQGT | | | | |
| | | | PREPQVYTLPPSRDELTKN | | LVTVSS | | | | |
| | | | QVSLWCLVKGFYPSDIAV | | | | | | |
| | | | EWESNGQPENNYKTTPPV | | | | | | |
| | | | LDSDGSFFLYSKLTVDKSR | | | | | | |
| | | | WQQGNVFSCSVMHEALH | | | | | | |
| | | | NHYTQKSLSLSPGKGGSH | | | | | | |
| | | | HHHHH | | | | | | |
| EPI148 | RNF43 | 364 | EVQLVESGGGLVQPGGSL | 365 | EVQLVESGG | 366 | DIQMTQSPSSLS | 367 | DIQMTQ |
| | | | RLSCAASGFNIYYYSIHWV | | GLVQPGGSL | | ASVGDRVTITCR | | SPSSLSA |
| | | | RQAPGKGLEWVASIYSSS | | RLSCAASGF | | ASQSVGSALAW | | SVGDRV |
| | | | GYTSYADSVKGRFTISADT | | NIYYYSIHW | | YQQKPGKAPKL | | TITCRAS |
| | | | SKNTAYLQMNSLRAEDTA | | VRQAPGKG | | LIYSASSLYSGV | | QSVGSA |
| | | | VYYCARYPYWYFDGFDY | | LEWVASIYS | | PSRFSGSRSGTD | | LAWYQ |
| | | | WGQGTLVTVSSASTKGPS | | SSGYTSYAD | | FTLTISSLQPEDF | | QKPGKA |
| | | | VFPLAPSSKSTSGGTAALG | | SVKGRFTIS | | ATYYCQQGYSD | | PKLLIYS |
| | | | CLVKDYFPEPVTVSWNSG | | ADTSKNTA | | LITFGQGTKVEI | | ASSLYS |
| | | | ALTSGVHTFPAVLQSSGLY | | YLQMNSLR | | KRTVAAPSVFIF | | GVPSRFS |
| | | | SLSSVVTVPSSSLGTQTYI | | AEDTAVYY | | PPSDSQLKSGTA | | GSRSGT |
| | | | CNVNHKPSNTKVDKKVEPK | | CARYPYWY | | SVVCLLNNFYPR | | DFTLTIS |
| | | | SCDKTHTCPPCPAPELLGG | | FDGFDYWG | | EAKVQWKVDN | | SLQPEDF |
| | | | PSVFLFPPKPKDTLMISRT | | QGTLVTVSS | | ALQSGNSQESVT | | ATYYCQ |
| | | | PEVTCVVVDVSHEDPEVKF | | | | EQDSKDSTYSLS | | QGYSDL |
| | | | NWYVDGVEVHNAKTKPR | | | | STLTLSKADYEK | | ITFGQGT |
| | | | EEQYNSTYRVVSVLTVLH | | | | HKVYACEVTHQ | | KVEIK |
| | | | QDWLNGKEYKCKVSNKA | | | | GLSSPVTKSFNR | | |
| | | | LPAPIEKTISKAKGQPREP | | | | GEC | | |
| | | | QVYTLPPSRDELTKNQVSL | | | | | | |
| | | | WCLVKGFYPSDIAVEWES | | | | | | |
| | | | NGQPENNYKTTPPVLDSD | | | | | | |
| | | | GSFFLYSKLTVDKSRWQQ | | | | | | |
| | | | GNVFSCSVMHEALHNHYT | | | | | | |
| | | | QKSLSLSPGKGGSHHHHH | | | | | | |
| | | | H | | | | | | |
| EPI192 | HER3 | 368 | EVQLLESGGGLVQPGGSL | 369 | EVQLLESGG | 370 | DIQMTQSPSSLS | 371 | DIQMTQ |
| | | | RLSCAASGFTFSSYAMSW | | GLVQPGGSL | | ASVGDRVTITCR | | SPSSLSA |
| | | | VRQAPGKGLEWVSAINSQ | | RLSCAASGF | | ASQGISNWLAW | | SVGDRV |
| | | | GKSTYYADSVKGRFTISRD | | TFSSYAMS | | YQQKPGKAPKL | | TITCRAS |
| | | | NSKNTLYLQMNSLRAEDT | | WVRQAPGK | | LIYGASSLQSGV | | QGISNW |
| | | | AVYYCARWGDEGFDIWG | | GLEWVSAIN | | PSRFSGSGSGTD | | LAWYQ |
| | | | QGTLVTVSSASTKGPSVFP | | SQGKSTYYA | | FTLTISSLQPEDF | | QKPGKA |
| | | | LAPSSKSTSGGTAALGCLV | | DSVKGRFTI | | ATYYCQQYSSFP | | PKLLIYG |
| | | | KDYFPEPVTVSWNSGALT | | SRDNSKNTL | | TTFGQGTKVEIK | | ASSLQS |
| | | | SGVHTFPAVLQSSGLYSLS | | YLQMNSLR | | RTVAAPSVFIFPP | | GVPSRFS |
| | | | SVVTVPSSSLGTQTYICNV | | AEDTAVYY | | SDEQLKSGTASV | | GSGSGT |
| | | | NHKPSNTKVDKRVEPKSC | | CARWGDEG | | VCLLNNFYPRE | | DFTLTIS |
| | | | DKTHTCPPCPAPELLGGPS | | FDIWGQGTL | | AKVQWKVDNA | | SLQPEDF |
| | | | VFLFPPKPKDTLMISRTPE | | VTVSS | | LQSGNSQESVTE | | ATYYCQ |
| | | | VTCVVVDVSHEDPEVKFN | | | | QDSKDSTYSLSS | | QYSSFPT |
| | | | WYVDGVEVHNAKTKPRE | | | | TLTLSKADYEK | | TFGQGT |
| | | | EQYNSTYRVVSVLTVLHQ | | | | HKVYACEVTHQ | | KVEIK |
| | | | DWLNGKEYKCKVSNKAL | | | | GLSSPVTKSFNR | | |
| | | | PAPIEKTISKAKGQPREPQ | | | | GEC | | |
| | | | VYTLPPSRDELTKNQVSL | | | | | | |
| | | | WCLVKGFYPSDIAVEWES | | | | | | |
| | | | NGQPENNYKTTPPVLDSD | | | | | | |
| | | | GSFFLYSKLTVDKSRWQQ | | | | | | |
| | | | GNVFSCSVMHEALHNHYT | | | | | | |
| | | | QKSLSLSPGKGGSHHHHH | | | | | | |
| | | | H | | | | | | |
| EPI193 | HER3 | 372 | EVQLVESGGGLVQPGGSL | 373 | EVQLVESGG | 374 | DIQMTQSPSSLS | 375 | DIQMTQ |
| | | | RLSCAASGFTLSGDWIHW | | GLVQPGGSL | | ASVGDRVTITCR | | SPSSLSA |
| | | | VRQAPGKGLEWVGEISAA | | RLSCAASGF | | ASQNIATDVAW | | SVGDRV |
| | | | GGYTDYADSVKGRFTISA | | TLSGDWIH | | YQQKPGKAPKL | | TITCRAS |
| | | | DTSKNTAYLQMNSLRAED | | WVRQAPGK | | LIYSASFLYSGV | | QNIATD |
| | | | TAVYYCARESRVSFEAAM | | GLEWVGEIS | | PSRFSGSGSGTD | | NAWYQ |
| | | | DYWGQGTLVTVSSASTKG | | AAGGYTDY | | FTLTISSLQPEDF | | QKPGKA |
| | | | PSVFPLAPSSKSTSGGTAA | | ADSVKGRFT | | ATYYCQQSEPEP | | PKLLIYS |
| | | | LGCLVKDYFPEPVTVSWN | | ISADTSKNT | | YTFGQGTKVEIK | | ASFLYS |
| | | | SGALTSGVHTFPAVLQSSG | | AYLQMNSL | | RTVAAPSVFIFPP | | GVPSRFS |
| | | | LYSLSSVVTVPSSSLGTQT | | RAEDTAVY | | SDEQLKSGTASV | | GSGSGT |
| | | | YICNVNHKPSNTKVDKKV | | YCARESRVS | | VCLLNNFYPRE | | DFTLTIS |
| | | | EPKSCDKTHTCPPCPAPEL | | FEAAMDYW | | AKVQWKVDNA | | SLQPEDF |
| | | | LGGPSVFLFPPKPKDTLMI | | GQGTLVTVS | | LQSGNSQESVTE | | ATYYCQ |
| | | | SRTPEVTCVVVDVSHEDP | | S | | QDSKDSTYSLSS | | QSEPEPY |
| | | | EVKFNWYVDGVEVHNAK | | | | TLTLSKADYEK | | TFGQGT |
| | | | TKPREEQYNSTYRVVSVL | | | | HKVYACEVTHQ | | KVEIK |
| | | | TVLHQDWLNGKEYKCKV | | | | GLSSPVTKSFNR | | |
| | | | SNKALPAPIEKTISKAKGQ | | | | GEC | | |
| | | | PREPQVYTLPPSRDELTKN | | | | | | |
| | | | QVSLWCLVKGFYPSDIAV | | | | | | |
| | | | EWESNGQPENNYKTTPPV | | | | | | |
| | | | LDSDGSFFLYSKLTVDKSR | | | | | | |
| | | | WQQGNVFSCSVMHEALH | | | | | | |
| | | | NHYTQKSLSLSPGKGGSH | | | | | | |
| | | | HHHHH | | | | | | |
| EPI194 | CEA- | 376 | EVQLQESGPGLVKPGGSLS | 377 | EVQLQESGP | 378 | DIQMTQSPASLS | 379 | DIQMTQ |
| | CAM5 | | LSCAASGFVFSSYDMSWV | | GLVKPGGSL | | ASVGDRVTITCR | | SPASLSA |
| | | | RQTPERGLEWVAYISSGG | | SLSCAASGF | | ASENIFSYLAWY | | SVGDRV |
| | | | GITYAPSTVKGRFTVSRDN | | VFSSYDMS | | QQKPGKSPKLL | | TITCRAS |
| | | | AKNTLYLQMNSLTSEDTA | | WVRQTPER | | VYNTRTLAEGV | | ENIFSYL |
| | | | VYYCAAHYFGSSGPFAYW | | GLEWVAYIS | | PSRFSGSGSGTD | | AWYQQ |
| | | | GQGTLVTVSSASTKGPSVF | | SGGGITYAP | | FSLTISSLQPEDF | | KPGKSP |
| | | | PLAPSSKSTSGGTAALGCL | | STVKGRFTV | | ATYYCQHHYGT | | KLLVYN |
| | | | VKDYFPEPVTVSWNSGAL | | SRDNAKNTL | | PFTFGSGTKLEI | | TRTLAE |
| | | | TSGVHTFPAVLQSSGLYSL | | YLQMNSLTS | | KRTVAAPSVFIF | | GVPSRFS |
| | | | SSVVTVPSSSLGTQTYICN | | EDTAVYYC | | PPSDEQLKSGTA | | GSGSGT |
| | | | VNHKPSNTKVDKKVEPKS | | AAHYFGSSG | | SVVCLLNNFYPR | | DFSLTIS |
| | | | CDKTHTCPPCPAPELLGGP | | PFAYWGQG | | EAKVQWKVDN | | SLQPEDF |
| | | | SVFLFPPKPKDTLMISRTP | | TLVTVSS | | ALQSGNSQESVT | | ATYYCQ |
| | | | EVTCVVVDVSHEDPEVKFN | | | | EQDSKDSTYSLS | | HHYGTP |
| | | | WYVDGVEVHNAKTKPRE | | | | STLTLSKADYEK | | FTFGSGT |
| | | | EQYNSTYRVVSVLTVLHQ | | | | HKVYACEVTHQ | | KLEIK |
| | | | DWLNGKEYKCKVSNKAL | | | | GLSSPVTKSFNR | | |
| | | | PAPIEKTISKAKGQPREPQ | | | | GEC | | |
| | | | VYTLPPSRDELTKNQVSL | | | | | | |
| | | | WCLVKGFYPSDIAVEWES | | | | | | |
| | | | NGQPENNYKTTPPVLDSD | | | | | | |
| | | | GSFFLYSKLTVDKSRWQQ | | | | | | |
| | | | GNVFSCSVMHEALHNHYT | | | | | | |
| | | | QKSLSLSPGKGGSHHHHH | | | | | | |
| | | | H | | | | | | |
| EPI195 | F3 | 380 | EVQLLESGGGLVQPGGSL | 381 | EVQLLESGG | 382 | DIQMTQSPPSLS | 383 | DIQMTQ |
| | | | RLSCAASGFTFSNYAMSW | | GLVQPGGSL | | ASAGDRVTITCR | | SPPSLSA |
| | | | VRQAPGKGLEWVSSISGS | | RLSCAASGF | | ASQGISSRLAWY | | SAGDRV |
| | | | GDYTYYTDSVKGRFTISR | | TFSNYAMS | | QQKPEKAPKSLI | | TITCRAS |
| | | | DNSKNTLYLQMNSLRAED | | WVRQAPGK | | YAASSLQSGVPS | | QGISSRL |
| | | | TAVYYCARSPWGYYLDS | | GLEWVSSIS | | RFSGSGSGTDFT | | AWYQQ |
| | | | WGQGTLVTVSSASTKGPS | | GSGDYTYY | | LTISSLQPEDFAT | | KPEKAP |
| | | | VFPLAPSSKSTSGGTAALG | | TDSVKGRFT | | YYCQQYNSYPY | | KSLIYA |
| | | | CLVKDYFPEPVTVSWNSG | | ISRDNSKNT | | TFGQGTKLEIKR | | ASSLQS |
| | | | ALTSGVHTFPAVLQSSGLY | | LYLQMNSL | | TVAAPSVFIFPPS | | GVPSRFS |
| | | | SLSSVVTVPSSSLGTQTYI | | RAEDTAVY | | DEQLKSGTASV | | GSGSGT |
| | | | CNVNHKPSNTKVDKRVEPK | | YCARSPWG | | VCLLNNFYPRE | | DFTLTIS |
| | | | SCDKTHTCPPCPAPELLGG | | YYLDSWGQ | | AKVQWKVDNA | | SLQPEDF |
| | | | PSVFLFPPKPKDTLMISRT | | GTLVTVSS | | LQSGNSQESVTE | | ATYYCQ |
| | | | PEVTCVVVDVSHEDPEVKF | | | | QDSKDSTYSLSS | | QYNSYP |
| | | | NWYVDGVEVHNAKTKPR | | | | TLTLSKADYEK | | YTFGQG |
| | | | EEQYNSTYRVVSVLTVLH | | | | HKVYACEVTHQ | | TKLEIK |
| | | | QDWLNGKEYKCKVSNKA | | | | GLSSPVTKSFNR | | |
| | | | LPAPIEKTISKAKGQPREP | | | | GEC | | |
| | | | QVYTLPPSRDELTKNQVSL | | | | | | |
| | | | WCLVKGFYPSDIAVEWES | | | | | | |
| | | | NGQPENNYKTTPPVLDSD | | | | | | |
| | | | GSFFLYSKLTVDKSRWQQ | | | | | | |
| | | | GNVFSCSVMHEALHNHYT | | | | | | |
| | | | QKSLSLSPGKGGSHHHHH | | | | | | |
| | | | H | | | | | | |
| EPI196 | CEA- | 384 | EVQLVESGGGVVQPGRSL | 385 | EVQLVESGG | 386 | DIQLTQSPSSLSA | 387 | DIQLTQS |
| | CAM5 | | RLSCSASGFDFTTYWMSW | | GVVQPGRSL | | SVGDRVTITCKA | | PSSLSAS |
| | | | VRQAPGKGLEWIGEIHPDS | | RLSCSASGF | | SQDVGTSVAWY | | VGDRVT |
| | | | STINYAPSLKDRFTISRDN | | DFTTYWMS | | QQKPGKAPKLLI | | ITCKASQ |
| | | | AKNTLFLQMDSLRPEDTG | | WVRQAPGK | | YWTSTRHTGVP | | DVGTSV |
| | | | VYFCASLYFGFPWFAYWG | | GLEWIGEIH | | SRFSGSGSGTDF | | AWYQQ |
| | | | QGTPVTVSSASTKGPSVFP | | PDSSTINYAP | | TFTISSLQPEDIA | | KPGKAP |
| | | | LAPSSKSTSGGTAALGCLV | | SLKDRFTISR | | TYYCQQYSLYR | | KLLIYW |
| | | | KDYFPEPVTVSWNSGALT | | DNAKNTLFL | | SFGQGTKVEIKR | | TSTRHT |
| | | | SGVHTFPAVLQSSGLYSLS | | QMDSLRPED | | TVAAPSVFIFPPS | | GVPSRFS |
| | | | SVVTVPSSSLGTQTYICNV | | TGVYFCASL | | DEQLKSGTASV | | GSGSGT |
| | | | NHKPSNTKVDKRVEPKSC | | YFGFPWFAY | | VCLLNNFYPRE | | DFTFTIS |
| | | | DKTHTCPPCPAPELLGGPS | | WGQGTPVT | | AKVQWKVDNA | | SLQPEDI |
| | | | VFLFPPKPKDTLMISRTPE | | VSS | | LQSGNSQESVTE | | ATYYCQ |
| | | | VTCVVVDVSHEDPEVKFN | | | | QDSKDSTYSLSS | | QYSLYR |
| | | | WYVDGVEVHNAKTKPRE | | | | TLTLSKADYEK | | SFGQGT |
| | | | EQYNSTYRVVSVLTVLHQ | | | | HKVYACEVTHQ | | KVEIK |
| | | | DWLNGKEYKCKVSNKAL | | | | GLSSPVTKSFNR | | |
| | | | PAPIEKTISKAKGQPREPQ | | | | GEC | | |
| | | | VYTLPPSRDELTKNQVSL | | | | | | |
| | | | WCLVKGFYPSDIAVEWES | | | | | | |
| | | | NGQPENNYKTTPPVLDSD | | | | | | |
| | | | GSFFLYSKLTVDKSRWQQ | | | | | | |
| | | | GNVFSCSVMHEALHNHYT | | | | | | |
| | | | QKSLSLSPGKGGSHHHHH | | | | | | |
| | | | H | | | | | | |
| EPI197 | LY75 | 388 | EVQLVESGGGLVKPGGSL | 389 | EVQLVESGG | 390 | DVQMTQSPSSLS | 391 | DVQMT |
| | | | RLSCAASGFTFSNAWMSW | | GLVKPGGSL | | ASVGDRVTITCR | | QSPSSLS |
| | | | VRQAPGKGLEWVGRIKSK | | RLSCAASGF | | ASQSISDYLSWY | | ASVGDR |
| | | | TDGGTTDYAAPVQGRFTIS | | TFSNAWMS | | QQRPGKAPNLLI | | VTITCRA |
| | | | RDDSKNTLYLQMNSLKTE | | WVRQAPGK | | YAASNLKTGVP | | SQSISDY |
| | | | DTAVYYCTIFGVVSFDYW | | GLEWVGRIK | | SRESGSGSGTDF | | LSWYQQ |
| | | | GQGTLVTVSSASTKGPSVL | | SKTDGGTTD | | TLTISTLQPEDFA | | RPGKAP |
| | | | PLAPSSKSTSGGTAALGCL | | YAAPVQGR | | TYYCQQSYRSP | | NLLIYA |
| | | | VKDYFPEPVTVSWNSGAL | | FTISRDDSK | | WTFGQGTKVEI | | ASNLKT |
| | | | TSGVHTFPAVLQSSGLYSL | | NTLYLQMN | | KRTVAAPSVFIF | | GVPSRE |
| | | | SSVVTVPSSSLGTQTYICN | | SLKTEDTAV | | PPSDEQLKSGTA | | SGSGSG |
| | | | VNHKPSNTKVDKKVEPKS | | YYCTIFGVV | | SVVCLLNNFYPR | | TDFTLTI |
| | | | CDKTHTCPPCPAPELLGGP | | SFDYWGQG | | EAKVQWKVDN | | STLQPE |
| | | | SVFLFPPKPKDTLMISRTP | | TLVTVSS | | ALQSGNSQESVT | | DFATYY |
| | | | EVTCVVVDVSHEDPEVKFN | | | | EQDSKDSTYSLS | | CQQSYR |
| | | | WYVDGVEVHNAKTKPRE | | | | STLTLSKADYEK | | SPWTFG |
| | | | EQYNSTYRVVSVLTVLHQ | | | | HKVYACEVTHQ | | QGTKVE |
| | | | DWLNGKEYKCKVSNKAL | | | | GLSSPVTKSFNR | | IK |
| | | | PAPIEKTISKAKGQPREPQ | | | | GEC | | |
| | | | VYTLPPSRDELTKNQVSL | | | | | | |
| | | | WCLVKGFYPSDIAVEWES | | | | | | |
| | | | NGQPENNYKTTPPVLDSD | | | | | | |
| | | | GSFFLYSKLTVDKSRWQQ | | | | | | |
| | | | GNVFSCSVMHEALHNHYT | | | | | | |
| | | | QKSLSLSPGKGGSHHHHH | | | | | | |
| | | | H | | | | | | |
| EPI198 | CD45 | 394 | EVQLVESGGDRVQPGRSL | 395 | EVQLVESGG | 396 | DIQMTQSPSSVL | 397 | DIQMTQ |
| | | | TLSCVTSGFTFNNYWMTW | | DRVQPGRSL | | SASVGDRVTLSC | | SPSSVLS |
| | | | IRQVPGKGLEWVASISSSG | | TLSCVTSGF | | KASQNINKNLD | | ASVGDR |
| | | | GSIYYPDSVKGRFTISRDN | | TFNNYWMT | | WYQQKHGEAP | | VTLSCK |
| | | | AKNTLYLQMNSLRSEDTA | | WIRQVPGK | | KLLIYETNNLQT | | ASQNIN |
| | | | TYYCARDERWAGAMDA | | GLEWVASIS | | GIPSRFSGSGSGT | | KNLDW |
| | | | WGQGTSVTVSSASTKGPS | | SSGGSIYYP | | DYTLTISSLQPE | | YQQKHG |
| | | | VLPLAPSSKSTSGGTAALG | | DSVKGRFTI | | DVATYYCYQHN | | EAPKLLI |
| | | | CLVKDYFPEPVTVSWNSG | | SRDNAKNTL | | SRFTFGSGTKLEI | | YETNNL |
| | | | ALTSGVHTFPAVLQSSGLY | | YLQMNSLRS | | KRTVAAPSVFIF | | QTGIPSR |
| | | | SLSSVVTVPSSSLGTQTYI | | EDTATYYC | | PPSDEQLKSGTA | | FSGSGS |
| | | | CNVNHKPSNTKVDKKVEPK | | ARDERWAG | | SVVCLLNNFYPR | | GTDYTL |
| | | | SCDKTHTCPPCPAPELLGG | | AMDAWGQ | | EAKVQWKVDN | | TISSLQP |
| | | | PSVFLFPPKPKDTLMISRT | | GTSVTVSS | | ALQSGNSQESVT | | EDVATY |
| | | | PEVTCVVVDVSHEDPEVKF | | | | EQDSKDSTYSLS | | YCYQHN |
| | | | NWYVDGVEVHNAKTKPR | | | | STLTLSKADYEK | | SRFTFGS |
| | | | EEQYNSTYRVVSVLTVLH | | | | HKVYACEVTHQ | | GTKLEIK |
| | | | QDWLNGKEYKCKVSNKA | | | | GLSSPVTKSFNR | | |
| | | | LPAPIEKTISKAKGQPREP | | | | GEC | | |
| | | | QVYTLPPSRDELTKNQVSL | | | | | | |
| | | | WCLVKGFYPSDIAVEWES | | | | | | |
| | | | NGQPENNYKTTPPVLDSD | | | | | | |
| | | | GSFFLYSKLTVDKSRWQQ | | | | | | |
| | | | GNVFSCSVMHEALHNHYT | | | | | | |
| | | | QKSLSLSPGKGGSHHHHH | | | | | | |
| | | | H | | | | | | |
| EPI199 | SLC- | 398 | EVQLVESGGGLVQPGGSL | 399 | EVQLVESGG | 400 | DIQMTQSPSSLS | 401 | DIQMTQ |
| | 34A2 | | RLSCAASGFSFSDFAMSW | | GLVQPGGSL | | ASVGDRVTITCR | | SPSSLSA |
| | | | VRQAPGKGLEWVATIGRV | | RLSCAASGF | | SSETLVHSSGNT | | SVGDRV |
| | | | AFHTYYPDSMKGRFTISRD | | SFSDFAMSW | | YLEWYQQKPGK | | TITCRSS |
| | | | NSKNTLYLQMNSLRAEDT | | VRQAPGKG | | APKLLIYRVSNR | | ETLVHS |
| | | | AVYYCARHRGFDVGHFDF | | LEWVATIGR | | FSGVPSRFSGSG | | SGNTYL |
| | | | WGQGTLVTVSSASTKGPS | | VAFHTYYPD | | SGTDFTLTISSLQ | | EWYQQ |
| | | | VFPLAPSSKSTSGGTAALG | | SMKGRFTIS | | PEDFATYYCFQ | | KPGKAP |
| | | | CLVKDYFPEPVTVSWNSG | | RDNSKNTLY | | GSFNPLTFGQGT | | KLLIYR |
| | | | ALTSGVHTFPAVLQSSGLY | | LQMNSLRA | | KVEIKRTVAAPS | | VSNRFS |
| | | | SLSSVVTVPSSSLGTQTYI | | EDTAVYYC | | VFIFPPSDEQLKS | | GVPSRFS |
| | | | CNVNHKPSNTKVDKKVEPK | | ARHRGFDV | | GTASVVCLLNN | | GSGSGT |
| | | | SCDKTHTCPPCPAPELLGG | | GHFDFWGQ | | FYPREAKVQWK | | DFTLTIS |
| | | | PSVFLFPPKPKDTLMISRT | | GTLVTVSS | | VDNALQSGNSQ | | SLQPEDF |
| | | | PEVTCVVVDVSHEDPEVKF | | | | ESVTEQDSKDST | | ATYYCF |
| | | | NWYVDGVEVHNAKTKPR | | | | YSLSSTLTLSKA | | QGSFNP |
| | | | EEQYNSTYRVVSVLTVLH | | | | DYEKHKVYACE | | LTFGQG |
| | | | QDWLNGKEYKCKVSNKA | | | | VTHQGLSSPVTK | | TKVEIK |
| | | | LPAPIEKTISKAKGQPREP | | | | SFNRGEC | | |
| | | | QVYTLPPSRDELTKNQVSL | | | | | | |
| | | | WCLVKGFYPSDIAVEWES | | | | | | |
| | | | NGQPENNYKTTPPVLDSD | | | | | | |
| | | | GSFFLYSKLTVDKSRWQQ | | | | | | |
| | | | GNVFSCSVMHEALHNHYT | | | | | | |
| | | | QKSLSLSPGKGGSHHHHH | | | | | | |
| | | | H | | | | | | |
| EPI200 | ITGB6 | 402 | QVQLVQSGAEVKKPGASV | 403 | QVQLVQSG | 404 | DVVMTQSPLSLP | 405 | DVVMT |
| | | | KVSCKASGYSFSGYFMNW | | AEVKKPGAS | | VTLGQPASISCK | | QSPLSLP |
| | | | VRQAPGQGLEWMGLINPY | | VKVSCKAS | | SSQSLLDSDGKT | | VTLGQP |
| | | | NGDSFYNQKFKGRVTMTR | | GYSFSGYFM | | YLNWLFQRPGQ | | ASISCKS |
| | | | DTSTSTVYMELSSLRSEDT | | NWVRQAPG | | SPRRLIYLVSEL | | SQSLLDS |
| | | | AVYYCARGLRRDFDYWG | | QGLEWMGL | | DSGVPDRFSGSG | | DGKTYL |
| | | | QGTLVTVSSASTKGPSVFP | | INPYNGDSF | | SGTDFTLKISRV | | NWLFQR |
| | | | LAPSSKSTSGGTAALGCLV | | YNQKFKGR | | EAEDVGVYYC | | PGQSPR |
| | | | KDYFPEPVTVSWNSGALT | | VTMTRDTST | | WQGTHFPRTFG | | RLIYLVS |
| | | | SGVHTFPAVLQSSGLYSLS | | STVYMELSS | | GGTKLEIKRTVA | | ELDSGV |
| | | | SVVTVPSSSLGTQTYICNV | | LRSEDTAVY | | APSVFIFPPSDEQ | | PDRFSGS |
| | | | NHKPSNTKVDKKVEPKSC | | YCARGLRR | | LKSGTASVVCLL | | GSGTDF |
| | | | DKTHTCPPCPAPELLGGPS | | DFDYWGQG | | NNFYPREAKVQ | | TLKISRV |
| | | | VFLFPPKPKDTLMISRTPE | | TLVTVSS | | WKVDNALQSG | | EAEDVG |
| | | | VTCVVVDVSHEDPEVKFN | | | | NSQESVTEQDSK | | VYYCW |
| | | | WYVDGVEVHNAKTKPRE | | | | DSTYSLSSTLTL | | QGTHFP |
| | | | EQYNSTYRVVSVLTVLHQ | | | | SKADYEKHKVY | | RTFGGG |
| | | | DWLNGKEYKCKVSNKAL | | | | ACEVTHQGLSSP | | TKLEIK |
| | | | PAPIEKTISKAKGQPREPQ | | | | VTKSFNRGEC | | |
| | | | VYTLPPSRDELTKNQVSL | | | | | | |
| | | | WCLVKGFYPSDIAVEWES | | | | | | |
| | | | NGQPENNYKTTPPVLDSD | | | | | | |
| | | | GSFFLYSKLTVDKSRWQQ | | | | | | |
| | | | GNVFSCSVMHEALHNHYT | | | | | | |
| | | | QKSLSLSPGKGGSHHHHH | | | | | | |
| | | | H | | | | | | |
| EPI201 | FOLR1 | 406 | EVQLVESGGGVVQPGRSL | 407 | EVQLVESGG | 408 | DIQLTQSPSSLSA | 409 | DIQLTQS |
| | | | RLSCSASGFTFSGYGLSWV | | GVVQPGRSL | | SVGDRVTITCSV | | PSSLSAS |
| | | | RQAPGKGLEWVAMISSGG | | RLSCSASGF | | SSSISSNNLHWY | | VGDRVT |
| | | | SYTYYADSVKGRFAISRD | | TFSGYGLSW | | QQKPGKAPKPW | | ITCSVSS |
| | | | NAKNTLFLQMDSLRPEDT | | VRQAPGKG | | IYGTSNLASGVP | | SISSNNL |
| | | | GVYFCARHGDDPAWFAY | | LEWVAMISS | | SRFSGSGSGTDY | | HWYQQ |
| | | | WGQGTPVTVSSASTKGPS | | GGSYTYYA | | TFTISSLQPEDIA | | KPGKAP |
| | | | VFPLAPSSKSTSGGTAALG | | DSVKGRFAI | | TYYCQQWSSYP | | KPWIYG |
| | | | CLVKDYFPEPVTVSWNSG | | SRDNAKNTL | | YMYTFGQGTKV | | TSNLAS |
| | | | ALTSGVHTFPAVLQSSGLY | | FLQMDSLRP | | EIKRTVAAPSVFI | | GVPSRFS |
| | | | SLSSVVTVPSSSLGTQTYI | | EDTGVYFCA | | FPPSDEQLKSGT | | GSGSGT |
| | | | CNVNHKPSNTKVDKKVEPK | | RHGDDPAW | | ASVVCLLNNFY | | DYTFTIS |
| | | | SCDKTHTCPPCPAPELLGG | | FAYWGQGT | | PREAKVQWKVD | | SLQPEDI |
| | | | PSVFLFPPKPKDTLMISRT | | PVTVSS | | NALQSGNSQES | | ATYYCQ |
| | | | PEVTCVVVDVSHEDPEVKF | | | | VTEQDSKDSTYS | | QWSSYP |
| | | | NWYVDGVEVHNAKTKPR | | | | LSSTLTLSKADY | | YMYTFG |
| | | | EEQYNSTYRVVSVLTVLH | | | | EKHKVYACEVT | | QGTKVE |
| | | | QDWLNGKEYKCKVSNKA | | | | HQGLSSPVTKSF | | IK |
| | | | LPAPIEKTISKAKGQPREP | | | | NRGEC | | |
| | | | QVYTLPPSRDELTKNQVSL | | | | | | |
| | | | WCLVKGFYPSDIAVEWES | | | | | | |
| | | | NGQPENNYKTTPPVLDSD | | | | | | |
| | | | GSFFLYSKLTVDKSRWQQ | | | | | | |
| | | | GNVFSCSVMHEALHNHYT | | | | | | |
| | | | QKSLSLSPGKGGSHHHHH | | | | | | |
| | | | H | | | | | | |
| EPI202 | FOLR1 | 410 | QVQLVQSGAEVVKPGASV | 411 | QVQLVQSG | 412 | DIVLTQSPLSLA | 413 | DIVLTQS |
| | | | KISCKASGYTFTGYFMNW | | AEVVKPGAS | | VSLGQPAIISCK | | PLSLAVS |
| | | | VKQSPGQSLEWIGRIHPYD | | VKISCKASG | | ASQSVSFAGTSL | | LGQPAII |
| | | | GDTFYNQKFQGKATLTVD | | YTFTGYFM | | MHWYHQKPGQ | | SCKASQ |
| | | | KSSNTAHMELLSLTSEDFA | | NWVKQSPG | | QPRLLIYRASNL | | SVSFAG |
| | | | VYYCTRYDGSRAMDYWG | | QSLEWIGRI | | EAGVPDRFSGSG | | TSLMHW |
| | | | QGTTVTVSSASTKGPSVFP | | HPYDGDTFY | | SKTDFTLTISPVE | | YHQKPG |
| | | | LAPSSKSTSGGTAALGCLV | | NQKFQGKA | | AEDAATYYCQQ | | QQPRLLI |
| | | | KDYFPEPVTVSWNSGALT | | TLTVDKSSN | | SREYPYTFGGGT | | YRASNL |
| | | | SGVHTFPAVLQSSGLYSLS | | TAHMELLSL | | KLEIKRTVAAPS | | EAGVPD |
| | | | SVVTVPSSSLGTQTYICNV | | TSEDFAVYY | | VFIFPPSDEQLKS | | RFSGSGS |
| | | | NHKPSNTKVDKKVEPKSC | | CTRYDGSRA | | GTASVVCLLNN | | KTDFTL |
| | | | DKTHTCPPCPAPELLGGPS | | MDYWGQGT | | FYPREAKVQWK | | TISPVEA |
| | | | VFLFPPKPKDTLMISRTPE | | TVTVSS | | VDNALQSGNSQ | | EDAATY |
| | | | VTCVVVDVSHEDPEVKFN | | | | ESVTEQDSKDST | | YCQQSR |
| | | | WYVDGVEVHNAKTKPRE | | | | YSLSSTLTLSKA | | EYPYTF |
| | | | EQYNSTYRVVSVLTVLHQ | | | | DYEKHKVYACE | | GGGTKL |
| | | | DWLNGKEYKCKVSNKAL | | | | VTHQGLSSPVTK | | EIK |
| | | | PAPIEKTISKAKGQPREPQ | | | | SFNRGEC | | |
| | | | VYTLPPSRDELTKNQVSL | | | | | | |
| | | | WCLVKGFYPSDIAVEWES | | | | | | |
| | | | NGQPENNYKTTPPVLDSD | | | | | | |
| | | | GSFFLYSKLTVDKSRWQQ | | | | | | |
| | | | GNVFSCSVMHEALHNHYT | | | | | | |
| | | | QKSLSLSPGKGGSHHHHH | | | | | | |
| | | | H | | | | | | |
| EPI203 | MUC16 | 414 | QVQLVESGGGLVKPGGSL | 415 | QVQLVESG | 416 | DIQMTQSPSSLS | 417 | DIQMTQ |
| | | | RLSCAASGFTFSNYYMSW | | GGLVKPGGS | | ASVGDRVTITCR | | SPSSLSA |
| | | | VRQAPGKGLEWISYISGRG | | LRLSCAASG | | ASQSISTYLNWY | | SVGDRV |
| | | | STIFYADSVKGRITISRDN | | FTFSNYYMS | | QQKPGKAPKLLI | | TITCRAS |
| | | | AKNSLFLQMNSLRAEDTAV | | WVRQAPGK | | YTASSLQSGVPS | | QSISTYL |
| | | | YFCVKDRGGYSPYWGQG | | GLEWISYIS | | RFSGSGSGTDFT | | NWYQQ |
| | | | TLVTVSSASTKGPSVLPLA | | GRGSTIFYA | | LTISSLQPEDFAT | | KPGKAP |
| | | | PSSKSTSGGTAALGCLVK | | DSVKGRITIS | | YYCQQSYSTPPI | | KLLIYTA |
| | | | DYFPEPVTVSWNSGALTS | | RDNAKNSLF | | TFGQGTRLEIKR | | SSLQSG |
| | | | GVHTFPAVLQSSGLYSLSS | | LQMNSLRA | | TVAAPSVFIFPPS | | VPSRFSG |
| | | | VVTVPSSSLGTQTYICNVN | | EDTAVYFCV | | DEQLKSGTASV | | SGSGTD |
| | | | HKPSNTKVDKKVEPKSCD | | KDRGGYSP | | VCLLNNFYPRE | | FTLTISS |
| | | | KTHTCPPCPAPELLGGPSV | | YWGQGTLV | | AKVQWKVDNA | | LQPEDF |
| | | | FLFPPKPKDTLMISRTPEV | | TVSS | | LQSGNSQESVTE | | ATYYCQ |
| | | | TCVVVDVSHEDPEVKFNW | | | | QDSKDSTYSLSS | | QSYSTPP |
| | | | YVDGVEVHNAKTKPREEQ | | | | TLTLSKADYEK | | ITFGQGT |
| | | | YNSTYRVVSVLTVLHQD | | | | HKVYACEVTHQ | | RLEIK |
| | | | WLNGKEYKCKVSNKALP | | | | GLSSPVTKSFNR | | |
| | | | APIEKTISKAKGQPREPQV | | | | GEC | | |
| | | | YTLPPSRDELTKNQVSLW | | | | | | |
| | | | CLVKGFYPSDIAVEWESN | | | | | | |
| | | | GQPENNYKTTPPVLDSDG | | | | | | |
| | | | SFFLYSKLTVDKSRWQQG | | | | | | |
| | | | NVFSCSVMHEALHNHYTQ | | | | | | |
| | | | KSLSLSPGKGGSHHHHHH | | | | | | |
| EPI239 | RNF43 | 418 | EVQLVESGGGLVQPGGSL | 419 | EVQLVESGG | 420 | DIQMTQSPSSLS | 421 | DIQMTQ |
| | | | RLSCVVSGFTFSYYDMHW | | GLVQPGGSL | | ASVGDRVTITCR | | SPSSLSA |
| | | | VRQVTGKGLEWVSAIGTA | | RLSCVVSGF | | ASQSISSYLNWY | | SVGDRV |
| | | | GATYYPGSVKGRFTISREN | | TFSYYDMH | | QQKPGKAPKLLI | | TITCRAS |
| | | | AKNSLYLQMNSLRAGDTA | | WVRQVTGK | | YAASSLQSGVPS | | QSISSYL |
| | | | VYYCARDRGYSGYDAYY | | GLEWVSAIG | | RFSGSGSGTDFT | | NWYQQ |
| | | | FDFWGQGTLVTVSSASTK | | TAGATYYP | | LTISSLQPEDFAT | | KPGKAP |
| | | | GPSVFPLAPSSKSTSGGTA | | GSVKGRFTI | | YYCQQSYSTPPT | | KLLIYA |
| | | | ALGCLVKDYFPEPVTVSW | | SRENAKNSL | | FGQGTKVEIKRT | | ASSLQS |
| | | | NSGALTSGVHTFPAVLQSS | | YLQMNSLR | | VAAPSVEIFPPS | | GVPSRFS |
| | | | GLYSLSSVVTVPSSSLGTQ | | AGDTAVYY | | DEQLKSGTASV | | GSGSGT |
| | | | TYICNVNHKPSNTKVDKK | | CARDRGYS | | VCLLNNFYPRE | | DFTLTIS |
| | | | VEPKSCDKTHTCPPCPAPE | | GYDAYYFD | | AKVQWKVDNA | | SLQPEDF |
| | | | LLGGPSVFLFPPKPKDTLM | | FWGQGTLV | | LQSGNSQESVTE | | ATYYCQ |
| | | | ISRTPEVTCVVVDVSHEDP | | TVSS | | QDSKDSTYSLSS | | QSYSTPP |
| | | | EVKFNWYVDGVEVHNAK | | | | TLTLSKADYEK | | TFGQGT |
| | | | TKPREEQYNSTYRVVSVL | | | | HKVYACEVTHQ | | KVEIK |
| | | | TVLHQDWLNGKEYKCKV | | | | GLSSPVTKSFNR | | |
| | | | SNKALPAPIEKTISKAKGQ | | | | GEC | | |
| | | | PREPQVYTLPPSRDELTKN | | | | | | |
| | | | QVSLWCLVKGFYPSDIAV | | | | | | |
| | | | EWESNGQPENNYKTTPPV | | | | | | |
| | | | LDSDGSFFLYSKLTVDKSR | | | | | | |
| | | | WQQGNVFSCSVMHEALH | | | | | | |
| | | | NHYTQKSLSLSPGKGGSH | | | | | | |
| | | | HHHHH | | | | | | |
| EPI240 | RNF43 | 422 | QVQLQESGPGLVKPSETLS | 423 | QVQLQESGP | 424 | DIQMTQSPSSLS | 425 | DIQMTQ |
| | | | LTCTVSGGSISSSNYYWG | | GLVKPSETL | | ASVGDRVTITCR | | SPSSLSA |
| | | | WIRQPPGKGLEWIGNIYYR | | SLTCTVSGG | | ASQSISSYLNWY | | SVGDRV |
| | | | GYTYYNPSLKSRVTISVDT | | SISSSNYYW | | QQKPGKAPKLLI | | TITCRAS |
| | | | SKKQFSLTLSSVTAADTA | | GWIRQPPGK | | YAASSLQSGVPS | | QSISSYL |
| | | | MYYCAREGSDYGDYVGA | | GLEWIGNIY | | RFSGSGSGTDFT | | NWYQQ |
| | | | FDIWDQGTMVTVSSASTK | | YRGYTYYN | | LTISSLQPEDFAT | | KPGKAP |
| | | | GPSVFPLAPSSKSTSGGTA | | PSLKSRVTIS | | YYCQQSYSTPPT | | KLLIYA |
| | | | ALGCLVKDYFPEPVTVSW | | VDTSKKQFS | | FGQGTKVEIKRT | | ASSLQS |
| | | | NSGALTSGVHTFPAVLQSS | | LTLSSVTAA | | VAAPSVEIFPPS | | GVPSRFS |
| | | | GLYSLSSVVTVPSSSLGTQ | | DTAMYYCA | | DEQLKSGTASV | | GSGSGT |
| | | | TYICNVNHKPSNTKVDKK | | REGSDYGD | | VCLLNNFYPRE | | DFTLTIS |
| | | | VEPKSCDKTHTCPPCPAPE | | YVGAFDIW | | AKVQWKVDNA | | SLQPEDF |
| | | | LLGGPSVFLFPPKPKDTLM | | DQGTMVTV | | LQSGNSQESVTE | | ATYYCQ |
| | | | ISRTPEVTCVVVDVSHEDP | | SS | | QDSKDSTYSLSS | | QSYSTPP |
| | | | EVKFNWYVDGVEVHNAK | | | | TLTLSKADYEK | | TFGQGT |
| | | | TKPREEQYNSTYRVVSVL | | | | HKVYACEVTHQ | | KVEIK |
| | | | TVLHQDWLNGKEYKCKV | | | | GLSSPVTKSFNR | | |
| | | | SNKALPAPIEKTISKAKGQ | | | | GEC | | |
| | | | PREPQVYTLPPSRDELTKN | | | | | | |
| | | | QVSLWCLVKGFYPSDIAV | | | | | | |
| | | | EWESNGQPENNYKTTPPV | | | | | | |
| | | | LDSDGSFFLYSKLTVDKSR | | | | | | |
| | | | WQQGNVFSCSVMHEALH | | | | | | |
| | | | NHYTQKSLSLSPGKGGSH | | | | | | |
| | | | HHHHH | | | | | | |
| EPI241 | RNF43 | 426 | EVQLVQSGGGLVQPGGSL | 427 | EVQLVQSG | 428 | DIQMTQSPSSLS | 429 | DIQMTQ |
| | | | RLSCAASGFTFSYYDMHW | | GGLVQPGGS | | ASVGDRVTITCR | | SPSSLSA |
| | | | VRQVTGKGLEWVSTIGAT | | LRLSCAASG | | ASQSISSYLNWY | | SVGDRV |
| | | | GDTYYSDSVKGRFTISRQN | | FTFSYYDMH | | QQKPGKAPKLLI | | TITCRAS |
| | | | AKNSLYLQINSLRAGDTA | | WVRQVTGK | | YAASSLQSGVPS | | QSISSYL |
| | | | VYYCVRDRGYIGYDSYYF | | GLEWVSTIG | | RFSGSGSGTDFT | | NWYQQ |
| | | | DNWGQGTLVTVSSASTKG | | ATGDTYYS | | LTISSLQPEDFAT | | KPGKAP |
| | | | PSVFPLAPSSKSTSGGTAA | | DSVKGRFTI | | YYCQQSYSTPPT | | KLLIYA |
| | | | LGCLVKDYFPEPVTVSWN | | SRQNAKNSL | | FGQGTKVEIKRT | | ASSLQS |
| | | | SGALTSGVHTFPAVLQSSG | | YLQINSLRA | | VAAPSVEIFPPS | | GVPSRFS |
| | | | LYSLSSVVTVPSSSLGTQT | | GDTAVYYC | | DEQLKSGTASV | | GSGSGT |
| | | | YICNVNHKPSNTKVDKKV | | VRDRGYIGY | | VCLLNNFYPRE | | DFTLTIS |
| | | | EPKSCDKTHTCPPCPAPEL | | DSYYFDNW | | AKVQWKVDNA | | SLQPEDF |
| | | | LGGPSVFLFPPKPKDTLMI | | GQGTLVTVS | | LQSGNSQESVTE | | ATYYCQ |
| | | | SRTPEVTCVVVDVSHEDP | | S | | QDSKDSTYSLSS | | QSYSTPP |
| | | | EVKFNWYVDGVEVHNAK | | | | TLTLSKADYEK | | TFGQGT |
| | | | TKPREEQYNSTYRVVSVL | | | | HKVYACEVTHQ | | KVEIK |
| | | | TVLHQDWLNGKEYKCKV | | | | GLSSPVTKSFNR | | |
| | | | SNKALPAPIEKTISKAKGQ | | | | GEC | | |
| | | | PREPQVYTLPPSRDELTKN | | | | | | |
| | | | QVSLWCLVKGFYPSDIAV | | | | | | |
| | | | EWESNGQPENNYKTTPPV | | | | | | |
| | | | LDSDGSFFLYSKLTVDKSR | | | | | | |
| | | | WQQGNVFSCSVMHEALH | | | | | | |
| | | | NHYTQKSLSLSPGKGGSH | | | | | | |
| | | | HHHHH | | | | | | |
| EPI242 | RNF43 | 430 | EVQLVQSGAEVKKPGASV | 431 | EVQLVQSG | 432 | EIVMTQSPATLS | 433 | EIVMTQ |
| | | | KVSCKASGYTFTTYTIHW | | AEVKKPGAS | | VSPGERATLSCK | | SPATLSV |
| | | | VRQAPGQGLEWMGYINPR | | VKVSCKAS | | ASQNVGINVAW | | SPGERA |
| | | | SGYTEYNQKFQDRVTMTR | | GYTFTTYTI | | YQQKPGQAPRA | | TLSCKA |
| | | | DTSTSTVYMELSSLRSEDT | | HWVRQAPG | | LIYSASYRYSGIP | | SQNVGI |
| | | | AVYYCARSYEFWGQGTT | | QGLEWMGY | | ARFSGSGSGTEF | | NVAWY |
| | | | VTVSSAKTTAPSVYPLAPV | | INPRSGYTE | | TLTISSLQSEDFA | | QQKPGQ |
| | | | CGDTTGSSVTLGCLVKGY | | YNQKFQDR | | VYYCHQYKTYP | | APRALIY |
| | | | FPEPVTLTWNSGSLSSGVH | | VTMTRDTST | | YTFGGGTKLEIK | | SASYRY |
| | | | TFPAVLQSDLYTLSSSVTV | | STVYMELSS | | RADAAPTVSIFP | | SGIPARF |
| | | | TSSTWPSQSITCNVAHPAS | | LRSEDTAVY | | PSSEQLTSGGAS | | SGSGSG |
| | | | STKVDKKIEPKSCDKTHTC | | YCARSYEF | | VVCFLNNFYPK | | TEFTLTI |
| | | | PPCPAPELLGGPSVFLFPP | | WGQGTTVT | | DINVKWKIDGSE | | SSLQSED |
| | | | KPKDTLMISRTPEVTCVVV | | VSS | | RQNGVLNSWTD | | FAVYYC |
| | | | DVSHEDPEVKFNWYVDGV | | | | QDSKDSTYSMS | | HQYKTY |
| | | | EVHNAKTKPREEQYNSTY | | | | STLTLTKDEYER | | PYTFGG |
| | | | RVVSVLTVLHQDWLNGK | | | | HNSYTCEATHK | | GTKLEIK |
| | | | EYKCKVSNKALPAPIEKTI | | | | TSTSPIVKSFNR | | |
| | | | SKAKGQPREPQVYTLPPSR | | | | NEC | | |
| | | | DELTKNQVSLWCLVKGFY | | | | | | |
| | | | PSDIAVEWESNGQPENNY | | | | | | |
| | | | KTTPPVLDSDGSFFLYSKL | | | | | | |
| | | | TVDKSRWQQGNVFSCSV | | | | | | |
| | | | MHEALHNHYTQKSLSLSP | | | | | | |
| | | | GKGGSHHHHHH | | | | | | |
| EPI243 | RNF43 | 434 | AVQLVESGGGSVQPGRSM | 435 | AVQLVESG | 436 | DVVLTQTPVSLS | 437 | DVVLTQ |
| | | | RLSCAASGFTFSNYDMTW | | GGSVQPGRS | | VTVGDQASISCR | | TPVSLSV |
| | | | VRQAPTKGLEWVASITSD | | MRLSCAAS | | SSQSLEYSDGYS | | TVGDQA |
| | | | GGSTYSRDSVKGRFTISRD | | GFTFSNYDM | | YLEWYLQKPGQ | | SISCRSS |
| | | | NAKSTLYLQMDSLRSEDT | | TWVRQAPT | | SPQLLIYEVSSRF | | QSLEYS |
| | | | ATYYCTTDRGRYLPYYFD | | KGLEWVASI | | SGVPDRFIGSGS | | DGYSYL |
| | | | YWGQGVMVTVSAAKTTA | | TSDGGSTYS | | GTDFTLKISRVE | | EWYLQK |
| | | | PSVYPLAPVCGDTTGSSVT | | RDSVKGRFT | | PEDLGVYYCFQ | | PGQSPQ |
| | | | LGCLVKGYFPEPVTLTWN | | ISRDNAKST | | AIHDPTFGAGTK | | LLIYEVS |
| | | | SGSLSSGVHTFPAVLQSDL | | LYLQMDSL | | LELKRADAAPT | | SRFSGVP |
| | | | YTLSSSVTVTSSTWPSQSI | | RSEDTATYY | | VSIFPPSSEQLTS | | DRFIGSG |
| | | | TCNVAHPASSTKVDKKIEP | | CTTDRGRYL | | GGASVVCFLNN | | SGTDFT |
| | | | KSCDKTHTCPPCPAPELLG | | PYYFDYWG | | FYPKDINVKWKI | | KISRVE |
| | | | GPSVFLFPPKPKDTLMISR | | QGVMVTVS | | DGSERQNGVLN | | PEDLGV |
| | | | TPEVTCVVVDVSHEDPEV | | A | | SWTDQDSKDST | | YYCFQA |
| | | | KFNWYVDGVEVHNAKTK | | | | YSMSSTLTLTKD | | IHDPTFG |
| | | | PREEQYNSTYRVVSVLTV | | | | EYERHNSYTCE | | AGTKLE |
| | | | LHQDWLNGKEYKCKVSN | | | | ATHKTSTSPIVK | | ILK |
| | | | KALPAPIEKTISKAKGQPR | | | | SFNRNEC | | |
| | | | EPQVYTLPPSRDELTKNQV | | | | | | |
| | | | SLWCLVKGFYPSDIAVEW | | | | | | |
| | | | ESNGQPENNYKTTPPVLDS | | | | | | |
| | | | DGSFFLYSKLTVDKSRWQ | | | | | | |
| | | | QGNVFSCSVMHEALHNH | | | | | | |
| | | | YTQKSLSLSPGKGGSHHH | | | | | | |
| | | | HHH | | | | | | |
| EPI244 | RNF43 | 438 | QVQLKESGPGLVQPSQTLS | 439 | QVQLKESGP | 440 | DTVLTQSPALA | 441 | DTVLTQ |
| | | | LTCTVSGFSLTTYSVHWV | | GLVQPSQTL | | VSPGERVTISCR | | SPALAV |
| | | | RQHSGKNLEWMGRMWT | | SLTCTVSGF | | ASESVSKLMHW | | SPGERV |
| | | | AGDTSYNSAFTSRLNIFRD | | SLTTYSVHW | | YQQRPGQQPQL | | TISCRAS |
| | | | TSKSQVFLKMNSLQTEDT | | VRQHSGKN | | LIYLTSHLASGV | | ESVSKL |
| | | | GTYYCARSSYTSGYPFDS | | LEWMGRM | | PARFSGSGSGTD | | MHWYQ |
| | | | WGQGVMVTVSSAKTTAP | | WTAGDTSY | | FTLTIDPVEADD | | QRPGQQ |
| | | | SVYPLAPVCGDTTGSSVTL | | NSAFTSRLNI | | TATYYCQQSRN | | PQLLIYL |
| | | | GCLVKGYFPEPVTLTWNS | | FRDTSKSQV | | DPTFGAGTKLEL | | TSHLAS |
| | | | GSLSSGVHTFPAVLQSDLY | | FLKMNSLQT | | KRADAAPTVSIF | | GVPARF |
| | | | TLSSSVTVTSSTWPSQSIT | | EDTGTYYC | | PPSSEQLTSGGA | | SGSGSG |
| | | | CNVAHPASSTKVDKKIEPK | | ARSSYTSGY | | SVVCFLNNFYPK | | TDFTLTI |
| | | | SCDKTHTCPPCPAPELLGG | | PFDSWGQG | | DINVKWKIDGSE | | DPVEAD |
| | | | PSVFLFPPKPKDTLMISRT | | VMVTVSS | | RQNGVLNSWTD | | DTATYY |
| | | | PEVTCVVVDVSHEDPEVKF | | | | QDSKDSTYSMS | | CQQSRN |
| | | | NWYVDGVEVHNAKTKPRE | | | | STLTLTKDEYER | | DPTFGA |
| | | | EQYNSTYRVVSVLTVLHQ | | | | HNSYTCEATHK | | GTKLEL |
| | | | DWLNGKEYKCKVSNKAL | | | | TSTSPIVKSFNR | | K |
| | | | PAPIEKTISKAKGQPREPQ | | | | NEC | | |
| | | | VYTLPPSRDELTKNQVSL | | | | | | |
| | | | WCLVKGFYPSDIAVEWES | | | | | | |
| | | | NGQPENNYKTTPPVLDSD | | | | | | |
| | | | GSFFLYSKLTVDKSRWQQ | | | | | | |
| | | | GNVFSCSVMHEALHNHYT | | | | | | |
| | | | QKSLSLSPGKGGSHHHHH | | | | | | |
| | | | H | | | | | | |
| EPI245 | RNF43 | 442 | EVQLVESGGGLVPPGKSL | 443 | EVQLVESGG | 444 | DIVMTQSPFSLA | 445 | DIVMTQ |
| | | | KLSCSASGFPFSNYGMHW | | GLVPPGKSL | | VSEGDMVTIMC | | SPFSLAV |
| | | | IRQAPGKGLDWVGYISSNS | | KLSCSASGF | | RSSQSLLSSGNQ | | SEGDMV |
| | | | GTIYADAVKGRFTISRDNA | | PFSNYGMH | | KNYLAWYQQK | | TIMCRSS |
| | | | KNTLYLLINSLKSEDTAM | | WIRQAPGK | | PGQSPKLLIYHA | | QSLLSSG |
| | | | YYCARGYFDGYYRFWGQ | | GLDWVGYIS | | STRQSGVPDRFI | | NQKNYL |
| | | | GVMVTVSSAKTTAPSVYP | | SNSGTIYAD | | GSGSGTDFTLTI | | AWYQQ |
| | | | LAPVCGDTTGSSVTLGCL | | AVKGRFTIS | | SDVQAEDLADY | | KPGQSP |
| | | | VKGYFPEPVTLTWNSGSL | | RDNAKNTL | | YCLQHYSSPTFG | | KLLIYH |
| | | | SSGVHTFPAVLQSDLYTLS | | YLLINSLKS | | SGTKLEIKRADA | | ASTRQS |
| | | | SSVTVTSSTWPSQSITCNV | | EDTAMYYCA | | APTVSIFPPSSEQ | | GVPDRFI |
| | | | AHPASSTKVDKKIEPKSCD | | RGYFDGYY | | LTSGGASVVCFL | | GSGSGT |
| | | | KTHTCPPCPAPELLGGPSV | | RFWGQGVM | | NNFYPKDINVK | | DFTLTIS |
| | | | FLFPPKPKDTLMISRTPEV | | VTVSS | | WKIDGSERQNG | | DVQAED |
| | | | TCVVVDVSHEDPEVKFNW | | | | VLNSWTDQDSK | | LADYYC |
| | | | YVDGVEVHNAKTKPREEQ | | | | DSTYSMSSTLTL | | LQHYSS |
| | | | YNSTYRVVSVLTVLHQD | | | | TKDEYERHNSY | | PTFGSGT |
| | | | WLNGKEYKCKVSNKALP | | | | TCEATHKTSTSP | | KLEIK |
| | | | APIEKTISKAKGQPREPQV | | | | IVKSFNRNEC | | |
| | | | YTLPPSRDELTKNQVSLW | | | | | | |
| | | | CLVKGFYPSDIAVEWESN | | | | | | |
| | | | GQPENNYKTTPPVLDSDG | | | | | | |
| | | | SFFLYSKLTVDKSRWQQG | | | | | | |
| | | | NVFSCSVMHEALHNHYTQ | | | | | | |
| | | | KSLSLSPGKGGSHHHHHH | | | | | | |
| EPI246 | RNF43 | 446 | EVHLVESGGGLVQPGGSL | 447 | EVHLVESGG | 448 | DTVLTQSPALTV | 449 | DTVLTQ |
| | | | KLSCAASGFTFSNYDMAW | | GLVQPGGSL | | SPGDKITISCRAS | | SPALTVS |
| | | | VRQAPTRGLEWVASISPG | | KLSCAASGF | | EGVNTRIHWYQ | | PGDKITI |
| | | | GGKTYYRDSVKGRLTISR | | TFSNYDMA | | QKSGQQPKLLIY | | SCRASE |
| | | | NNAENTQYLQIDSLRSEDT | | WVRQAPTR | | GASNLDSGVPD | | GVNTRI |
| | | | ATYYCSRLGPAYSGEWFA | | GLEWVASIS | | RFSGSGFGTDFT | | HWYQQ |
| | | | YWGQGTLVTVSSAKTTAP | | PGGGKTYY | | LTIDPVEASDTA | | KSGQQP |
| | | | SVYPLAPVCGDTTGSSVTL | | RDSVKGRLT | | TYFCQQSWNVP | | KLLIYG |
| | | | GCLVKGYFPEPVTLTWNS | | ISRNNAENT | | HTFGGGTKLEL | | ASNLDS |
| | | | GSLSSGVHTFPAVLQSDLY | | QYLQIDSLR | | KRADAAPTVSIF | | GVPDRF |
| | | | TLSSSVTVTSSTWPSQSIT | | SEDTATYYC | | PPSSEQLTSGGA | | SGSGFG |
| | | | CNVAHPASSTKVDKKIEPK | | SRLGPAYSG | | SVVCFLNNFYPK | | TDFTLTI |
| | | | SCDKTHTCPPCPAPELLGG | | EWFAYWGQ | | DINVKWKIDGSE | | DPVEAS |
| | | | PSVFLFPPKPKDTLMISRT | | GTLVTVSS | | RQNGVLNSWTD | | DTATYF |
| | | | PEVTCVVVDVSHEDPEVKF | | | | QDSKDSTYSMS | | CQQSWN |
| | | | NWYVDGVEVHNAKTKPRE | | | | STLTLTKDEYER | | NVPHTFG |
| | | | EQYNSTYRVVSVLTVLHQ | | | | HNSYTCEATHK | | GGTKLE |
| | | | DWLNGKEYKCKVSNKAL | | | | TSTSPIVKSFNR | | LK |
| | | | PAPIEKTISKAKGQPREPQ | | | | NEC | | |
| | | | VYTLPPSRDELTKNQVSL | | | | | | |
| | | | WCLVKGFYPSDIAVEWES | | | | | | |
| | | | NGQPENNYKTTPPVLDSD | | | | | | |
| | | | GSFFLYSKLTVDKSRWQQ | | | | | | |
| | | | GNVFSCSVMHEALHNHYT | | | | | | |
| | | | QKSLSLSPGKGGSHHHHH | | | | | | |
| | | | H | | | | | | |
| EPI247 | RNF43 | 450 | EVQLAESGGGLEQPGRSL | 451 | EVQLAESGG | 452 | DIVLTQSPALAV | 453 | DIVLTQS |
| | | | KLSCAASGFTFSNYDMAW | | GLEQPGRSL | | SLGQRATISCRA | | PALAVS |
| | | | VRQAPTKGLEWVASIIKSG | | KLSCAASGF | | SQSVTISGFNLM | | LGQRAT |
| | | | DTSYYRDSVKGRFTVSRD | | TFSNYDMA | | HWYQQKPGQQP | | ISCRASQ |
| | | | NAKSTLYLQMDSLRSEDT | | WVRQAPTK | | KLLIYRASNLAF | | SVTISGF |
| | | | ATYYCARHGVGSYDWFA | | GLEWVASII | | GIPARFSGSGSG | | NLMHW |
| | | | DWGQGTLVTVSSAKTTAP | | KSGDTSYYR | | TDFTLTINPVQA | | YQQKPG |
| | | | SVYPLAPVCGDTTGSSVTL | | DSVKGRFTV | | DDFTTYYCQQS | | QQPKLLI |
| | | | GCLVKGYFPEPVTLTWNS | | SRDNAKSTL | | RKSRTFGGGTK | | YRASNL |
| | | | GSLSSGVHTFPAVLQSDLY | | YLQMDSLRS | | LELKRADAAPT | | AFGIPAR |
| | | | TLSSSVTVTSSTWPSQSIT | | EDTATYYC | | VSIFPPSSEQLTS | | FSGSGS |
| | | | CNVAHPASSTKVDKKIEPKS | | ARHGVGSY | | GGASVVCFLNN | | GTDFTL |
| | | | CDKTHTCPPCPAPELLGGP | | DWFADWGQ | | FYPKDINVKWKI | | TINPVQ |
| | | | SVFLFPPKPKDTLMISRTP | | GTLVTVSS | | DGSERQNGVLN | | ADDFTT |
| | | | EVTCVVVDVSHEDPEVKFN | | | | YSMSSTLTLTKD | | RKSRTF |
| | | | WYVDGVEVHNAKTKPRE | | | | EYERHNSYTCE | | GGGTKL |
| | | | EQYNSTYRVVSVLTVLHQ | | | | ATHKTSTSPIVK | | ELK |
| | | | DWLNGKEYKCKVSNKAL | | | | SFNRNEC | | |
| | | | PAPIEKTISKAKGQPREPQ | | | | | | |
| | | | VYTLPPSRDELTKNQVSL | | | | | | |
| | | | WCLVKGFYPSDIAVEWES | | | | | | |
| | | | NGQPENNYKTTPPVLDSD | | | | | | |
| | | | GSFFLYSKLTVDKSRWQQ | | | | | | |
| | | | GNVFSCSVMHEALHNHYT | | | | | | |
| | | | QKSLSLSPGKGGSHHHHH | | | | | | |
| EPI248 | HER3 | 454 | QVQLQQWGAGLLKPSETL | 455 | QVQLQQWG | 456 | DIEMTQSPDSLA | 457 | DIEMTQ |
| | | | SLTCAVYGGSFSGYYWSW | | AGLLKPSET | | VSLGERATINCR | | SPDSLA |
| | | | IRQPPGKGLEWIGEINHSG | | LSLTCAVYG | | SSQSVLYSSSNR | | VSLGER |
| | | | STNYNPSLKSRVTISVETS | | GSFSGYYWS | | NYLAWYQQNP | | ATINCRS |
| | | | KNQFSLKLSSVTAADTAV | | WIRQPPGKG | | GQPPKLLIYWAS | | SQSVLY |
| | | | YYCARDKWTWYFDLWG | | LEWIGEINH | | TRESGVPDRFSG | | SSSNRN |
| | | | RGTLVTVSSASTKGPSVFP | | SGSTNYNPS | | SGSGTDFTLTISS | | YLAWY |
| | | | LAPSSKSTSGGTAALGCLV | | LKSRVTISV | | LQAEDVAVYYC | | QQNPGQ |
| | | | KDYFPEPVTVSWNSGALT | | ETSKNQFSL | | QQYYSTPRTFG | | PPKLLIY |
| | | | SGVHTFPAVLQSSGLYSLS | | KLSSVTAAD | | QGTKVEIKRTV | | WASTRE |
| | | | SVVTVPSSSLGTQTYICNV | | TAVYYCAR | | AAPSVFIFPPSDE | | SGVPDR |
| | | | NHKPSNTKVDKRVEPKSC | | DKWTWYFD | | QLKSGTASVVC | | FSGSGS |
| | | | DKTHTCPPCPAPELLGGPS | | LWGRGTLV | | LLNNFYPREAK | | GTDFTL |
| | | | VFLFPPKPKDTLMISRTPE | | TVSS | | VQWKVDNALQ | | TISSLQA |
| | | | VTCVVVDVSHEDPEVKFN | | | | SGNSQESVTEQD | | EDVAVY |
| | | | WYVDGVEVHNAKTKPRE | | | | SKDSTYSLSSTL | | YCQQYY |
| | | | EQYNSTYRVVSVLTVLHQ | | | | TLSKADYEKHK | | STPRTFG |
| | | | DWLNGKEYKCKVSNKAL | | | | VYACEVTHQGL | | QGTKVE |
| | | | PAPIEKTISKAKGQPREPQ | | | | SSPVTKSFNRGE | | IK |
| | | | VYTLPPSRDELTKNQVSL | | | | C | | |
| | | | WCLVKGFYPSDIAVEWES | | | | | | |
| | | | NGQPENNYKTTPPVLDSD | | | | | | |
| | | | GSFFLYSKLTVDKSRWQQ | | | | | | |
| | | | GNVFSCSVMHEALHNHYT | | | | | | |
| | | | QKSLSLSPGKGGSHHHHH | | | | | | |
| | | | H | | | | | | |
| EPI249 | CEA- | 458 | QVTLRESGPALVKPTQTLT | 459 | QVTLRESGP | 460 | DIQLTQSPSFLSA | 461 | DIQLTQS |
| | CAM6 | | LTCTFSGFSLSTYGIGVGW | | ALVKPTQTL | | SVGDRVTITCKA | | PSFLSAS |
| | | | IRQPPGKALEWLAHIWWN | | TLTCTFSGF | | SQNVGTAVAW | | VGDRVT |
| | | | DNKYYSTSLKTRLTISKDT | | SLSTYGIGV | | YQQKPGKAPKL | | ITCKASQ |
| | | | SKNQVVLTMTNMDPVDT | | GWIRQPPGK | | LIYSASNRYTGV | | NVGTAV |
| | | | ATYYCARISLPYFDYWGQ | | ALEWLAHIW | | PSRFSGSGSGTE | | AWYQQ |
| | | | GTTLTVSSASTKGPSVFPL | | WNDNKYYS | | FTLTISSLQPEDF | | KPGKAP |
| | | | APCSRSTSESTAALGCLVK | | TSLKTRLTI | | ATYYCQQYSSY | | KLLIYSA |
| | | | DYFPEPVTVSWNSGALTS | | SKDTSKNQV | | PLTFGGGTKVEI | | SNRYTG |
| | | | GVHTFPAVLQSSGLYSLSS | | VLTMTNMD | | KRTVAAPSVFIF | | VPSRFSG |
| | | | VVTVPSSNFGTQTYTCNV | | PVDTATYYC | | PPSDEQLKSGTA | | SGSGTEF |
| | | | DHKPSNTKVDKTVEPKSC | | ARISLPYFD | | SVVCLLNNFYPR | | TLTISSL |
| | | | DKTHTCPPCPAPELLGGPS | | YWGQGTTL | | EAKVQWKVDN | | QPEDFA |
| | | | VFLFPPKPKDTLMISRTPE | | TVSS | | ALQSGNSQESVT | | TYYCQQ |
| | | | VTCVVVDVSHEDPEVKFN | | | | EQDSKDSTYSLS | | YSSYPLT |
| | | | WYVDGVEVHNAKTKPRE | | | | STLTLSKADYEK | | FGGGTK |
| | | | EQYNSTYRVVSVLTVLHQ | | | | HKVYACEVTHQ | | VEIK |
| | | | DWLNGKEYKCKVSNKAL | | | | GLSSPVTKSFNR | | |
| | | | PAPIEKTISKAKGQPREPQ | | | | GEC | | |
| | | | VYTLPPSRDELTKNQVSL | | | | | | |
| | | | WCLVKGFYPSDIAVEWES | | | | | | |
| | | | NGQPENNYKTTPPVLDSD | | | | | | |
| | | | GSFFLYSKLTVDKSRWQQ | | | | | | |
| | | | GNVFSCSVMHEALHNHYT | | | | | | |
| | | | QKSLSLSPGKGGSHHHHH | | | | | | |
| | | | H | | | | | | |
| EPI250 | MUC1 | 462 | EVQLVESGGGLVQPGGSM | 463 | EVQLVESGG | 464 | DIVMTQSPLSNP | 465 | DIVMTQ |
| | | | RLSCVASGFPFSNYWMN | | GLVQPGGS | | VTPGEPASISCRS | | SPLSNPV |
| | | | WVRQAPGKGLEWVGEIRL | | MRLSCVAS | | SKSLLHSNGITY | | TPGEPAS |
| | | | KSNNYTTHYAESVKGRFTI | | GFPFSNYW | | FFWYLQKPGQS | | ISCRSSK |
| | | | SRDDSKNSLYLQMNSLKT | | MNWVRQAP | | PQLLIYQMSNLA | | SLLHSN |
| | | | EDTAVYYCTRHYYFDYW | | GKGLEWVG | | SGVPDRFSGSGS | | GITYFF |
| | | | GQGTLVTVSSASTKGPSVF | | EIRLKSNNY | | GTDFTLRISRVE | | WYLQKP |
| | | | PLAPSSKSTSGGTAALGCL | | TTHYAESVK | | AEDVGVYYCAQ | | GQSPQL |
| | | | VKDYFPEPVTVSWNSGAL | | GRFTISRDD | | NLELPPTFGQGT | | LIYQMS |
| | | | TSGVHTFPAVLQSSGLYSL | | SKNSLYLQ | | KVEIKRTVAAPS | | NLASGV |
| | | | SSVVTVPSSSLGTQTYICN | | MNSLKTEDT | | VFIFPPSDEQLKS | | PDRFSGS |
| | | | VNHKPSNTKVDKKVEPKS | | AVYYCTRH | | GTASVVCLLNN | | GSGTDF |
| | | | CDKTHTCPPCPAPELLGGP | | YYFDYWGQ | | FYPREAKVQWK | | TLRISRV |
| | | | SVFLFPPKPKDTLMISRTP | | GTLVTVSS | | VDNALQSGNSQ | | EAEDVG |
| | | | EVTCVVVDVSHEDPEVKFN | | | | ESVTEQDSKDST | | VYYCAQ |
| | | | WYVDGVEVHNAKTKPRE | | | | YSLSSTLTLSKA | | NLELPPT |
| | | | EQYNSTYRVVSVLTVLHQ | | | | DYEKHKVYACE | | FGQGTK |
| | | | DWLNGKEYKCKVSNKAL | | | | VTHQGLSSPVTK | | VEIK |
| | | | PAPIEKTISKAKGQPREPQ | | | | SFNRGEC | | |
| | | | VYTLPPSRDELTKNQVSL | | | | | | |
| | | | WCLVKGFYPSDIAVEWES | | | | | | |
| | | | NGQPENNYKTTPPVLDSD | | | | | | |
| | | | GSFFLYSKLTVDKSRWQQ | | | | | | |
| | | | GNVFSCSVMHEALHNHYT | | | | | | |
| | | | QKSLSLSPGKGGSHHHHH | | | | | | |
| | | | H | | | | | | |
| EPI251 | CD276 | 466 | EVQLVESGGGLVQPGGSL | 467 | EVQLVESGG | 468 | DIQLTQSPSFLSA | 469 | DIQLTQS |
| | | | RLSCAASGFTFSSFGMHW | | GLVQPGGSL | | SVGDRVTITCKA | | PSFLSAS |
| | | | VRQAPGKGLEWVAYISSD | | RLSCAASGF | | SQNVDTNVAW | | VGDRVT |
| | | | SSAIYYADTVKGRFTISRD | | TFSSFGMH | | YQQKPGKAPKA | | ITCKASQ |
| | | | NAKNSLYLQMNSLRDEDT | | WVRQAPGK | | LIYSASYRYSGV | | NVDTNV |
| | | | AVYYCGRGRENIYYGSRL | | GLEWVAYIS | | PSRFSGSGSGTD | | AWYQQ |
| | | | DYWGQGTTVTVSSASTKG | | SDSSAIYYA | | FTLTISSLQPEDF | | KPGKAP |
| | | | PSVFPLAPSSKSTSGGTAA | | DTVKGRFTI | | ATYYCQQYNNY | | KALIYS |
| | | | LGCLVKDYFPEPVTVSWN | | SRDNAKNSL | | PFTFGQGTKLEI | | ASYRYS |
| | | | SGALTSGVHTFPAVLQSSG | | YLQMNSLR | | KRTVAAPSVFIF | | GVPSRFS |
| | | | LYSLSSVVTVPSSSLGTQT | | DEDTAVYY | | PPSDEQLKSGTA | | GSGSGT |
| | | | YICNVNHKPSNTKVDKRV | | CGRGRENIY | | SVVCLLNNFYPR | | DFTLTIS |
| | | | EPKSCDKTHTCPPCPAPEL | | YGSRLDYW | | EAKVQWKVDN | | SLQPEDF |
| | | | LGGPSVFLFPPKPKDTLMI | | GQGTTVTVS | | ALQSGNSQESVT | | ATYYCQ |
| | | | SRTPEVTCVVVDVSHEDP | | S | | EQDSKDSTYSLS | | QYNNYP |
| | | | EVKFNWYVDGVEVHNAK | | | | STLTLSKADYEK | | FTFGQG |
| | | | TKPREEQYNSTYRVVSVL | | | | HKVYACEVTHQ | | TKLEIK |
| | | | TVLHQDWLNGKEYKCKV | | | | GLSSPVTKSFNR | | |
| | | | SNKALPAPIEKTISKAKGQ | | | | GEC | | |
| | | | PREPQVYTLPPSRDELTKN | | | | | | |
| | | | QVSLWCLVKGFYPSDIAV | | | | | | |
| | | | EWESNGQPENNYKTTPPV | | | | | | |
| | | | LDSDGSFFLYSKLTVDKSR | | | | | | |
| | | | WQQGNVFSCSVMHEALH | | | | | | |
| | | | NHYTQKSLSLSPGKGGSH | | | | | | |
| | | | HHHHH | | | | | | |
| EPI252 | CEA- | 470 | QVQLVQSGAEVKKPGASV | 471 | QVQLVQSG | 472 | DIQMTQSPSSLS | 473 | DIQMTQ |
| | CAM6 | | KVSCKASGYTFTDYAMH | | AEVKKPGAS | | ASVGDRVTITCQ | | SPSSLSA |
| | | | WVRQAPGQRLEWMGLIST | | VKVSCKAS | | ASENIYGALNW | | SVGDRV |
| | | | YSGDTKYNQNFQGRVTM | | GYTFTDYA | | YQRKPGKSPKL | | TITCQAS |
| | | | TVDKSASTAYMELSSLRSE | | MHWVRQAP | | LIYGASNLATG | | ENIYGA |
| | | | DTAVYYCARGDYSGSRY | | GQRLEWMG | | MPSRFSGSGSGT | | LNWYQ |
| | | | WFAYWGQGTLVTVSSAST | | LISTYSGDT | | DYTFTISSLQPE | | RKPGKS |
| | | | KGPSVFPLAPSSKSTSGGT | | KYNQNFQG | | DIATYYCQQVLS | | PKLLIYG |
| | | | AALGCLVKDYFPEPVTVS | | RVTMTVDK | | SPYTFGGGTKLE | | ASNLAT |
| | | | WNSGALTSGVHTFPAVLQ | | SASTAYMEL | | IKRTVAAPSVFIF | | GMPSRF |
| | | | SSGLYSLSSVVTVPSSSLG | | SSLRSEDTA | | PPSDEQLKSGTA | | SGSGSG |
| | | | TQTYICNVNHKPSNTKVD | | VYYCARGD | | SVVCLLNNFYPR | | TDYTFTI |
| | | | KKVEPKSCDKTHTCPPCP | | YSGSRYWF | | EAKVQWKVDN | | SSLQPED |
| | | | APELLGGPSVFLFPPKPKD | | AYWGQGTL | | ALQSGNSQESVT | | IATYYC |
| | | | TLMISRTPEVTCVVVDVSH | | VTVSS | | EQDSKDSTYSLS | | QQVLSS |
| | | | EDPEVKFNWYVDGVEVH | | | | STLTLSKADYEK | | PYTFGG |
| | | | NAKTKPREEQYNSTYRVV | | | | HKVYACEVTHQ | | GTKLEIK |
| | | | SVLTVLHQDWLNGKEYK | | | | GLSSPVTKSFNR | | |
| | | | CKVSNKALPAPIEKTISKA | | | | GEC | | |
| | | | KGQPREPQVYTLPPSRDEL | | | | | | |
| | | | TKNQVSLWCLVKGFYPSD | | | | | | |
| | | | LAVEWESNGQPENNYKTT | | | | | | |
| | | | PPVLDSDGSFFLYSKLTVD | | | | | | |
| | | | KSRWQQGNVFSCSVMHE | | | | | | |
| | | | ALHNHYTQKSLSLSPGKG | | | | | | |
| | | | GSHHHHHH | | | | | | |
| EPI253 | ALCAM | 474 | QITLKESGPTLVKPTQTLT | 475 | QITLKESGP | 476 | DIVMTQSPLSLP | 477 | DIVMTQ |
| | | | LTCTFSGFSLSTYGMGVG | | LTVKPTQTL | | VTPGEPASISCRS | | SPLSLPV |
| | | | WIRQPPGKALEWLANIW | | TLTCTFSGF | | SKSLLHSNGITY | | TPGEPAS |
| | | | WSEDKHYSPSLKSRLTITK | | SSLTYGMGVG | | LYWYLQKPGQS | | ISCRSSK |
| | | | DTSKNQVVLTITNVDPVD | | WIRQPPGKA | | PQLLIYQMSNLA | | SLLHSN |
| | | | TATYYCVQIDYGNDYAFT | | LEWLANIW | | SGVPDRFSGSGS | | GITYLY |
| | | | YWGQGTLVTVSSASTKGP | | WSEDKHYS | | GTDFTLKISRVE | | WYLQKP |
| | | | SVFPLAPSSKSTSGGTAAL | | PSLKSRLTI | | AEDVGVYYCAQ | | GQSPQL |
| | | | GCLVKDYFPEPVTVSWNS | | TKDTSKNQV | | NLELPYTFGQGT | | LIYQMS |
| | | | GALTSGVHTFPAVLQSSGL | | VLTITNVDP | | KLEIKRTVAAPS | | NLASGV |
| | | | YSLSSVVTVPSSSLGTQTY | | VDTATYYC | | VFIFPPSDEQLKS | | PDRFSGS |
| | | | ICNVNHKPSNTKVDKKVEP | | VQIDYGND | | GTASVVCLLNN | | GSGTDF |
| | | | KSCDKTHTCPPCPAPELLG | | YAFTYWGQ | | TYPREAKVQWK | | TLKISRV |
| | | | GPSVFLFPPKPKDTLMISR | | GTLVTVSS | | VDNALQSGNSQ | | EAEDVG |
| | | | TPEVTCVVVDVSHEDPEV | | | | ESVTEQDSKDST | | VYYCAQ |
| | | | KFNWYVDGVEVHNAKTK | | | | YSLSSTLTLSKA | | NLELPY |
| | | | PREEQYNSTYRVVSVLTV | | | | DYEKHKVYACE | | TFGQGT |
| | | | LHQDWLNGKEYKCKVSN | | | | VTHQGLSSPVTK | | KLEIK |
| | | | KALPAPIEKTISKAKGQPR | | | | SFNRGEC | | |
| | | | EPQVYTLPPSRDELTKNQV | | | | | | |
| | | | SLWCLVKGFYPSDIAVEW | | | | | | |
| | | | ESNGQPENNYKTTPPVLDS | | | | | | |
| | | | DGSFFLYSKLTVDKSRWQ | | | | | | |
| | | | QGNVFSCSVMHEALHNH | | | | | | |
| | | | YTQKSLSLSPGKGGSHHH | | | | | | |
| | | | HHH | | | | | | |
| EPI254 | PRLR | 478 | EVQLVQSGAEVKKPGSSV | 479 | EVQLVQSG | 480 | DIQMTQSPSSVS | 481 | DIQMTQ |
| | | | KVSCKASGYTFTTYWMH | | AEVKKPGSS | | ASVGDRVTITCK | | SPSSVSA |
| | | | WVRQAPGQGLEWIGEIDP | | VKVSCKAS | | ASQYVGTAVA | | SVGDRV |
| | | | SDSYSNYNQKFKDRATLT | | GYTFTTYW | | WYQQKPGKSPK | | TITCKAS |
| | | | VDKSTSTAYMELSSLRSED | | MHWVRQAP | | LLIYSASNRYTG | | QYVGTA |
| | | | TAVYYCARNGGLGPAWF | | GQGLEWIGE | | VPSRFSDSGSGT | | VAWYQ |
| | | | SYWGQGTLVTVSSASTKG | | IDPSDSYSN | | DFTLTISSLQPED | | QKPGKS |
| | | | PSVFPLAPSSKSTSGGTAA | | YNQKFKDR | | FATYFCQQYSSY | | PKLLIYS |
| | | | LGCLVKDYFPEPVTVSWN | | ATLTVDKST | | PWTFGGGTKVEI | | ASNRYT |
| | | | SGALTSGVHTFPAVLQSSG | | STAYMELSS | | KRTVAAPSVFIF | | GVPSRFS |
| | | | LYSLSSVVTVPSSSLGTQT | | LRSEDTAVY | | PPSDEQLKSGTA | | DSGSGT |
| | | | YICNVNHKPSNTKVDKKV | | YCARNGGL | | SVVCLLNNFYPR | | DFTLTIS |
| | | | EPKSCDKTHTCPPCPAPEL | | GPAWFSYW | | EAKVQWKVDN | | SLQPEDF |
| | | | LGGPSVFLFPPKPKDTLMI | | GQGTLVTVS | | ALQSGNSQESVT | | ATYFCQ |
| | | | SRTPEVTCVVVDVSHEDP | | S | | EQDSKDSTYSLS | | QYSSYP |
| | | | EVKFNWYVDGVEVHNAK | | | | STLTLSKADYEK | | WTFGGG |
| | | | TKPREEQYNSTYRVVSVL | | | | HKVYACEVTHQ | | TKVEIK |
| | | | TVLHQDWLNGKEYKCKV | | | | GLSSPVTKSFNR | | |
| | | | SNKALPAPIEKTISKAKGQ | | | | GEC | | |
| | | | PREPQVYTLPPSRDELTKN | | | | | | |
| | | | QVSLWCLVKGFYPSDIAV | | | | | | |
| | | | EWESNGQPENNYKTTPPV | | | | | | |
| | | | LDSDGSFFLYSKLTVDKSR | | | | | | |
| | | | WQQGNVFSCSVMHEALH | | | | | | |
| | | | NHYTQKSLSLSPGKGGSH | | | | | | |
| | | | HHHHH | | | | | | |
| EPI255 | SLC- | 482 | QVQLVQSGAEVVKPGASV | 483 | QVQLVQSG | 484 | DIQMTQSPSSLS | 485 | DIQMTQ |
| | 34A2 | | KMSCKASGYTFTGYNIHW | | AEVVKPGAS | | ASVGDRVTITCS | | SPSSLSA |
| | | | VKQAPGQGLEWIGAIYPG | | VKMSCKAS | | ASQDIGNFLNW | | SVGDRV |
| | | | NGDTSYKQKFRGRATLTA | | GYTFTGYNI | | YQQKPGKTVKV | | TITCSAS |
| | | | DTSTSTVYMELSSLRSEDS | | HWVKQAPG | | LIYYTSSLYSGV | | QDIGNF |
| | | | AVYYCARGETARATFAY | | QGLEWIGAI | | PSRFSGSGSGTD | | LNWYQ |
| | | | WGQGTLVTVSSGASTKGP | | YPGNGDTSY | | YTLTISSLQPEDF | | QKPGKT |
| | | | SVFPLAPSSKSTSGGTAAL | | KQKFRGRA | | ATYYCQQYSKL | | VKVLIY |
| | | | GCLVKDYFPEPVTVSWNS | | TLTADTSTS | | PLTFGQGTKLEL | | YTSSLYS |
| | | | GALTSGVHTFPAVLQSSGL | | TVYMELSSL | | KRRTVAAPSVFI | | GVPSRFS |
| | | | YSLSSVVTVPSSSLGTQTY | | RSEDSAVYY | | FPPSDEQLKSGT | | GSGSGT |
| | | | ICNVNHKPSNTKVDKRVEP | | CARGETAR | | ASVVCLLNNFY | | DYTLTIS |
| | | | KSCDKTHTCPPCPAPELLG | | ATFAYWGQ | | PREAKVQWKVD | | SLQPEDF |
| | | | GPSVFLFPPKPKDTLMISR | | GTLVTVSS | | NALQSGNSQES | | ATYYCQ |
| | | | TPEVTCVVVDVSHEDPEV | | | | VTEQDSKDSTYS | | QYSKLP |
| | | | KFNWYVDGVEVHNAKTK | | | | LSSTLTLSKADY | | LTFGQG |
| | | | PREEQYNSTYRVVSVLTV | | | | EKHKVYACEVT | | TKLELK |
| | | | LHQDWLNGKEYKCKVSN | | | | HQGLSSPVTKSF | | R |
| | | | KALPAPIEKTISKAKGQPR | | | | NRGEC | | |
| | | | EPQVYTLPPSRDELTKNQV | | | | | | |
| | | | SLWCLVKGFYPSDIAVEW | | | | | | |
| | | | ESNGQPENNYKTTPPVLDS | | | | | | |
| | | | DGSFFLYSKLTVDKSRWQ | | | | | | |
| | | | QGNVFSCSVMHEALHNH | | | | | | |
| | | | YTQKSLSLSPGKGGSHHH | | | | | | |
| | | | HHH | | | | | | |
| EPI256 | ITGB6 | 486 | QVQLVQSGAEVKKPGASV | 487 | QVQLVQSG | 488 | DVVMTQSPLSLP | 489 | DVVMT |
| | | | KVSCKASGYTFTGYFMN | | AEVKKPGAS | | VTLGQPASISCK | | QSPLSLP |
| | | | WVRQAPGQGLEWMGLIN | | VKVSCKAS | | SSQSLLDSDGKT | | VTLGQP |
| | | | PYNGDSFYNQKFKGRVTM | | GYTFTGYF | | YLNWFQQRPGQ | | ASISCKS |
| | | | TRDTSTSTVYMELSSLRSE | | MNWVRQAP | | SPRRLIYLVSEL | | SQSLLDS |
| | | | DTAVYYCARGLRRDFDY | | GQGLEWMG | | DSGVPDRFSGSG | | DGKTYL |
| | | | WGQGTLVTVSSASTKGPS | | LINPYNGDS | | SGTDFTLKISRV | | NWFQQR |
| | | | VFPLAPSSKSTSGGTAALG | | FYNQKFKG | | EAEDVGVYYC | | PGQSPR |
| | | | CLVKDYFPEPVTVSWNSG | | RVTMTRDTS | | WQGTHFPRTFG | | RLIYLVS |
| | | | ALTSGVHTFPAVLQSSGLY | | TSTVYMELS | | GGTKLEIKRTVA | | ELDSGV |
| | | | SLSSVVTVPSSSLGTQTYI | | SLRSEDTAV | | APSVFIFPPSDEQ | | PDRFSGS |
| | | | CNVNHKPSNTKVDKKVEPK | | YYCARGLR | | LKSGTASVVCLL | | GSGTDF |
| | | | SCDKTHTCPPCPAPELLGG | | RDFDYWGQ | | NNFYPREAKVQ | | TLKISRV |
| | | | PSVFLFPPKPKDTLMISRT | | GTLVTVSS | | WKVDNALQSG | | EAEDVG |
| | | | PEVTCVVVDVSHEDPEVKF | | | | NSQESVTEQDSK | | VYYCW |
| | | | NWYVDGVEVHNAKTKPR | | | | DSTYSLSSTLTL | | QGTHFP |
| | | | EEQYNSTYRVVSVLTVLH | | | | SKADYEKHKVY | | RTFGGG |
| | | | QDWLNGKEYKCKVSNKA | | | | ACEVTHQGLSSP | | TKLEIK |
| | | | LPAPIEKTISKAKGQPREP | | | | VTKSFNRGEC | | |
| | | | QVYTLPPSRDELTKNQVSL | | | | | | |
| | | | WCLVKGFYPSDIAVEWES | | | | | | |
| | | | NGQPENNYKTTPPVLDSD | | | | | | |
| | | | GSFFLYSKLTVDKSRWQQ | | | | | | |
| | | | GNVFSCSVMHEALHNHYT | | | | | | |
| | | | QKSLSLSPGKGGSHHHHH | | | | | | |
| | | | H | | | | | | |
| EPI257 | ITGB6 | 490 | QVQLVQSGAEVKKPGASV | 491 | QVQLVQSG | 492 | DVVMTQSPLSLP | 493 | DVVMT |
| | | | KVSCKASGYSFSGYFMNW | | AEVKKPGAS | | VTLGQPASISCK | | QSPLSLP |
| | | | VRQAPGQGLEWMGLINPY | | VKVSCKAS | | SSQSLLDSDGKT | | VTLGQP |
| | | | NGDSFYNQKFKGRVTMTR | | GYSFSGYFM | | YLNWLFQRPGQ | | ASISCKS |
| | | | QTSTSTVYMELSSLRSEDT | | NWVRQAPG | | SPRRLIYLVSEL | | SQSLLDS |
| | | | AVYYCVRGLRRDFDYWG | | QGLEWMGL | | DSGVPDRFSGSG | | DGKTYL |
| | | | QGTLVTVSSASTKGPSVFP | | INPYNGDSF | | SGTDFTLKISRV | | NWLFQR |
| | | | LAPSSKSTSGGTAALGCLV | | YNQKFKGR | | EAEDVGVYYC | | PGQSPR |
| | | | KDYFPEPVTVSWNSGALT | | VTMTRQTST | | WQGTHFPRTFG | | RLIYLVS |
| | | | SGVHTFPAVLQSSGLYSLS | | STVYMELSS | | GGTKLEIKRTVA | | ELDSGV |
| | | | SVVTVPSSSLGTQTYICNV | | LRSEDTAVY | | APSVFIFPPSDEQ | | PDRFSGS |
| | | | NHKPSNTKVDKKVEPKSC | | YCVRGLRR | | LKSGTASVVCLL | | GSGTDF |
| | | | DKTHTCPPCPAPELLGGPS | | DFDYWGQG | | NNFYPREAKVQ | | TLKISRV |
| | | | VFLFPPKPKDTLMISRTPE | | TLVTVSS | | WKVDNALQSG | | EAEDVG |
| | | | VTCVVVDVSHEDPEVKFN | | | | NSQESVTEQDSK | | VYYCW |
| | | | WYVDGVEVHNAKTKPRE | | | | DSTYSLSSTLTL | | QGTHFP |
| | | | EQYNSTYRVVSVLTVLHQ | | | | SKADYEKHKVY | | RTFGGG |
| | | | DWLNGKEYKCKVSNKAL | | | | ACEVTHQGLSSP | | TKLEIK |
| | | | PAPIEKTISKAKGQPREPQ | | | | VTKSFNRGEC | | |
| | | | VYTLPPSRDELTKNQVSL | | | | | | |
| | | | WCLVKGFYPSDIAVEWES | | | | | | |
| | | | NGQPENNYKTTPPVLDSD | | | | | | |
| | | | GSFFLYSKLTVDKSRWQQ | | | | | | |
| | | | GNVFSCSVMHEALHNHYT | | | | | | |
| | | | QKSLSLSPGKGGSHHHHH | | | | | | |
| | | | H | | | | | | |
| EPI258 | ITGB6 | 494 | QVQLVQSGAEVKKPGASV | 495 | QVQLVQSG | 496 | DVVMTQSPLSLP | 497 | DVVMT |
| | | | KVSCKASGYSFSGYFMNW | | AEVKKPGAS | | VTLGQPASISCK | | QSPLSLP |
| | | | VRQAPGQGLEWMGLINPY | | VKVSCKAS | | SSQSLLDSDGKT | | VTLGQP |
| | | | NGDSFYNQKFKGRVTMTR | | GYSFSGYFM | | YLNWLFQRPGQ | | ASISCKS |
| | | | DKSSSTAYMELSSLRSEDT | | NWVRQAPG | | SPRRLIYLVSEL | | SQSLLDS |
| | | | AVYYCARGLRRDFDYWG | | QGLEWMGL | | DSGVPDRFSGSG | | DGKTYL |
| | | | QGTLVTVSSASTKGPSVFP | | INPYNGDSF | | SGTDFTLKISRV | | NWLFQR |
| | | | LAPSSKSTSGGTAALGCLV | | YNQKFKGR | | EAEDVGVYYC | | PGQSPR |
| | | | KDYFPEPVTVSWNSGALT | | VTMTRDKSS | | WQGTHFPRTFG | | RLIYLVS |
| | | | SGVHTFPAVLQSSGLYSLS | | STAYMELSS | | GGTKLEIKRTVA | | ELDSGV |
| | | | SVVTVPSSSLGTQTYICNV | | LRSEDTAVY | | APSVFIFPPSDEQ | | PDRFSGS |
| | | | NHKPSNTKVDKKVEPKSC | | YCARGLRR | | LKSGTASVVCLL | | GSGTDF |
| | | | DKTHTCPPCPAPELLGGPS | | DFDYWGQG | | NNFYPREAKVQ | | TLKISRV |
| | | | VFLFPPKPKDTLMISRTPE | | TLVTVSS | | WKVDNALQSG | | EAEDVG |
| | | | VTCVVVDVSHEDPEVKFN | | | | NSQESVTEQDSK | | VYYCW |
| | | | WYVDGVEVHNAKTKPRE | | | | DSTYSLSSTLTL | | QGTHFP |
| | | | EQYNSTYRVVSVLTVLHQ | | | | SKADYEKHKVY | | RTFGGG |
| | | | DWLNGKEYKCKVSNKAL | | | | ACEVTHQGLSSP | | TKLEIK |
| | | | PAPIEKTISKAKGQPREPQ | | | | VTKSFNRGEC | | |
| | | | VYTLPPSRDELTKNQVSL | | | | | | |
| | | | WCLVKGFYPSDIAVEWES | | | | | | |
| | | | NGQPENNYKTTPPVLDSD | | | | | | |
| | | | GSFFLYSKLTVDKSRWQQ | | | | | | |
| | | | GNVFSCSVMHEALHNHYT | | | | | | |
| | | | QKSLSLSPGKGGSHHHHH | | | | | | |
| | | | H | | | | | | |
| EPI259 | CD71 | 498 | QVQLVQSGAEVKKPGASV | 499 | QVQLVQSG | 500 | DIQMTQSPSSLS | 501 | DIQMTQ |
| | | | KMSCKASGYTFTSYWMH | | AEVKKPGAS | | ASVGDRVTITCS | | SPSSLSA |
| | | | WVRQAPGQGLEWIGAIYP | | VKMSCKAS | | ASSSVYYMYWF | | SVGDRV |
| | | | GNSETGYAQKFQGRATLT | | GYTFTSYW | | QQKPGKAPKLW | | TITCSAS |
| | | | ADTSTSTAYMELSSLRSED | | MHWVRQAP | | IYSTSNLASGVP | | SSVYYM |
| | | | TAVYYCTRENWDPGFAF | | GQGLEWIG | | SRFSGSGSGTDY | | YWFQQ |
| | | | WGQGTLITVSSASTKGPSV | | AIYPGNSET | | TLTISSMQPEDF | | KPGKAP |
| | | | FPLAPSSKSTSGGTAALGC | | GYAQKFQG | | ATYYCQQRRNY | | KLWIYS |
| | | | LVKDYFPEPVTVSWNSGA | | RATLTADTS | | PYTFGQGTKLEI | | TSNLAS |
| | | | LTSGVHTFPAVLQSSGLYS | | TSTAYMELS | | KRTVAAPSVFIF | | GVPSRFS |
| | | | LSSVVTVPSSSLGTQTYIC | | SLRSEDTAV | | PPSDEQLKSGTA | | GSGSGT |
| | | | NVNHKPSNTKVDKKVEPK | | YYCTRENW | | SVVCLLNNFYPR | | DYTLTIS |
| | | | SCDKTHTCPPCPAPELLGG | | DPGFAFWG | | EAKVQWKVDN | | SMQPED |
| | | | PSVFLFPPKPKDTLMISRT | | QGTLITVSS | | ALQSGNSQESVT | | FATYYC |
| | | | PEVTCVVVDVSHEDPEVKF | | | | EQDSKDSTYSLS | | QQRRNY |
| | | | NWYVDGVEVHNAKTKPR | | | | STLTLSKADYEK | | PYTFGQ |
| | | | EEQYNSTYRVVSVLTVLH | | | | HKVYACEVTHQ | | GTKLEIK |
| | | | QDWLNGKEYKCKVSNKA | | | | GLSSPVTKSFNR | | |
| | | | LPAPIEKTISKAKGQPREP | | | | GEC | | |
| | | | QVYTLPPSRDELTKNQVSL | | | | | | |
| | | | WCLVKGFYPSDIAVEWES | | | | | | |
| | | | NGQPENNYKTTPPVLDSD | | | | | | |
| | | | GSFFLYSKLTVDKSRWQQ | | | | | | |
| | | | GNVFSCSVMHEALHNHYT | | | | | | |
| | | | QKSLSLSPGKGGSHHHHH | | | | | | |
| | | | H | | | | | | |
| EPI260 | HER3 | 502 | EVQLLESGGGLVQPGGSL | 503 | EVQLLESGG | 504 | QSALTQPASVSG | 505 | QSALTQ |
| | | | RLSCAASGFTFSHYVMAW | | GLVQPGGSL | | SPGQSITISCTGT | | PASVSG |
| | | | VRQAPGKGLEWVSSISSSG | | RLSCAASGF | | SSDVGSYNVVS | | SPGQSIT |
| | | | GWTLYADSVKGRFTISRD | | TFSHYVMA | | WYQQHPGKAPK | | ISCTGTS |
| | | | NSKNTLYLQMNSLRAEDT | | WVRQAPGK | | LIIYEVSQRPSGV | | SDVGSY |
| | | | AVYYCTRGLKMATIFDY | | GLEWVSSIS | | SNRFSGSKSGNT | | NVVSW |
| | | | WGQGTLVTVSSASTKGPS | | SSGGWTLY | | ASLTISGLQTED | | YQQHPG |
| | | | VFPLAPCSRSTSESTAALG | | ADSVKGRFT | | EADYYCCSYAG | | KAPKLII |
| | | | CLVKDYFPEPVTVSWNSG | | ISRDNSKNT | | SSIFVIFGGGTKV | | YEVSQR |
| | | | ALTSGVHTFPAVLQSSGLY | | LYLQMNSL | | TVLGQPKAAPS | | PSGVSN |
| | | | SLSSVVTVPSSNFGTQTYT | | RAEDTAVY | | VTLFPPSSEELQ | | RFSGSKS |
| | | | CNVDHKPSNTKVDKTVEP | | YCTRGLKM | | ANKATLVCLVS | | GNTASL |
| | | | KSCDKTHTCPPCPAPELLG | | ATIFDYWGQ | | DFYPGAVTVAW | | TISGLQT |
| | | | GPSVFLFPPKPKDTLMISR | | GTLVTVSS | | KADGSPVKVGV | | EDEADY |
| | | | TPEVTCVVVDVSHEDPEV | | | | ETTKPSKQSNNK | | YCCSYA |
| | | | KFNWYVDGVEVHNAKTK | | | | YAASSYLSLTPE | | GSSIFVIF |
| | | | PREEQYNSTYRVVSVLTV | | | | QWKSHRSYSCR | | GGGTKV |
| | | | LHQDWLNGKEYKCKVSN | | | | VTHEGSTVEKT | | TVL |
| | | | KALPAPIEKTISKAKGQPR | | | | VAPAECS | | |
| | | | EPQVYTLPPSRDELTKNQV | | | | | | |
| | | | SLWCLVKGFYPSDIAVEW | | | | | | |
| | | | ESNGQPENNYKTTPPVLDS | | | | | | |
| | | | DGSFFLYSKLTVDKSRWQ | | | | | | |
| | | | QGNVFSCSVMHEALHNH | | | | | | |
| | | | YTQKSLSLSPGKGGSHHH | | | | | | |
| | | | HHH | | | | | | |
| EPI261 | MUC16 | 506 | EVQLVESGGGLVQPGGSL | 507 | EVQLVESGG | 508 | DIQMTQSPSSLS | 509 | DIQMTQ |
| | | | RLSCAASGYSITNDYAWN | | GLVQPGGSL | | ASVGDRVTITCK | | SPSSLSA |
| | | | WVRQAPGKGLEWVGYIS | | RLSCAASGY | | ASDLIHNWLAW | | SVGDRV |
| | | | YSGYTTYNPSLKSRFTISR | | SITNDYAWN | | YQQKPGKAPKL | | TITCKAS |
| | | | DTSKNTLYLQMNSLRAED | | WVRQAPGK | | LIYGATSLETGV | | DLIHNW |
| | | | TAVYYCARWTSGLDYWG | | GLEWVGYIS | | PSRFSGSGSGTD | | LAWYQ |
| | | | QGTLVTVSSASTKGPSVFP | | YSGYTTYNP | | FTLTISSLQPEDF | | QKPGKA |
| | | | LAPSSKSTSGGTAALGCLV | | SLKSRFTISR | | ATYYCQQYWTT | | PKLLIYG |
| | | | KDYFPEPVTVSWNSGALT | | DTSKNTLYL | | PFTFGQGTKVEI | | ATSLET |
| | | | SGVHTFPAVLQSSGLYSLS | | QMNSLRAE | | KRTVAAPSVFIF | | GVPSRFS |
| | | | SVVTVPSSSLGTQTYICNV | | DTAVYYCA | | PPSDEQLKSGTA | | GSGSGT |
| | | | NHKPSNTKVDKKVEPKSC | | RWTSGLDY | | SVVCLLNNFYPR | | DFTLTIS |
| | | | DKTHTCPPCPAPELLGGPS | | WGQGTLVT | | EAKVQWKVDN | | SLQPEDF |
| | | | VFLFPPKPKDTLMISRTPE | | VSS | | ALQSGNSQESVT | | ATYYCQ |
| | | | VTCVVVDVSHEDPEVKFN | | | | EQDSKDSTYSLS | | QYWTTP |
| | | | WYVDGVEVHNAKTKPRE | | | | STLTLSKADYEK | | FTFGQG |
| | | | EQYNSTYRVVSVLTVLHQ | | | | HKVYACEVTHQ | | TKVEIK |
| | | | DWLNGKEYKCKVSNKAL | | | | GLSSPVTKSFNR | | |
| | | | PAPIEKTISKAKGQPREPQ | | | | GEC | | |
| | | | VYTLPPSRDELTKNQVSL | | | | | | |
| | | | WCLVKGFYPSDIAVEWES | | | | | | |
| | | | NGQPENNYKTTPPVLDSD | | | | | | |
| | | | GSFFLYSKLTVDKSRWQQ | | | | | | |
| | | | GNVFSCSVMHEALHNHYT | | | | | | |
| | | | QKSLSLSPGKGGSHHHHH | | | | | | |
| | | | H | | | | | | |
| EPI263 | RSV F | 510 | QVTLRESGPALVKPTQTLT | 511 | QVTLRESGP | 512 | DIQMTQSPSTLS | 513 | DIQMTQ |
| | Pro- | | LTCTFSGFSLSTAGMSVG | | ALVKPTQTL | | ASVGDRVTITCS | | SPSTLSA |
| | tein | | WIRQPPGKALEWLADIW | | TLTCTFSGF | | ASSRVGYMHW | | SVGDRV |
| | | | WDDKKHYNPSLKDRLTIS | | SLSTAGMSV | | YQQKPGKAPKL | | TITCSAS |
| | | | KDTSKNQVVLKVTNMDP | | GWIRQPPGK | | LIYDTSKLASGV | | SRVGYM |
| | | | ADTATYYCARDMIFNFYF | | ALEWLADI | | PSRFSGSGSGTE | | HWYQQ |
| | | | DVWGQGTTVTVSSAKTTA | | WWDDKKH | | FTLTISSLQPDDF | | KPGKAP |
| | | | PSVYPLAPVCGDTTGSSVT | | YNPSLKDRL | | ATYYCFQGSGY | | KLLIYDT |
| | | | LGCLVKGYFPEPVTLTWN | | TISKDTSKN | | PFTFGGGTKVEI | | SKLASG |
| | | | SGSLSSGVHTFPAVLQSDL | | QVVLKVTN | | KRADAAPTVSIF | | VPSRFSG |
| | | | YTLSSSVTVTSSTWPSQSI | | MDPADTAT | | PPSSEQLTSGGA | | SGSGTEF |
| | | | TCNVAHPASSTKVDKKIEP | | YYCARDMIF | | SVVCFLNNFYPK | | TLTISSL |
| | | | KSCDKTHTCPPCPAPELLG | | NFYFDVWG | | DINVKWKIDGSE | | QPDDFA |
| | | | GPSVFLFPPKPKDTLMISR | | QGTTVTVSS | | RQNGVLNSWTD | | TYYCFQ |
| | | | TPEVTCVVVDVSHEDPEV | | | | QDSKDSTYSMS | | GSGYPF |
| | | | KFNWYVDGVEVHNAKTK | | | | STLTLTKDEYER | | TFGGGT |
| | | | PREEQYNSTYRVVSVLTV | | | | HNSYTCEATHK | | KVEIK |
| | | | LHQDWLNGKEYKCKVSN | | | | TSTSPIVKSFNR | | |
| | | | KALPAPIEKTISKAKGQPR | | | | NEC | | |
| | | | EPQVYTLPPSRDELTKNQV | | | | | | |
| | | | SLWCLVKGFYPSDIAVEW | | | | | | |
| | | | ESNGQPENNYKTTPPVLDS | | | | | | |
| | | | DGSFFLYSKLTVDKSRWQ | | | | | | |
| | | | QGNVFSCSVMHEALHNH | | | | | | |
| | | | YTQKSLSLSPGKGGSHHH | | | | | | |
| | | | HHH | | | | | | |
| EPI338 | LRRC15 | 514 | EVQLVQSGAEVKKPGASV | 515 | EVQLVQSG | 516 | DIQMTQSPSSLS | 517 | DIQMTQ |
| | | | KVSCKASGYKFSSYWIEW | | AEVKKPGAS | | ASVGDRVTITCR | | SPSSLSA |
| | | | VKQAPGQGLEWIGEILPGS | | VKVSCKAS | | ASQDISNYLNW | | SVGDRV |
| | | | DTTNYNEKFKDRATFTSD | | GYKFSSYWI | | YQQKPGGAVKF | | TITCRAS |
| | | | TSINTAYMELSRLRSDDTA | | EWVKQAPG | | LIYYTSRLHSGV | | QDISNY |
| | | | VYYCARDRGNYRAWFGY | | QGLEWIGEI | | PSRFSGSGSGTD | | LNWYQ |
| | | | WGQGTLVTVSSASTKGPS | | LPGSDTTNY | | YTLTISSLQPEDF | | QKPGGA |
| | | | VLPLAPSSKSTSGGTAALG | | NEKFKDRAT | | ATYFCQQGEAL | | VKFLIY |
| | | | CLVKDYFPEPVTVSWNSG | | FTSDTSINT | | PWTFGGGTKVEI | | YTSRLH |
| | | | ALTSGVHTFPAVLQSSGLY | | AYMELSRLR | | KRTVAAPSVFIF | | SGVPSRF |
| | | | SLSSVVTVPSSSLGTQTYI | | DSDTAVYYC | | PPSDEQLKSGTA | | SGSGSG |
| | | | CNVNHKPSNTKVDKKVEPK | | ARDRGNYR | | SVVCLLNNFYPR | | TDYTLTI |
| | | | SCDKTHTCPPCPAPELLGG | | AWFGYWGQ | | EAKVQWKVDN | | SSLQPED |
| | | | PSVFLFPPKPKDTLMISRT | | GTLVTVSS | | ALQSGNSQESVT | | FATYFC |
| | | | PEVTCVVVDVSHEDPEVKF | | | | EQDSKDSTYSLS | | QQGEAL |
| | | | NWYVDGVEVHNAKTKPR | | | | STLTLSKADYEK | | PWTFGG |
| | | | EEQYNSTYRVVSVLTVLH | | | | HKVYACEVTHQ | | GTKVEI |
| | | | QDWLNGKEYKCKVSNKA | | | | GLSSPVTKSFNR | | K |
| | | | LPAPIEKTISKAKGQPREP | | | | GEC | | |
| | | | QVYTLPPSRDELTKNQVSL | | | | | | |
| | | | WCLVKGFYPSDIAVEWES | | | | | | |
| | | | NGQPENNYKTTPPVLDSD | | | | | | |
| | | | GSFFLYSKLTVDKSRWQQ | | | | | | |
| | | | GNVFSCSVMHEALHNHYT | | | | | | |
| | | | QKSLSLSPGKGGSHHHHH | | | | | | |
| | | | H | | | | | | |
| EPI358 | RNF43 | 518 | EVQLVESGGGLVQPGGSL | 519 | EVQLVESGG | 520 | DIQMTQSPSSLS | 521 | DIQMTQ |
| | | | RLSCVVSGFTFSYYDMHW | | GLVQPGGSL | | ASVGDRVTITCR | | SPSSLSA |
| | | | VRQVTGKGLEWVSAIGTA | | RLSCVVSGF | | ASQSISSYLNWY | | SVGDRV |
| | | | GATYYPGSVKGRFTISREN | | TFSYYDMH | | QQKPGKAPKLLI | | TITCRAS |
| | | | AKNSLYLQMNSLRAGDTA | | WVRQVTGK | | YAASSLQSGVPS | | QSISSYL |
| | | | VYYCARDRGYSGYDAYY | | GLEWVSAIG | | RFSGSGSGTDFT | | NWYQQ |
| | | | FDFWGQGTLVTVSSASTK | | TAGATYYP | | LTISSLQPEDFAT | | KPGKAP |
| | | | GPSVFPLAPSSKSTSGGTA | | GSVKGRFTI | | YYCQQSYSTPPT | | KLLIYA |
| | | | ALGCLVKDYFPEPVTVSW | | SRENAKNSL | | FGQGTKVEIKRT | | ASSLQS |
| | | | NSGALTSGVHTFPAVLQSS | | YLQMNSLR | | VAAPSVEIFPPS | | GVPSRFS |
| | | | GLYSLSSVVTVPSSSLGTQ | | AGDTAVYY | | DEQLKSGTASV | | GSGSGT |
| | | | TYICNVNHKPSNTKVDKK | | CARDRGYS | | VCLLNNFYPRE | | DFTLTIS |
| | | | VEPKSCDKTHTCPPCPAPE | | GYDAYYFD | | AKVQWKVDNA | | SLQPEDF |
| | | | LLGGPSVFLFPPKPKDTLM | | FWGQGTLV | | LQSGNSQESVTE | | ATYYCQ |
| | | | ISRTPEVTCVVVDVSHEDP | | TVSS | | QDSKDSTYSLSS | | QSYSTPP |
| | | | EVKFNWYVDGVEVHNAK | | | | TLTLSKADYEK | | TFGQGT |
| | | | TKPREEQYNSTYRVVSVL | | | | HKVYACEVTHQ | | KVEIK |
| | | | TVLHQDWLNGKEYKCKV | | | | GLSSPVTKSFNR | | |
| | | | SNKALPAPIEKTISKAKGQ | | | | GEC | | |
| | | | PREPQVYTLPPSRDELTKN | | | | | | |
| | | | QVSLWCLVKGFYPSDIAV | | | | | | |
| | | | JEWESNGQPENNYKTTPPV | | | | | | |
| | | | LDSDGSFFLYSKLTVDKSR | | | | | | |
| | | | WQQGNVFSCSVMHEALH | | | | | | |
| | | | NHYTQKSLSLSPGKGGSH | | | | | | |
| | | | HHHHH | | | | | | |
| EPI359 | RNF43 | 522 | EVQLVQSGGGLVQPGGSL | 523 | EVQLVQSG | 524 | DIQMTQSPSSLS | 525 | DIQMTQ |
| | | | RLSCAASGFTFSYYDMHW | | GGLVQPGGS | | ASVGDRVTITCR | | SPSSLSA |
| | | | VRQVTGKGLEWVSTIGAT | | LRLSCAASG | | ASQSISSYLNWY | | SVGDRV |
| | | | GDTYYSDSVKGRFTISRQN | | FTFSYYDMH | | QQKPGKAPKLLI | | TITCRAS |
| | | | AKNSLYLQINSLRAGDTA | | WVRQVTGK | | YAASSLQSGVPS | | QSISSYL |
| | | | VYYCVRDRGYIGYDSYYF | | GLEWVSTIG | | RFSGSGSGTDFT | | NWYQQ |
| | | | DNWGQGTLVTVSSASTKG | | ATGDTYYS | | LTISSLQPEDFAT | | KPGKAP |
| | | | PSVFPLAPSSKSTSGGTAA | | DSVKGRFTI | | YYCQQSYSTPPT | | KLLIYA |
| | | | LGCLVKDYFPEPVTVSWN | | SRQNAKNSL | | FGQGTKVEIKRT | | ASSLQS |
| | | | SGALTSGVHTFPAVLQSSG | | YLQINSLRA | | VAAPSVEIFPPS | | GVPSRFS |
| | | | LYSLSSVVTVPSSSLGTQT | | GDTAVYYC | | DEQLKSGTASV | | GSGSGT |
| | | | YICNVNHKPSNTKVDKKV | | VRDRGYIGY | | VCLLNNFYPRE | | DFTLTIS |
| | | | EPKSCDKTHTCPPCPAPEL | | DSYYFDNW | | AKVQWKVDNA | | SLQPEDF |
| | | | LGGPSVFLFPPKPKDTLMI | | GQGTLVTVS | | LQSGNSQESVTE | | ATYYCQ |
| | | | SRTPEVTCVVVDVSHEDP | | S | | QDSKDSTYSLSS | | QSYSTPP |
| | | | EVKFNWYVDGVEVHNAK | | | | TLTLSKADYEK | | TFGQGT |
| | | | TKPREEQYNSTYRVVSVL | | | | HKVYACEVTHQ | | KVEIK |
| | | | TVLHQDWLNGKEYKCKV | | | | GLSSPVTKSFNR | | |
| | | | SNKALPAPIEKTISKAKGQ | | | | GEC | | |
| | | | PREPQVYTLPPSRDELTKN | | | | | | |
| | | | QVSLWCLVKGFYPSDIAV | | | | | | |
| | | | EWESNGQPENNYKTTPPV | | | | | | |
| | | | LDSDGSFFLYSKLTVDKSR | | | | | | |
| | | | WQQGNVFSCSVMHEALH | | | | | | |
| | | | NHYTQKSLSLSPGKGGSH | | | | | | |
| | | | HHHHH | | | | | | |
| EPI430 | CEA- | 526 | EVQLVESGGGVVQPGRSL | 527 | EVQLVESGG | 528 | DIQLTQSPSSLSA | 529 | DIQLTQS |
| | CAM5 | | RLSCSASGFDFTTYWMSW | | GVVQPGRSL | | SVGDRVTITCKA | | PSSLSAS |
| | | | VRQAPGKGLEWIGEIHPDS | | RLSCSASGF | | SQDVGTSVAWY | | VGDRVT |
| | | | STINYAPSLKDRFTISRDN | | DFTTYWMS | | QQKPGKAPKLLI | | ITCKASQ |
| | | | AKNTLFLQMDSLRPEDTG | | WVRQAPGK | | YWTSTRHTGVP | | DVGTSV |
| | | | VYFCASLYFGFPWFAYWG | | GLEWIGEIH | | SRFSGSGSGTDF | | AWYQQ |
| | | | QGTPVTVSSASTKGPSVFP | | PDSSTINYA | | TFTISSLQPEDIA | | KPGKAP |
| | | | LAPSSKSTSGGTAALGCLV | | PSLKDRFTI | | TYYCQQYSLYR | | KLLIYW |
| | | | KDYFPEPVTVSWNSGALT | | SRDNAKNTL | | SFGQGTKVEIKR | | TSTRHT |
| | | | SGVHTFPAVLQSSGLYSLS | | FLQMDSLRP | | TVAAPSVFIFPPS | | GVPSRFS |
| | | | SVVTVPSSSLGTQTYICNV | | EDTGVYFCA | | DEQLKSGTASV | | GSGSGT |
| | | | NHKPSNTKVDKRVEPKSC | | SLYFGFPWF | | VCLLNNFYPRE | | DFTFTIS |
| | | | DKTHTCPPCPAPELLGGPS | | AYWGQGTPV | | AKVQWKVDNA | | SLQPEDI |
| | | | VFLFPPKPKDTLMISRTPE | | TVSS | | LQSGNSQESVTE | | ATYYCQ |
| | | | VTCVVVDVSHEDPEVKFN | | | | QDSKDSTYSLSS | | QYSLYR |
| | | | WYVDGVEVHNAKTKPRE | | | | TLTLSKADYEK | | SFGQGT |
| | | | EQYNSTYRVVSVLTVLHQ | | | | HKVYACEVTHQ | | KVEIK |
| | | | DWLNGKEYKCKVSNKAL | | | | GLSSPVTKSFNR | | |
| | | | PAPIEKTISKAKGQPREPQ | | | | GEC | | |
| | | | VYTLPPSRDELTKNQVSL | | | | | | |
| | | | WCLVKGFYPSDIAVEWES | | | | | | |
| | | | NGQPENNYKTTPPVLDSD | | | | | | |
| | | | GSFFLYSKLTVDKSRWQQ | | | | | | |
| | | | GNVFSCSVMHEALHNHYT | | | | | | |
| | | | QKSLSLSPGK | | | | | | |
| EPI437 | ZNRF3 | 530 | QVQLVQSGSELKKPGASV | 531 | QVQLVQSGS | 532 | DIQMTQSPSSLS | 533 | DIQMTQ |
| | | | KVSCKASGYTFTRYPMN | | ELKKPGASV | | ASVGDRVTITCR | | SPSSLSA |
| | | | WVRQAPGQGLEWMGWIN | | KVSCKASG | | ASQSISSYLNWY | | SVGDRV |
| | | | TNTGNPTYAQGFTGRFVF | | YTFTRYPMN | | QQKPGKAPKLLI | | TITCRAS |
| | | | SLDTSVSTAFLQISSLKAE | | WVRQAPGQ | | YAASSLQSGVPS | | QSISSYL |
| | | | DTAVYYCARERTNFYDAF | | GLEWMGWI | | RFSGSGSGTDFT | | NWYQQ |
| | | | DIWGQGTMVTVSSASTKG | | NTNTGNPTY | | LTISSLQPEDFAT | | KPGKAP |
| | | | PSVFPLAPSSKSTSGGTAA | | AQGFTGRFV | | YYCQQSYSTPPT | | KLLIYA |
| | | | LGCLVKDYFPEPVTVSWN | | FSLDTSVST | | FGQGTKVEIKRT | | ASSLQS |
| | | | SGALTSGVHTFPAVLQSSG | | AFLQISSLK | | VAAPSVEIFPPS | | GVPSRFS |
| | | | LYSLSSVVTVPSSSLGTQT | | AEDTAVYY | | DEQLKSGTASV | | GSGSGT |
| | | | YICNVNHKPSNTKVDKKV | | CARERTNFY | | VCLLNNFYPRE | | DFTLTIS |
| | | | EPKSCDKTHTCPPCPAPEL | | DAFDIWGQ | | AKVQWKVDNA | | SLQPEDF |
| | | | LGGPSVFLFPPKPKDTLMI | | GTMVTVSS | | LQSGNSQESVTE | | ATYYCQ |
| | | | SRTPEVTCVVVDVSHEDP | | | | QDSKDSTYSLSS | | QSYSTPP |
| | | | EVKFNWYVDGVEVHNAK | | | | TLTLSKADYEK | | TFGQGT |
| | | | TKPREEQYNSTYRVVSVL | | | | HKVYACEVTHQ | | KVEIK |
| | | | TVLHQDWLNGKEYKCKV | | | | GLSSPVTKSFNR | | |
| | | | SNKALPAPIEKTISKAKGQ | | | | GEC | | |
| | | | PREPQVYTLPPSRDELTKN | | | | | | |
| | | | QVSLWCLVKGFYPSDIAV | | | | | | |
| | | | EWESNGQPENNYKTTPPV | | | | | | |
| | | | LDSDGSFFLYSKLTVDKSR | | | | | | |
| | | | WQQGNVFSCSVMHEALH | | | | | | |
| | | | NHYTQKSLSLSPGKGGSH | | | | | | |
| | | | HHHHH | | | | | | |
| EPI438 | ZNRF3 | 534 | QVQLVQSGSELKKPGASV | 535 | QVQLVQSGS | 536 | DIQMTQSPSSLS | 537 | DIQMTQ |
| | | | KVSCKASGYTFNSYAMD | | ELKKPGASV | | ASVGDRVTITCR | | SPSSLSA |
| | | | WVRQAPGQGLEWMGWIN | | KVSCKASG | | ASQSISSYLNWY | | SVGDRV |
| | | | TNTGNPTYAQAFTGRFVF | | YTFNSYAM | | QQKPGKAPKLLI | | TITCRAS |
| | | | SLDTSVSTAYLEISSLKAE | | DWVRQAPG | | YAASSLQSGVPS | | QSISSYL |
| | | | DTAVYYCARERHGYFEAF | | QGLEWMG | | RFSGSGSGTDFT | | NWYQQ |
| | | | DIWGQGTTVTVSSASTKG | | WINTNTGNP | | LTISSLQPEDFAT | | KPGKAP |
| | | | PSVFPLAPSSKSTSGGTAA | | TYAQAFTGR | | YYCQQSYSTPPT | | KLLIYA |
| | | | LGCLVKDYFPEPVTVSWN | | FVFSLDTSV | | FGQGTKVEIKRT | | ASSLQS |
| | | | SGALTSGVHTFPAVLQSSG | | STAYLEISSL | | VAAPSVEIFPPS | | GVPSRFS |
| | | | LYSLSSVVTVPSSSLGTQT | | KAEDTAVY | | DEQLKSGTASV | | GSGSGT |
| | | | YICNVNHKPSNTKVDKKV | | YCARERHG | | VCLLNNFYPRE | | DFTLTIS |
| | | | EPKSCDKTHTCPPCPAPEL | | YFEAFDIWG | | AKVQWKVDNA | | SLQPEDF |
| | | | LGGPSVFLFPPKPKDTLMI | | QGTTVTVSS | | LQSGNSQESVTE | | ATYYCQ |
| | | | SRTPEVTCVVVDVSHEDP | | | | QDSKDSTYSLSS | | QSYSTPP |
| | | | EVKFNWYVDGVEVHNAK | | | | TLTLSKADYEK | | TFGQGT |
| | | | TKPREEQYNSTYRVVSVL | | | | HKVYACEVTHQ | | KVEIK |
| | | | TVLHQDWLNGKEYKCKV | | | | GLSSPVTKSFNR | | |
| | | | SNKALPAPIEKTISKAKGQ | | | | GEC | | |
| | | | PREPQVYTLPPSRDELTKN | | | | | | |
| | | | QVSLWCLVKGFYPSDIAV | | | | | | |
| | | | EWESNGQPENNYKTTPPV | | | | | | |
| | | | LDSDGSFFLYSKLTVDKSR | | | | | | |
| | | | WQQGNVFSCSVMHEALH | | | | | | |
| | | | NHYTQKSLSLSPGKGGSH | | | | | | |
| | | | HHHHH | | | | | | |
| EPI439 | ZNRF3 | 538 | EVQLVQSGSELKKPGASV | 539 | EVQLVQSGS | 540 | DIQMTQSPSSLS | 541 | DIQMTQ |
| | | | KVSCKASGYTFTKYAMN | | ELKKPGASV | | ASVGDRVTITCR | | SPSSLSA |
| | | | WVRQVPGQGLEWMGWIN | | KVSCKASG | | ASQSISSYLNWY | | SVGDRV |
| | | | TNTGNPTYAQGFTGRFVF | | YTFTKYAM | | QQKPGKAPKLLI | | TITCRAS |
| | | | SLDTSVRTAYLQISSLKAE | | NWVRQVPG | | YAASSLQSGVPS | | QSISSYL |
| | | | DTAVYYCARKGGSYYDW | | QGLEWMG | | RFSGSGSGTDFT | | NWYQQ |
| | | | FDPWGQGTLVTVSSASTK | | WINTNTGNP | | LTISSLQPEDFAT | | KPGKAP |
| | | | GPSVFPLAPSSKSTSGGTA | | TYAQGFTGR | | YYCQQSYSTPPT | | KLLIYA |
| | | | ALGCLVKDYFPEPVTVSW | | FVFSLDTSV | | FGQGTKVEIKRT | | ASSLQS |
| | | | NSGALTSGVHTFPAVLQSS | | RTAYLQISS | | VAAPSVEIFPPS | | GVPSRFS |
| | | | GLYSLSSVVTVPSSSLGTQ | | LKAEDTAV | | DEQLKSGTASV | | GSGSGT |
| | | | TYICNVNHKPSNTKVDKK | | YYCARKGG | | VCLLNNFYPRE | | DFTLTIS |
| | | | VEPKSCDKTHTCPPCPAPE | | SYYDWFDP | | AKVQWKVDNA | | SLQPEDF |
| | | | LLGGPSVFLFPPKPKDTLM | | WGQGTLVT | | LQSGNSQESVTE | | ATYYCQ |
| | | | ISRTPEVTCVVVDVSHEDP | | VSS | | QDSKDSTYSLSS | | QSYSTPP |
| | | | EVKFNWYVDGVEVHNAK | | | | TLTLSKADYEK | | TFGQGT |
| | | | TKPREEQYNSTYRVVSVL | | | | HKVYACEVTHQ | | KVEIK |
| | | | TVLHQDWLNGKEYKCKV | | | | GLSSPVTKSFNR | | |
| | | | SNKALPAPIEKTISKAKGQ | | | | GEC | | |
| | | | PREPQVYTLPPSRDELTKN | | | | | | |
| | | | QVSLWCLVKGFYPSDIAV | | | | | | |
| | | | EWESNGQPENNYKTTPPV | | | | | | |
| | | | LDSDGSFFLYSKLTVDKSR | | | | | | |
| | | | WQQGNVFSCSVMHEALH | | | | | | |
| | | | NHYTQKSLSLSPGKGGSH | | | | | | |
| | | | HHHHH | | | | | | |
| EPI440 | LGR5 | 542 | EVQLVQSGSKLKKPGASV | 543 | EVQLVQSGS | 544 | DIQMTQSPSSLS | 545 | DIQMTQ |
| | | | KVSCKASGYTFTSYTMNW | | KLKKPGASV | | ASVGDRVTITCR | | SPSSLSA |
| | | | VRQAPGQGLEWMGWINT | | KVSCKASG | | ASQSISSYLNWY | | SVGDRV |
| | | | DTGDPTYAQGFTGRFVFS | | YTFTSYTMN | | QQKPGKAPKLLI | | TITCRAS |
| | | | LDTSVSTAFLQINSLKAED | | WVRQAPGQ | | YAASSLQSGVPS | | QSISSYL |
| | | | TAVYYCARGDCDSTSCYR | | GLEWMGWI | | RFSGSGSGTDFT | | NWYQQ |
| | | | YSYGYEDYWGQGTLVTV | | NTDTGDPTY | | LTISSLQPEDFAT | | KPGKAP |
| | | | SSASTKGPSVFPLAPSSKS | | AQGFTGRFV | | YYCQQSYSTPPT | | KLLIYA |
| | | | TSGGTAALGCLVKDYFPEP | | FSLDTSVST | | FGQGTKVEIKRT | | ASSLQS |
| | | | VTVSWNSGALTSGVHTFP | | AFLQINSLK | | VAAPSVEIFPPS | | GVPSRFS |
| | | | AVLQSSGLYSLSSVVTVPS | | AEDTAVYY | | DEQLKSGTASV | | GSGSGT |
| | | | SSLGTQTYICNVNHKPSNT | | CARGDCDST | | VCLLNNFYPRE | | DFTLTIS |
| | | | KVDKKVEPKSCDKTHTCP | | SCYRYSYGY | | AKVQWKVDNA | | SLQPEDF |
| | | | PCPAPELLGGPSVFLFPPK | | EDYWGQGT | | LQSGNSQESVTE | | ATYYCQ |
| | | | PKDTLMISRTPEVTCVVVD | | LVTVSS | | QDSKDSTYSLSS | | QSYSTPP |
| | | | VSHEDPEVKFNWYVDGV | | | | TLTLSKADYEK | | TFGQGT |
| | | | EVHNAKTKPREEQYNSTY | | | | HKVYACEVTHQ | | KVEIK |
| | | | RVVSVLTVLHQDWLNGK | | | | GLSSPVTKSFNR | | |
| | | | EYKCKVSNKALPAPIEKTI | | | | GEC | | |
| | | | SKAKGQPREPQVYTLPPSR | | | | | | |
| | | | DELTKNQVSLWCLVKGFY | | | | | | |
| | | | PSDIAVEWESNGQPENNY | | | | | | |
| | | | KTTPPVLDSDGSFFLYSKL | | | | | | |
| | | | TVDKSRWQQGNVFSCSV | | | | | | |
| | | | MHEALHNHYTQKSLSLSP | | | | | | |
| | | | GKGGSHHHHHH | | | | | | |
| EPI441 | TROP2 | 546 | QVQLQQSGSELKKPGASV | 547 | QVQLQQSGS | 548 | DIQLTQSPSSLSA | 549 | DIQLTQS |
| | | | KVSCKASGYTFTNYGMN | | ELKKPGASV | | SVGDRVSITCKA | | PSSLSAS |
| | | | WVKQAPGQGLKWMGWI | | KVSCKASG | | SQDVSIAVAWY | | VGDRVS |
| | | | NTYTGEPTYTDDFKGRFA | | YTFTNYGM | | QQKPGKAPKLLI | | ITCKASQ |
| | | | FSLDTSVSTAYLQISSLKA | | NWVKQAPG | | YSASYRYTGVP | | DVSIAV |
| | | | DDTAVYFCARGGFGSSYW | | QGLKWMG | | DRFSGSGSGTDF | | AWYQQ |
| | | | YFDVWGQGSLVTVSSAST | | WINTYTGEP | | TLTISSLQPEDFA | | KPGKAP |
| | | | KGPSVFPLAPSSKSTSGGT | | TYTDDFKGR | | VYYCQQHYITPL | | KLLIYSA |
| | | | AALGCLVKDYFPEPVTVS | | FAFSLDTSV | | TFGAGTKVEIKR | | SYRYTG |
| | | | WNSGALTSGVHTFPAVLQ | | STAYLQISSL | | TVAAPSVFIFPPS | | VPDRFS |
| | | | SSGLYSLSSVVTVPSSSLG | | KADDTAVY | | DEQLKSGTASV | | GSGSGT |
| | | | TQTYICNVNHKPSNTKVD | | FCARGGFGS | | VCLLNNFYPRE | | DFTLTIS |
| | | | KKVEPKSCDKTHTCPPCP | | SYWYFDVW | | AKVQWKVDNA | | SLQPEDF |
| | | | APELLGGPSVFLFPPKPKD | | GQGSLVTVS | | LQSGNSQESVTE | | AVYYCQ |
| | | | TLMISRTPEVTCVVVDVSH | | S | | QDSKDSTYSLSS | | QHYITPL |
| | | | EDPEVKFNWYVDGVEVH | | | | TLTLSKADYEK | | TFGAGT |
| | | | NAKTKPREEQYNSTYRVV | | | | HKVYACEVTHQ | | KVEIK |
| | | | SVLTVLHQDWLNGKEYK | | | | GLSSPVTKSFNR | | |
| | | | CKVSNKALPAPIEKTISKA | | | | GEC | | |
| | | | KGQPREPQVYTLPPSRDEL | | | | | | |
| | | | TKNQVSLWCLVKGFYPSD | | | | | | |
| | | | IAVEWESNGQPENNYKTT | | | | | | |
| | | | PPVLDSDGSFFLYSKLTVD | | | | | | |
| | | | KSRWQQGNVFSCSVMHE | | | | | | |
| | | | ALHNHYTQKSLSLSPGKG | | | | | | |
| | | | GSHHHHHH | | | | | | |
| EPI501 | CXCR7 | 550 | EVKLVESGGDLVQPGGSL | 551 | EVKLVESGG | 552 | DVLMTQTPLSLP | 553 | DVLMTQ |
| | | | KLSCATSGFTFSDYYMFW | | DLVQPGGSL | | VSLGDQASISCR | | TPLSLPV |
| | | | VRQTPEKRLEWVAYITNG | | KLSCATSGF | | SSHYIVHSDGNT | | SLGDQA |
| | | | GDRSYYSDTVTGRFIISRD | | TFSDYYMF | | YLEWYLQKPGQ | | SISCRSS |
| | | | NAKNTLYLQMSRLKSEDT | | WVRQTPEK | | SPKLLIYKVSNR | | HYIVHS |
| | | | AMYYCARQGNWAAWFV | | RLEWVAYIT | | FSGVPDRFSGSG | | DGNTYL |
| | | | YWGQGTLVTVSAAKTTPP | | NGGDRSYY | | SGTDFTLKISRV | | EWYLQK |
| | | | SVYPLAPGSAAQTNSMVT | | SDTVTGRFI | | EAEDLGIYYCFQ | | PGQSPK |
| | | | LGCLVKGYFPEPVTVTWN | | ISRDNAKNT | | GSHVPLTFGAGT | | LLIYKVS |
| | | | SGSLSSGVHTFPAVLQSDL | | LYLQMSRLK | | KLELKRADAAP | | NRFSGV |
| | | | YTLSSSVTVPSSTWPSETV | | SEDTAMYYC | | TVSIFPPSSEQLT | | PDRFSGS |
| | | | TCNVAHPASSTKVDKKIEP | | ARQGNWAA | | SGGASVVCFLN | | GSGTDF |
| | | | KSCDKTHTCPPCPAPELLG | | WFVYWGQG | | NFYPKDINVKW | | TLKISRV |
| | | | GPSVFLFPPKPKDTLMISR | | TLVTVSA | | KIDGSERQNGVL | | EAEDLGI |
| | | | TPEVTCVVVDVSHEDPEV | | | | NSWTDQDSKDS | | YYCFQG |
| | | | KFNWYVDGVEVHNAKTK | | | | TYSMSSTLTLTK | | SHVPLTF |
| | | | PREEQYNSTYRVVSVLTV | | | | DEYERHNSYTC | | GAGTKL |
| | | | LHQDWLNGKEYKCKVSN | | | | EATHKTSTSPIV | | ELK |
| | | | KALPAPIEKTISKAKGQPR | | | | KSFNRNEC | | |
| | | | EPQVYTLPPSRDELTKNQV | | | | | | |
| | | | SLWCLVKGFYPSDIAVEW | | | | | | |
| | | | ESNGQPENNYKTTPPVLDS | | | | | | |
| | | | DGSFFLYSKLTVDKSRWQ | | | | | | |
| | | | QGNVFSCSVMHEALHNH | | | | | | |
| | | | YTQKSLSLSPGKGGSHHH | | | | | | |
| | | | HHH | | | | | | |
| EPI508 | HER3 | 554 | EVQLVESGGGLVQPGGSL | 555 | EVQLVESGG | 556 | DIQMTQSPSSLS | 557 | DIQMTQ |
| | | | RLSCAASGFTLSGDWIHW | | GLVQPGGSL | | ASVGDRVTITCR | | SPSSLSA |
| | | | VRQAPGKGLEWVGEISAA | | RLSCAASGF | | ASQNIATDVAW | | SVGDRV |
| | | | GGYTDYADSVKGRFTISA | | TLSGDWIH | | YQQKPGKAPKL | | TITCRAS |
| | | | DTSKNTAYLQMNSLRAED | | WVRQAPGK | | LIYSASFLYSGV | | QNIATD |
| | | | TAVYYCARESRVSFEAAM | | GLEWVGEIS | | PSRFSGSGSGTD | | VAWYQ |
| | | | DYWGQGTLVTVSSASTKG | | AAGGYTDY | | FTLTISSLQPEDF | | QKPGKA |
| | | | PSVFPLAPSSKSTSGGTAA | | ADSVKGRFT | | ATYYCQQSEPEP | | PKLLIYS |
| | | | LGCLVKDYFPEPVTVSWN | | ISADTSKNT | | YTFGQGTKVEIK | | ASFLYS |
| | | | SGALTSGVHTFPAVLQSSG | | AYLQMNSL | | RTVAAPSVFIFPP | | GVPSRFS |
| | | | LYSLSSVVTVPSSSLGTQT | | RAEDTAVY | | SDEQLKSGTASV | | GSGSGT |
| | | | YICNVNHKPSNTKVDKKV | | YCARESRVS | | VCLLNNFYPRE | | DFTLTIS |
| | | | EPKSCDKTHTCPPCPAPEL | | FEAAMDYW | | AKVQWKVDNA | | SLQPEDF |
| | | | LGGPSVFLFPPKPKDTLMI | | GQGTLVTVS | | LQSGNSQESVTE | | ATYYCQ |
| | | | SRTPEVTCVVVDVSHEDP | | S | | QDSKDSTYSLSS | | QSEPEPY |
| | | | EVKFNWYVDGVEVHNAK | | | | TLTLSKADYEK | | TFGQGT |
| | | | TKPREEQYNSTYRVVSVL | | | | HKVYACEVTHQ | | KVEIK |
| | | | TVLHQDWLNGKEYKCKV | | | | GLSSPVTKSFNR | | |
| | | | SNKALPAPIEKTISKAKGQ | | | | GEC | | |
| | | | PREPQVYTLPPSRDELTKN | | | | | | |
| | | | QVSLWCLVKGFYPSDIAV | | | | | | |
| | | | EWESNGQPENNYKTTPPV | | | | | | |
| | | | LDSDGSFFLYSKLTVDKSR | | | | | | |
| | | | WQQGNVFSCSVMHEALH | | | | | | |
| | | | NHYTQKSLSLSPGK | | | | | | |
| EPI511 | CD71 | 558 | QVQLVQSGAEVKKPGASV | 559 | QVQLVQSG | 560 | DIQMTQSPSSLS | 561 | DIQMTQ |
| | | | KMSCKASGYTFTSYWMH | | AEVKKPGAS | | ASVGDRVTITCS | | SPSSLSA |
| | | | WVRQAPGQGLEWIGAIYP | | VKMSCKAS | | ASSSVYYMYWF | | SVGDRV |
| | | | GNSETGYAQKFQGRATLT | | GYTFTSYW | | QQKPGKAPKLW | | TITCSAS |
| | | | ADTSTSTAYMELSSLRSED | | MHWVRQAP | | IYSTSNLASGVP | | SSVYYM |
| | | | TAVYYCTRENWDPGFAF | | GQGLEWIG | | SRFSGSGSGTDY | | YWFQQ |
| | | | WGQGTLITVSSASTKGPSV | | AIYPGNSET | | TLTISSMQPEDF | | KPGKAP |
| | | | FPLAPSSKSTSGGTAALGC | | GYAQKFQG | | ATYYCQQRRNY | | KLWIYS |
| | | | LVKDYFPEPVTVSWNSGA | | RATLTADTS | | PYTFGQGTKLEI | | TSNLAS |
| | | | LTSGVHTFPAVLQSSGLYS | | TSTAYMELS | | KRTVAAPSVFIF | | GVPSRFS |
| | | | LSSVVTVPSSSLGTQTYIC | | SLRSEDTAV | | PPSDEQLKSGTA | | GSGSGT |
| | | | NVNHKPSNTKVDKKVEPK | | YYCTRENW | | SVVCLLNNFYPR | | DYTLTIS |
| | | | SCDKTHTCPPCPAPELLGG | | DPGFAFWG | | EAKVQWKVDN | | SMQPED |
| | | | PSVFLFPPKPKDTLMISRT | | QGTLITVSS | | ALQSGNSQESVT | | FATYYC |
| | | | PEVTCVVVDVSHEDPEVKF | | | | EQDSKDSTYSLS | | QQRRNY |
| | | | NWYVDGVEVHNAKTKPR | | | | STLTLSKADYEK | | PYTFGQ |
| | | | EEQYNSTYRVVSVLTVLH | | | | HKVYACEVTHQ | | GTKLEIK |
| | | | QDWLNGKEYKCKVSNKA | | | | GLSSPVTKSFNR | | |
| | | | LPAPIEKTISKAKGQPREP | | | | GEC | | |
| | | | QVYTLPPSRDELTKNQVSL | | | | | | |
| | | | WCLVKGFYPSDIAVEWES | | | | | | |
| | | | NGQPENNYKTTPPVLDSD | | | | | | |
| | | | GSFFLYSKLTVDKSRWQQ | | | | | | |
| | | | GNVFSCSVMHEALHNHYT | | | | | | |
| | | | QKSLSLSPGK | | | | | | |
| EPI682 | EGFR | 562 | EVQLVQSGAEVKKPGASV | 563 | EVQLVQSG | 564 | EIVMTQSPATLS | 565 | EIVMTQ |
| | | | KVSCKASGYTFTTYTIHW | | AEVKKPGAS | | VSPGERATLSCK | | SPATLSV |
| | | | VRQAPGQGLEWMGYINPR | | VKVSCKAS | | ASQNVGINVAW | | SPGERA |
| | | | SGYTEYNQKFQDRVTMTR | | GYTFTTYTI | | YQQKPGQAPRA | | TLSCKA |
| | | | DTSTSTVYMELSSLRSEDT | | HWVRQAPG | | LIYSASYRYSGIP | | SQNVGI |
| | | | AVYYCARSYEFWGQGTT | | QGLEWMGY | | ARFSGSGSGTEF | | NVAWY |
| | | | VTVSSASTKGPSVFPLAPS | | INPRSGYTE | | TLTISSLQSEDFA | | QQKPGQ |
| | | | SKSTSGGTAALGCLVKDY | | YNQKFQDR | | VYYCHQYKTYP | | APRALIY |
| | | | FPEPVTVSWNSGALTSGV | | VTMTRDTST | | YTFGGGTKLEIK | | SASYRY |
| | | | HTFPAVLQSSGLYSLSSVV | | STVYMELSS | | RTVAAPSVFIFPP | | SGIPARF |
| | | | TVPSSSLGTQTYICNVNHK | | LRSEDTAVY | | SDEQLKSGTASV | | SGSGSG |
| | | | PSNTKVDKKVEPKSCDKT | | YCARSYEF | | VCLLNNFYPRE | | TEFTLTI |
| | | | HTCPPCPAPELLGGPSVFL | | WGQGTTVT | | AKVQWKVDNA | | SSLQSED |
| | | | FPPKPKDTLMISRTPEVTC | | VSS | | LQSGNSQESVTE | | FAVYYC |
| | | | VVVDVSHEDPEVKFNWY | | | | QDSKDSTYSLSS | | HQYKTY |
| | | | VDGVEVHNAKTKPREEQY | | | | TLTLSKADYEK | | PYTFGG |
| | | | NSTYRVVSVLTVLHQDWL | | | | HKVYACEVTHQ | | GTKLEIK |
| | | | NGKEYKCKVSNKALPAPI | | | | GLSSPVTKSFNR | | |
| | | | EKTISKAKGQPREPQVYTL | | | | GEC | | |
| | | | PPSRDELTKNQVSLWCLV | | | | | | |
| | | | KGFYPSDIAVEWESNGQP | | | | | | |
| | | | ENNYKTTPPVLDSDGSFFL | | | | | | |
| | | | YSKLTVDKSRWQQGNVFS | | | | | | |
| | | | CSVMHEALHNHYTQKSLS | | | | | | |
| | | | LSPGKGGSHHHHHH | | | | | | |
| EPI683 | EGFR | 566 | AVQLVESGGGSVQPGRSM | 567 | AVQLVESG | 568 | DVVLTQTPVSLS | 569 | DVVLTQ |
| | | | RLSCAASGFTFSNYDMTW | | GGSVQPGRS | | VTVGDQASISCR | | TPVSLSV |
| | | | VRQAPTKGLEWVASITSD | | MRLSCAAS | | SSQSLEYSDGYS | | TVGDQA |
| | | | GGSTYSRDSVKGRFTISRD | | GFTFSNYDM | | YLEWYLQKPGQ | | SISCRSS |
| | | | NAKSTLYLQMDSLRSEDT | | TWVRQAPT | | SPQLLIYEVSSRF | | QSLEYS |
| | | | ATYYCTTDRGRYLPYYFD | | KGLEWVASI | | SGVPDRFIGSGS | | DGYSYL |
| | | | YWGQGVMVTVSSASTKG | | TSDGGSTYS | | GTDFTLKISRVE | | EWYLQK |
| | | | PSVFPLAPSSKSTSGGTAA | | RDSVKGRFT | | PEDLGVYYCFQ | | PGQSPQ |
| | | | LGCLVKDYFPEPVTVSWN | | ISRDNAKST | | AIHDPTFGAGTK | | LLIYEVS |
| | | | SGALTSGVHTFPAVLQSSG | | LYLQMDSL | | LELKRTVAAPSV | | SRFSGVP |
| | | | LYSLSSVVTVPSSSLGTQT | | RSEDTATYY | | FIFPPSDEQLKSG | | DRFIGSG |
| | | | YICNVNHKPSNTKVDKKV | | CTTDRGRYL | | TASVVCLLNNF | | SGTDFT |
| | | | EPKSCDKTHTCPPCPAPEL | | PYYFDYWG | | YPREAKVQWKV | | LKISRVE |
| | | | LGGPSVFLFPPKPKDTLMI | | QGVMVTVS | | DNALQSGNSQE | | PEDLGV |
| | | | SRTPEVTCVVVDVSHEDP | | S | | SVTEQDSKDSTY | | YYCFQA |
| | | | EVKFNWYVDGVEVHNAK | | | | SLSSTLTLSKAD | | IHDPTFG |
| | | | TKPREEQYNSTYRVVSVL | | | | YEKHKVYACEV | | AGTKLE |
| | | | TVLHQDWLNGKEYKCKV | | | | THQGLSSPVTKS | | LK |
| | | | SNKALPAPIEKTISKAKGQ | | | | FNRGEC | | |
| | | | PREPQVYTLPPSRDELTKN | | | | | | |
| | | | QVSLWCLVKGFYPSDIAV | | | | | | |
| | | | EWESNGQPENNYKTTPPV | | | | | | |
| | | | LDSDGSFFLYSKLTVDKSR | | | | | | |
| | | | WQQGNVFSCSVMHEALH | | | | | | |
| | | | NHYTQKSLSLSPGKGGSH | | | | | | |
| | | | HHHHH | | | | | | |
| EPI684 | EGFR | 570 | QVQLKESGPGLVQPSQTLS | 571 | QVQLKESGP | 572 | DTVLTQSPALA | 573 | DTVLTQ |
| | | | LTCTVSGFSLTTYSVHWV | | GLVQPSQTL | | VSPGERVTISCR | | SPALAV |
| | | | RQHSGKNLEWMGRMWT | | SLTCTVSGF | | ASESVSKLMHW | | SPGERV |
| | | | AGDTSYNSAFTSRLNIFRD | | SLTTYSVHW | | YQQRPGQQPQL | | TISCRAS |
| | | | TSKSQVFLKMNSLQTEDT | | VRQHSGKN | | LIYLTSHLASGV | | ESVSKL |
| | | | GTYYCARSSYTSGYPFDS | | LEWMGRM | | PARFSGSGSGTD | | MHWYQ |
| | | | WGQGVMVTVSSASTKGPS | | WTAGDTSY | | FTLTIDPVEADD | | QRPGQQ |
| | | | VFPLAPSSKSTSGGTAALG | | NSAFTSRLNI | | TATYYCQQSRN | | PQLLIYL |
| | | | CLVKDYFPEPVTVSWNSG | | FRDTSKSQV | | DPTFGAGTKLEL | | TSHLAS |
| | | | ALTSGVHTFPAVLQSSGLY | | FLKMNSLQT | | KRTVAAPSVFIF | | GVPARF |
| | | | SLSSVVTVPSSSLGTQTYI | | EDTGTYYC | | PPSDEQLKSGTA | | SGSGSG |
| | | | CNVNHKPSNTKVDKKVEPK | | ARSSYTSGY | | SVVCLLNNFYPR | | TDFTLTI |
| | | | SCDKTHTCPPCPAPELLGG | | PFDSWGQG | | EAKVQWKVDN | | DPVEAD |
| | | | PSVFLFPPKPKDTLMISRT | | VMVTVSS | | ALQSGNSQESVT | | DTATYY |
| | | | PEVTCVVVDVSHEDPEVKF | | | | EQDSKDSTYSLS | | CQQSRN |
| | | | NWYVDGVEVHNAKTKPR | | | | STLTLSKADYEK | | DPTFGA |
| | | | EEQYNSTYRVVSVLTVLH | | | | HKVYACEVTHQ | | GTKLEL |
| | | | QDWLNGKEYKCKVSNKA | | | | GLSSPVTKSFNR | | K |
| | | | LPAPIEKTISKAKGQPREP | | | | GEC | | |
| | | | QVYTLPPSRDELTKNQVSL | | | | | | |
| | | | WCLVKGFYPSDIAVEWES | | | | | | |
| | | | NGQPENNYKTTPPVLDSD | | | | | | |
| | | | GSFFLYSKLTVDKSRWQQ | | | | | | |
| | | | GNVFSCSVMHEALHNHYT | | | | | | |
| | | | QKSLSLSPGKGGSHHHHH | | | | | | |
| | | | H | | | | | | |
| EPI685 | EGFR | 574 | EVQLVESGGGLVPPGKSL | 575 | EVQLVESGG | 576 | DIVMTQSPFSLA | 577 | DIVMTQ |
| | | | KLSCSASGFPFSNYGMHW | | GLVPPGKSL | | VSEGDMVTIMC | | SPFSLAV |
| | | | IRQAPGKGLDWVGYISSNS | | KLSCSASGF | | RSSQSLLSSGNQ | | SEGDMV |
| | | | GTIYADAVKGRFTISRDNA | | PFSNYGMH | | KNYLAWYQQK | | TIMCRSS |
| | | | KNTLYLLINSLKSEDTAM | | WIRQAPGK | | PGQSPKLLIYHA | | QSLLSSG |
| | | | YYCARGYFDGYYRFWGQ | | GLDWVGYIS | | STRQSGVPDRFI | | NQKNYL |
| | | | GVMVTVSSASTKGPSVFP | | SNSGTIYAD | | GSGSGTDFTLTI | | AWYQQ |
| | | | LAPSSKSTSGGTAALGCLV | | AVKGRFTIS | | SDVQAEDLADY | | KPGQSP |
| | | | KDYFPEPVTVSWNSGALT | | RDNAKNTL | | YCLQHYSSPTFG | | KLLIYH |
| | | | SGVHTFPAVLQSSGLYSLS | | YLLINSLKSE | | SGTKLEIKRTVA | | ASTRQS |
| | | | SVVTVPSSSLGTQTYICNV | | DTAMYYCA | | APSVFIFPPSDEQ | | GVPDRFI |
| | | | NHKPSNTKVDKKVEPKSC | | RGYFDGYY | | LKSGTASVVCLL | | GSGSGT |
| | | | DKTHTCPPCPAPELLGGPS | | RFWGQGVM | | NNFYPREAKVQ | | DFTLTIS |
| | | | VFLFPPKPKDTLMISRTPE | | VTVSS | | WKVDNALQSG | | DVQAED |
| | | | VTCVVVDVSHEDPEVKFN | | | | NSQESVTEQDSK | | LADYYC |
| | | | WYVDGVEVHNAKTKPRE | | | | DSTYSLSSTLTL | | LQHYSS |
| | | | EQYNSTYRVVSVLTVLHQ | | | | SKADYEKHKVY | | PTFGSGT |
| | | | DWLNGKEYKCKVSNKAL | | | | ACEVTHQGLSSP | | KLEIK |
| | | | PAPIEKTISKAKGQPREPQ | | | | VTKSFNRGEC | | |
| | | | VYTLPPSRDELTKNQVSL | | | | | | |
| | | | WCLVKGFYPSDIAVEWES | | | | | | |
| | | | NGQPENNYKTTPPVLDSD | | | | | | |
| | | | GSFFLYSKLTVDKSRWQQ | | | | | | |
| | | | GNVFSCSVMHEALHNHYT | | | | | | |
| | | | QKSLSLSPGKGGSHHHHH | | | | | | |
| | | | H | | | | | | |
| EPI686 | EGFR | 578 | EVHLVESGGGLVQPGGSL | 579 | EVHLVESGG | 580 | DTVLTQSPALTV | 581 | DTVLTQ |
| | | | KLSCAASGFTFSNYDMAW | | GLVQPGGSL | | SPGDKITISCRAS | | SPALTVS |
| | | | VRQAPTRGLEWVASISPG | | KLSCAASGF | | EGVNTRIHWYQ | | PGDKITI |
| | | | GGKTYYRDSVKGRLTISR | | TFSNYDMA | | QKSGQQPKLLIY | | SCRASE |
| | | | NNAENTQYLQIDSLRSEDT | | WVRQAPTR | | GASNLDSGVPD | | GVNTRI |
| | | | ATYYCSRLGPAYSGEWFA | | GLEWVASIS | | RFSGSGFGTDFT | | HWYQQ |
| | | | YWGQGTLVTVSSASTKGP | | PGGGKTYY | | LTIDPVEASDTA | | KSGQQP |
| | | | SVFPLAPSSKSTSGGTAAL | | RDSVKGRLT | | TYFCQQSWNVP | | KLLIYG |
| | | | GCLVKDYFPEPVTVSWNS | | ISRNNAENT | | HTFGGGTKLEL | | ASNLDS |
| | | | GALTSGVHTFPAVLQSSGL | | QYLQIDSLR | | KRTVAAPSVFIF | | GVPDRF |
| | | | YSLSSVVTVPSSSLGTQTY | | SEDTATYYC | | PPSDEQLKSGTA | | SGSGFG |
| | | | ICNVNHKPSNTKVDKKVEP | | SRLGPAYSG | | SVVCLLNNFYPR | | TDFTLTI |
| | | | KSCDKTHTCPPCPAPELLG | | EWFAYWGQ | | EAKVQWKVDN | | DPVEAS |
| | | | GPSVFLFPPKPKDTLMISR | | GTLVTVSS | | ALQSGNSQESVT | | DTATYF |
| | | | TPEVTCVVVDVSHEDPEV | | | | EQDSKDSTYSLS | | CQQSWN |
| | | | KFNWYVDGVEVHNAKTK | | | | STLTLSKADYEK | | VPHTFG |
| | | | PREEQYNSTYRVVSVLTV | | | | HKVYACEVTHQ | | GGTKLE |
| | | | LHQDWLNGKEYKCKVSN | | | | GLSSPVTKSFNR | | LK |
| | | | KALPAPIEKTISKAKGQPR | | | | GEC | | |
| | | | EPQVYTLPPSRDELTKNQV | | | | | | |
| | | | SLWCLVKGFYPSDIAVEW | | | | | | |
| | | | ESNGQPENNYKTTPPVLDS | | | | | | |
| | | | DGSFFLYSKLTVDKSRWQ | | | | | | |
| | | | QGNVFSCSVMHEALHNH | | | | | | |
| | | | YTQKSLSLSPGKGGSHHH | | | | | | |
| | | | HHH | | | | | | |
| EPI687 | EGFR | 582 | EVQLAESGGGLEQPGRSL | 583 | EVQLAESGG | 584 | DIVLTQSPALAV | 585 | DIVLTQS |
| | | | KLSCAASGFTFSNYDMAW | | GLEQPGRSL | | SLGQRATISCRA | | PALAVS |
| | | | VRQAPTKGLEWVASIIKSG | | KLSCAASGF | | SQSVTISGFNLM | | LGQRAT |
| | | | DTSYYRDSVKGRFTVSRD | | TFSNYDMA | | HWYQQKPGQQP | | ISCRASQ |
| | | | NAKSTLYLQMDSLRSEDT | | WVRQAPTK | | KLLIYRASNLAF | | SVTISGF |
| | | | ATYYCARHGVGSYDWFA | | GLEWVASII | | GIPARFSGSGSG | | NLMHW |
| | | | DWGQGTLVTVSSASTKGP | | KSGDTSYYR | | TDFTLTINPVQA | | YQQKPG |
| | | | SVFPLAPSSKSTSGGTAAL | | DSVKGRFTV | | DDFTTYYCQQS | | QQPKLLI |
| | | | GCLVKDYFPEPVTVSWNS | | SRDNAKSTL | | RKSRTFGGGTK | | YRASNL |
| | | | GALTSGVHTFPAVLQSSGL | | YLQMDSLRS | | LELKRTVAAPSV | | AFGIPAR |
| | | | YSLSSVVTVPSSSLGTQTY | | EDTATYYC | | FIFPPSDEQLKSG | | FSGSGS |
| | | | ICNVNHKPSNTKVDKKVEP | | ARHGVGSY | | TASVVCLLNNF | | GTDFTL |
| | | | KSCDKTHTCPPCPAPELLG | | DWFADWGQ | | YPREAKVQWKV | | TINPVQ |
| | | | GPSVFLFPPKPKDTLMISR | | GTLVTVSS | | DNALQSGNSQE | | ADDFTT |
| | | | TPEVTCVVVDVSHEDPEV | | | | SVTEQDSKDSTY | | YYCQQS |
| | | | KFNWYVDGVEVHNAKTK | | | | SLSSTLTLSKAD | | RKSRTF |
| | | | PREEQYNSTYRVVSVLTV | | | | YEKHKVYACEV | | GGGTKL |
| | | | LHQDWLNGKEYKCKVSN | | | | THQGLSSPVTKS | | ELK |
| | | | KALPAPIEKTISKAKGQPR | | | | FNRGEC | | |
| | | | EPQVYTLPPSRDELTKNQV | | | | | | |
| | | | SLWCLVKGFYPSDIAVEW | | | | | | |
| | | | ESNGQPENNYKTTPPVLDS | | | | | | |
| | | | DGSFFLYSKLTVDKSRWQ | | | | | | |
| | | | QGNVFSCSVMHEALHNH | | | | | | |
| | | | YTQKSLSLSPGKGGSHHH | | | | | | |
| | | | HHH | | | | | | |
| EPI688 | EGFR | 586 | EVQLVQSGSKLKKPGASV | 587 | EVQLVQSGS | 588 | DIQMTQSPSSLS | 589 | DIQMTQ |
| | | | KVSCKASGYTFTSYTMNW | | KLKKPGASV | | ASVGDRVTITCR | | SPSSLSA |
| | | | VRQAPGQGLEWMGWINT | | KVSCKASG | | ASQSISSYLNWY | | SVGDRV |
| | | | DTGDPTYAQGFTGRFVFS | | YTFTSYTMN | | QQKPGKAPKLLI | | TITCRAS |
| | | | LDTSVSTAFLQINSLKAED | | WVRQAPGQ | | YAASSLQSGVPS | | QSISSYL |
| | | | TAVYYCARGDCDSTSCYR | | GLEWMGWI | | RFSGSGSGTDFT | | NWYQQ |
| | | | YSYGYEDYWGQGTLVTV | | NTDTGDPTY | | LTISSLQPEDFAT | | KPGKAP |
| | | | SSASTKGPSVFPLAPSSKS | | AQGFTGRFV | | YYCQQSYSTPPT | | KLLIYA |
| | | | TSGGTAALGCLVKDYFPEP | | FSLDTSVST | | FGQGTKVEIKRT | | ASSLQS |
| | | | VTVSWNSGALTSGVHTFP | | AFLQINSLK | | VAAPSVFIFPPSD | | GVPSRFS |
| | | | AVLQSSGLYSLSSVVTVPS | | AEDTAVYY | | EQLKSGTASVV | | GSGSGT |
| | | | SSLGTQTYICNVNHKPSNT | | CARGDCDST | | CLLNNFYPREA | | DFTLTIS |
| | | | KVDKKVEPKSCDKTHTCP | | SCYRYSYGY | | KVQWKVDNAL | | SLQPEDF |
| | | | PCPAPELLGGPSVFLFPPK | | EDYWGQGT | | QSGNSQESVTEQ | | ATYYCQ |
| | | | PKDTLMISRTPEVTCVVVD | | LVTVSS | | DSKDSTYSLSST | | QSYSTPP |
| | | | VSHEDPEVKFNWYVDGV | | | | LTLSKADYEKH | | TFGQGT |
| | | | EVHNAKTKPREEQYNSTY | | | | KVYACEVTHQG | | KVEIK |
| | | | RVVSVLTVLHQDWLNGK | | | | LSSPVTKSFNRG | | |
| | | | EYKCKVSNKALPAPIEKTI | | | | EC | | |
| | | | SKAKGQPREPQVYTLPPSR | | | | | | |
| | | | DELTKNQVSLWCLVKGFY | | | | | | |
| | | | PSDIAVEWESNGQPENNY | | | | | | |
| | | | KTTPPVLDSDGSFFLYSKL | | | | | | |
| | | | TVDKSRWQQGNVFSCSV | | | | | | |
| | | | MHEALHNHYTQKSLSLSP | | | | | | |
| | | | GKGGSHHHHHH | | | | | | |
| EPI689 | EGFR | 590 | EVQLVESGGGLVQPGGSL | 591 | EVQLVESGG | 592 | DIQMTQSPSSLS | 593 | DIQMTQ |
| | | | RLSCVVSGFTFSYYDMHW | | GLVQPGGSL | | ASVGDRVTITCR | | SPSSLSA |
| | | | VRQVTGKGLEWVSAIGTA | | RLSCVVSGF | | ASQSISSYLNWY | | SVGDRV |
| | | | GATYYPGSVKGRFTISREN | | TFSYYDMH | | QQKPGKAPKLLI | | TITCRAS |
| | | | AKNSLYLQMNSLRAGDTA | | WVRQVTGK | | YAASSLQSGVPS | | QSISSYL |
| | | | VYYCARDRGYSGYDAYY | | GLEWVSAIG | | RFSGSGSGTDFT | | NWYQQ |
| | | | FDFWGQGTLVTVSSASTK | | TAGATYYP | | LTISSLQPEDFAT | | KPGKAP |
| | | | GPSVFPLAPSSKSTSGGTA | | GSVKGRFTI | | YYCQQSYSTPPT | | KLLIYA |
| | | | ALGCLVKDYFPEPVTVSW | | SRENAKNSL | | FGQGTKVEIKRT | | ASSLQS |
| | | | NSGALTSGVHTFPAVLQSS | | YLQMNSLR | | VAAPSVFIFPPSD | | GVPSRFS |
| | | | GLYSLSSVVTVPSSSLGTQ | | AGDTAVYY | | EQLKSGTASVV | | GSGSGT |
| | | | TYICNVNHKPSNTKVDKK | | CARDRGYS | | CLLNNFYPREA | | DFTLTIS |
| | | | VEPKSCDKTHTCPPCPAPE | | GYDAYYFD | | KVQWKVDNAL | | SLQPEDF |
| | | | LLGGPSVFLFPPKPKDTLM | | FWGQGTLV | | QSGNSQESVTEQ | | ATYYCQ |
| | | | ISRTPEVTCVVVDVSHEDP | | TVSS | | DSKDSTYSLSST | | QSYSTPP |
| | | | EVKFNWYVDGVEVHNAK | | | | LTLSKADYEKH | | TFGQGT |
| | | | TKPREEQYNSTYRVVSVL | | | | KVYACEVTHQG | | KVEIK |
| | | | TVLHQDWLNGKEYKCKV | | | | LSSPVTKSFNRG | | |
| | | | SNKALPAPIEKTISKAKGQ | | | | EC | | |
| | | | PREPQVYTLPPSRDELTKN | | | | | | |
| | | | QVSLWCLVKGFYPSDIAV | | | | | | |
| | | | EWESNGQPENNYKTTPPV | | | | | | |
| | | | LDSDGSFFLYSKLTVDKSR | | | | | | |
| | | | WQQGNVFSCSVMHEALH | | | | | | |
| | | | NHYTQKSLSLSPGKGGSH | | | | | | |
| | | | HHHHH | | | | | | |
| EPI690 | EGFR | 594 | QVQLQESGPGLVKPSETLS | 595 | QVQLQESGP | 596 | DIQMTQSPSSLS | 597 | DIQMTQ |
| | | | LTCTVSGGSISSSNYYWG | | GLVKPSETL | | ASVGDRVTITCR | | SPSSLSA |
| | | | WIRQPPGKGLEWIGNIYYR | | SLTCTVSGG | | ASQSISSYLNWY | | SVGDRV |
| | | | GYTYYNPSLKSRVTISVDT | | SISSSNYYW | | QQKPGKAPKLLI | | TITCRAS |
| | | | SKKQFSLTLSSVTAADTA | | GWIRQPPGK | | YAASSLQSGVPS | | QSISSYL |
| | | | MYYCAREGSDYGDYVGA | | GLEWIGNIY | | RFSGSGSGTDFT | | NWYQQ |
| | | | FDIWDQGTMVTVSSASTK | | YRGYTYYN | | LTISSLQPEDFAT | | KPGKAP |
| | | | GPSVFPLAPSSKSTSGGTA | | PSLKSRVTIS | | YYCQQSYSTPPT | | KLLIYA |
| | | | ALGCLVKDYFPEPVTVSW | | VDTSKKQFS | | FGQGTKVEIKRT | | ASSLQS |
| | | | NSGALTSGVHTFPAVLQSS | | LTLSSVTAA | | VAAPSVFIFPPSD | | GVPSRFS |
| | | | GLYSLSSVVTVPSSSLGTQ | | DTAMYYCA | | EQLKSGTASVV | | GSGSGT |
| | | | TYICNVNHKPSNTKVDKK | | REGSDYGD | | CLLNNFYPREA | | DFTLTIS |
| | | | VEPKSCDKTHTCPPCPAPE | | YVGAFDIW | | KVQWKVDNAL | | SLQPEDF |
| | | | LLGGPSVFLFPPKPKDTLM | | DQGTMVTV | | QSGNSQESVTEQ | | ATYYCQ |
| | | | ISRTPEVTCVVVDVSHEDP | | SS | | DSKDSTYSLSST | | QSYSTPP |
| | | | EVKFNWYVDGVEVHNAK | | | | LTLSKADYEKH | | TFGQGT |
| | | | ITKPREEQYNSTYRVVSVL | | | | KVYACEVTHQG | | KVEIK |
| | | | TVLHQDWLNGKEYKCKV | | | | LSSPVTKSFNRG | | |
| | | | SNKALPAPIEKTISKAKGQ | | | | EC | | |
| | | | PREPQVYTLPPSRDELTKN | | | | | | |
| | | | QVSLWCLVKGFYPSDIAV | | | | | | |
| | | | EWESNGQPENNYKTTPPV | | | | | | |
| | | | LDSDGSFFLYSKLTVDKSR | | | | | | |
| | | | WQQGNVFSCSVMHEALH | | | | | | |
| | | | NHYTQKSLSLSPGKGGSH | | | | | | |
| | | | HHHHH | | | | | | |
| EPI691 | EGFR | 598 | EVQLVQSGGGLVQPGGSL | 599 | EVQLVQSG | 600 | DIQMTQSPSSLS | 601 | DIQMTQ |
| | | | RLSCAASGFTFSYYDMHW | | GGLVQPGGS | | ASVGDRVTITCR | | SPSSLSA |
| | | | VRQVTGKGLEWVSTIGAT | | LRLSCAASG | | ASQSISSYLNWY | | SVGDRV |
| | | | GDTYYSDSVKGRFTISRQN | | FTFSYYDMH | | QQKPGKAPKLLI | | TITCRAS |
| | | | AKNSLYLQINSLRAGDTA | | WVRQVTGK | | YAASSLQSGVPS | | QSISSYL |
| | | | VYYCVRDRGYIGYDSYYF | | GLEWVSTIG | | RFSGSGSGTDFT | | NWYQQ |
| | | | DNWGQGTLVTVSSASTKG | | ATGDTYYS | | LTISSLQPEDFAT | | KPGKAP |
| | | | PSVFPLAPSSKSTSGGTAA | | DSVKGRFTI | | YYCQQSYSTPPT | | KLLIYA |
| | | | LGCLVKDYFPEPVTVSWN | | SRQNAKNSL | | FGQGTKVEIKRT | | ASSLQS |
| | | | SGALTSGVHTFPAVLQSSG | | YLQINSLRA | | VAAPSVFIFPPSD | | GVPSRFS |
| | | | LYSLSSVVTVPSSSLGTQT | | GDTAVYYC | | EQLKSGTASVV | | GSGSGT |
| | | | YICNVNHKPSNTKVDKKV | | VRDRGYIGY | | CLLNNFYPREA | | DFTLTIS |
| | | | EPKSCDKTHTCPPCPAPEL | | DSYYFDNW | | KVQWKVDNAL | | SLQPEDF |
| | | | LGGPSVFLFPPKPKDTLMI | | GQGTLVTVS | | QSGNSQESVTEQ | | ATYYCQ |
| | | | SRTPEVTCVVVDVSHEDP | | S | | DSKDSTYSLSST | | QSYSTPP |
| | | | EVKFNWYVDGVEVHNAK | | | | LTLSKADYEKH | | TFGQGT |
| | | | TKPREEQYNSTYRVVSVL | | | | KVYACEVTHQG | | KVEIK |
| | | | TVLHQDWLNGKEYKCKV | | | | LSSPVTKSFNRG | | |
| | | | SNKALPAPIEKTISKAKGQ | | | | EC | | |
| | | | PREPQVYTLPPSRDELTKN | | | | | | |
| | | | QVSLWCLVKGFYPSDIAV | | | | | | |
| | | | EWESNGQPENNYKTTPPV | | | | | | |
| | | | LDSDGSFFLYSKLTVDKSR | | | | | | |
| | | | WQQGNVFSCSVMHEALH | | | | | | |
| | | | NHYTQKSLSLSPGKGGSH | | | | | | |
| | | | HHHHH | | | | | | |
| EPI704 | MUC1 | 602 | EVQLVESGGGLVQPGGSL | 603 | EVQLVESGG | 604 | DIVMTQSPLSNP | 605 | DIVMTQ |
| | | | RLSCAASGFNIKDTYIHWV | | GLVQPGGSL | | VTPGEPASISCRS | | SPLSNPV |
| | | | RQAPGKGLEWVARIYPTN | | RLSCAASGF | | SKSLLHSNGITY | | TPGEPAS |
| | | | GYTRYADSVKGRETISAD | | NIKDTYIHW | | FFWYLQKPGQS | | SCRSSK |
| | | | TSKNTAYLQMNSLRAEDT | | VRQAPGKG | | PQLLIYQMSNLA | | SLLHSN |
| | | | AVYYCSRWGGDGFYAMD | | LEWVARIYP | | SGVPDRFSGSGS | | GITYFF |
| | | | YWGQGTLVTVSSASTKGP | | TNGYTRYA | | GTDFTLRISRVE | | WYLQKP |
| | | | SVFPLAPSSKSTSGGTAAL | | DSVKGRETI | | AEDVGVYYCAQ | | GQSPQL |
| | | | GCLVKDYFPEPVTVSWNS | | SADTSKNTA | | NLELPPTFGQGT | | LIYQMS |
| | | | GALTSGVHTFPAVLQSSGL | | YLQMNSLR | | KVEIKRTVAAPS | | NLASGV |
| | | | YSLSSVVTVPSSSLGTQTY | | AEDTAVYY | | VFIFPPSDEQLKS | | PDRFSGS |
| | | | ICNVNHKPSNTKVDKKVEP | | CSRWGGDG | | GTASVVCLLNN | | GSGTDF |
| | | | KSCDKTHTCPPCPAPELLG | | FYAMDYWG | | FYPREAKVQWK | | TLRISRV |
| | | | GPSVFLFPPKPKDTLMISR | | QGTLVTVSS | | VDNALQSGNSQ | | EAEDVG |
| | | | TPEVTCVVVDVSHEDPEV | | | | ESVTEQDSKDST | | VYYCAQ |
| | | | KFNWYVDGVEVHNAKTK | | | | YSLSSTLTLSKA | | NLELPPT |
| | | | PREEQYNSTYRVVSVLTV | | | | DYEKHKVYACE | | FGQGTK |
| | | | LHQDWLNGKEYKCKVSN | | | | VTHQGLSSPVTK | | VEIK |
| | | | KALPAPIEKTISKAKGQPR | | | | SFNRGEC | | |
| | | | EPQVYTLPPSRDELTKNQV | | | | | | |
| | | | SLWCLVKGFYPSDIAVEW | | | | | | |
| | | | ESNGQPENNYKTTPPVLDS | | | | | | |
| | | | DGSFFLYSKLTVDKSRWQ | | | | | | |
| | | | QGNVFSCSVMHEALHNH | | | | | | |
| | | | YTQKSLSLSPGK | | | | | | |
| EPI705 | MUC16 | 606 | QVQLVESGGGLVKPGGSL | 607 | QVQLVESG | 608 | DIQMTQSPSSLS | 609 | DIQMTQ |
| | | | RLSCAASGFTFSNYYMSW | | GGLVKPGGS | | ASVGDRVTITCR | | SPSSLSA |
| | | | VRQAPGKGLEWISYISGRG | | LRLSCAASG | | ASQSISTYLNWY | | SVGDRV |
| | | | STIFYADSVKGRITISRDN | | FTFSNYYMS | | QQKPGKAPKLLI | | TITCRAS |
| | | | AKNSLFLQMNSLRAEDTAV | | WVRQAPGK | | YTASSLQSGVPS | | QSISTYL |
| | | | YFCVKDRGGYSPYWGQG | | GLEWISYIS | | RFSGSGSGTDFT | | NWYQQ |
| | | | TLVTVSSASTKGPSVFPLA | | GRGSTIFYA | | LTISSLQPEDFAT | | KPGKAP |
| | | | PSSKSTSGGTAALGCLVK | | DSVKGRITIS | | YYCQQSYSTPPI | | KLLIYTA |
| | | | DYFPEPVTVSWNSGALTS | | RDNAKNSLF | | TFGQGTRLEIKR | | SSLQSG |
| | | | GVHTFPAVLQSSGLYSLSS | | LQMNSLRA | | TVAAPSVFIFPPS | | VPSRFSG |
| | | | VVTVPSSSLGTQTYICNVN | | EDTAVYFCV | | DEQLKSGTASV | | SGSGTD |
| | | | HKPSNTKVDKKVEPKSCD | | KDRGGYSP | | VCLLNNFYPRE | | FTLTISS |
| | | | KTHTCPPCPAPELLGGPSV | | YWGQGTLV | | AKVQWKVDNA | | LQPEDF |
| | | | FLFPPKPKDTLMISRTPEV | | TVSS | | LQSGNSQESVTE | | ATYYCQ |
| | | | TCVVVDVSHEDPEVKFNW | | | | QDSKDSTYSLSS | | QSYSTPP |
| | | | YVDGVEVHNAKTKPREEQ | | | | TLTLSKADYEK | | ITFGQGT |
| | | | YNSTYRVVSVLTVLHQD | | | | HKVYACEVTHQ | | RLEIK |
| | | | WLNGKEYKCKVSNKALP | | | | GLSSPVTKSFNR | | |
| | | | APIEKTISKAKGQPREPQV | | | | GEC | | |
| | | | YTLPPSRDELTKNQVSLW | | | | | | |
| | | | CLVKGFYPSDIAVEWESN | | | | | | |
| | | | GQPENNYKTTPPVLDSDG | | | | | | |
| | | | SFFLYSKLTVDKSRWQQG | | | | | | |
| | | | NVFSCSVMHEALHNHYTQ | | | | | | |
| | | | KSLSLSPGK | | | | | | |
| EPI706 | MUC16 | 610 | EVQLVESGGGLVQPGGSL | 611 | EVQLVESGG | 612 | DIQMTQSPSSLS | 613 | DIQMTQ |
| | | | RLSCAASGYSITNDYAWN | | GLVQPGGSL | | ASVGDRVTITCK | | SPSSLSA |
| | | | WVRQAPGKGLEWVGYIS | | RLSCAASGY | | ASDLIHNWLAW | | SVGDRV |
| | | | YSGYTTYNPSLKSRFTISR | | SITNDYAWN | | YQQKPGKAPKL | | TITCKAS |
| | | | DTSKNTLYLQMNSLRAED | | WVRQAPGK | | LIYGATSLETGV | | DLIHNW |
| | | | TAVYYCARWTSGLDYWG | | GLEWVGYIS | | PSRFSGSGSGTD | | LAWYQ |
| | | | QGTLVTVSSASTKGPSVFP | | YSGYTTYNP | | FTLTISSLQPEDF | | QKPGKA |
| | | | LAPSSKSTSGGTAALGCLV | | SLKSRFTIS | | ATYYCQQYWTT | | PKLLIYG |
| | | | KDYFPEPVTVSWNSGALT | | RDTSKNTLY | | PFTFGQGTKVEI | | ATSLET |
| | | | SGVHTFPAVLQSSGLYSLS | | QMNSLRAE | | KRTVAAPSVFIF | | GVPSRFS |
| | | | SVVTVPSSSLGTQTYICNV | | LDTAVYYCA | | PPSDEQLKSGTA | | GSGSGT |
| | | | NHKPSNTKVDKKVEPKSC | | RWTSGLDY | | SVVCLLNNFYPR | | DFTLTIS |
| | | | DKTHTCPPCPAPELLGGPS | | WGQGTLVT | | EAKVQWKVDN | | SLQPEDF |
| | | | VFLFPPKPKDTLMISRTPE | | VSS | | ALQSGNSQESVT | | ATYYCQ |
| | | | VTCVVVDVSHEDPEVKFN | | | | EQDSKDSTYSLS | | QYWTTP |
| | | | WYVDGVEVHNAKTKPRE | | | | STLTLSKADYEK | | FTFGQG |
| | | | EQYNSTYRVVSVLTVLHQ | | | | HKVYACEVTHQ | | TKVEIK |
| | | | DWLNGKEYKCKVSNKAL | | | | GLSSPVTKSFNR | | |
| | | | PAPIEKTISKAKGQPREPQ | | | | GEC | | |
| | | | VYTLPPSRDELTKNQVSL | | | | | | |
| | | | WCLVKGFYPSDIAVEWES | | | | | | |
| | | | NGQPENNYKTTPPVLDSD | | | | | | |
| | | | GSFFLYSKLTVDKSRWQQ | | | | | | |
| | | | GNVFSCSVMHEALHNHYT | | | | | | |
| | | | QKSLSLSPGK | | | | | | |
| EPI707 | TROP2 | 614 | QVQLQQSGSELKKPGASV | 615 | QVQLQQSGS | 616 | DIQLTQSPSSLSA | 617 | DIQLTQS |
| | | | KVSCKASGYTFTNYGMN | | ELKKPGASV | | SVGDRVSITCKA | | PSSLSAS |
| | | | WVKQAPGQGLKWMGWI | | KVSCKASG | | SQDVSIAVAWY | | VGDRVS |
| | | | NTYTGEPTYTDDFKGRFA | | YTFTNYGM | | QQKPGKAPKLLI | | ITCKASQ |
| | | | FSLDTSVSTAYLQISSLKA | | NWVKQAPG | | YSASYRYTGVP | | DVSIAV |
| | | | DDTAVYFCARGGFGSSYW | | QGLKWMG | | DRFSGSGSGTDF | | AWYQQ |
| | | | YFDVWGQGSLVTVSSAST | | WINTYTGEP | | TLTISSLQPEDFA | | KPGKAP |
| | | | KGPSVFPLAPSSKSTSGGT | | TYTDDFKGR | | VYYCQQHYITPL | | KLLIYSA |
| | | | AALGCLVKDYFPEPVTVS | | FAFSLDTSV | | TFGAGTKVEIKR | | SYRYTG |
| | | | WNSGALTSGVHTFPAVLQ | | STAYLQISSL | | TVAAPSVFIFPPS | | VPDRFS |
| | | | SSGLYSLSSVVTVPSSSLG | | KADDTAVY | | DEQLKSGTASV | | GSGSGT |
| | | | TQTYICNVNHKPSNTKVD | | FCARGGFGS | | VCLLNNFYPRE | | DFTLTIS |
| | | | KKVEPKSCDKTHTCPPCP | | SYWYFDVW | | AKVQWKVDNA | | SLQPEDF |
| | | | APELLGGPSVFLFPPKPKD | | GQGSLVTVS | | LQSGNSQESVTE | | AVYYCQ |
| | | | TLMISRTPEVTCVVVDVSH | | S | | QDSKDSTYSLSS | | QHYITPL |
| | | | EDPEVKFNWYVDGVEVH | | | | TLTLSKADYEK | | TFGAGT |
| | | | NAKTKPREEQYNSTYRVV | | | | HKVYACEVTHQ | | KVEIK |
| | | | SVLTVLHQDWLNGKEYK | | | | GLSSPVTKSFNR | | |
| | | | CKVSNKALPAPIEKTISKA | | | | GEC | | |
| | | | KGQPREPQVYTLPPSRDEL | | | | | | |
| | | | TKNQVSLWCLVKGFYPSD | | | | | | |
| | | | IAVEWESNGQPENNYKTT | | | | | | |
| | | | PPVLDSDGSFFLYSKLTVD | | | | | | |
| | | | KSRWQQGNVFSCSVMHE | | | | | | |
| | | | ALHNHYTQKSLSLSPGK | | | | | | |
| EPI708 | RNF43 | 618 | QVQLQESGPGLVKPSETLS | 619 | QVQLQESGP | 620 | DIQMTQSPSSLS | 621 | DIQMTQ |
| | | | LTCTVSGGSISSSNYYWG | | GLVKPSETL | | ASVGDRVTITCR | | SPSSLSA |
| | | | WIRQPPGKGLEWIGNIYYR | | SLTCTVSGG | | ASQSISSYLNWY | | SVGDRV |
| | | | GYTYYNPSLKSRVTISVDT | | SISSSNYYW | | QQKPGKAPKLLI | | TITCRAS |
| | | | SKKQFSLTLSSVTAADTA | | GWIRQPPGK | | YAASSLQSGVPS | | QSISSYL |
| | | | MYYCAREGSDYGDYVGA | | GLEWIGNIY | | RFSGSGSGTDFT | | NWYQQ |
| | | | FDIWDQGTMVTVSSASTK | | YRGYTYYN | | LTISSLQPEDFAT | | KPGKAP |
| | | | GPSVFPLAPSSKSTSGGTA | | PSLKSRVTIS | | YYCQQSYSTPPT | | KLLIYA |
| | | | ALGCLVKDYFPEPVTVSW | | VDTSKKQFS | | FGQGTKVEIKRT | | ASSLQS |
| | | | NSGALTSGVHTFPAVLQSS | | LTLSSVTAA | | VAAPSVFIFPPSD | | GVPSRFS |
| | | | GLYSLSSVVTVPSSSLGTQ | | DTAMYYCA | | EQLKSGTASVV | | GSGSGT |
| | | | TYICNVNHKPSNTKVDKK | | REGSDYGD | | CLLNNFYPREA | | DFTLTIS |
| | | | VEPKSCDKTHTCPPCPAPE | | YVGAFDIW | | KVQWKVDNAL | | SLQPEDF |
| | | | LLGGPSVFLFPPKPKDTLM | | DQGTMVTV | | QSGNSQESVTEQ | | ATYYCQ |
| | | | ISRTPEVTCVVVDVSHEDP | | SS | | DSKDSTYSLSST | | QSYSTPP |
| | | | EVKFNWYVDGVEVHNAK | | | | LTLSKADYEKH | | TFGQGT |
| | | | TKPREEQYNSTYRVVSVL | | | | KVYACEVTHQG | | KVEIK |
| | | | TVLHQDWLNGKEYKCKV | | | | LSSPVTKSFNRG | | |
| | | | SNKALPAPIEKTISKAKGQ | | | | EC | | |
| | | | PREPQVYTLPPSRDELTKN | | | | | | |
| | | | QVSLWCLVKGFYPSDIAV | | | | | | |
| | | | EWESNGQPENNYKTTPPV | | | | | | |
| | | | LDSDGSFFLYSKLTVDKSR | | | | | | |
| | | | WQQGNVFSCSVMHEALH | | | | | | |
| | | | NHYTQKSLSLSPGK | | | | | | |
| EPI709 | RNF43 | 622 | EVQLVQSGAEVKKPGASV | 623 | EVQLVQSG | 624 | EIVMTQSPATLS | 625 | EIVMTQ |
| | | | KVSCKASGYTFTTYTIHW | | AEVKKPGAS | | VSPGERATLSCK | | SPATLSV |
| | | | VRQAPGQGLEWMGYINPR | | VKVSCKAS | | ASQNVGINVAW | | SPGERA |
| | | | SGYTEYNQKFQDRVTMTR | | GYTFTTYTI | | YQQKPGQAPRA | | TLSCKA |
| | | | DTSTSTVYMELSSLRSEDT | | HWVRQAPG | | LIYSASYRYSGIP | | SQNVGI |
| | | | AVYYCARSYEFWGQGTT | | QGLEWMGY | | ARFSGSGSGTEF | | NVAWY |
| | | | VTVSSASTKGPSVFPLAPS | | INPRSGYTE | | TLTISSLQSEDFA | | QQKPGQ |
| | | | SKSTSGGTAALGCLVKDY | | YNQKFQDR | | VYYCHQYKTYP | | APRALIY |
| | | | FPEPVTVSWNSGALTSGV | | VTMTRDTST | | YTFGGGTKLEIK | | SASYRY |
| | | | HTFPAVLQSSGLYSLSSVV | | STVYMELSS | | RTVAAPSVFIFPP | | SGIPARF |
| | | | TVPSSSLGTQTYICNVNHK | | LRSEDTAVY | | SDEQLKSGTASV | | SGSGSG |
| | | | PSNTKVDKKVEPKSCDKT | | YCARSYEF | | VCLLNNFYPRE | | TEFTLTI |
| | | | HTCPPCPAPELLGGPSVFL | | WGQGTTVT | | AKVQWKVDNA | | SSLQSED |
| | | | FPPKPKDTLMISRTPEVTC | | VSS | | LQSGNSQESVTE | | FAVYYC |
| | | | VVVDVSHEDPEVKFNWY | | | | QDSKDSTYSLSS | | HQYKTY |
| | | | VDGVEVHNAKTKPREEQY | | | | TLTLSKADYEK | | PYTFGG |
| | | | NSTYRVVSVLTVLHQDWL | | | | HKVYACEVTHQ | | GTKLEIK |
| | | | NGKEYKCKVSNKALPAPI | | | | GLSSPVTKSFNR | | |
| | | | EKTISKAKGQPREPQVYTL | | | | GEC | | |
| | | | PPSRDELTKNQVSLWCLV | | | | | | |
| | | | KGFYPSDIAVEWESNGQP | | | | | | |
| | | | ENNYKTTPPVLDSDGSFFL | | | | | | |
| | | | YSKLTVDKSRWQQGNVFS | | | | | | |
| | | | CSVMHEALHNHYTQKSLS | | | | | | |
| | | | LSPGK | | | | | | |
| EPI710 | RNF43 | 626 | AVQLVESGGGSVQPGRSM | 627 | AVQLVESG | 628 | DVVLTQTPVSLS | 629 | DVVLTQ |
| | | | RLSCAASGFTFSNYDMTW | | GGSVQPGRS | | VTVGDQASISCR | | TPVSLSV |
| | | | VRQAPTKGLEWVASITSD | | MRLSCAAS | | SSQSLEYSDGYS | | TVGDQA |
| | | | GGSTYSRDSVKGRFTISRD | | GFTFSNYDM | | YLEWYLQKPGQ | | SISCRSS |
| | | | NAKSTLYLQMDSLRSEDT | | TWVRQAPT | | SPQLLIYEVSSRF | | QSLEYS |
| | | | ATYYCTTDRGRYLPYYFD | | KGLEWVASI | | SGVPDRFIGSGS | | DGYSYL |
| | | | YWGQGVMVTVSSASTKG | | TSDGGSTYS | | GTDFTLKISRVE | | EWYLQK |
| | | | PSVFPLAPSSKSTSGGTAA | | RDSVKGRFT | | PEDLGVYYCFQ | | PGQSPQ |
| | | | LGCLVKDYFPEPVTVSWN | | ISRDNAKST | | AIHDPTFGAGTK | | LLIYEVS |
| | | | SGALTSGVHTFPAVLQSSG | | LYLQMDSL | | LELKRTVAAPSV | | SRFSGVP |
| | | | LYSLSSVVTVPSSSLGTQT | | RSEDTATYY | | FIFPPSDEQLKSG | | DRFIGSG |
| | | | YICNVNHKPSNTKVDKKV | | CTTDRGRYL | | TASVVCLLNNF | | SGTDFT |
| | | | EPKSCDKTHTCPPCPAPEL | | PYYFDYWG | | YPREAKVQWKV | | LKISRVE |
| | | | LGGPSVFLFPPKPKDTLMI | | QGVMVTVS | | DNALQSGNSQE | | PEDLGV |
| | | | SRTPEVTCVVVDVSHEDP | | S | | SVTEQDSKDSTY | | YYCFQA |
| | | | EVKFNWYVDGVEVHNAK | | | | SLSSTLTLSKAD | | IHDPTFG |
| | | | TKPREEQYNSTYRVVSVL | | | | YEKHKVYACEV | | AGTKLE |
| | | | TVLHQDWLNGKEYKCKV | | | | THQGLSSPVTKS | | LK |
| | | | SNKALPAPIEKTISKAKGQ | | | | FNRGEC | | |
| | | | PREPQVYTLPPSRDELTKN | | | | | | |
| | | | QVSLWCLVKGFYPSDIAV | | | | | | |
| | | | EWESNGQPENNYKTTPPV | | | | | | |
| | | | LDSDGSFFLYSKLTVDKSR | | | | | | |
| | | | WQQGNVFSCSVMHEALH | | | | | | |
| | | | NHYTQKSLSLSPGK | | | | | | |
| EPI711 | HER3 | 630 | EVQLLESGGGLVQPGGSL | 631 | EVQLLESGG | 632 | EVQLLESGGGL | 633 | EVQLLE |
| | | | RLSCAASGFTFSSYAMSW | | GLVQPGGSL | | VQPGGSLRLSCA | | SGGGLV |
| | | | VRQAPGKGLEWVSAINSQ | | RLSCAASGF | | ASGFTFSSYAMS | | QPGGSL |
| | | | GKSTYYADSVKGRFTISRD | | TFSSYAMS | | WVRQAPGKGLE | | RLSCAA |
| | | | NSKNTLYLQMNSLRAEDT | | WVRQAPGK | | WVSAINSQGKS | | SGFTFSS |
| | | | AVYYCARWGDEGFDIWG | | GLEWVSAIN | | TYYADSVKGRF | | YAMSW |
| | | | QGTLVTVSSASTKGPSVFP | | SQGKSTYYA | | TISRDNSKNTLY | | VRQAPG |
| | | | LAPSSKSTSGGTAALGCLV | | DSVKGRFTI | | LQMNSLRAEDT | | KGLEWV |
| | | | KDYFPEPVTVSWNSGALT | | SRDNSKNTL | | AVYYCARWGD | | SAINSQG |
| | | | SGVHTFPAVLQSSGLYSLS | | YLQMNSLR | | EGFDIWGQGTL | | KSTYYA |
| | | | SVVTVPSSSLGTQTYICNV | | AEDTAVYY | | VTVSSRTVAAPS | | DSVKGR |
| | | | NHKPSNTKVDKKVEPKSC | | CARWGDEG | | VFIFPPSDEQLKS | | FTISRDN |
| | | | DKTHTCPPCPAPELLGGPS | | FDIWGQGTL | | GTASVVCLLNN | | SKNTLY |
| | | | VFLFPPKPKDTLMISRTPE | | VTVSS | | FYPREAKVQWK | | LQMNSL |
| | | | VTCVVVDVSHEDPEVKFN | | | | VDNALQSGNSQ | | RAEDTA |
| | | | WYVDGVEVHNAKTKPRE | | | | ESVTEQDSKDST | | VYYCAR |
| | | | EQYNSTYRVVSVLTVLHQ | | | | YSLSSTLTLSKA | | WGDEGF |
| | | | DWLNGKEYKCKVSNKAL | | | | DYEKHKVYACE | | DIWGQG |
| | | | PAPIEKTISKAKGQPREPQ | | | | VTHQGLSSPVTK | | TLVTVS |
| | | | VYTLPPSRDELTKNQVSL | | | | SFNRGEC | | S |
| | | | WCLVKGFYPSDIAVEWES | | | | | | |
| | | | NGQPENNYKTTPPVLDSD | | | | | | |
| | | | GSFFLYSKLTVDKSRWQQ | | | | | | |
| | | | GNVFSCSVMHEALHNHYT | | | | | | |
| | | | QKSLSLSPGK | | | | | | |
| EPI1784 | RNF43 | 684 | QIQLVQSGPELKKPGETVK | 685 | QIQLVQSGP | 686 | QAVVTQESALT | 687 | QAVVTQE |
| (SC37. | | | ISCKASGYTFTNYGMNWV | | ELKKPGETV | | TSPGETVTFTCR | | SALTTSPG |
| 3) | | | RQAPGKGLKWVGWINTN | | KISCKASGY | | SSTGAVTTSNY | | ETVTFTC |
| | | | TGEPTYADDFKGRFAFSLE | | TFTNYGMNW | | ANWVQEKPDHL | | RSSTGAV |
| | | | TSASTAYLQISNLKNEDMS | | VRQAPGKGL | | FTGLIGGTNNRA | | TTSNYAN |
| | | | TYFCAGSHDYSFAYWGQ | | KWVGWINTN | | PGVPARFSGSLI | | WVQEKPD |
| | | | GTLVTVSAASTKGPSVFPL | | TGEPTYADD | | GDKAALTITGA | | HLFTGLIG |
| | | | APSSKSTSGGTAALGCLV | | FKGRFAFSL | | QTEDEAIYFCAL | | GTNNRAP |
| | | | KDYFPEPVTVSWNSGALT | | ETSASTAYL | | WYSNHWVFGG | | GVPARFS |
| | | | SGVHTFPAVLQSSGLYSLS | | QISNLKNED | | GTKLTVLRTVA | | GSLIGDK |
| | | | SVVTVPSSSLGTQTYICNV | | MSTYFCAGS | | APSVFIFPPSDEQ | | AALTITG |
| | | | NHKPSNTKVDKKVEPKSC | | HDYSFAYWG | | LKSGTASVVCL | | AQTEDEA |
| | | | DKTHTCPPCPAPELLGGPS | | QGTLVTVSA | | LNNFYPREAKV | | IYFCALW |
| | | | VFLFPPKPKDTLMISRTPE | | | | QWKVDNALQS | | YSNHWVF |
| | | | VTCVVVDVSHEDPEVKFN | | | | GNSQESVTEQD | | GGGTKLT |
| | | | WYVDGVEVHNAKTKPRE | | | | SKDSTYSLSSTL | | VL |
| | | | EQYNSTYRVVSVLTVLHQ | | | | TLSKADYEKHK | | |
| | | | DWLNGKEYKCKVSNKAL | | | | VYACEVTHQGL | | |
| | | | PAPIEKTISKAKGQPREPQ | | | | SSPVTKSFNRGE | | |
| | | | VYTLPPSRDELTKNQVSLT | | | | C | | |
| | | | CLVKGFYPSDIAVEWESN | | | | | | |
| | | | GQPENNYKTTPPVLDSDG | | | | | | |
| | | | SFFLYSKLTVDKSRWQQG | | | | | | |
| | | | NVFSCSVMHEALHNHYTQ | | | | | | |
| | | | KSLSLSPGK | | | | | | |
| EPI1785 | RNF43 | 688 | QVQLQQSGTELMKPGASV | 689 | QVQLQQSGT | 690 | DIQMTQSSSSFS | 691 | DIQMTQS |
| (SC37. | | | KISCKATGYTFSSYWIEW | | ELMKPGASV | | VSLGDRVTITCK | | SSSFSVSL |
| 23) | | | VKQRPGHGLEWIGEILPGS | | KISCKATGY | | ASEDIYNRLAW | | GDRVTIT |
| | | | GSTNYNEKFKGKATFTVD | | TFSSYWIEW | | FQQKPGNAPRL | | CKASEDI |
| | | | TSSNTAYMQLSSLTSEDSA | | VKQRPGHGL | | LISGATSLETGV | | YNRLAWF |
| | | | VYYCARIIRDFWGQGTTL | | EWIGEILPG | | PSRFSGSRSGED | | QQKPGNA |
| | | | TVSSASTKGPSVFPLAPSS | | SGSTNYNEK | | YTLIITSLQTEDV | | PRLLISGA |
| | | | KSTSGGTAALGCLVKDYF | | FKGKATFTV | | ATYYCQQFWTT | | TSLETGVP |
| | | | PEPVTVSWNSGALTSGVH | | DTSSNTAYM | | PPTFGGGTKLEI | | SRFSGSRS |
| | | | TFPAVLQSSGLYSLSSVVT | | QLSSLTSED | | KRTVAAPSVFIF | | GEDYTLII |
| | | | VPSSSLGTQTYICNVNHKP | | SAVYYCARI | | PPSDEQLKSGTA | | TSLQTED |
| | | | SNTKVDKKVEPKSCDKTH | | IRDFWGQGT | | SVVCLLNNFYP | | VATYYCQ |
| | | | TCPPCPAPELLGGPSVFLF | | TLTVSS | | REAKVQWKVD | | QFWTTPP |
| | | | PPKPKDTLMISRTPEVTCV | | | | NALQSGNSQES | | TFGGGTK |
| | | | VVDVSHEDPEVKFNWYVD | | | | VTEQDSKDSTY | | LEIK |
| | | | GVEVHNAKTKPREEQYNS | | | | SLSSTLTLSKAD | | |
| | | | TYRVVSVLTVLHQDWLN | | | | YEKHKVYACEV | | |
| | | | GKEYKCKVSNKALPAPIE | | | | THQGLSSPVTKS | | |
| | | | KTISKAKGQPREPQVYTLP | | | | FNRGEC | | |
| | | | PSRDELTKNQVSLTCLVK | | | | | | |
| | | | GFYPSDIAVEWESNGQPE | | | | | | |
| | | | NNYKTTPPVLDSDGSFFLY | | | | | | |
| | | | SKLTVDKSRWQQGNVFSC | | | | | | |
| | | | SVMHEALHNHYTQKSLSL | | | | | | |
| | | | SPGK | | | | | | |
| EPI1789 | RNF43 | 692 | QVQLQQSGAELMKPGAS | 693 | QVQLQQSGA | 694 | DIQMTQSSSSFS | 695 | DIQMTQS |
| (SC37. | | | VKISCKATGYTFSRYWIE | | ELMKPGASV | | VSLGDRVTITCK | | SSSFSVSL |
| 45) | | | WVKQRPGHGLEWIGEILP | | KISCKATGY | | ASEDIYNRLAW | | GDRVTIT |
| | | | GSGSTNYNEKFKGKATFT | | TFSRYWIEW | | YQQKPGNAPRL | | CKASEDI |
| | | | ADTSSNTAYMQLTSLTSE | | VKQRPGHGL | | LISGATSLETGV | | YNRLAW |
| | | | DSAVYFCERRGAYWGQG | | EWIGEILPG | | PSRFSGSGSGKD | | YQQKPGN |
| | | | TLVTVSAASTKGPSVFPLA | | SGSTNYNEK | | YTLSITSLQTED | | APRLLISG |
| | | | PSSKSTSGGTAALGCLVK | | FKGKATFTA | | VATYHCQQNW | | ATSLETG |
| | | | DYFPEPVTVSWNSGALTS | | DTSSNTAYM | | STPPTFGGGTKL | | VPSRFSGS |
| | | | GVHTFPAVLQSSGLYSLSS | | QLTSLTSED | | EIKRTVAAPSVF | | GSGKDYT |
| | | | VVTVPSSSLGTQTYICNVN | | AVYFSCERR | | IFPPSDEQLKSG | | LSITSLQT |
| | | | HKPSNTKVDKKVEPKSCD | | GAYWGQGTL | | TASVVCLLNNF | | EDVATYH |
| | | | KTHTCPPCPAPELLGGPSV | | VTVSA | | YPREAKVQWK | | CQQNWST |
| | | | FLFPPKPKDTLMISRTPEV | | | | VDNALQSGNSQ | | PPTFGGG |
| | | | TCVVVDVSHEDPEVKFN | | | | ESVTEQDSKDST | | TKLEIK |
| | | | WYVDGVEVHNAKTKPRE | | | | YSLSSTLTLSKA | | |
| | | | EQYNSTYRVVSVLTVLHQ | | | | DYEKHKVYACE | | |
| | | | DWLNGKEYKCKVSNKAL | | | | VTHQGLSSPVT | | |
| | | | PAPIEKTISKAKGQPREPQ | | | | KSFNRGEC | | |
| | | | VYTLPPSRDELTKNQVSLT | | | | | | |
| | | | CLVKGFYPSDIAVEWESN | | | | | | |
| | | | GQPENNYKTTPPVLDSDG | | | | | | |
| | | | SFFLYSKLTVDKSRWQQG | | | | | | |
| | | | NVFSCSVMHEALHNHYTQ | | | | | | |
| | | | KSLSLSPGK | | | | | | |
| EPI1791 | RNF43 | 696 | QVQLQQPGAELVRPGASV | 697 | QVQLQQPGA | 698 | DIQMTQSTSSLS | 699 | DIQMTQS |
| SC37.14 | | | KLSCKASGYTFTSYWMN | | ELVRPGASV | | ASLGDRVTISCR | | TSSLSASL |
| 1) | | | WVKQRPGQGLEWIGMIDP | | KLSCKASGY | | ASQDISNYLNW | | GDRVTISC |
| | | | SDSETHYSQMFKDKATLT | | TFTSYWMNW | | YQQKPDGTVKP | | RASQDISN |
| | | | VDQSSNTAYMHLSSLTSE | | VKQRPGQGL | | LIYYTSRLHSGV | | YLNWYQ |
| | | | DSAVYYCAREGYYFDGTR | | EWIGMIDPS | | PSRFSGSGSGTD | | QKPDGTV |
| | | | GIAYWGQGTLVTVSVAST | | DSETHYSQM | | YSLTISNLDQED | | KPLIYYTS |
| | | | KGPSVFPLAPSSKSTSGGT | | FKDKATLTV | | IATYFCQQGNTL | | RLHSGVP |
| | | | AALGCLVKDYFPEPVTVS | | DQSSNTAYM | | PFTFGSGTKLAI | | SRFSGSGS |
| | | | WNSGALTSGVHTFPAVLQ | | HLSSLTSED | | ERTVAAPSVFIF | | GTDYSLTI |
| | | | SSGLYSLSSVVTVPSSSLG | | SAVYYCARE | | PPSDEQLKSGTA | | SNLDQEDI |
| | | | TQTYICNVNHKPSNTKVD | | GYYFDGTRG | | SVVCLLNNFYP | | ATYFCQQ |
| | | | KKVEPKSCDKTHTCPPCP | | IAYWGQGTL | | REAKVQWKVD | | GNTLPFTF |
| | | | APELLGGPSVFLFPPKPKD | | VTVSV | | NALQSGNSQES | | GSGTKLAI |
| | | | TLMISRTPEVTCVVVDVS | | | | VTEQDSKDSTY | | E |
| | | | HEDPEVKFNWYVDGVEV | | | | SLSSTLTLSKAD | | |
| | | | HNAKTKPREEQYNSTYRV | | | | YEKHKVYACEV | | |
| | | | VSVLTVLHQDWLNGKEY | | | | THQGLSSPVTKS | | |
| | | | KCKVSNKALPAPIEKTISK | | | | FNRGEC | | |
| | | | AKGQPREPQVYTLPPSRD | | | | | | |
| | | | ELTKNQVSLTCLVKGFYP | | | | | | |
| | | | SDIAVEWESNGQPENNYK | | | | | | |
| | | | TTPPVLDSDGSFFLYSKLT | | | | | | |
| | | | VDKSRWQQGNVFSCSVM | | | | | | |
| | | | HEALHNHYTQKSLSLSPG | | | | | | |
| | | | K | | | | | | |
| EPI1792 | RNF43 | 700 | QVQLQQSGAEVMKPG | 701 | QVQLQQS | 702 | DIVMTQSQK | 703 | DIVMT |
| (SC37. | | | ASVKLSCKATGYTFTG | | GAEVMKP | | FMSTSVGDR | | QSQKF |
| 169) | | | YWIEWVKERPGHGLE | | GASVKLSC | | VSVTCKASQ | | MSTSV |
| | | | WIGEILPGSGSTNYNEK | | KATGYTFT | | NVGTNVAWF | | GDRVS |
| | | | FKGKATFTADPSSNTA | | GYWIEWV | | QQKPGQSPK | | VTCKA |
| | | | YMQLSSLTTEDSAIYYC | | KERPGHGL | | LLIYSASYRY | | SQNVG |
| | | | ARDYGSFGYWGQGTL | | EWIGEILP | | SGVPDRFTGS | | TNVA |
| | | | VTVSAASTKGPSVFPLA | | GSGSTNYN | | GSGTDFTLTI | | WFQQK |
| | | | PSSKSTSGGTAALGCLV | | EKFKGKAT | | SNVQSEDLA | | PGQSP |
| | | | KDYFPEPVTVSWNSGA | | FTADPSSN | | EFFCQQYNT | | KLLIYS |
| | | | LTSGVHTFPAVLQSSGL | | TAYMQLSS | | YPLTFGAGT | | ASYRY |
| | | | YSLSSVVTVPSSSLGTQ | | LTTEDSAI | | KLELKRTVA | | SGVPD |
| | | | TYICNVNHKPSNTKVD | | YYCARDY | | APSVFIFPPSD | | RFTGS |
| | | | KKVEPKSCDKTHTCPP | | GSFGYWG | | EQLKSGTAS | | GSGTD |
| | | | CPAPELLGGPSVFLFPP | | QGTLVTVS | | VVCLLNNFY | | FTLTIS |
| | | | KPKDTLMISRTPEVTCV | | A | | PREAKVQWK | | NVQSE |
| | | | VVDVSHEDPEVKFNW | | | | VDNALQSGN | | DLAEF |
| | | | YVDGVEVHNAKTKPRE | | | | SQESVTEQDS | | FCQQY |
| | | | EQYNSTYRVVSVLTVL | | | | KDSTYSLSST | | NTYPL |
| | | | HQDWLNGKEYKCKVS | | | | LTLSKADYE | | TFGAG |
| | | | NKALPAPIEKTISKAKG | | | | KHKVYACEV | | TKLEL |
| | | | QPREPQVYTLPPSRDEL | | | | THQGLSSPVT | | K |
| | | | TKNQVSLTCLVKGFYP | | | | KSFNRGEC | | |
| | | | SDIAVEWESNGQPENN | | | | | | |
| | | | YKTTPPVLDSDGSFFLY | | | | | | |
| | | | SKLTVDKSRWQQGNVF | | | | | | |
| | | | SCSVMHEALHNHYTQK | | | | | | |
| | | | SLSLSPGK | | | | | | |
| EPI1796 | RNF43 | 704 | EVQLQQSGAELVRPGA | 705 | EVQLQQSG | 706 | DIVMTQDAP | 707 | DIVMT |
| (SC37. | | | SVKLSCTASGFNIKDDY | | AELVRPGA | | SIPVTPGESVS | | QDAPSI |
| 193) | | | MHWMKQRPEQGLEWL | | SVKLSCTA | | ISCRSSKSLL | | PVTPG |
| | | | GWIDPEIGATEYASKFQ | | SGFNIKDD | | HSNGNTYLY | | ESVSIS |
| | | | GKATMTADTSSNTAYL | | YMHWMK | | WFLQRPGQS | | CRSSK |
| | | | QLSSLTSEDTAVYYCV | | QRPEQGLE | | PQLLIYRMSN | | SLLHS |
| | | | DDRRGMDYWGQGTSV | | WLGWIDP | | LASGVPDRFS | | NGNTY |
| | | | TVSSASTKGPSVFPLAP | | EIGATEYA | | GSGSGTAFTL | | LYWFL |
| | | | SSKSTSGGTAALGCLV | | SKFQGKAT | | RISRVEAEDV | | QRPGQ |
| | | | KDYFPEPVTVSWNSGA | | MTADTSSN | | GVYYCMQHL | | SPQLLI |
| | | | LTSGVHTFPAVLQSSGL | | TAYLQLSS | | EYPFTFGSGT | | YRMSN |
| | | | YSLSSVVTVPSSSLGTQ | | LTSEDTAV | | KLEIKRTVAA | | LASGV |
| | | | TYICNVNHKPSNTKVD | | YYCVDDR | | PSVFIFPPSDE | | PDRFS |
| | | | KKVEPKSCDKTHTCPP | | RGMDYWG | | QLKSGTASV | | GSGSG |
| | | | CPAPELLGGPSVFLFPP | | QGTSVTVS | | VCLLNNFYP | | TAFTL |
| | | | KPKDTLMISRTPEVTCV | | S | | REAKVQWK | | RISRVE |
| | | | VVDVSHEDPEVKFNW | | | | VDNALQSGN | | AEDVG |
| | | | YVDGVEVHNAKTKPRE | | | | SQESVTEQDS | | VYYC |
| | | | EQYNSTYRVVSVLTVL | | | | KDSTYSLSST | | MQHLE |
| | | | HQDWLNGKEYKCKVS | | | | LTLSKADYE | | YPFTF |
| | | | NKALPAPIEKTISKAKG | | | | KHKVYACEV | | GSGTK |
| | | | QPREPQVYTLPPSRDEL | | | | THQGLSSPVT | | LEIK |
| | | | TKNQVSLTCLVKGFYP | | | | KSFNRGEC | | |
| | | | SDIAVEWESNGQPENN | | | | | | |
| | | | YKTTPPVLDSDGSFFLY | | | | | | |
| | | | SKLTVDKSRWQQGNVF | | | | | | |
| | | | SCSVMHEALHNHYTQK | | | | | | |
| | | | SLSLSPGK | | | | | | |
| EPI1797 | RNF43 | 708 | QVSLKESGPGILQPSQT | 709 | QVSLKESG | 710 | DIVMSQSPSS | 711 | DIVMS |
| (SC37. | | | LSLTCSFSGFSLSTSGM | | PGILQPSQT | | LAVSVGEEV | | QSPSSL |
| 202) | | | AVGWIRQPSGRGLEWL | | LSLTCSFS | | TMSCKSSQSL | | AVSVG |
| | | | ANIWWDDSQHYNAAL | | GFSLSTSG | | LYSTNQKNY | | EEVTM |
| | | | KSRLTISKDTSKNQVFL | | MAVGWIR | | LAWYQQKPG | | SCKSS |
| | | | KIASVDTADTATYYCA | | QPSGRGLE | | QSPKLLIYWA | | QSLLY |
| | | | RSNWGRYFDYWGQGT | | WLANIWW | | STRESGVPDR | | STNQK |
| | | | TLTVSSASTKGPSVFPL | | DDSQHYN | | FTGSGSGTDF | | NYLA |
| | | | APSSKSTSGGTAALGCL | | AALKSRLT | | TLTISSVKAE | | WYQQ |
| | | | VKDYFPEPVTVSWNSG | | ISKDTSKN | | DLAVYYCQQ | | KPGQS |
| | | | ALTSGVHTFPAVLQSSG | | QVFLKIAS | | YYDYYTFGG | | PKLLIY |
| | | | LYSLSSVVTVPSSSLGT | | VDTADTA | | GTKLEIKRTV | | WASTR |
| | | | QTYICNVNHKPSNTKV | | TYYCARSN | | AAPSVFIFPPS | | ESGVP |
| | | | DKKVEPKSCDKTHTCP | | WGRYFDY | | DEQLKSGTA | | DRFTG |
| | | | PCPAPELLGGPSVFLFPP | | WGQGTTL | | SVVCLLNNF | | SGSGT |
| | | | KPKDTLMISRTPEVTCV | | TVSS | | YPREAKVQW | | DFTLTI |
| | | | VVDVSHEDPEVKFNW | | | | KVDNALQSG | | SSVKA |
| | | | YVDGVEVHNAKTKPRE | | | | NSQESVTEQ | | EDLAV |
| | | | EQYNSTYRVVSVLTVL | | | | DSKDSTYSLS | | YYCQQ |
| | | | HQDWLNGKEYKCKVS | | | | STLTLSKADY | | YYDYY |
| | | | NKALPAPIEKTISKAKG | | | | EKHKVYACE | | TFGGG |
| | | | QPREPQVYTLPPSRDEL | | | | VTHQGLSSPV | | TKLEIK |
| | | | TKNQVSLTCLVKGFYP | | | | TKSFNRGEC | | |
| | | | SDIAVEWESNGQPENN | | | | | | |
| | | | YKTTPPVLDSDGSFFLY | | | | | | |
| | | | SKLTVDKSRWQQGNVF | | | | | | |
| | | | SCSVMHEALHNHYTQK | | | | | | |
| | | | SLSLSPGK | | | | | | |
| EPI1798 | RNF43 | 712 | QVQLQQPGAELVKPGA | 713 | QVQLQQP | 714 | DIQMTQTTSS | 715 | DIQMT |
| (SC37. | | | SVKLSCKASGYTFTTY | | GAELVKPG | | LSASLGDRVT | | QTTSSL |
| 223) | | | YIYWVKQRPGQGLEWI | | ASVKLSCK | | ISCSASQGIG | | SASLG |
| | | | GGINPRNGGTNFNEKF | | ASGYTFTT | | NYLNWYQQ | | DRVTIS |
| | | | KTRATLTVDKSSSTAY | | YYIYWVK | | KPDGTVKLLI | | CSASQ |
| | | | MQLSSLTSEDSAVYYC | | QRPGQGLE | | YYTSSLNSGV | | GIGNY |
| | | | TRTFYWGQGTTLTVSS | | WIGGINPR | | PSRFSGSGSG | | LNWY |
| | | | ASTKGPSVFPLAPSSKS | | NGGTNFNE | | TDYSLTISNL | | QQKPD |
| | | | TSGGTAALGCLVKDYF | | KFKTRATL | | EPEDIATYFC | | GTVKL |
| | | | PEPVTVSWNSGALTSG | | TVDKSSST | | QQYSKLPYTF | | LIYYTS |
| | | | VHTFPAVLQSSGLYSLS | | AYMQLSSL | | GGGTKLEIKR | | SLNSG |
| | | | SVVTVPSSSLGTQTYIC | | TSEDSAVY | | TVAAPSVFIF | | VPSRFS |
| | | | NVNHKPSNTKVDKKVE | | YCTRTFY | | PPSDEQLKSG | | GSGSG |
| | | | PKSCDKTHTCPPCPAPE | | WGQGTTL | | TASVVCLLN | | TDYSL |
| | | | LLGGPSVFLFPPKPKDT | | TVSS | | NFYPREAKV | | TISNLE |
| | | | LMISRTPEVTCVVVDVS | | | | QWKVDNAL | | PEDIAT |
| | | | HEDPEVKFNWYVDGV | | | | QSGNSQESVT | | YFCQQ |
| | | | EVHNAKTKPREEQYNS | | | | EQDSKDSTYS | | YSKLP |
| | | | TYRVVSVLTVLHQDWL | | | | LSSTLTLSKA | | YTFGG |
| | | | NGKEYKCKVSNKALPA | | | | DYEKHKVYA | | GTKLEI |
| | | | PIEKTISKAKGQPREPQ | | | | CEVTHQGLSS | | K |
| | | | VYTLPPSRDELTKNQVS | | | | PVTKSFNRGE | | |
| | | | LTCLVKGFYPSDIAVE | | | | C | | |
| | | | WESNGQPENNYKTTPP | | | | | | |
| | | | VLDSDGSFFLYSKLTVD | | | | | | |
| | | | KSRWQQGNVFSCSVM | | | | | | |
| | | | HEALHNHYTQKSLSLS | | | | | | |
| | | | PGK | | | | | | |
| EPI1799 | RNF43 | 716 | EVQLQQSGAELVRPGA | 717 | EVQLQQSG | 718 | DIVMTQAAP | 719 | DIVMT |
| (SC37. | | | SVKLSCTASGFNIIVDY | | AELVRPGA | | SVPVTPGESV | | QAAPS |
| 226) | | | LHWVRQRPEQGLEWIG | | SVKLSCTA | | SISCRSSKSLL | | VPVTP |
| | | | WIDPEIGSTEYASKFQG | | SGFNIIVDY | | HSNGNTYLY | | GESVSI |
| | | | KATMTADTSSNTAYLQ | | LHWVRQR | | WFLQRPGQS | | SCRSS |
| | | | LSSLTSEDTAVYYCIID | | PEQGLEWI | | PQVLIYRMSN | | KSLLH |
| | | | GTMDYWGQGTSVTVS | | GWIDPEIG | | LASGVPDRFS | | SNGNT |
| | | | SASTKGPSVFPLAPSSK | | STEYASKF | | GSGSGTAFTL | | YLYWF |
| | | | STSGGTAALGCLVKDY | | QGKATMT | | RISRVEAEDV | | LQRPG |
| | | | FPEPVTVSWNSGALTS | | ADTSSNTA | | GVYYCMQHL | | QSPQV |
| | | | GVHTFPAVLQSSGLYSL | | YLQLSSLT | | EYPFTFGSGT | | LIYRM |
| | | | SSVVTVPSSSLGTQTYI | | SEDTAVYY | | KLEIKRTVAA | | SNLAS |
| | | | CNVNHKPSNTKVDKK | | CIIDGTMD | | PSVFIFPPSDE | | GVPDR |
| | | | VEPKSCDKTHTCPPCPA | | YWGQGTS | | QLKSGTASV | | FSGSGS |
| | | | PELLGGPSVFLFPPKPK | | VTVSS | | VCLLNNFYP | | GTAFT |
| | | | DTLMISRTPEVTCVVV | | | | REAKVQWK | | LRISRV |
| | | | DVSHEDPEVKFNWYV | | | | VDNALQSGN | | EAEDV |
| | | | DGVEVHNAKTKPREEQ | | | | SQESVTEQDS | | GVYYC |
| | | | YNSTYRVVSVLTVLHQ | | | | KDSTYSLSST | | MQHLE |
| | | | DWLNGKEYKCKVSNK | | | | LTLSKADYE | | YPFTF |
| | | | ALPAPIEKTISKAKGQP | | | | KHKVYACEV | | GSGTK |
| | | | REPQVYTLPPSRDELTK | | | | THQGLSSPVT | | LEIK |
| | | | NQVSLTCLVKGFYPSDI | | | | KSFNRGEC | | |
| | | | AVEWESNGQPENNYKT | | | | | | |
| | | | TPPVLDSDGSFFLYSKL | | | | | | |
| | | | TVDKSRWQQGNVFSCS | | | | | | |
| | | | VMHEALHNHYTQKSLS | | | | | | |
| | | | LSPGK | | | | | | |
| EPI1800 | RNF43 | 720 | EVQLQQSGAELVRPGA | 721 | EVQLQQSG | 722 | DIVMTQSQK | 723 | DIVMT |
| (SC37. | | | SVKLSCTASGFNIKDDY | | AELVRPGA | | FMSTSVGDR | | QSQKF |
| 231) | | | IHWVKQRPEQGLEWIG | | SVKLSCTA | | VSITCKASQS | | MSTSV |
| | | | WIDPENGDTKYASKFP | | SGFNIKDD | | VRPAVAWYQ | | GDRVS |
| | | | GKATMTADTSSNTAYL | | YIHWVKQ | | QKPGQSPKA | | ITCKAS |
| | | | QLSSLTSEDTAVYYCT | | RPEQGLE | | LIYLASNRHT | | QSVRP |
| | | | ASRTTALDYWGPGTTL | | WIGWIDPE | | GVPDRFTGS | | AVAW |
| | | | TVSSASTKGPSVFPLAP | | NGDTKYA | | GSGTDFTLTI | | YQQKP |
| | | | SSKSTSGGTAALGCLV | | SKFPGKAT | | SNVQSEDLA | | GQSPK |
| | | | KDYFPEPVTVSWNSGA | | MTADTSSN | | DYFCLQHWN | | ALIYL |
| | | | LTSGVHTFPAVLQSSGL | | TAYLQLSS | | YPYTFGGGT | | ASNRH |
| | | | YSLSSVVTVPSSSLGTQ | | LTSEDTAV | | KLEIKRTVAA | | TGVPD |
| | | | TYICNVNHKPSNTKVD | | YYCTASRT | | PSVFIFPPSDE | | RFTGS |
| | | | KKVEPKSCDKTHTCPP | | TALDYWG | | QLKSGTASV | | GSGTD |
| | | | CPAPELLGGPSVFLFPP | | PGTTLTVS | | VCLLNNFYP | | FTLTIS |
| | | | KPKDTLMISRTPEVTCV | | S | | REAKVQWK | | NVQSE |
| | | | VVDVSHEDPEVKFNW | | | | VDNALQSGN | | DLADY |
| | | | YVDGVEVHNAKTKPRE | | | | SQESVTEQDS | | FCLQH |
| | | | EQYNSTYRVVSVLTVL | | | | KDSTYSLSST | | WNYPY |
| | | | HQDWLNGKEYKCKVS | | | | LTLSKADYE | | TFGGG |
| | | | NKALPAPIEKTISKAKG | | | | KHKVYACEV | | TKLEIK |
| | | | QPREPQVYTLPPSRDEL | | | | THQGLSSPVT | | |
| | | | TKNQVSLTCLVKGFYP | | | | KSFNRGEC | | |
| | | | SDIAVEWESNGQPENN | | | | | | |
| | | | YKTTPPVLDSDGSFFLY | | | | | | |
| | | | SKLTVDKSRWQQGNVF | | | | | | |
| | | | SCSVMHEALHNHYTQK | | | | | | |
| | | | SLSLSPGK | | | | | | |

The sequences listed in Table 2 (SEQ ID NOs: 354-633; 684-723) are amino acid molecules. The sequences listed in Table 2 (SEQ ID NOs: 354-633; 684-723) are amino acid molecules that are synthetic constructs. The sequences listed in Table 2 (SEQ ID NOs: 354-633; 684-723) for HC sequences (heavy chain), VH sequence (variable heavy chain sequence), LC sequences (light chain), VL sequence (variable light chain sequence) are amino acid molecules that are synthetic constructs.

### Second Binding Region

In some embodiments, the second binding domain (i.e., the EGFR binding domain) comprises an EGFR binding domain derived from an anti-EGFR antibody (e.g., a CDR that specifically binds to EGFR). Such antibodies are known to those skilled in the art and can be incorporated into methods and bispecific binding agents of the present disclosure. Antibodies targeting EGFR are known in the art, and include, for example, the following anti-EGFR antibodies: (i) cetuximab, described in, for example, P. Kirkpatrick, et al., “Cetuximab.” Nature Reviews Drug Discovery, 3(7) (2004): 549; (ii) panitumumab, described in, for example, L. Saltz, et al., “Panitumamab.” Nature Reviews Drug Discovery, 5(12) (2006): 987; (iii) nimotuzumab, described in, for example, M. S. Ramakrishnan, “Nimotuzumab, a promising therapeutic monoclonal for treatment of tumors of epithelial origin.” mAbs 1(1) (2009):41; and (iv) necitumumab, described in, for example, D. R. Tabernero, “Necitumumab, a fully human IgGI mAb directed against the EGFR for the potential treatment of cancer.” Current Opinions in Investigational Drugs, 11(12) (2000): 1434.

The antibodies described in the foregoing are merely exemplary and are not meant to limit in any way the scope of the present disclosure. Additional binding agents, including antibodies, suitable for incorporation into the methods and bispecific binding agents of the present disclosure will be evident to one of ordinary skill.

In some embodiments, the second binding domain binds to a mutant EGFR protein. In some embodiments, the second binding domain selectively binds to a mutant EGFR protein.

In some embodiments, the second binding domain comprises a heavy chain (HC) sequence, a variable heavy (VH) sequence, a light chain (LC) sequence, and a variable light (VL) sequence. In some embodiments, the second binding domain comprises an HC sequence and a VH sequence. The second binding domain comprising an HC sequence, a VH sequence, an LC sequence, and a VL sequence may comprises one or more sequences listed in Table 3. The second binding domain comprising an HC sequence, a VH sequence, an LC sequence, and a VL sequence may comprise at least 70% sequence identity to one or more sequences listed in Table 3. In some cases, the second binding domain comprising an HC sequence, a VH sequence, an LC sequence, and a VL sequence comprises at least 75% sequence identity to one or more sequences listed in Table 3. In some cases, the second binding domain comprising an HC sequence, a VH sequence, an LC sequence, and a VL sequence comprises at least 80% sequence identity to one or more sequences listed in Table 3. In some cases, the second binding domain comprising an HC sequence, a VH sequence, an LC sequence, and a VL sequence comprises at least 85% sequence identity to one or more sequences listed in Table 3. In some cases, the second binding domain comprising an HC sequence, a VH sequence, an LC sequence, and a VL sequence comprises at least 90% sequence identity to one or more sequences listed in Table 3. In some cases, the second binding domain comprising an HC sequence, a VH sequence, an LC sequence, and a VL sequence comprises at least 91% sequence identity to one or more sequences listed in Table 3. In some cases, the second binding domain comprising an HC sequence, a VH sequence, an LC sequence, and a VL sequence comprises at least 92% sequence identity to one or more sequences listed in Table 3. In some cases, the second binding domain comprising an HC sequence, a VH sequence, an LC sequence, and a VL sequence comprises at least 93% sequence identity to one or more sequences listed in Table 3. In some cases, the second binding domain comprising an HC sequence, a VH sequence, an LC sequence, and a VL sequence comprises at least 94% sequence identity to one or more sequences listed in Table 3. In some cases, the second binding domain comprising an HC sequence, a VH sequence, an LC sequence, and a VL sequence comprises at least 95% sequence identity to one or more sequences listed in Table 3. In some cases, the second binding domain comprising an HC sequence, a VH sequence, an LC sequence, and a VL sequence comprises at least 96% sequence identity to one or more sequences listed in Table 3. In some cases, the second binding domain comprising an HC sequence, a VH sequence, an LC sequence, and a VL sequence comprises at least 97% sequence identity to one or more sequences listed in Table 3. In some cases, the second binding domain comprising an HC sequence, a VH sequence, an LC sequence, and a VL sequence comprises at least 98% sequence identity to one or more sequences listed in Table 3. In some cases, the second binding domain comprising an HC sequence, a VH sequence, an LC sequence, and a VL sequence comprises at least 99% sequence identity to one or more sequences listed in Table 3. In some cases, the second binding domain comprising an HC sequence, a VH sequence, an LC sequence, and a VL sequence comprises at least 99.5% sequence identity to one or more sequences listed in Table 3. In some cases, the second binding domain comprising an HC sequence, a VH sequence, an LC sequence, and a VL sequence comprises at least 99.9% sequence identity to one or more sequences listed in Table 3.

In some embodiments, the antibodies targeting EGFR comprise a sequence listed Table 3. In some embodiments, the antibodies targeting EGFR comprise a sequence listed Table 3. In some embodiments, the antibodies targeting EGFR comprise at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, at least 99.9%, or at least 99.9% sequence identity to a sequence listed Table 3.

In some embodiments, the second binding domain comprises at least 70% sequence identity to Cetuximab. In some embodiments, the second binding domain comprises at least 75% sequence identity to Cetuximab. In some embodiments, the second binding domain comprises at least 80% sequence identity to Cetuximab. In some embodiments, the second binding domain comprises at least 85% sequence identity to Cetuximab. In some embodiments, the second binding domain comprises at least 90% sequence identity to Cetuximab. In some embodiments, the second binding domain comprises at least 91% sequence identity to Cetuximab In some embodiments, the second binding domain comprises at least 92% sequence identity to Cetuximab In some embodiments, the second binding domain comprises at least 93% sequence identity to Cetuximab In some embodiments, the second binding domain comprises at least 94% sequence identity to Cetuximab. In some embodiments, the second binding domain comprises at least 95% sequence identity to Cetuximab. In some embodiments, the second binding domain comprises at least 96% sequence identity to Cetuximab. In some embodiments, the second binding domain comprises at least 97% sequence identity to Cetuximab. In some embodiments, the second binding domain comprises at least 98% sequence identity to Cetuximab. In some embodiments, the second binding domain comprises at least 99% sequence identity to Cetuximab. In some embodiments, the second binding domain comprises at least 99.5% sequence identity to Cetuximab. In some embodiments, the second binding domain comprises at least 99.9% sequence identity to Cetuximab.

In some embodiments, the second binding domain comprises at least 70% sequence identity to Mouse adenovirus 2 (Mav2). In some embodiments, the second binding domain comprises at least 75% sequence identity to Mav2. In some embodiments, the second binding domain comprises at least 80% sequence identity to Mav2. In some embodiments, the second binding domain comprises at least 85% sequence identity to Mav2. In some embodiments, the second binding domain comprises at least 90% sequence identity to Mav2. In some embodiments, the second binding domain comprises at least 91% sequence identity to Mav2 In some embodiments, the second binding domain comprises at least 92% sequence identity to Mav2 In some embodiments, the second binding domain comprises at least 93% sequence identity to Mav2 In some embodiments, the second binding domain comprises at least 94% sequence identity to Mav2. In some embodiments, the second binding domain comprises at least 95% sequence identity to Mav2. In some embodiments, the second binding domain comprises at least 96% sequence identity to Mav2. In some embodiments, the second binding domain comprises at least 97% sequence identity to Mav2. In some embodiments, the second binding domain comprises at least 98% sequence identity to Mav2. In some embodiments, the second binding domain comprises at least 99% sequence identity to Mav2. In some embodiments, the second binding domain comprises at least 99.5% sequence identity to Mav2. In some embodiments, the second binding domain comprises at least 99.9% sequence identity to Mav2. As described herein, h7D12 hIgG1 is Mav2.

In some embodiments, the second binding domain binds to an epitope of EGFR on the target cell, wherein the epitope comprises at least 70% sequence identity to an epitope to which Cetuximab binds. In some embodiments, the second binding domain binds to an epitope of EGFR on the target cell, wherein the epitope comprises at least 80% sequence identity to an epitope to which Cetuximab binds. In some cases, the second binding domain binds to an epitope of EGFR on the target cell, wherein the epitope comprises at least 90% sequence identity to the epitope to which Cetuximab binds. In some embodiments, the second binding domain binds to an epitope of EGFR on the target cell, wherein the epitope comprises at least 95% sequence identity to an epitope to which Cetuximab binds.

In some embodiments, the second binding domain binds to an epitope of EGFR on the target cell that does not include any of the amino acids from the epitope to which Cetuximab binds. In some embodiments, the second binding domain binds to an epitope of EGFR on the target cell that includes one, two, three, four, five, or six of the amino acids from the epitope to which Cetuximab binds. In some embodiments, the second binding domain binds to an epitope of EGFR on the target cell that includes one or more of the amino acids from the epitope to which Cetuximab binds. In some embodiments, the second binding domain binds to an epitope of EGFR on the target cell that includes two or more of the amino acids from the epitope to which Cetuximab binds. In some embodiments, the second binding domain binds to an epitope of EGFR on the target cell that includes three or more of the amino acids from the epitope to which Cetuximab binds. In some embodiments, the second binding domain binds to an epitope of EGFR on the target cell that includes four or more of the amino acids from the epitope to which Cetuximab binds.

In some embodiments, the second binding domain binds to EGFR on the target cell, wherein the epitope comprises at least 70% sequence identity to an epitope to which Mav2 binds. In some embodiments, the second binding domain binds to an epitope of EGFR on the target cell, wherein the epitope comprises at least 80% sequence identity to an epitope to which Mav2 binds. In some cases, the second binding domain binds to an epitope of EGFR on the target cell, wherein the epitope comprises at least 90% sequence identity to the epitope to which Mav2 binds. In some embodiments, the second binding domain binds to an epitope of EGFR on the target cell, wherein the epitope comprises at least 95% sequence identity to an epitope to which Mav2 binds.

In some embodiments, the second binding domain binds to an epitope of EGFR on the target cell that does not include any of the amino acids from the epitope to which Mav2 binds. In some embodiments, the second binding domain binds to an epitope of EGFR on the target cell that includes one, two, three, four, five, or six of the amino acids from the epitope to which Mav2 binds. In some embodiments, the second binding domain binds to an epitope of EGFR on the target cell that includes one or more of the amino acids from the epitope to which Mav2 binds. In some embodiments, the second binding domain binds to an epitope of EGFR on the target cell that includes two or more of the amino acids from the epitope to which Mav2 binds. In some embodiments, the second binding domain binds to an epitope of EGFR on the target cell that includes three or more of the amino acids from the epitope to which Mav2 binds. In some embodiments, the second binding domain binds to an epitope of EGFR on the target cell that includes four or more of the amino acids from the epitope to which Mav2 binds.

In some embodiments, the epitope of EGFR comprises the following amino acids of human EGFR (UniProt ID: P00533): P373, R377, L406, Q407, Q432, H433, Q435, F436, V441, S442, 1462, S464, G465, K467, K489, I490, I491, S492, N493, G495, and N497. The antibodies targeting EGFR may target the epitope comprising the amino acids P373, R377, L406, Q407, Q432, H433, Q435, F436, V441, S442, 1462, S464, G465, K467, K489, 1490, 1491, S492, N493, G495, and N497 of human EGFR. In some embodiments, the antibody targeting the amino acids P373, R377, L406, Q407, Q432, H433, Q435, F436, V441, S442, 1462, S464, G465, K467, K489, I490, I491, S492, N493, G495, and N497 of human EGFR comprises Cetuximab. In some embodiments, the epitope of EGFR comprises the following amino acids of human EGFR: L349, H370, L372, P373, V374, R377, D379, F381, T382, Q408, H433, S442. The antibodies targeting EGFR may target the epitope comprising the amino acids L349, H370, L372, P373, V374, R377, D379, F381, T382, Q408, H433, and S442 of human EGFR. In some embodiments, the antibody targeting the amino acids L349, H370, L372, P373, V374, R377, D379, F381, T382, Q408, H433, and S442 of human EGFR comprises Mav2 (h7D12 hIgG1).

In some embodiments, the antibodies targeting the internalizing receptor protein may bind the same epitope as Cetuximab binds with a similar affinity as Cetuximab. The antibodies targeting the internalizing receptor protein may bind to an epitope that comprises about 70% sequence identity to the epitope to which Cetuximab binds with a similar affinity as Cetuximab. The antibodies targeting the internalizing receptor protein may bind to an epitope that comprises about 75% sequence identity to the epitope to which Cetuximab binds with a similar affinity as Cetuximab. The antibodies targeting the internalizing receptor protein may bind to an epitope that comprises about 80% sequence identity to the epitope to which Cetuximab binds with a similar affinity as Cetuximab. The antibodies targeting the internalizing receptor protein may bind to an epitope that comprises about 85% sequence identity to the epitope to which Cetuximab binds with a similar affinity as Cetuximab. The antibodies targeting the internalizing receptor protein may bind to an epitope that comprises about 90% sequence identity to the epitope to which Cetuximab binds with a similar affinity as Cetuximab. The antibodies targeting the internalizing receptor protein may bind to an epitope that comprises about 95% sequence identity to the epitope to which Cetuximab binds with a similar affinity as Cetuximab. The antibodies targeting the internalizing receptor protein may bind to an epitope that comprises about 99% sequence identity to the epitope to which Cetuximab binds with a similar affinity as Cetuximab.

In some embodiments, the antibodies targeting the internalizing receptor protein may bind the same epitope as Cetuximab binds with a different affinity as compared to Cetuximab. The antibodies targeting the internalizing receptor protein may bind to an epitope that comprises about 70% sequence identity to the epitope to which Cetuximab binds with a different affinity as compared to Cetuximab. The antibodies targeting the internalizing receptor protein may bind to an epitope that comprises about 75% sequence identity to the epitope to which Cetuximab binds with a different affinity as compared to Cetuximab. The antibodies targeting the internalizing receptor protein may bind to an epitope that comprises about 80% sequence identity to the epitope to which Cetuximab binds with a different affinity as compared to Cetuximab. The antibodies targeting the internalizing receptor protein may bind to an epitope that comprises about 85% sequence identity to the epitope to which Cetuximab binds with a different affinity as compared to Cetuximab. The antibodies targeting the internalizing receptor protein may bind to an epitope that comprises about 90% sequence identity to the epitope to which Cetuximab binds with a different affinity as compared to Cetuximab. The antibodies targeting the internalizing receptor protein may bind to an epitope that comprises about 95% sequence identity to the epitope to which Cetuximab binds with a different affinity as compared to Cetuximab. The antibodies targeting the internalizing receptor protein may bind to an epitope that comprises about 99% sequence identity to the epitope to which Cetuximab binds with a different affinity as compared to Cetuximab.

In some embodiments, the antibodies targeting the internalizing receptor protein may bind the same epitope as Mav2 binds with a similar affinity as Mav2. The antibodies targeting the internalizing receptor protein may bind to an epitope that comprises about 70% sequence identity to the epitope to which Mav2 binds with a similar affinity as Mav2. The antibodies targeting the internalizing receptor protein may bind to an epitope that comprises about 75% sequence identity to the epitope to which Mav2 binds with a similar affinity as Mav2. The antibodies targeting the internalizing receptor protein may bind to an epitope that comprises about 80% sequence identity to the epitope to which Mav2 binds with a similar affinity as Mav2. The antibodies targeting the internalizing receptor protein may bind to an epitope that comprises about 85% sequence identity to the epitope to which Mav2 binds with a similar affinity as Mav2. The antibodies targeting the internalizing receptor protein may bind to an epitope that comprises about 90% sequence identity to the epitope to which Mav2 binds with a similar affinity as Mav2. The antibodies targeting the internalizing receptor protein may bind to an epitope that comprises about 95% sequence identity to the epitope to which Mav2 binds with a similar affinity as Mav2. The antibodies targeting the internalizing receptor protein may bind to an epitope that comprises about 99% sequence identity to the epitope to which Mav2 binds with a similar affinity as Mav2.

In some embodiments, the antibodies targeting the internalizing receptor protein may bind the same epitope as Mav2 binds with a different affinity as compared to Mav2. The antibodies targeting the internalizing receptor protein may bind to an epitope that comprises about 70% sequence identity to the epitope to which Mav2 binds with a different affinity as compared to Mav2. The antibodies targeting the internalizing receptor protein may bind to an epitope that comprises about 75% sequence identity to the epitope to which Mav2 binds with a different affinity as compared to Mav2. The antibodies targeting the internalizing receptor protein may bind to an epitope that comprises about 80% sequence identity to the epitope to which Mav2 binds with a different affinity as compared to Mav2. The antibodies targeting the internalizing receptor protein may bind to an epitope that comprises about 85% sequence identity to the epitope to which Mav2 binds with a different affinity as compared to Mav2. The antibodies targeting the internalizing receptor protein may bind to an epitope that comprises about 90% sequence identity to the epitope to which Mav2 binds with a different affinity as compared to Mav2. The antibodies targeting the internalizing receptor protein may bind to an epitope that comprises about 95% sequence identity to the epitope to which Mav2 binds with a different affinity as compared to Mav2. The antibodies targeting the internalizing receptor protein may bind to an epitope that comprises about 99% sequence identity to the epitope to which Mav2 binds with a different affinity as compared to Mav2.

In some cases, the antibodies targeting EGFR may bind the same epitope as Cetuximab or Mav2 (h7D12 hIgG1). The antibodies targeting EGFR may bind to an epitope that comprises about 70% sequence identity to the epitope to which Cetuximab or Mav2 (h7D12 hIgG1) binds. The antibodies targeting EGFR may bind to an epitope that comprises about 75% sequence identity to the epitope to which Cetuximab or Mav2 (h7D12 hIgG1) binds. The antibodies targeting EGFR may bind to an epitope that comprises about 80% sequence identity to the epitope to which Cetuximab or Mav2 (h7D12 hIgG1) binds. The antibodies targeting EGFR may bind to an epitope that comprises about 85% sequence identity to the epitope to which Cetuximab or Mav2 (h7D12 hIgG1) binds. The antibodies targeting EGFR may bind to an epitope that comprises about 90% sequence identity to the epitope to which Cetuximab or Mav2 (h7D12 hIgG1) binds. The antibodies targeting EGFR may bind to an epitope that comprises about 95% sequence identity to the epitope to which Cetuximab or Mav2 (h7D12 hIgG1) binds. The antibodies targeting EGFR may bind to an epitope that comprises about 99% sequence identity to the epitope to which Cetuximab or Mav2 (h7D12 hIgG1) binds.

The antibodies targeting EGFR may bind to an epitope that comprises a different epitope than the epitope to which Cetuximab or Mav2 (h7D12 hIgG1) binds. The antibodies targeting EGFR may bind to an epitope that comprises a different epitope than the epitope to which Cetuximab or Mav2 (h7D12 hIgG1) binds, wherein the epitopes do not bind to any of the same amino acids on EGFR. The antibodies targeting EGFR may bind to an epitope that comprises a different epitope than the epitope to which Cetuximab or Mav2 (h7D12 hIgG1) binds, wherein the epitopes bind to any one or more of the same amino acids on EGFR. The antibodies targeting EGFR may bind to an epitope that comprises a different epitope than the epitope to which Cetuximab or Mav2 (h7D12 hIgG1) binds, wherein the epitopes bind to any two or more of the same amino acids on EGFR. The antibodies targeting EGFR may bind to an epitope that comprises a different epitope than the epitope to which Cetuximab or Mav2 (h7D12 hIgG1) binds, wherein the epitopes bind to any three or more of the same amino acids on EGFR. The antibodies targeting EGFR may bind to an epitope that comprises a different epitope than the epitope to which Cetuximab or Mav2 (h7D12 hIgG1) binds, wherein the epitopes bind to any four or more of the same amino acids on EGFR. The antibodies targeting EGFR may bind to an epitope that comprises a different epitope than the epitope to which Cetuximab or Mav2 (h7D12 hIgG1) binds, wherein the epitopes bind to any five or more of the same amino acids on EGFR. The antibodies targeting EGFR may bind to an epitope that comprises a different epitope than the epitope to which Cetuximab or Mav2 (h7D12 hIgG1) binds, wherein the epitopes bind to any six or more of the same amino acids on EGFR. The antibodies targeting EGFR may bind to an epitope that comprises a different epitope than the epitope to which Cetuximab or Mav2 (h7D12 hIgG1) binds, wherein the epitopes bind to any seven or more of the same amino acids on EGFR. The antibodies targeting EGFR may bind to an epitope that comprises a different epitope than the epitope to which Cetuximab or Mav2 (h7D12 hIgG1) binds, wherein the epitopes bind to any eight or more of the same amino acids on EGFR. The antibodies targeting EGFR may bind to an epitope that comprises a different epitope than the epitope to which Cetuximab or Mav2 (h7D12 hIgG1) binds, wherein the epitopes bind to any nine or more of the same amino acids on EGFR. The antibodies targeting EGFR may bind to an epitope that comprises a different epitope than the epitope to which Cetuximab or Mav2 (h7D12 hIgG1) binds, wherein the epitopes bind to any ten or more of the same amino acids on EGFR.

In some cases, the antibodies targeting EGFR may bind the same epitope as any one of the antibodies listed in Table 3. The antibodies targeting EGFR may bind to an epitope that comprises about 70% sequence identity to the epitope to which any one of the antibodies listed in Table 3 binds. The antibodies targeting EGFR may bind to an epitope that comprises about 75% sequence identity to the epitope to which any one of the antibodies listed in Table 3 binds. The antibodies targeting EGFR may bind to an epitope that comprises about 80% sequence identity to the epitope to which any one of the antibodies listed in Table 3 binds. The antibodies targeting EGFR may bind to an epitope that comprises about 85% sequence identity to the epitope to which any one of the antibodies listed in Table 3 binds. The antibodies targeting EGFR may bind to an epitope that comprises about 90% sequence identity to the epitope to which any one of the antibodies listed in Table 3 binds. The antibodies targeting EGFR may bind to an epitope that comprises about 95% sequence identity to the epitope to which any one of the antibodies listed in Table 3 binds. The antibodies targeting EGFR may bind to an epitope that comprises about 99% sequence identity to the epitope to which any one of the antibodies listed in Table 3 binds.

In some embodiments, the antibodies targeting the internalizing receptor protein may bind the same epitope as any one of the antibodies listed in Table 3 binds with a similar affinity as any one of the antibodies listed in Table 3. The antibodies targeting the internalizing receptor protein may bind to an epitope that comprises about 70% sequence identity to the epitope to which any one of the antibodies listed in Table 3 binds with a similar affinity as any one of the antibodies listed in Table 3. The antibodies targeting the internalizing receptor protein may bind to an epitope that comprises about 75% sequence identity to the epitope to which any one of the antibodies listed in Table 3 binds with a similar affinity as any one of the antibodies listed in Table 3. The antibodies targeting the internalizing receptor protein may bind to an epitope that comprises about 80% sequence identity to the epitope to which any one of the antibodies listed in Table 3 binds with a similar affinity as any one of the antibodies listed in Table 3. The antibodies targeting the internalizing receptor protein may bind to an epitope that comprises about 85% sequence identity to the epitope to which any one of the antibodies listed in Table 3 binds with a similar affinity as any one of the antibodies listed in Table 3. The antibodies targeting the internalizing receptor protein may bind to an epitope that comprises about 90% sequence identity to the epitope to which any one of the antibodies listed in Table 3 binds with a similar affinity as any one of the antibodies listed in Table 3. The antibodies targeting the internalizing receptor protein may bind to an epitope that comprises about 95% sequence identity to the epitope to which any one of the antibodies listed in Table 3 binds with a similar affinity as any one of the antibodies listed in Table 3. The antibodies targeting the internalizing receptor protein may bind to an epitope that comprises about 99% sequence identity to the epitope to which any one of the antibodies listed in Table 3 binds with a similar affinity as any one of the antibodies listed in Table 3.

In some embodiments, the antibodies targeting the internalizing receptor protein may bind the same epitope as any one of the antibodies listed in Table 3 binds with a different affinity as compared to any one of the antibodies listed in Table 3. The antibodies targeting the internalizing receptor protein may bind to an epitope that comprises about 70% sequence identity to the epitope to which any one of the antibodies listed in Table 3 binds with a different affinity as compared to any one of the antibodies listed in Table 3. The antibodies targeting the internalizing receptor protein may bind to an epitope that comprises about 75% sequence identity to the epitope to which any one of the antibodies listed in Table 3 binds with a different affinity as compared to any one of the antibodies listed in Table 3. The antibodies targeting the internalizing receptor protein may bind to an epitope that comprises about 80% sequence identity to the epitope to which any one of the antibodies listed in Table 3 binds with a different affinity as compared to any one of the antibodies listed in Table 3. The antibodies targeting the internalizing receptor protein may bind to an epitope that comprises about 85% sequence identity to the epitope to which any one of the antibodies listed in Table 3 binds with a different affinity as compared to any one of the antibodies listed in Table 3. The antibodies targeting the internalizing receptor protein may bind to an epitope that comprises about 90% sequence identity to the epitope to which any one of the antibodies listed in Table 3 binds with a different affinity as compared to any one of the antibodies listed in Table 3. The antibodies targeting the internalizing receptor protein may bind to an epitope that comprises about 95% sequence identity to the epitope to which any one of the antibodies listed in Table 3 binds with a different affinity as compared to any one of the antibodies listed in Table 3. The antibodies targeting the internalizing receptor protein may bind to an epitope that comprises about 99% sequence identity to the epitope to which any one of the antibodies listed in Table 3 binds with a different affinity as compared to any one of the antibodies listed in Table 3.

In some embodiments, the antibodies targeting the internalizing receptor protein may bind with a similar affinity as any one of the antibodies listed in Table 3 (Table 5 lists affinities of certain monovalent binders). Table 5 describes monovalent Kds to particular internalizing receptor monovalent proteins. In certain embodiments, multispecific binding agents have a Kd less than, more than, within 10%, within 20%, within 30%, within 40%, within 50%, withing 75%, or within 100% of the binding affinity of the monovalent binding agent. For example, in Table 5, the monovalent binding affinities are described for certain CD71 monovalent binding agents. When those CD71 binding arms are incorporated in the monovalent binding agent of the disclosure, the binding affinity of the multispecific binding agent may be within an order of magnitude or an order of two-fold as the binding affinity of the monovalent binding agent. For example, the binding affinity of the monovalent binding agent has a Kd of between 0.1 nM and 100 nM. When incorporated into the multispecific binding agent, the Kd may be within the same range. Alternatively, the binding affinity may be slightly greater than, but within two fold of the monovalent binding affinity. The binding affinity may be within three fold of the monovalent binding affinity.

The antibodies targeting EGFR may bind to an epitope that comprises a different epitope than the epitope to which any one of the antibodies listed in Table 3 binds. The antibodies targeting EGFR may bind to an epitope that comprises a different epitope than the epitope to which any one of the antibodies listed in Table 3 binds, wherein the epitopes do not bind to any of the same amino acids on EGFR. The antibodies targeting EGFR may bind to an epitope that comprises a different epitope than the epitope to which any one of the antibodies listed in Table 3 binds, wherein the epitopes bind to any one or more of the same amino acids on EGFR. The antibodies targeting EGFR may bind to an epitope that comprises a different epitope than the epitope to which any one of the antibodies listed in Table 3 binds, wherein the epitopes bind to any two or more of the same amino acids on EGFR. The antibodies targeting EGFR may bind to an epitope that comprises a different epitope than the epitope to which any one of the antibodies listed in Table 3 binds, wherein the epitopes bind to any three or more of the same amino acids on EGFR. The antibodies targeting EGFR may bind to an epitope that comprises a different epitope than the epitope to which any one of the antibodies listed in Table 3 binds, wherein the epitopes bind to any four or more of the same amino acids on EGFR. The antibodies targeting EGFR may bind to an epitope that comprises a different epitope than the epitope to which any one of the antibodies listed in Table 3 binds, wherein the epitopes bind to any five or more of the same amino acids on EGFR. The antibodies targeting EGFR may bind to an epitope that comprises a different epitope than the epitope to which any one of the antibodies listed in Table 3 binds, wherein the epitopes bind to any six or more of the same amino acids on EGFR. The antibodies targeting EGFR may bind to an epitope that comprises a different epitope than the epitope to which any one of the antibodies listed in Table 3 binds, wherein the epitopes bind to any seven or more of the same amino acids on EGFR. The antibodies targeting EGFR may bind to an epitope that comprises a different epitope than the epitope to which any one of the antibodies listed in Table 3 binds, wherein the epitopes bind to any eight or more of the same amino acids on EGFR. The antibodies targeting EGFR may bind to an epitope that comprises a different epitope than the epitope to which any one of the antibodies listed in Table 3 binds, wherein the epitopes bind to any nine or more of the same amino acids on EGFR. The antibodies targeting EGFR may bind to an epitope that comprises a different epitope than the epitope to which any one of the antibodies listed in Table 3 binds, wherein the epitopes bind to any ten or more of the same amino acids on EGFR.

TABLE 3
Exemplary antibody sequences targeting EGFR.

| | SEQ | | | VH | SEQ ID | | SEQ ID | VL |
|---|---|---|---|---|---|---|---|---|
| Target | ID NO | HC sequence | SEQ ID NO | sequence | NO | LC sequence | NO | sequence |
| EGFR | 634 | QVQLVESGGGVVQPGR | 635 | QVQLVESG | 636 | AIQLTQSPS | 637 | AIQLTQSP |
| (AA11035) | | SLRLSCAASGFTFSTYG | | GGVVQPGRS | | SLSASVGDR | | SSLSASVG |
| | | MHWVRQAPGKGLEWV | | LRLSCAASG | | VTITCRASQ | | DRVTITCR |
| | | AVIWDDGSYKYYGDSV | | FTFSTYGMH | | DISSALVWY | | ASQDISSAL |
| | | KGRFTISRDNSKNTLYL | | WVRQAPGK | | QQKPGKAPK | | VWYQQKP |
| | | QMNSLRAEDTAVYYCA | | GLEWVAVI | | LLIYDASSLE | | GKAPKLLI |
| | | RDGITMVRGVMKDYFD | | WDDGSYKY | | SGVPSRFSGS | | YDASSLES |
| | | YWGQGTLVTVSSAKTT | | YGDSVKGRF | | ESGTDFTLTI | | GVPSRFSG |
| | | PPSVYPLAPGSAAQTNS | | TISRDNSKN | | SSLQPEDFA | | SESGTDFT |
| | | MVTLGCLVKGYFPEPV | | TLYLQMNSL | | TYYCQQFNS | | LTISSLQPE |
| | | TVTWNSGSLSSGVHTFP | | RAEDTAVY | | YPLTFGGGT | | DFATYYCQ |
| | | AVLQSDLYTLSSSVTVP | | YCARDGITM | | KVEIKRADA | | QFNSYPLT |
| | | SSPRPSETVTCNVAHPA | | VRGVMKDY | | APTVSIFPPS | | FGGGTKVE |
| | | SSTKVDKKIVPRDCDKT | | FDYWGQGT | | SEQLTSGGA | | IK |
| | | HTCPPCPAPELLGGPSV | | LVTVSS | | SVVCFLNNF | | |
| | | FLFPPKPKDTLMISRTPE | | | | YPKDINVKW | | |
| | | VTCVVVDVSHEDPEVK | | | | KIDGSERQN | | |
| | | FNWYVDGVEVHNAKT | | | | GVLNSWTD | | |
| | | KPREEQYNSTYRVVSVL | | | | QDSKDSTYS | | |
| | | TVLHQDWLNGKEYKC | | | | MSSTLTLTK | | |
| | | KVSNKALPAPIEKTISKA | | | | DEYERHNSY | | |
| | | KGQPREPQVYTLPPSRD | | | | TCEATHKTS | | |
| | | ELTKNQVSLSCAVKGF | | | | TSPIVKSFNR | | |
| | | YPSDIAVEWESNGQPEN | | | | NEC | | |
| | | NYKTTPPVLDSDGSFFL | | | | | | |
| | | VSKLTVDKSRWQQGNV | | | | | | |
| | | FSCSVMHEALHNHYTQ | | | | | | |
| | | KSLSLSPGK | | | | | | |
| EGFR | 638 | QVQLVQSGSELKKPGA | 639 | QVQLVQSG | 640 | DIQMTQSP | 641 | DIQMTQS |
| (AA1212) | | SVKISCKASGYDFTNYA | | SELKKPGAS | | SSLSASVGD | | PSSLSASV |
| | | MNWVRQAPGHGLEWM | | VKISCKASG | | RVTITCRAS | | GDRVTITC |
| | | GWINANTGDPTYAQGF | | YDFTNYAM | | QSISSYLNW | | RASQSISSY |
| | | TGRFVFSLDTSVSTAYL | | NWVRQAPG | | YQQKPGKAP | | LNWYQQK |
| | | QISSLKAEDSAVYYCTR | | HGLEWMGW | | KLLIYAASSL | | PGKAPKLL |
| | | ERFLEWLHFDYWGQGT | | INANTGDPT | | QSGVPSRFS | | IYAASSLQS |
| | | LVTVSSAKTTPPSVYPL | | YAQGFTGRF | | GSGSGTDFT | | GVPSRFSG |
| | | APGSAAQTNSMVTLGC | | VFSLDTSVS | | LTISSLQPED | | SGSGTDFT |
| | | LVKGYFPEPVTVTWNS | | TAYLQISSLK | | FATYYCQQS | | LTISSLQPE |
| | | GSLSSGVHTFPAVLQSD | | AEDSAVYYC | | YSTPPTFGQ | | DFATYYCQ |
| | | LYTLSSSVTVPSSPRPSE | | TRERFLEWL | | GTKVEIKRA | | QSYSTPPTF |
| | | TVTCNVAHPASSTKVD | | HFDYWGQG | | DAAPTVSIFP | | GQGTKVEI |
| | | KKIVPRDCDKTHTCPPC | | TLVTVSS | | PSSEQLTSG | | K |
| | | PAPELLGGPSVFLFPPKP | | | | GASVVCFLN | | |
| | | KDTLMISRTPEVTCVVV | | | | NFYPKDINV | | |
| | | DVSHEDPEVKFNWYVD | | | | KWKIDGSER | | |
| | | GVEVHNAKTKPREEQY | | | | QNGVLNSW | | |
| | | NSTYRVVSVLTVLHQD | | | | TDQDSKDST | | |
| | | WLNGKEYKCKVSNKAL | | | | YSMSSTLTL | | |
| | | PAPIEKTISKAKGQPREP | | | | TKDEYERHN | | |
| | | QVYTLPPSRDELTKNQV | | | | SYTCEATHK | | |
| | | SLSCAVKGFYPSDIAVE | | | | TSTSPIVKSF | | |
| | | WESNGQPENNYKTTPP | | | | NRNEC | | |
| | | VLDSDGSFFLVSKLTVD | | | | | | |
| | | KSRWQQGNVFSCSVMH | | | | | | |
| | | EALHNHYTQKSLSLSPG | | | | | | |
| | | K | | | | | | |
| EGFR | 642 | QVQLQESGGGSVQAGG | 643 | QVQLQESG | | | | |
| (AA1593) | | SLKLSCAASGRSFSTYA | | GGSVQAGGS | | | | |
| | | MGWFRQAPGQDREFVA | | LKLSCAASG | | | | |
| | | TISWTDSTDYADSVKGR | | RSFSTYAMG | | | | |
| | | FTISRDNAKNTGYLQM | | WFRQAPGQ | | | | |
| | | NSLKPEDTAVYYCAAD | | DREFVATIS | | | | |
| | | RWASSRRNVDYDYWG | | WTDSTDYA | | | | |
| | | QGTQVTVSSGGGGSDK | | DSVKGRFTIS | | | | |
| | | THTCPPCPAPELLGGPS | | RDNAKNTG | | | | |
| | | VFLFPPKPKDTLMISRTP | | YLQMNSLKP | | | | |
| | | EVTCVVVDVSHEDPEV | | EDTAVYYC | | | | |
| | | KFNWYVDGVEVHNAK | | AADRWASS | | | | |
| | | TKPREEQYNSTYRVVSV | | RRNVDYDY | | | | |
| | | LTVLHQDWLNGKEYKC | | WGQGTQVT | | | | |
| | | KVSNKALPAPIEKTISKA | | VSS | | | | |
| | | KGQPREPQVYTLPPSRD | | | | | | |
| | | ELTKNQVSLSCAVKGF | | | | | | |
| | | YPSDIAVEWESNGQPEN | | | | | | |
| | | NYKTTPPVLDSDGSFFL | | | | | | |
| | | VSKLTVDKSRWQQGNV | | | | | | |
| | | FSCSVMHEALHNHYTQ | | | | | | |
| | | KSLSLSPGK | | | | | | |
| EGFR | 644 | QVKLVESGGGVVRPGG | 645 | QVKLVESGG | | | | |
| (AA030) | | SLTLSCAASGRTSRSYG | | GVVRPGGSL | | | | |
| | | MGWFRQAPGKEREFVS | | TLSCAASGR | | | | |
| | | GISWRGDSTGYADSVK | | TSRSYGMG | | | | |
| | | GRFTISRDNAKNSLYLQ | | WFRQAPGK | | | | |
| | | MNSLRAEDTALYYCAA | | EREFVSGIS | | | | |
| | | AAGSAWYGTLYEYDY | | WRGDSTGY | | | | |
| | | WGQGTLVTVSSEPKSC | | ADSVKGRFT | | | | |
| | | DKTHTCPPCPAPELLGG | | ISRDNAKNS | | | | |
| | | PSVFLFPPKPKDTLMISR | | LYLQMNSLR | | | | |
| | | TPEVTCVVVDVSHEDPE | | AEDTALYYC | | | | |
| | | VKFNWYVDGVEVHNA | | AAAAGSAW | | | | |
| | | KTKPREEQYNSTYRVVS | | YGTLYEYDY | | | | |
| | | VLTVLHQDWLNGKEYK | | WGQGTLVT | | | | |
| | | CKVSNKALPAPIEKTISK | | VSS | | | | |
| | | AKGQPREPQVYTLPPSR | | | | | | |
| | | DELTKNQVSLSCAVKG | | | | | | |
| | | FYPSDIAVEWESNGQPE | | | | | | |
| | | NNYKTTPPVLDSDGSFF | | | | | | |
| | | LVSKLTVDKSRWQQGN | | | | | | |
| | | VFSCSVMHEALHNHYT | | | | | | |
| | | QKSLSLSPGK | | | | | | |
| EGFR | 646 | QVQLQESGPGLVKPSET | 647 | QVQLQESG | 648 | DIQMTQSP | 649 | DIQMTQS |
| (AA1034) | | LSLTCTVSGGSVSSGDY | | PGLVKPSET | | SSLSASVGD | | PSSLSASV |
| | | YWTWIRQSPGKGLEWI | | LSLTCTVSG | | RVTITCQAS | | GDRVTITC |
| | | GHIYYSGNTNYNPSLKS | | GSVSSGDYY | | QDISNYLNW | | QASQDISN |
| | | RLTISIDTSKTQFSLKLSS | | WTWIRQSPG | | YQQKPGKAP | | YLNWYQQ |
| | | VTAADTAIYYCVRDRV | | KGLEWIGHI | | KLLIYDASN | | KPGKAPKL |
| | | TGAFDIWGQGTMVTVS | | YYSGNTNY | | LETGVPSRFS | | LIYDASNL |
| | | SAKTTPPSVYPLAPGSA | | NPSLKSRLTI | | GSGSGTDFT | | ETGVPSRF |
| | | AQTNSMVTLGCLVKGY | | SIDTSKTQFS | | FTISSLQPEDI | | SGSGSGTD |
| | | FPEPVTVTWNSGSLSSG | | LKLSSVTAA | | ATYFCQHFD | | FTFTISSLQ |
| | | VHTFPAVLQSDLYTLSS | | DTAIYYCVR | | HLPLAFGGG | | PEDIATYFC |
| | | SVTVPSSPRPSETVTCN | | DRVTGAFDI | | TKVEIKRAD | | QHFDHLPL |
| | | VAHPASSTKVDKKIVPR | | WGQGTMVT | | AAPTVSIFPP | | AFGGGTKV |
| | | DCDKTHTCPPCPAPELL | | VSS | | SSEQLTSGG | | EIK |
| | | GGPSVFLFPPKPKDTLM | | | | ASVVCFLNN | | |
| | | ISRTPEVTCVVVDVSHE | | | | FYPKDINVK | | |
| | | DPEVKFNWYVDGVEVH | | | | WKIDGSERQ | | |
| | | NAKTKPREEQYNSTYR | | | | NGVLNSWT | | |
| | | VVSVLTVLHQDWLNGK | | | | DQDSKDSTY | | |
| | | EYKCKVSNKALPAPIEK | | | | SMSSTLTLT | | |
| | | TISKAKGQPREPQVYTL | | | | KDEYERHNS | | |
| | | PPSRDELTKNQVSLSCA | | | | YTCEATHKT | | |
| | | VKGFYPSDIAVEWESNG | | | | STSPIVKSFN | | |
| | | QPENNYKTTPPVLDSDG | | | | RNEC | | |
| | | SFFLVSKLTVDKSRWQ | | | | | | |
| | | QGNVFSCSVMHEALHN | | | | | | |
| | | HYTQKSLSLSPGK | | | | | | |
| EGFR | 650 | QVKLVESGGGVVRPGG | 651 | QVKLVESG | | | | |
| (EPI123; | | SLTLSCAASGRTSRSYG | | GGVVRPGGS | | | | |
| h7D12 | | MGWFRQAPGKEREFVS | | LTLSCAASG | | | | |
| hIgG1) | | GISWRGDSTGYADSVK | | RTSRSYGMG | | | | |
| | | GRFTISRDNAKNSLYLQ | | WFRQAPGK | | | | |
| | | MNSLRAEDTALYYCAA | | EREFVSGIS | | | | |
| | | AAGSAWYGTLYEYDY | | WRGDSTGY | | | | |
| | | WGQGTLVTVSSEPKSC | | ADSVKGRFT | | | | |
| | | DKTHTCPPCPAPELLGG | | ISRDNAKNS | | | | |
| | | PSVFLFPPKPKDTLMISR | | LYLQMNSLR | | | | |
| | | TPEVTCVVVDVSHEDPE | | AEDTALYYC | | | | |
| | | VKFNWYVDGVEVHNA | | AAAAGSAW | | | | |
| | | KTKPREEQYNSTYRVVS | | YGTL YEYDY | | | | |
| | | VLTVLHQDWLNGKEYK | | WGQGTLVT | | | | |
| | | CKVSNKALPAPIEKTISK | | VSS | | | | |
| | | AKGQPREPQVYTLPPSR | | | | | | |
| | | DELTKNQVSLTCLVKGF | | | | | | |
| | | YPSDIAVEWESNGQPEN | | | | | | |
| | | NYKTTPPVLDSDGSFFL | | | | | | |
| | | YSKLTVDKSRWQQGNV | | | | | | |
| | | FSCSVMHEALHNHYTQ | | | | | | |
| | | KSLSLSPGK | | | | | | |
| EGFR | 652 | QVKLVESGGGVVRPGG | 653 | QVKLVESG | | | | |
| (EPI787; | | SLTLSCAASGRTSRSYG | | GGVVRPGGS | | | | |
| h7D12 | | MGWFRQAPGKEREFVS | | LTLSCAASG | | | | |
| hIgG1) | | GISWRGDSTGYADSVK | | RTSRSYGMG | | | | |
| | | GRFTISRDNAKNSLYLQ | | WFRQAPGK | | | | |
| | | MNSLRAEDTALYYCAA | | EREFVSGIS | | | | |
| | | AAGSAWYGTLYEYDY | | WRGDSTGY | | | | |
| | | WGQGTLVTVSSGGGGS | | ADSVKGRFT | | | | |
| | | DKTHTCPPCPAPELLGG | | ISRDNAKNS | | | | |
| | | PSVFLFPPKPKDTLMISR | | LYLQMNSLR | | | | |
| | | TPEVTCVVVDVSHEDPE | | AEDTALYYC | | | | |
| | | VKFNWYVDGVEVHNA | | AAAAGSAW | | | | |
| | | KTKPREEQYNSTYRVVS | | YGTL YEYDY | | | | |
| | | VLTVLHQDWLNGKEYK | | WGQGTLVT | | | | |
| | | CKVSNKALPAPIEKTISK | | VSS | | | | |
| | | AKGQPREPQVYTLPPSR | | | | | | |
| | | DELTKNQVSLTCLVKGF | | | | | | |
| | | YPSDIAVEWESNGQPEN | | | | | | |
| | | NYKTTPPVLDSDGSFFL | | | | | | |
| | | YSKLTVDKSRWQQGNV | | | | | | |
| | | FSCSVMHEALHNHYTQ | | | | | | |
| | | KSLSLSPGK | | | | | | |
| EGFR | 654 | QVKLVESGGGVVRPGG | 655 | QVKLVESG | | | | |
| (AA253) | | SLTLSCAASGRTSRSYG | | GGVVRPGGS | | | | |
| | | MGWFRQAPGKEREFVS | | LTLSCAASG | | | | |
| | | GISWRGDSTGYADSVK | | RTSRSYGMG | | | | |
| | | GRFTISRDNAKNSLYLQ | | WFRQAPGK | | | | |
| | | MNSLRAEDTALYYCAA | | EREFVSGIS | | | | |
| | | AAGSAWYGTLYEYDY | | WRGDSTGY | | | | |
| | | WGQGTLVTVSSGGGGS | | ADSVKGRFT | | | | |
| | | DKTHTCPPCPAPELLGG | | ISRDNAKNS | | | | |
| | | PSVFLFPPKPKDTLMISR | | LYLQMNSLR | | | | |
| | | TPEVTCVVVDVSHEDPE | | AEDTALYYC | | | | |
| | | VKFNWYVDGVEVHNA | | AAAAGSAW | | | | |
| | | KTKPREEQYNSTYRVVS | | YGTL YEYDY | | | | |
| | | VLTVLHQDWLNGKEYK | | WGQGTLVT | | | | |
| | | CKVSNKALPAPIEKTISK | | VSS | | | | |
| | | AKGQPREPQVYTLPPSR | | | | | | |
| | | DELTKNQVSLSCAVKG | | | | | | |
| | | FYPSDIAVEWESNGQPE | | | | | | |
| | | NNYKTTPPVLDSDGSFF | | | | | | |
| | | LVSKLTVDKSRWQQGN | | | | | | |
| | | VFSCSVMHEALHNHYT | | | | | | |
| | | QKSLSLSPGK | | | | | | |
| EGFR | 724 | QVKLVESGGGVVRPGG | 725 | QVKLVESG | | | | |
| (AA235) | | SLTLSCAASGRTSRSYG | | GGVVRPGGS | | | | |
| | | MGWFRQAPGKEREFVS | | LTLSCAASG | | | | |
| | | GISWRGDSTGYADSVK | | RTSRSYGMG | | | | |
| | | GRFTISRDNAKNSLYLQ | | WFRQAPGK | | | | |
| | | MNSLRAEDTALYYCAA | | EREFVSGIS | | | | |
| | | AAGSAWYGTLYEYDY | | WRGDSTGY | | | | |
| | | WGQGTLVTVSSGGGGS | | ADSVKGRFT | | | | |
| | | DKTHTCPPCPAPELLGG | | ISRDNAKNS | | | | |
| | | PSVFLFPPKPKDTLMISR | | LYLQMNSLR | | | | |
| | | TPEVTCVVVDVSHEDPE | | AEDTALYYC | | | | |
| | | VKFNWYVDGVEVHNA | | AAAAGSAW | | | | |
| | | KTKPREEQYNSTYRVVS | | YGTL YEYDY | | | | |
| | | VLTVLHQDWLNGKEYK | | WGQGTLVT | | | | |
| | | CKVSNKALPAPIEKTISK | | VSS | | | | |
| | | AKGQPREPQVYTLPPSR | | | | | | |
| | | DELTKNQVSLSCAVKG | | | | | | |
| | | FYPSDIAVEWESNGQPE | | | | | | |
| | | NNYKTTPPVLDSDGSFF | | | | | | |
| | | LVSKLTVDKSRWQQGN | | | | | | |
| | | VFSCSVMHEALHNHYT | | | | | | |
| | | QKSLSLSPGK | | | | | | |

The sequences listed in Table 3 (SEQ ID NOs: 634-655; 724-725) are amino acid molecules. The sequences listed in Table 3 (SEQ ID NOs: 634-655; 724-725) are amino acid molecules that are synthetic constructs. The sequences listed in Table 3 (SEQ ID NOs: 634-655; 724-725) for HC sequences (heavy chain), VH sequence (variable heavy chain sequence), LC sequences (light chain), VL sequence (variable light chain sequence) are amino acid molecules that are synthetic constructs.

### Synthesis

Multispecific binding agents are synthesized using the techniques of recombinant DNA and protein expression. For example, for the synthesis of DNA encoding a dual IgG of the disclosure, suitable DNA sequences encoding the constant domains of the heavy and light chains are widely available. Sequences encoding the selected variable domains are inserted by standard methods, and the resulting nucleic acids encoding full-length heavy and light chains are transduced into suitable host cells and expressed. Alternatively, the nucleic acids can be expressed in a cell-free expression system, which can provide more control over oxidation and reduction conditions, pH, folding, glycosylation, and the like.

The binding activity of the engineered antibodies of the disclosure can be assayed by any suitable method known in the art. For example, the binding activity of the engineered antibodies of the disclosure can be determined by, e.g., Scatchard analysis (Munsen et al., Analyt Biochem (1980) 107:220-39). Specific binding may be assessed using techniques known in the art including but not limited to competition ELISA, BIACORE® assays and/or KINEXA® assays. An antibody that preferentially or specifically binds (used interchangeably herein) to a target antigen or target epitope is a term well understood in the art, and methods to determine such specific or preferential binding are also known in the art. An antibody is said to exhibit specific or preferential binding if it reacts or associates more frequently, more rapidly, with greater duration and/or with greater affinity with a particular antigen or epitope than it does with alternative antigens or epitopes. An antibody specifically or preferentially binds to a target if it binds with greater affinity, avidity, more readily, and/or with greater duration than it binds to other substances. Also, an antibody specifically or preferentially binds to a target if it binds with greater affinity, avidity, more readily, and/or with greater duration to that target in a sample than it binds to other substances present in the sample. For example, an antibody that specifically or preferentially binds to a HER2 epitope is an antibody that binds this epitope with greater affinity, avidity, more readily, and/or with greater duration than it binds to other HER2 epitopes or non-HER2 epitopes. It is also understood by reading this definition, for example, that an antibody which specifically or preferentially binds to a first target antigen may or may not specifically or preferentially bind to a second target antigen. As such, specific binding and preferential binding do not necessarily require (although it can include) exclusive binding.

### Nucleic Acid Molecules

In one aspect, some embodiments disclosed herein relate to nucleic acid molecules comprising nucleotide sequences encoding the multispecific binding agents of the disclosure, including expression cassettes, and expression vectors containing these nucleic acid molecules operably linked to heterologous nucleic acid sequences such as, for example, regulatory sequences which direct in vivo expression of the protein in a host cell.

Also provided herein are vectors, plasmids, or viruses containing one or more of the nucleic acid molecules encoding any binding agent disclosed herein. The nucleic acid molecules can be contained within a vector that is capable of directing their expression in, for example, a cell that has been transformed/transduced with the vector. Suitable vectors for use in eukaryotic and prokaryotic cells are known in the art and are commercially available, or readily prepared by a skilled artisan. See for example, Sambrook, J., & Russell, D. W. (2012). Molecular Cloning: A Laboratory Manual (4th ed.). Cold Spring Harbor, NY: Cold Spring Harbor Laboratory and Sambrook, J., & Russel, D. W. (2001). Molecular Cloning: A Laboratory Manual (3rd ed.). Cold Spring Harbor, NY: Cold Spring Harbor Laboratory (jointly referred to herein as “Sambrook”); Ausubel, F. M. (1987). Current Protocols in Molecular Biology. New York, NY: Wiley (including supplements through 2014); Bollag, D. M. et al. (1996). Protein Methods. New York, NY: Wiley-Liss; Huang, L. et al. (2005). Nonviral Vectors for Gene Therapy. San Diego: Academic Press; Kaplitt, M. G. et al. (1995). Viral Vectors: Gene Therapy and Neuroscience Applications. San Diego, CA: Academic Press; Lefkovits, I. (1997). The Immunology Methods Manual: The Comprehensive Sourcebook of Techniques. San Diego, CA: Academic Press; Doyle, A. et al. (1998). Cell and Tissue Culture: Laboratory Procedures in Biotechnology. New York, NY: Wiley; Mullis, K. B., Ferré, F. & Gibbs, R. (1994). PCR: The Polymerase Chain Reaction. Boston: Birkhauser Publisher; Greenfield, E. A. (2014). Antibodies: A Laboratory Manual (2nd ed.). New York, NY: Cold Spring Harbor Laboratory Press; Beaucage, S. L. et al. (2000). Current Protocols in Nucleic Acid Chemistry. New York, NY: Wiley, (including supplements through 2014); and Makrides, S. C. (2003). Gene Transfer and Expression in Mammalian Cells. Amsterdam, NL: Elsevier Sciences B.V., the disclosures of which are incorporated herein by reference.

### Methods of Binding on Target Cancer Cells

In some embodiments, the multispecific binding agent comprising a first binding domain which specifically binds to a membrane-associated internalizing or degrading protein and a second binding domain which specifically binds to EGFR contacts a target cancer cell. In some embodiments, the multispecific binding agent comprising a first binding domain which specifically binds to a membrane-associated internalizing or degrading protein and a second binding domain which specifically binds to EGFR contacts a target bladder cancer cell. In some embodiments, the multispecific binding agent comprising a first binding domain which specifically binds to a membrane-associated internalizing or degrading protein and a second binding domain which specifically binds to EGFR contacts a target colon cancer cell. In some embodiments, the multispecific binding agent comprising a first binding domain which specifically binds to a membrane-associated internalizing or degrading protein and a second binding domain which specifically binds to EGFR contacts a target rectal cancer cell. In some embodiments, the multispecific binding agent comprising a first binding domain which specifically binds to a membrane-associated internalizing or degrading protein and a second binding domain which specifically binds to EGFR contacts a target lymphoma cancer cell. In some embodiments, the multispecific binding agent comprising a first binding domain which specifically binds to a membrane-associated internalizing or degrading protein and a second binding domain which specifically binds to EGFR contacts a target lung cancer cell. In some embodiments, the multispecific binding agent comprising a first binding domain which specifically binds to a membrane-associated internalizing or degrading protein and a second binding domain which specifically binds to EGFR contacts a target non-small cell lung cancer cell. In some embodiments, the multispecific binding agent comprising a first binding domain which specifically binds to a membrane-associated internalizing or degrading protein and a second binding domain which specifically binds to EGFR contacts a target head and neck cancer cell.

In some embodiments, the multispecific binding agent comprising a first binding domain which specifically binds to a membrane-associated internalizing or degrading protein and a second binding domain which specifically binds to EGFR contacts a target cancer cell and decreases expression of EGFR on the cancer cell by at least 40%, at least 50%, at least 55%, at least 60%, at least 65%, at least 70%, at least 75%, or at least 80%. In some embodiments, the multispecific binding agent comprising a first binding domain which specifically binds a membrane-associated internalizing or degrading protein and a second binding domain which specifically binds to EGFR contacts a target cancer cell and decreases expression of EGFR on the cancer cell by about 40%-80%, about 50%-80%, about 60%-80%, about 70%-80%, about 40%-70%, about 50%-70%, about 60%-70%, about 40%-60%, or about 50%-60%. In some embodiments, expression of EGFR on a target cell is determined relative to expression of EGFR on a control cancer cell not contacted with the binding agent.

In some embodiments, the multispecific binding agent comprising a first binding domain which specifically binds to a membrane-associated internalizing or degrading protein and a second binding domain which specifically binds to EGFR contacts a target cancer cell and increases surface removal of EGFR on a target cancer cell by at least 20%, at least 30%, at least 40%, at least 50%, at least 60%, at least 65%, at least 70%, at least 75%, at least 80%, at least 85%, or at least 90%. In some embodiments, the multispecific binding agent comprising a first binding domain which specifically binds to a membrane-associated internalizing or degrading protein and a second binding domain which specifically binds to EGFR on a target cancer cell and increases cell surface removal of EGFR by about 20-90%, about 30-90%, about 40-90%, about 50-90%, about 60-90%, about 70-90%, about 80-90%, about 20-80%, about 30-80%, about 40-80%, about 50-80%, about 60-80%, about 70-80%, about 20-70%, about 30-70%, about 40-70%, about 50-70%, about 60-70%, about 20-60%, about 30-60%, about 40-60%, about 50-60%, about 20-50%, about 30-50%, about 40-50%, about 20-40%, about 30-40%, or about 20-30%. In some embodiments, cell surface removal of EGFR on a target cell is determined relative to cell surface removal of EGFR on a control cancer cell not contacted with the binding agent. In some embodiments, the multispecific binding agent comprising a first binding domain which specifically binds to a membrane-associated internalizing or degrading protein and a second binding domain which specifically binds to EGFR contacts a target cancer cell and increases internalization of EGFR on a target cancer cell by at least 20%, at least 30%, at least 40%, at least 50%, at least 60%, at least 65%, at least 70%, at least 75%, at least 80%, at least 85%, or at least 90%. In some embodiments, the multispecific binding agent comprising a first binding domain which specifically binds to a membrane-associated internalizing or degrading protein and a second binding domain which specifically binds to EGFR on a target cancer cell and increases internalization of EGFR by about 20-90%, about 30-90%, about 40-90%, about 50-90%, about 60-90%, about 70-90%, about 80-90%, about 20-80%, about 30-80%, about 40-80%, about 50-80%, about 60-80%, about 70-80%, about 20-70%, about 30-70%, about 40-70%, about 50-70%, about 60-70%, about 20-60%, about 30-60%, about 40-60%, about 50-60%, about 20-50%, about 30-50%, about 40-50%, about 20-40%, about 30-40%, or about 20-30%. In some embodiments, internalization of EGFR on a target cell is determined relative to internalization of EGFR on a control cancer cell not contacted with the binding agent.

In some embodiments, the multispecific binding agent comprising a first binding domain which specifically binds to a membrane-associated internalizing or degrading protein and a second binding domain which specifically binds to EGFR contacts a target cancer cell and increases degradation of EGFR on a target cancer cell by at least 20%, at least 30%, at least 40%, at least 50%, at least 60%, at least 65%, at least 70%, at least 75%, at least 80%, at least 85%, or at least 90%. In some embodiments, the multispecific binding agent comprising a first binding domain which specifically binds to a membrane-associated internalizing or degrading protein and a second binding domain which specifically binds to EGFR on a target cancer cell and increases degradation of EGFR by about 20-90%, about 30-90%, about 40-90%, about 50-90%, about 60-90%, about 70-90%, about 80-90%, about 20-80%, about 30-80%, about 40-80%, about 50-80%, about 60-80%, about 70-80%, about 20-70%, about 30-70%, about 40-70%, about 50-70%, about 60-70%, about 20-60%, about 30-60%, about 40-60%, about 50-60%, about 20-50%, about 30-50%, about 40-50%, about 20-40%, about 30-40%, or about 20-30%. In some embodiments, degradation of EGFR on a target cell is determined relative to degradation of EGFR on a control cancer cell not contacted with the binding agent.

In some embodiments, the multispecific binding agent comprising a first binding domain which specifically binds to a membrane-associated internalizing or degrading protein and a second binding domain which specifically binds to EGFR contacts a target cancer cell and increases susceptibility of the cancer cell to cancer therapeutic agents. In some embodiments, the multispecific binding agent comprising a first binding domain which specifically binds to a membrane-associated internalizing or degrading protein and a second binding domain which specifically binds to EGFR contacts a target cancer cell and increases susceptibility of the cancer cell to cytotoxic agents. In some embodiments, the multispecific binding agent comprising a first binding domain which specifically binds to a membrane-associated internalizing or degrading protein and a second binding domain which specifically binds to EGFR contacts a target cancer cell and reduces proliferation of the target cancer cell. In some embodiments, the multispecific binding agent comprising a first binding domain which specifically binds to a membrane-associated internalizing or degrading protein and a second binding domain which specifically binds to EGFR contacts a target cancer cell and increases death of the cancer cell.

In some embodiments, the multispecific binding agent comprising a first binding domain which specifically binds to a membrane-associated internalizing or degrading protein and a second binding domain which specifically binds to EGFR contacts a target cancer cell in vivo.

### Pharmaceutical Compositions

In some embodiments, the multispecific binding agents, nucleic acids, and recombinant cells of the disclosure can be incorporated into compositions, including pharmaceutical compositions. Such compositions typically include the multispecific binding agents, and a pharmaceutically acceptable excipient, e.g., a carrier. Multispecific binding agents of the disclosure can be administered using formulations used for administering antibodies and antibody-based therapeutics, or formulations based thereon.

Pharmaceutical compositions suitable for injectable use include sterile aqueous solutions (where water soluble) or dispersions and sterile powders for the extemporaneous preparation of sterile injectable solutions or dispersion.

### Administration of Multispecific Binding Agents

Administration of any one or more of the therapeutic compositions described herein, e.g., multispecific binding agents and pharmaceutical compositions, can be used to treat individuals having a neoplastic disease, such as cancers.

Accordingly, in one aspect, provided herein are methods for inhibiting an activity of a target cell in an individual, the methods comprising the step of administering to the individual a first therapy including one or more of the multispecific binding agents and pharmaceutical compositions provided herein, wherein the first therapy inhibits an activity of the target cell by degrading a target surface protein. For example, an activity of the target cell may be inhibited if its proliferation is reduced, if its pathologic or pathogenic behavior is reduced, if it is destroyed or killed, or the like. Generally, the target cell of the disclosed methods can be any cancer cell.

In some embodiments, a method for treating cancer in a subject comprises administering to a subject a binding agent, wherein the binding agent comprises a first binding domain that specifically binds to a membrane-associated internalizing or degrading protein, wherein the membrane-associated internalizing or degrading protein is expressed on a target cell, and a second binding domain that specifically binds to the target protein, wherein the target protein comprises EGFR.

In some embodiments, a method for treating cancer in a subject comprises administering to a subject a binding agent, wherein the binding agent comprises a first binding domain that specifically binds to a membrane-associated internalizing or degrading protein, wherein the membrane-associated internalizing or degrading protein is expressed on a target cell, and a second binding domain that specifically binds to the target protein, wherein the target protein comprises EGFR and the method results in a decrease in EGFR expression on the target cell of at least 20%, at least 30%, at least 40%, at least 50%, at least 60%, at least 70%, at least 80%, at least 90%, at least 95%, at least 99%, or more.

In some embodiments, the method of treating cancer comprises a decrease in expression of EGFR on the target cell. In some embodiments, the method of treating cancer comprises a decrease in expression of EGFR on the target cell. In some embodiments, the method of treating cancer comprises administration of the multispecific binding agent as an individual therapeutic. In some embodiments, the method of treating cancer comprises administration of the multispecific binding agent as a combination therapeutic. In some embodiments, the combination therapeutic comprises administering the multispecific binding agent before, after, or at the same time as an additional therapeutic. In some embodiments, the additional therapeutic comprises a standard of care treatment. In some embodiments, nonlimiting examples of standard of care treatments comprise cytotoxic agents, immunotherapies, radiation, chemotherapies, surgery, hormone therapies, or a combination thereof.

In some embodiments, the method of treating cancer comprises treatment of breast cancer, B cell lymphoma, pancreatic cancer, Hodgkin's lymphoma, ovarian cancer, prostate cancer, mesothelioma, lung cancer, non-Hodgkin's B-cell (B-NHL) lymphoma, melanoma, chronic lymphocytic leukemia, acute lymphocytic leukemia, neuroblastoma, glioma, glioblastoma, bladder cancer, colorectal cancer, or head and neck cancer.

In some embodiments, a method for treating cancer in a subject comprises administering to a subject a binding agent, wherein the binding agent comprises a first binding domain that specifically binds to a membrane-associated internalizing or degrading protein, wherein the membrane-associated internalizing or degrading protein is expressed on a target cell, and a second binding domain that specifically binds to the target protein, wherein the target protein comprises EGFR and the method results in a decrease in tumor volume of at least at least 20%, at least 30%, at least 40%, at least 50%, at least 60%, at least 70%, at least 80%, at least 90%, at least 95%, or at least 99%, at least 100%, at least 125%, at least 150%, or more. In some embodiments, tumor volume of a tumor contacted with the multispecific binding agent is determined relative to the tumor volume of a tumor not contacted with the multispecific binding agent. In some embodiments, tumor volume of a tumor contacted with the multispecific binding agent is determined relative to the tumor volume of a tumor contacted with the Cetuximab.

In some embodiments, a method for treating cancer in a subject comprises administering to a subject a binding agent, wherein the binding agent comprises a first binding domain that specifically binds to a membrane-associated internalizing or degrading protein, wherein the membrane-associated internalizing or degrading protein is expressed on a target cell, and a second binding domain that specifically binds to the target protein, wherein the target protein comprises EGFR and the method results in a tumor volume of a tumor contacted with a multispecific binding agent that is less than the tumor volume of a tumor not contacted with a multispecific binding agent. In some embodiments, a method for treating cancer in a subject comprises administering to a subject a binding agent, wherein the binding agent comprises a first binding domain that specifically binds to a membrane-associated internalizing or degrading protein, wherein the membrane-associated internalizing or degrading protein is expressed on a target cell, and a second binding domain that specifically binds to the target protein, wherein the target protein comprises EGFR and the method results in a tumor volume of a tumor contacted with a multispecific binding agent that is less than the tumor volume of a tumor contacted with Cetuximab.

In some embodiments, the half-life of the multispecific binding agent is at least 20%, at least 30%, at least 40%, at least 50%, at least 60%, at least 70%, at least 80%, at least 90%, at least 100%, at least 110%, or more, as long as the half-life of Cetuximab. In some embodiments, the clearance rate of the multispecific binding agent is within at least 20%, at least 30%, at least 40%, at least 50%, at least 60%, at least 70%, at least 80%, at least 90%, at least 100%, at least 110%, or more, as compared to the clearance rate of Cetuximab.

In some embodiments, the bispecific binding agents as disclosed herein can be compared to other bispecific binding agents. In some cases, the other bispecific binding agents may target membrane associated proteins that not EGFR. In some cases, the other bispecific binding agents may target proteins that not EGFR. In some cases, the other bispecific binding agents may bind to the RSV F Protein. In some embodiments, a binding domain configured to bind to RSV F Protein comprises a sequence listed in Table 4.

TABLE 4
Exemplary binding agents targeting RSV F Protein

| Arm 2 | SEQ | | SEQ | VH | SEQ | | SEQ | VL |
|---|---|---|---|---|---|---|---|---|
| Target | ID NO | HC sequence | ID NO | sequence | ID NO | LC sequence | ID NO | sequence |
| RSV F | 656 | QVTLRESGPALVKPTQTLTL | 657 | QVTLRES | 658 | DIQMTQSPSTL | 659 | DIQMTQS |
| Protein | | TCTFSGFSLSTSGMSVGWIR | | GPALVKP | | SASVGDRVTI | | PSTLSAS |
| | | QPPGKALEWLADIWWDDK | | TQTLTLT | | TCKCQLSVGY | | VGDRVTI |
| | | KDYNPSLKSRLTISKDTSKN | | CTFSGFS | | MHWYQQKPG | | TCKCQLS |
| | | QVVLKVTNMDPADTATYY | | LSTSGMS | | KAPKLLIYDTS | | VGYMH |
| | | CARSMITNWYFDVWGAGT | | VGWIRQ | | KLASGVPSRF | | WYQQKP |
| | | TVTVSSAKTTPPSVYPLAPG | | PPGKALE | | SGSGSGTEFTL | | GKAPKL |
| | | SAAQTNSMVTLGCLVKGYF | | WLADIW | | TISSLQPDDFA | | LIYDTSK |
| | | PEPVTVTWNSGSLSSGVHTF | | WDDKKD | | TYYCFQGSGY | | LASGVPS |
| | | PAVLQSDLYTLSSSVTVPSS | | YNPSLKS | | PFTFGGGTKL | | RFSGSGS |
| | | PRPSETVTCNVAHPASSTKV | | RLTISKD | | EIKRADAAPT | | GTEFTLT |
| | | DKKIVPRDCDKTHTCPPCPA | | TSKNQV | | VSIFPPSSEQL | | ISSLQPD |
| | | PELLGGPSVFLFPPKPKDTL | | VLKVTN | | TSGGASVVCF | | DFATYY |
| | | MISRTPEVTCVVVDVSHEDP | | MDPADT | | LNNFYPKDIN | | CFQGSG |
| | | EVKFNWYVDGVEVHNAKT | | ATYYCA | | VKWKIDGSER | | YPFTFGG |
| | | KPREEQYNSTYRVVSVLTV | | RSMITN | | QNGVLNSWT | | GTKLEIK |
| | | LHQDWLNGKEYKCKVSNK | | WYFDVW | | DQDSKDSTYS | | |
| | | ALPAPIEKTISKAKGQPREP | | GAGTTV | | MSSTLTLTKD | | |
| | | QVYTLPPSRDELTKNQVSLS | | TVSS | | EYERHNSYTC | | |
| | | CAVKGFYPSDIAVEWESNG | | | | EATHKTSTSPI | | |
| | | QPENNYKTTPPVLDSDGSFF | | | | VKSFNRNEC | | |
| | | LVSKLTVDKSRWQQGNVFS | | | | | | |
| | | CSVMHEALHNHYTQKSLSL | | | | | | |
| | | SPGK | | | | | | |

The sequences listed in Table 4 (SEQ ID NOs: 656-659) are amino acid molecules. The sequences listed in Table 4 (SEQ ID NOs: 656-659) are amino acid molecules that are synthetic constructs. The sequences listed in Table 4 (SEQ ID NOs: 656-659) for HC sequences (heavy chain), VH sequence (variable heavy chain sequence), LC sequences (light chain), VL sequence (variable light chain sequence) are amino acid molecules that are synthetic constructs.

### EXAMPLES

The following examples are illustrative and non-limiting to the scope of the compositions, devices, and methods disclosed herein.

### Cell Lines:

Cells are grown in complete growth medium and maintained at 37° C. and 5% CO₂

### Example 1—Bispecific Antibody Expression

Bispecifics are expressed and purified from mammalian cells (exemplary: Expi293F, ExpiCHO-S) using transient transfection following the manufacturer's protocol. At designated time point (exemplary: 4-14 days), media is harvested by centrifugation at 4,000×g for 20 min. Tagged bispecifics and knob half IgGs are purified by Ni-NTA or Protein A affinity chromatography and buffer exchanged into PBS containing 20% glycerol, concentrated, and flash frozen for storage at −80° C. IgGs and hole half IgGs are purified by Protein A affinity chromatography and buffer exchanged into PBS containing 20% glycerol. Knob and hole half IgGs are recombined under reducing conditions (exemplary: 10 mM Tris pH 7.5, 100 mM NaCl, 20% 800 mM L-Arg pH 10 plus 200 fold excess reduced glutathione), and then purified by Ni-NTA affinity chromatography, buffer exchanged into PBS containing 20% glycerol, concentrated, and flash frozen for storage at −80° C. Purity and integrity of all proteins are assessed by SDS-PAGE and SEC

### Example 2—Stable Cell Line Generation

N-terminally epitope tagged (exemplary: alfa, HA, Myc, etc.) receptors (e.g., EGFR) are cloned into a pLVX lentiviral vector. Lentivirus is produced by transfecting HEK293T cells with standard packaging vectors. Stable cell lines expressing epitope tagged receptors are selected with puromycin and validated for expression by flow cytometry using anti-epitope tag primary antibody.

### Example 3—Degradation Experiments

Cells (exemplary examples: human cancer cell lines, primary human immune cells, or stable cell lines generated herein) are plated (exemplary examples: in 6, 12, 24, 48, 96, or 348-well plates) and grown to ˜70% confluency before treatment. Media is aspirated and cells are treated with (concentration range: 0.001 to 1000 nM; time range: 0-7 days) bispecifics (including, for example, any antibody disclosed herein) or control antibodies in complete growth medium. After incubation at 37° C., cells are washed with phosphate-buffered saline (PBS). Samples are then tested following western blotting, in-cell western blotting, or flow cytometry protocols to quantify target protein levels.

### Example 4—EGFR Level Quantification by Western Blotting

Cells are lifted with versene and harvested by centrifugation at 300×g for 5 min at 4° C. Cell pellets are lysed with 1×RIPA buffer containing cOmplete mini protease inhibitor cocktail (Sigma-Aldrich) at 4° C. for 30 min. Lysates are centrifuged at 2,000 (for 96-well plate) or 16,000 xg for 10 min at 4° C. 4× NuPAGE LDS sample buffer (Invitrogen) and 2-mercaptoethanol (BME) is added to the lysates and boiled for 10 min. Equal amounts of lysates is loaded onto a 4-12% Bis-Tris gel and ran at 200V for 37 min. The gel is incubated in 20% ethanol for 10 min and transferred onto a polyvinylidene difluoride (PVDF) membrane. The membrane is blocked in PBS with 0.1% Tween-20+5% bovine serum albumin (BSA) for 30 min at room temperature with gentle shaking. Membranes are incubated for 1 hr with primary antibodies at respective dilutions at room temp with gentle shaking in PBS+0.2% Tween-20+5% BSA. Membranes are washed four times with tris-buffered saline (TBS)+0.1% Tween-20 and then co-incubated with secondary antibodies in PBS+0.2% Tween-20+5% BSA for 1 hr at room temperature. Membranes are washed four times with TBS+0.1% Tween-20, then washed with PBS. Membranes are imaged using an Odyssey CLx Imager (LI-COR). Band intensities are quantified using Image Studio software (LI-COR).

### Example 5—EGFR Level Quantification by in-Cell Western Blotting

Fixation solution (exemplary: 4% paraformaldehyde in PBS) is added to cells and incubated for 20 min at room temperature without agitation. The fixation solution is then removed, and cells washed with PBS. Permeabilization solution (exemplary: 0.1% Triton-X100 in PBS) is added to cells and incubated for 20 min with shaking. Permeabilization solution is removed and cells are incubated in blocking buffer for 1 hr at room temperature with shaking. Blocking buffer is removed and cells are incubated with primary antibodies for 2 hr with shaking. Cells are washed four times with TBS+0.1% Tween-20. Cells are then incubated with secondary antibodies for 1 hr with shaking. Cells are then washed four times with TBS+0.1% Tween-20. Wash solution is removed and plates are imaged using an Odyssey CLx Imager (LI-COR). Well intensities are quantified using Empiria Studo software (LI-COR).

### Example 6—EGFR Level Quantification by Flow Cytometry

Cells are lifted with versene and harvested by centrifugation at 300×g for 5 min at 4° C. Cell pellets are washed with cold PBS and centrifuged at 300×g for 5 min. Cells are blocked with cold PBS+3% BSA and centrifuged (300 xg for 5 min). Cells are incubated with primary antibodies diluted in PBS+3% BSA for 30 min at 4° C. Cells are washed three times with cold PBS+3% BSA and secondary antibodies (if applicable) diluted in PBS+3% BSA added and incubated for 30 min at 4° C. Cells are washed three times with cold PBS+3% BSA and resuspended in cold PBS. Flow cytometry is performed on a CytoFLEX cytometer (Beckman Coulter) and gating is performed on single cells and live cells before acquisition of 10,000 cells. Analysis is performed using the FlowJo software package.

### Example 7—Cell Surface Removal of EGFR Using Bispecifics that Bind to EGFR and a Degrader Protein

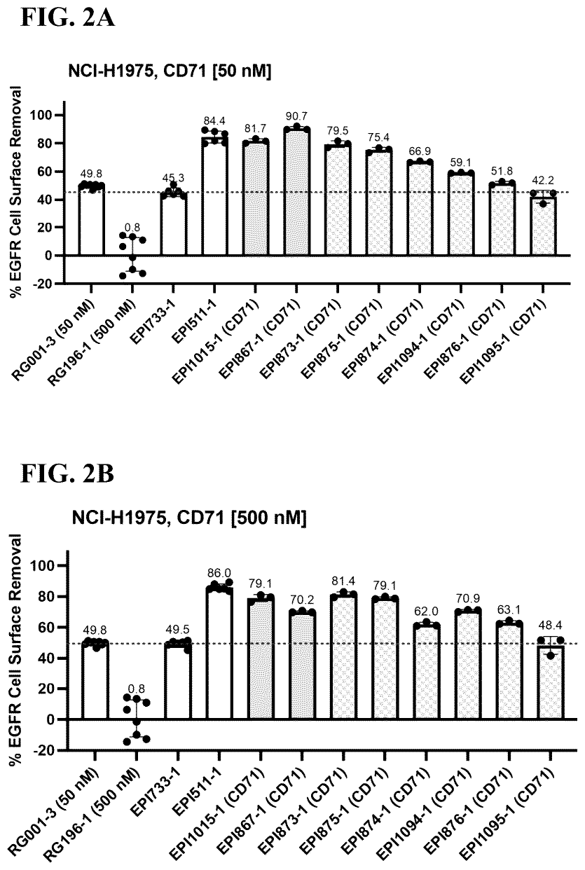

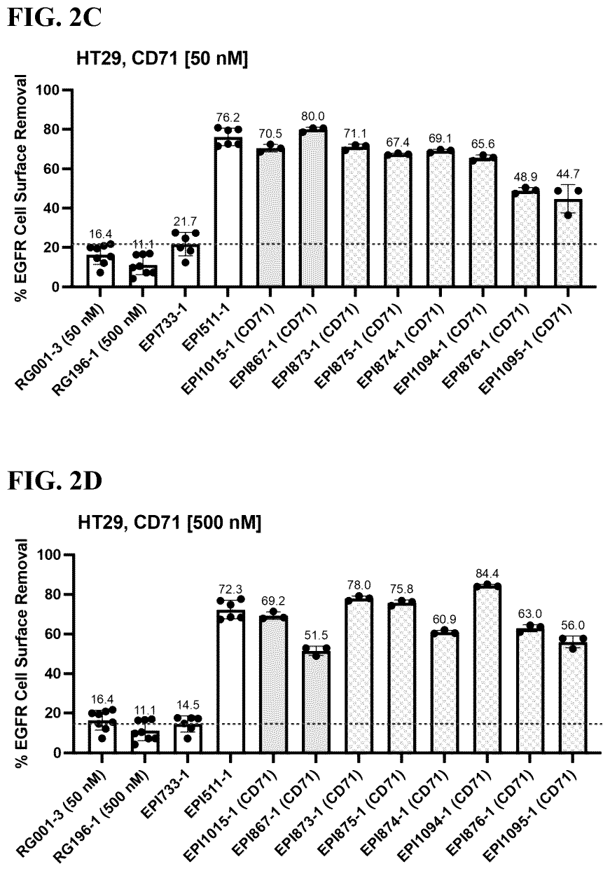

To determine the EGFR cell surface removal of bispecifics that bind to EGFR and a degrader protein, cell surface removal assays were conducted using EGFRxCD71 bispecific antibodies (antibodies that bind to EGFR and CD71; FIGS. 2A-2D). All EGFR-targeting bispecifics had Mav2 as the EGFR binding domain. The CD71 binding domain was varied to determine the effects of characteristics on EGFR degradation, such as the CD71 epitope or binding affinity of the antibody to CD71. The CD71 binding domains tested include EPI511-1 (ABBV2029 (EPKSC)), EPI1015-1 (ABBV2029 (GGGGS)), EPI867-1(AF-20D), EPI873-1 (h15G11v5), EPI874-1 (h15G11v5-52A), EPI875-1 (h15G11v5-53A), EPI876-1 (h15G11v5-53A/92A), EPI1094-1 (h15G11v5-92A), and EPI1095-1 (h15G11v5-52A/92A) (Table 5). The binding affinity of the monovalent binding domains to CD71 ranged from about 1 nM to about 1800 nM, as described in Singh et al., Mol Cancer Ther (2022) 21 (8): 1326-1336 (EPI511 and EPI1015) and US20210087288A1(EP1873, EP1874, and EP11094).

TABLE 5
Test EGFR × CD71 bispecific construct information

| | | | Monovalent | | |
|---|---|---|---|---|---|
| Molecule | EGFR | | KD to | Transferrin | |
| ID | Binder | CD71 Binder | CD71 (nM) | blocking? | Epitope |
| EPI511-1 | Mav2 | ABBV2029 (EPKSC) | 1 | Yes | Tf-site |
| EPI1015-1 | Mav2 | ABBV2029 | 1 | Yes | Tf-site |
| | | (GGGGS) | | | |
| EPI867-1 | Mav2 | AF-20D | unknown | Yes | Tf- |
| | | | | | site (glyco) |
| EPI873-1 | Mav2 | h15G11v5 | 7 | No | Apical |
| EPI874-1 | Mav2 | h15G11v5-52A | 44 | No | Apical |
| EPI875-1 | Mav2 | h15G11v5-53A | 13 | No | Apical |
| EPI876-1 | Mav2 | h15G11v5-53A/92A | weakened | No | Apical |
| | | | affinity | | |
| EPI1094-1 | Mav2 | h15G11v5-92A | 300 | No | Apical |
| EPI1095-1 | Mav2 | h15G11v5-52A/92A | weakened | No | Apical |
| | | | affinity | | |

Additionally, Cetuximab (RG001-3) was tested as an EGFR-only control. An IgGI isotype control against EGFR (RG196-1) was used as a non-targeted control. A palivizumab/Mav2 (RSV x EGFR bispecific) (EPI733-1) was tested as a single-arm Mav2 control with a second arm that did not bind to the target cell. Palivizumab/Mav2 was a baseline for comparison when determining efficacy of the other tested constructs.

TABLE 6
EGFR molecule information

| Molecule ID | Name | Arm 1 Binder | Arm 2 Binder | Purpose |
|---|---|---|---|---|
| RG001-3 | Cetuximab (MCE) | EGFR | (IgG) | SoC control |
| RG196-1 | Human IgG1 | | | Negative control |
| | Isotype Control | | | |
| | (BioXcell) | | | |
| EPI733-1 | Palivizumab/Mav2 | EGFR (Mav2) | RSV F | Single-arm Mav2 |
| | | | | control |

The various constructs were tested in the non-small cell lung cancer cell line NCIH1975 (FIGS. 2A and 2B) or colorectal cancer cell lines HT29 (FIGS. 2C and 2D) at 50 nM and 500 nM concentrations. In these assays, NCIH1975 or HT29 cells were seeded in 96-well plates and incubated overnight at 37 C and 5% CO₂. The next morning, cells were treated with either 50 or 500 nM of antibody. After 24 hours of treatment, cells were harvested using a dissociation reagent, stained using a fluorescently labeled anti-EGFR antibody, and acquired on a Cytek Northern Lights flow cytometer. Percent EGFR cell surface removal was calculated using an untreated control sample after accounting for background with an isotype control.

The EGFR x CD71 bispecific antibodies induced removal of EGFR from the cell surface in NCIH1975 cells at 50 nM (FIG. 2A) and at 500 nM (FIG. 2B), and in HT29 cells at 50 nM (FIG. 2C) and at 500 nM (FIG. 2D). The EGFR x CD71 bispecifics had higher levels of EGFR cell surface removal than Cetuximab, a standard of care molecule, and Palivizumab by the single arm Mav2 antibody across multiple cell lines (NCIH1975 and HT29). This effect is durable across molecular attributes including binding affinities ranging from about 1 to about 1800 nM and multiple epitopes. For example, trasferrin receptor non-blocking EPI876-1 (Mav2 x h15G11v5-53A/92A) bispecifics has a Kd of about 600 nM and exhibited similar cell surface removal to EPI511-1(Mav2 x ABBV2029) bispecific which has a Kd of about 1 nM indicating that EpiTACs with degrader arms having different affinities and epitope binding can drive similar target degradation.

This demonstrates the potential to tune the activity of the bispecific antibodies by varying the binder epitope and/or the binder affinity. Furthermore, this data indicates the effectiveness of bispecifics that bind to both EGFR and a degrader protein to remove EGFR from the surface of a target cell in multiple contexts.

### Example 8—EGFR Cell Surface Removal Screen to Identify Effective Degrader Protein Binding Domains for EGFR-Targeting Bispecific Antibodies

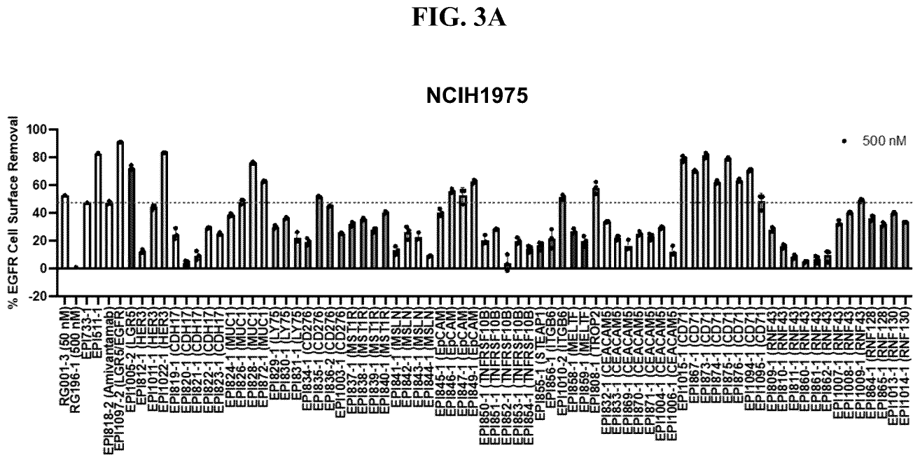

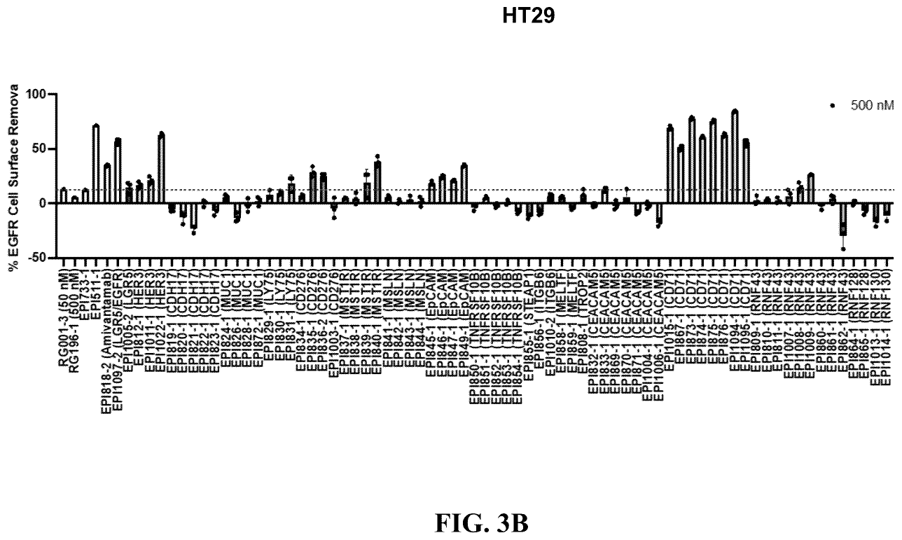

To identify degrader protein binding domains on EGFR-targeting bispecifics that resulted in high EGFR cell surface removal, a screen was preformed using 72 bispecifics (FIGS. 3A-3B). The 72 bispecifics bound to 20 unique degrader proteins. For most degrader proteins, multiple binding domains that bind to different epitopes were tested. Additionally, Cetuximab (RG001-3), an IgGI isotype control (RG196-1), and EGFR x RSV (EPI733-1) were tested as an EGFR only control, a negative control, and a single-arm Mav2 control, respectively, with EGFR x RSV used as a baseline for comparison. In this example, R001-3 corresponds to EPI431(Cetuximab commercial (MedChemExpress)) and RG196 corresponds to EPI1102 (Human IgGI isotype control commercial (Bio X Cell)). Positive controls for the assay included EGFR x cMet (EPI818, Amivantimab) and EGFR x LGR5 (EPI1097), along with a EGFR x CD71 molecule (EPI511-1) used as a control to track inter-assay variability. The bispecifics used in this example comprise a binding arm comprising the sequences listed in Table 7.

TABLE 7
Binding arm 1 targets and sequences

| | | SEQ | | SEQ | | SEQ | | SEQ | |
|---|---|---|---|---|---|---|---|---|---|
| | Arm 1 | ID | | ID | | ID | | ID | |
| ID | Target | NO | HC sequence | NO | VH sequence | NO | LC sequence | NO | VL sequence |
| EPI733 | RSV F | 9 | QVTLRESGPALVKPTQTL | 10 | QVTLRESGPA | 11 | DIQMTQSPS | 12 | DIQMTQSPS |
| | Protein | | TLTCTFSGFSLSTSGMSVG | | LVKPTQTLTL | | TLSASVGDRV | | TLSASVGDR |
| | | | WIRQPPGKALEWLADIW | | TCTFSGFSLST | | TITCKCQLSV | | VTITCKCQL |
| | | | WDDKKDYNPSLKSRLTIS | | SGMSVGWIR | | GYMHWYQQ | | SVGYMHW |
| | | | KDTSKNQVVLKVTNMDP | | QPPGKALEW | | KPGKAPKLLI | | YQQKPGKA |
| | | | ADTATYYCARSMITNWYF | | LADIWWDDK | | YDTSKLASGV | | PKLLIYDTS |
| | | | DVWGAGTTVTVSSASTKG | | KDYNPSLKSR | | PSRFSGSGSG | | KLASGVPSR |
| | | | PSVFPLAPSSKSTSGGTAA | | LTISKDTSKN | | TEFTLTISSLQ | | FSGSGSGTE |
| | | | LGCLVKDYFPEPVTVSWN | | QVVLKVTNM | | PDDFATYYCF | | FTLTISSLQP |
| | | | SGALTSGVHTFPAVLQSSG | | DPADTATYY | | QGSGYPFTFG | | DDFATYYC |
| | | | LYSLSSVVTVPSSSLGTQT | | CARSMITNW | | GGTKLEIKRT | | FQGSGYPFT |
| | | | YICNVNHKPSNTKVDKKV | | YFDVWGAGT | | VAAPSVFIFPP | | FGGGTKLEI |
| | | | EPKSCDKTHTCPPCPAPEL | | TVTVSS | | SDEQLKSGTA | | K |
| | | | LGGPSVFLFPPKPKDTLMI | | | | SVVCLLNNFY | | |
| | | | SRTPEVTCVVVDVSHEDP | | | | PREAKVQWK | | |
| | | | EVKFNWYVDGVEVHNAK | | | | VDNALQSGN | | |
| | | | TKPREEQYNSTYRVVSVL | | | | SQESVTEQDS | | |
| | | | TVLHQDWLNGKEYKCKV | | | | KDSTYSLSST | | |
| | | | SNKALPAPIEKTISKAKGQ | | | | LTLSKADYEK | | |
| | | | PREPQVYTLPPSRDELTKN | | | | HKVYACEVT | | |
| | | | QVSLWCLVKGFYPSDIAV | | | | HQGLSSPVTK | | |
| | | | EWESNGQPENNYKTTPPV | | | | SFNRGEC | | |
| | | | LDSDGSFFLYSKLTVDKSR | | | | | | |
| | | | WQQGNVFSCSVMHEALH | | | | | | |
| | | | NHYTQKSLSLSPGKGGSH | | | | | | |
| | | | HHHHH | | | | | | |
| EPI511 | CD71 | 558 | QVQLVQSGAEVKKPGASV | 559 | QVQLVQSG | 560 | DIQMTQSPSS | 561 | DIQMTQSP |
| | | | KMSCKASGYTFTSYWMH | | AEVKKPGAS | | LSASVGDRVT | | SSLSASVG |
| | | | WVRQAPGQGLEWIGAIYP | | VKMSCKAS | | ITCSASSSVYY | | DRVTITCS |
| | | | GNSETGYAQKFQGRATLT | | GYTFTSYW | | MYWFQQKPG | | ASSSVYY |
| | | | ADTSTSTAYMELSSLRSED | | MHWVRQAP | | KAPKLWIYST | | MYWFQQK |
| | | | TAVYYCTRENWDPGFAF | | GQGLEWIG | | SNLASGVPSR | | PGKAPKL |
| | | | WGQGTLITVSSASTKGPSV | | AIYPGNSET | | FSGSGSGTDY | | WIYSTSNL |
| | | | FPLAPSSKSTSGGTAALGC | | GYAQKFQG | | TLTISSMQPE | | ASGVPSRF |
| | | | LVKDYFPEPVTVSWNSGA | | RATLTADTS | | DFATYYCQQ | | SGSGSGTD |
| | | | LTSGVHTFPAVLQSSGLYS | | TSTAYMELS | | RRNYPYTFGQ | | YTLTISSM |
| | | | LSSVVTVPSSSLGTQTYIC | | SLRSEDTAV | | GTKLEIKRTV | | QPEDFATY |
| | | | NVNHKPSNTKVDKKVEPK | | YYCTRENW | | AAPSVFIFPPS | | YCQQRRN |
| | | | SCDKTHTCPPCPAPELLGG | | DPGFAFWG | | DEQLKSGTAS | | YPYTFGQ |
| | | | PSVFLFPPKPKDTLMISRTP | | QGTLITVSS | | VVCLLNNFYP | | GTKLEIK |
| | | | EVTCVVVDVSHEDPEVKF | | | | REAKVQWKV | | |
| | | | NWYVDGVEVHNAKTKPR | | | | DNALQSGNS | | |
| | | | EEQYNSTYRVVSVLTVLH | | | | QESVTEQDSK | | |
| | | | QDWLNGKEYKCKVSNKA | | | | DSTYSLSSTLT | | |
| | | | LPAPIEKTISKAKGQPREPQ | | | | LSKADYEKH | | |
| | | | VYTLPPSRDELTKNQVSL | | | | KVYACEVTH | | |
| | | | WCLVKGFYPSDIAVEWES | | | | QGLSSPVTKS | | |
| | | | NGQPENNYKTTPPVLDSD | | | | FNRGEC | | |
| | | | GSFFLYSKLTVDKSRWQQ | | | | | | |
| | | | GNVFSCSVMHEALHNHYT | | | | | | |
| | | | QKSLSLSPGK | | | | | | |
| EPI818 | MET | 1 | QVQLVQSGAEVKKPGAS | 2 | QVQLVQSGA | 3 | DIQMTQSPS | 4 | DIQMTQSPS |
| | | | VKVSCETSGYTFTSYGISW | | EVKKPGASV | | SVSASVGDRV | | SVSASVGDR |
| | | | VRQAPGHGLEWMGWISA | | KVSCETSGYT | | TITCRASQGIS | | VTITCRASQ |
| | | | YNGYTNYAQKLQGRVTM | | FTSYGISWVR | | NWLAWFQHK | | GISNWLAW |
| | | | TTDTSTSTAYMELRSLRSD | | QAPGHGLEW | | PGKAPKLLIY | | FQHKPGKA |
| | | | DTAVYYCARDLRGTNYFD | | MGWISAYNG | | AASSLLSGVP | | PKLLIYAAS |
| | | | YWGQGTLVTVSSASTKGP | | YTNYAQKLQ | | SRFSGSGSGT | | SLLSGVPSR |
| | | | SVFPLAPSSKSTSGGTAAL | | GRVTMTTDTS | | DFTLTISSLQP | | FSGSGSGTD |
| | | | GCLVKDYFPEPVTVSWNS | | TSTAYMELRS | | EDFATYYCQ | | FTLTISSLQP |
| | | | GALTSGVHTFPAVLQSSGL | | LRSDDTAVY | | QANSFPITFG | | EDFATYYC |
| | | | YSLSSVVTVPSSSLGTQTYI | | YCARDLRGT | | QGTRLEIKRT | | QQANSFPIT |
| | | | CNVNHKPSNTKVDKKVEP | | NYFDYWGQG | | VAAPSVFIFPP | | FGQGTRLEI |
| | | | KSCDKTHTCPPCPAPELLG | | TLVTVSS | | SDEQLKSGTA | | K |
| | | | GPSVFLFPPKPKDTLMISR | | | | SVVCLLNNFY | | |
| | | | TPEVTCVVVDVSHEDPEV | | | | PREAKVQWK | | |
| | | | KFNWYVDGVEVHNAKTK | | | | VDNALQSGN | | |
| | | | PREEQYNSTYRVVSVLTV | | | | SQESVTEQDS | | |
| | | | LHQDWLNGKEYKCKVSN | | | | KDSTYSLSST | | |
| | | | KALPAPIEKTISKAKGQPR | | | | LTLSKADYEK | | |
| | | | EPQVYTLPPSRDELTKNQV | | | | HKVYACEVT | | |
| | | | SLWCLVKGFYPSDIAVEW | | | | HQGLSSPVTK | | |
| | | | ESNGQPENNYKTTPPVLDS | | | | SFNRGEC | | |
| | | | DGSFFLYSKLTVDKSRWQ | | | | | | |
| | | | QGNVFSCSVMHEALHNH | | | | | | |
| | | | YTQKSLSLSPGK | | | | | | |
| EPI1097 | LGR5 | 5 | EVQLVQSGSKLKKPGASV | 6 | EVQLVQSGSK | 7 | DIQMTQSPS | 8 | DIQMTQSPS |
| | | | KVSCKASGYTFTSYTMNW | | LKKPGASVK | | SLSASVGDRV | | SLSASVGDR |
| | | | VRQAPGQGLEWMGWINT | | VSCKASGYTF | | TITCRASQSIS | | VTITCRASQ |
| | | | DTGDPTYAQGFTGRFVFS | | TSYTMNWVR | | SYLNWYQQK | | SISSYLNWY |
| | | | LDTSVSTAFLQINSLKAED | | QAPGQGLEW | | PGKAPKLLIY | | QQKPGKAP |
| | | | TAVYYCARGDCDSTSCYR | | MGWINTDTG | | AASSLQSGVP | | KLLIYAASS |
| | | | YSYGYEDYWGQGTLVTV | | DPTYAQGFTG | | SRFSGSGSGT | | LQSGVPSRF |
| | | | SSASTKGPSVFPLAPSSKST | | RFVFSLDTSV | | DFTLTISSLQP | | SGSGSGTDF |
| | | | SGGTAALGCLVKDYFPEP | | STAFLQINSLK | | EDFATYYCQ | | TLTISSLQPE |
| | | | VTVSWNSGALTSGVHTFP | | AEDTAVYYC | | QSYSTPPTFG | | DFATYYCQ |
| | | | AVLQSSGLYSLSSVVTVPS | | ARGDCDSTSC | | QGTKVEIKRT | | QSYSTPPTF |
| | | | SSLGTQTYICNVNHKPSNT | | YRYSYGYED | | VAAPSVFIFPP | | GQGTKVEIK |
| | | | KVDKKVEPKSCDKTHTCP | | YWGQGTLVT | | SDEQLKSGTA | | |
| | | | PCPAPELLGGPSVFLFPPKP | | VSS | | SVVCLLNNFY | | |
| | | | KDTLMISRTPEVTCVVVD | | | | PREAKVQWK | | |
| | | | VSHEDPEVKFNWYVDGV | | | | VDNALQSGN | | |
| | | | EVHNAKTKPREEQYNSTY | | | | SQESVTEQDS | | |
| | | | RVVSVLTVLHQDWLNGK | | | | KDSTYSLSST | | |
| | | | EYKCKVSNKALPAPIEKTI | | | | LTLSKADYEK | | |
| | | | SKAKGQPREPQVYTLPPSR | | | | HKVYACEVT | | |
| | | | DELTKNQVSLWCLVKGFY | | | | HQGLSSPVTK | | |
| | | | PSDIAVEWESNGQPENNY | | | | SFNRGEC | | |
| | | | KTTPPVLDSDGSFFLYSKL | | | | | | |
| | | | TVDKSRWQQGNVFSCSV | | | | | | |
| | | | MHEALHNHYTQKSLSLSPGK | | | | | | |
| EPI1005 | LGR5 | 266 | EVQLVQSGSKLKKPGASV | 267 | EVQLVQSGSK | 268 | DIQMTQSPS | 269 | DIQMTQSPS |
| | | | KVSCKASGYTFTSYTMNW | | LKKPGASVK | | SLSASVGDRV | | SLSASVGDR |
| | | | VRQAPGQGLEWMGWINT | | VSCKASGYTF | | TITCRASQSIS | | VTITCRASQ |
| | | | DTGDPTYAQGFTGRFVFS | | TSYTMNWVR | | SYLNWYQQK | | SISSYLNWY |
| | | | LDTSVSTAFLQINSLKAED | | QAPGQGLEW | | PGKAPKLLIY | | QQKPGKAP |
| | | | TAVYYCARGDCDSTSCYR | | MGWINTDTG | | AASSLQSGVP | | KLLIYAASS |
| | | | YSYGYEDYWGQGTLVTV | | DPTYAQGFTG | | SRFSGSGSGT | | LQSGVPSRF |
| | | | SSASTKGPSVFPLAPSSKST | | RFVFSLDTSV | | DFTLTISSLQP | | SGSGSGTDF |
| | | | SGGTAALGCLVKDYFPEP | | STAFLQINSLK | | EDFATYYCQ | | TLTISSLQPE |
| | | | VTVSWNSGALTSGVHTFP | | AEDTAVYYC | | QSYSTPPTFG | | DFATYYCQ |
| | | | AVLQSSGLYSLSSVVTVPS | | ARGDCDSTSC | | QGTKVEIKRT | | QSYSTPPTF |
| | | | SSLGTQTYICNVNHKPSNT | | YRYSYGYED | | VAAPSVFIFPP | | GQGTKVEIK |
| | | | KVDKKVEPKSCDKTHTCP | | YWGQGTLVT | | SDEQLKSGTA | | |
| | | | PCPAPELLGGPSVFLFPPKP | | VSS | | SVVCLLNNFY | | |
| | | | KDTLMISRTPEVTCVVVD | | | | PREAKVQWK | | |
| | | | VSHEDPEVKFNWYVDGV | | | | VDNALQSGN | | |
| | | | EVHNAKTKPREEQYNSTY | | | | SQESVTEQDS | | |
| | | | RVVSVLTVLHQDWLNGK | | | | KDSTYSLSST | | |
| | | | EYKCKVSNKALPAPIEKTI | | | | LTLSKADYEK | | |
| | | | SKAKGQPREPQVYTLPPSR | | | | HKVYACEVT | | |
| | | | DELTKNQVSLWCLVKGFY | | | | HQGLSSPVTK | | |
| | | | PSDIAVEWESNGQPENNY | | | | SFNRGEC | | |
| | | | KTTPPVLDSDGSFFLYSKL | | | | | | |
| | | | TVDKSRWQQGNVFSCSV | | | | | | |
| | | | MHEALHNHYTQKSLSLSPGK | | | | | | |
| EPI812 | HER3 | 30 | EVQLLESGGGLVQPGGSL | 31 | EVQLLESGGG | 32 | DIQMTQSPS | 33 | DIQMTQSPS |
| | | | RLSCAASGFTFSSYAMSW | | LVQPGGSLRL | | SLSASVGDRV | | SLSASVGDR |
| | | | VRQAPGKGLEWVSAINSQ | | SCAASGFTFS | | TITCRASQGIS | | VTITCRASQ |
| | | | GKSTYYADSVKGRFTISRD | | SYAMSWVRQ | | NWLAWYQQ | | GISNWLAW |
| | | | NSKNTLYLQMNSLRAEDT | | APGKGLEWV | | KPGKAPKLLI | | YQQKPGKA |
| | | | AVYYCARWGDEGFDIWG | | SAINSQGKST | | YGASSLQSGV | | PKLLIYGAS |
| | | | QGTLVTVSSASTKGPSVFP | | YYADSVKGR | | PSRFSGSGSG | | SLQSGVPSR |
| | | | LAPSSKSTSGGTAALGCLV | | FTISRDNSKN | | TDFTLTISSLQ | | FSGSGSGTD |
| | | | KDYFPEPVTVSWNSGALT | | TLYLQMNSLR | | PEDFATYYCQ | | FTLTISSLQP |
| | | | SGVHTFPAVLQSSGLYSLS | | AEDTAVYYC | | QYSSFPTTFG | | EDFATYYC |
| | | | SVVTVPSSSLGTQTYICNV | | ARWGDEGFDI | | QGTKVEIKRT | | QQYSSFPTT |
| | | | NHKPSNTKVDKRVEPKSC | | WGQGTLVTV | | VAAPSVFIFPP | | FGQGTKVEI |
| | | | DKTHTCPPCPAPELLGGPS | | SS | | SDEQLKSGTA | | K |
| | | | VFLFPPKPKDTLMISRTPE | | | | SVVCLLNNFY | | |
| | | | VTCVVVDVSHEDPEVKFN | | | | PREAKVQWK | | |
| | | | WYVDGVEVHNAKTKPRE | | | | VDNALQSGN | | |
| | | | EQYNSTYRVVSVLTVLHQ | | | | SQESVTEQDS | | |
| | | | DWLNGKEYKCKVSNKAL | | | | KDSTYSLSST | | |
| | | | PAPIEKTISKAKGQPREPQ | | | | LTLSKADYEK | | |
| | | | VYTLPPSRDELTKNQVSL | | | | HKVYACEVT | | |
| | | | WCLVKGFYPSDIAVEWES | | | | HQGLSSPVTK | | |
| | | | NGQPENNYKTTPPVLDSD | | | | SFNRGEC | | |
| | | | GSFFLYSKLTVDKSRWQQ | | | | | | |
| | | | GNVFSCSVMHEALHNHYT | | | | | | |
| | | | QKSLSLSPGK | | | | | | |
| EPI1011 | HER3 | 290 | QVQLQQWGAGLLKPSETL | 291 | QVQLQQWGA | 292 | DIEMTQSPD | 293 | DIEMTQSPD |
| | | | SLTCAVYGGSFSGYYWSW | | GLLKPSETLS | | SLAVSLGERA | | SLAVSLGER |
| | | | IRQPPGKGLEWIGEINHSG | | LTCAVYGGSF | | TINCRSSQSVL | | ATINCRSSQ |
| | | | STNYNPSLKSRVTISVETS | | SGYYWSWIR | | YSSSNRNYLA | | SVLYSSSNR |
| | | | KNQFSLKLSSVTAADTAV | | QPPGKGLEWI | | WYQQNPGQP | | NYLAWYQQ |
| | | | YYCARDKWTWYFDLWG | | GEINHSGSTN | | PKLLIYWAST | | NPGQPPKLL |
| | | | RGTLVTVSSASTKGPSVFP | | YNPSLKSRVT | | RESGVPDRFS | | IYWASTRES |
| | | | LAPSSKSTSGGTAALGCLV | | ISVETSKNQFS | | GSGSGTDFTL | | GVPDRFSGS |
| | | | KDYFPEPVTVSWNSGALT | | LKLSSVTAAD | | TISSLQAEDV | | GSGTDFTLT |
| | | | SGVHTFPAVLQSSGLYSLS | | TAVYYCARD | | AVYYCQQYY | | ISSLQAEDV |
| | | | SVVTVPSSSLGTQTYICNV | | KWTWYFDL | | STPRTFGQGT | | AVYYCQQY |
| | | | NHKPSNTKVDKRVEPKSC | | WGRGTLVTV | | KVEIKRTVAA | | YSTPRTFGQ |
| | | | DKTHTCPPCPAPELLGGPS | | SS | | PSVFIFPPSDE | | GTKVEIK |
| | | | VFLFPPKPKDTLMISRTPE | | | | QLKSGTASVV | | |
| | | | VTCVVVDVSHEDPEVKFN | | | | CLLNNFYPRE | | |
| | | | WYVDGVEVHNAKTKPRE | | | | AKVQWKVDN | | |
| | | | EQYNSTYRVVSVLTVLHQ | | | | ALQSGNSQES | | |
| | | | DWLNGKEYKCKVSNKAL | | | | VTEQDSKDST | | |
| | | | PAPIEKTISKAKGQPREPQ | | | | YSLSSTLTLSK | | |
| | | | VYTLPPSRDELTKNQVSL | | | | ADYEKHKVY | | |
| | | | WCLVKGFYPSDIAVEWES | | | | ACEVTHQGLS | | |
| | | | NGQPENNYKTTPPVLDSD | | | | SPVTKSFNRG | | |
| | | | GSFFLYSKLTVDKSR WQQ | | | | EC | | |
| | | | GNVFSCSVMHEALHNHYT | | | | | | |
| | | | QKSLSLSPGKGGSHHHHHH | | | | | | |
| EPI1022 | HER3 | 306 | EVQLVESGGGLVQPGGSL | 307 | EVQLVESGGG | 308 | DIQMTQSPS | 309 | DIQMTQSPS |
| | | | RLSCAASGFTLSGDWIHW | | LVQPGGSLRL | | SLSASVGDRV | | SLSASVGDR |
| | | | VRQAPGKGLEWVGEISAA | | SCAASGFTLS | | TITCRASQNIA | | VTITCRASQ |
| | | | GGYTDYADSVKGRFTISA | | GDWIHWVRQ | | TDVAWYQQK | | NIATDVAW |
| | | | DTSKNTAYLQMNSLRAED | | APGKGLEWV | | PGKAPKLLIY | | YQQKPGKA |
| | | | TAVYYCARESRVSFEAAM | | GEISAAGGYT | | SASFLYSGVP | | PKLLIYSASF |
| | | | DYWGQGTLVTVSSASTKG | | DYADSVKGR | | SRFSGSGSGT | | LYSGVPSRF |
| | | | PSVFPLAPSSKSTSGGTAA | | FTISADTSKNT | | DFTLTISSLQP | | SGSGSGTDF |
| | | | LGCLVKDYFPEPVTVSWN | | AYLQMNSLR | | EDFATYYCQ | | TLTISSLQPE |
| | | | SGALTSGVHTFPAVLQSSG | | AEDTAVYYC | | QSEPEPYTFG | | DFATYYCQ |
| | | | LYSLSSVVTVPSSSLGTQT | | ARESRVSFEA | | QGTKVEIKRT | | QSEPEPYTF |
| | | | YICNVNHKPSNTKVDKKV | | AMDYWGQG | | VAAPSVFIFPP | | GQGTKVEIK |
| | | | EPKSCDKTHTCPPCPAPEL | | TLVTVSS | | SDEQLKSGTA | | |
| | | | LGGPSVFLFPPKPKDTLMI | | | | SVVCLLNNFY | | |
| | | | SRTPEVTCVVVDVSHEDP | | | | PREAKVQWK | | |
| | | | EVKFNWYVDGVEVHNAK | | | | VDNALQSGN | | |
| | | | TKPREEQYNSTYRVVSVL | | | | SQESVTEQDS | | |
| | | | TVLHQDWLNGKEYKCKV | | | | KDSTYSLSST | | |
| | | | SNKALPAPIEKTISKAKGQ | | | | LTLSKADYEK | | |
| | | | PREPQVYTLPPSRDELTKN | | | | HKVYACEVT | | |
| | | | QVSLWCLVKGFYPSDIAV | | | | HQGLSSPVTK | | |
| | | | EWESNGQPENNYKTTPPV | | | | SFNRGEC | | |
| | | | LDSDGSFFLYSKLTVDKSR | | | | | | |
| | | | WQQGNVFSCSVMHEALH | | | | | | |
| | | | NHYTQKSLSLSPGK | | | | | | |
| EPI819 | CDH17 | 34 | EVQLVESGGGLVQPGGSL | 35 | EVQLVESGGG | 36 | DIQMTQSPS | 37 | DIQMTQSPS |
| | | | RLSCAASGFTFSSYAMSW | | LVQPGGSLRL | | SLSASVGDRV | | SLSASVGDR |
| | | | VRQTPGKGLEWVAVIDSN | | SCAASGFTFS | | TITCRASQDIS | | VTITCRASQ |
| | | | GGSTYYPDTVKDRFTISRD | | SYAMSWVRQ | | GYLNWLQQK | | DISGYLNWL |
| | | | NSKNTLYLQMNSLRAEDT | | TPGKGLEWV | | PGGAIKRLIYT | | QQKPGGAIK |
| | | | AVYYCSSYTNLGAYWGQ | | AVIDSNGGST | | TSTLDSGVPK | | RLIYTTSTL |
| | | | GTLVTVSAASTKGPSVFPL | | YYPDTVKDRF | | RFSGSGSGTD | | DSGVPKRFS |
| | | | APSSKSTSGGTAALGCLV | | TISRDNSKNT | | FTLTISSLQSE | | GSGSGTDFT |
| | | | KDYFPEPVTVSWNSGALT | | LYLQMNSLR | | DFATYYCLQ | | LTISSLQSED |
| | | | SGVHTFPAVLQSSGLYSLS | | AEDTAVYYC | | YASSPFTFGG | | FATYYCLQ |
| | | | SVVTVPSSSLGTQTYICNV | | SSYTNLGAY | | GTKVEIKRTV | | YASSPFTFG |
| | | | NHKPSNTKVDKKVEPKSC | | WGQGTLVTV | | AAPSVFIFPPS | | GGTKVEIK |
| | | | DKTHTCPPCPAPELLGGPS | | SA | | DEQLKSGTAS | | |
| | | | VFLFPPKPKDTLMISRTPE | | | | VVCLLNNFYP | | |
| | | | VTCVVVDVSHEDPEVKFN | | | | REAKVQWKV | | |
| | | | WYVDGVEVHNAKTKPRE | | | | DNALQSGNS | | |
| | | | EQYNSTYRVVSVLTVLHQ | | | | QESVTEQDSK | | |
| | | | DWLNGKEYKCKVSNKAL | | | | DSTYSLSSTLT | | |
| | | | PAPIEKTISKAKGQPREPQ | | | | LSKADYEKH | | |
| | | | VYTLPPSRDELTKNQVSL | | | | KVYACEVTH | | |
| | | | WCLVKGFYPSDIAVEWES | | | | QGLSSPVTKS | | |
| | | | NGQPENNYKTTPPVLDSD | | | | FNRGEC | | |
| | | | GSFFLYSKLTVDKSRWQQ | | | | | | |
| | | | GNVFSCSVMHEALHNHYT | | | | | | |
| | | | QKSLSLSPGK | | | | | | |
| EPI820 | CDH17 | 38 | QVQLVESGGGVVQPGRSL | 39 | QVQLVESGG | 40 | DIVMTQTPL | 41 | DIVMTQTPL |
| | | | RLSCAASGFTFSDYYMYW | | GVVQPGRSLR | | SLSVTPGQPA | | SLSVTPGQP |
| | | | VRQAPGKGLEWVASISFD | | LSCAASGFTF | | SISCRSSQSIV | | ASISCRSSQS |
| | | | GTYTYYTDRVKGRFTISR | | SDYYMYWVR | | HSNGNTYLE | | IVHSNGNTY |
| | | | DNSKNTLYLQMNSLRAED | | QAPGKGLEW | | WYLQKPGQS | | LEWYLQKP |
| | | | TAVYYCARDRPAWFPYW | | VASISFDGTY | | PQLLIYKVSN | | GQSPQLLIY |
| | | | GQGTLVTVSAASTKGPSV | | TYYTDRVKG | | RFSGVPDRFS | | KVSNRFSGV |
| | | | FPLAPSSKSTSGGTAALGC | | RFTISRDNSK | | GSGSGTDFTL | | PDRFSGSGS |
| | | | LVKDYFPEPVTVSWNSGA | | NTLYLQMNS | | KISRVEAEDV | | GTDFTLKIS |
| | | | LTSGVHTFPAVLQSSGLYS | | LRAEDTAVY | | GVYYCFQGS | | RVEAEDVG |
| | | | LSSVVTVPSSSLGTQTYIC | | YCARDRPAW | | HVPLTFGAGT | | VYYCFQGS |
| | | | NVNHKPSNTKVDKKVEPK | | FPYWGQGTL | | KLELKRTVAA | | HVPLTFGAG |
| | | | SCDKTHTCPPCPAPELLGG | | VTVSA | | PSVFIFPPSDE | | TKLELK |
| | | | PSVFLFPPKPKDTLMISRTP | | | | QLKSGTASVV | | |
| | | | EVTCVVVDVSHEDPEVKF | | | | CLLNNFYPRE | | |
| | | | NWYVDGVEVHNAKTKPR | | | | AKVQWKVDN | | |
| | | | EEQYNSTYRVVSVLTVLH | | | | ALQSGNSQES | | |
| | | | QDWLNGKEYKCKVSNKA | | | | VTEQDSKDST | | |
| | | | LPAPIEKTISKAKGQPREPQ | | | | YSLSSTLTLSK | | |
| | | | VYTLPPSRDELTKNQVSL | | | | ADYEKHKVY | | |
| | | | WCLVKGFYPSDIAVEWES | | | | ACEVTHQGLS | | |
| | | | NGQPENNYKTTPPVLDSD | | | | SPVTKSFNRG | | |
| | | | GSFFLYSKLTVDKSR WQQ | | | | EC | | |
| | | | GNVFSCSVMHEALHNHYT | | | | | | |
| | | | QKSLSLSPGK | | | | | | |
| EPI821 | CDH17 | 42 | EVQLLETGGGVVKPGGSL | 43 | EVQLLETGGG | 44 | DVVLTQTPL | 45 | DVVLTQTPL |
| | | | KLSCAASGFTFSNYGMSW | | VVKPGGSLKL | | SLPVTLGDQA | | SLPVTLGDQ |
| | | | VRQTPEKRLEWVAAINRD | | SCAASGFTFS | | SISCRSSQSLL | | ASISCRSSQS |
| | | | GGTTYYTDNVKGRFTISR | | NYGMSWVRQ | | HSNGNTYLH | | LLHSNGNTY |
| | | | DNAKNSLYLQMSSLRSED | | TPEKRLEWV | | WYLLKPGQSP | | LHWYLLKP |
| | | | TALYYCARQFLLWDGWY | | AAINRDGGTT | | KLLIYKVSNR | | GQSPKLLIY |
| | | | FDVWGAGTTVTVSSASTK | | YYTDNVKGR | | FSGVPDRFSG | | KVSNRFSGV |
| | | | GPSVFPLAPSSKSTSGGTA | | FTISRDNAKN | | SGSGTDFTLKI | | PDRFSGSGS |
| | | | ALGCLVKDYFPEPVTVSW | | SLYLQMSSLR | | TRVEAEDLGV | | GTDFTLKIT |
| | | | NSGALTSGVHTFPAVLQSS | | SEDTALYYCA | | YFCSQSTHVL | | RVEAEDLG |
| | | | GLYSLSSVVTVPSSSLGTQ | | RQFLLWDGW | | TFGAGTKLEL | | VYFCSQSTH |
| | | | TYICNVNHKPSNTKVDKK | | YFDVWGAGT | | KRTVAAPSVF | | VLTFGAGTK |
| | | | VEPKSCDKTHTCPPCPAPE | | TVTVSS | | IFPPSDEQLKS | | LELK |
| | | | LLGGPSVFLFPPKPKDTLM | | | | GTASVVCLLN | | |
| | | | ISRTPEVTCVVVDVSHEDP | | | | NFYPREAKVQ | | |
| | | | EVKFNWYVDGVEVHNAK | | | | WKVDNALQS | | |
| | | | TKPREEQYNSTYRVVSVL | | | | GNSQESVTEQ | | |
| | | | TVLHQDWLNGKEYKCKV | | | | DSKDSTYSLS | | |
| | | | SNKALPAPIEKTISKAKGQ | | | | STLTLSKADY | | |
| | | | PREPQVYTLPPSRDELTKN | | | | EKHKVYACE | | |
| | | | QVSLWCLVKGFYPSDIAV | | | | VTHQGLSSPV | | |
| | | | EWESNGQPENNYKTTPPV | | | | TKSFNRGEC | | |
| | | | LDSDGSFFLYSKLTVDKSR | | | | | | |
| | | | WQQGNVFSCSVMHEALH | | | | | | |
| | | | NHYTQKSLSLSPGK | | | | | | |
| EPI822 | CDH17 | 46 | EVQLQQSVAELVKPGASV | 47 | EVQLQQSVAE | 48 | DIVMSQSPS | 49 | DIVMSQSPS |
| | | | KMSCKVSGYTLTDHTIHW | | LVKPGASVK | | SLAVSVGEKV | | SLAVSVGEK |
| | | | MKQRPEQGLEWIGYIYPR | | MSCKVSGYT | | TMSCKSSQSL | | VTMSCKSS |
| | | | DGITGYNEKFKGKATLTA | | LTDHTIHWM | | LHSSNQKNYL | | QSLLHSSNQ |
| | | | DTSSSTAYMQLNSLTSEDS | | KQRPEQGLE | | AWYQQKPGQ | | KNYLAWYQ |
| | | | AVYFCARWGYSYRNYAY | | WIGYIYPRDG | | SPKVLIYWAS | | QKPGQSPK |
| | | | YYDYWGQGTTLTVSSAST | | ITGYNEKFKG | | TRESGVPDRF | | VLIYWASTR |
| | | | KGPSVFPLAPSSKSTSGGT | | KATLTADTSS | | TGSGSGTDFT | | ESGVPDRFT |
| | | | AALGCLVKDYFPEPVTVS | | STAYMQLNSL | | LTITSVKSEDL | | GSGSGTDFT |
| | | | WNSGALTSGVHTFPAVLQ | | TSEDSAVYFC | | AVYYCQQYY | | LTITSVKSE |
| | | | SSGLYSLSSVVTVPSSSLG | | ARWGYSYRN | | SYPWTFGGGT | | DLAVYYCQ |
| | | | TQTYICNVNHKPSNTKVD | | YAYYYDYWG | | RLEIKRTVAA | | QYYSYPWT |
| | | | KKVEPKSCDKTHTCPPCP | | QGTTLTVSS | | PSVFIFPPSDE | | FGGGTRLEI |
| | | | APELLGGPSVFLFPPKPKD | | | | QLKSGTASVV | | K |
| | | | TLMISRTPEVTCVVVDVSH | | | | CLLNNFYPRE | | |
| | | | EDPEVKFNWYVDGVEVH | | | | AKVQWKVDN | | |
| | | | NAKTKPREEQYNSTYRVV | | | | ALQSGNSQES | | |
| | | | SVLTVLHQDWLNGKEYK | | | | VTEQDSKDST | | |
| | | | CKVSNKALPAPIEKTISKA | | | | YSLSSTLTLSK | | |
| | | | KGQPREPQVYTLPPSRDEL | | | | ADYEKHKVY | | |
| | | | TKNQVSLWCLVKGFYPSD | | | | ACEVTHQGLS | | |
| | | | IAVEWESNGQPENNYKTT | | | | SPVTKSFNRG | | |
| | | | PPVLDSDGSFFLYSKLTVD | | | | EC | | |
| | | | KSRWQQGNVFSCSVMHE | | | | | | |
| | | | ALHNHYTQKSLSLSPGK | | | | | | |
| EPI823 | CDH17 | 50 | QVQLVQSGAEVKKPGSSV | 51 | QVQLVQSGA | 52 | EIVMTQSPA | 53 | EIVMTQSPA |
| | | | KVSCKASGYTFSDHTIHW | | EVKKPGSSVK | | TLSVSPGERA | | TLSVSPGER |
| | | | VRQAPGQGLEWMGYIYPR | | VSCKASGYTF | | TLSCRASQSV | | ATLSCRASQ |
| | | | LGSTKYAEKFQGRVTITA | | SDHTIHWVRQ | | LYSSNQKQYL | | SVLYSSNQK |
| | | | DKSTSTAYMELSSLRSEDT | | APGQGLEWM | | AWYQQKPGQ | | QYLAWYQQ |
| | | | AVYYCARWGYYYGSSRY | | GYIYPRLGST | | APRLLIYGAS | | KPGQAPRLL |
| | | | YFDYWGQGTLVTVSSAST | | KYAEKFQGR | | TRETGIPARFS | | TYGASTRET |
| | | | KGPSVFPLAPSSKSTSGGT | | VTITADKSTS | | GSGSGTEFTL | | GIPARFSGS |
| | | | AALGCLVKDYFPEPVTVS | | TAYMELSSLR | | TISSLQSEDFA | | GSGTEFTLTI |
| | | | WNSGALTSGVHTFPAVLQ | | SEDTAVYYC | | VYYCQQYYS | | SSLQSEDFA |
| | | | SSGLYSLSSVVTVPSSSLG | | ARWGYYYGS | | YPWTFGQGT | | VYYCQQYY |
| | | | TQTYICNVNHKPSNTKVD | | SRYYFDYWG | | KLEIKRTVAA | | SYPWTFGQ |
| | | | KKVEPKSCDKTHTCPPCP | | QGTLVTVSS | | PSVFIFPPSDE | | GTKLEIK |
| | | | APELLGGPSVFLFPPKPKD | | | | QLKSGTASVV | | |
| | | | TLMISRTPEVTCVVVDVSH | | | | CLLNNFYPRE | | |
| | | | EDPEVKFNWYVDGVEVH | | | | AKVQWKVDN | | |
| | | | NAKTKPREEQYNSTYRVV | | | | ALQSGNSQES | | |
| | | | SVLTVLHQDWLNGKEYK | | | | VTEQDSKDST | | |
| | | | CKVSNKALPAPIEKTISKA | | | | YSLSSTLTLSK | | |
| | | | KGQPREPQVYTLPPSRDEL | | | | ADYEKHKVY | | |
| | | | TKNQVSLWCLVKGFYPSD | | | | ACEVTHQGLS | | |
| | | | IAVEWESNGQPENNYKTT | | | | SPVTKSFNRG | | |
| | | | PPVLDSDGSFFLYSKLTVD | | | | EC | | |
| | | | KSRWQQGNVFSCSVMHE | | | | | | |
| | | | ALHNHYTQKSLSLSPGK | | | | | | |
| EPI824 | MUC1 | 54 | EVQLVESGGGLVQPGGSM | 55 | EVQLVESGGG | 56 | DIVMTQSPL | 57 | DIVMTQSPL |
| | | | RLSCVASGFPFSNYWMN | | LVQPGGSMR | | SNPVTPGEPA | | SNPVTPGEP |
| | | | WVRQAPGKGLEWVGEIRL | | LSCVASGFPF | | SISCRSSKSLL | | ASISCRSSKS |
| | | | KSNQYTTHYAESVKGRFTI | | SNYWMNWV | | HSNGITYFFW | | LLHSNGITY |
| | | | SRDDSKNSLYLQMNSLKT | | RQAPGKGLE | | YLQKPGQSPQ | | FFWYLQKP |
| | | | EDTAVYYCTRHYYFDYW | | WVGEIRLKSN | | LLIYQMSNLA | | GQSPQLLIY |
| | | | GQGTLVTVSSASTKGPSVF | | QYTTHYAESV | | SGVPDRFSGS | | QMSNLASG |
| | | | PLAPSSKSTSGGTAALGCL | | KGRFTISRDD | | GSGTDFTLRIS | | VPDRFSGSG |
| | | | VKDYFPEPVTVSWNSGAL | | SKNSLYLQM | | RVEAEDVGV | | SGTDFTLRIS |
| | | | TSGVHTFPAVLQSSGLYSL | | NSLKTEDTAV | | YYCAQNLELP | | RVEAEDVG |
| | | | SSVVTVPSSSLGTQTYICN | | YYCTRHYYF | | PTFGQGTKVE | | VYYCAQNL |
| | | | VNHKPSNTKVDKKVEPKS | | DYWGQGTLV | | IKRTVAAPSV | | ELPPTFGQG |
| | | | CDKTHTCPPCPAPELLGGP | | TVSS | | FIFPPSDEQLK | | TKVEIK |
| | | | SVFLFPPKPKDTLMISRTPE | | | | SGTASVVCLL | | |
| | | | VTCVVVDVSHEDPEVKFN | | | | NNFYPREAKV | | |
| | | | WYVDGVEVHNAKTKPRE | | | | QWKVDNALQ | | |
| | | | EQYNSTYRVVSVLTVLHQ | | | | SGNSQESVTE | | |
| | | | DWLNGKEYKCKVSNKAL | | | | QDSKDSTYSL | | |
| | | | PAPIEKTISKAKGQPREPQ | | | | SSTLTLSKAD | | |
| | | | VYTLPPSRDELTKNQVSL | | | | YEKHKVYAC | | |
| | | | WCLVKGFYPSDIAVEWES | | | | EVTHQGLSSP | | |
| | | | NGQPENNYKTTPPVLDSD | | | | VTKSFNRGEC | | |
| | | | GSFFLYSKLTVDKSR WQQ | | | | | | |
| | | | GNVFSCSVMHEALHNHYT | | | | | | |
| | | | QKSLSLSPGK | | | | | | |
| EPI826 | MUC1 | 62 | QVQLVQSGAEVKKPGAS | 63 | QVQLVQSGA | 64 | DIQMTQSPS | 65 | DIQMTQSPS |
| | | | VKVSCKASGYTFSAYWIE | | EVKKPGASV | | SLSASVGDRV | | SLSASVGDR |
| | | | WVRQAPGKGLEWVGEILP | | KVSCKASGYT | | TITCKSSQSLL | | VTITCKSSQ |
| | | | GSGNSRYNEKFKGRVTVT | | FSAYWIEWV | | YSSNQKIYLA | | SLLYSSNQK |
| | | | RDTSTNTAYMELSSLRSED | | RQAPGKGLE | | WYQQKPGKA | | IYLAWYQQ |
| | | | TAVYYCARSYDFAWFAY | | WVGEILPGSG | | PKLLIYWAST | | KPGKAPKLL |
| | | | WGQGTLVTVSSASTKGPS | | NSRYNEKFKG | | RESGVPSRFS | | IYWASTRES |
| | | | VFPLAPSSKSTSGGTAALG | | RVTVTRDTST | | GSGSGTDFTF | | GVPSRFSGS |
| | | | CLVKDYFPEPVTVSWNSG | | NTAYMELSSL | | TISSLQPEDIA | | GSGTDFTFT |
| | | | ALTSGVHTFPAVLQSSGLY | | RSEDTAVYYC | | TYYCQQYYR | | ISSLQPEDIA |
| | | | SLSSVVTVPSSSLGTQTYIC | | ARSYDFAWF | | YPRTFGQGTK | | TYYCQQYY |
| | | | NVNHKPSNTKVDKKVEPK | | AYWGQGTLV | | VEIKRTVAAP | | RYPRTFGQG |
| | | | SCDKTHTCPPCPAPELLGG | | TVSS | | SVFIFPPSDEQ | | TKVEIK |
| | | | PSVFLFPPKPKDTLMISRTP | | | | LKSGTASVVC | | |
| | | | EVTCVVVDVSHEDPEVKF | | | | LLNNFYPREA | | |
| | | | NWYVDGVEVHNAKTKPR | | | | KVQWKVDNA | | |
| | | | EEQYNSTYRVVSVLTVLH | | | | LQSGNSQESV | | |
| | | | QDWLNGKEYKCKVSNKA | | | | TEQDSKDSTY | | |
| | | | LPAPIEKTISKAKGQPREPQ | | | | SLSSTLTLSKA | | |
| | | | VYTLPPSRDELTKNQVSL | | | | DYEKHKVYA | | |
| | | | WCLVKGFYPSDIAVEWES | | | | CEVTHQGLSS | | |
| | | | NGQPENNYKTTPPVLDSD | | | | PVTKSFNRGE | | |
| | | | GSFFLYSKLTVDKSR WQQ | | | | C | | |
| | | | GNVFSCSVMHEALHNHYT | | | | | | |
| | | | QKSLSLSPGK | | | | | | |
| EPI828 | MUC1 | 70 | EVKLVESGGGLVAPGGSL | 71 | EVKLVESGGG | 72 | DVLMTQTPL | 73 | DVLMTQTP |
| | | | KLSCAASGFTFSSYPMSW | | LVAPGGSLKL | | SLPVSLGDQA | | LSLPVSLGD |
| | | | VRQTPEKRLEWVAYINNG | | SCAASGFTFS | | SISCRSSQTIV | | QASISCRSS |
| | | | GGNPYYPDTVKGRFTISRD | | SYPMSWVRQ | | HSNGKIYLEW | | QTIVHSNGK |
| | | | NAKNTLYLQMSSLKSEDT | | TPEKRLEWV | | YLQKPGQSPK | | IYLEWYLQK |
| | | | AIYYCIRQYYGFDYWGQG | | AYINNGGGNP | | LLIYRVSKRFS | | PGQSPKLLI |
| | | | TTLTVSSASTKGPSVFPLA | | YYPDTVKGRF | | GVPDRFSGSG | | YRVSKRFSG |
| | | | PSSKSTSGGTAALGCLVK | | TISRDNAKNT | | SGTDFTLKISR | | VPDRFSGSG |
| | | | DYFPEPVTVSWNSGALTS | | LYLQMSSLKS | | VEAEDLGVY | | SGTDFTLKI |
| | | | GVHTFPAVLQSSGLYSLSS | | EDTAIYYCIR | | YCFQGSHVP | | SRVEAEDLG |
| | | | VVTVPSSSLGTQTYICNVN | | QYYGFDYWG | | WTFGGGTKL | | VYYCFQGS |
| | | | HKPSNTKVDKKVEPKSCD | | QGTTLTVSS | | EIKRTVAAPS | | HVPWTFGG |
| | | | KTHTCPPCPAPELLGGPSV | | | | VFIFPPSDEQL | | GTKLEIK |
| | | | FLFPPKPKDTLMISRTPEVT | | | | KSGTASVVCL | | |
| | | | CVVVDVSHEDPEVKFNW | | | | LNNFYPREAK | | |
| | | | YVDGVEVHNAKTKPREEQ | | | | VQWKVDNAL | | |
| | | | YNSTYRVVSVLTVLHQD | | | | QSGNSQESVT | | |
| | | | WLNGKEYKCKVSNKALP | | | | EQDSKDSTYS | | |
| | | | APIEKTISKAKGQPREPQV | | | | LSSTLTLSKA | | |
| | | | YTLPPSRDELTKNQVSLW | | | | DYEKHKVYA | | |
| | | | CLVKGFYPSDIAVEWESN | | | | CEVTHQGLSS | | |
| | | | GQPENNYKTTPPVLDSDG | | | | PVTKSFNRGE | | |
| | | | SFFLYSKLTVDKSRWQQG | | | | C | | |
| | | | NVFSCSVMHEALHNHYTQ | | | | | | |
| | | | KSLSLSPGK | | | | | | |
| EPI872 | MUC1 | 238 | QMQLVQSEAELKKPGASV | 239 | QMQLVQSEA | 240 | QSVLTQPPS | 241 | QSVLTQPPS |
| | | | KVSCKASGYSFTSHFMHW | | ELKKPGASVK | | VSVAPGKTAR | | VSVAPGKT |
| | | | VRQAPGQGLEWMGWIDP | | VSCKASGYSF | | ITCGGNNIGS | | ARITCGGNN |
| | | | VTGGTKYAQNFQGWVTM | | TSHFMHWVR | | KSVHWYQQK | | IGSKSVHW |
| | | | TRDTSIRTAYLELSRLRSD | | QAPGQGLEW | | PGQAPALVIY | | YQQKPGQA |
| | | | DTAMYYCAREARADRGQ | | MGWIDPVTG | | YGSNRPSGIPE | | PALVIYYGS |
| | | | FDKWGQGTLVTVASASTK | | GTKYAQNFQ | | RFSGSNSGNT | | NRPSGIPERF |
| | | | GPSVFPLAPSSKSTSGGTA | | GWVTMTRDT | | ATLTISRVEA | | SGSNSGNTA |
| | | | ALGCLVKDYFPEPVTVSW | | SIRTAYLELSR | | GDEADYYCQ | | TLTISRVEA |
| | | | NSGALTSGVHTFPAVLQSS | | LRSDDTAMY | | VWDSSSDWV | | GDEADYYC |
| | | | GLYSLSSVVTVPSSSLGTQ | | YCAREARAD | | FGGGTKLTVL | | QVWDSSSD |
| | | | TYICNVNHKPSNTKVDKK | | RGQFDKWGQ | | RTVAAPSVFIF | | WVFGGGTK |
| | | | VEPKSCDKTHTCPPCPAPE | | GTLVTVAS | | PPSDEQLKSG | | LTVL |
| | | | LLGGPSVFLFPPKPKDTLM | | | | TASVVCLLNN | | |
| | | | ISRTPEVTCVVVDVSHEDP | | | | FYPREAKVQ | | |
| | | | EVKFNWYVDGVEVHNAK | | | | WKVDNALQS | | |
| | | | TKPREEQYNSTYRVVSVL | | | | GNSQESVTEQ | | |
| | | | TVLHQDWLNGKEYKCKV | | | | DSKDSTYSLS | | |
| | | | SNKALPAPIEKTISKAKGQ | | | | STLTLSKADY | | |
| | | | PREPQVYTLPPSRDELTKN | | | | EKHKVYACE | | |
| | | | QVSLWCLVKGFYPSDIAV | | | | VTHQGLSSPV | | |
| | | | EWESNGQPENNYKTTPPV | | | | TKSFNRGEC | | |
| | | | LDSDGSFFLYSKLTVDKSR | | | | | | |
| | | | WQQGNVFSCSVMHEALH | | | | | | |
| | | | NHYTQKSLSLSPGK | | | | | | |
| EPI829 | LY75 | 74 | QVQLVESGGGVVQPGRSL | 75 | QVQLVESGG | 76 | EIVLTQSPAT | 77 | EIVLTQSPA |
| | | | RLSCAASGFTFSNYGMYW | | GVVQPGRSLR | | LSLSPGERAT | | TLSLSPGER |
| | | | VRQAPGKGLEWVAVIWY | | LSCAASGFTF | | LSCRASQSVS | | ATLSCRASQ |
| | | | DGSNKYYADSVKGRFTIS | | SNYGMYWVR | | SYLAWYQQK | | SVSSYLAW |
| | | | RDNSKNTLYLQMNSLRAE | | QAPGKGLEW | | PGQAPRLLIY | | YQQKPGQA |
| | | | DTAVYYCARDLWGWYFD | | VAVIWYDGS | | DASNRATGIP | | PRLLIYDAS |
| | | | YWGQGTLVTVSSASTKGP | | NKYYADSVK | | ARFSGSGSGT | | NRATGIPAR |
| | | | SVFPLAPSSKSTSGGTAAL | | GRFTISRDNS | | DFTLTISSLEP | | FSGSGSGTD |
| | | | GCLVKDYFPEPVTVSWNS | | KNTLYLQMN | | EDFAVYYCQ | | FTLTISSLEP |
| | | | GALTSGVHTFPAVLQSSGI | | SLRAEDTAVY | | QRRNWPLTF | | EDFAVYYC |
| | | | YSLSSVVTVPSSSLGTQTYI | | YCARDLWGW | | GGGTKVEIKR | | QQRRNWPL |
| | | | CNVNHKPSNTKVDKKVEP | | YFDYWGQGT | | TVAAPSVFIFP | | TFGGGTKVE |
| | | | KSCDKTHTCPPCPAPELLG | | LVTVSS | | PSDEQLKSGT | | IK |
| | | | GPSVFLFPPKPKDTLMISR | | | | ASVVCLLNNF | | |
| | | | TPEVTCVVVDVSHEDPEV | | | | YPREAKVQW | | |
| | | | KFNWYVDGVEVHNAKTK | | | | KVDNALQSG | | |
| | | | PREEQYNSTYRVVSVLTV | | | | NSQESVTEQD | | |
| | | | LHQDWLNGKEYKCKVSN | | | | SKDSTYSLSS | | |
| | | | KALPAPIEKTISKAKGQPR | | | | TLTLSKADYE | | |
| | | | EPQVYTLPPSRDELTKNQV | | | | KHKVYACEV | | |
| | | | SLWCLVKGFYPSDIAVEW | | | | THQGLSSPVT | | |
| | | | ESNGQPENNYKTTPPVLDS | | | | KSFNRGEC | | |
| | | | DGSFFLYSKLTVDKSRWQ | | | | | | |
| | | | QGNVFSCSVMHEALHNH | | | | | | |
| | | | YTQKSLSLSPGK | | | | | | |
| EPI830 | LY75 | 78 | QVQLVESGGGVVQPGRSL | 79 | QVQLVESGG | 80 | DIQMTQSPS | 81 | DIQMTQSPS |
| | | | RLSCAASGFIFSIYGMHWV | | GVVQPGRSLR | | SLSASVGDRV | | SLSASVGDR |
| | | | RQAPGKGLEWVAVIWYD | | LSCAASGFIFS | | TITCRASQGIS | | VTITCRASQ |
| | | | GSNKYYADSVKGRFTISR | | IYGMHWVRQ | | SWLAWYQQK | | GISSWLAW |
| | | | DNSKNTLYLQMNSLRAED | | APGKGLEWV | | PEKAPKSLIY | | YQQKPEKA |
| | | | TAVYYCARAPHFDYWGQ | | AVIWYDGSN | | AASSLQSGVP | | PKSLIYAAS |
| | | | GTLVTVSSASTKGPSVFPL | | KYYADSVKG | | SRFSGSGSGT | | SLQSGVPSR |
| | | | APSSKSTSGGTAALGCLV | | RFTISRDNSK | | DFTLTISSLQP | | FSGSGSGTD |
| | | | KDYFPEPVTVSWNSGALT | | NTLYLQMNS | | EDFATYYCQ | | FTLTISSLQP |
| | | | SGVHTFPAVLQSSGLYSLS | | LRAEDTAVY | | QYNSYPYTFG | | EDFATYYC |
| | | | SVVTVPSSSLGTQTYICNV | | YCARAPHFD | | QGTKLEIKRT | | QQYNSYPY |
| | | | NHKPSNTKVDKKVEPKSC | | YWGQGTLVT | | VAAPSVFIFPP | | TFGQGTKLE |
| | | | DKTHTCPPCPAPELLGGPS | | VSS | | SDEQLKSGTA | | IK |
| | | | VFLFPPKPKDTLMISRTPE | | | | SVVCLLNNFY | | |
| | | | VTCVVVDVSHEDPEVKFN | | | | PREAKVQWK | | |
| | | | WYVDGVEVHNAKTKPRE | | | | VDNALQSGN | | |
| | | | EQYNSTYRVVSVLTVLHQ | | | | SQESVTEQDS | | |
| | | | DWLNGKEYKCKVSNKAL | | | | KDSTYSLSST | | |
| | | | PAPIEKTISKAKGQPREPQ | | | | LTLSKADYEK | | |
| | | | VYTLPPSRDELTKNQVSL | | | | HKVYACEVT | | |
| | | | WCLVKGFYPSDIAVEWES | | | | HQGLSSPVTK | | |
| | | | NGQPENNYKTTPPVLDSD | | | | SFNRGEC | | |
| | | | GSFFLYSKLTVDKSR WQQ | | | | | | |
| | | | GNVFSCSVMHEALHNHYT | | | | | | |
| | | | QKSLSLSPGK | | | | | | |
| EPI831 | LY75 | 82 | EVQLVESGGGLVKPGGSL | 83 | EVQLVESGGG | 84 | DVQMTQSPS | 85 | DVQMTQSP |
| | | | RLSCAASGFTYSNAWMS | | LVKPGGSLRL | | SLSASVGDRV | | SSLSASVGD |
| | | | WVRQAPGKGLEWVGRIK | | SCAASGFTYS | | TITCRASQSIS | | RVTITCRAS |
| | | | SKTDGGTTDYAAPVQGRF | | NAWMSWVR | | DYLSWYQQR | | QSISDYLSW |
| | | | TISRDDSKNTLYLQMNSL | | QAPGKGLEW | | PGKAPNLLIY | | YQQRPGKA |
| | | | KTEDTAVYYCTIFGVVSFD | | VGRIKSKTDG | | AASNLKTGVP | | PNLLIYAAS |
| | | | YWGQGTLVTVSSASTKGP | | GTTDYAAPV | | SRFSGSGSGT | | NLKTGVPSR |
| | | | SVFPLAPSSKSTSGGTAAL | | QGRFTISRDD | | DFTLTISTLQP | | FSGSGSGTD |
| | | | GCLVKDYFPEPVTVSWNS | | SKNTLYLQM | | EDFATYYCQ | | FTLTISTLQP |
| | | | GALTSGVHTFPAVLQSSGL | | NSLKTEDTAV | | QSYRSPWTFG | | EDFATYYC |
| | | | YSLSSVVTVPSSSLGTQTYI | | YYCTIFGVVS | | QGTKVEIKRT | | QQSYRSPW |
| | | | CNVNHKPSNTKVDKKVEP | | FDYWGQGTL | | VAAPSVFIFPP | | TFGQGTKVE |
| | | | KSCDKTHTCPPCPAPELLG | | VTVSS | | SDEQLKSGTA | | IK |
| | | | GPSVFLFPPKPKDTLMISR | | | | SVVCLLNNFY | | |
| | | | TPEVTCVVVDVSHEDPEV | | | | PREAKVQWK | | |
| | | | KFNWYVDGVEVHNAKTK | | | | VDNALQSGN | | |
| | | | PREEQYNSTYRVVSVLTV | | | | SQESVTEQDS | | |
| | | | LHQDWLNGKEYKCKVSN | | | | KDSTYSLSST | | |
| | | | KALPAPIEKTISKAKGQPR | | | | LTLSKADYEK | | |
| | | | EPQVYTLPPSRDELTKNQV | | | | HKVYACEVT | | |
| | | | SLWCLVKGFYPSDIAVEW | | | | HQGLSSPVTK | | |
| | | | ESNGQPENNYKTTPPVLDS | | | | SFNRGEC | | |
| | | | DGSFFLYSKLTVDKSRWQ | | | | | | |
| | | | QGNVFSCSVMHEALHNH | | | | | | |
| | | | YTQKSLSLSPGK | | | | | | |

| | | | | | | | | | |
|---|---|---|---|---|---|---|---|---|---|
| EPI834 | CD276 | 94 | QVQLQQSGAELVKPGASV | 95 | QVQLQQSGA | 96 | DIVMTQSPA | 97 | DIVMTQSPA |
| | | | KLSCKASGYTFTNYDINW | | ELVKPGASVK | | TLSVTPGDRV | | TLSVTPGDR |
| | | | VRQRPEQGLEWIGWIFPG | | LSCKASGYTF | | SLSCRASQSIS | | VSLSCRASQ |
| | | | DGSTQYNEKFKGKATLTT | | TNYDINWVR | | DYLHWYQQK | | SISDYLHWY |
| | | | DTSSSTAYMQLSRLTSEDS | | QRPEQGLEWI | | SHESPRLLIKY | | QQKSHESPR |
| | | | AVYFCARQTTATWFAYW | | GWIFPGDGST | | ASQSISGIPSR | | LLIKYASQSI |
| | | | GQGTLVTVSAASTKGPSV | | QYNEKFKGK | | FSGSGSGSDF | | SGIPSRFSGS |
| | | | FPLAPSSKSTSGGTAALGC | | ATLTTDTSSS | | TLSINSVEPED | | GSGSDFTLSI |
| | | | LVKDYFPEPVTVSWNSGA | | TAYMQLSRLT | | VGVYYCQNG | | NSVEPEDVG |
| | | | LTSGVHTFPAVLQSSGLYS | | SEDSAVYFCA | | HSFPLTFGAG | | VYYCQNGH |
| | | | LSSVVTVPSSSLGTQTYIC | | RQTTATWFA | | TKLELKRTVA | | SFPLTFGAG |
| | | | NVNHKPSNTKVDKKVEPK | | YWGQGTLVT | | APSVFIFPPSD | | TKLELK |
| | | | SCDKTHTCPPCPAPELLGG | | VSA | | EQLKSGTASV | | |
| | | | PSVFLFPPKPKDTLMISRTP | | | | VCLLNNFYPR | | |
| | | | EVTCVVVDVSHEDPEVKF | | | | EAKVQWKVD | | |
| | | | NWYVDGVEVHNAKTKPR | | | | NALQSGNSQE | | |
| | | | EEQYNSTYRVVSVLTVLH | | | | SVTEQDSKDS | | |
| | | | QDWLNGKEYKCKVSNKA | | | | TYSLSSTLTLS | | |
| | | | LPAPIEKTISKAKGQPREPQ | | | | KADYEKHKV | | |
| | | | VYTLPPSRDELTKNQVSL | | | | YACEVTHQG | | |
| | | | WCLVKGFYPSDIAVEWES | | | | LSSPVTKSFN | | |
| | | | NGQPENNYKTTPPVLDSD | | | | RGEC | | |
| | | | GSFFLYSKLTVDKSRWQQ | | | | | | |
| | | | GNVFSCSVMHEALHNHYT | | | | | | |
| | | | QKSLSLSPGK | | | | | | |
| EPI835 | CD276 | 98 | EVQLQESGPGLVKPSETLS | 99 | EVQLQESGPG | 100 | DIQMTQSPS | 101 | DIQMTQSPS |
| | | | LTCAVTGYSITSGYSWHW | | LVKPSETLSL | | SLSASVGDRV | | SLSASVGDR |
| | | | IRQFPGNGLEWMGYIHSS | | TCAVTGYSIT | | TITCKASQNV | | VTITCKASQ |
| | | | GSTNYNPSLKSRISISRDTS | | SGYSWHWIR | | GFNVAWYQQ | | NVGFNVAW |
| | | | KNQFFLKLSSVTAADTAV | | QFPGNGLEW | | KPGKSPKALI | | YQQKPGKS |
| | | | YYCAGYDDYFEYWGQGT | | MGYIHSSGST | | YSASYRYSGV | | PKALIYSAS |
| | | | TVTVSSASTKGPSVFPLAP | | NYNPSLKSRIS | | PSRFSGSGSG | | YRYSGVPSR |
| | | | SSKSTSGGTAALGCLVKD | | ISRDTSKNQFF | | TDFTLTISSLQ | | FSGSGSGTD |
| | | | YFPEPVTVSWNSGALTSG | | LKLSSVTAAD | | PEDFAEYFCQ | | FTLTISSLQP |
| | | | VHTFPAVLQSSGLYSLSSV | | TAVYYCAGY | | QYNWYPFTF | | EDFAEYFCQ |
| | | | VTVPSSSLGTQTYICNVNH | | DDYFEYWGQ | | GQGTKLEIKR | | QYNWYPFT |
| | | | KPSNTKVDKKVEPKSCDK | | GTTVTVSS | | TVAAPSVFIFP | | FGQGTKLEI |
| | | | THTCPPCPAPELLGGPSVF | | | | PSDEQLKSGT | | K |
| | | | LFPPKPKDTLMISRTPEVT | | | | ASVVCLLNNF | | |
| | | | CVVVDVSHEDPEVKFNW | | | | YPREAKVQW | | |
| | | | YVDGVEVHNAKTKPREEQ | | | | KVDNALQSG | | |
| | | | YNSTYRVVSVLTVLHQD | | | | NSQESVTEQD | | |
| | | | WLNGKEYKCKVSNKALP | | | | SKDSTYSLSS | | |
| | | | APIEKTISKAKGQPREPQV | | | | TLTLSKADYE | | |
| | | | YTLPPSRDELTKNQVSLW | | | | KHKVYACEV | | |
| | | | CLVKGFYPSDIAVEWESN | | | | THQGLSSPVT | | |
| | | | GQPENNYKTTPPVLDSDG | | | | KSFNRGEC | | |
| | | | SFFLYSKLTVDKSRWQQG | | | | | | |
| | | | NVFSCSVMHEALHNHYTQ | | | | | | |
| | | | KSLSLSPGK | | | | | | |
| EPI836 | CD276 | 102 | QVQLVQSGAEVKKPGSSV | 103 | QVQLVQSGA | 104 | EIVLTQSPAT | 105 | EIVLTQSPA |
| | | | KVSCKASGYTFTNYVMH | | EVKKPGSSVK | | LSLSPGERAT | | TLSLSPGER |
| | | | WVRQAPGQGLEWMGYIN | | VSCKASGYTF | | LSCRASSRLIY | | ATLSCRASS |
| | | | PYNDDVKYNEKFKGRVTI | | TNYVMHWVR | | MHWYQQKPG | | RLIYMHWY |
| | | | TADESTSTAYMELSSLRSE | | QAPGQGLEW | | QAPRPLIYAT | | QQKPGQAP |
| | | | DTAVYYCARWGYYGSPL | | MGYINPYND | | SNLASGIPARF | | RPLIYATSN |
| | | | YYFDYWGQGTLVTVSSAS | | DVKYNEKFK | | SGSGSGTDFT | | LASGIPARF |
| | | | TKGPSVFPLAPSSKSTSGG | | GRVTITADES | | LTISSLEPEDF | | SGSGSGTDF |
| | | | TAALGCLVKDYFPEPVTV | | TSTAYMELSS | | AVYYCQQWN | | TLTISSLEPE |
| | | | SWNSGALTSGVHTFPAVL | | LRSEDTAVYY | | SNPPTFGQGT | | DFAVYYCQ |
| | | | QSSGLYSLSSVVTVPSSSL | | CARWGYYGS | | KVEIKRTVAA | | QWNSNPPTF |
| | | | GTQTYICNVNHKPSNTKV | | PLYYFDYWG | | PSVFIFPPSDE | | GQGTKVEIK |
| | | | DKKVEPKSCDKTHTCPPC | | QGTLVTVSS | | QLKSGTASVV | | |
| | | | PAPELLGGPSVFLFPPKPK | | | | CLLNNFYPRE | | |
| | | | DTLMISRTPEVTCVVVDVS | | | | AKVQWKVDN | | |
| | | | HEDPEVKFNWYVDGVEV | | | | ALQSGNSQES | | |
| | | | HNAKTKPREEQYNSTYRV | | | | VTEQDSKDST | | |
| | | | VSVLTVLHQDWLNGKEY | | | | YSLSSTLTLSK | | |
| | | | KCKVSNKALPAPIEKTISK | | | | ADYEKHKVY | | |
| | | | AKGQPREPQVYTLPPSRDE | | | | ACEVTHQGLS | | |
| | | | LTKNQVSLWCLVKGFYPS | | | | SPVTKSFNRG | | |
| | | | DIAVEWESNGQPENNYKT | | | | EC | | |
| | | | TPPVLDSDGSFFLYSKLTV | | | | | | |
| | | | DKSRWQQGNVFSCSVMH | | | | | | |
| | | | EALHNHYTQKSLSLSPGK | | | | | | |
| EPI1003 | CD276 | 258 | EVQLVESGGGLVQPGGSL | 259 | EVQLVESGGG | 260 | DIQLTQSPSF | 261 | DIQLTQSPSF |
| | | | RLSCAASGFTFSSFGMHW | | LVQPGGSLRL | | LSASVGDRVT | | LSASVGDR |
| | | | VRQAPGKGLEWVAYISSD | | SCAASGFTFS | | ITCKASQNVD | | VTITCKASQ |
| | | | SSAIYYADTVKGRFTISRD | | SFGMHWVRQ | | TNVAWYQQK | | NVDTNVAW |
| | | | NAKNSLYLQMNSLRDEDT | | APGKGLEWV | | PGKAPKALIY | | YQQKPGKA |
| | | | AVYYCGRGRENIYYGSRL | | AYISSDSSAIY | | SASYRYSGVP | | PKALIYSAS |
| | | | DYWGQGTTVTVSSASTKG | | YADTVKGRF | | SRFSGSGSGT | | YRYSGVPSR |
| | | | PSVFPLAPSSKSTSGGTAA | | TISRDNAKNS | | DFTLTISSLQP | | FSGSGSGTD |
| | | | LGCLVKDYFPEPVTVSWN | | LYLQMNSLR | | EDFATYYCQ | | FTLTISSLQP |
| | | | SGALTSGVHTFPAVLQSSG | | DEDTAVYYC | | QYNNYPFTFG | | EDFATYYC |
| | | | LYSLSSVVTVPSSSLGTQT | | GRGRENIYYG | | QGTKLEIKRT | | QQYNNYPF |
| | | | YICNVNHKPSNTKVDKRV | | SRLDYWGQG | | VAAPSVFIFPP | | TFGQGTKLE |
| | | | EPKSCDKTHTCPPCPAPEL | | TTVTVSS | | SDEQLKSGTA | | IK |
| | | | LGGPSVFLFPPKPKDTLMI | | | | SVVCLLNNFY | | |
| | | | SRTPEVTCVVVDVSHEDP | | | | PREAKVQWK | | |
| | | | EVKFNWYVDGVEVHNAK | | | | VDNALQSGN | | |
| | | | TKPREEQYNSTYRVVSVL | | | | SQESVTEQDS | | |
| | | | TVLHQDWLNGKEYKCKV | | | | KDSTYSLSST | | |
| | | | SNKALPAPIEKTISKAKGQ | | | | LTLSKADYEK | | |
| | | | PREPQVYTLPPSRDELTKN | | | | HKVYACEVT | | |
| | | | QVSLWCLVKGFYPSDIAV | | | | HQGLSSPVTK | | |
| | | | EWESNGQPENNYKTTPPV | | | | SFNRGEC | | |
| | | | LDSDGSFFLYSKLTVDKSR | | | | | | |
| | | | WQQGNVFSCSVMHEALH | | | | | | |
| | | | NHYTQKSLSLSPGKGGSH | | | | | | |
| | | | HHHHH | | | | | | |
| EPI837 | MST1R | 106 | EVQLVESGGGLVQPGGSL | 107 | EVQLVESGGG | 108 | EIVLTQSPAT | 109 | EIVLTQSPA |
| | | | RLSCAASGFTFSSYLMTW | | LVQPGGSLRL | | LSLSPGERAT | | TLSLSPGER |
| | | | VRQAPGKGLEWVANIKQ | | SCAASGFTFS | | LSCRASQSVS | | ATLSCRASQ |
| | | | DGSEKYYVDSVKGRFTISR | | SYLMTWVRQ | | RYLAWYQQK | | SVSRYLAW |
| | | | DNAKNSLNLQMNSLRAED | | APGKGLEWV | | PGQAPRLLIY | | YQQKPGQA |
| | | | TAVYYCTRDGYSSGRHYG | | ANIKQDGSEK | | DASNRATGIP | | PRLLIYDAS |
| | | | MDVWGQGTTVIVSSASTK | | YYVDSVKGR | | ARFSGSGSGT | | NRATGIPAR |
| | | | GPSVFPLAPSSKSTSGGTA | | FTISRDNAKN | | DFTLTISSLEP | | FSGSGSGTD |
| | | | ALGCLVKDYFPEPVTVSW | | SLNLQMNSLR | | EDFAVYYCQ | | FTLTISSLEP |
| | | | NSGALTSGVHTFPAVLQSS | | AEDTAVYYC | | QRSNWPRTFG | | EDFAVYYC |
| | | | GLYSLSSVVTVPSSSLGTQ | | TRDGYSSGRH | | QGTKVEIKRT | | QQRSNWPR |
| | | | TYICNVNHKPSNTKVDKK | | YGMDVWGQ | | VAAPSVFIFPP | | TFGQGTKVE |
| | | | VEPKSCDKTHTCPPCPAPE | | GTTVIVSS | | SDEQLKSGTA | | IK |
| | | | LLGGPSVFLFPPKPKDTLM | | | | SVVCLLNNFY | | |
| | | | ISRTPEVTCVVVDVSHEDP | | | | PREAKVQWK | | |
| | | | EVKFNWYVDGVEVHNAK | | | | VDNALQSGN | | |
| | | | TKPREEQYNSTYRVVSVL | | | | SQESVTEQDS | | |
| | | | TVLHQDWLNGKEYKCKV | | | | KDSTYSLSST | | |
| | | | SNKALPAPIEKTISKAKGQ | | | | LTLSKADYEK | | |
| | | | PREPQVYTLPPSRDELTKN | | | | HKVYACEVT | | |
| | | | QVSLWCLVKGFYPSDIAV | | | | HQGLSSPVTK | | |
| | | | EWESNGQPENNYKTTPPV | | | | SFNRGEC | | |
| | | | LDSDGSFFLYSKLTVDKSR | | | | | | |
| | | | WQQGNVFSCSVMHEALH | | | | | | |
| | | | NHYTQKSLSLSPGK | | | | | | |
| EPI838 | MST1R | 110 | EVQLVESGGGLVQPGGSL | 111 | EVQLVESGGG | 112 | DIQLTQSQS | 113 | DIQLTQSQS |
| | | | RLSCAASGFTFSRHWMSW | | LVQPGGSLRL | | FVSTSVGDRV | | FVSTSVGDR |
| | | | VRQAPGKGLEWVSEINPD | | SCAASGFTFS | | TVTCRASQNV | | VTVTCRAS |
| | | | SRTINYAPSVKGRFTISRD | | RHWMSWVR | | GSSLVWYQQ | | QNVGSSLV |
| | | | NAKNSLYLQMNSLRAEDT | | QAPGKGLEW | | KPGKSPKTLI | | WYQQKPGK |
| | | | AVYYCARRVRIHYYGAM | | VSEINPDSRTI | | YSASFLYSGV | | SPKTLIYSAS |
| | | | DSWGQGTTVTVSSASTKG | | NYAPSVKGRF | | PSRFSGSGSG | | FLYSGVPSR |
| | | | PSVFPLAPSSKSTSGGTAA | | TISRDNAKNS | | TEFTLTISSVQ | | FSGSGSGTE |
| | | | LGCLVKDYFPEPVTVSWN | | LYLQMNSLR | | PEDFADYFCQ | | FTLTISSVQP |
| | | | SGALTSGVHTFPAVLQSSG | | AEDTAVYYC | | QYNNYPLTFG | | EDFADYFC |
| | | | LYSLSSVVTVPSSSLGTQT | | ARRVRIHYYG | | GGTKVEIKRT | | QQYNNYPL |
| | | | YICNVNHKPSNTKVDKKV | | AMDSWGQGT | | VAAPSVFIFPP | | TFGGGTKVE |
| | | | EPKSCDKTHTCPPCPAPEL | | TVTVSS | | SDEQLKSGTA | | IK |
| | | | LGGPSVFLFPPKPKDTLMI | | | | SVVCLLNNFY | | |
| | | | SRTPEVTCVVVDVSHEDP | | | | PREAKVQWK | | |
| | | | EVKFNWYVDGVEVHNAK | | | | VDNALQSGN | | |
| | | | TKPREEQYNSTYRVVSVL | | | | SQESVTEQDS | | |
| | | | TVLHQDWLNGKEYKCKV | | | | KDSTYSLSST | | |
| | | | SNKALPAPIEKTISKAKGQ | | | | LTLSKADYEK | | |
| | | | PREPQVYTLPPSRDELTKN | | | | HKVYACEVT | | |
| | | | QVSLWCLVKGFYPSDIAV | | | | HQGLSSPVTK | | |
| | | | EWESNGQPENNYKTTPPV | | | | SFNRGEC | | |
| | | | LDSDGSFFLYSKLTVDKSR | | | | | | |
| | | | WQQGNVFSCSVMHEALH | | | | | | |
| | | | NHYTQKSLSLSPGK | | | | | | |
| EPI839 | MST1R | 114 | EVQLQQSGAELVKPGASV | 115 | EVQLQQSGAE | 116 | DIQMNQSPS | 117 | DIQMNQSPS |
| | | | KLSCTTSGFNIIDTYIHWV | | LVKPGASVKL | | SLSASLGDTIT | | SLSASLGDT |
| | | | NQKPDQGLEWIGRIDPAD | | SCTTSGFNIID | | ITCHASQNIN | | ITITCHASQN |
| | | | GNRKSDPKFQVKATITVD | | TYIHWVNQK | | VWLNWYQQ | | INVWLNWY |
| | | | TSSNTAYLQLSSLTSGDTA | | PDQGLEWIGR | | KPGNIPKLLIY | | QQKPGNIPK |
| | | | VYYCARGYGNLNAMDSW | | IDPADGNRKS | | KASNLHTGVP | | LLIYKASNL |
| | | | GQGTSVTVSSASTKGPSVF | | DPKFQVKATI | | SRFSGSGSGT | | HTGVPSRFS |
| | | | PLAPSSKSTSGGTAALGCL | | TVDTSSNTAY | | GFTLTISSLQP | | GSGSGTGFT |
| | | | VKDYFPEPVTVSWNSGAL | | LQLSSLTSGD | | EDIATYYCQQ | | LTISSLQPED |
| | | | TSGVHTFPAVLQSSGLYSL | | TAVYYCARG | | GQSYPLTFGG | | IATYYCQQG |
| | | | SSVVTVPSSSLGTQTYICN | | YGNLNAMDS | | GTKLEIKRTV | | QSYPLTFGG |
| | | | VNHKPSNTKVDKKVEPKS | | WGQGTSVTV | | AAPSVFIFPPS | | GTKLEIK |
| | | | CDKTHTCPPCPAPELLGGP | | SS | | DEQLKSGTAS | | |
| | | | SVFLFPPKPKDTLMISRTPE | | | | VVCLLNNFYP | | |
| | | | VTCVVVDVSHEDPEVKFN | | | | REAKVQWKV | | |
| | | | WYVDGVEVHNAKTKPRE | | | | DNALQSGNS | | |
| | | | EQYNSTYRVVSVLTVLHQ | | | | QESVTEQDSK | | |
| | | | DWLNGKEYKCKVSNKAL | | | | DSTYSLSSTLT | | |
| | | | PAPIEKTISKAKGQPREPQ | | | | LSKADYEKH | | |
| | | | VYTLPPSRDELTKNQVSL | | | | KVYACEVTH | | |
| | | | WCLVKGFYPSDIAVEWES | | | | QGLSSPVTKS | | |
| | | | NGQPENNYKTTPPVLDSD | | | | FNRGEC | | |
| | | | GSFFLYSKLTVDKSRWQQ | | | | | | |
| | | | GNVFSCSVMHEALHNHYT | | | | | | |
| | | | QKSLSLSPGK | | | | | | |
| EPI840 | MST1R | 118 | QVQLVQSGAEVKKPGAT | 119 | QVQLVQSGA | 120 | EIVMTQSPG | 121 | EIVMTQSPG |
| | | | VKISCKVSGYTFTDYHMD | | EVKKPGATV | | TLSLSPGERA | | TLSLSPGER |
| | | | WVQQAPGKGLEWMGDIN | | KISCKVSGYT | | TLSCKSSQSL | | ATLSCKSSQ |
| | | | PNNGGAIYNQKFKGRVTIT | | FTDYHMDWV | | LFSGNQKNYL | | SLLFSGNQK |
| | | | ADTSTDTAYMELSSLRSE | | QQAPGKGLE | | AWYQQKPGQ | | NYLAWYQQ |
| | | | DTAVYYCARSHYDYAGG | | WMGDINPNN | | APRLLIYWAS | | KPGQAPRLL |
| | | | AWFAYWGQGTLVTVSRA | | GGAIYNQKFK | | TRASGIPDRFS | | IYWASTRAS |
| | | | STKGPSVFPLAPSSKSTSG | | GRVTITADTS | | GSGSGTDFTL | | GIPDRFSGS |
| | | | GTAALGCLVKDYFPEPVT | | TDTAYMELSS | | TISRLEPEDFA | | GSGTDFTLT |
| | | | VSWNSGALTSGVHTFPAV | | LRSEDTAVYY | | VYYCQQYYS | | ISRLEPEDFA |
| | | | LQSSGLYSLSSVVTVPSSS | | CARSHYDYA | | FPRTFGQGTK | | VYYCQQYY |
| | | | LGTQTYICNVNHKPSNTK | | GGAWFAYW | | LEIKRTVAAP | | SFPRTFGQG |
| | | | VDKKVEPKSCDKTHTCPP | | GQGTLVTVSR | | SVFIFPPSDEQ | | TKLEIK |
| | | | CPAPELLGGPSVFLFPPKP | | | | LKSGTASVVC | | |
| | | | KDTLMISRTPEVTCVVVD | | | | LLNNFYPREA | | |
| | | | VSHEDPEVKFNWYVDGV | | | | KVQWKVDNA | | |
| | | | EVHNAKTKPREEQYNSTY | | | | LQSGNSQESV | | |
| | | | RVVSVLTVLHQDWLNGK | | | | TEQDSKDSTY | | |
| | | | EYKCKVSNKALPAPIEKTI | | | | SLSSTLTLSKA | | |
| | | | SKAKGQPREPQVYTLPPSR | | | | DYEKHKVYA | | |
| | | | DELTKNQVSLWCLVKGFY | | | | CEVTHQGLSS | | |
| | | | PSDIAVEWESNGQPENNY | | | | PVTKSFNRGE | | |
| | | | KTTPPVLDSDGSFFLYSKL | | | | C | | |
| | | | TVDKSRWQQGNVFSCSV | | | | | | |
| | | | MHEALHNHYTQKSLSLSP | | | | | | |
| | | | GK | | | | | | |
| EPI841 | MSLN | 122 | QVELVQSGAEVKKPGESL | 123 | QVELVQSGAE | 124 | DIALTQPAS | 125 | DIALTQPAS |
| | | | KISCKGSGYSFTSYWIGW | | VKKPGESLKI | | VSGSPGQSITI | | VSGSPGQSI |
| | | | VRQAPGKGLEWMGIIDPG | | SCKGSGYSFT | | SCTGTSSDIG | | TISCTGTSSD |
| | | | DSRTRYSPSFQGQVTISAD | | SYWIGWVRQ | | GYNSVSWYQ | | IGGYNSVS |
| | | | KSISTAYLQWSSLKASDTA | | APGKGLEWM | | QHPGKAPKL | | WYQQHPGK |
| | | | MYYCARGQLYGGTYMDG | | GIIDPGDSRTR | | MIYGVNNRPS | | APKLMIYG |
| | | | WGQGTLVTVSSASTKGPS | | YSPSFQGQVT | | GVSNRFSGSK | | VNNRPSGVS |
| | | | VFPLAPSSKSTSGGTAALG | | ISADKSISTAY | | SGNTASLTISG | | NRFSGSKSG |
| | | | CLVKDYFPEPVTVSWNSG | | LQWSSLKASD | | LQAEDEADY | | NTASLTISG |
| | | | ALTSGVHTFPAVLQSSGLY | | TAMYYCARG | | YCSSYDIESA | | LQAEDEAD |
| | | | SLSSVVTVPSSSLGTQTYIC | | QLYGGTYMD | | TPVFGGGTKL | | YYCSSYDIE |
| | | | NVNHKPSNTKVDKKVEPK | | GWGQGTLVT | | TVLRTVAAPS | | SATPVFGGG |
| | | | SCDKTHTCPPCPAPELLGG | | VSS | | VFIFPPSDEQL | | TKLTVL |
| | | | PSVFLFPPKPKDTLMISRTP | | | | KSGTASVVCL | | |
| | | | EVTCVVVDVSHEDPEVKF | | | | LNNFYPREAK | | |
| | | | NWYVDGVEVHNAKTKPR | | | | VQWKVDNAL | | |
| | | | EEQYNSTYRVVSVLTVLH | | | | QSGNSQESVT | | |
| | | | QDWLNGKEYKCKVSNKA | | | | EQDSKDSTYS | | |
| | | | LPAPIEKTISKAKGQPREPQ | | | | LSSTLTLSKA | | |
| | | | VYTLPPSRDELTKNQVSL | | | | DYEKHKVYA | | |
| | | | WCLVKGFYPSDIAVEWES | | | | CEVTHQGLSS | | |
| | | | NGQPENNYKTTPPVLDSD | | | | PVTKSFNRGE | | |
| | | | GSFFLYSKLTVDKSRWQQ | | | | C | | |
| | | | GNVFSCSVMHEALHNHYT | | | | | | |
| | | | QKSLSLSPGK | | | | | | |
| EPI842 | MSLN | 126 | QVQLQQSGPELEKPGASV | 127 | QVQLQQSGPE | 128 | DIELTQSPAI | 129 | DIELTQSPAI |
| | | | KISCKASGYSFTGYTMNW | | LEKPGASVKI | | MSASPGEKVT | | MSASPGEK |
| | | | VKQSHGKSLEWIGLITPYN | | SCKASGYSFT | | MTCSASSSVS | | VTMTCSASS |
| | | | GASSYNQKFRGKATLTVD | | GYTMNWVK | | YMHWYQQKS | | SVSYMHWY |
| | | | KSSSTAYMDLLSLTSEDSA | | QSHGKSLEWI | | GTSPKRWIYD | | QQKSGTSPK |
| | | | VYFCARGGYDGRGFDYW | | GLITPYNGAS | | TSKLASGVPG | | RWIYDTSKL |
| | | | GSGTPVTVSSASTKGPSVF | | SYNQKFRGK | | RFSGSGSGNS | | ASGVPGRFS |
| | | | PLAPSSKSTSGGTAALGCL | | ATLTVDKSSS | | YSLTISSVEAE | | GSGSGNSYS |
| | | | VKDYFPEPVTVSWNSGAL | | TAYMDLLSLT | | DDATYYCQQ | | LTISSVEAE |
| | | | TSGVHTFPAVLQSSGLYSL | | SEDSAVYFCA | | WSKHPLTFGS | | DDATYYCQ |
| | | | SSVVTVPSSSLGTQTYICN | | RGGYDGRGF | | GTKVEIKRTV | | QWSKHPLT |
| | | | VNHKPSNTKVDKKVEPKS | | DYWGSGTPV | | AAPSVFIFPPS | | FGSGTKVEI |
| | | | CDKTHTCPPCPAPELLGGP | | TVSS | | DEQLKSGTAS | | K |
| | | | SVFLFPPKPKDTLMISRTPE | | | | VVCLLNNFYP | | |
| | | | VTCVVVDVSHEDPEVKFN | | | | REAKVQWKV | | |
| | | | WYVDGVEVHNAKTKPRE | | | | DNALQSGNS | | |
| | | | EQYNSTYRVVSVLTVLHQ | | | | QESVTEQDSK | | |
| | | | DWLNGKEYKCKVSNKAL | | | | DSTYSLSSTLT | | |
| | | | PAPIEKTISKAKGQPREPQ | | | | LSKADYEKH | | |
| | | | VYTLPPSRDELTKNQVSL | | | | KVYACEVTH | | |
| | | | WCLVKGFYPSDIAVEWES | | | | QGLSSPVTKS | | |
| | | | INGQPENNYKTTPPVLDSD | | | | FNRGEC | | |
| | | | GSFFLYSKLTVDKSRWQQ | | | | | | |
| | | | GNVFSCSVMHEALHNHYT | | | | | | |
| | | | QKSLSLSPGK | | | | | | |
| EPI843 | MSLN | 130 | QVHLVESGGGVVQPGRSL | 131 | QVHLVESGG | 132 | EIVLTQSPAT | 133 | EIVLTQSPA |
| | | | RLSCVASGITFRIYGMHW | | GVVQPGRSLR | | LSLSPGERAT | | TLSLSPGER |
| | | | VRQAPGKGLEWVAVLWY | | LSCVASGITFR | | LSCRASQSVS | | ATLSCRASQ |
| | | | DGSHEYYADSVKGRFTISR | | IYGMHWVRQ | | SYLAWYQQK | | SVSSYLAW |
| | | | DNSKNTLYLQMNSLRAED | | APGKGLEWV | | PGQAPRLLIY | | YQQKPGQA |
| | | | TAIYYCARDGDYYDSGSP | | AVLWYDGSH | | DASNRATGIP | | PRLLIYDAS |
| | | | LDYWGQGTLVTVSSASTK | | EYYADSVKG | | ARFSGSGSGT | | NRATGIPAR |
| | | | GPSVFPLAPSSKSTSGGTA | | RFTISRDNSK | | DFTLTISSLEP | | FSGSGSGTD |
| | | | ALGCLVKDYFPEPVTVSW | | NTLYLQMNS | | EDFAVYYCQ | | FTLTISSLEP |
| | | | NSGALTSGVHTFPAVLQSS | | LRAEDTAIYY | | QRSNWPLTFG | | EDFAVYYC |
| | | | GLYSLSSVVTVPSSSLGTQ | | CARDGDYYD | | GGTKVEIKRT | | QQRSNWPL |
| | | | TYICNVNHKPSNTKVDKK | | SGSPLDYWG | | VAAPSVFIFPP | | TFGGGTKVE |
| | | | VEPKSCDKTHTCPPCPAPE | | QGTLVTVSS | | SDEQLKSGTA | | K |
| | | | LLGGPSVFLFPPKPKDTLM | | | | SVVCLLNNFY | | |
| | | | ISRTPEVTCVVVDVSHEDP | | | | PREAKVQWK | | |
| | | | EVKFNWYVDGVEVHNAK | | | | VDNALQSGN | | |
| | | | TKPREEQYNSTYRVVSVL | | | | SQESVTEQDS | | |
| | | | TVLHQDWLNGKEYKCKV | | | | KDSTYSLSST | | |
| | | | SNKALPAPIEKTISKAKGQ | | | | LTLSKADYEK | | |
| | | | PREPQVYTLPPSRDELTKN | | | | HKVYACEVT | | |
| | | | QVSLWCLVKGFYPSDIAV | | | | HQGLSSPVTK | | |
| | | | EWESNGQPENNYKTTPPV | | | | SFNRGEC | | |
| | | | LDSDGSFFLYSKLTVDKSR | | | | | | |
| | | | WQQGNVFSCSVMHEALH | | | | | | |
| | | | NHYTQKSLSLSPGK | | | | | | |
| EPI844 | MSLN | 134 | EVQLVQSGAEVKKPGASV | 135 | EVQLVQSGAE | 136 | SYELTQPPS | 137 | SYELTQPPS |
| | | | KVSCKASGDTFKRYYVH | | VKKPGASVK | | VSVSPGQTAS | | VSVSPGQTA |
| | | | WVRQAPGQGLEWMGIINP | | VSCKASGDTF | | ITCSGDKLGD | | SITCSGDKL |
| | | | SGVSTTYAQKFQGRVTMT | | KRYYVHWVR | | KYASWYQQK | | GDKYASWY |
| | | | RDTSTSTVYMELSSLRSED | | QAPGQGLEW | | PGQSPVLVIY | | QQKPGQSP |
| | | | TAVYYCAEVRGSGFNYFG | | MGIINPSGVST | | QDNRRPSGIP | | VLVIYQDNR |
| | | | MDVWGQGTLVTVSSAST | | TYAQKFQGR | | ERFSGSNSGN | | RPSGIPERFS |
| | | | KGPSVFPLAPSSKSTSGGT | | VTMTRDTSTS | | TATLTISGTQ | | GSNSGNTAT |
| | | | AALGCLVKDYFPEPVTVS | | TVYMELSSLR | | AMDEADYYC | | LTISGTQAM |
| | | | WNSGALTSGVHTFPAVLQ | | SEDTAVYYC | | QAWDSDTYV | | DEADYYCQ |
| | | | SSGLYSLSSVVTVPSSSLG | | AEVRGSGFNY | | FGTGTKVTVL | | AWDSDTYV |
| | | | TQTYICNVNHKPSNTKVD | | FGMDVWGQG | | RTVAAPSVFIF | | FGTGTKVTV |
| | | | KKVEPKSCDKTHTCPPCP | | TLVTVSS | | PPSDEQLKSG | | L |
| | | | APELLGGPSVFLFPPKPKD | | | | TASVVCLLNN | | |
| | | | TLMISRTPEVTCVVVDVSH | | | | FYPREAKVQ | | |
| | | | EDPEVKFNWYVDGVEVH | | | | WKVDNALQS | | |
| | | | NAKTKPREEQYNSTYRVV | | | | GNSQESVTEQ | | |
| | | | SVLTVLHQDWLNGKEYK | | | | DSKDSTYSLS | | |
| | | | CKVSNKALPAPIEKTISKA | | | | STLTLSKADY | | |
| | | | KGQPREPQVYTLPPSRDEL | | | | EKHKVYACE | | |
| | | | TKNQVSLWCLVKGFYPSD | | | | VTHQGLSSPV | | |
| | | | IAVEWESNGQPENNYKTT | | | | TKSFNRGEC | | |
| | | | PPVLDSDGSFFLYSKLTVD | | | | | | |
| | | | KSRWQQGNVFSCSVMHE | | | | | | |
| | | | ALHNHYTQKSLSLSPGK | | | | | | |
| EPI845 | EpCAM | 138 | QVQLVQSGPEVKKPGASV | 139 | QVQLVQSGPE | 140 | DIVMTQSPL | 141 | DIVMTQSPL |
| | | | KVSCKASGYTFTNYGMN | | VKKPGASVK | | SLPVTPGEPA | | SLPVTPGEP |
| | | | WVRQAPGQGLEWMGWIN | | VSCKASGYTF | | SISCRSSKNLL | | ASISCRSSK |
| | | | TYTGEPTYGEDFKGRFAFS | | TNYGMNWVR | | HSNGITYLYW | | NLLHSNGIT |
| | | | LDTSASTAYMELSSLRSED | | QAPGQGLEW | | YLQKPGQSPQ | | YLYWYLQK |
| | | | TAVYFCARFGNYVDYWG | | MGWINTYTG | | LLIYQMSNLA | | PGQSPQLLI |
| | | | QGSLVTVSSASTKGPSVFP | | EPTYGEDFKG | | SGVPDRFSSS | | YQMSNLAS |
| | | | LAPSSKSTSGGTAALGCLV | | RFAFSLDTSA | | GSGTDFTLKIS | | GVPDRFSSS |
| | | | KDYFPEPVTVSWNSGALT | | STAYMELSSL | | RVEAEDVGV | | GSGTDFTLK |
| | | | SGVHTFPAVLQSSGLYSLS | | RSEDTAVYFC | | YYCAQNLEIP | | ISRVEAEDV |
| | | | SVVTVPSSSLGTQTYICNV | | ARFGNYVDY | | RTFGQGTKVE | | GVYYCAQN |
| | | | NHKPSNTKVDKKVEPKSC | | WGQGSLVTV | | IKRTVAAPSV | | LEIPRTFGQ |
| | | | DKTHTCPPCPAPELLGGPS | | SS | | FIFPPSDEQLK | | GTKVEIK |
| | | | VFLFPPKPKDTLMISRTPE | | | | SGTASVVCLL | | |
| | | | VTCVVVDVSHEDPEVKFN | | | | NNFYPREAKV | | |
| | | | WYVDGVEVHNAKTKPRE | | | | QWKVDNALQ | | |
| | | | EQYNSTYRVVSVLTVLHQ | | | | SGNSQESVTE | | |
| | | | DWLNGKEYKCKVSNKAL | | | | QDSKDSTYSL | | |
| | | | PAPIEKTISKAKGQPREPQ | | | | SSTLTLSKAD | | |
| | | | VYTLPPSRDELTKNQVSL | | | | YEKHKVYAC | | |
| | | | WCLVKGFYPSDIAVEWES | | | | EVTHQGLSSP | | |
| | | | NGQPENNYKTTPPVLDSD | | | | VTKSFNRGEC | | |
| | | | GSFFLYSKLTVDKSRWQQ | | | | | | |
| | | | GNVFSCSVMHEALHNHYT | | | | | | |
| | | | QKSLSLSPGK | | | | | | |
| EPI846 | EpCAM | 142 | QIQLVQSGPELKKPGETV | 143 | QIQLVQSGPE | 144 | DIVMTQAAF | 145 | DIVMTQAA |
| | | | KISCKASGYTFTKYGMNW | | LKKPGETVKI | | SNPVTLGTSG | | FSNPVTLGT |
| | | | VKQAPGKGLKWMGWINT | | SCKASGYTFT | | SISCRSSKSLL | | SGSISCRSSK |
| | | | YTEEPTYGDDFKGRFAFSL | | KYGMNWVK | | HSNGITYLYW | | SLLHSNGIT |
| | | | ETSASTANLQINNLKSEDT | | QAPGKGLKW | | YLQKPGQSPQ | | YLYWYLQK |
| | | | ATYFCARFGSAVDYWGQ | | MGWINTYTE | | LLIYQMSNLA | | PGQSPQLLI |
| | | | GTSVTVSSASTKGPSVFPL | | EPTYGDDFKG | | SGVPDRFSSS | | YQMSNLAS |
| | | | APSSKSTSGGTAALGCLV | | RFAFSLETSAS | | GSGTDFTLRIS | | GVPDRFSSS |
| | | | KDYFPEPVTVSWNSGALT | | TANLQINNLK | | RVEAEDVGV | | GSGTDFTLR |
| | | | SGVHTFPAVLQSSGLYSLS | | SEDTATYFCA | | YYCAQNLELP | | ISRVEAEDV |
| | | | SVVTVPSSSLGTQTYICNV | | RFGSAVDYW | | RTFGGGTKLE | | GVYYCAQN |
| | | | NHKPSNTKVDKKVEPKSC | | GQGTSVTVSS | | IKRTVAAPSV | | LELPRTFGG |
| | | | DKTHTCPPCPAPELLGGPS | | | | FIFPPSDEQLK | | GTKLEIK |
| | | | VFLFPPKPKDTLMISRTPE | | | | SGTASVVCLL | | |
| | | | VTCVVVDVSHEDPEVKFN | | | | NNFYPREAKV | | |
| | | | WYVDGVEVHNAKTKPRE | | | | QWKVDNALQ | | |
| | | | EQYNSTYRVVSVLTVLHQ | | | | SGNSQESVTE | | |
| | | | DWLNGKEYKCKVSNKAL | | | | QDSKDSTYSL | | |
| | | | PAPIEKTISKAKGQPREPQ | | | | SSTLTLSKAD | | |
| | | | VYTLPPSRDELTKNQVSL | | | | YEKHKVYAC | | |
| | | | WCLVKGFYPSDIAVEWES | | | | EVTHQGLSSP | | |
| | | | NGQPENNYKTTPPVLDSD | | | | VTKSFNRGEC | | |
| | | | GSFFLYSKLTVDKSRWQQ | | | | | | |
| | | | GNVFSCSVMHEALHNHYT | | | | | | |
| | | | QKSLSLSPGK | | | | | | |
| EPI847 | EpCAM | 146 | EVQLVQSGPGLVQPGGSV | 147 | EVQLVQSGPG | 148 | DIQMTQSPS | 149 | DIQMTQSPS |
| | | | RISCAASGYTFTNYGMNW | | LVQPGGSVRI | | SLSASVGDRV | | SLSASVGDR |
| | | | VKQAPGKGLEWMGWINT | | SCAASGYTFT | | TITCRSTKSLL | | VTITCRSTK |
| | | | YTGESTYADSFKGRFTFSL | | NYGMNWVK | | HSNGITYLYW | | SLLHSNGIT |
| | | | DTSASAAYLQINSLRAEDT | | QAPGKGLEW | | YQQKPGKAP | | YLYWYQQK |
| | | | AVYYCARFAIKGDYWGQ | | MGWINTYTG | | KLLIYQMSNL | | PGKAPKLLI |
| | | | GTLLTVSSASTKGPSVFPL | | ESTYADSFKG | | ASGVPSRFSSS | | YQMSNLAS |
| | | | APSSKSTSGGTAALGCLV | | RFTFSLDTSAS | | GSGTDFTLTIS | | GVPSRFSSS |
| | | | KDYFPEPVTVSWNSGALT | | AAYLQINSLR | | SLQPEDFATY | | GSGTDFTLT |
| | | | SGVHTFPAVLQSSGLYSLS | | AEDTAVYYC | | YCAQNLEIPR | | ISSLQPEDFA |
| | | | SVVTVPSSSLGTQTYICNV | | ARFAIKGDY | | TFGQGTKVEL | | TYYCAQNL |
| | | | NHKPSNTKVDKKVEPKSC | | WGQGTLLTV | | KRTVAAPSVF | | EIPRTFGQG |
| | | | DKTHTCPPCPAPELLGGPS | | SS | | IFPPSDEQLKS | | TKVELK |
| | | | VFLFPPKPKDTLMISRTPE | | | | GTASVVCLLN | | |
| | | | VTCVVVDVSHEDPEVKFN | | | | NFYPREAKVQ | | |
| | | | WYVDGVEVHNAKTKPRE | | | | WKVDNALQS | | |
| | | | EQYNSTYRVVSVLTVLHQ | | | | GNSQESVTEQ | | |
| | | | DWLNGKEYKCKVSNKAL | | | | DSKDSTYSLS | | |
| | | | PAPIEKTISKAKGQPREPQ | | | | STLTLSKADY | | |
| | | | VYTLPPSRDELTKNQVSL | | | | EKHKVYACE | | |
| | | | WCLVKGFYPSDIAVEWES | | | | VTHQGLSSPV | | |
| | | | NGQPENNYKTTPPVLDSD | | | | TKSFNRGEC | | |
| | | | GSFFLYSKLTVDKSRWQQ | | | | | | |
| | | | GNVFSCSVMHEALHNHYT | | | | | | |
| | | | QKSLSLSPGK | | | | | | |
| EPI849 | EpCAM | 154 | QVQLVQSGAEVKKPGSSV | 155 | QVQLVQSGA | 156 | EIVMTQSPA | 157 | EIVMTQSPA |
| | | | KVSCKASGGTFSSYAISW | | EVKKPGSSVK | | TLSVSPGERA | | TLSVSPGER |
| | | | VRQAPGQGLEWMGGIIPIF | | VSCKASGGTF | | TLSCRASQSV | | ATLSCRASQ |
| | | | GTANYAQKFQGRVTITAD | | SSYAISWVRQ | | SSNLAWYQQ | | SVSSNLAW |
| | | | ESTSTAYMELSSLRSEDTA | | APGQGLEWM | | KPGQAPRLIIY | | YQQKPGQA |
| | | | VYYCARGLLWNYWGQGT | | GGIIPIFGTAN | | GASTTASGIP | | PRLIIYGAST |
| | | | LVTVSSASTKGPSVFPLAP | | YAQKFQGRV | | ARFSASGSGT | | TASGIPARF |
| | | | SSKSTSGGTAALGCLVKD | | TITADESTSTA | | DFTLTISSLQS | | SASGSGTDF |
| | | | YFPEPVTVSWNSGALTSG | | YMELSSLRSE | | EDFAVYYCQ | | TLTISSLQSE |
| | | | VHTFPAVLQSSGLYSLSSV | | DTAVYYCAR | | QYNNWPPAY | | DFAVYYCQ |
| | | | VTVPSSSLGTQTYICNVNH | | GLLWNYWGQ | | TFGQGTKLEI | | QYNNWPPA |
| | | | KPSNTKVDKKVEPKSCDK | | GTLVTVSS | | KRTVAAPSVF | | YTFGQGTKL |
| | | | THTCPPCPAPELLGGPSVF | | | | IFPPSDEQLKS | | EIK |
| | | | LFPPKPKDTLMISRTPEVT | | | | GTASVVCLLN | | |
| | | | CVVVDVSHEDPEVKFNW | | | | NFYPREAKVQ | | |
| | | | YVDGVEVHNAKTKPREEQ | | | | WKVDNALQS | | |
| | | | YNSTYRVVSVLTVLHQD | | | | GNSQESVTEQ | | |
| | | | WLNGKEYKCKVSNKALP | | | | DSKDSTYSLS | | |
| | | | APIEKTISKAKGQPREPQV | | | | STLTLSKADY | | |
| | | | YTLPPSRDELTKNQVSLW | | | | EKHKVYACE | | |
| | | | CLVKGFYPSDIAVEWESN | | | | VTHQGLSSPV | | |
| | | | GQPENNYKTTPPVLDSDG | | | | TKSFNRGEC | | |
| | | | SFFLYSKLTVDKSRWQQG | | | | | | |
| | | | NVFSCSVMHEALHNHYTQ | | | | | | |
| | | | KSLSLSPGK | | | | | | |
| EPI850 | TNFRSF10B | 158 | EVQLVESGGGLVQPGGSL | 159 | EVQLVESGGG | 160 | DIQMTQSPS | 161 | DIQMTQSPS |
| | | | RLSCAASGFTFSSYVMSW | | LVQPGGSLRL | | SLSASVGDRV | | SLSASVGDR |
| | | | VRQAPGKGLEWVATISSG | | SCAASGFTFS | | TITCKASQDV | | VTITCKASQ |
| | | | GSYTYYPDSVKGRFTISRD | | SYVMSWVRQ | | GTAVAWYQQ | | DVGTAVAW |
| | | | NAKNTLYLQMNSLRAEDT | | APGKGLEWV | | KPGKAPKLLI | | YQQKPGKA |
| | | | AVYYCARRGDSMITTDY | | ATISSGGSYT | | YWASTRHTG | | PKLLIYWAS |
| | | | WGQGTLVTVSSASTKGPS | | YYPDSVKGRF | | VPSRFSGSGS | | TRHTGVPSR |
| | | | VFPLAPSSKSTSGGTAALG | | TISRDNAKNT | | GTDFTLTISSL | | FSGSGSGTD |
| | | | CLVKDYFPEPVTVSWNSG | | LYLQMNSLR | | QPEDFATYYC | | FTLTISSLQP |
| | | | ALTSGVHTFPAVLQSSGLY | | AEDTAVYYC | | QQYSSYRTFG | | EDFATYYC |
| | | | SLSSVVTVPSSSLGTQTYIC | | ARRGDSMITT | | QGTKVEIKRT | | QQYSSYRTF |
| | | | NVNHKPSNTKVDKKVEPK | | DYWGQGTLV | | VAAPSVFIFPP | | GQGTKVEIK |
| | | | SCDKTHTCPPCPAPELLGG | | TVSS | | SDEQLKSGTA | | |
| | | | PSVFLFPPKPKDTLMISRTP | | | | SVVCLLNNFY | | |
| | | | EVTCVVVDVSHEDPEVKF | | | | PREAKVQWK | | |
| | | | NWYVDGVEVHNAKTKPR | | | | VDNALQSGN | | |
| | | | EEQYNSTYRVVSVLTVLH | | | | SQESVTEQDS | | |
| | | | QDWLNGKEYKCKVSNKA | | | | KDSTYSLSST | | |
| | | | LPAPIEKTISKAKGQPREPQ | | | | LTLSKADYEK | | |
| | | | VYTLPPSRDELTKNQVSL | | | | HKVYACEVT | | |
| | | | WCLVKGFYPSDIAVEWES | | | | HQGLSSPVTK | | |
| | | | NGQPENNYKTTPPVLDSD | | | | SFNRGEC | | |
| | | | GSFFLYSKLTVDKSRWQQ | | | | | | |
| | | | GNVFSCSVMHEALHNHYT | | | | | | |
| | | | QKSLSLSPGK | | | | | | |
| EPI851 | TNFRSF10B | 162 | EVQLVQSGGGVERPGGSL | 163 | EVQLVQSGG | 164 | SELTQDPAV | 165 | SELTQDPAV |
| | | | RLSCAASGFTFDDYAMSW | | GVERPGGSLR | | SVALGQTVRI | | SVALGQTV |
| | | | VRQAPGKGLEWVSGINW | | LSCAASGFTF | | TCSGDSLRSY | | RITCSGDSL |
| | | | QGGSTGYADSVKGRVTIS | | DDYAMSWVR | | YASWYQQKP | | RSYYASWY |
| | | | RDNAKNSLYLQMNSLRAE | | QAPGKGLEW | | GQAPVLVIYG | | QQKPGQAP |
| | | | DTAVYYCAKILGAGRGW | | VSGINWQGGS | | ANNRPSGIPD | | VLVIYGANN |
| | | | YFDYWGKGTTVTVSSAST | | TGYADSVKG | | RFSGSSSGNT | | RPSGIPDRFS |
| | | | KGPSVFPLAPSSKSTSGGT | | RVTISRDNAK | | ASLTITGAQA | | GSSSGNTAS |
| | | | AALGCLVKDYFPEPVTVS | | NSLYLQMNSL | | EDEADYYCN | | LTITGAQAE |
| | | | WNSGALTSGVHTFPAVLQ | | RAEDTAVYY | | SADSSGNHVV | | DEADYYCN |
| | | | SSGLYSLSSVVTVPSSSLG | | CAKILGAGRG | | FGGGTKLTVL | | SADSSGNH |
| | | | TQTYICNVNHKPSNTKVD | | WYFDYWGK | | RTVAAPSVFIF | | VVFGGGTK |
| | | | KKVEPKSCDKTHTCPPCP | | GTTVTVSS | | PPSDEQLKSG | | LTVL |
| | | | APELLGGPSVFLFPPKPKD | | | | TASVVCLLNN | | |
| | | | TLMISRTPEVTCVVVDVSH | | | | FYPREAKVQ | | |
| | | | EDPEVKFNWYVDGVEVH | | | | WKVDNALQS | | |
| | | | NAKTKPREEQYNSTYRVV | | | | GNSQESVTEQ | | |
| | | | SVLTVLHQDWLNGKEYK | | | | DSKDSTYSLS | | |
| | | | CKVSNKALPAPIEKTISKA | | | | STLTLSKADY | | |
| | | | KGQPREPQVYTLPPSRDEL | | | | EKHKVYACE | | |
| | | | TKNQVSLWCLVKGFYPSD | | | | VTHQGLSSPV | | |
| | | | IAVEWESNGQPENNYKTT | | | | TKSFNRGEC | | |
| | | | PPVLDSDGSFFLYSKLTVD | | | | | | |
| | | | KSRWQQGNVFSCSVMHE | | | | | | |
| | | | ALHNHYTQKSLSLSPGK | | | | | | |
| EPI852 | TNFRSF10B | 166 | EVQLQQSGAEVVKPGASV | 167 | EVQLQQSGAE | 168 | EIVMTQSPA | 169 | EIVMTQSPA |
| | | | KLSCKASGFNIKDTFIHWV | | VVKPGASVK | | TLSVSPGERA | | TLSVSPGER |
| | | | KQAPGQGLEWIGRIDPAN | | LSCKASGFNI | | TLSCRASQSIS | | ATLSCRASQ |
| | | | TNTKYDPKFQGKATITTDT | | KDTFIHWVK | | NNLHWYQQK | | SISNNLHWY |
| | | | SSNTAYMELSSLRSEDTAV | | QAPGQGLEWI | | PGQAPRLLIK | | QQKPGQAP |
| | | | YYCVRGLYTYYFDYWGQ | | GRIDPANTNT | | FASQSITGIPA | | RLLIKFASQ |
| | | | GTLVTVSSASTKGPSVFPL | | KYDPKFQGK | | RFSGSGSGTE | | SITGIPARFS |
| | | | APSSKSTSGGTAALGCLV | | ATITTDTSSNT | | FTLTISSLQSE | | GSGSGTEFT |
| | | | KDYFPEPVTVSWNSGALT | | AYMELSSLRS | | DFAVYYCQQ | | LTISSLQSED |
| | | | SGVHTFPAVLQSSGLYSLS | | EDTAVYYCV | | GNSWPYTFG | | FAVYYCQQ |
| | | | SVVTVPSSSLGTQTYICNV | | RGLYTYYFD | | QGTKLEIKRT | | GNSWPYTF |
| | | | NHKPSNTKVDKKVEPKSC | | YWGQGTLVT | | VAAPSVFIFPP | | GQGTKLEIK |
| | | | DKTHTCPPCPAPELLGGPS | | VSS | | SDEQLKSGTA | | |
| | | | VFLFPPKPKDTLMISRTPE | | | | SVVCLLNNFY | | |
| | | | VTCVVVDVSHEDPEVKFN | | | | PREAKVQWK | | |
| | | | WYVDGVEVHNAKTKPRE | | | | VDNALQSGN | | |
| | | | EQYNSTYRVVSVLTVLHQ | | | | SQESVTEQDS | | |
| | | | DWLNGKEYKCKVSNKAL | | | | KDSTYSLSST | | |
| | | | PAPIEKTISKAKGQPREPQ | | | | LTLSKADYEK | | |
| | | | VYTLPPSRDELTKNQVSL | | | | HKVYACEVT | | |
| | | | WCLVKGFYPSDIAVEWES | | | | HQGLSSPVTK | | |
| | | | NGQPENNYKTTPPVLDSD | | | | SFNRGEC | | |
| | | | GSFFLYSKLTVDKSR WQQ | | | | | | |
| | | | GNVFSCSVMHEALHNHYT | | | | | | |
| | | | QKSLSLSPGK | | | | | | |
| EPI853 | TNFRSF10B | 170 | QVQLQESGPGLVKPSQTL | 171 | QVQLQESGPG | 172 | EIVLTQSPGT | 173 | EIVLTQSPG |
| | | | SLTCTVSGGSISSGDYFWS | | LVKPSQTLSL | | LSLSPGERAT | | TLSLSPGER |
| | | | WIRQLPGKGLEWIGHIHNS | | TCTVSGGSISS | | LSCRASQGIS | | ATLSCRASQ |
| | | | GTTYYNPSLKSRVTISVDT | | GDYFWS WIR | | RSYLAWYQQ | | GISRSYLAW |
| | | | SKKQFSLRLSSVTAADTA | | QLPGKGLEWI | | KPGQAPSLLI | | YQQKPGQA |
| | | | VYYCARDRGGDYYYGMD | | GHIHNSGTTY | | YGASSRATGI | | PSLLIYGASS |
| | | | VWGQGTTVTVSSASTKGP | | YNPSLKSRVT | | PDRFSGSGSG | | RATGIPDRF |
| | | | SVFPLAPSSKSTSGGTAAL | | ISVDTSKKQF | | TDFTLTISRLE | | SGSGSGTDF |
| | | | GCLVKDYFPEPVTVSWNS | | SLRLSSVTAA | | PEDFAVYYCQ | | TLTISRLEPE |
| | | | GALTSGVHTFPAVLQSSGL | | DTAVYYCAR | | QFGSSPWTFG | | DFAVYYCQ |
| | | | YSLSSVVTVPSSSLGTQTYI | | DRGGDYYYG | | QGTKVEIKRT | | QFGSSPWTF |
| | | | CNVNHKPSNTKVDKKVEP | | MDVWGQGTT | | VAAPSVFIFPP | | GQGTKVEIK |
| | | | KSCDKTHTCPPCPAPELLG | | VTVSS | | SDEQLKSGTA | | |
| | | | GPSVFLFPPKPKDTLMISR | | | | SVVCLLNNFY | | |
| | | | TPEVTCVVVDVSHEDPEV | | | | PREAKVQWK | | |
| | | | KFNWYVDGVEVHNAKTK | | | | VDNALQSGN | | |
| | | | PREEQYNSTYRVVSVLTV | | | | SQESVTEQDS | | |
| | | | LHQDWLNGKEYKCKVSN | | | | KDSTYSLSST | | |
| | | | KALPAPIEKTISKAKGQPR | | | | LTLSKADYEK | | |
| | | | EPQVYTLPPSRDELTKNQV | | | | HKVYACEVT | | |
| | | | SLWCLVKGFYPSDIAVEW | | | | HQGLSSPVTK | | |
| | | | ESNGQPENNYKTTPPVLDS | | | | SFNRGEC | | |
| | | | DGSFFLYSKLTVDKSRWQ | | | | | | |
| | | | QGNVFSCSVMHEALHNH | | | | | | |
| | | | YTQKSLSLSPGK | | | | | | |
| EPI854 | TNFRSF10B | 174 | EVQLVQSGGGVERPGGSL | 175 | EVQLVQSGG | 176 | SSELTQDPA | 177 | SSELTQDPA |
| | | | RLSCAASGFTFDDYGMSW | | GVERPGGSLR | | VSVALGQTV | | VSVALGQT |
| | | | VRQAPGKGLEWVSGINW | | LSCAASGFTF | | RITCQGDSLR | | VRITCQGDS |
| | | | NGGSTGYADSVKGRVTIS | | DDYGMSWVR | | SYYASWYQQ | | LRSYYASW |
| | | | RDNAKNSLYLQMNSLRAE | | QAPGKGLEW | | KPGQAPVLVI | | YQQKPGQA |
| | | | DTAVYYCAKILGAGRGW | | VSGINWNGGS | | YGKNNRPSGI | | PVLVIYGKN |
| | | | YFDLWGKGTTVTVSSAST | | TGYADSVKG | | PDRFSGSSSG | | NRPSGIPDR |
| | | | KGPSVFPLAPSSKSTSGGT | | RVTISRDNAK | | NTASLTITGA | | FSGSSSGNT |
| | | | AALGCLVKDYFPEPVTVS | | NSLYLQMNSL | | QAEDEADYY | | ASLTITGAQ |
| | | | WNSGALTSGVHTFPAVLQ | | RAEDTAVYY | | CNSRDSSGNH | | AEDEADYY |
| | | | SSGLYSLSSVVTVPSSSLG | | CAKILGAGRG | | VVFGGGTKLT | | CNSRDSSGN |
| | | | TQTYICNVNHKPSNTKVD | | WYFDLWGKG | | VLRTVAAPSV | | HVVFGGGT |
| | | | KKVEPKSCDKTHTCPPCP | | TTVTVSS | | FIFPPSDEQLK | | KLTVL |
| | | | APELLGGPSVFLFPPKPKD | | | | SGTASVVCLL | | |
| | | | TLMISRTPEVTCVVVDVSH | | | | NNFYPREAKV | | |
| | | | EDPEVKFNWYVDGVEVH | | | | QWKVDNALQ | | |
| | | | NAKTKPREEQYNSTYRVV | | | | SGNSQESVTE | | |
| | | | SVLTVLHQDWLNGKEYK | | | | QDSKDSTYSL | | |
| | | | CKVSNKALPAPIEKTISKA | | | | SSTLTLSKAD | | |
| | | | KGQPREPQVYTLPPSRDEL | | | | YEKHKVYAC | | |
| | | | TKNQVSLWCLVKGFYPSD | | | | EVTHQGLSSP | | |
| | | | LAVEWESNGQPENNYKTT | | | | VTKSFNRGEC | | |
| | | | PPVLDSDGSFFLYSKLTVD | | | | | | |
| | | | KSRWQQGNVFSCSVMHE | | | | | | |
| | | | ALHNHYTQKSLSLSPGK | | | | | | |
| EPI855 | STEAP1 | 178 | EVQLVESGGGLVQPGGSL | 179 | EVQLVESGGG | 180 | DIQMTQSPS | 181 | DIQMTQSPS |
| | | | RLSCAVSGYSITSDYAWN | | LVQPGGSLRL | | SLSASVGDRV | | SLSASVGDR |
| | | | WVRQAPGKGLEWVGYIS | | SCAVSGYSITS | | TITCKSSQSLL | | VTITCKSSQ |
| | | | NSGSTSYNPSLKSRFTISRD | | DYAWNWVR | | YRSNQKNYL | | SLLYRSNQK |
| | | | TSKNTLYLQMNSLRAEDT | | QAPGKGLEW | | AWYQQKPGK | | NYLAWYQQ |
| | | | AVYYCARERNYDYDDYY | | VGYISNSGST | | APKLLIYWAS | | KPGKAPKLL |
| | | | YAMDYWGQGTLVTVSSA | | SYNPSLKSRF | | TRESGVPSRF | | IYWASTRES |
| | | | STKGPSVFPLAPSSKSTSG | | TISRDTSKNTL | | SGSGSGTDFT | | GVPSRFSGS |
| | | | GTAALGCLVKDYFPEPVT | | YLQMNSLRA | | LTISSLQPEDF | | GSGTDFTLT |
| | | | VSWNSGALTSGVHTFPAV | | EDTAVYYCA | | ATYYCQQYY | | ISSLQPEDFA |
| | | | LQSSGLYSLSSVVTVPSSS | | RERNYDYDD | | NYPRTFGQGT | | TYYCQQYY |
| | | | LGTQTYICNVNHKPSNTK | | YYYAMDYW | | KVEIKRTVAA | | NYPRTFGQ |
| | | | VDKKVEPKSCDKTHTCPP | | GQGTLVTVSS | | PSVFIFPPSDE | | GTKVEIK |
| | | | CPAPELLGGPSVFLFPPKP | | | | QLKSGTASVV | | |
| | | | KDTLMISRTPEVTCVVVD | | | | CLLNNFYPRE | | |
| | | | VSHEDPEVKFNWYVDGV | | | | AKVQWKVDN | | |
| | | | EVHNAKTKPREEQYNSTY | | | | ALQSGNSQES | | |
| | | | RVVSVLTVLHQDWLNGK | | | | VTEQDSKDST | | |
| | | | EYKCKVSNKALPAPIEKTI | | | | YSLSSTLTLSK | | |
| | | | SKAKGQPREPQVYTLPPSR | | | | ADYEKHKVY | | |
| | | | DELTKNQVSLWCLVKGFY | | | | ACEVTHQGLS | | |
| | | | PSDIAVEWESNGQPENNY | | | | SPVTKSFNRG | | |
| | | | KTTPPVLDSDGSFFLYSKL | | | | EC | | |
| | | | TVDKSRWQQGNVFSCSV | | | | | | |
| | | | MHEALHNHYTQKSLSLSP | | | | | | |
| | | | GK | | | | | | |
| EPI856 | ITGB6 | 182 | EVQLVESGGGLVQPGGSL | 183 | EVQLVESGGG | 184 | EIVLTQSPAT | 185 | EIVLTQSPA |
| | | | RLSCAASGFTFSRYVMSW | | LVQPGGSLRL | | LSLSPGERAT | | TLSLSPGER |
| | | | VRQAPGKGLEWVASISSG | | SCAASGFTFS | | LSCSASSSVSS | | ATLSCSASS |
| | | | GRMYYPDTVKGRFTISRD | | RYVMSWVRQ | | SYLYWYQQK | | SVSSSYLYW |
| | | | NAKNSLYLQMNSLRAEDT | | APGKGLEWV | | PGQAPRLLIY | | YQQKPGQA |
| | | | AVYYCARGSIYDGYYVFP | | ASISSGGRMY | | STSNLASGIPA | | PRLLIYSTSN |
| | | | YWGQGTLVTVSSASTKGP | | YPDTVKGRFT | | RFSGSGSGTD | | LASGIPARF |
| | | | SVFPLAPSSKSTSGGTAAL | | ISRDNAKNSL | | FTLTISSLEPE | | SGSGSGTDF |
| | | | GCLVKDYFPEPVTVSWNS | | YLQMNSLRA | | DFAVYYCHQ | | TLTISSLEPE |
| | | | GALTSGVHTFPAVLQSSGL | | EDTAVYYCA | | WSTYPPTFGG | | DFAVYYCH |
| | | | YSLSSVVTVPSSSLGTQTYI | | RGSIYDGYYV | | GTKVEIKRTV | | QWSTYPPTF |
| | | | CNVNHKPSNTKVDKKVEP | | FPYWGQGTL | | AAPSVFIFPPS | | GGGTKVEIK |
| | | | KSCDKTHTCPPCPAPELLG | | VTVSS | | DEQLKSGTAS | | |
| | | | GPSVFLFPPKPKDTLMISR | | | | VVCLLNNFYP | | |
| | | | TPEVTCVVVDVSHEDPEV | | | | REAKVQWKV | | |
| | | | KFNWYVDGVEVHNAKTK | | | | DNALQSGNS | | |
| | | | PREEQYNSTYRVVSVLTV | | | | QESVTEQDSK | | |
| | | | LHQDWLNGKEYKCKVSN | | | | DSTYSLSSTLT | | |
| | | | KALPAPIEKTISKAKGQPR | | | | LSKADYEKH | | |
| | | | EPQVYTLPPSRDELTKNQV | | | | KVYACEVTH | | |
| | | | SLWCLVKGFYPSDIAVEW | | | | QGLSSPVTKS | | |
| | | | ESNGQPENNYKTTPPVLDS | | | | FNRGEC | | |
| | | | DGSFFLYSKLTVDKSRWQ | | | | | | |
| | | | QGNVFSCSVMHEALHNH | | | | | | |
| | | | YTQKSLSLSPGK | | | | | | |
| EPI1010 | ITGB6 | 286 | QVQLVQSGAEVKKPGAS | 287 | QVQLVQSGA | 288 | DVVMTQSP | 289 | DVVMTQSP |
| | | | VKVSCKASGYSFSGYFMN | | EVKKPGASV | | LSLPVTLGQP | | LSLPVTLGQ |
| | | | WVRQAPGQGLEWMGLIN | | KVSCKASGYS | | ASISCKSSQSL | | PASISCKSSQ |
| | | | PYNGDSFYNQKFKGRVTM | | FSGYFMNWV | | LDSDGKTYLN | | SLLDSDGKT |
| | | | TRQTSTSTVYMELSSLRSE | | RQAPGQGLE | | WLFQRPGQSP | | YLNWLFQR |
| | | | DTAVYYCVRGLRRDFDY | | WMGLINPYN | | RRLIYLVSEL | | PGQSPRRLI |
| | | | WGQGTLVTVSSASTKGPS | | GDSFYNQKFK | | DSGVPDRFSG | | YLVSELDSG |
| | | | VFPLAPSSKSTSGGTAALG | | GRVTMTRQT | | SGSGTDFTLKI | | VPDRFSGSG |
| | | | CLVKDYFPEPVTVSWNSG | | STSTVYMELS | | SRVEAEDVG | | SGTDFTLKI |
| | | | ALTSGVHTFPAVLQSSGLY | | SLRSEDTAVY | | VYYCWQGTH | | SRVEAEDV |
| | | | SLSSVVTVPSSSLGTQTYIC | | YCVRGLRRDF | | FPRTFGGGTK | | GVYYCWQG |
| | | | NVNHKPSNTKVDKKVEPK | | DYWGQGTLV | | LEIKRTVAAP | | THFPRTFGG |
| | | | SCDKTHTCPPCPAPELLGG | | TVSS | | SVFIFPPSDEQ | | GTKLEIK |
| | | | PSVFLFPPKPKDTLMISRTP | | | | LKSGTASVVC | | |
| | | | EVTCVVVDVSHEDPEVKF | | | | LLNNFYPREA | | |
| | | | NWYVDGVEVHNAKTKPR | | | | KVQWKVDNA | | |
| | | | EEQYNSTYRVVSVLTVLH | | | | LQSGNSQESV | | |
| | | | QDWLNGKEYKCKVSNKA | | | | TEQDSKDSTY | | |
| | | | LPAPIEKTISKAKGQPREPQ | | | | SLSSTLTLSKA | | |
| | | | VYTLPPSRDELTKNQVSL | | | | DYEKHKVYA | | |
| | | | WCLVKGFYPSDIAVEWES | | | | CEVTHQGLSS | | |
| | | | NGQPENNYKTTPPVLDSD | | | | PVTKSFNRGE | | |
| | | | GSFFLYSKLTVDKSRWQQ | | | | C | | |
| | | | GNVFSCSVMHEALHNHYT | | | | | | |
| | | | QKSLSLSPGKGGSHHHHHH | | | | | | |
| EPI858 | MELTF | 190 | QVQLVQSGAEVKKPGAS | 191 | QVQLVQSGA | 192 | DIQMTQSPS | 193 | DIQMTQSPS |
| | | | VKVSCKASGYTFTNYRIE | | EVKKPGASV | | SLSASVGDRV | | SLSASVGDR |
| | | | WVRQAPGQGLEWMGEIL | | KVSCKASGYT | | TITCRASQDIS | | VTITCRASQ |
| | | | PRGGNTNYNEKFKGRVTF | | FTNYRIEWVR | | NYLNWYQQK | | DISNYLNW |
| | | | TADTSTSTAYMELRSLRSD | | QAPGQGLEW | | PGKAPKLLIY | | YQQKPGKA |
| | | | DTAVYYCARDDGYYGRF | | MGEILPRGGN | | YTSRLHSGVP | | PKLLIYYTS |
| | | | AYWGQGTLVTVSSASTKG | | TNYNEKFKG | | SRFSGSGSGT | | RLHSGVPSR |
| | | | PSVFPLAPSSKSTSGGTAA | | RVTFTADTST | | DYTLTISSLQP | | FSGSGSGTD |
| | | | LGCLVKDYFPEPVTVSWN | | STAYMELRSL | | EDFATYYCQ | | YTLTISSLQP |
| | | | SGALTSGVHTFPAVLQSSG | | RSDDTAVYY | | QGNTLPPTFG | | EDFATYYC |
| | | | LYSLSSVVTVPSSSLGTQT | | CARDDGYYG | | GGTKVEIKRT | | QQGNTLPPT |
| | | | YICNVNHKPSNTKVDKKV | | RFAYWGQGT | | VAAPSVFIFPP | | FGGGTKVEI |
| | | | EPKSCDKTHTCPPCPAPEL | | LVTVSS | | SDEQLKSGTA | | K |
| | | | LGGPSVFLFPPKPKDTLMI | | | | SVVCLLNNFY | | |
| | | | SRTPEVTCVVVDVSHEDP | | | | PREAKVQWK | | |
| | | | EVKFNWYVDGVEVHNAK | | | | VDNALQSGN | | |
| | | | TKPREEQYNSTYRVVSVL | | | | SQESVTEQDS | | |
| | | | TVLHQDWLNGKEYKCKV | | | | KDSTYSLSST | | |
| | | | SNKALPAPIEKTISKAKGQ | | | | LTLSKADYEK | | |
| | | | PREPQVYTLPPSRDELTKN | | | | HKVYACEVT | | |
| | | | QVSLWCLVKGFYPSDIAV | | | | HQGLSSPVTK | | |
| | | | EWESNGQPENNYKTTPPV | | | | SFNRGEC | | |
| | | | LDSDGSFFLYSKLTVDKSR | | | | | | |
| | | | WQQGNVFSCSVMHEALH | | | | | | |
| | | | NHYTQKSLSLSPGK | | | | | | |
| EPI859 | MELTF | 194 | QVQLQESGPGLVKPSETLS | 195 | QVQLQESGPG | 196 | DFVMTQSPL | 197 | DFVMTQSPL |
| | | | LTCTVSGDSITSGYWNWIR | | LVKPSETLSL | | SLPVTLGQPA | | SLPVTLGQP |
| | | | QPPGKGLEYIGYISDSGITY | | TCTVSGDSITS | | SISCRASQSLV | | ASISCRASQ |
| | | | YNPSLKSRVTISRDTSKNQ | | GYWNWIRQP | | HSDGNTYLH | | SLVHSDGNT |
| | | | YSLKLSSVTAADTAVYYC | | PGKGLEYIGY | | WYQQRPGQS | | YLHWYQQR |
| | | | ARRTLATYYAMDYWGQG | | ISDSGITYYNP | | PRLLIYRVSN | | PGQSPRLLI |
| | | | TLVTVSSASTKGPSVFPLA | | SLKSRVTISRD | | RFSGVPDRFS | | YRVSNRFSG |
| | | | PSSKSTSGGTAALGCLVK | | TSKNQYSLKL | | GSGSGTDFTL | | VPDRFSGSG |
| | | | DYFPEPVTVSWNSGALTS | | SSVTAADTAV | | KISRVEAEDV | | SGTDFTLKI |
| | | | GVHTFPAVLQSSGLYSLSS | | YYCARRTLAT | | GVYYCSQSTH | | SRVEAEDV |
| | | | VVTVPSSSLGTQTYICNVN | | YYAMDYWG | | VPPTFGQGTK | | GVYYCSQS |
| | | | HKPSNTKVDKKVEPKSCD | | QGTLVTVSS | | LEIKRTVAAP | | THVPPTFGQ |
| | | | KTHTCPPCPAPELLGGPSV | | | | SVFIFPPSDEQ | | GTKLEIK |
| | | | FLFPPKPKDTLMISRTPEVT | | | | LKSGTASVVC | | |
| | | | CVVVDVSHEDPEVKFNW | | | | LLNNFYPREA | | |
| | | | YVDGVEVHNAKTKPREEQ | | | | KVQWKVDNA | | |
| | | | YNSTYRVVSVLTVLHQD | | | | C | | |
| | | | WLNGKEYKCKVSNKALP | | | | LQSGNSQESV | | |
| | | | APIEKTISKAKGQPREPQV | | | | TEQDSKDSTY | | |
| | | | YTLPPSRDELTKNQVSLW | | | | SLSSTLTLSKA | | |
| | | | CLVKGFYPSDIAVEWESN | | | | DYEKHKVYA | | |
| | | | GQPENNYKTTPPVLDSDG | | | | CEVTHQGLSS | | |
| | | | SFFLYSKLTVDKSRWQQG | | | | PVTKSFNRGE | | |
| | | | NVFSCSVMHEALHNHYTQ | | | | | | |
| | | | KSLSLSPGK | | | | | | |
| EPI808 | TRQP2 | 14 | QVQLQQSGSELKKPGASV | 15 | QVQLQQSGSE | 16 | DIQLTQSPSS | 17 | DIQLTQSPSS |
| | | | KVSCKASGYTFTNYGMN | | LKKPGASVK | | LSASVGDRVS | | LSASVGDR |
| | | | WVKQAPGQGLKWMGWI | | VSCKASGYTF | | ITCKASQDVSI | | VSITCKASQ |
| | | | NTYTGEPTYTDDFKGRFA | | TNYGMNWV | | AVAWYQQKP | | DVSIAVAW |
| | | | FSLDTSVSTAYLQISSLKA | | KQAPGQGLK | | GKAPKLLIYS | | YQQKPGKA |
| | | | DDTAVYFCARGGFGSSYW | | WMGWINTYT | | ASYRYTGVPD | | PKLLIYSAS |
| | | | YFDVWGQGSLVTVSSAST | | GEPTYTDDFK | | RFSGSGSGTD | | YRYTGVPD |
| | | | KGPSVFPLAPSSKSTSGGT | | GRFAFSLDTS | | FTLTISSLQPE | | RFSGSGSGT |
| | | | AALGCLVKDYFPEPVTVS | | VSTAYLQISSL | | DFAVYYCQQ | | DFTLTISSLQ |
| | | | WNSGALTSGVHTFPAVLQ | | KADDTAVYF | | HYITPLTFGA | | PEDFAVYY |
| | | | SSGLYSLSSVVTVPSSSLG | | CARGGFGSSY | | GTKVEIKRTV | | CQQHYITPL |
| | | | TQTYICNVNHKPSNTKVD | | WYFDVWGQ | | AAPSVFIFPPS | | TFGAGTKVE |
| | | | KKVEPKSCDKTHTCPPCP | | GSLVTVSS | | DEQLKSGTAS | | K |
| | | | APELLGGPSVFLFPPKPKD | | | | VVCLLNNFYP | | |
| | | | TLMISRTPEVTCVVVDVSH | | | | REAKVQWKV | | |
| | | | EDPEVKFNWYVDGVEVH | | | | DNALQSGNS | | |
| | | | NAKTKPREEQYNSTYRVV | | | | QESVTEQDSK | | |
| | | | SVLTVLHQDWLNGKEYK | | | | DSTYSLSSTLT | | |
| | | | CKVSNKALPAPIEKTISKA | | | | LSKADYEKH | | |
| | | | KGQPREPQVYTLPPSRDEL | | | | KVYACEVTH | | |
| | | | TKNQVSLWCLVKGFYPSD | | | | QGLSSPVTKS | | |
| | | | IAVEWESNGQPENNYKTT | | | | FNRGEC | | |
| | | | PPVLDSDGSFFLYSKLTVD | | | | | | |
| | | | KSRWQQGNVFSCSVMHE | | | | | | |
| | | | ALHNHYTQKSLSLSPGK | | | | | | |
| EPI832 | CEACAM5 | 86 | QVQLVQSGAEVKKPGAS | 87 | QVQLVQSGA | 88 | DIQMTQSPS | 89 | DIQMTQSPS |
| | | | VKVSCKASGYTFTEFGMN | | EVKKPGASV | | SLSASVGDRV | | SLSASVGDR |
| | | | WVRQAPGQGLEWMGWIN | | KVSCKASGYT | | TITCKASAAV | | VTITCKASA |
| | | | TKTGEATYVEEFKGRVTF | | FTEFGMNWV | | GTYVAWYQQ | | AVGTYVAW |
| | | | TTDTSTSTAYMELRSLRSD | | RQAPGQGLE | | KPGKAPKLLI | | YQQKPGKA |
| | | | DTAVYYCARWDFAYYVE | | WMGWINTKT | | YSASYRKRG | | PKLLIYSAS |
| | | | AMDYWGQGTTVTVSSAS | | GEATYVEEFK | | VPSRFSGSGS | | YRKRGVPS |
| | | | TKGPSVFPLAPSSKSTSGG | | GRVTFTTDTS | | GTDFTLTISSL | | RFSGSGSGT |
| | | | TAALGCLVKDYFPEPVTV | | TSTAYMELRS | | QPEDFATYYC | | DFTLTISSLQ |
| | | | SWNSGALTSGVHTFPAVL | | LRSDDTAVY | | HQYYTYPLFT | | PEDFATYYC |
| | | | QSSGLYSLSSVVTVPSSSL | | YCARWDFAY | | FGQGTKLEIK | | HQYYTYPLF |
| | | | GTQTYICNVNHKPSNTKV | | YVEAMDYW | | RTVAAPSVFIF | | TFGQGTKLE |
| | | | DKKVEPKSCDKTHTCPPC | | GQGTTVTVSS | | PPSDEQLKSG | | IK |
| | | | PAPELLGGPSVFLFPPKPK | | | | TASVVCLLNN | | |
| | | | DTLMISRTPEVTCVVVDVS | | | | FYPREAKVQ | | |
| | | | HEDPEVKFNWYVDGVEV | | | | WKVDNALQS | | |
| | | | HNAKTKPREEQYNSTYRV | | | | GNSQESVTEQ | | |
| | | | VSVLTVLHQDWLNGKEY | | | | DSKDSTYSLS | | |
| | | | KCKVSNKALPAPIEKTISK | | | | STLTLSKADY | | |
| | | | AKGQPREPQVYTLPPSRDE | | | | EKHKVYACE | | |
| | | | LTKNQVSLWCLVKGFYPS | | | | VTHQGLSSPV | | |
| | | | DIAVEWESNGQPENNYKT | | | | TKSFNRGEC | | |
| | | | TPPVLDSDGSFFLYSKLTV | | | | | | |
| | | | DKSRWQQGNVFSCSVMH | | | | | | |
| | | | EALHNHYTQKSLSLSPGK | | | | | | |
| EPI833 | CEACAM5 | 90 | EVKLVESGGGLVQPGGSL | 91 | EVKLVESGGG | 92 | QTVLSQSPA | 93 | QTVLSQSPA |
| | | | RLSCATSGFTFTDYYMNW | | LVQPGGSLRL | | ILSASPGEKVT | | ILSASPGEK |
| | | | VRQPPGKALEWLGFIGNK | | SCATSGFTFT | | MTCRASSSVT | | VTMTCRAS |
| | | | ANGYTTEYSASVKGRFTIS | | DYYMNWVR | | YIHWYQQKP | | SSVTYIHWY |
| | | | RDKSQSILYLQMNTLRAE | | QPPGKALEW | | GSSPKSWIYA | | QQKPGSSPK |
| | | | DSATYYCTRDRGLRFYFD | | LGFIGNKANG | | TSNLASGVPA | | SWIYATSNL |
| | | | YWGQGTTLTVSSASTKGP | | YTTEYSASVK | | RFSGSGSGTS | | ASGVPARFS |
| | | | SVFPLAPSSKSTSGGTAAL | | GRFTISRDKS | | YSLTISRVEAE | | GSGSGTSYS |
| | | | GCLVKDYFPEPVTVSWNS | | QSILYLQMNT | | DAATYYCQH | | LTISRVEAE |
| | | | GALTSGVHTFPAVLQSSGL | | LRAEDSATYY | | WSSKPPTFGG | | DAATYYCQ |
| | | | YSLSSVVTVPSSSLGTQTYI | | CTRDRGLRFY | | GTKLEIKRTV | | HWSSKPPTF |
| | | | CNVNHKPSNTKVDKKVEP | | FDYWGQGTT | | AAPSVFIFPPS | | GGGTKLEIK |
| | | | KSCDKTHTCPPCPAPELLG | | LTVSS | | DEQLKSGTAS | | |
| | | | GPSVFLFPPKPKDTLMISR | | | | VVCLLNNFYP | | |
| | | | TPEVTCVVVDVSHEDPEV | | | | REAKVQWKV | | |
| | | | KFNWYVDGVEVHNAKTK | | | | DNALQSGNS | | |
| | | | PREEQYNSTYRVVSVLTV | | | | QESVTEQDSK | | |
| | | | LHQDWLNGKEYKCKVSN | | | | DSTYSLSSTLT | | |
| | | | KALPAPIEKTISKAKGQPR | | | | LSKADYEKH | | |
| | | | EPQVYTLPPSRDELTKNQV | | | | KVYACEVTH | | |
| | | | SLWCLVKGFYPSDIAVEW | | | | QGLSSPVTKS | | |
| | | | ESNGQPENNYKTTPPVLDS | | | | FNRGEC | | |
| | | | DGSFFLYSKLTVDKSRWQ | | | | | | |
| | | | QGNVFSCSVMHEALHNH | | | | | | |
| | | | YTQKSLSLSPGK | | | | | | |
| EPI869 | CEACAM5 | 226 | QVQLQESGPELKKPGETV | 227 | QVQLQESGPE | 228 | SIVMTQTPL | 229 | SIVMTQTPL |
| | | | KISCKASGYTFRNYGMNW | | LKKPGETVKI | | SLPVSLGDQA | | SLPVSLGDQ |
| | | | VKQAPGKGLKWMGWINT | | SCKASGYTFR | | SISCQSSQSIV | | ASISCQSSQS |
| | | | YTGEPTYADDFKGRFAFS | | NYGMNWVK | | HSNGNTYLE | | IVHSNGNTY |
| | | | LETSASTAYLQINNVKNED | | QAPGKGLKW | | WYLQKPGQS | | LEWYLQKP |
| | | | TATYFCARKGWMDFNGS | | MGWINTYTG | | PNLLIYKVSN | | GQSPNLLIY |
| | | | SLDYWGQGTTVTVSSAST | | EPTYADDFKG | | RFSGVPDRFS | | KVSNRFSGV |
| | | | KGPSVFPLAPSSKSTSGGT | | RFAFSLETSAS | | GSGSGTDFTL | | PDRFSGSGS |
| | | | AALGCLVKDYFPEPVTVS | | TAYLQINNVK | | KISRVEAEDIG | | GTDFTLKIS |
| | | | WNSGALTSGVHTFPAVLQ | | NEDTATYFCA | | VYYCFQGSH | | RVEAEDIGV |
| | | | SSGLYSLSSVVTVPSSSLG | | RKGWMDFNG | | VPPTFGGGTK | | YYCFQGSH |
| | | | TQTYICNVNHKPSNTKVD | | SSLDYWGQG | | LEIKRTVAAP | | VPPTFGGGT |
| | | | KKVEPKSCDKTHTCPPCP | | TTVTVSS | | SVFIFPPSDEQ | | KLEIK |
| | | | APELLGGPSVFLFPPKPKD | | | | LKSGTASVVC | | |
| | | | TLMISRTPEVTCVVVDVSH | | | | LLNNFYPREA | | |
| | | | EDPEVKFNWYVDGVEVH | | | | KVQWKVDNA | | |
| | | | NAKTKPREEQYNSTYRVV | | | | LQSGNSQESV | | |
| | | | SVLTVLHQDWLNGKEYK | | | | TEQDSKDSTY | | |
| | | | CKVSNKALPAPIEKTISKA | | | | SLSSTLTLSKA | | |
| | | | KGQPREPQVYTLPPSRDEL | | | | DYEKHKVYA | | |
| | | | TKNQVSLWCLVKGFYPSD | | | | CEVTHQGLSS | | |
| | | | IAVEWESNGQPENNYKTT | | | | PVTKSFNRGE | | |
| | | | PPVLDSDGSFFLYSKLTVD | | | | C | | |
| | | | KSRWQQGNVFSCSVMHE | | | | | | |
| | | | ALHNHYTQKSLSLSPGK | | | | | | |
| EPI870 | CEACAM5 | 230 | EVRLVESGGGLVQGPGSL | 231 | EVRLVESGGG | 232 | DIQLTQSPAI | 233 | DIQLTQSPAI |
| | | | RLSCAASGFALTDYYMSW | | LVQGPGSLRL | | MSASPGEKVT | | MSASPGEK |
| | | | VRQSPGKTLEWLGFIANK | | SCAASGFALT | | MTCSASSRVS | | VTMTCSASS |
| | | | ANGHTTDYSPSVKGRFTIS | | DYYMSWVRQ | | YIHWYQQKS | | RVSYIHWY |
| | | | RDNSQTILYLQMNTLRTE | | SPGKTLEWLG | | GTSPKRWIYG | | QQKSGTSPK |
| | | | DSATYYCARDMGIRWNF | | FIANKANGHT | | TSTLASGVPA | | RWIYGTSTL |
| | | | DVWGQGTTVTVSSASTKG | | TDYSPSVKGR | | RFSGSGSGTS | | ASGVPARFS |
| | | | PSVFPLAPSSKSTSGGTAA | | FTISRDNSQTI | | YSLTISSMEA | | GSGSGTSYS |
| | | | LGCLVKDYFPEPVTVSWN | | LYLQMNTLR | | EDAATYYCQ | | LTISSMEAE |
| | | | SGALTSGVHTFPAVLQSSG | | TEDSATYYCA | | QWSYNPPTFG | | DAATYYCQ |
| | | | LYSLSSVVTVPSSSLGTQT | | RDMGIRWNF | | AGTKLELKRT | | QWSYNPPTF |
| | | | YICNVNHKPSNTKVDKKV | | DVWGQGTTV | | VAAPSVFIFPP | | GAGTKLEL |
| | | | EPKSCDKTHTCPPCPAPEL | | TVSS | | SDEQLKSGTA | | K |
| | | | LGGPSVFLFPPKPKDTLMI | | | | SVVCLLNNFY | | |
| | | | SRTPEVTCVVVDVSHEDP | | | | PREAKVQWK | | |
| | | | EVKFNWYVDGVEVHNAK | | | | VDNALQSGN | | |
| | | | TKPREEQYNSTYRVVSVL | | | | SQESVTEQDS | | |
| | | | TVLHQDWLNGKEYKCKV | | | | KDSTYSLSST | | |
| | | | SNKALPAPIEKTISKAKGQ | | | | LTLSKADYEK | | |
| | | | PREPQVYTLPPSRDELTKN | | | | HKVYACEVT | | |
| | | | QVSLWCLVKGFYPSDIAV | | | | HQGLSSPVTK | | |
| | | | EWESNGQPENNYKTTPPV | | | | SFNRGEC | | |
| | | | LDSDGSFFLYSKLTVDKSR | | | | | | |
| | | | WQQGNVFSCSVMHEALH | | | | | | |
| | | | NHYTQKSLSLSPGK | | | | | | |
| EPI871 | CEACAM5 | 234 | EVQLQESGPGLVKPSQTLS | 235 | EVQLQESGPG | 236 | EIVLTQSPAT | 237 | EIVLTQSPA |
| | | | LTCTVSDGSVSRGGYYLT | | LVKPSQTLSL | | LSVSPGERAT | | TLSVSPGER |
| | | | WIRQHPGKGLEWIGYIYY | | TCTVSDGSVS | | LSCRTSQSVR | | ATLSCRTSQ |
| | | | SGSTYFNPSLRSRVTMSVD | | RGGYYLTWIR | | SNLAWYQQK | | SVRSNLAW |
| | | | TSKNQFSLKLSSVTAADTA | | QHPGKGLEWI | | PGQAPRLLIY | | YQQKPGQA |
| | | | VYYCARGIAVAPFDYWG | | GYIYYSGSTY | | AASTRATGIP | | PRLLIYAAS |
| | | | QGTLVTVSSASTKGPSVFP | | FNPSLRSRVT | | ARFSGSGSGT | | TRATGIPAR |
| | | | LAPSSKSTSGGTAALGCLV | | MSVDTSKNQ | | EFTLTISSLQS | | FSGSGSGTE |
| | | | KDYFPEPVTVSWNSGALT | | FSLKLSSVTA | | EDFAVYYCQ | | FTLTISSLQS |
| | | | SGVHTFPAVLQSSGLYSLS | | ADTAVYYCA | | QYTNWPFTFG | | EDFAVYYC |
| | | | SVVTVPSSSLGTQTYICNV | | RGIAVAPFDY | | PGTKVDIKRT | | QQYTNWPF |
| | | | NHKPSNTKVDKKVEPKSC | | WGQGTLVTV | | VAAPSVFIFPP | | TFGPGTKVD |
| | | | DKTHTCPPCPAPELLGGPS | | SS | | SDEQLKSGTA | | IK |
| | | | VFLFPPKPKDTLMISRTPE | | | | SVVCLLNNFY | | |
| | | | VTCVVVDVSHEDPEVKFN | | | | PREAKVQWK | | |
| | | | WYVDGVEVHNAKTKPRE | | | | VDNALQSGN | | |
| | | | EQYNSTYRVVSVLTVLHQ | | | | SQESVTEQDS | | |
| | | | DWLNGKEYKCKVSNKAL | | | | KDSTYSLSST | | |
| | | | PAPIEKTISKAKGQPREPQ | | | | LTLSKADYEK | | |
| | | | VYTLPPSRDELTKNQVSL | | | | HKVYACEVT | | |
| | | | WCLVKGFYPSDIAVEWES | | | | HQGLSSPVTK | | |
| | | | NGQPENNYKTTPPVLDSD | | | | SFNRGEC | | |
| | | | GSFFLYSKLTVDKSRWQQ | | | | | | |
| | | | GNVFSCSVMHEALHNHYT | | | | | | |
| | | | QKSLSLSPGK | | | | | | |
| EPI1004 | CEACAM5 | 262 | EVQLVESGGGVVQPGRSL | 263 | EVQLVESGGG | 264 | DIQLTQSPSS | 265 | DIQLTQSPSS |
| | | | RLSCSASGFDFTTYWMSW | | VVQPGRSLRL | | LSASVGDRVT | | LSASVGDR |
| | | | VRQAPGKGLEWIGEIHPDS | | SCSASGFDFT | | ITCKASQDVG | | VTITCKASQ |
| | | | STINYAPSLKDRFTISRDN | | TYWMSWVR | | TSVAWYQQK | | DVGTSVAW |
| | | | AKNTLFLQMDSLRPEDTG | | QAPGKGLEWI | | PGKAPKLLIY | | YQQKPGKA |
| | | | VYFCASLYFGFPWFAYWG | | GEIHPDSSTIN | | WTSTRHTGVP | | PKLLIYWTS |
| | | | QGTPVTVSSASTKGPSVFP | | YAPSLKDRFT | | SRFSGSGSGT | | TRHTGVPSR |
| | | | LAPSSKSTSGGTAALGCLV | | ISRDNAKNTL | | DFTFTISSLQP | | FSGSGSGTD |
| | | | KDYFPEPVTVSWNSGALT | | FLQMDSLRPE | | EDIATYYCQQ | | FTFTISSLQP |
| | | | SGVHTFPAVLQSSGLYSLS | | DTGVYFCASL | | YSLYRSFGQG | | EDIATYYCQ |
| | | | SVVTVPSSSLGTQTYICNV | | YFGFPWFAY | | TKVEIKRTVA | | QYSLYRSFG |
| | | | NHKPSNTKVDKRVEPKSC | | WGQGTPVTV | | APSVFIFPPSD | | QGTK VEIK |
| | | | DKTHTCPPCPAPELLGGPS | | SS | | EQLKSGTASV | | |
| | | | VFLFPPKPKDTLMISRTPE | | | | VCLLNNFYPR | | |
| | | | VTCVVVDVSHEDPEVKFN | | | | EAKVQWKVD | | |
| | | | WYVDGVEVHNAKTKPRE | | | | NALQSGNSQE | | |
| | | | EQYNSTYRVVSVLTVLHQ | | | | SVTEQDSKDS | | |
| | | | DWLNGKEYKCKVSNKAL | | | | TYSLSSTLTLS | | |
| | | | PAPIEKTISKAKGQPREPQ | | | | KADYEKHKV | | |
| | | | VYTLPPSRDELTKNQVSL | | | | YACEVTHQG | | |
| | | | WCLVKGFYPSDIAVEWES | | | | LSSPVTKSFN | | |
| | | | NGQPENNYKTTPPVLDSD | | | | RGEC | | |
| | | | GSFFLYSKLTVDKSR WQQ | | | | | | |
| | | | GNVFSCSVMHEALHNHYT | | | | | | |
| | | | QKSLSLSPGK | | | | | | |
| EPI1006 | CEACAM5 | 270 | EVQLQESGPGLVKPGGSL | 271 | EVQLQESGPG | 272 | DIQMTQSPA | 273 | DIQMTQSPA |
| | | | SLSCAASGFVFSSYDMSW | | LVKPGGSLSL | | SLSASVGDRV | | SLSASVGDR |
| | | | VRQTPERGLEWVAYISSG | | SCAASGFVFS | | TITCRASENIF | | VTITCRASE |
| | | | GGITYAPSTVKGRFTVSRD | | SYDMSWVRQ | | SYLAWYQQK | | NIFSYLAWY |
| | | | NAKNTLYLQMNSLTSEDT | | TPERGLEWV | | PGKSPKLLVY | | QQKPGKSP |
| | | | AVYYCAAHYFGSSGPFAY | | AYISSGGGITY | | NTRTLAEGVP | | KLLVYNTRT |
| | | | WGQGTLVTVSSASTKGPS | | APSTVKGRFT | | SRFSGSGSGT | | LAEGVPSRF |
| | | | VFPLAPSSKSTSGGTAALG | | VSRDNAKNT | | DFSLTISSLQP | | SGSGSGTDF |
| | | | CLVKDYFPEPVTVSWNSG | | LYLQMNSLTS | | EDFATYYCQ | | SLTISSLQPE |
| | | | ALTSGVHTFPAVLQSSGLY | | EDTAVYYCA | | HHYGTPFTFG | | DFATYYCQ |
| | | | SLSSVVTVPSSSLGTQTYIC | | AHYFGSSGPF | | SGTKLEIKRT | | HHYGTPFTF |
| | | | NVNHKPSNTKVDKKVEPK | | AYWGQGTLV | | VAAPSVFIFPP | | GSGTKLEIK |
| | | | SCDKTHTCPPCPAPELLGG | | TVSS | | SDEQLKSGTA | | |
| | | | PSVFLFPPKPKDTLMISRTP | | | | SVVCLLNNFY | | |
| | | | EVTCVVVDVSHEDPEVKF | | | | PREAKVQWK | | |
| | | | NWYVDGVEVHNAKTKPR | | | | VDNALQSGN | | |
| | | | EEQYNSTYRVVSVLTVLH | | | | SQESVTEQDS | | |
| | | | QDWLNGKEYKCKVSNKA | | | | KDSTYSLSST | | |
| | | | LPAPIEKTISKAKGQPREPQ | | | | LTLSKADYEK | | |
| | | | VYTLPPSRDELTKNQVSL | | | | HKVYACEVT | | |
| | | | WCLVKGFYPSDIAVEWES | | | | HQGLSSPVTK | | |
| | | | NGQPENNYKTTPPVLDSD | | | | SFNRGEC | | |
| | | | GSFFLYSKLTVDKSRWQQ | | | | | | |
| | | | GNVFSCSVMHEALHNHYT | | | | | | |
| | | | QKSLSLSPGK | | | | | | |
| EPI1015 | CD71 | 302 | QVQLVQSGAEVKKPGAS | 303 | QVQLVQSGA | 304 | DIQMTQSPS | 305 | DIQMTQSPS |
| | | | VKMSCKASGYTFTSYWM | | EVKKPGASV | | SLSASVGDRV | | SLSASVGDR |
| | | | HWVRQAPGQGLEWIGAIY | | KMSCKASGY | | TITCSASSSVY | | VTITCSASSS |
| | | | PGNSETGYAQKFQGRATL | | TFTSYWMHW | | YMYWFQQKP | | VYYMYWF |
| | | | TADTSTSTAYMELSSLRSE | | VRQAPGQGL | | GKAPKLWIYS | | QQKPGKAP |
| | | | DTAVYYCTRENWDPGFAF | | EWIGAIYPGN | | TSNLASGVPS | | KLWIYSTSN |
| | | | WGQGTLITVSSASTKGPSV | | SETGYAQKFQ | | RFSGSGSGTD | | LASGVPSRF |
| | | | FPLAPSSKSTSGGTAALGC | | GRATLTADTS | | YTLTISSMQP | | SGSGSGTDY |
| | | | LVKDYFPEPVTVSWNSGA | | TSTAYMELSS | | EDFATYYCQ | | TLTISSMQP |
| | | | LTSGVHTFPAVLQSSGLYS | | LRSEDTAVYY | | QRRNYPYTFG | | EDFATYYC |
| | | | LSSVVTVPSSSLGTQTYIC | | CTRENWDPG | | QGTKLEIKRT | | QQRRNYPY |
| | | | NVNHKPSNTKVDKKVEPK | | FAFWGQGTLI | | VAAPSVFIFPP | | TFGQGTKLE |
| | | | SCDKTHTCPPCPAPELLGG | | TVSS | | SDEQLKSGTA | | IK |
| | | | PSVFLFPPKPKDTLMISRTP | | | | SVVCLLNNFY | | |
| | | | EVTCVVVDVSHEDPEVKF | | | | PREAKVQWK | | |
| | | | NWYVDGVEVHNAKTKPR | | | | VDNALQSGN | | |
| | | | EEQYNSTYRVVSVLTVLH | | | | SQESVTEQDS | | |
| | | | QDWLNGKEYKCKVSNKA | | | | KDSTYSLSST | | |
| | | | LPAPIEKTISKAKGQPREPQ | | | | LTLSKADYEK | | |
| | | | VYTLPPSRDELTKNQVSL | | | | HKVYACEVT | | |
| | | | WCLVKGFYPSDIAVEWES | | | | HQGLSSPVTK | | |
| | | | NGQPENNYKTTPPVLDSD | | | | SFNRGEC | | |
| | | | GSFFLYSKLTVDKSRWQQ | | | | | | |
| | | | GNVFSCSVMHEALHNHYT | | | | | | |
| | | | QKSLSLSPGK | | | | | | |
| EPI867 | CD71 | 218 | QVQLQQSGPDLVKPGASV | 219 | QVQLQQSGP | 220 | DILLTQSPAI | 221 | DILLTQSPAI |
| | | | RISCKASGYTFAGHYVHW | | DLVKPGASVR | | LSVSPGDRVS | | LSVSPGDRV |
| | | | VKQRPGRGLEWIGWIFPG | | ISCKASGYTF | | FSCRASQSIGT | | SFSCRASQSI |
| | | | KVNTKYNEKFKGKATLTA | | AGHYVHWVK | | SIHWYQQRTD | | GTSIHWYQ |
| | | | DKSSSTAYMQLSSLTSEDS | | QRPGRGLEWI | | GSPRLLIKYA | | QRTDGSPRL |
| | | | AVYFCARVGYDYPYYFD | | GWIFPGKVNT | | SESISGIPSRFS | | LIKYASESIS |
| | | | YWGQGTTLTVSSASTKGP | | KYNEKFKGK | | GSGSGTDFTL | | GIPSRFSGSG |
| | | | SVFPLAPSSKSTSGGTAAL | | ATLTADKSSS | | SINSVESEDV | | SGTDFTLSI |
| | | | GCLVKDYFPEPVTVSWNS | | TAYMQLSSLT | | ADYYCQQSSS | | NSVESEDVA |
| | | | GALTSGVHTFPAVLQSSGL | | SEDSAVYFCA | | WPFTFGSGTK | | DYYCQQSSS |
| | | | YSLSSVVTVPSSSLGTQTYI | | RVGYDYPYY | | LEIKRTVAAP | | WPFTFGSGT |
| | | | CNVNHKPSNTKVDKKVEP | | FDYWGQGTT | | SVFIFPPSDEQ | | KLEIK |
| | | | KSCDKTHTCPPCPAPELLG | | LTVSS | | LKSGTASVVC | | |
| | | | GPSVFLFPPKPKDTLMISR | | | | LLNNFYPREA | | |
| | | | TPEVTCVVVDVSHEDPEV | | | | KVQWKVDNA | | |
| | | | KFNWYVDGVEVHNAKTK | | | | LQSGNSQESV | | |
| | | | PREEQYNSTYRVVSVLTV | | | | TEQDSKDSTY | | |
| | | | LHQDWLNGKEYKCKVSN | | | | SLSSTLTLSKA | | |
| | | | KALPAPIEKTISKAKGQPR | | | | DYEKHKVYA | | |
| | | | EPQVYTLPPSRDELTKNQV | | | | CEVTHQGLSS | | |
| | | | SLWCLVKGFYPSDIAVEW | | | | PVTKSFNRGE | | |
| | | | ESNGQPENNYKTTPPVLDS | | | | C | | |
| | | | DGSFFLYSKLTVDKSRWQ | | | | | | |
| | | | QGNVFSCSVMHEALHNH | | | | | | |
| | | | YTQKSLSLSPGK | | | | | | |
| EPI873 | CD71 | 242 | EVQLVQSGAEVKKPGASV | 243 | EVQLVQSGAE | 244 | DIQMTQSPS | 245 | DIQMTQSPS |
| | | | KVSCKASGYTFTSYWMH | | VKKPGASVK | | SLSASVGDRV | | SLSASVGDR |
| | | | WVRQAPGQRLEWIGEINP | | VSCKASGYTF | | TITCRASDNL | | VTITCRASD |
| | | | TNGRTNYIEKFKSRATLTV | | TSYWMHWV | | YSNLAWYQQ | | NLYSNLAW |
| | | | DKSASTAYMELSSLRSEDT | | RQAPGQRLE | | KPGKSPKLLV | | YQQKPGKS |
| | | | AVYYCARGTRAYHYWGQ | | WIGEINPTNG | | YDATNLADG | | PKLLVYDAT |
| | | | GTMVTVSSASTKGPSVFPL | | RTNYIEKFKS | | VPSRFSGSGS | | NLADGVPS |
| | | | APSSKSTSGGTAALGCLV | | RATLTVDKSA | | GTDYTLTISSL | | RFSGSGSGT |
| | | | KDYFPEPVTVSWNSGALT | | STAYMELSSL | | QPEDFATYYC | | DYTLTISSL |
| | | | SGVHTFPAVLQSSGLYSLS | | RSEDTAVYYC | | QHFWGTPLTF | | QPEDFATYY |
| | | | SVVTVPSSSLGTQTYICNV | | ARGTRAYHY | | GQGTKVEIKR | | CQHFWGTP |
| | | | NHKPSNTKVDKKVEPKSC | | WGQGTMVTV | | TVAAPSVFIFP | | LTFGQGTKV |
| | | | DKTHTCPPCPAPELLGGPS | | SS | | PSDEQLKSGT | | EIK |
| | | | VFLFPPKPKDTLMISRTPE | | | | ASVVCLLNNF | | |
| | | | VTCVVVDVSHEDPEVKFN | | | | YPREAKVQW | | |
| | | | WYVDGVEVHNAKTKPRE | | | | KVDNALQSG | | |
| | | | EQYNSTYRVVSVLTVLHQ | | | | NSQESVTEQD | | |
| | | | DWLNGKEYKCKVSNKAL | | | | SKDSTYSLSS | | |
| | | | PAPIEKTISKAKGQPREPQ | | | | TLTLSKADYE | | |
| | | | VYTLPPSRDELTKNQVSL | | | | KHKVYACEV | | |
| | | | WCLVKGFYPSDIAVEWES | | | | THQGLSSPVT | | |
| | | | NGQPENNYKTTPPVLDSD | | | | KSFNRGEC | | |
| | | | GSFFLYSKLTVDKSRWQQ | | | | | | |
| | | | GNVFSCSVMHEALHNHYT | | | | | | |
| | | | QKSLSLSPGK | | | | | | |
| EPI874 | CD71 | 246 | EVQLVQSGAEVKKPGASV | 247 | EVQLVQSGAE | 248 | DIQMTQSPS | 249 | DIQMTQSPS |
| | | | KVSCKASGYTFTSYWMH | | VKKPGASVK | | SLSASVGDRV | | SLSASVGDR |
| | | | WVRQAPGQRLEWIGEIAP | | VSCKASGYTF | | TITCRASDNL | | VTITCRASD |
| | | | TNGRTNYIEKFKSRATLTV | | TSYWMHWV | | YSNLAWYQQ | | NLYSNLAW |
| | | | DKSASTAYMELSSLRSEDT | | RQAPGQRLE | | KPGKSPKLLV | | YQQKPGKS |
| | | | AVYYCARGTRAYHYWGQ | | WIGEIAPTNG | | YDATNLADG | | PKLLVYDAT |
| | | | GTMVTVSSASTKGPSVFPL | | RTNYIEKFKS | | VPSRFSGSGS | | NLADGVPS |
| | | | APSSKSTSGGTAALGCLV | | RATLTVDKSA | | GTDYTLTISSL | | RFSGSGSGT |
| | | | KDYFPEPVTVSWNSGALT | | STAYMELSSL | | QPEDFATYYC | | DYTLTISSL |
| | | | SGVHTFPAVLQSSGLYSLS | | RSEDTAVYYC | | QHFWGTPLTF | | QPEDFATYY |
| | | | SVVTVPSSSLGTQTYICNV | | ARGTRAYHY | | GQGTKVEIKR | | CQHFWGTP |
| | | | NHKPSNTKVDKKVEPKSC | | WGQGTMVTV | | TVAAPSVFIFP | | LTFGQGTKV |
| | | | DKTHTCPPCPAPELLGGPS | | SS | | PSDEQLKSGT | | EIK |
| | | | VFLFPPKPKDTLMISRTPE | | | | ASVVCLLNNF | | |
| | | | VTCVVVDVSHEDPEVKFN | | | | YPREAKVQW | | |
| | | | WYVDGVEVHNAKTKPRE | | | | KVDNALQSG | | |
| | | | EQYNSTYRVVSVLTVLHQ | | | | NSQESVTEQD | | |
| | | | DWLNGKEYKCKVSNKAL | | | | SKDSTYSLSS | | |
| | | | PAPIEKTISKAKGQPREPQ | | | | TLTLSKADYE | | |
| | | | VYTLPPSRDELTKNQVSL | | | | KHKVYACEV | | |
| | | | WCLVKGFYPSDIAVEWES | | | | THQGLSSPVT | | |
| | | | NGQPENNYKTTPPVLDSD | | | | KSFNRGEC | | |
| | | | GSFFLYSKLTVDKSRWQQ | | | | | | |
| | | | GNVFSCSVMHEALHNHYT | | | | | | |
| | | | QKSLSLSPGK | | | | | | |
| EPI875 | CD71 | 250 | EVQLVQSGAEVKKPGASV | 251 | EVQLVQSGAE | 252 | DIQMTQSPS | 253 | DIQMTQSPS |
| | | | KVSCKASGYTFTSYWMH | | VKKPGASVK | | SLSASVGDRV | | SLSASVGDR |
| | | | WVRQAPGQRLEWIGEINP | | VSCKASGYTF | | TITCRASDNL | | VTITCRASD |
| | | | ANGRTNYIEKFKSRATLTV | | TSYWMHWV | | YSNLAWYQQ | | NLYSNLAW |
| | | | DKSASTAYMELSSLRSEDT | | RQAPGQRLE | | KPGKSPKLLV | | YQQKPGKS |
| | | | AVYYCARGTRAYHYWGQ | | WIGEINPANG | | YDATNLADG | | PKLLVYDAT |
| | | | GTMVTVSSASTKGPSVFPL | | RTNYIEKFKS | | VPSRFSGSGS | | NLADGVPS |
| | | | APSSKSTSGGTAALGCLV | | RATLTVDKSA | | GTDYTLTISSL | | RFSGSGSGT |
| | | | KDYFPEPVTVSWNSGALT | | STAYMELSSL | | QPEDFATYYC | | DYTLTISSL |
| | | | SGVHTFPAVLQSSGLYSLS | | RSEDTAVYYC | | QHFWGTPLTF | | QPEDFATYY |
| | | | SVVTVPSSSLGTQTYICNV | | ARGTRAYHY | | GQGTKVEIKR | | CQHFWGTP |
| | | | NHKPSNTKVDKKVEPKSC | | WGQGTMVTV | | TVAAPSVFIFP | | LTFGQGTKV |
| | | | DKTHTCPPCPAPELLGGPS | | SS | | PSDEQLKSGT | | EIK |
| | | | VFLFPPKPKDTLMISRTPE | | | | ASVVCLLNNF | | |
| | | | VTCVVVDVSHEDPEVKFN | | | | YPREAKVQW | | |
| | | | WYVDGVEVHNAKTKPRE | | | | KVDNALQSG | | |
| | | | EQYNSTYRVVSVLTVLHQ | | | | NSQESVTEQD | | |
| | | | DWLNGKEYKCKVSNKAL | | | | SKDSTYSLSS | | |
| | | | PAPIEKTISKAKGQPREPQ | | | | TLTLSKADYE | | |
| | | | VYTLPPSRDELTKNQVSL | | | | KHKVYACEV | | |
| | | | WCLVKGFYPSDIAVEWES | | | | THQGLSSPVT | | |
| | | | NGQPENNYKTTPPVLDSD | | | | KSFNRGEC | | |
| | | | GSFFLYSKLTVDKSRWQQ | | | | | | |
| | | | GNVFSCSVMHEALHNHYT | | | | | | |
| | | | QKSLSLSPGK | | | | | | |
| EPI876 | CD71 | 254 | EVQLVQSGAEVKKPGASV | 255 | EVQLVQSGAE | 256 | DIQMTQSPS | 257 | DIQMTQSPS |
| | | | KVSCKASGYTFTSYWMH | | VKKPGASVK | | SLSASVGDRV | | SLSASVGDR |
| | | | WVRQAPGQRLEWIGEINP | | VSCKASGYTF | | TITCRASDNL | | VTITCRASD |
| | | | ANGRTNYIEKFKSRATLTV | | TSYWMHWV | | YSNLAWYQQ | | NLYSNLAW |
| | | | DKSASTAYMELSSLRSEDT | | RQAPGQRLE | | KPGKSPKLLV | | YQQKPGKS |
| | | | AVYYCARGTRAYHYWGQ | | WIGEINPANG | | YDATNLADG | | PKLLVYDAT |
| | | | GTMVTVSSASTKGPSVFPL | | RTNYIEKFKS | | VPSRFSGSGS | | NLADGVPS |
| | | | APSSKSTSGGTAALGCLV | | RATLTVDKSA | | GTDYTLTISSL | | RFSGSGSGT |
| | | | KDYFPEPVTVSWNSGALT | | STAYMELSSL | | QPEDFATYYC | | DYTLTISSL |
| | | | SGVHTFPAVLQSSGLYSLS | | RSEDTAVYYC | | QHFAGTPLTF | | QPEDFATYY |
| | | | SVVTVPSSSLGTQTYICNV | | ARGTRAYHY | | GQGTKVEIKR | | CQHFAGTPL |
| | | | NHKPSNTKVDKKVEPKSC | | WGQGTMVTV | | TVAAPSVFIFP | | TFGQGTKVE |
| | | | DKTHTCPPCPAPELLGGPS | | SS | | PSDEQLKSGT | | IK |
| | | | VFLFPPKPKDTLMISRTPE | | | | ASVVCLLNNF | | |
| | | | VTCVVVDVSHEDPEVKFN | | | | YPREAKVQW | | |
| | | | WYVDGVEVHNAKTKPRE | | | | KVDNALQSG | | |
| | | | EQYNSTYRVVSVLTVLHQ | | | | NSQESVTEQD | | |
| | | | DWLNGKEYKCKVSNKAL | | | | SKDSTYSLSS | | |
| | | | PAPIEKTISKAKGQPREPQ | | | | TLTLSKADYE | | |
| | | | VYTLPPSRDELTKNQVSL | | | | KHKVYACEV | | |
| | | | WCLVKGFYPSDIAVEWES | | | | THQGLSSPVT | | |
| | | | NGQPENNYKTTPPVLDSD | | | | KSFNRGEC | | |
| | | | GSFFLYSKLTVDKSRWQQ | | | | | | |
| | | | GNVFSCSVMHEALHNHYT | | | | | | |
| | | | QKSLSLSPGK | | | | | | |
| EPI 1094 | CD71 | 314 | EVQLVQSGAEVKKPGASV | 315 | EVQLVQSGAE | 316 | DIQMTQSPS | 317 | DIQMTQSPS |
| | | | KVSCKASGYTFTSYWMH | | VKKPGASVK | | SLSASVGDRV | | SLSASVGDR |
| | | | WVRQAPGQRLEWIGEINP | | VSCKASGYTF | | TITCRASDNL | | VTITCRASD |
| | | | TNGRTNYIEKFKSRATLTV | | TSYWMHWV | | YSNLAWYQQ | | NLYSNLAW |
| | | | DKSASTAYMELSSLRSEDT | | RQAPGQRLE | | KPGKSPKLLV | | YQQKPGKS |
| | | | AVYYCARGTRAYHYWGQ | | WIGEINPTNG | | YDATNLADG | | PKLLVYDAT |
| | | | GTMVTVSSASTKGPSVFPL | | RTNYIEKFKS | | VPSRFSGSGS | | NLADGVPS |
| | | | APSSKSTSGGTAALGCLV | | RATLTVDKSA | | GTDYTLTISSL | | RFSGSGSGT |
| | | | KDYFPEPVTVSWNSGALT | | STAYMELSSL | | QPEDFATYYC | | DYTLTISSL |
| | | | SGVHTFPAVLQSSGLYSLS | | RSEDTAVYYC | | QHFAGTPLTF | | QPEDFATYY |
| | | | SVVTVPSSSLGTQTYICNV | | ARGTRAYHY | | GQGTKVEIKR | | CQHFAGTPL |
| | | | NHKPSNTKVDKKVEPKSC | | WGQGTMVTV | | TVAAPSVFIFP | | TFGQGTKVE |
| | | | DKTHTCPPCPAPELLGGPS | | SS | | PSDEQLKSGT | | IK |
| | | | VFLFPPKPKDTLMISRTPE | | | | ASVVCLLNNF | | |
| | | | VTCVVVDVSHEDPEVKFN | | | | YPREAKVQW | | |
| | | | WYVDGVEVHNAKTKPRE | | | | KVDNALQSG | | |
| | | | EQYNSTYRVVSVLTVLHQ | | | | NSQESVTEQD | | |
| | | | DWLNGKEYKCKVSNKAL | | | | SKDSTYSLSS | | |
| | | | PAPIEKTISKAKGQPREPQ | | | | TLTLSKADYE | | |
| | | | VYTLPPSRDELTKNQVSL | | | | KHKVYACEV | | |
| | | | WCLVKGFYPSDIAVEWES | | | | THQGLSSPVT | | |
| | | | NGQPENNYKTTPPVLDSD | | | | KSFNRGEC | | |
| | | | GSFFLYSKLTVDKSRWQQ | | | | | | |
| | | | GNVFSCSVMHEALHNHYT | | | | | | |
| | | | QKSLSLSPGK | | | | | | |
| EPI1095 | CD71 | 318 | EVQLVQSGAEVKKPGASV | 319 | EVQLVQSGAE | 320 | DIQMTQSPS | 321 | DIQMTQSPS |
| | | | KVSCKASGYTFTSYWMH | | VKKPGASVK | | SLSASVGDRV | | SLSASVGDR |
| | | | WVRQAPGQRLEWIGEIAP | | VSCKASGYTF | | TITCRASDNL | | VTITCRASD |
| | | | TNGRTNYIEKFKSRATLTV | | TSYWMHWV | | YSNLAWYQQ | | NLYSNLAW |
| | | | DKSASTAYMELSSLRSEDT | | RQAPGQRLE | | KPGKSPKLLV | | YQQKPGKS |
| | | | AVYYCARGTRAYHYWGQ | | WIGEIAPTNG | | YDATNLADG | | PKLLVYDAT |
| | | | GTMVTVSSASTKGPSVFPL | | RTNYIEKFKS | | VPSRFSGSGS | | NLADGVPS |
| | | | APSSKSTSGGTAALGCLV | | RATLTVDKSA | | GTDYTLTISSL | | RFSGSGSGT |
| | | | KDYFPEPVTVSWNSGALT | | STAYMELSSL | | QPEDFATYYC | | DYTLTISSL |
| | | | SGVHTFPAVLQSSGLYSLS | | RSEDTAVYYC | | QHFAGTPLTF | | QPEDFATYY |
| | | | SVVTVPSSSLGTQTYICNV | | ARGTRAYHY | | GQGTKVEIKR | | CQHFAGTPL |
| | | | NHKPSNTKVDKKVEPKSC | | WGQGTMVTV | | TVAAPSVFIFP | | TFGQGTKVE |
| | | | DKTHTCPPCPAPELLGGPS | | SS | | PSDEQLKSGT | | IK |
| | | | VFLFPPKPKDTLMISRTPE | | | | ASVVCLLNNF | | |
| | | | VTCVVVDVSHEDPEVKFN | | | | YPREAKVQW | | |
| | | | WYVDGVEVHNAKTKPRE | | | | KVDNALQSG | | |
| | | | EQYNSTYRVVSVLTVLHQ | | | | NSQESVTEQD | | |
| | | | DWLNGKEYKCKVSNKAL | | | | SKDSTYSLSS | | |
| | | | PAPIEKTISKAKGQPREPQ | | | | TLTLSKADYE | | |
| | | | VYTLPPSRDELTKNQVSL | | | | KHKVYACEV | | |
| | | | WCLVKGFYPSDIAVEWES | | | | THQGLSSPVT | | |
| | | | INGQPENNYKTTPPVLDSD | | | | KSFNRGEC | | |
| | | | GSFFLYSKLTVDKSRWQQ | | | | | | |
| | | | GNVFSCSVMHEALHNHYT | | | | | | |
| | | | QKSLSLSPGK | | | | | | |
| EPI809 | RNF43 | 18 | QVQLQESGPGLVKPSETLS | 19 | QVQLQESGPG | 20 | DIQMTQSPS | 21 | DIQMTQSPS |
| | | | LTCTVSGGSISSSNYYWG | | LVKPSETLSL | | SLSASVGDRV | | SLSASVGDR |
| | | | WIRQPPGKGLEWIGNIYYR | | TCTVSGGSISS | | TITCRASQSIS | | VTITCRASQ |
| | | | GYTYYNPSLKSRVTISVDT | | SNYYWGWIR | | SYLNWYQQK | | SISSYLNWY |
| | | | SKKQFSLTLSSVTAADTA | | QPPGKGLEWI | | PGKAPKLLIY | | QQKPGKAP |
| | | | MYYCAREGSDYGDYVGA | | GNIYYRGYTY | | AASSLQSGVP | | KLLIYAASS |
| | | | FDIWDQGTMVTVSSASTK | | YNPSLKSRVT | | SRFSGSGSGT | | LQSGVPSRF |
| | | | GPSVFPLAPSSKSTSGGTA | | ISVDTSKKQF | | DFTLTISSLQP | | SGSGSGTDF |
| | | | ALGCLVKDYFPEPVTVSW | | SLTLSSVTAA | | EDFATYYCQ | | TLTISSLQPE |
| | | | NSGALTSGVHTFPAVLQSS | | DTAMYYCAR | | QSYSTPPTFG | | DFATYYCQ |
| | | | GLYSLSSVVTVPSSSLGTQ | | EGSDYGDYV | | QGTKVEIKRT | | QSYSTPPTF |
| | | | TYICNVNHKPSNTKVDKK | | GAFDIWDQG | | VAAPSVFIFPP | | GQGTKVEIK |
| | | | VEPKSCDKTHTCPPCPAPE | | TMVTVSS | | SDEQLKSGTA | | |
| | | | LLGGPSVFLFPPKPKDTLM | | | | SVVCLLNNFY | | |
| | | | ISRTPEVTCVVVDVSHEDP | | | | PREAKVQWK | | |
| | | | EVKFNWYVDGVEVHNAK | | | | VDNALQSGN | | |
| | | | TKPREEQYNSTYRVVSVL | | | | SQESVTEQDS | | |
| | | | TVLHQDWLNGKEYKCKV | | | | KDSTYSLSST | | |
| | | | SNKALPAPIEKTISKAKGQ | | | | LTLSKADYEK | | |
| | | | PREPQVYTLPPSRDELTKN | | | | HKVYACEVT | | |
| | | | QVSLWCLVKGFYPSDIAV | | | | HQGLSSPVTK | | |
| | | | EWESNGQPENNYKTTPPV | | | | SFNRGEC | | |
| | | | LDSDGSFFLYSKLTVDKSR | | | | | | |
| | | | WQQGNVFSCSVMHEALH | | | | | | |
| | | | NHYTQKSLSLSPGK | | | | | | |
| EPI810 | RNF43 | 22 | EVQLVQSGAEVKKPGASV | 23 | EVQLVQSGAE | 24 | EIVMTQSPA | 25 | EIVMTQSPA |
| | | | KVSCKASGYTFTTYTIHW | | VKKPGASVK | | TLSVSPGERA | | TLSVSPGER |
| | | | VRQAPGQGLEWMGYINPR | | VSCKASGYTF | | TLSCKASQNV | | ATLSCKASQ |
| | | | SGYTEYNQKFQDRVTMTR | | TTYTIHWVRQ | | GINVAWYQQ | | NVGINVAW |
| | | | DTSTSTVYMELSSLRSEDT | | APGQGLEWM | | KPGQAPRALI | | YQQKPGQA |
| | | | AVYYCARSYEFWGQGTT | | GYINPRSGYT | | YSASYRYSGI | | PRALIYSAS |
| | | | VTVSSASTKGPSVFPLAPS | | EYNQKFQDR | | PARFSGSGSG | | YRYSGIPAR |
| | | | SKSTSGGTAALGCLVKDY | | VTMTRDTSTS | | TEFTLTISSLQ | | FSGSGSGTE |
| | | | FPEPVTVSWNSGALTSGV | | TVYMELSSLR | | SEDFAVYYCH | | FTLTISSLQS |
| | | | HTFPAVLQSSGLYSLSSVV | | SEDTAVYYC | | QYKTYPYTFG | | EDFAVYYC |
| | | | TVPSSSLGTQTYICNVNHK | | ARSYEFWGQ | | GGTKLEIKRT | | HQYKTYPY |
| | | | PSNTKVDKKVEPKSCDKT | | GTTVTVSS | | VAAPSVFIFPP | | TFGGGTKLE |
| | | | HTCPPCPAPELLGGPSVFL | | | | SDEQLKSGTA | | IK |
| | | | FPPKPKDTLMISRTPEVTC | | | | SVVCLLNNFY | | |
| | | | VVVDVSHEDPEVKFNWY | | | | PREAKVQWK | | |
| | | | VDGVEVHNAKTKPREEQY | | | | VDNALQSGN | | |
| | | | NSTYRVVSVLTVLHQDWL | | | | SQESVTEQDS | | |
| | | | NGKEYKCKVSNKALPAPI | | | | KDSTYSLSST | | |
| | | | EKTISKAKGQPREPQVYTL | | | | LTLSKADYEK | | |
| | | | PPSRDELTKNQVSLWCLV | | | | HKVYACEVT | | |
| | | | KGFYPSDIAVEWESNGQP | | | | HQGLSSPVTK | | |
| | | | ENNYKTTPPVLDSDGSFFL | | | | SFNRGEC | | |
| | | | YSKLTVDKSRWQQGNVFS | | | | | | |
| | | | CSVMHEALHNHYTQKSLS | | | | | | |
| | | | LSPGK | | | | | | |
| EPI811 | RNF43 | 26 | AVQLVESGGGSVQPGRSM | 27 | AVQLVESGG | 28 | DVVLTQTPV | 29 | DVVLTQTPV |
| | | | RLSCAASGFTFSNYDMTW | | GSVQPGRSM | | SLSVTVGDQA | | SLSVTVGDQ |
| | | | VRQAPTKGLEWVASITSD | | RLSCAASGFT | | SISCRSSQSLE | | ASISCRSSQS |
| | | | GGSTYSRDSVKGRFTISRD | | FSNYDMTWV | | YSDGYSYLE | | LEYSDGYSY |
| | | | NAKSTLYLQMDSLRSEDT | | RQAPTKGLE | | WYLQKPGQS | | LEWYLQKP |
| | | | ATYYCTTDRGRYLPYYFD | | WVASITSDGG | | PQLLIYEVSSR | | GQSPQLLIY |
| | | | YWGQGVMVTVSSASTK | | STYSRDSVKG | | FSGVPDRFIGS | | EVSSRFSGV |
| | | | PSVFPLAPSSKSTSGGTAA | | RFTISRDNAK | | GSGTDFTLKIS | | PDRFIGSGS |
| | | | LGCLVKDYFPEPVTVSWN | | STLYLQMDSL | | RVEPEDLGVY | | GTDFTLKIS |
| | | | SGALTSGVHTFPAVLQSSG | | RSEDTATYYC | | YCFQAIHDPT | | RVEPEDLGV |
| | | | LYSLSSVVTVPSSSLGTQT | | TTDRGRYLPY | | FGAGTKLELK | | YYCFQAIHD |
| | | | YICNVNHKPSNTKVDKKV | | YFDYWGQGV | | RTVAAPSVFIF | | PTFGAGTKL |
| | | | EPKSCDKTHTCPPCPAPEL | | MVTVSS | | PPSDEQLKSG | | ELK |
| | | | LGGPSVFLFPPKPKDTLMI | | | | TASVVCLLNN | | |
| | | | SRTPEVTCVVVDVSHEDP | | | | FYPREAKVQ | | |
| | | | EVKFNWYVDGVEVHNAK | | | | WKVDNALQS | | |
| | | | TKPREEQYNSTYRVVSVL | | | | GNSQESVTEQ | | |
| | | | TVLHQDWLNGKEYKCKV | | | | DSKDSTYSLS | | |
| | | | SNKALPAPIEKTISKAKGQ | | | | STLTLSKADY | | |
| | | | PREPQVYTLPPSRDELTKN | | | | EKHKVYACE | | |
| | | | QVSLWCLVKGFYPSDIAV | | | | VTHQGLSSPV | | |
| | | | EWESNGQPENNYKTTPPV | | | | TKSFNRGEC | | |
| | | | LDSDGSFFLYSKLTVDKSR | | | | | | |
| | | | WQQGNVFSCSVMHEALH | | | | | | |
| | | | NHYTQKSLSLSPGK | | | | | | |
| EPI860 | RNF43 | 198 | QVQLVQSGAEVKKPGAS | 199 | QVQLVQSGA | 200 | DIVMTQSPD | 201 | DIVMTQSPD |
| | | | VKVSCKASGFNIKDTYIH | | EVKKPGASV | | SLAVSLGERA | | SLAVSLGER |
| | | | WVRQAPGQGLEWMGRID | | KVSCKASGFN | | TINCRASESV | | ATINCRASE |
| | | | PANGKANYDPKFQGRVT | | IKDTYIHWVR | | DSYGNSFMH | | SVDSYGNSF |
| | | | MTRDTSTSTVYMELSSLRS | | QAPGQGLEW | | WYQQKPGQP | | MHWYQQK |
| | | | EDTAVYYCALGGGYYGM | | MGRIDPANG | | PKLLIYLASN | | PGQPPKLLI |
| | | | DYWGQGTLVTVSSASTKG | | KANYDPKFQ | | LESGVPDRFS | | YLASNLESG |
| | | | PSVFPLAPSSKSTSGGTAA | | GRVTMTRDT | | GSGSGTDFTL | | VPDRFSGSG |
| | | | LGCLVKDYFPEPVTVSWN | | STSTVYMELS | | TISSLQAEDV | | SGTDFTLTIS |
| | | | SGALTSGVHTFPAVLQSSG | | SLRSEDTAVY | | AVYYCQQNN | | SLQAEDVA |
| | | | LYSLSSVVTVPSSSLGTQT | | YCALGGGYY | | EDPLTFGQGT | | VYYCQQNN |
| | | | YICNVNHKPSNTKVDKKV | | GMDYWGQG | | KVEIKRTVAA | | EDPLTFGQG |
| | | | EPKSCDKTHTCPPCPAPEL | | TLVTVSS | | PSVFIFPPSDE | | TKVEIK |
| | | | LGGPSVFLFPPKPKDTLMI | | | | QLKSGTASVV | | |
| | | | SRTPEVTCVVVDVSHEDP | | | | CLLNNFYPRE | | |
| | | | EVKFNWYVDGVEVHNAK | | | | AKVQWKVDN | | |
| | | | TKPREEQYNSTYRVVSVL | | | | ALQSGNSQES | | |
| | | | TVLHQDWLNGKEYKCKV | | | | VTEQDSKDST | | |
| | | | SNKALPAPIEKTISKAKGQ | | | | YSLSSTLTLSK | | |
| | | | PREPQVYTLPPSRDELTKN | | | | ADYEKHKVY | | |
| | | | QVSLWCLVKGFYPSDIAV | | | | ACEVTHQGLS | | |
| | | | EWESNGQPENNYKTTPPV | | | | SPVTKSFNRG | | |
| | | | LDSDGSFFLYSKLTVDKSR | | | | EC | | |
| | | | WQQGNVFSCSVMHEALH | | | | | | |
| | | | NHYTQKSLSLSPGK | | | | | | |
| EPI861 | RNF43 | 202 | QQQLEEYGGDLVQPEGSL | 203 | QQQLEEYGG | 204 | AEIVMTQTP | 205 | AEIVMTQTP |
| | | | TLTCKASGLDFSSSYWMC | | DLVQPEGSLT | | SSKSAAVGDT | | SSKSAAVG |
| | | | WVRQAPGKGLEWIACIYT | | LTCKASGLDF | | VTIKCQASQSI | | DTVTIKCQA |
| | | | GSSGSTSYASWAKGRFTIS | | SSSYWMCWV | | TSYLSWYQQ | | SQSITSYLS |
| | | | KTSSTTVTLQMTSLTAAD | | RQAPGKGLE | | KPGQPPKLLI | | WYQQKPGQ |
| | | | TATYFCARDYDYTAYAY | | WIACIYTGSS | | YRASTLASGV | | PPKLLIYRA |
| | | | GIMSLWGPGTLVTVSSAST | | GSTSYASWA | | PSRFKGSGSG | | STLASGVPS |
| | | | KGPSVFPLAPSSKSTSGGT | | KGRFTISKTSS | | TQFTLTISDLE | | RFKGSGSGT |
| | | | AALGCLVKDYFPEPVTVS | | TTVTLQMTSL | | CADAATYYC | | QFTLTISDLE |
| | | | WNSGALTSGVHTFPAVLQ | | TAADTATYFC | | QSNYGSYSTN | | CADAATYY |
| | | | SSGLYSLSSVVTVPSSSLG | | ARDYDYTAY | | YGVTFGGGT | | CQSNYGSYS |
| | | | TQTYICNVNHKPSNTKVD | | AYGIMSLWG | | KVEIKRTVAA | | TNYGVTFG |
| | | | KKVEPKSCDKTHTCPPCP | | PGTLVTVSS | | PSVFIFPPSDE | | GGTKVEIK |
| | | | APELLGGPSVFLFPPKPKD | | | | QLKSGTASVV | | |
| | | | TLMISRTPEVTCVVVDVSH | | | | CLLNNFYPRE | | |
| | | | EDPEVKFNWYVDGVEVH | | | | AKVQWKVDN | | |
| | | | NAKTKPREEQYNSTYRVV | | | | ALQSGNSQES | | |
| | | | SVLTVLHQDWLNGKEYK | | | | VTEQDSKDST | | |
| | | | CKVSNKALPAPIEKTISKA | | | | YSLSSTLTLSK | | |
| | | | KGQPREPQVYTLPPSRDEL | | | | ADYEKHKVY | | |
| | | | TKNQVSLWCLVKGFYPSD | | | | ACEVTHQGLS | | |
| | | | IAVEWESNGQPENNYKTT | | | | SPVTKSFNRG | | |
| | | | PPVLDSDGSFFLYSKLTVD | | | | EC | | |
| | | | KSRWQQGNVFSCSVMHE | | | | | | |
| | | | ALHNHYTQKSLSLSPGK | | | | | | |
| EPI862 | RNF43 | 206 | QEQLVESGGGLVQPEGSL | 207 | QEQLVESGGG | 208 | DVVMTQTP | 209 | DVVMTQTP |
| | | | TLTCTASGFSFSSRYYMC | | LVQPEGSLTL | | ASVSEPVGGT | | ASVSEPVGG |
| | | | WVRQAPGKGLEWIGCIYT | | TCTASGFSFSS | | VTIKCQASQSI | | TVTIKCQAS |
| | | | GSGSTYYASWAKGRVTIS | | RYYMCWVRQ | | YSGLAWYQQ | | QSIYSGLAW |
| | | | KTSSTTVTLQMTSLTAAD | | APGKGLEWIG | | KPGQPPKLLI | | YQQKPGQP |
| | | | TATYFCAREAGSFNLWGP | | CIYTGSGSTY | | YSASKLASGV | | PKLLIYSAS |
| | | | GTLVTVSSASTKGPSVFPL | | YASWAKGRV | | PSRFKGSGSG | | KLASGVPSR |
| | | | APSSKSTSGGTAALGCLV | | TISKTSSTTVT | | TEYTLTISDLE | | FKGSGSGTE |
| | | | KDYFPEPVTVSWNSGALT | | LQMTSLTAA | | CADAATYYC | | YTLTISDLE |
| | | | SGVHTFPAVLQSSGLYSLS | | DTATYFCARE | | QNYYYGISNG | | CADAATYY |
| | | | SVVTVPSSSLGTQTYICNV | | AGSFNLWGP | | WTFGGGTKV | | CQNYYYGIS |
| | | | NHKPSNTKVDKKVEPKSC | | GTLVTVSS | | EIKRTVAAPS | | NGWTFGGG |
| | | | DKTHTCPPCPAPELLGGPS | | | | VFIFPPSDEQL | | TKVEIK |
| | | | VFLFPPKPKDTLMISRTPE | | | | KSGTASVVCL | | |
| | | | VTCVVVDVSHEDPEVKFN | | | | LNNFYPREAK | | |
| | | | WYVDGVEVHNAKTKPRE | | | | VQWKVDNAL | | |
| | | | EQYNSTYRVVSVLTVLHQ | | | | QSGNSQESVT | | |
| | | | DWLNGKEYKCKVSNKAL | | | | EQDSKDSTYS | | |
| | | | PAPIEKTISKAKGQPREPQ | | | | LSSTLTLSKA | | |
| | | | VYTLPPSRDELTKNQVSL | | | | DYEKHKVYA | | |
| | | | WCLVKGFYPSDIAVEWES | | | | CEVTHQGLSS | | |
| | | | NGQPENNYKTTPPVLDSD | | | | PVTKSFNRGE | | |
| | | | GSFFLYSKLTVDKSRWQQ | | | | C | | |
| | | | GNVFSCSVMHEALHNHYT | | | | | | |
| | | | QKSLSLSPGK | | | | | | |
| EPI1007 | RNF43 | 274 | QVQLKESGPGLVQPSQTL | 275 | QVQLKESGPG | 276 | DTVLTQSPA | 277 | DTVLTQSPA |
| | | | SLTCTVSGFSLTTYSVHW | | LVQPSQTLSL | | LAVSPGERVT | | LAVSPGERV |
| | | | VRQHSGKNLEWMGRMW | | TCTVSGFSLT | | ISCRASESVSK | | TISCRASES |
| | | | TAGDTSYNSAFTSRLNIFR | | TYSVHWVRQ | | LMHWYQQRP | | VSKLMHWY |
| | | | DTSKSQVFLKMNSLQTED | | HSGKNLEWM | | GQQPQLLIYL | | QQRPGQQP |
| | | | TGTYYCARSSYTSGYPFDS | | GRMWTAGDT | | TSHLASGVPA | | QLLIYLTSH |
| | | | WGQGVMVTVSSASTKGPS | | SYNSAFTSRL | | RFSGSGSGTD | | LASGVPARF |
| | | | VFPLAPSSKSTSGGTAALG | | NIFRDTSKSQ | | FTLTIDPVEA | | SGSGSGTDF |
| | | | CLVKDYFPEPVTVSWNSG | | VFLKMNSLQT | | DDTATYYCQ | | TLTIDPVEA |
| | | | ALTSGVHTFPAVLQSSGLY | | EDTGTYYCA | | QSRNDPTFGA | | DDTATYYC |
| | | | SLSSVVTVPSSSLGTQTYIC | | RSSYTSGYPF | | GTKLELKRTV | | QQSRNDPTF |
| | | | NVNHKPSNTKVDKKVEPK | | DSWGQGVMV | | AAPSVFIFPPS | | GAGTKLEL |
| | | | SCDKTHTCPPCPAPELLGG | | TVSS | | DEQLKSGTAS | | K |
| | | | PSVFLFPPKPKDTLMISRTP | | | | VVCLLNNFYP | | |
| | | | EVTCVVVDVSHEDPEVKF | | | | REAKVQWKV | | |
| | | | NWYVDGVEVHNAKTKPR | | | | DNALQSGNS | | |
| | | | EEQYNSTYRVVSVLTVLH | | | | QESVTEQDSK | | |
| | | | QDWLNGKEYKCKVSNKA | | | | DSTYSLSSTLT | | |
| | | | LPAPIEKTISKAKGQPREPQ | | | | LSKADYEKH | | |
| | | | VYTLPPSRDELTKNQVSL | | | | KVYACEVTH | | |
| | | | WCLVKGFYPSDIAVEWES | | | | QGLSSPVTKS | | |
| | | | NGQPENNYKTTPPVLDSD | | | | FNRGEC | | |
| | | | GSFFLYSKLTVDKSRWQQ | | | | | | |
| | | | GNVFSCSVMHEALHNHYT | | | | | | |
| | | | QKSLSLSPGKGGSHHHHHH | | | | | | |
| EPI1008 | RNF43 | 278 | EVQLVESGGGLVQPGGSL | 279 | EVQLVESGGG | 280 | DIQMTQSPS | 281 | DIQMTQSPS |
| | | | RLSCVVSGFTFSYYDMHW | | LVQPGGSLRL | | SLSASVGDRV | | SLSASVGDR |
| | | | VRQVTGKGLEWVSAIGTA | | SCVVSGFTFS | | TITCRASQSIS | | VTITCRASQ |
| | | | GATYYPGSVKGRFTISREN | | YYDMHWVR | | SYLNWYQQK | | SISSYLNWY |
| | | | AKNSLYLQMNSLRAGDTA | | QVTGKGLEW | | PGKAPKLLIY | | QQKPGKAP |
| | | | VYYCARDRGYSGYDAYY | | VSAIGTAGAT | | AASSLQSGVP | | KLLIYAASS |
| | | | FDFWGQGTLVTVSSASTK | | YYPGSVKGRF | | SRFSGSGSGT | | LQSGVPSRF |
| | | | GPSVFPLAPSSKSTSGGTA | | TISRENAKNS | | DFTLTISSLQP | | SGSGSGTDF |
| | | | ALGCLVKDYFPEPVTVSW | | LYLQMNSLR | | EDFATYYCQ | | TLTISSLQPE |
| | | | NSGALTSGVHTFPAVLQSS | | AGDTAVYYC | | QSYSTPPTFG | | DFATYYCQ |
| | | | GLYSLSSVVTVPSSSLGTQ | | ARDRGYSGY | | QGTKVEIKRT | | QSYSTPPTF |
| | | | TYICNVNHKPSNTKVDKK | | DAYYFDFWG | | VAAPSVFIFPP | | GQGTKVEIK |
| | | | VEPKSCDKTHTCPPCPAPE | | QGTLVTVSS | | SDEQLKSGTA | | |
| | | | LLGGPSVFLFPPKPKDTLM | | | | SVVCLLNNFY | | |
| | | | ISRTPEVTCVVVDVSHEDP | | | | PREAKVQWK | | |
| | | | EVKFNWYVDGVEVHNAK | | | | VDNALQSGN | | |
| | | | TKPREEQYNSTYRVVSVL | | | | SQESVTEQDS | | |
| | | | TVLHQDWLNGKEYKCKV | | | | KDSTYSLSST | | |
| | | | SNKALPAPIEKTISKAKGQ | | | | LTLSKADYEK | | |
| | | | PREPQVYTLPPSRDELTKN | | | | HKVYACEVT | | |
| | | | QVSLWCLVKGFYPSDIAV | | | | HQGLSSPVTK | | |
| | | | EWESNGQPENNYKTTPPV | | | | SFNRGEC | | |
| | | | LDSDGSFFLYSKLTVDKSR | | | | | | |
| | | | WQQGNVFSCSVMHEALH | | | | | | |
| | | | NHYTQKSLSLSPGKGGSH | | | | | | |
| | | | HHHHH | | | | | | |
| EPI1009 | RNF43 | 282 | EVQLVQSGGGLVQPGGSL | 283 | EVQLVQSGG | 284 | DIQMTQSPS | 285 | DIQMTQSPS |
| | | | RLSCAASGFTFSYYDMHW | | GLVQPGGSLR | | SLSASVGDRV | | SLSASVGDR |
| | | | VRQVTGKGLEWVSTIGAT | | LSCAASGFTF | | TITCRASQSIS | | VTITCRASQ |
| | | | GDTYYSDSVKGRFTISRQN | | SYYDMHWVR | | SYLNWYQQK | | SISSYLNWY |
| | | | AKNSLYLQINSLRAGDTA | | QVTGKGLEW | | PGKAPKLLIY | | QQKPGKAP |
| | | | VYYCVRDRGYIGYDSYYF | | VSTIGATGDT | | AASSLQSGVP | | KLLIYAASS |
| | | | DNWGQGTLVTVSSASTKG | | YYSDSVKGRF | | SRFSGSGSGT | | LQSGVPSRF |
| | | | PSVFPLAPSSKSTSGGTAA | | TISRQNAKNS | | DFTLTISSLQP | | SGSGSGTDF |
| | | | LGCLVKDYFPEPVTVSWN | | LYLQINSLRA | | EDFATYYCQ | | TLTISSLQPE |
| | | | SGALTSGVHTFPAVLQSSG | | GDTAVYYCV | | QSYSTPPTFG | | DFATYYCQ |
| | | | LYSLSSVVTVPSSSLGTQT | | RDRGYIGYDS | | QGTKVEIKRT | | QSYSTPPTF |
| | | | YICNVNHKPSNTKVDKKV | | YYFDNWGQG | | VAAPSVFIFPP | | GQGTKVEIK |
| | | | EPKSCDKTHTCPPCPAPEL | | TLVTVSS | | SDEQLKSGTA | | |
| | | | LGGPSVFLFPPKPKDTLMI | | | | SVVCLLNNFY | | |
| | | | SRTPEVTCVVVDVSHEDP | | | | PREAKVQWK | | |
| | | | EVKFNWYVDGVEVHNAK | | | | VDNALQSGN | | |
| | | | TKPREEQYNSTYRVVSVL | | | | SQESVTEQDS | | |
| | | | TVLHQDWLNGKEYKCKV | | | | KDSTYSLSST | | |
| | | | SNKALPAPIEKTISKAKGQ | | | | LTLSKADYEK | | |
| | | | PREPQVYTLPPSRDELTKN | | | | HKVYACEVT | | |
| | | | QVSLWCLVKGFYPSDIAV | | | | HQGLSSPVTK | | |
| | | | EWESNGQPENNYKTTPPV | | | | SFNRGEC | | |
| | | | LDSDGSFFLYSKLTVDKSR | | | | | | |
| | | | WQQGNVFSCSVMHEALH | | | | | | |
| | | | NHYTQKSLSLSPGKGGSH | | | | | | |
| | | | HHHHH | | | | | | |
| EPI864 | RNF128 | 212 | QVQLQESGGGLVQAGGSL | 213 | QVQLQESGG | | | | |
| | | | RLSCAASGNISVQLDMGW | | GLVQAGGSL | | | | |
| | | | YRQAPGKEREFVAAINQG | | RLSCAASGNI | | | | |
| | | | TTTYYADSVKGRFTISRDN | | SVQLDMGWY | | | | |
| | | | AKNTVYLQMNSLKPEDTA | | RQAPGKEREF | | | | |
| | | | VYYCAVYLYDIWNHPYW | | VAAINQGTTT | | | | |
| | | | GQGTQVTVSSGGGGSDKT | | YYADSVKGR | | | | |
| | | | HTCPPCPAPELLGGPSVFL | | FTISRDNAKN | | | | |
| | | | FPPKPKDTLMISRTPEVTC | | TVYLQMNSL | | | | |
| | | | VVVDVSHEDPEVKFNWY | | KPEDTAVYY | | | | |
| | | | VDGVEVHNAKTKPREEQY | | CAVYLYDIW | | | | |
| | | | NSTYRVVSVLTVLHQDWL | | NHPYWGQGT | | | | |
| | | | NGKEYKCKVSNKALPAPI | | QVTVSS | | | | |
| | | | EKTISKAKGQPREPQVYTL | | | | | | |
| | | | PPSRDELTKNQVSLWCLV | | | | | | |
| | | | KGFYPSDIAVEWESNGQP | | | | | | |
| | | | ENNYKTTPPVLDSDGSFFL | | | | | | |
| | | | YSKLTVDKSRWQQGNVFS | | | | | | |
| | | | CSVMHEALHNHYTQKSLS | | | | | | |
| | | | LSPGKGGSHHHHHH | | | | | | |
| EPI865 | RNF128 | 214 | QVQLQESGGGLVQAGGSL | 215 | QVQLQESGG | | | | |
| | | | RLSCAASGSISGGKGMGW | | GLVQAGGSL | | | | |
| | | | YRQAPGKEREFVAAIGSG | | RLSCAASGSIS | | | | |
| | | | AITYYADSVKGRFTISRDN | | GGKGMGWY | | | | |
| | | | AKNTVYLQMNSLKPEDTA | | RQAPGKEREF | | | | |
| | | | VYYCAVYTTALDEYPYW | | VAAIGSGAIT | | | | |
| | | | GQGTQVTVSSGGGGSDKT | | YYADSVKGR | | | | |
| | | | HTCPPCPAPELLGGPSVFL | | FTISRDNAKN | | | | |
| | | | FPPKPKDTLMISRTPEVTC | | TVYLQMNSL | | | | |
| | | | VVVDVSHEDPEVKFNWY | | KPEDTAVYY | | | | |
| | | | VDGVEVHNAKTKPREEQY | | CAVYTTALDE | | | | |
| | | | NSTYRVVSVLTVLHQDWL | | YPYWGQGTQ | | | | |
| | | | NGKEYKCKVSNKALPAPI | | VTVSS | | | | |
| | | | EKTISKAKGQPREPQVYTL | | | | | | |
| | | | PPSRDELTKNQVSLWCLV | | | | | | |
| | | | KGFYPSDIAVEWESNGQP | | | | | | |
| | | | ENNYKTTPPVLDSDGSFFL | | | | | | |
| | | | YSKLTVDKSRWQQGNVFS | | | | | | |
| | | | CSVMHEALHNHYTQKSLS | | | | | | |
| | | | LSPGKGGSHHHHHH | | | | | | |
| EPI1013 | RNF130 | 298 | QVQLQESGGGLVQAGGSL | 299 | QVQLQESGG | | | | |
| | | | RLSCAASGYISGYYVMGW | | GLVQAGGSL | | | | |
| | | | YRQAPGKEREFVASISYGA | | RLSCAASGYI | | | | |
| | | | STYYADSVKGRFTISRDNA | | SGYYVMGWY | | | | |
| | | | KNTVYLQMNSLKPEDTAV | | RQAPGKEREF | | | | |
| | | | YYCAVDFDSNYAHTYWG | | VASISYGAST | | | | |
| | | | QGTQVTVSSGGGGSDKTH | | YYADSVKGR | | | | |
| | | | TCPPCPAPELLGGPSVFLFP | | FTISRDNAKN | | | | |
| | | | PKPKDTLMISRTPEVTCVV | | TVYLQMNSL | | | | |
| | | | VDVSHEDPEVKFNWYVD | | KPEDTAVYY | | | | |
| | | | GVEVHNAKTKPREEQYNS | | CAVDFDSNY | | | | |
| | | | TYRVVSVLTVLHQDWLN | | AHTYWGQGT | | | | |
| | | | GKEYKCKVSNKALPAPIE | | QVTVSS | | | | |
| | | | KTISKAKGQPREPQVYTLP | | | | | | |
| | | | PSRDELTKNQVSLWCLVK | | | | | | |
| | | | GFYPSDIAVEWESNGQPE | | | | | | |
| | | | NNYKTTPPVLDSDGSFFLY | | | | | | |
| | | | SKLTVDKSRWQQGNVFSC | | | | | | |
| | | | SVMHEALHNHYTQKSLSL | | | | | | |
| | | | SPGKGGSHHHHHH | | | | | | |
| EPI1014 | RNF130 | 300 | QVQLQESGGGLVQAGGSL | 301 | QVQLQESGG | | | | |
| | | | RLSCAASGTISFIGYMGWY | | GLVQAGGSL | | | | |
| | | | RQAPGKERELVASIASGTS | | RLSCAASGTIS | | | | |
| | | | TYYADSVKGRFTISRDNA | | FIGYMGWYR | | | | |
| | | | KNTVYLQMNSLKPEDTAV | | QAPGKERELV | | | | |
| | | | YYCAATQYIQDVHRYWG | | ASIASGTSTY | | | | |
| | | | QGTQVTVSSGGGGSDKTH | | YADSVKGRFT | | | | |
| | | | TCPPCPAPELLGGPSVFLFP | | ISRDNAKNTV | | | | |
| | | | PKPKDTLMISRTPEVTCVV | | YLQMNSLKPE | | | | |
| | | | VDVSHEDPEVKFNWYVD | | DTAVYYCAA | | | | |
| | | | GVEVHNAKTKPREEQYNS | | TQYIQDVHRY | | | | |
| | | | TYRVVSVLTVLHQDWLN | | WGQGTQVTV | | | | |
| | | | GKEYKCKVSNKALPAPIE | | SS | | | | |
| | | | KTISKAKGQPREPQVYTLP | | | | | | |
| | | | PSRDELTKNQVSLWCLVK | | | | | | |
| | | | GFYPSDIAVEWESNGQPE | | | | | | |
| | | | NNYKTTPPVLDSDGSFFLY | | | | | | |
| | | | SKLTVDKSRWQQGNVFSC | | | | | | |
| | | | SVMHEALHNHYTQKSLSL | | | | | | |
| | | | SPGKGGSHHHHHH | | | | | | |

The bispecifics used in this example comprise a second binding arm comprising the sequences listed in Table 8. The sequences listed in Table 8 was paired with each antibody listed in Table 7.

TABLE 8
Binding arm 2 targets and sequences

| | | SEQ | | SEQ | | SEQ | | SEQ | |
|---|---|---|---|---|---|---|---|---|---|
| | Arm 1 | ID | | ID | | ID | LC | ID | VL |
| ID | Target | NO | HC sequence | NO | VH sequence | NO | sequence | NO | sequence |
| AA235 | EGFR | 724 | QVKLVESGGGVVRPGGSL | 725 | QVKLVESGG | | | | |
| | | | TLSCAASGRTSRSYGMGW | | GVVRPGGSLT | | | | |
| | | | FRQAPGKEREFVSGISWRG | | LSCAASGRTS | | | | |
| | | | DSTGYADSVKGRFTISRDN | | RSYGMGWFR | | | | |
| | | | AKNSLYLQMNSLRAEDTA | | QAPGKEREFV | | | | |
| | | | LYYCAAAAGSAWYGTLY | | SGISWRGDST | | | | |
| | | | EYDYWGQGTLVTVSSGG | | GYADSVKGR | | | | |
| | | | GGSDKTHTCPPCPAPELLG | | FTISRDNAKN | | | | |
| | | | GPSVFLFPPKPKDTLMISR | | SLYLQMNSLR | | | | |
| | | | TPEVTCVVVDVSHEDPEV | | AEDTALYYC | | | | |
| | | | KFNWYVDGVEVHNAKTK | | AAAAGSAWY | | | | |
| | | | PREEQYNSTYRVVSVLTV | | GTLYEYDYW | | | | |
| | | | LHQDWLNGKEYKCKVSN | | GQGTLVTVSS | | | | |
| | | | KALPAPIEKTISKAKGQPR | | | | | | |
| | | | EPQVYTLPPSRDELTKNQV | | | | | | |
| | | | SLSCAVKGFYPSDIAVEWE | | | | | | |

SNGQPENNYKTTPPVLDS
DGSFFLVSKLTVDKSRWQ
QGNVFSCSVMHEALHNH
YTQKSLSLSPGK

The screen was performed on both NCIH1975 (non-small cell lung cancer) and HT29 (colorectal cancer) cell lines using the methods described in Example 8. Briefly, cells were seeded in 96-well plates and incubated overnight at 37° C. and 5% CO₂. The next morning, cells were treated with 500 nM of the bispecific or control antibody. After 24 hours of treatment, cells were harvested using a dissociation reagent, stained using a fluorescently labeled anti-EGFR antibody, and acquired on a Cytek Northern Lights flow cytometer. Percent EGFR cell surface removal was calculated using an untreated control sample after accounting for background with an isotype control.

Results of screen identified degrader protein groups and specific molecular epitopes that, when paired with EGFR in a bispecific antibody format, demonstrated improved capacity to induce EGFR cell surface removal as compared to Palivizumab x Mav2, a bispecific pairing EGFR with a non-targeting control arm (FIGS. 3A and 3B). A number of efficacious bispecific antibodies arose that were unique to the cancer type of the cell line used. In NCIH1975 cells (non-small cell lung cancer), MUC1, ITGB6, and TROP2 were efficacious in removing EGFR from the cell surface (FIG. 3A). For HT29 (colorectal cancer), CD276, RNF43, and MST1R were efficacious in removing EGFR from the cell surface (FIG. 3B). Additionally, EpCAM, CD71, LGR5, and HER3 were efficacious in removing EGFR from the cell surface across cell lines. These experiments demonstrate the cell-specificity of EGFR degradation using various bispecific antibody pairs.

### Example 9—EGFR Cell Surface Removal of Non-Mav2 EGFR-Targeting Bispecific Antibodies

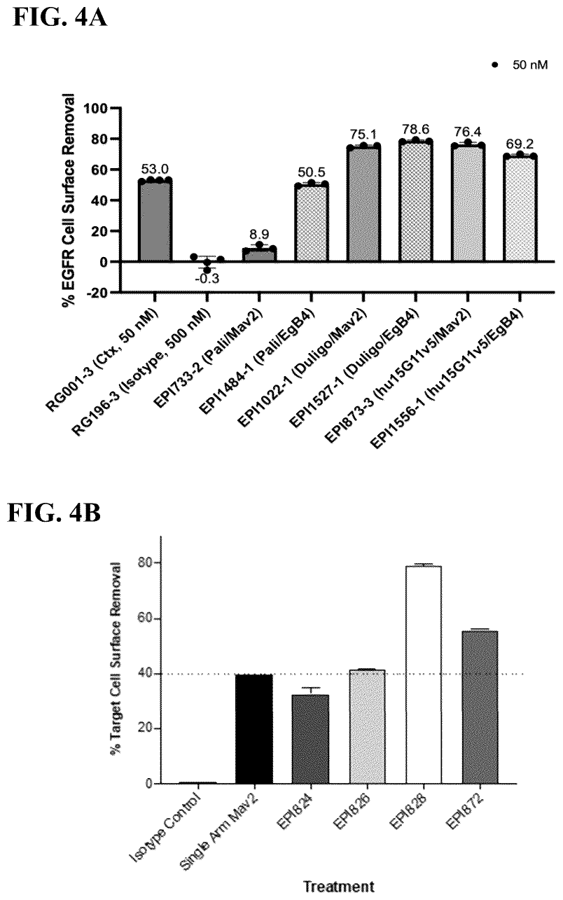

While the majority of molecules included in the screen employed Mav2 as the EGFR binding arm in the bispecific antibody, a binder that employed EgB4 as an EGFR binding arm was also tested in a cell surface removal assay (FIG. 4). EgB4 is an EGFR binding arm with the same binding affinity to EGFR as Mav2 (9.8 nM), but does not have EGF-blocking properties, indicating a difference in epitope compared to Mav2. The Mav2 and EgB4 binders were delivered as VHH (sdAb) format. Duligotuzumab (HER3 x EGFR) or hu15G11v5 (CD71) binding domains were used for the degrader protein binding arm. Additionally, RG001-3 (Cetuximab), RG196-3 (an IgGI isotype control), EPI733-2 (Mav2 x RSV), and EPI484-1 (EgB4 x RSV) were tested for comparison (Table 9; Table 10)

TABLE 9
EGFR molecule information

| Molecule ID | Name | Arm 1 Binder | Arm 2 Binder |
|---|---|---|---|
| RG001-3 | Cetuximab (MCE) | EGFR | (IgG) |
| RG196-3 | Human IgG1 Isotype | | |
| | Control (BioXcell) | | |
| EPI733-1 | Palivizumab/Mav2 | EGFR (Mav2) | RSV F |
| EPI1484-1 | Palivizumab/EgB4 | EGFR (EgB4) | RSV F |
| EPI1022-1 | Duligotuzumab/Mav2 | EGFR (Mav2) | HER3/EGFR |
| EPI1527-1 | Duligotuzumab/EgB4 | EGFR (EgB4) | HER3/EGFR |
| EPI873-2 | hu15G11v5/Mav2 | EGFR (Mav2) | CD71 |
| EPI1556-1 | hu15G11v5/EgB4 | EGFR (EgB4) | CD71 |

TABLE 10
Exemplary EgB4 bispecific sequence information

| | | SEQ | | SEQ | | SEQ | | SEQ | | | SEQ | | SEQ | |
|---|---|---|---|---|---|---|---|---|---|---|---|---|---|---|
| | Arm 1 | ID | | ID | VH | ID | LC | ID | VL | Arm 2 | ID | HC | ID | VH |
| ID | Target | NO | HC sequence | NO | sequence | NO | sequence | NO | sequence | Target | NO | sequence | NO | sequence |
| EPI1484 | RSV F | 660 | QVTLRESGPAL | 661 | QVTLR | 662 | DIQMTQ | 663 | DIQMT | EGFR | 664 | QVQLQESGG | 665 | QVQLQ |
| | Protein | | VKPTQTLTLTCT | | ESGPAL | | SPSTLSA | | QSPSTL | | | GSVQAGGSL | | ESGGG |
| | | | FSGFSLSTSGMS | | VKPTQ | | SVGDRV | | SASVG | | | KLSCAASGRS | | SVQAG |
| | | | VGWIRQPPGKA | | TLTLTC | | TITCKCQ | | DRVTIT | | | FSTYAMGWF | | GSLKL |
| | | | LEWLADIWWD | | TFSGFS | | LSVGYM | | CKCQL | | | RQAPGODREF | | SCAAS |
| | | | DKKDYNPSLKS | | LSTSG | | HWYQQ | | SVGYM | | | VATISWTDST | | GRSFST |
| | | | RLTISKDTSKNQ | | MSVGW | | KPGKAP | | HWYQ | | | DYADSVKGR | | YAMG |
| | | | VVLKVTNMDPA | | IRQPPG | | KLLIYDT | | QKPGK | | | FTISRDNAKN | | WFRQA |
| | | | DTATYYCARSM | | KALEW | | SKLASG | | APKLLI | | | TGYLQMNSL | | PGQDR |
| | | | ITNWYFDVWGA | | LADIW | | VPSRFSG | | YDTSK | | | KPEDTAVYY | | EFVATI |
| | | | GTTVTVSSASTK | | WDDKK | | SGSGTEF | | LASGV | | | CAADRWASS | | SWTDS |
| | | | GPSVFPLAPSSK | | DYNPSL | | TLTISSL | | PSRFSG | | | RRNVDYDYW | | TDYAD |
| | | | STSGGTAALGCL | | KSRLTI | | QPDDFA | | SGSGT | | | GQGTQVTVSS | | SVKGR |
| | | | VKDYFPEPVTVS | | SKDTSK | | TYYCFQ | | EFTLTI | | | GGGGSDKTH | | FTISRD |
| | | | WNSGALTSGVH | | NQVVL | | GSGYPFT | | SSLQPD | | | TCPPCPAPELL | | NAKNT |
| | | | TFPAVLQSSGLY | | KVTNM | | FGGGTK | | DFATY | | | GGPSVFLFPP | | GYLQM |
| | | | SLSSVVTVPSSS | | DPADT | | LEIKRTV | | YCFQG | | | KPKDTLMISR | | NSLKP |
| | | | LGTQTYICNVN | | ATYYC | | AAPSVFI | | SGYPFT | | | TPEVTCVVVD | | EDTAV |
| | | | HKPSNTKVDKK | | ARSMIT | | FPPSDEQ | | FGGGT | | | VSHEDPEVKF | | YYCAA |
| | | | VEPKSCDKTHT | | NWYFD | | LKSGTAS | | KLEIK | | | NWYVDGVEV | | DRWAS |
| | | | CPPCPAPELLGG | | VWGAG | | VVCLLN | | | | | HNAKTKPREE | | SRRNV |
| | | | PSVFLFPPKPKD | | TTVTVS | | NFYPRE | | | | | QYNSTYRVVS | | DYDY |
| | | | TLMISRTPEVTC | | S | | AKVQW | | | | | VLTVLHQDW | | WGQGT |
| | | | VVVDVSHEDPE | | | | KVDNAL | | | | | LNGKEYKCK | | QVTVS |
| | | | VKFNWYVDGV | | | | QSGNSQ | | | | | VSNKALPAPI | | S |
| | | | EVHNAKTKPRE | | | | ESVTEQ | | | | | EKTISKAKGQ | | |
| | | | EQYNSTYRVVS | | | | DSKDST | | | | | PREPQVYTLP | | |
| | | | VLTVLHQDWLN | | | | YSLSSTL | | | | | PSRDELTKNQ | | |
| | | | GKEYKCKVSNK | | | | TLSKAD | | | | | VSLSCAVKGF | | |
| | | | ALPAPIEKTISKA | | | | YEKHKV | | | | | YPSDIAVEWE | | |
| | | | KGQPREPQVYT | | | | YACEVT | | | | | SNGQPENNY | | |
| | | | LPPSRDELTKNQ | | | | HQGLSSP | | | | | KTTPPVLDSD | | |
| | | | VSLWCLVKGFY | | | | VTKSFN | | | | | GSFFLVSKLT | | |
| | | | PSDIAVEWESNG | | | | RGEC | | | | | VDKSRWQQG | | |
| | | | QPENNYKTTPPV | | | | | | | | | NVFSCSVMHE | | |
| | | | LDSDGSFFLYSK | | | | | | | | | ALHNHYTQK | | |
| | | | LTVDKSRWQQG | | | | | | | | | SLSLSPGK | | |
| | | | NVFSCSVMHEA | | | | | | | | | | | |
| | | | LHNHYTQKSLS | | | | | | | | | | | |
| | | | LSPGKGGSHHH | | | | | | | | | | | |
| | | | HHH | | | | | | | | | | | |
| EPI1488 | CD71 | 666 | QVQLVQSGAEV | 667 | QVQLV | 668 | DIQMTQ | 669 | DIQMT | EGFR | 670 | QVQLQESGG | 671 | QVQLQ |
| | | | KKPGASVKMSC | | QSGAE | | SPSSLSA | | QSPSSL | | | GSVQAGGSL | | ESGGG |
| | | | KASGYTFTSYW | | VKKPG | | SVGDRV | | SASVG | | | KLSCAASGRS | | SVQAG |
| | | | MHWVRQAPGQ | | ASVKM | | TITCSAS | | DRVTIT | | | FSTYAMGWF | | GSLKL |
| | | | GLEWIGAIYPGN | | SCKAS | | SSVYYM | | CSASSS | | | RQAPGQDREF | | SCAAS |
| | | | SETGYAQKFQG | | GYTFTS | | YWFQQK | | VYYM | | | VATISWTDST | | GRSFST |
| | | | RATLTADTSTST | | YWMH | | PGKAPK | | YWFQQ | | | DYADSVKGR | | YAMG |
| | | | AYMELSSLRSED | | WVRQA | | LWIYSTS | | KPGKA | | | FTISRDNAKN | | WFRQA |
| | | | TAVYYCTRENW | | PGQGL | | NLASGV | | PKLWI | | | TGYLQMNSL | | PGQDR |
| | | | DPGFAFWGQGT | | EWIGAI | | PSRFSGS | | YSTSN | | | KPEDTAVYY | | EFVATI |
| | | | LITVSSASTKGPS | | YPGNSE | | GSGTDY | | LASGV | | | CAADRWASS | | SWTDS |
| | | | VFPLAPSSKSTS | | TGYAQ | | TLTISSM | | PSRFSG | | | RRNVDYDYW | | TDYAD |
| | | | GGTAALGCLVK | | KFQGR | | QPEDFAT | | SGSGT | | | GQGTQVTVSS | | SVKGR |
| | | | DYFPEPVTVSW | | ATLTA | | YYCQQR | | DYTLTI | | | GGGGSDKTH | | FTISRD |
| | | | NSGALTSGVHTF | | DTSTST | | RNYPYT | | SSMQP | | | TCPPCPAPELL | | NAKNT |
| | | | PAVLQSSGLYSL | | AYMEL | | FGQGTK | | EDFAT | | | GGPSVFLFPP | | GYLQM |
| | | | SSVVTVPSSSLG | | SSLRSE | | LEIKRTV | | YYCQQ | | | KPKDTLMISR | | NSLKP |
| | | | TQTYICNVNHKP | | DTAVY | | AAPSVFI | | RRNYP | | | TPEVTCVVVD | | EDTAV |
| | | | SNTKVDKKVEP | | YCTRE | | FPPSDEQ | | YTFGQ | | | VSHEDPEVKF | | YYCAA |
| | | | KSCDKTHTCPPC | | NWDPG | | LKSGTAS | | GTKLEI | | | NWYVDGVEV | | DRWAS |
| | | | PAPELLGGPSVF | | FAFWG | | VVCLLN | | K | | | HNAKTKPREE | | SRRNV |
| | | | LFPPKPKDTLMI | | QGTLIT | | NFYPRE | | | | | QYNSTYRVVS | | DYDY |
| | | | SRTPEVTCVVV | | VSS | | AKVQW | | | | | VLTVLHQDW | | WGQGT |
| | | | DVSHEDPEVKF | | | | KVDNAL | | | | | LNGKEYKCK | | QVTVS |
| | | | NWYVDGVEVH | | | | QSGNSQ | | | | | VSNKALPAPI | | s |
| | | | NAKTKPREEQY | | | | ESVTEQ | | | | | EKTISKAKGQ | | |
| | | | NSTYRVVSVLT | | | | DSKDST | | | | | PREPQVYTLP | | |
| | | | VLHQDWLNGKE | | | | YSLSSTL | | | | | PSRDELTKNQ | | |
| | | | YKCKVSNKALP | | | | TLSKAD | | | | | VSLSCAVKGF | | |
| | | | APIEKTISKAKG | | | | YEKHKV | | | | | YPSDIAVEWE | | |
| | | | QPREPQVYTLPP | | | | YACEVT | | | | | SNGQPENNY | | |
| | | | SRDELTKNQVSL | | | | HQGLSSP | | | | | KTTPPVLDSD | | |
| | | | WCLVKGFYPSDI | | | | VTKSFN | | | | | GSFFLVSKLT | | |
| | | | AVEWESNGQPE | | | | RGEC | | | | | VDKSRWQQG | | |
| | | | NNYKTTPPVLDS | | | | | | | | | NVFSCSVMHE | | |
| | | | DGSFFLYSKLTV | | | | | | | | | ALHNHYTQK | | |
| | | | DKSRWQQGNVF | | | | | | | | | SLSLSPGK | | |
| | | | SCSVMHEALHN | | | | | | | | | | | |
| | | | HYTQKSLSLSPG | | | | | | | | | | | |
| | | | K | | | | | | | | | | | |
| EPI1527 | HER3 | 672 | EVQLVESGGGL | 673 | EVQLV | 674 | DIQMT | 675 | DIQMT | EGFR | 676 | QVQLQESGG | 677 | QVQ |
| | | | VQPGGSLRLSCA | | ESGGG | | QSPSSLS | | QSPSSL | | | GSVQAGGSL | | LQES |
| | | | ASGFTLSGDWIH | | LVQPG | | ASVGDR | | SASVG | | | KLSCAASGRS | | GGGS |
| | | | WVRQAPGKGLE | | GSLRLS | | VTITCRA | | DRVTIT | | | FSTYAMGWF | | VQAG |
| | | | WVGEISAAGGY | | CAASG | | SQNIATD | | CRASQ | | | RQAPGODREF | | GSLK |
| | | | TDYADSVKGRF | | FTLSGD | | VAWYQ | | NIATD | | | VATISWTDST | | LSCA |
| | | | TISADTSKNTAY | | WIHWV | | QKPGKA | | VAWY | | | DYADSVKGR | | ASGR |
| | | | LQMNSLRAEDT | | RQAPG | | PKLLIYS | | QQKPG | | | FTISRDNAKN | | SFSTY |
| | | | AVYYCARESRV | | KGLEW | | ASFLYSG | | KAPKL | | | TGYLQMNSL | | AMG |
| | | | SFEAAMDYWG | | VGEISA | | VPSRFSG | | LIYSAS | | | KPEDTAVYY | | WFRQ |
| | | | QGTLVTVSSAST | | AGGYT | | SGSGTDF | | FLYSG | | | CAADRWASS | | APGQ |
| | | | KGPSVFPLAPSS | | DYADS | | TLTISSL | | VPSRFS | | | RRNVDYDYW | | DREF |
| | | | KSTSGGTAALG | | VKGRF | | QPEDFAT | | GSGSG | | | GQGTQVTVSS | | VATIS |
| | | | CLVKDYFPEPVT | | TISADT | | YYCQQS | | TDFTLT | | | GGGGSDKTH | | WTDS |
| | | | VSWNSGALTSG | | SKNTA | | EPEPYTF | | ISSLQP | | | TCPPCPAPELL | | TDYA |
| | | | VHTFPAVLQSSG | | YLQMN | | GQGTKV | | EDFAT | | | GGPSVFLFPP | | DSVK |
| | | | LYSLSSVVTVPS | | SLRAED | | EIKRTVA | | YYCQQ | | | KPKDTLMISR | | GRFTI |
| | | | SSLGTQTYICNV | | TAVYY | | APSVFIF | | SEPEPY | | | TPEVTCVVVD | | ISRDN |
| | | | NHKPSNTKVDK | | CARESR | | PPSDEQL | | TFGQG | | | VSHEDPEVKF | | AKNT |
| | | | KVEPKSCDKTH | | VSFEAA | | KSGTAS | | TKVEIK | | | NWYVDGVEV | | GYLQ |
| | | | TCPPCPAPELLG | | MDYW | | VVCLLN | | | | | HNAKTKPREE | | MNSL |
| | | | GPSVFLFPPKPK | | GQGTL | | NFYPRE | | | | | QYNSTYRVVS | | KPED |
| | | | DTLMISRTPEVT | | VTVSS | | AKVQW | | | | | VLTVLHQDW | | TAVY |
| | | | CVVVDVSHEDP | | | | KVDNAL | | | | | LNGKEYKCK | | YCAA |
| | | | EVKFNWYVDG | | | | QSGNSQ | | | | | VSNKALPAPI | | DRWA |
| | | | VEVHNAKTKPR | | | | ESVTEQ | | | | | EKTISKAKGQ | | SSRRN |
| | | | EEQYNSTYRVV | | | | DSKDST | | | | | PREPQVYTLP | | VDYD |
| | | | SVLTVLHQDWL | | | | YSLSSTL | | | | | PSRDELTKNQ | | YWGQ |
| | | | NGKEYKCKVSN | | | | TLSKAD | | | | | VSLSCAVKGF | | GTQV |
| | | | KALPAPIEKTIS | | | | YEKHKV | | | | | YPSDIAVEWE | | TVSS |
| | | | KAKGQPREPQVY | | | | YACEVT | | | | | SNGQPENNY | | |
| | | | TLPPSRDELTKN | | | | HQGLSSP | | | | | KTTPPVLDSD | | |
| | | | QVSLWCLVKGF | | | | VTKSFN | | | | | GSFFLVSKLT | | |
| | | | YPSDIAVEWESN | | | | RGEC | | | | | VDKSRWQQG | | |
| | | | GQPENNYKTTPP | | | | | | | | | NVFSCSVMHE | | |
| | | | VLDSDGSFFLYS | | | | | | | | | ALHNHYTQK | | |
| | | | KLTVDKSRWQQ | | | | | | | | | SLSLSPGK | | |
| | | | GNVFSCSVMHE | | | | | | | | | | | |
| | | | ALHNHYTQKSL | | | | | | | | | | | |
| | | | SLSPGK | | | | | | | | | | | |
| EPI1556 | CD71 | 678 | EVQLVQSGAEV | 679 | EVQLV | 680 | DIQMT | 681 | DIQMT | EGFR | 682 | QVQLQESGG | 683 | QVQ |
| | | | KKPGASVKVSC | | QSGAE | | QSPSSLS | | QSPSSL | | | GSVQAGGSL | | LQES |
| | | | KASGYTFTSYW | | VKKPG | | ASVGDR | | SASVG | | | KLSCAASGRS | | GGGS |
| | | | MHWVRQAPGQ | | ASVKV | | VTITCRA | | DRVTIT | | | FSTYAMGWF | | VQAG |
| | | | RLEWIGEINPTN | | SCKAS | | SDNLYS | | CRASD | | | RQAPGQDREF | | GSLK |
| | | | GRTNYIEKFKSR | | GYTFTS | | NLAWYQ | | NLYSN | | | VATISWTDST | | LSCA |
| | | | ATLTVDKSAST | | YWMH | | QKPGKS | | LAWYQ | | | DYADSVKGR | | ASGR |
| | | | AYMELSSLRSED | | WVRQA | | PKLLVY | | QKPGK | | | FTISRDNAKN | | SFSTY |
| | | | TAVYYCARGTR | | PGQRLE | | DATNLA | | SPKLL | | | TGYLQMNSL | | AMG |
| | | | AYHYWGQGTM | | WIGEIN | | DGVPSRF | | VYDAT | | | KPEDTAVYY | | WFRQ |
| | | | VTVSSASTKGPS | | PTNGRT | | SGSGSGT | | NLADG | | | CAADRWASS | | APGQ |
| | | | VFPLAPSSKSTS | | NYIEKF | | DYTLTIS | | VPSRFS | | | RRNVDYDYW | | DREF |
| | | | GGTAALGCLVK | | KSRATL | | SLOPEDF | | GSGSG | | | GQGTQVTVSS | | VATIS |
| | | | DYFPEPVTVSW | | TVDKS | | ATYYCQ | | TDYTL | | | GGGGSDKTH | | WTDS |
| | | | NSGALTSGVHTF | | ASTAY | | HFWGTP | | TISSLQ | | | TCPPCPAPELL | | TDYA |
| | | | PAVLQSSGLYSL | | MELSSL | | LTFGQG | | PEDFA | | | GGPSVFLFPP | | DSVK |
| | | | SSVVTVPSSSLG | | RSEDTA | | TKVEIKR | | TYYCQ | | | KPKDTLMISR | | GRFTI |
| | | | TQTYICNVNHKP | | VYYCA | | TVAAPS | | HFWGT | | | TPEVTCVVVD | | SRDN |
| | | | SNTKVDKKVEP | | RGTRA | | VFIFPPS | | PLTFG | | | VSHEDPEVKF | | AKNT |
| | | | KSCDKTHTCPPC | | YHYWG | | DEQLKS | | QGTKV | | | NWYVDGVEV | | GYLQ |
| | | | PAPELLGGPSVF | | QGTMV | | GTASVV | | EIK | | | HNAKTKPREE | | MNSL |
| | | | LFPPKPKDTLMI | | TVSS | | CLLNNF | | | | | QYNSTYRVVS | | KPED |
| | | | SRTPEVTCVVV | | | | YPREAK | | | | | VLTVLHQDW | | TAVY |
| | | | DVSHEDPEVKF | | | | VQWKV | | | | | LNGKEYKCK | | YCAA |
| | | | NWYVDGVEVH | | | | DNALOS | | | | | VSNKALPAPI | | DRWA |
| | | | NAKTKPREEQY | | | | GNSQES | | | | | EKTISKAKGQ | | SSRRN |
| | | | NSTYRVVSVLT | | | | VTEQDS | | | | | PREPQVYTLP | | VDYD |
| | | | VLHQDWLNGKE | | | | KDSTYSL | | | | | PSRDELTKNQ | | YWGQ |
| | | | YKCKVSNKALP | | | | SSTLTLS | | | | | VSLSCAVKGF | | GTQV |
| | | | APIEKTISKAKG | | | | KADYEK | | | | | YPSDIAVEWE | | TVSS |
| | | | QPREPQVYTLPP | | | | HKVYAC | | | | | SNGQPENNY | | |
| | | | SRDELTKNQVSL | | | | EVTHQG | | | | | KTTPPVLDSD | | |
| | | | WCLVKGFYPSDI | | | | LSSPVTK | | | | | GSFFLVSKLT | | |
| | | | AVEWESNGQPE | | | | SFNRGEC | | | | | VDKSRWQQG | | |
| | | | NNYKTTPPVLDS | | | | | | | | | NVFSCSVMHE | | |
| | | | DGSFFLYSKLTV | | | | | | | | | ALHNHYTQK | | |
| | | | DKSRWQQGNVF | | | | | | | | | SLSLSPGK | | |
| | | | SCSVMHEALHN | | | | | | | | | | | |
| | | | HYTQKSLSLSPG | | | | | | | | | | | |
| | | | K | | | | | | | | | | | |

The sequences listed in Table 10 (SEQ ID NOs: 660-683) are amino acid molecules. The sequences listed in Table 10 (SEQ ID NOs: 660-683) are amino acid molecules that are synthetic constructs. The sequences listed in Table 10 (SEQ ID NOs: 660-683) for HC sequences (heavy chain), VH sequence (variable heavy chain sequence), LC sequences (light chain), VL sequence (variable light chain sequence) are amino acid molecules that are synthetic constructs.

The screen was performed in NCIH1975 (non-small cell lung cancer) cells or HT29 (colorectal cancer) cells using the methods described in Example 8. Briefly, cells were seeded in 96-well plates and incubated overnight at 37° C. and 5% CO₂. The next morning, cells were treated with 50 nM of therapeutic bispecific or control antibody. After 24 hours of treatment, cells were harvested using a dissociation reagent, stained using a fluorescently labeled anti-EGFR antibody, and acquired on a Cytek Northern Lights flow cytometer. Percent EGFR cell surface removal was calculated using an untreated control sample after accounting for background with an isotype control.

The bispecifics with EgB4 binding domains showed similar levels of EGFR cell surface removal as bispecifics with Mav2 binding domains (FIGS. 4A-4B). For duligotuzumab (HER3), EGFR cell surface removal of the bispecific with Mav2 binding domain (EPI1022-1) and the bispecific with EgB4 binding domain (EPI1527-1) were 75.1% and 78.6%, respectively. For hu15G11v5 (CD71), EGFR cell surface removal of the bispecific with Mav2 binding domain (EPI873-3) and the bispecific with EgB4 binding domain (EPI1556-1) were 76.4% and 69.2%, respectively. This demonstrates the removal of cell surface EGFR can occur using different EGFR binder arms. Furthermore, an EGFR binder paired to a degrading receptor binder in a bispecific antibody format can induce robust cell surface removal of EGFR.

### Example 10—Bispecifics Show Synergistic Internalization Activity with Both an EGFR-Binding Domain and a Degrader Protein Binding Domain

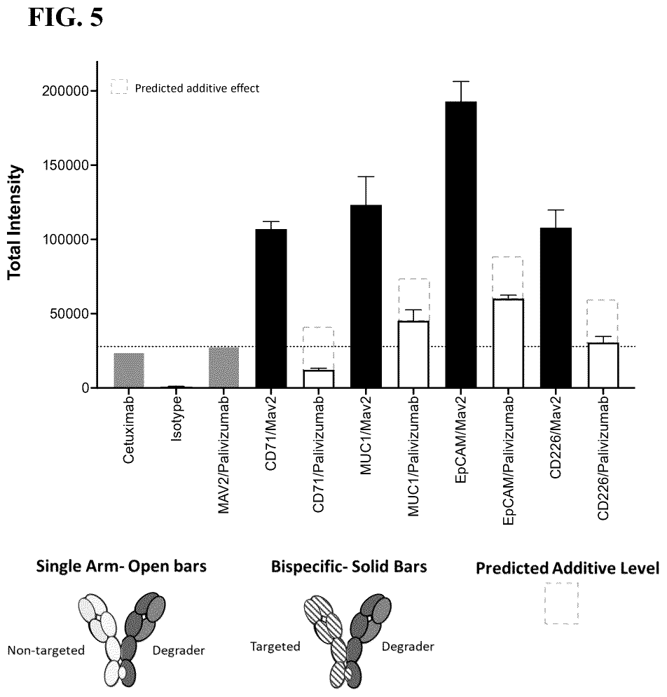

To demonstrate that bispecifics have synergistic internalization activity with both an EGFR-binding domain and a degrader protein binding domain, the internalization activity of bispecific antibodies with both an EGFR-binding domain and a degrader binding domain was compared to the internalization activity of antibodies with an RSV-binding domain (palivizumab) and a degrader binding domain (FIG. 5). Antibodies that bind to different target proteins, including CD71 (bispecific antibody: EPI1015; single arm antibody: EPI1177), MUC1 (EPI828; EPI1123), EpCAM (EPI847; EPI1149), and CD226 (EPI835; EPI1137), were tested. Additionally, Cetuximab (RG001-3), an IgGI isotype control (RG196-1), and Mav2/Palivizumab (EGFR x RSV; EPI733-1) were tested as an EGFR only control, a negative control, and a single-arm Mav2 control, respectively, with EGFR x RSV used as a baseline for comparison. For internalization assays, H1975 cells were plated into 96-well, clear culture plates at a density of 7×10³ cells per well. After approximately 16 hours of culture, test antibodies were mixed with rehydrated pH Antibody Labeling Reagent at a 1:3 molar ratio of test antibody to antibody labeling reagent for 15 min at 37° C. Labeled antibodies were dispensed onto cells. All the internalization assays were performed at a single concentration. Plates were placed into the Incucyte® Live-Cell Analysis System where images were acquired. Sampling of internalization images were taken at 0 minutes and at 45 minutes intervals over 72 hours. Image analysis was performed by using Incucyte's Base Analysis software. The “Top-Hat” background subtraction method was used to subtract background to give percent “Red Object Intensity”. Antibodies that had one domain that binds to a target and another domain that does not bind to a protein on the target cells were used as a baseline (single arm target antibodies).

The bispecific antibodies with both a target binding domain and a degrader protein binding domain resulted in higher rates of internalization than antibodies that bound only to internalizing proteins (FIG. 5). The internalization rate of the bispecific antibodies was also higher than the combined internalization rates of antibodies that bound only to degrader proteins and control antibodies that bound only to target proteins. These observations demonstrate that there is synergistic internalization activity for antibodies that bind both a target protein and a degrader protein on the target cells.

### Example 11—EGFR Internalization Screen to Identify Effective Degrader Protein Binding Domains for EGFR-Targeting Bispecific Antibodies

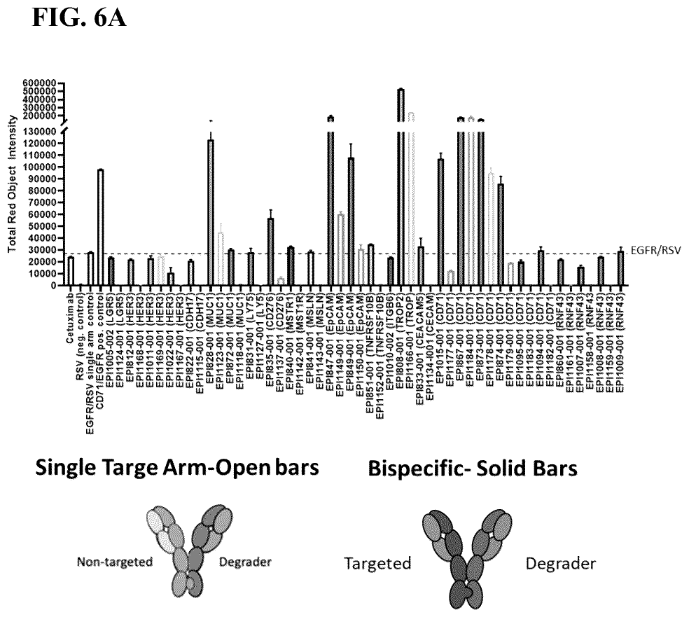

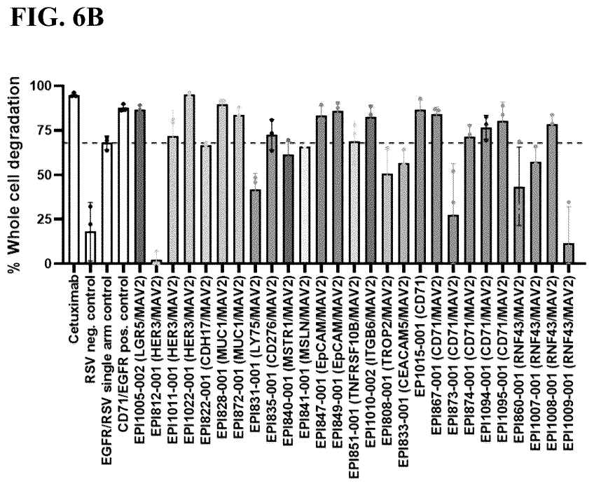

To further screen and validate effective degrader protein binding domains identified in the cell surface removal screen, an EGFR internalization assay was performed using 52 bispecifics (FIG. 6A). The 52 bispecific antibodies bound to EGFR as the target protein and 15 unique degrader proteins. For previously identified degrader proteins, multiple binding domains that bind to different epitopes were tested. Cetuximab, an IgGI isotype control, and EGFR x RSV were tested for comparison. The screen was performed in NCIH1975 (non-small cell lung cancer) cells using the methods described in Example 12. Results of screen identified degrader protein groups and specific molecular epitopes that, when paired with EGFR in a bispecific antibody format, demonstrated improved EGFR internalization as compared to palivizumab/Mav2, a bispecific pairing EGFR with a non-targeting control arm. In this example, Cetuximab corresponds to EPI431(Cetuximab commercial (MedChemExpress)), RSV neg. control corresponds to EP1692, EGFR/RSV single arm corresponds to EPI733, and CD71.EGFR corresponds to EPI259. The degrader proteins identified as efficacious include RNF43, MST1R, CD276, EpCAM, LGR5, ITGB6, TROP2, MUC1, and CD71. Four of these hits (CD276, MUC1, CD71, and EpCAM) showed synergistic internalization activity, three out of four of which were also identified in the cell surface removal assay. This demonstrates consistency amongst assays and that EGFR bispecific antibodies can cause synergistic internalization through identifiable degrader protein binding domains.

### Example 12—Whole Cell Degradation Screen to Identify Effective Degrader Protein Binding Domains for EGFR-Targeting Bispecific Antibodies

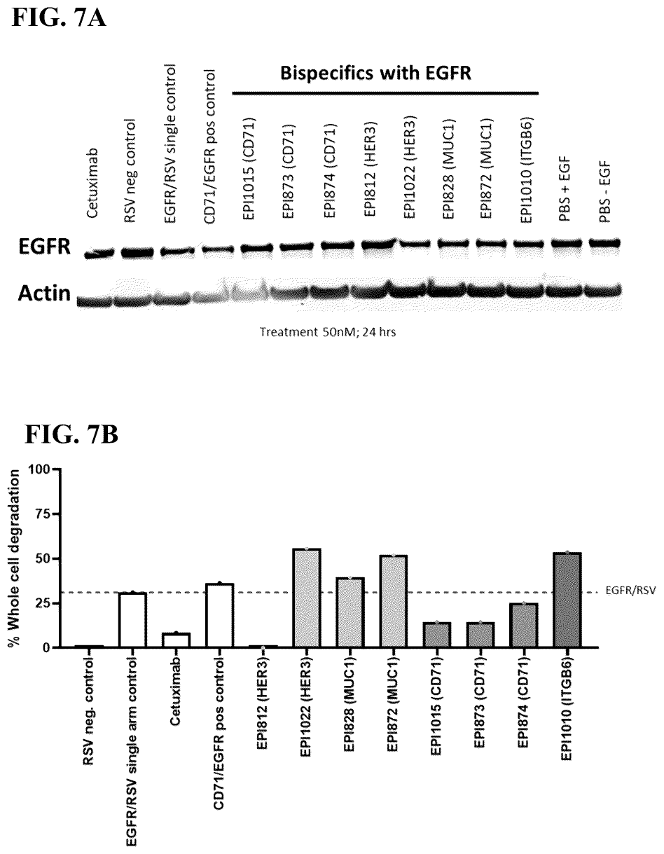

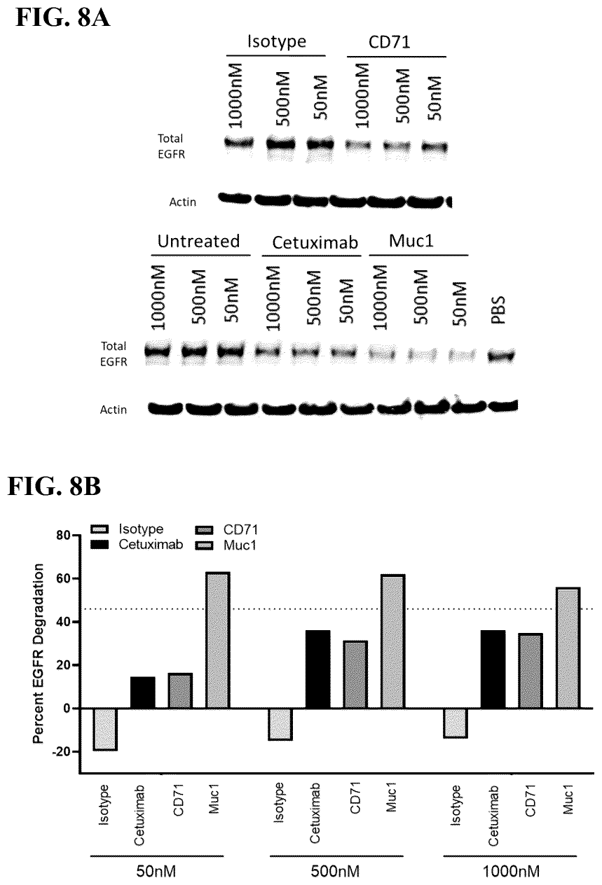

To measure degradation of the target protein, whole cell degradation of EGFR was tested using an AlphaLISA assay and western blots. In this example, 27 bispecifics which bound to 15 unique degrader proteins were screened using this assay (FIG. 6B). For previously identified degrader proteins, multiple binding domains that bind to different epitopes were tested. Cetuximab, an IgGI isotype control, and EGFR x RSV were tested for comparison. In the AlphaLISA assay, NCIH1975 cells were seeded in 384-well plate in reduced serum media. After approximately 16 hours of culture, a single concentration of antibodies was added to cells in reduced serum media and treated for 48 hours. Media was removed and stimulated with EGF in serum free media. Media was removed and cells were lysed. AlphaLISA acceptor beads and biotinylated antibodies were added to the lysate and incubated for 1 hour at room temperature. AlphaLISA donor beads were added to the lysate and incubated for 2 hours at room temperature. The plate was read on Perkin Elmer Envision to determine total EGFR levels. The results of the screen identified degrader protein groups and specific molecular epitopes that, when paired with EGFR in a bispecific antibody format, demonstrated improved whole cell degradation of EGFR as compared to Palivizumab x Mav2, a bispecific pairing EGFR with a non-targeting control arm. The degrader protein identified as efficacious in inducing EGFR degradation include CD276, LGR5, ITGB6, CD71, MUC1, RNF43, HER3, and EpCAM. As a result of this screen, four bispecifics (CD276, RNF43, MUC1, and ITGB6) were prioritized for further evaluation. The 8 bispecific antibodies were further assessed for whole cell degradation using western blot (FIGS. 7A-7B). Cetuximab, an IgGI isotype control, and Palivizumab x Mav2 (RSV and EGFR binding arms) were tested for comparison. For the western blot, NCIH1975 cells were seeded at a density of 4e5 cells in a 6 well tissue culture plate. After approximately 16 hours of culture, a single concentration of antibodies (50 nM) was added to cells in serum-starved media and treated for 24 to 48 hours. Media was removed and stimulated with EGF in serum free media. Media was removed and cells were lysed. Prepared samples were loaded onto a 4-12% BisTris gel and transferred to a PVDF membrane. The membrane was probed with EGFR or the housekeeping gene β-actin (FIG. 7A). Data was quantified using Empiria studio and the percent degradation was normalized to β-actin and compared to PBS control (FIG. 7B). By western blot, four of the eight bispecific antibodies demonstrated improved whole cell degradation of EGFR as compared to EGFR x RSV, a bispecific pairing EGFR with a non-targeting control arm. Bispecifics with a MUC1 binding domain were also found to result in higher EGFR degradation than cetuximab at various concentrations of antibody (FIGS. 8A and 8B). Together, these results demonstrate that EGFR bispecific antibodies can cause increased EGFR degradation when coupled with various degrader protein binding domains. In this examples, Isotype corresponds to EPI1102, Cetuximab corresponds to EPI431, EGFR/CD71 corresponds to EPI1015, and EGFR/MUC1 corresponds to EPI828.

### Example 13—EGFR Degradation and Cancer Cell Death with EGFRxRNF43 Bispecifics

To determine whether bispecific antibodies could induce targeted EGFR protein degradation and tumor cell growth suppression through ubiquitination of EGFR, a bispecific antibody targeting EGFR and RNF43 was developed (FIG. 9B). The expression of RNF43 and EGFR at mRNA in various cell lines was examined by RNA-sequencing. The assay was ran at the Board Institute (MA, USA), and the data were stored as Cancer Cell Line Encyclopedia (Nature. 2019 May; 569(7757):503-508). The mRNA expression level was analyzed by using cBioPortal (Table 11).

TABLE 11
Expression of RNF43 and EGFR in various cell lines

| | RNF43 expression | EGFR expression |
|---|---|---|
| Cell Line | (relative z-score, log RNA Seq RPKM) | (relative z-score, log RNA Seq RPKM) |
| HPAFII | 1.5332 | 0.8747 |
| HT29 | 2.672 | 0.0133 |
| LS180 | 2.9458 | 0.4492 |
| LS513 | 2.962 | 0.148 |

To identify an RNF43 binding domains to target in these studies, HEK293T cells transiently expressing GFP tagged RNF43 were used to identify RNF43 specific binders (FIG. 9A). The HEK293T cells were transfected with GFP-tagged RNF43 expressing construct. The expression level was previously determined by detecting GFP expression under microscope observation. The cells were harvested as a suspension using cell scarpers for flow cytometry analysis 24 hours post-transfection. After centrifugation for 4 minutes at 1,200 rpm, the supernatant was discarded. Cells were resuspended in antibody containing FACS buffer (PBS, 0.5% BSA, 0.05% sodium azide) for 60 minutes on ice. The cells were then washed with FACS buffer for three times, followed by an incubation of a secondary staining antibody (Goat anti-Human IgG (H+L)-647) for 45 minutes on ice. Post-staining, cells were washed four times in FACS buffer and stained with live/dead dye. Flow cytometry was performed on a CyTEK Aurora Flow Cytometer. SSC, FSC, and RNF43+, and GFP+ profiles were analyzed by using Cytobank. Antibodies that specifically bound to RNF43-GFP+ cells and not GFP-, non-transfected, or empty plasmid GFP+ cells were selected and reconstructed into a bispecific format with an anti-EGFR binder.

Next, cancer cell lines with EGFR expression and RNF43 co-expression were selected and membrane expression was validated prior to degradation studies. The cancer cell lines used were HPAFII, HT29, LS180, and LS513 with RNF43 and EGFR expression. To validate RNF43 membrane expression on the cancer cell lines, cells were grown on cover glasses in 24-well plates until attachment, and were stained in RNF43 binders and E-Cadherin antibody containing 1% BSA for 1 hour on ice, followed by incubation with secondary antibodies for 45 minutes at room temperature. The cells were washed briefly with 1×PBS and fixed with 4% paraformaldehyde for 15 minutes. The stained cells were counterstained with ProLong Gold Antifade Mountant with the blue DNA stain DAPI and mounted on microscope slides (FIG. 9C).

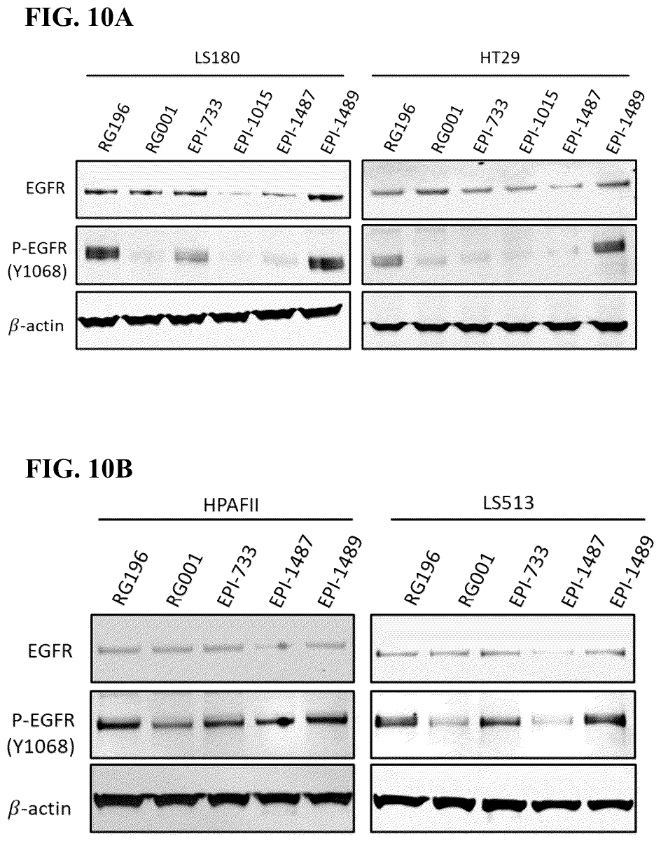

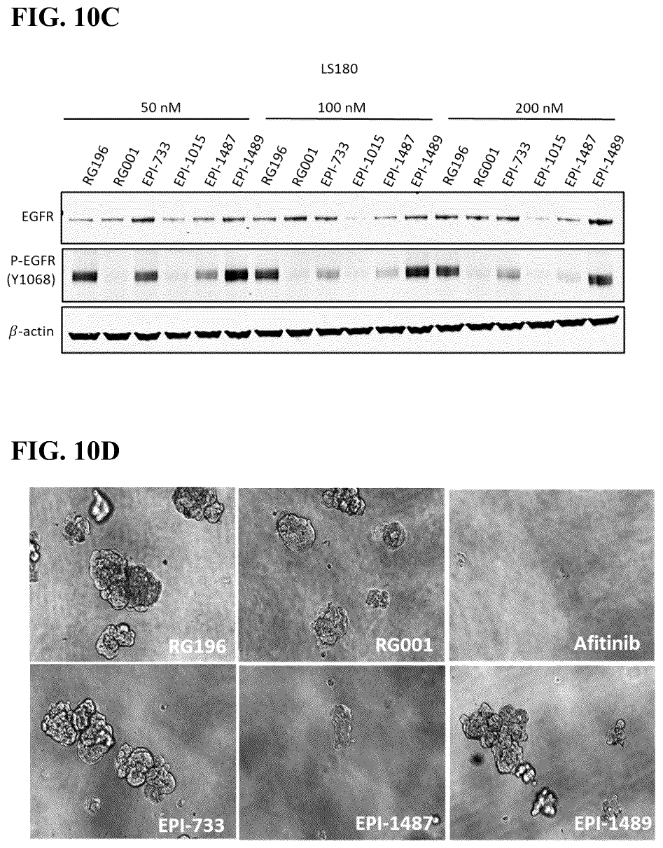

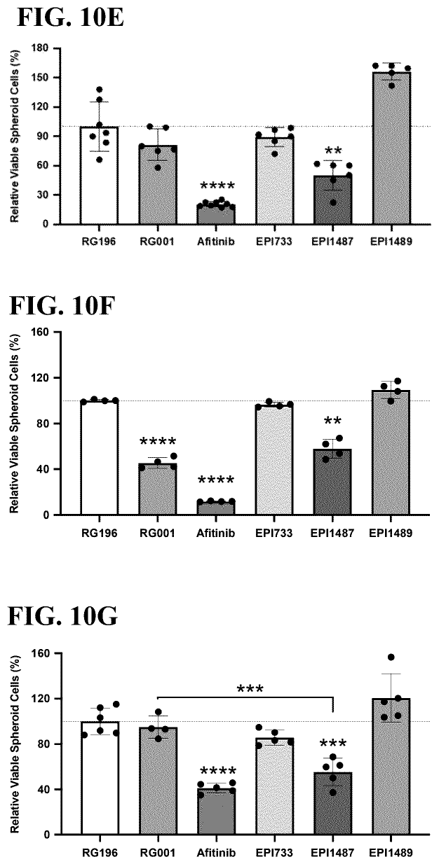

To characterize the EGFR degradation function of EGFR x RNF43 bispecific antibodies on tumor cells, degradation assays were performed. For these assays, HT29 and LS180 cells (FIG. 10A), and HPAFII and LS513 (FIG. 10B) cells were grown in two-dimensional culture conditions, treated with antibodies at a concentration of 200 nM for 48 hours, followed by western blot (Table 12).

TABLE 12
EGFR molecule information

| Molecule ID | Name | Arm 1 Binder | Arm 2 Binder |
|---|---|---|---|
| RG196 | Human IgG1 Isotype | | |
| | Control (BioXcell) | | |
| RG001 | Cetuximab (MCE) | EGFR | (IgG) |
| EPI733 | EGFR × EGFR | EGFR (Mav2) | RSV F |
| EPI1015 | EGFR × CD71 | EGFR (Mav2) | CD71 |
| EPI1487 | EGFR × RNF43 | EGFR (Mav2) | RNF43 |
| EPI1489 | RNF43 × RNF43 | EGFR (Mav2) | RNF43 |

For the western blot, cells were washed twice in ice-cold phosphate-buffered saline (PBS), lysed in 1% Triton lysis buffer (25 μM Tris [pH 7.5], 150 μM NaCl, 1% Triton X-100, 1 μM EDTA, 1 μM EGTA, 20 μM NaF, 1 μM Na2VO4, and 1 μM DTT) supplemented with a protease inhibitor cocktail (Roche) and cleared by centrifugation. Protein concentrations were determined by the Bio-Rad Protein Assay (BioRad). Equal amounts of protein extracts were resolved by SDS-PAGE (NuPAGE; Invitrogen), and proteins were transferred to a nitrocellulose or PVDF membrane. The membrane was immunoblotted with the indicated primary antibodies, incubated with secondary antibodies, and were either visualized with a LI-COR Odyssey scanner. The EGFR x RNF43 bispecific antibodies induced targeted EGFR degradation in all 4 tumor cell lines compared to cetuximab, the single armed EGFR binder, and single armed RNF43 binder (FIGS. 10A-10B). In some instances, as previously described, EGFR x CD71 bispecific was used as a positive control for comparison which also degraded EGFR to varying degrees in these cell lines. Phosphorylated EGFR was also degraded in these cells to varying degrees, confirming EGFR x RNF43 bispecific-mediated pharmacological inhibition on the proximal signaling event upstream of EGFR pathway signaling. The LS180 tumor cells, which had demonstrated robust EGFR x RNF43 bispecific-mediated EGFR degradation, were further investigated in a dose-response experiment (FIG. 10C). The strongest effect on EGFR and p-EGFR degradation was observed at 200 nM.

To characterize the cytolytic function of EGFRxRNF43 bispecific antibodies on tumor cell spheroid formation and growth, viability of 3D tumor spheroids was assessed after a two-week incubation with bispecific binders (FIGS. 10D-10G). Briefly, cells were harvested, counted, and seeded into Ultra-Low Attachment Culture 96-well plates. Two hundred cells were seeded in the wells of low-attachment 96-well plates in medium containing 10% Matrigel and bispecific binders at indicated doses. After a two-week incubation, an equal volume of CellTiter-Glo® 3D Cell Viability Assay reagent (Promega) was added and mixed by repeated pipetting to improve lysis of spheres. Plates were incubated for 30 minutes at room temperature while gently shaking on a rocker. Luminescence was measured using a microplate reader. Visual inspection by microscopy (representative images depicted in FIG. 10D) and functional assessment (FIGS. 10E-10G) by 3D CellTiter Glo assay indicated that EGFR x RNF43 bispecifics inhibited the formation and growth of tumor spheroids compared to single armed anti-EGFR, single armed anti-RNF43 mAb, and Afitinib . Additionally, EGFRxRNF43 bispecifics decreased spheroid formation and growth greater than cetuximab in LS180 and LS513 cells, but both had similar pharmacological effect on HPAFII cells. These data demonstrate that bispecific antibodies that bind to a target protein and a degrader protein result in degradation of the target protein and decreased viability of cancer cells expressing both the target protein and degrader protein.

### Example 14—Bispecifics that Bind to Target Protein and Degrader Protein Inhibit Tumor Growth and Induce Targeted Protein Degradation in Mice

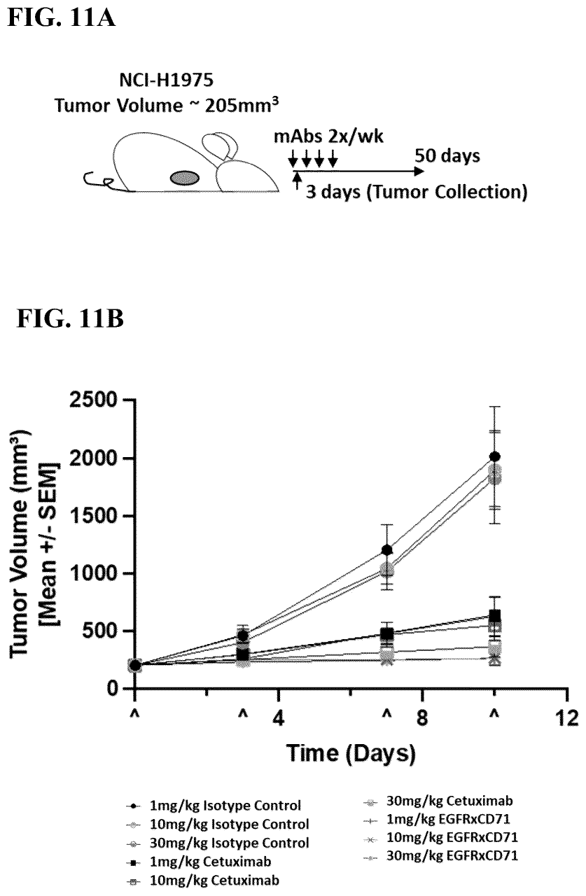

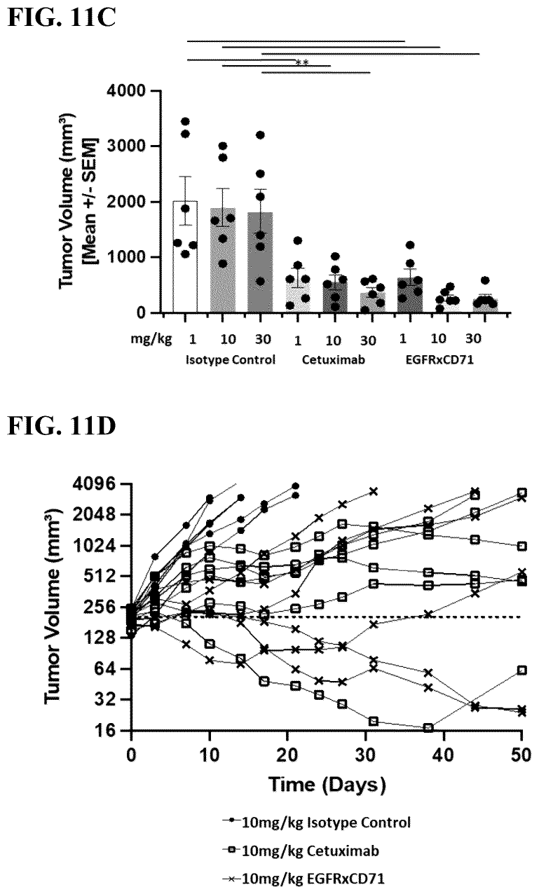

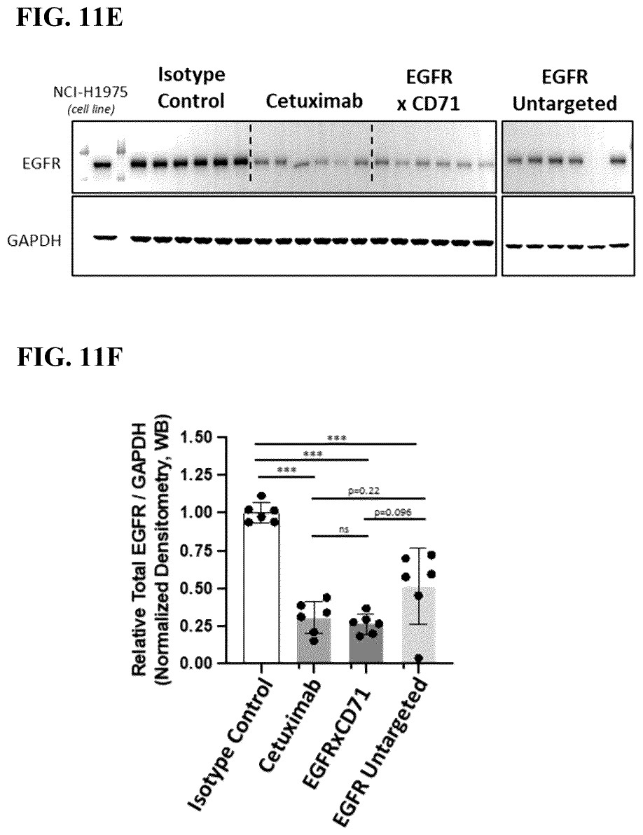

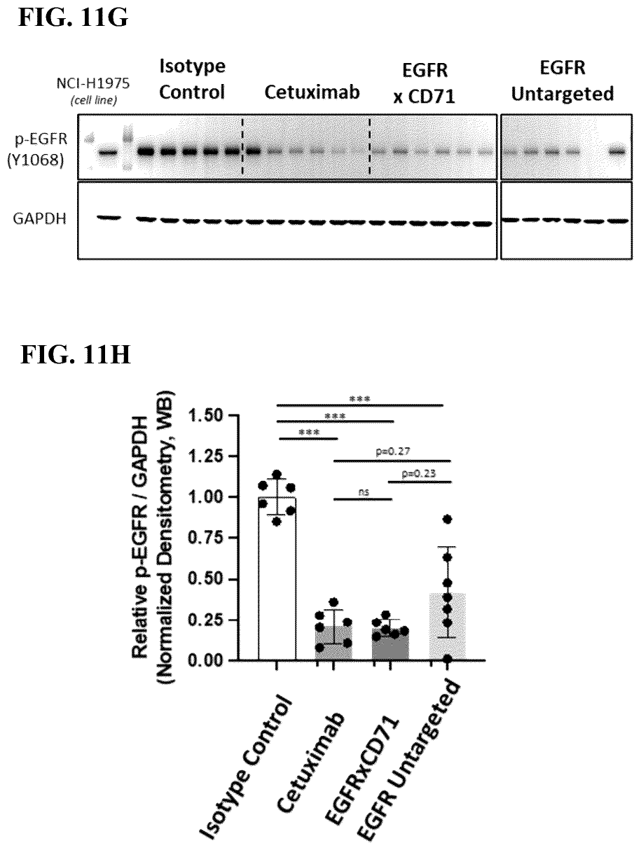

To determine if bispecific antibodies could pharmacologically inhibit tumor growth in mouse tumor models and induce targeted EGFR protein degradation in tumors when dosed systemically, an EGFR x CD71 (EPI511) bispecific was used. The NCIH1975 cells lines were grown in tissue culture flasks containing RPMI1640 medium supplemented with 10% fetal bovine serum at 37° C. in an atmosphere of 5% CO₂. Cells were harvested during exponential growth phase, and 5×10⁶ total cells were inoculated into the right front flank of BALB/c nude mice. Inoculated mice were monitored daily and tumor volumes and body weights were measured twice per week in accordance with IACUC guidelines. Tumors were grown until reaching approximately a volume of 205 mm³, at which point animals were randomized into groups (n=6 mice per group) and given intraperitoneal doses of the indicated antibodies. Bispecific antibodies were prepared in-house, Cetuximab was purchased from MedChemExpress (HY-P9905), and isotype control mAbs were purchased from BioXcell (BP0297). Dosing continued twice-per-week for 2 weeks and animals were monitored for up to 50 days from the initial dose. A subset of animals were dosed at day 0 and sacrificed for frozen tumor collection at 72 hours following a single dose of mAb. A study design schematic measuring tumor growth kinetics and pharmacodynamic degradation activity of bispecific mAbs compared to standard of care mAbs using an NCI-H1975 xenograft tumor model is shown (FIG. 11A). Tumor volume was calculated as V=(L x W x W)/2. Graphs and statistical analysis were done in Graphpad Prism using ordinary one-way ANOVA w/Tukey's multiple comparisons test, *p<0.05, **p<0.01, ***p<0.001 (FIGS. 11B-11D).

For EGFRxCD71 bispecific antibodies, significant tumor growth inhibition was observed at 1, 10 and 30 mg/kg dose levels within 10 days of treatment initiation (FIG. 11B). Additionally, the 10 and 30 mg/kg dose levels of EGFR x CD71 (EPI511) bispecific resulted in lower tumor volume than cetuximab at the corresponding dose (FIG. 11C). The 10 mg/kg group was monitored after the final (4^{th}) dose of mAbs, and extended tumor suppression was seen over the course of 50 days for cetuximab and the EGFR x CD71 (EPI511) bispecific antibodies (FIG. 11D).

To assess EGFR degradation in the tumor, a separate cohort of animals was treated with a single dose of 10 mg/kg EGFRxCD71 bispecific or control mAbs (controls comprise Isotype control (EPI1102) and Cetuximab (EP1431)). These animals were sacrificed at 72 hours to collect tumors and measure intratumoral EGFR and p-EGFR expression by western blot to monitor targeted protein degradation (FIG. 11E). Briefly, tumors were collected and immediately frozen in liquid nitrogen. Protein lysates were prepared by mechanical homogenization using a tissue homogenizer in RIPA lysis buffer. Protein quantification was measured by Pierce BCA Protein Assay Kit of ThermoFisher according to manufacturer's instructions. Equal amounts of protein per animal were loaded and run on NUPAGE, 4-12% Bio-Tris Midi Gels, followed by PVDF membrane transfer. Membranes were blocked in TBST+5% milk, followed by primary antibody, followed by three washes in TBST, then by secondary antibody in TBST+5% milk, three washes in TBST, then detection by Odyssey Infrared Imager. Separate gels were run and measured for EGFR, p-EGFR and GAPDH for each animal. Quantification of protein was performed using ImageJ and relative protein measurements were performed by dividing the signal intensity in the EGFR lanes by the signal in the paired sample GAPDH lanes (FIG. 11F). A normalization factor equating to the arbitrary value of 1 was defined using the mean value of the isotype control group animals. Each gel included the same set of isotype control group animals, collectively normalized to 1, to normalize values across all gels. Graphs and statistical analysis were completed in Graphpad Prism using ordinary one-way ANOVA w/Tukey's multiple comparisons test, *p<0.05, **p<0.01, ***p<0.001.

Treatment with EGFR x CD71 bispecific antibodies resulted in lower relative amounts of total EGFR compared to GAPDH in the tumor cells as compared to the isotype control and the single arm EGFR targeted antibodies, indicating higher induced intratumoral EGFR protein degradation in samples treated with the bispecific antibody (FIGS. 11E and 11F). The EGFR x CD71 bispecific antibody also resulted in lower relative phospho-EGFR to GAPDH when compared to controls (FIGS. 11G and 11H). These results indicate that bispecific antibodies that bind to a target protein and a degrader protein on the surface of cancer cells inhibit tumor growth and induce EGFR degradation in vivo.

### Example 17—Pharmacokinetics Assessment of Bispecific Antibodies

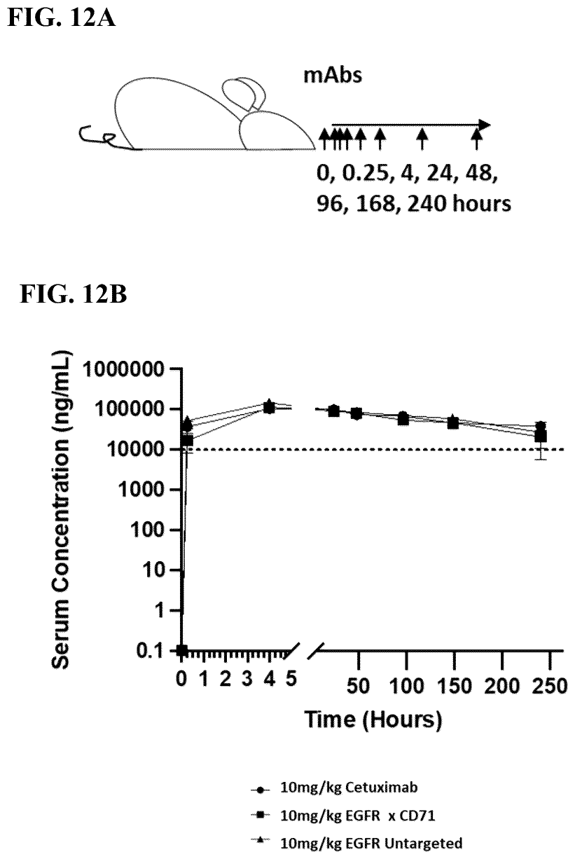

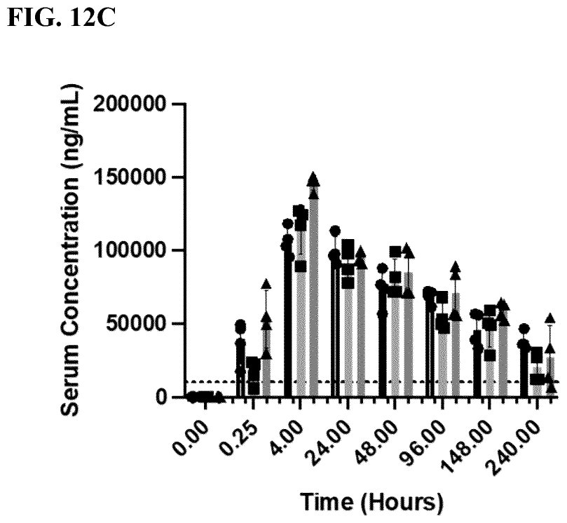

To determine pharmacokinetic properties of bispecific antibodies that bind to a target cancer-associated protein and a cancer-associated degrader protein in tumor-free mice, immunocompromised mice were randomized into groups and treated with EGFRxCD71 (EPI511) bispecific antibodies or control antibodies at a single 10 mg/kg dose level in the peritoneal cavity (FIG. 12A). Briefly, a cohort of 6-8 week old BALB/c nude mice were randomized intro groups (n=8/group) based on body weight, then injected with a single 5 uL/g volume dose of bispecific antibodies or Cetuximab at 10 mg/kg into the peritoneal cavity. Bispecific antibodies were prepared in-house and Cetuximab was purchased from MedChemExpress (HY-P9905). The initial dose was noted as timepoint=0. Serum samples were collected and frozen from each animal from the saphenous vein in the leg, per IACUC guidelines, at the following timepoints: Pre-dose, 0.25, 4, 24, 48, 96, 168, and 240 hours (FIGS. 12B and 12C). The collection of serum taken prior to dosing was used as a reference control. Sub-groups (n=4/group) were utilized to stagger blood collection from individual animals. Animals were monitored daily and weighed multiple times per week, according to IACUC guidelines. Serum concentration (ng/mL) of each mAb were measured using the Human Therapeutic IgGI ELISA Kit (Cayman #500910) according to the manufacturer's instructions. Concentrations of human IgGI in serum was computed relative to a standard curve of positive control samples. Pharmacokinetic analysis was performed using WinNonlin Phoenix software (Certara, version 8.2 or later). Graphs were created in Graphpad Prism on a log or linear scale. Dotted lines on the graph indicate 10,000 ng/mL for reference.

All treatment groups had measurable human IgG in serum at levels greater than 10 ug/mL at the 0.25 hour timepoint post-dose, and maximum concentration (Cmax) was achieved at 4 hours post-dose (FIG. 12B). By 24 hours, serum concentration began to decrease, but remained above 10 ug/mL throughout the 10 day study. Serum concentration from individual animals tracked closely within groups and between groups, with the greatest variation at the 0.25 hour (distribution) and 240 hour time points (FIG. 12C). Projected pharmacokinetic properties indicated that EGFR x CD71 bispecific antibodies, EGFR single arm controls, and Cetuximab had similar estimated half-lives (102.64, 122.71, 162.73 hours, respectively) and clearance rates (0.62, 0.48, and 0.42 mL/h/kg, respectively; Table 13).

TABLE 13
Pharmacokinetic properties of treatments in mice.

| Dose | | Estimated T_{1/2} | Estimated Clearance Rate |
|---|---|---|---|
| (mg/kg) | Treatment Group | (hours) | (mL/h/kg) |
| 10 | Cetuximab (EPI431) | 162.73 | 0.42 |
| 10 | EGFR × CD71 | 102.64 | 0.62 |
| | (EPI511) | | |
| 10 | EGFR Untargeted | 122.71 | 0.48 |
| | (EPI430) | | |

These data indicate that bispecific antibodies that bind to a target protein and a degrader protein have similar pharmacokinetics as standard of care antibodies.

### Additional Embodiments

Embodiment 1: A method of degrading an EGFR protein on a target cell, the method comprising: contacting the EGFR protein and a membrane-associated internalizing protein on the target cell with a bispecific binding agent, wherein the contacting of the EGFR protein and the membrane-associated internalizing protein with the bispecific binding agent leads to internalization and degradation of the EGFR protein; and wherein the bispecific binding agent comprises: (a) a first binding domain that specifically binds to an extracellular epitope the membrane associated internalizing protein; and (b) a second binding domain that specifically binds to an extracellular epitope on the EGFR protein; wherein the membrane associated internalizing protein is selected from CEACAM5, CEACAM6, HER3, MUC1, CD205, CD166, PRLR, SLC34A2, ITGB6, LRRC15, MUC16, SLC39A6, AXL, CD40, CD228, MUC5A, ITGB1, STn, KAAG1, DLK1, 5T4, SEZ6, ADAM9, I-AG7, ENPP3, CD46, CD56, ROR1, GPR20, TM4SF1, B7-H4, ALPP, LY6E, CLDN18, LY6G6D, GPR56, CD71, RNF43, RNF128, CD276, and CDH17.

Embodiment 2: The method of embodiment 1, wherein the membrane associated internalizing protein is selected from CD205, CD166, SLC34A2, ITGB6, LRRC15, MUC16, SLC39A6, AXL, CD40, CD228, MUC5A, ITGB1, STn, KAAG1, DLK1, 5T4, SEZ6, ADAM9, I-AG7, ENPP3, CD46, CD56, ROR1, GPR20, TM4SF1, B7-H4, ALPP, LY6E, CLDN18, LY6G6D, GPR56, and CD71.

Embodiment 3: The method of embodiment 1 or embodiment 2, wherein the membrane associated internalizing protein is CEACAM5.

Embodiment 4: The method of embodiment 1 or embodiment 2, wherein the membrane associated internalizing protein is CEACAM6.

Embodiment 5: The method of embodiment 1 or embodiment 2, wherein the membrane associated internalizing protein is HER3.

Embodiment 6: The method of embodiment 1 or embodiment 2, wherein the membrane associated internalizing protein is MUC1.

Embodiment 7: The method of embodiment 1 or embodiment 2, wherein the membrane associated internalizing protein is CD205.

Embodiment 8: The method of embodiment 1 or embodiment 2, wherein the membrane associated internalizing protein is CD166.

Embodiment 9: The method of embodiment 1 or embodiment 2, wherein the membrane associated internalizing protein is PRLR.

Embodiment 10: The method of embodiment 1 or embodiment 2, wherein the membrane associated internalizing protein is SLC34A2.

Embodiment 11: The method of embodiment 1 or embodiment 2, wherein the membrane associated internalizing protein is ITGB6.

Embodiment 12: The method of embodiment 1 or embodiment 2, wherein the membrane associated internalizing protein is LRRC15.

Embodiment 13: The method of embodiment 1 or embodiment 2, wherein the membrane associated internalizing protein is MUC16.

Embodiment 14: The method of any one of embodiments 1 to 13, wherein the bispecific binding agent comprises an antibody or portion thereof.

Embodiment 15: The method of any one of embodiments 1 to 13, wherein the bispecific binding agent comprises a bispecific antibody or portion thereof.

Embodiment 16: The method of any one of embodiments 1 to 13, wherein the bispecific binding agent comprises a knob and hole bispecific IgG.

Embodiment 17: The method of any one of embodiments 1 to 13, wherein the bispecific binding agent does not comprise an antibody-drug conjugate.

Embodiment 18: A bispecific binding agent comprising a bispecific antibody or antibody derivative, the bispecific binding agent comprising: a) a first binding domain that specifically binds to an extracellular epitope of an EGFR protein of a target cell; and b) a second binding domain that specifically binds to an extracellular epitope of a membrane-associated internalizing protein on a target cell; wherein the membrane associated internalizing protein is selected from CD205, CD166, SLC34A2, ITGB6, LRRC15, and MUC16 SLC39A6, AXL, CD40, CD228, MUC5A, ITGB1, STn, KAAG1, DLK1, 5T4, SEZ6, ADAM9, I-AG7, ENPP3, CD46, CD56, ROR1, GPR20, TM4SF1, B7-H4, ALPP, LY6E, CLDN18, LY6G6D, GPR56, CD71, RNF43, RNF128, CD276, and CDH17.

Embodiment 19: The bispecific binding agent of embodiment 18, wherein the membrane associated internalizing protein is CD205.

Embodiment 20: The bispecific binding agent of embodiment 18, wherein the membrane associated internalizing protein is CD166.

Embodiment 21: The bispecific binding agent of embodiment 18, wherein the membrane associated internalizing protein is SLC34A2.

Embodiment 22: The bispecific binding agent of embodiment 18, wherein membrane associated internalizing protein is ITGB6.

Embodiment 23: The bispecific binding agent of embodiment 18, wherein membrane associated internalizing protein is LRRC15.

Embodiment 24: The bispecific binding agent of embodiment 18, wherein the membrane associated internalizing protein is MUC16.

Embodiment 25: The bispecific binding agent of any one of embodiments 18 to 24, wherein the bispecific binding agent comprises a knob and hole bispecific IgG.

Embodiment 26: The bispecific binding agent of any one of embodiments 18 to 25, wherein the bispecific binding agent does not comprise an antibody-drug conjugate.

Embodiment 27: A pharmaceutical composition comprising a bispecific binding agent of agent of any one of embodiments 18 to 26 and a pharmaceutically acceptable excipient.

Embodiment 28: A method of treating cancer in a subject in need thereof, the method comprising administering to the subject a bispecific binding agent of any one of embodiments 18 to 26 or a pharmaceutical composition of embodiment 27.

Embodiment 29: A method of arresting growth of a target cell, the method comprising contacting the cell with a bispecific binding agent of any one of embodiments 18 to 26 or a pharmaceutical composition of embodiment 27.

Embodiment 30: The method of embodiment 29, wherein the cell is a cancer cell.

1. A method of degrading a target protein on a surface of a target cell, the method comprising:
contacting an endogenous internalizing receptor and the target protein on the surface of the target cell with an antibody, wherein the antibody comprises:
i. a first binding domain that specifically binds to an endogenous internalizing receptor, wherein the endogenous internalizing receptor is CDH17; and
ii. a second binding domain that specifically binds to the target protein, wherein the target protein is EGFR.

2-385. (canceled)

386. The method of claim 1, wherein the endogenous internalizing receptor is recycled to the target cell surface following the internalization of the antibody.

387. The method of claim 1, wherein the endogenous internalizing receptor is degraded.

388. The method of claim 1, wherein the target cell is a cancer cell.

389. The method of claim 1, wherein the target cell is selected from the group consisting of a breast cancer cell, a B cell lymphoma cell, a pancreatic cancer cell, a Hodgkin's lymphoma cell, an ovarian cancer cell, a prostate cancer cell, a mesothelioma cell, a lung cancer cell, a non-Hodgkin's B-cell (B-NHL) cell, a melanoma cell, a chronic lymphocytic leukemia cell, an acute lymphocytic leukemia cell, a neuroblastoma cell, a glioma cell, a glioblastoma cell, a bladder cancer cell, a colorectal cancer cell, and a head and neck cancer cell.

390. The method of claim 1, wherein expression of EGFR on the target cell decreases following contact with the antibody, as compared to a control target cell that is not contacted with the antibody.

391. The method of claim 1, wherein expression of EGFR on the target cell decreases by 50% or more following contact with the antibody relative to expression of EGFR on a control target cell not contacted with the antibody.

392. The method of claim 1, wherein expression of EGFR on the target cell decreases by 50% or more following contact with the antibody relative to expression of EGFR on a control target cell contacted with a monospecific EGFR antibody.

393. The method of claim 1, wherein cell surface removal of EGFR on the target cell is at least 20% or more following contact with the antibody relative to EGFR on a control target cell not contacted with the antibody.

394. The method of claim 1, wherein cell surface removal of EGFR on the target cell is at least 20% or more following contact with the antibody relative to EGFR on a control target cell contacted with a monospecific EGFR antibody.

395. The method of claim 1, wherein internalization of EGFR in the target cell is at least 20% or more following contact with the antibody relative to internalizing of EGFR in a control target cell not contacted with the antibody or contacted with a monospecific EGFR antibody.

396. The method of claim 1, wherein degradation of EGFR in the target cell is at least 20% or more following contact with the antibody relative to degradation of EGFR in a control target cell not contacted with the antibody or contacted with a monospecific EGFR antibody.

397. The method of claim 1, wherein a binding affinity of the antibody to EGFR is less than a binding affinity of Cetuximab to EGFR, and wherein the binding affinitay is measured by the Kd.

398. The method of claim 1, wherein the antibody is a bispecific antibody.

399. An antibody comprising:
a) a first binding domain that specifically binds to an endogenous internalizing receptor, wherein the endogenous internalizing receptor CDH17; and
b) a second binding domain that specifically binds to a target protein, wherein the target protein is EGFR.

400. A pharmaceutical composition comprising an antibody comprising:
a) a first binding domain that specifically binds to endogenous internalizing receptor, wherein the endogenous internalizing receptor is CDH17; and
b) a second binding domain that specifically binds to a target protein, wherein the target protein is EGFR.

401. A method comprising:
selecting a subject with tumor expressing EGFR and an endogenous internalizing receptor, wherein the endogenous internalizing receptor is CDH17; and
administering to said subject an antibody comprising:
a) a first binding domain that specifically binds to the endogenous internalizing receptor; and
b) a second binding domain that specifically binds to EGFR.

402. The method of claim 401, wherein the volume of the tumor decreases by 20% or more after administration of said antibody relative to the volume of a tumor not contacted with the antibody.

403. The method of claim 401, wherein the volume of the tumor is 80% or less after administration of said antibody relative to the volume of a tumor not contacted with the antibody.

404. A kit comprising an antibody comprising:
a) a first binding domain that specifically binds to endogenous internalizing receptor, wherein the endogenous internalizing receptor is CDH17; and
b) a second binding domain that specifically binds to a target protein, wherein the target protein is EGFR.


SEQUENCE LISTING (727 sequences)

SEQ ID NO: 1 - protein, 449 aa, synthetic construct
QVQLVQSGAEVKKPGASVKVSCETSGYTFTSYGISWVRQAPGHGLEWMGWISAYNGYTNYAQKLQGRVTMTTDTSTSTAYMELRSLRSDDTAVYYCARDLRGTNYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 119 aa, synthetic construct
QVQLVQSGAEVKKPGASVKVSCETSGYTFTSYGISWVRQAPGHGLEWMGWISAYNGYTNYAQKLQGRVTMTTDTSTSTAYMELRSLRSDDTAVYYCARDLRGTNYFDYWGQGTLVTVSS

SEQ ID NO: 3 - protein, 214 aa, synthetic construct
DIQMTQSPSSVSASVGDRVTITCRASQGISNWLAWFQHKPGKAPKLLIYAASSLLSGVPSRFSGSGSGTDFTLTISSLQPEDFATYYCQQANSFPITFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 4 - protein, 107 aa, synthetic construct
DIQMTQSPSSVSASVGDRVTITCRASQGISNWLAWFQHKPGKAPKLLIYAASSLLSGVPSRFSGSGSGTDFTLTISSLQPEDFATYYCQQANSFPITFGQGTRLEIK

SEQ ID NO: 5 - protein, 457 aa, synthetic construct
EVQLVQSGSKLKKPGASVKVSCKASGYTFTSYTMNWVRQAPGQGLEWMGWINTDTGDPTYAQGFTGRFVFSLDTSVSTAFLQINSLKAEDTAVYYCARGDCDSTSCYRYSYGYEDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 6 - protein, 127 aa, synthetic construct
EVQLVQSGSKLKKPGASVKVSCKASGYTFTSYTMNWVRQAPGQGLEWMGWINTDTGDPTYAQGFTGRFVFSLDTSVSTAFLQINSLKAEDTAVYYCARGDCDSTSCYRYSYGYEDYWGQGTLVTVSS

SEQ ID NO: 7 - protein, 214 aa, synthetic construct
DIQMTQSPSSLSASVGDRVTITCRASQSISSYLNWYQQKPGKAPKLLIYAASSLQSGVPSRFSGSGSGTDFTLTISSLQPEDFATYYCQQSYSTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 8 - protein, 107 aa, synthetic construct
DIQMTQSPSSLSASVGDRVTITCRASQSISSYLNWYQQKPGKAPKLLIYAASSLQSGVPSRFSGSGSGTDFTLTISSLQPEDFATYYCQQSYSTPPTFGQGTKVEIK

SEQ ID NO: 9 - protein, 459 aa, synthetic construct
QVTLRESGPALVKPTQTLTLTCTFSGFSLSTSGMSVGWIRQPPGKALEWLADIWWDDKKDYNPSLKSRLTISKDTSKNQVVLKVTNMDPADTATYYCARSMITNWYFDVWGAGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGSHHHHHH

SEQ ID NO: 10 - protein, 120 aa, synthetic construct
QVTLRESGPALVKPTQTLTLTCTFSGFSLSTSGMSVGWIRQPPGKALEWLADIWWDDKKDYNPSLKSRLTISKDTSKNQVVLKVTNMDPADTATYYCARSMITNWYFDVWGAGTTVTVSS

SEQ ID NO: 11 - protein, 213 aa, synthetic construct
DIQMTQSPSTLSASVGDRVTITCKCQLSVGYMHWYQQKPGKAPKLLIYDTSKLASGVPSRFSGSGSGTEFTLTISSLQPDDFATYYCFQGSGYPFTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 12 - protein, 106 aa, synthetic construct
DIQMTQSPSTLSASVGDRVTITCKCQLSVGYMHWYQQKPGKAPKLLIYDTSKLASGVPSRFSGSGSGTEFTLTISSLQPDDFATYYCFQGSGYPFTFGGGTKLEIK

SEQ ID NO: 13 - protein, 236 aa, synthetic construct
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGSHHHHHH

SEQ ID NO: 14 - protein, 451 aa, synthetic construct
QVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYFDVWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 15 - protein, 121 aa, synthetic construct
QVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYFDVWGQGSLVTVSS

SEQ ID NO: 16 - protein, 214 aa, synthetic construct
DIQLTQSPSSLSASVGDRVSITCKASQDVSIAVAWYQQKPGKAPKLLIYSASYRYTGVPDRFSGSGSGTDFTLTISSLQPEDFAVYYCQQHYITPLTFGAGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 17 - protein, 107 aa, synthetic construct
DIQLTQSPSSLSASVGDRVSITCKASQDVSIAVAWYQQKPGKAPKLLIYSASYRYTGVPDRFSGSGSGTDFTLTISSLQPEDFAVYYCQQHYITPLTFGAGTKVEIK

SEQ ID NO: 18 - protein, 454 aa, synthetic construct
QVQLQESGPGLVKPSETLSLTCTVSGGSISSSNYYWGWIRQPPGKGLEWIGNIYYRGYTYYNPSLKSRVTISVDTSKKQFSLTLSSVTAADTAMYYCAREGSDYGDYVGAFDIWDQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 19 - protein, 124 aa, synthetic construct
QVQLQESGPGLVKPSETLSLTCTVSGGSISSSNYYWGWIRQPPGKGLEWIGNIYYRGYTYYNPSLKSRVTISVDTSKKQFSLTLSSVTAADTAMYYCAREGSDYGDYVGAFDIWDQGTMVTVSS

SEQ ID NO: 20 - protein, 214 aa, synthetic construct; identical to SEQ ID NO: 7

SEQ ID NO: 21 - protein, 107 aa, synthetic construct; identical to SEQ ID NO: 8

SEQ ID NO: 22 - protein, 443 aa, synthetic construct
EVQLVQSGAEVKKPGASVKVSCKASGYTFTTYTIHWVRQAPGQGLEWMGYINPRSGYTEYNQKFQDRVTMTRDTSTSTVYMELSSLRSEDTAVYYCARSYEFWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 23 - protein, 113 aa, synthetic construct
EVQLVQSGAEVKKPGASVKVSCKASGYTFTTYTIHWVRQAPGQGLEWMGYINPRSGYTEYNQKFQDRVTMTRDTSTSTVYMELSSLRSEDTAVYYCARSYEFWGQGTTVTVSS

SEQ ID NO: 24 - protein, 214 aa, synthetic construct
EIVMTQSPATLSVSPGERATLSCKASQNVGINVAWYQQKPGQAPRALIYSASYRYSGIPARFSGSGSGTEFTLTISSLQSEDFAVYYCHQYKTYPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 25 - protein, 107 aa, synthetic construct
EIVMTQSPATLSVSPGERATLSCKASQNVGINVAWYQQKPGQAPRALIYSASYRYSGIPARFSGSGSGTEFTLTISSLQSEDFAVYYCHQYKTYPYTFGGGTKLEIK

SEQ ID NO: 26 - protein, 451 aa, synthetic construct
AVQLVESGGGSVQPGRSMRLSCAASGFTFSNYDMTWVRQAPTKGLEWVASITSDGGSTYSRDSVKGRFTISRDNAKSTLYLQMDSLRSEDTATYYCTTDRGRYLPYYFDYWGQGVMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 27 - protein, 121 aa, synthetic construct
AVQLVESGGGSVQPGRSMRLSCAASGFTFSNYDMTWVRQAPTKGLEWVASITSDGGSTYSRDSVKGRFTISRDNAKSTLYLQMDSLRSEDTATYYCTTDRGRYLPYYFDYWGQGVMVTVSS

SEQ ID NO: 28 - protein, 218 aa, synthetic construct
DVVLTQTPVSLSVTVGDQASISCRSSQSLEYSDGYSYLEWYLQKPGQSPQLLIYEVSSRFSGVPDRFIGSGSGTDFTLKISRVEPEDLGVYYCFQAIHDPTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 29 - protein, 111 aa, synthetic construct
DVVLTQTPVSLSVTVGDQASISCRSSQSLEYSDGYSYLEWYLQKPGQSPQLLIYEVSSRFSGVPDRFIGSGSGTDFTLKISRVEPEDLGVYYCFQAIHDPTFGAGTKLELK

SEQ ID NO: 30 - protein, 447 aa, synthetic construct
EVQLLESGGGLVQPGGSLRLSCAASGFTFSSYAMSWVRQAPGKGLEWVSAINSQGKSTYYADSVKGRFTISRDNSKNTLYLQMNSLRAEDTAVYYCARWGDEGFDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 31 - protein, 117 aa, synthetic construct
EVQLLESGGGLVQPGGSLRLSCAASGFTFSSYAMSWVRQAPGKGLEWVSAINSQGKSTYYADSVKGRFTISRDNSKNTLYLQMNSLRAEDTAVYYCARWGDEGFDIWGQGTLVTVSS

SEQ ID NO: 32 - protein, 214 aa, synthetic construct
DIQMTQSPSSLSASVGDRVTITCRASQGISNWLAWYQQKPGKAPKLLIYGASSLQSGVPSRFSGSGSGTDFTLTISSLQPEDFATYYCQQYSSFPTTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 33 - protein, 107 aa, synthetic construct
DIQMTQSPSSLSASVGDRVTITCRASQGISNWLAWYQQKPGKAPKLLIYGASSLQSGVPSRFSGSGSGTDFTLTISSLQPEDFATYYCQQYSSFPTTFGQGTKVEIK

SEQ ID NO: 34 - protein, 446 aa, synthetic construct
EVQLVESGGGLVQPGGSLRLSCAASGFTFSSYAMSWVRQTPGKGLEWVAVIDSNGGSTYYPDTVKDRFTISRDNSKNTLYLQMNSLRAEDTAVYYCSSYTNLGAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 35 - protein, 116 aa, synthetic construct
EVQLVESGGGLVQPGGSLRLSCAASGFTFSSYAMSWVRQTPGKGLEWVAVIDSNGGSTYYPDTVKDRFTISRDNSKNTLYLQMNSLRAEDTAVYYCSSYTNLGAYWGQGTLVTVSA

SEQ ID NO: 36 - protein, 214 aa, synthetic construct
DIQMTQSPSSLSASVGDRVTITCRASQDISGYLNWLQQKPGGAIKRLIYTTSTLDSGVPKRFSGSGSGTDFTLTISSLQSEDFATYYCLQYASSPFTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 37 - protein, 107 aa, synthetic construct
DIQMTQSPSSLSASVGDRVTITCRASQDISGYLNWLQQKPGGAIKRLIYTTSTLDSGVPKRFSGSGSGTDFTLTISSLQSEDFATYYCLQYASSPFTFGGGTKVEIK

SEQ ID NO: 38 - protein, 447 aa, synthetic construct
QVQLVESGGGVVQPGRSLRLSCAASGFTFSDYYMYWVRQAPGKGLEWVASISFDGTYTYYTDRVKGRFTISRDNSKNTLYLQMNSLRAEDTAVYYCARDRPAWFPYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 39 - protein, 117 aa, synthetic construct
QVQLVESGGGVVQPGRSLRLSCAASGFTFSDYYMYWVRQAPGKGLEWVASISFDGTYTYYTDRVKGRFTISRDNSKNTLYLQMNSLRAEDTAVYYCARDRPAWFPYWGQGTLVTVSA

SEQ ID NO: 40 - protein, 219 aa, synthetic construct
DIVMTQTPLSLSVTPGQPASISCRSSQSIVHSNGNTYLEWYLQKPGQSPQLLIYKVSNRFSGVPDRFSGSGSGTDFTLKISRVEAEDVGVYYCFQGSHVPLTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 41 - protein, 112 aa, synthetic construct
DIVMTQTPLSLSVTPGQPASISCRSSQSIVHSNGNTYLEWYLQKPGQSPQLLIYKVSNRFSGVPDRFSGSGSGTDFTLKISRVEAEDVGVYYCFQGSHVPLTFGAGTKLELK

SEQ ID NO: 42 - protein, 451 aa, synthetic construct
EVQLLETGGGVVKPGGSLKLSCAASGFTFSNYGMSWVRQTPEKRLEWVAAINRDGGTTYYTDNVKGRFTISRDNAKNSLYLQMSSLRSEDTALYYCARQFLLWDGWYFDVWGAGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 43 - protein, 121 aa, synthetic construct
EVQLLETGGGVVKPGGSLKLSCAASGFTFSNYGMSWVRQTPEKRLEWVAAINRDGGTTYYTDNVKGRFTISRDNAKNSLYLQMSSLRSEDTALYYCARQFLLWDGWYFDVWGAGTTVTVSS

SEQ ID NO: 44 - protein, 218 aa, synthetic construct
DVVLTQTPLSLPVTLGDQASISCRSSQSLLHSNGNTYLHWYLLKPGQSPKLLIYKVSNRFSGVPDRFSGSGSGTDFTLKITRVEAEDLGVYFCSQSTHVLTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 45 - protein, 111 aa, synthetic construct
DVVLTQTPLSLPVTLGDQASISCRSSQSLLHSNGNTYLHWYLLKPGQSPKLLIYKVSNRFSGVPDRFSGSGSGTDFTLKITRVEAEDLGVYFCSQSTHVLTFGAGTKLELK

SEQ ID NO: 46 - protein, 453 aa, synthetic construct
EVQLQQSVAELVKPGASVKMSCKVSGYTLTDHTIHWMKQRPEQGLEWIGYIYPRDGITGYNEKFKGKATLTADTSSSTAYMQLNSLTSEDSAVYFCARWGYSYRNYAYYYDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 47 - protein, 123 aa, synthetic construct
EVQLQQSVAELVKPGASVKMSCKVSGYTLTDHTIHWMKQRPEQGLEWIGYIYPRDGITGYNEKFKGKATLTADTSSSTAYMQLNSLTSEDSAVYFCARWGYSYRNYAYYYDYWGQGTTLTVSS

SEQ ID NO: 48 - protein, 220 aa, synthetic construct
DIVMSQSPSSLAVSVGEKVTMSCKSSQSLLHSSNQKNYLAWYQQKPGQSPKVLIYWASTRESGVPDRFTGSGSGTDFTLTITSVKSEDLAVYYCQQYYSYPWTFGGGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 49 - protein, 113 aa, synthetic construct
DIVMSQSPSSLAVSVGEKVTMSCKSSQSLLHSSNQKNYLAWYQQKPGQSPKVLIYWASTRESGVPDRFTGSGSGTDFTLTITSVKSEDLAVYYCQQYYSYPWTFGGGTRLEIK

SEQ ID NO: 50 - protein, 453 aa, synthetic construct
QVQLVQSGAEVKKPGSSVKVSCKASGYTFSDHTIHWVRQAPGQGLEWMGYIYPRLGSTKYAEKFQGRVTITADKSTSTAYMELSSLRSEDTAVYYCARWGYYYGSSRYYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 51 - protein, 123 aa, synthetic construct
QVQLVQSGAEVKKPGSSVKVSCKASGYTFSDHTIHWVRQAPGQGLEWMGYIYPRLGSTKYAEKFQGRVTITADKSTSTAYMELSSLRSEDTAVYYCARWGYYYGSSRYYFDYWGQGTLVTVSS

SEQ ID NO: 52 - protein, 220 aa, synthetic construct
EIVMTQSPATLSVSPGERATLSCRASQSVLYSSNQKQYLAWYQQKPGQAPRLLIYGASTRETGIPARFSGSGSGTEFTLTISSLQSEDFAVYYCQQYYSYPWTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 53 - protein, 113 aa, synthetic construct
EIVMTQSPATLSVSPGERATLSCRASQSVLYSSNQKQYLAWYQQKPGQAPRLLIYGASTRETGIPARFSGSGSGTEFTLTISSLQSEDFAVYYCQQYYSYPWTFGQGTKLEIK

SEQ ID NO: 54 - protein, 447 aa, synthetic construct
EVQLVESGGGLVQPGGSMRLSCVASGFPFSNYWMNWVRQAPGKGLEWVGEIRLKSNQYTTHYAESVKGRFTISRDDSKNSLYLQMNSLKTEDTAVYYCTRHYYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 55 - protein, 117 aa, synthetic construct
EVQLVESGGGLVQPGGSMRLSCVASGFPFSNYWMNWVRQAPGKGLEWVGEIRLKSNQYTTHYAESVKGRFTISRDDSKNSLYLQMNSLKTEDTAVYYCTRHYYFDYWGQGTLVTVSS

SEQ ID NO: 56 - protein, 219 aa, synthetic construct
DIVMTQSPLSNPVTPGEPASISCRSSKSLLHSNGITYFFWYLQKPGQSPQLLIYQMSNLASGVPDRFSGSGSGTDFTLRISRVEAEDVGVYYCAQNLELPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 57 - protein, 112 aa, synthetic construct
DIVMTQSPLSNPVTPGEPASISCRSSKSLLHSNGITYFFWYLQKPGQSPQLLIYQMSNLASGVPDRFSGSGSGTDFTLRISRVEAEDVGVYYCAQNLELPPTFGQGTKVEIK

SEQ ID NO: 58 - protein, 449 aa, synthetic construct
QVQLQQSGAEVKKPGASVKVSCEASGYTFPSYVLHWVKQAPGQGLEWIGYINPYNDGTQYNEKFKGKATLTRDTSINTAYMELSRLRSDDTAVYYCARGFGGSYGFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 59 - protein, 119 aa, synthetic construct
QVQLQQSGAEVKKPGASVKVSCEASGYTFPSYVLHWVKQAPGQGLEWIGYINPYNDGTQYNEKFKGKATLTRDTSINTAYMELSRLRSDDTAVYYCARGFGGSYGFAYWGQGTLVTVSS

SEQ ID NO: 60 - protein, 215 aa, synthetic construct
DIQLTQSPSSLSASVGDRVTMTCSASSSVSSSYLYWYQQKPGKAPKLWIYSTSNLASGVPARFSGSGSGTDFTLTISSLQPEDSASYFCHQWNRYPYTFGGGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 61 - protein, 108 aa, synthetic construct
DIQLTQSPSSLSASVGDRVTMTCSASSSVSSSYLYWYQQKPGKAPKLWIYSTSNLASGVPARFSGSGSGTDFTLTISSLQPEDSASYFCHQWNRYPYTFGGGTRLEIK

SEQ ID NO: 62 - protein, 448 aa, synthetic construct
QVQLVQSGAEVKKPGASVKVSCKASGYTFSAYWIEWVRQAPGKGLEWVGEILPGSGNSRYNEKFKGRVTVTRDTSTNTAYMELSSLRSEDTAVYYCARSYDFAWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 63 - protein, 118 aa, synthetic construct
QVQLVQSGAEVKKPGASVKVSCKASGYTFSAYWIEWVRQAPGKGLEWVGEILPGSGNSRYNEKFKGRVTVTRDTSTNTAYMELSSLRSEDTAVYYCARSYDFAWFAYWGQGTLVTVSS

SEQ ID NO: 64 - protein, 220 aa, synthetic construct
DIQMTQSPSSLSASVGDRVTITCKSSQSLLYSSNQKIYLAWYQQKPGKAPKLLIYWASTRESGVPSRFSGSGSGTDFTFTISSLQPEDIATYYCQQYYRYPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 65 - protein, 113 aa, synthetic construct
DIQMTQSPSSLSASVGDRVTITCKSSQSLLYSSNQKIYLAWYQQKPGKAPKLLIYWASTRESGVPSRFSGSGSGTDFTFTISSLQPEDIATYYCQQYYRYPRTFGQGTKVEIK

SEQ ID NO: 66 - protein, 447 aa, synthetic construct
QAQLVQSGAEVVKPGASVKMSCKASGYTFTSYNMHWVKQTPGQGLEWIGYIYPGNGATNYNQKFQGKATLTADPSSSTAYMQISSLTSEDSAVYFCARGDSVPFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 67 - protein, 117 aa, synthetic construct
QAQLVQSGAEVVKPGASVKMSCKASGYTFTSYNMHWVKQTPGQGLEWIGYIYPGNGATNYNQKFQGKATLTADPSSSTAYMQISSLTSEDSAVYFCARGDSVPFAYWGQGTLVTVSA

SEQ ID NO: 68 - protein, 213 aa, synthetic construct
EIVLTQSPATMSASPGERVTITCSAHSSVSFMHWFQQKPGTSPKLWIYSTSSLASGVPARFGGSGSGTSYSLTISSMEAEDAATYYCQQRSSFPLTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 69 - protein, 106 aa, synthetic construct
EIVLTQSPATMSASPGERVTITCSAHSSVSFMHWFQQKPGTSPKLWIYSTSSLASGVPARFGGSGSGTSYSLTISSMEAEDAATYYCQQRSSFPLTFGAGTKLELK

SEQ ID NO: 70 - protein, 446 aa, synthetic construct
EVKLVESGGGLVAPGGSLKLSCAASGFTFSSYPMSWVRQTPEKRLEWVAYINNGGGNPYYPDTVKGRFTISRDNAKNTLYLQMSSLKSEDTAIYYCIRQYYGFDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 71 - protein, 116 aa, synthetic construct
EVKLVESGGGLVAPGGSLKLSCAASGFTFSSYPMSWVRQTPEKRLEWVAYINNGGGNPYYPDTVKGRFTISRDNAKNTLYLQMSSLKSEDTAIYYCIRQYYGFDYWGQGTTLTVSS

SEQ ID NO: 72 - protein, 219 aa, synthetic construct
DVLMTQTPLSLPVSLGDQASISCRSSQTIVHSNGKIYLEWYLQKPGQSPKLLIYRVSKRFSGVPDRFSGSGSGTDFTLKISRVEAEDLGVYYCFQGSHVPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 73 - protein, 112 aa, synthetic construct
DVLMTQTPLSLPVSLGDQASISCRSSQTIVHSNGKIYLEWYLQKPGQSPKLLIYRVSKRFSGVPDRFSGSGSGTDFTLKISRVEAEDLGVYYCFQGSHVPWTFGGGTKLEIK

SEQ ID NO: 74 - protein, 448 aa, synthetic construct
QVQLVESGGGVVQPGRSLRLSCAASGFTFSNYGMYWVRQAPGKGLEWVAVIWYDGSNKYYADSVKGRFTISRDNSKNTLYLQMNSLRAEDTAVYYCARDLWGWYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 75 - protein, 118 aa, synthetic construct
QVQLVESGGGVVQPGRSLRLSCAASGFTFSNYGMYWVRQAPGKGLEWVAVIWYDGSNKYYADSVKGRFTISRDNSKNTLYLQMNSLRAEDTAVYYCARDLWGWYFDYWGQGTLVTVSS

SEQ ID NO: 76 - protein, 214 aa, synthetic construct
EIVLTQSPATLSLSPGERATLSCRASQSVSSYLAWYQQKPGQAPRLLIYDASNRATGIPARFSGSGSGTDFTLTISSLEPEDFAVYYCQQRRNWPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 77 - protein, 107 aa, synthetic construct
EIVLTQSPATLSLSPGERATLSCRASQSVSSYLAWYQQKPGQAPRLLIYDASNRATGIPARFSGSGSGTDFTLTISSLEPEDFAVYYCQQRRNWPLTFGGGTKVEIK

SEQ ID NO: 78 - protein, 445 aa, synthetic construct
QVQLVESGGGVVQPGRSLRLSCAASGFIFSIYGMHWVRQAPGKGLEWVAVIWYDGSNKYYADSVKGRFTISRDNSKNTLYLQMNSLRAEDTAVYYCARAPHFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 79 - protein, 115 aa, synthetic construct
QVQLVESGGGVVQPGRSLRLSCAASGFIFSIYGMHWVRQAPGKGLEWVAVIWYDGSNKYYADSVKGRFTISRDNSKNTLYLQMNSLRAEDTAVYYCARAPHFDYWGQGTLVTVSS

SEQ ID NO: 80 - protein, 214 aa, synthetic construct
DIQMTQSPSSLSASVGDRVTITCRASQGISSWLAWYQQKPEKAPKSLIYAASSLQSGVPSRFSGSGSGTDFTLTISSLQPEDFATYYCQQYNSYPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 81 - protein, 107 aa, synthetic construct
DIQMTQSPSSLSASVGDRVTITCRASQGISSWLAWYQQKPEKAPKSLIYAASSLQSGVPSRFSGSGSGTDFTLTISSLQPEDFATYYCQQYNSYPYTFGQGTKLEIK

SEQ ID NO: 82 - protein, 449 aa, synthetic construct
EVQLVESGGGLVKPGGSLRLSCAASGFTYSNAWMSWVRQAPGKGLEWVGRIKSKTDGGTTDYAAPVQGRFTISRDDSKNTLYLQMNSLKTEDTAVYYCTIFGVVSFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 83 - protein, 119 aa, synthetic construct
EVQLVESGGGLVKPGGSLRLSCAASGFTYSNAWMSWVRQAPGKGLEWVGRIKSKTDGGTTDYAAPVQGRFTISRDDSKNTLYLQMNSLKTEDTAVYYCTIFGVVSFDYWGQGTLVTVSS

SEQ ID NO: 84 - protein, 214 aa, synthetic construct
DVQMTQSPSSLSASVGDRVTITCRASQSISDYLSWYQQRPGKAPNLLIYAASNLKTGVPSRFSGSGSGTDFTLTISTLQPEDFATYYCQQSYRSPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 85 - protein, 107 aa, synthetic construct
DVQMTQSPSSLSASVGDRVTITCRASQSISDYLSWYQQRPGKAPNLLIYAASNLKTGVPSRFSGSGSGTDFTLTISTLQPEDFATYYCQQSYRSPWTFGQGTKVEIK

SEQ ID NO: 86 - protein, 451 aa, synthetic construct
QVQLVQSGAEVKKPGASVKVSCKASGYTFTEFGMNWVRQAPGQGLEWMGWINTKTGEATYVEEFKGRVTFTTDTSTSTAYMELRSLRSDDTAVYYCARWDFAYYVEAMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 87 - protein, 121 aa, synthetic construct
QVQLVQSGAEVKKPGASVKVSCKASGYTFTEFGMNWVRQAPGQGLEWMGWINTKTGEATYVEEFKGRVTFTTDTSTSTAYMELRSLRSDDTAVYYCARWDFAYYVEAMDYWGQGTTVTVSS

SEQ ID NO: 88 - protein, 215 aa, synthetic construct
DIQMTQSPSSLSASVGDRVTITCKASAAVGTYVAWYQQKPGKAPKLLIYSASYRKRGVPSRFSGSGSGTDFTLTISSLQPEDFATYYCHQYYTYPLFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 89 - protein, 108 aa, synthetic construct
DIQMTQSPSSLSASVGDRVTITCKASAAVGTYVAWYQQKPGKAPKLLIYSASYRKRGVPSRFSGSGSGTDFTLTISSLQPEDFATYYCHQYYTYPLFTFGQGTKLEIK

SEQ ID NO: 90 - protein, 451 aa, synthetic construct
EVKLVESGGGLVQPGGSLRLSCATSGFTFTDYYMNWVRQPPGKALEWLGFIGNKANGYTTEYSASVKGRFTISRDKSQSILYLQMNTLRAEDSATYYCTRDRGLRFYFDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 91 - protein, 121 aa, synthetic construct
EVKLVESGGGLVQPGGSLRLSCATSGFTFTDYYMNWVRQPPGKALEWLGFIGNKANGYTTEYSASVKGRFTISRDKSQSILYLQMNTLRAEDSATYYCTRDRGLRFYFDYWGQGTTLTVSS

SEQ ID NO: 92 - protein, 213 aa, synthetic construct
QTVLSQSPAILSASPGEKVTMTCRASSSVTYIHWYQQKPGSSPKSWIYATSNLASGVPARFSGSGSGTSYSLTISRVEAEDAATYYCQHWSSKPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 93 - protein, 106 aa, synthetic construct
QTVLSQSPAILSASPGEKVTMTCRASSSVTYIHWYQQKPGSSPKSWIYATSNLASGVPARFSGSGSGTSYSLTISRVEAEDAATYYCQHWSSKPPTFGGGTKLEIK

SEQ ID NO: 94 - protein, 448 aa, synthetic construct
QVQLQQSGAELVKPGASVKLSCKASGYTFTNYDINWVRQRPEQGLEWIGWIFPGDGSTQYNEKFKGKATLTTDTSSSTAYMQLSRLTSEDSAVYFCARQTTATWFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 95 - protein, 118 aa, synthetic construct
QVQLQQSGAELVKPGASVKLSCKASGYTFTNYDINWVRQRPEQGLEWIGWIFPGDGSTQYNEKFKGKATLTTDTSSSTAYMQLSRLTSEDSAVYFCARQTTATWFAYWGQGTLVTVSA

SEQ ID NO: 96 - protein, 214 aa, synthetic construct
DIVMTQSPATLSVTPGDRVSLSCRASQSISDYLHWYQQKSHESPRLLIKYASQSISGIPSRFSGSGSGSDFTLSINSVEPEDVGVYYCQNGHSFPLTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 97 - protein, 107 aa, synthetic construct
DIVMTQSPATLSVTPGDRVSLSCRASQSISDYLHWYQQKSHESPRLLIKYASQSISGIPSRFSGSGSGSDFTLSINSVEPEDVGVYYCQNGHSFPLTFGAGTKLELK

SEQ ID NO: 98 - protein, 446 aa, synthetic construct
EVQLQESGPGLVKPSETLSLTCAVTGYSITSGYSWHWIRQFPGNGLEWMGYIHSSGSTNYNPSLKSRISISRDTSKNQFFLKLSSVTAADTAVYYCAGYDDYFEYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 99 - protein, 116 aa, synthetic construct
EVQLQESGPGLVKPSETLSLTCAVTGYSITSGYSWHWIRQFPGNGLEWMGYIHSSGSTNYNPSLKSRISISRDTSKNQFFLKLSSVTAADTAVYYCAGYDDYFEYWGQGTTVTVSS

SEQ ID NO: 100 - protein, 214 aa, synthetic construct
DIQMTQSPSSLSASVGDRVTITCKASQNVGFNVAWYQQKPGKSPKALIYSASYRYSGVPSRFSGSGSGTDFTLTISSLQPEDFAEYFCQQYNWYPFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 101 - protein, 107 aa, synthetic construct
DIQMTQSPSSLSASVGDRVTITCKASQNVGFNVAWYQQKPGKSPKALIYSASYRYSGVPSRFSGSGSGTDFTLTISSLQPEDFAEYFCQQYNWYPFTFGQGTKLEIK

SEQ ID NO: 102 - protein, 452 aa, synthetic construct
QVQLVQSGAEVKKPGSSVKVSCKASGYTFTNYVMHWVRQAPGQGLEWMGYINPYNDDVKYNEKFKGRVTITADESTSTAYMELSSLRSEDTAVYYCARWGYYGSPLYYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 103 - protein, 122 aa, synthetic construct
QVQLVQSGAEVKKPGSSVKVSCKASGYTFTNYVMHWVRQAPGQGLEWMGYINPYNDDVKYNEKFKGRVTITADESTSTAYMELSSLRSEDTAVYYCARWGYYGSPLYYFDYWGQGTLVTVSS

SEQ ID NO: 104 - protein, 213 aa, synthetic construct
EIVLTQSPATLSLSPGERATLSCRASSRLIYMHWYQQKPGQAPRPLIYATSNLASGIPARFSGSGSGTDFTLTISSLEPEDFAVYYCQQWNSNPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 105 - protein, 106 aa, synthetic construct
EIVLTQSPATLSLSPGERATLSCRASSRLIYMHWYQQKPGQAPRPLIYATSNLASGIPARFSGSGSGTDFTLTISSLEPEDFAVYYCQQWNSNPPTFGQGTKVEIK

SEQ ID NO: 106 - protein, 452 aa, synthetic construct
EVQLVESGGGLVQPGGSLRLSCAASGFTFSSYLMTWVRQAPGKGLEWVANIKQDGSEKYYVDSVKGRFTISRDNAKNSLNLQMNSLRAEDTAVYYCTRDGYSSGRHYGMDVWGQGTTVIVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 107 - protein, 122 aa, synthetic construct
EVQLVESGGGLVQPGGSLRLSCAASGFTFSSYLMTWVRQAPGKGLEWVANIKQDGSEKYYVDSVKGRFTISRDNAKNSLNLQMNSLRAEDTAVYYCTRDGYSSGRHYGMDVWGQGTTVIVSS

SEQ ID NO: 108 - protein, 214 aa, synthetic construct
EIVLTQSPATLSLSPGERATLSCRASQSVSRYLAWYQQKPGQAPRLLIYDASNRATGIPARFSGSGSGTDFTLTISSLEPEDFAVYYCQQRSNWPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 109 - protein, 107 aa, synthetic construct
EIVLTQSPATLSLSPGERATLSCRASQSVSRYLAWYQQKPGQAPRLLIYDASNRATGIPARFSGSGSGTDFTLTISSLEPEDFAVYYCQQRSNWPRTFGQGTKVEIK

SEQ ID NO: 110 - protein, 451 aa, synthetic construct
EVQLVESGGGLVQPGGSLRLSCAASGFTFSRHWMSWVRQAPGKGLEWVSEINPDSRTINYAPSVKGRFTISRDNAKNSLYLQMNSLRAEDTAVYYCARRVRIHYYGAMDSWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 111 - protein, 121 aa, synthetic construct
EVQLVESGGGLVQPGGSLRLSCAASGFTFSRHWMSWVRQAPGKGLEWVSEINPDSRTINYAPSVKGRFTISRDNAKNSLYLQMNSLRAEDTAVYYCARRVRIHYYGAMDSWGQGTTVTVSS

SEQ ID NO: 112 - protein, 214 aa, synthetic construct
DIQLTQSQSFVSTSVGDRVTVTCRASQNVGSSLVWYQQKPGKSPKTLIYSASFLYSGVPSRFSGSGSGTEFTLTISSVQPEDFADYFCQQYNNYPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 113 - protein, 107 aa, synthetic construct
DIQLTQSQSFVSTSVGDRVTVTCRASQNVGSSLVWYQQKPGKSPKTLIYSASFLYSGVPSRFSGSGSGTEFTLTISSVQPEDFADYFCQQYNNYPLTFGGGTKVEIK

SEQ ID NO: 114 - protein, 449 aa, synthetic construct
EVQLQQSGAELVKPGASVKLSCTTSGFNIIDTYIHWVNQKPDQGLEWIGRIDPADGNRKSDPKFQVKATITVDTSSNTAYLQLSSLTSGDTAVYYCARGYGNLNAMDSWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 115 - protein, 119 aa, synthetic construct
EVQLQQSGAELVKPGASVKLSCTTSGFNIIDTYIHWVNQKPDQGLEWIGRIDPADGNRKSDPKFQVKATITVDTSSNTAYLQLSSLTSGDTAVYYCARGYGNLNAMDSWGQGTSVTVSS

SEQ ID NO: 116 - protein, 214 aa, synthetic construct
DIQMNQSPSSLSASLGDTITITCHASQNINVWLNWYQQKPGNIPKLLIYKASNLHTGVPSRFSGSGSGTGFTLTISSLQPEDIATYYCQQGQSYPLTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 117 - protein, 107 aa, synthetic construct
DIQMNQSPSSLSASLGDTITITCHASQNINVWLNWYQQKPGNIPKLLIYKASNLHTGVPSRFSGSGSGTGFTLTISSLQPEDIATYYCQQGQSYPLTFGGGTKLEIK

SEQ ID NO: 118 - protein, 452 aa, synthetic construct
QVQLVQSGAEVKKPGATVKISCKVSGYTFTDYHMDWVQQAPGKGLEWMGDINPNNGGAIYNQKFKGRVTITADTSTDTAYMELSSLRSEDTAVYYCARSHYDYAGGAWFAYWGQGTLVTVSRASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 119 - protein, 122 aa, synthetic construct
QVQLVQSGAEVKKPGATVKISCKVSGYTFTDYHMDWVQQAPGKGLEWMGDINPNNGGAIYNQKFKGRVTITADTSTDTAYMELSSLRSEDTAVYYCARSHYDYAGGAWFAYWGQGTLVTVSR

SEQ ID NO: 120 - protein, 220 aa, synthetic construct
EIVMTQSPGTLSLSPGERATLSCKSSQSLLFSGNQKNYLAWYQQKPGQAPRLLIYWASTRASGIPDRFSGSGSGTDFTLTISRLEPEDFAVYYCQQYYSFPRTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 121 - protein, 113 aa, synthetic construct
EIVMTQSPGTLSLSPGERATLSCKSSQSLLFSGNQKNYLAWYQQKPGQAPRLLIYWASTRASGIPDRFSGSGSGTDFTLTISRLEPEDFAVYYCQQYYSFPRTFGQGTKLEIK

SEQ ID NO: 122 - protein, 450 aa, synthetic construct
QVELVQSGAEVKKPGESLKISCKGSGYSFTSYWIGWVRQAPGKGLEWMGIIDPGDSRTRYSPSFQGQVTISADKSISTAYLQWSSLKASDTAMYYCARGQLYGGTYMDGWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 123 - protein, 120 aa, synthetic construct
QVELVQSGAEVKKPGESLKISCKGSGYSFTSYWIGWVRQAPGKGLEWMGIIDPGDSRTRYSPSFQGQVTISADKSISTAYLQWSSLKASDTAMYYCARGQLYGGTYMDGWGQGTLVTVSS

SEQ ID NO: 124 - protein, 218 aa, synthetic construct
DIALTQPASVSGSPGQSITISCTGTSSDIGGYNSVSWYQQHPGKAPKLMIYGVNNRPSGVSNRFSGSKSGNTASLTISGLQAEDEADYYCSSYDIESATPVFGGGTKLTVLRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 125 - protein, 111 aa, synthetic construct
DIALTQPASVSGSPGQSITISCTGTSSDIGGYNSVSWYQQHPGKAPKLMIYGVNNRPSGVSNRFSGSKSGNTASLTISGLQAEDEADYYCSSYDIESATPVFGGGTKLTVL

SEQ ID NO: 126 - protein, 449 aa, synthetic construct
QVQLQQSGPELEKPGASVKISCKASGYSFTGYTMNWVKQSHGKSLEWIGLITPYNGASSYNQKFRGKATLTVDKSSSTAYMDLLSLTSEDSAVYFCARGGYDGRGFDYWGSGTPVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 127 - protein, 119 aa, synthetic construct
QVQLQQSGPELEKPGASVKISCKASGYSFTGYTMNWVKQSHGKSLEWIGLITPYNGASSYNQKFRGKATLTVDKSSSTAYMDLLSLTSEDSAVYFCARGGYDGRGFDYWGSGTPVTVSS

SEQ ID NO: 128 - protein, 213 aa, synthetic construct
DIELTQSPAIMSASPGEKVTMTCSASSSVSYMHWYQQKSGTSPKRWIYDTSKLASGVPGRFSGSGSGNSYSLTISSVEAEDDATYYCQQWSKHPLTFGSGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 129 - protein, 106 aa, synthetic construct
DIELTQSPAIMSASPGEKVTMTCSASSSVSYMHWYQQKSGTSPKRWIYDTSKLASGVPGRFSGSGSGNSYSLTISSVEAEDDATYYCQQWSKHPLTFGSGTKVEIK

SEQ ID NO: 130 - protein, 452 aa, synthetic construct
QVHLVESGGGVVQPGRSLRLSCVASGITFRIYGMHWVRQAPGKGLEWVAVLWYDGSHEYYADSVKGRFTISRDNSKNTLYLQMNSLRAEDTAIYYCARDGDYYDSGSPLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 131 - protein, 122 aa, synthetic construct
QVHLVESGGGVVQPGRSLRLSCVASGITFRIYGMHWVRQAPGKGLEWVAVLWYDGSHEYYADSVKGRFTISRDNSKNTLYLQMNSLRAEDTAIYYCARDGDYYDSGSPLDYWGQGTLVTVSS

SEQ ID NO: 132 - protein, 214 aa, synthetic construct
EIVLTQSPATLSLSPGERATLSCRASQSVSSYLAWYQQKPGQAPRLLIYDASNRATGIPARFSGSGSGTDFTLTISSLEPEDFAVYYCQQRSNWPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 133 - protein, 107 aa, synthetic construct
EIVLTQSPATLSLSPGERATLSCRASQSVSSYLAWYQQKPGQAPRLLIYDASNRATGIPARFSGSGSGTDFTLTISSLEPEDFAVYYCQQRSNWPLTFGGGTKVEIK

SEQ ID NO: 134 - protein, 452 aa, synthetic construct
EVQLVQSGAEVKKPGASVKVSCKASGDTFKRYYVHWVRQAPGQGLEWMGIINPSGVSTTYAQKFQGRVTMTRDTSTSTVYMELSSLRSEDTAVYYCAEVRGSGFNYFGMDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 135 - protein, 122 aa, synthetic construct
EVQLVQSGAEVKKPGASVKVSCKASGDTFKRYYVHWVRQAPGQGLEWMGIINPSGVSTTYAQKFQGRVTMTRDTSTSTVYMELSSLRSEDTAVYYCAEVRGSGFNYFGMDVWGQGTLVTVSS

SEQ ID NO: 136 - protein, 213 aa, synthetic construct
SYELTQPPSVSVSPGQTASITCSGDKLGDKYASWYQQKPGQSPVLVIYQDNRRPSGIPERFSGSNSGNTATLTISGTQAMDEADYYCQAWDSDTYVFGTGTKVTVLRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 137 - protein, 106 aa, synthetic construct
SYELTQPPSVSVSPGQTASITCSGDKLGDKYASWYQQKPGQSPVLVIYQDNRRPSGIPERFSGSNSGNTATLTISGTQAMDEADYYCQAWDSDTYVFGTGTKVTVL

SEQ ID NO: 138 - protein, 446 aa, synthetic construct
QVQLVQSGPEVKKPGASVKVSCKASGYTFTNYGMNWVRQAPGQGLEWMGWINTYTGEPTYGEDFKGRFAFSLDTSASTAYMELSSLRSEDTAVYFCARFGNYVDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 139 - protein, 116 aa, synthetic construct
QVQLVQSGPEVKKPGASVKVSCKASGYTFTNYGMNWVRQAPGQGLEWMGWINTYTGEPTYGEDFKGRFAFSLDTSASTAYMELSSLRSEDTAVYFCARFGNYVDYWGQGSLVTVSS

SEQ ID NO: 140 - protein, 219 aa, synthetic construct
DIVMTQSPLSLPVTPGEPASISCRSSKNLLHSNGITYLYWYLQKPGQSPQLLIYQMSNLASGVPDRFSSSGSGTDFTLKISRVEAEDVGVYYCAQNLEIPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 141 - protein, 112 aa, synthetic construct
DIVMTQSPLSLPVTPGEPASISCRSSKNLLHSNGITYLYWYLQKPGQSPQLLIYQMSNLASGVPDRFSSSGSGTDFTLKISRVEAEDVGVYYCAQNLEIPRTFGQGTKVEIK

SEQ ID NO: 142 - protein, 446 aa, synthetic construct
QIQLVQSGPELKKPGETVKISCKASGYTFTKYGMNWVKQAPGKGLKWMGWINTYTEEPTYGDDFKGRFAFSLETSASTANLQINNLKSEDTATYFCARFGSAVDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 143 - protein, 116 aa, synthetic construct
QIQLVQSGPELKKPGETVKISCKASGYTFTKYGMNWVKQAPGKGLKWMGWINTYTEEPTYGDDFKGRFAFSLETSASTANLQINNLKSEDTATYFCARFGSAVDYWGQGTSVTVSS

SEQ ID NO: 144 - protein, 219 aa, synthetic construct
DIVMTQAAFSNPVTLGTSGSISCRSSKSLLHSNGITYLYWYLQKPGQSPQLLIYQMSNLASGVPDRFSSSGSGTDFTLRISRVEAEDVGVYYCAQNLELPRTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 145 - protein, 112 aa, synthetic construct
DIVMTQAAFSNPVTLGTSGSISCRSSKSLLHSNGITYLYWYLQKPGQSPQLLIYQMSNLASGVPDRFSSSGSGTDFTLRISRVEAEDVGVYYCAQNLELPRTFGGGTKLEIK

SEQ ID NO: 146 - protein, 446 aa, synthetic construct
EVQLVQSGPGLVQPGGSVRISCAASGYTFTNYGMNWVKQAPGKGLEWMGWINTYTGESTYADSFKGRFTFSLDTSASAAYLQINSLRAEDTAVYYCARFAIKGDYWGQGTLLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 147 - protein, 116 aa, synthetic construct
EVQLVQSGPGLVQPGGSVRISCAASGYTFTNYGMNWVKQAPGKGLEWMGWINTYTGESTYADSFKGRFTFSLDTSASAAYLQINSLRAEDTAVYYCARFAIKGDYWGQGTLLTVSS

SEQ ID NO: 148 - protein, 219 aa, synthetic construct
DIQMTQSPSSLSASVGDRVTITCRSTKSLLHSNGITYLYWYQQKPGKAPKLLIYQMSNLASGVPSRFSSSGSGTDFTLTISSLQPEDFATYYCAQNLEIPRTFGQGTKVELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 149 - protein, 112 aa, synthetic construct
DIQMTQSPSSLSASVGDRVTITCRSTKSLLHSNGITYLYWYQQKPGKAPKLLIYQMSNLASGVPSRFSSSGSGTDFTLTISSLQPEDFATYYCAQNLEIPRTFGQGTKVELK

SEQ ID NO: 150 - protein, 442 aa, synthetic construct
QVQLVQSGAEVKKPGASVKVSCKASGGTFSSYAISWVRQAPGQGLEWMGGIVPIFGTANYAQKFQGRVTITADESTSTAYMELSSLRSEDTAVYYCARDPFLHYWGQGTLVTASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 151 - protein, 112 aa, synthetic construct
QVQLVQSGAEVKKPGASVKVSCKASGGTFSSYAISWVRQAPGQGLEWMGGIVPIFGTANYAQKFQGRVTITADESTSTAYMELSSLRSEDTAVYYCARDPFLHYWGQGTLVT

SEQ ID NO: 152 - protein, 214 aa, synthetic construct
EIELTQSPGTLSLSPGERATLSCRASQSVSSSYLAWYQQKPGQAPRLLIYGASSRATGIPDRFSGSGSGTDFTLTISRLEPEDFAVYYCAQGELYPRQFGGGTKLDIRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 153 - protein, 107 aa, synthetic construct
EIELTQSPGTLSLSPGERATLSCRASQSVSSSYLAWYQQKPGQAPRLLIYGASSRATGIPDRFSGSGSGTDFTLTISRLEPEDFAVYYCAQGELYPRQFGGGTKLDI

SEQ ID NO: 154 - protein, 445 aa, synthetic construct
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCARGLLWNYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 155 - protein, 115 aa, synthetic construct
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCARGLLWNYWGQGTLVTVSS

SEQ ID NO: 156 - protein, 216 aa, synthetic construct
EIVMTQSPATLSVSPGERATLSCRASQSVSSNLAWYQQKPGQAPRLIIYGASTTASGIPARFSASGSGTDFTLTISSLQSEDFAVYYCQQYNNWPPAYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 157 - protein, 109 aa, synthetic construct
EIVMTQSPATLSVSPGERATLSCRASQSVSSNLAWYQQKPGQAPRLIIYGASTTASGIPARFSASGSGTDFTLTISSLQSEDFAVYYCQQYNNWPPAYTFGQGTKLEIK

SEQ ID NO: 158 - protein, 449 aa, synthetic construct
EVQLVESGGGLVQPGGSLRLSCAASGFTFSSYVMSWVRQAPGKGLEWVATISSGGSYTYYPDSVKGRFTISRDNAKNTLYLQMNSLRAEDTAVYYCARRGDSMITTDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 159 - protein, 119 aa, synthetic construct [sequence omitted]

SEQ ID NO: 160 - protein, 213 aa, synthetic construct
DIQMTQSPSSLSASVGDRVTITCKASQDVGTAVAWYQQKPGKAPKLLIYWASTRHTGVPSRFSGSGSGTDFTLTISSLQPEDFATYYCQQYSSYRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 161 - protein, 106 aa, synthetic construct [sequence omitted]

SEQ ID NO: 162 - protein, 451 aa, synthetic construct
EVQLVQSGGGVERPGGSLRLSCAASGFTFDDYAMSWVRQAPGKGLEWVSGINWQGGSTGYADSVKGRVTISRDNAKNSLYLQMNSLRAEDTAVYYCAKILGAGRGWYFDYWGKGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 163 - protein, 121 aa, synthetic construct [sequence omitted]

SEQ ID NO: 164 - protein, 214 aa, synthetic construct
SELTQDPAVSVALGQTVRITCSGDSLRSYYASWYQQKPGQAPVLVIYGANNRPSGIPDRFSGSSSGNTASLTITGAQAEDEADYYCNSADSSGNHVVFGGGTKLTVLRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 165 - protein, 107 aa, synthetic construct [sequence omitted]

SEQ ID NO: 166 - protein, 448 aa, synthetic construct
EVQLQQSGAEVVKPGASVKLSCKASGFNIKDTFIHWVKQAPGQGLEWIGRIDPANTNTKYDPKFQGKATITTDTSSNTAYMELSSLRSEDTAVYYCVRGLYTYYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 167 - protein, 118 aa, synthetic construct
EVQLQQSGAEVVKPGASVKLSCKASGFNIKDTFIHWVKQAPGQGLEWIGRIDPANTNTKYDPKFQGKATITTDTSSNTAYMELSSLRSEDTAVYYCVRGLYTYYFDYWGQGTLVTVSS

SEQ ID NO: 168 - protein, 214 aa, synthetic construct
EIVMTQSPATLSVSPGERATLSCRASQSISNNLHWYQQKPGQAPRLLIKFASQSITGIPARFSGSGSGTEFTLTISSLQSEDFAVYYCQQGNSWPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 169 - protein, 107 aa, synthetic construct
EIVMTQSPATLSVSPGERATLSCRASQSISNNLHWYQQKPGQAPRLLIKFASQSITGIPARFSGSGSGTEFTLTISSLQSEDFAVYYCQQGNSWPYTFGQGTKLEIK

SEQ ID NO: 170 - protein, 452 aa, synthetic construct
QVQLQESGPGLVKPSQTLSLTCTVSGGSISSGDYFWSWIRQLPGKGLEWIGHIHNSGTTYYNPSLKSRVTISVDTSKKQFSLRLSSVTAADTAVYYCARDRGGDYYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 171 - protein, 122 aa, synthetic construct
QVQLQESGPGLVKPSQTLSLTCTVSGGSISSGDYFWSWIRQLPGKGLEWIGHIHNSGTTYYNPSLKSRVTISVDTSKKQFSLRLSSVTAADTAVYYCARDRGGDYYYGMDVWGQGTTVTVSS

SEQ ID NO: 172 - protein, 215 aa, synthetic construct
EIVLTQSPGTLSLSPGERATLSCRASQGISRSYLAWYQQKPGQAPSLLIYGASSRATGIPDRFSGSGSGTDFTLTISRLEPEDFAVYYCQQFGSSPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 173 - protein, 108 aa, synthetic construct
EIVLTQSPGTLSLSPGERATLSCRASQGISRSYLAWYQQKPGQAPSLLIYGASSRATGIPDRFSGSGSGTDFTLTISRLEPEDFAVYYCQQFGSSPWTFGQGTKVEIK

SEQ ID NO: 174 - protein, 451 aa, synthetic construct
EVQLVQSGGGVERPGGSLRLSCAASGFTFDDYGMSWVRQAPGKGLEWVSGINWNGGSTGYADSVKGRVTISRDNAKNSLYLQMNSLRAEDTAVYYCAKILGAGRGWYFDLWGKGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 175 - protein, 121 aa, synthetic construct
EVQLVQSGGGVERPGGSLRLSCAASGFTFDDYGMSWVRQAPGKGLEWVSGINWNGGSTGYADSVKGRVTISRDNAKNSLYLQMNSLRAEDTAVYYCAKILGAGRGWYFDLWGKGTTVTVSS

SEQ ID NO: 176 - protein, 215 aa, synthetic construct
SSELTQDPAVSVALGQTVRITCQGDSLRSYYASWYQQKPGQAPVLVIYGKNNRPSGIPDRFSGSSSGNTASLTITGAQAEDEADYYCNSRDSSGNHVVFGGGTKLTVLRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 177 - protein, 108 aa, synthetic construct
SSELTQDPAVSVALGQTVRITCQGDSLRSYYASWYQQKPGQAPVLVIYGKNNRPSGIPDRFSGSSSGNTASLTITGAQAEDEADYYCNSRDSSGNHVVFGGGTKLTVL

SEQ ID NO: 178 - protein, 454 aa, synthetic construct
EVQLVESGGGLVQPGGSLRLSCAVSGYSITSDYAWNWVRQAPGKGLEWVGYISNSGSTSYNPSLKSRFTISRDTSKNTLYLQMNSLRAEDTAVYYCARERNYDYDDYYYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 179 - protein, 124 aa, synthetic construct
EVQLVESGGGLVQPGGSLRLSCAVSGYSITSDYAWNWVRQAPGKGLEWVGYISNSGSTSYNPSLKSRFTISRDTSKNTLYLQMNSLRAEDTAVYYCARERNYDYDDYYYAMDYWGQGTLVTVSS

SEQ ID NO: 180 - protein, 220 aa, synthetic construct
DIQMTQSPSSLSASVGDRVTITCKSSQSLLYRSNQKNYLAWYQQKPGKAPKLLIYWASTRESGVPSRFSGSGSGTDFTLTISSLQPEDFATYYCQQYYNYPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 181 - protein, 113 aa, synthetic construct
DIQMTQSPSSLSASVGDRVTITCKSSQSLLYRSNQKNYLAWYQQKPGKAPKLLIYWASTRESGVPSRFSGSGSGTDFTLTISSLQPEDFATYYCQQYYNYPRTFGQGTKVEIK

SEQ ID NO: 182 - protein, 450 aa, synthetic construct
EVQLVESGGGLVQPGGSLRLSCAASGFTFSRYVMSWVRQAPGKGLEWVASISSGGRMYYPDTVKGRFTISRDNAKNSLYLQMNSLRAEDTAVYYCARGSIYDGYYVFPYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 183 - protein, 120 aa, synthetic construct
EVQLVESGGGLVQPGGSLRLSCAASGFTFSRYVMSWVRQAPGKGLEWVASISSGGRMYYPDTVKGRFTISRDNAKNSLYLQMNSLRAEDTAVYYCARGSIYDGYYVFPYWGQGTLVTVSS

SEQ ID NO: 184 - protein, 215 aa, synthetic construct
EIVLTQSPATLSLSPGERATLSCSASSSVSSSYLYWYQQKPGQAPRLLIYSTSNLASGIPARFSGSGSGTDFTLTISSLEPEDFAVYYCHQWSTYPPTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 185 - protein, 108 aa, synthetic construct
EIVLTQSPATLSLSPGERATLSCSASSSVSSSYLYWYQQKPGQAPRLLIYSTSNLASGIPARFSGSGSGTDFTLTISSLEPEDFAVYYCHQWSTYPPTFGGGTKVEIK

SEQ ID NO: 186 - protein, 455 aa, synthetic construct
QVQLQESGPGLVKPSQTLSLTCTVSGGSISSGGYYWSWIRQHPGKGLEWIGYIYYSGRTYNNPSLKSRVTISVDTSKNQFSLKLSSVTAADTAVYYCARVATGRADYHFYAMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 187 - protein, 125 aa, synthetic construct
QVQLQESGPGLVKPSQTLSLTCTVSGGSISSGGYYWSWIRQHPGKGLEWIGYIYYSGRTYNNPSLKSRVTISVDTSKNQFSLKLSSVTAADTAVYYCARVATGRADYHFYAMDVWGQGTTVTVSS

SEQ ID NO: 188 - protein, 213 aa, synthetic construct
SYELTQPSSVSVSPGQTARITCSGDVLAKKSARWFHQKPGQAPVLVIYKDSERPSGIPERFSGSSSGTTVTLTISGAQVEDEAAYYCYSAADNNLVFGGGTKLTVLRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 189 - protein, 106 aa, synthetic construct
SYELTQPSSVSVSPGQTARITCSGDVLAKKSARWFHQKPGQAPVLVIYKDSERPSGIPERFSGSSSGTTVTLTISGAQVEDEAAYYCYSAADNNLVFGGGTKLTVL

SEQ ID NO: 190 - protein, 449 aa, synthetic construct
QVQLVQSGAEVKKPGASVKVSCKASGYTFTNYRIEWVRQAPGQGLEWMGEILPRGGNTNYNEKFKGRVTFTADTSTSTAYMELRSLRSDDTAVYYCARDDGYYGRFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 191 - protein, 119 aa, synthetic construct
QVQLVQSGAEVKKPGASVKVSCKASGYTFTNYRIEWVRQAPGQGLEWMGEILPRGGNTNYNEKFKGRVTFTADTSTSTAYMELRSLRSDDTAVYYCARDDGYYGRFAYWGQGTLVTVSS

SEQ ID NO: 192 - protein, 214 aa, synthetic construct
DIQMTQSPSSLSASVGDRVTITCRASQDISNYLNWYQQKPGKAPKLLIYYTSRLHSGVPSRFSGSGSGTDYTLTISSLQPEDFATYYCQQGNTLPPTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 193 - protein, 107 aa, synthetic construct
DIQMTQSPSSLSASVGDRVTITCRASQDISNYLNWYQQKPGKAPKLLIYYTSRLHSGVPSRFSGSGSGTDYTLTISSLQPEDFATYYCQQGNTLPPTFGGGTKVEIK

SEQ ID NO: 194 - protein, 449 aa, synthetic construct
QVQLQESGPGLVKPSETLSLTCTVSGDSITSGYWNWIRQPPGKGLEYIGYISDSGITYYNPSLKSRVTISRDTSKNQYSLKLSSVTAADTAVYYCARRTLATYYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 195 - protein, 119 aa, synthetic construct
QVQLQESGPGLVKPSETLSLTCTVSGDSITSGYWNWIRQPPGKGLEYIGYISDSGITYYNPSLKSRVTISRDTSKNQYSLKLSSVTAADTAVYYCARRTLATYYAMDYWGQGTLVTVSS

SEQ ID NO: 196 - protein, 219 aa, synthetic construct
DFVMTQSPLSLPVTLGQPASISCRASQSLVHSDGNTYLHWYQQRPGQSPRLLIYRVSNRFSGVPDRFSGSGSGTDFTLKISRVEAEDVGVYYCSQSTHVPPTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 197 - protein, 112 aa, synthetic construct
DFVMTQSPLSLPVTLGQPASISCRASQSLVHSDGNTYLHWYQQRPGQSPRLLIYRVSNRFSGVPDRFSGSGSGTDFTLKISRVEAEDVGVYYCSQSTHVPPTFGQGTKLEIK

SEQ ID NO: 198 - protein, 448 aa, synthetic construct
QVQLVQSGAEVKKPGASVKVSCKASGFNIKDTYIHWVRQAPGQGLEWMGRIDPANGKANYDPKFQGRVTMTRDTSTSTVYMELSSLRSEDTAVYYCALGGGYYGMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 199 - protein, 118 aa, synthetic construct
QVQLVQSGAEVKKPGASVKVSCKASGFNIKDTYIHWVRQAPGQGLEWMGRIDPANGKANYDPKFQGRVTMTRDTSTSTVYMELSSLRSEDTAVYYCALGGGYYGMDYWGQGTLVTVSS

SEQ ID NO: 200 - protein, 218 aa, synthetic construct
DIVMTQSPDSLAVSLGERATINCRASESVDSYGNSFMHWYQQKPGQPPKLLIYLASNLESGVPDRFSGSGSGTDFTLTISSLQAEDVAVYYCQQNNEDPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 201 - protein, 111 aa, synthetic construct
DIVMTQSPDSLAVSLGERATINCRASESVDSYGNSFMHWYQQKPGQPPKLLIYLASNLESGVPDRFSGSGSGTDFTLTISSLQAEDVAVYYCQQNNEDPLTFGQGTKVEIK

SEQ ID NO: 202 - protein, 454 aa, synthetic construct
QQQLEEYGGDLVQPEGSLTLTCKASGLDFSSSYWMCWVRQAPGKGLEWIACIYTGSSGSTSYASWAKGRFTISKTSSTTVTLQMTSLTAADTATYFCARDYDYTAYAYGIMSLWGPGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 203 - protein, 124 aa, synthetic construct
QQQLEEYGGDLVQPEGSLTLTCKASGLDFSSSYWMCWVRQAPGKGLEWIACIYTGSSGSTSYASWAKGRFTISKTSSTTVTLQMTSLTAADTATYFCARDYDYTAYAYGIMSLWGPGTLVTVSS

SEQ ID NO: 204 - protein, 220 aa, synthetic construct
AEIVMTQTPSSKSAAVGDTVTIKCQASQSITSYLSWYQQKPGQPPKLLIYRASTLASGVPSRFKGSGSGTQFTLTISDLECADAATYYCQSNYGSYSTNYGVTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 205 - protein, 113 aa, synthetic construct
AEIVMTQTPSSKSAAVGDTVTIKCQASQSITSYLSWYQQKPGQPPKLLIYRASTLASGVPSRFKGSGSGTQFTLTISDLECADAATYYCQSNYGSYSTNYGVTFGGGTKVEIK

SEQ ID NO: 206 - protein, 446 aa, synthetic construct
QEQLVESGGGLVQPEGSLTLTCTASGFSFSSRYYMCWVRQAPGKGLEWIGCIYTGSGSTYYASWAKGRVTISKTSSTTVTLQMTSLTAADTATYFCAREAGSFNLWGPGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 207 - protein, 116 aa, synthetic construct
QEQLVESGGGLVQPEGSLTLTCTASGFSFSSRYYMCWVRQAPGKGLEWIGCIYTGSGSTYYASWAKGRVTISKTSSTTVTLQMTSLTAADTATYFCAREAGSFNLWGPGTLVTVSS

SEQ ID NO: 208 - protein, 217 aa, synthetic construct
DVVMTQTPASVSEPVGGTVTIKCQASQSIYSGLAWYQQKPGQPPKLLIYSASKLASGVPSRFKGSGSGTEYTLTISDLECADAATYYCQNYYYGISNGWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 209 - protein, 110 aa, synthetic construct
DVVMTQTPASVSEPVGGTVTIKCQASQSIYSGLAWYQQKPGQPPKLLIYSASKLASGVPSRFKGSGSGTEYTLTISDLECADAATYYCQNYYYGISNGWTFGGGTKVEIK

SEQ ID NO: 210 - protein, 359 aa, synthetic construct
QVQLQESGGGLVQAGGSLRLSCAASGSIFWKPVMGWYRQAPGKEREFVAAITSGTNTYYADSVKGRFTISRDNAKNTVYLQMNSLKPEDTAVYYCAVDDYDVVEYPYWGQGTQVTVSSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGSHHHHHH

SEQ ID NO: 211 - protein, 118 aa, synthetic construct
QVQLQESGGGLVQAGGSLRLSCAASGSIFWKPVMGWYRQAPGKEREFVAAITSGTNTYYADSVKGRFTISRDNAKNTVYLQMNSLKPEDTAVYYCAVDDYDVVEYPYWGQGTQVTVSS

SEQ ID NO: 212 - protein, 359 aa, synthetic construct
QVQLQESGGGLVQAGGSLRLSCAASGNISVQLDMGWYRQAPGKEREFVAAINQGTTTYYADSVKGRFTISRDNAKNTVYLQMNSLKPEDTAVYYCAVYLYDIWNHPYWGQGTQVTVSSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGSHHHHHH

SEQ ID NO: 213 - protein, 118 aa, synthetic construct
QVQLQESGGGLVQAGGSLRLSCAASGNISVQLDMGWYRQAPGKEREFVAAINQGTTTYYADSVKGRFTISRDNAKNTVYLQMNSLKPEDTAVYYCAVYLYDIWNHPYWGQGTQVTVSS

SEQ ID NO: 214 - protein, 359 aa, synthetic construct
QVQLQESGGGLVQAGGSLRLSCAASGSISGGKGMGWYRQAPGKEREFVAAIGSGAITYYADSVKGRFTISRDNAKNTVYLQMNSLKPEDTAVYYCAVYTTALDEYPYWGQGTQVTVSSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGSHHHHHH

SEQ ID NO: 215 - protein, 118 aa, synthetic construct
QVQLQESGGGLVQAGGSLRLSCAASGSISGGKGMGWYRQAPGKEREFVAAIGSGAITYYADSVKGRFTISRDNAKNTVYLQMNSLKPEDTAVYYCAVYTTALDEYPYWGQGTQVTVSS

SEQ ID NO: 216 - protein, 367 aa, synthetic construct
QVQLQESGGGLVQAGGSLRLSCAASGNISYFLIMGWYRQAPGKEREFVAAITRGSNTYYADSVKGRFTISRDNAKNTVYLQMNSLKPEDTAVYYCAVFSTLQYHYDTGYTAYLTYWGQGTQVTVSSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGSHHHHHH

SEQ ID NO: 217 - protein, 126 aa, synthetic construct
QVQLQESGGGLVQAGGSLRLSCAASGNISYFLIMGWYRQAPGKEREFVAAITRGSNTYYADSVKGRFTISRDNAKNTVYLQMNSLKPEDTAVYYCAVFSTLQYHYDTGYTAYLTYWGQGTQVTVSS

SEQ ID NO: 218 - protein, 450 aa, synthetic construct
QVQLQQSGPDLVKPGASVRISCKASGYTFAGHYVHWVKQRPGRGLEWIGWIFPGKVNTKYNEKFKGKATLTADKSSSTAYMQLSSLTSEDSAVYFCARVGYDYPYYFDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 219 - protein, 120 aa, synthetic construct
QVQLQQSGPDLVKPGASVRISCKASGYTFAGHYVHWVKQRPGRGLEWIGWIFPGKVNTKYNEKFKGKATLTADKSSSTAYMQLSSLTSEDSAVYFCARVGYDYPYYFDYWGQGTTLTVSS

SEQ ID NO: 220 - protein, 214 aa, synthetic construct
DILLTQSPAILSVSPGDRVSFSCRASQSIGTSIHWYQQRTDGSPRLLIKYASESISGIPSRFSGSGSGTDFTLSINSVESEDVADYYCQQSSSWPFTFGSGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 221 - protein, 107 aa, synthetic construct
DILLTQSPAILSVSPGDRVSFSCRASQSIGTSIHWYQQRTDGSPRLLIKYASESISGIPSRFSGSGSGTDFTLSINSVESEDVADYYCQQSSSWPFTFGSGTKLEIK

SEQ ID NO: 222 - protein, 449 aa, synthetic construct
EVQLLESGGGLVQPGGSLRLSCAASGFTFSHYVMAWVRQAPGKGLEWVSSISSSGGWTLYADSVKGRFTISRDNSKNTLYLQMNSLRAEDTAVYYCTRGLKMATIFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 223 - protein, 119 aa, synthetic construct [sequence omitted]

SEQ ID NO: 224 - protein, 217 aa, synthetic construct
QSALTQPASVSGSPGQSITISCTGTSSDVGSYNVVSWYQQHPGKAPKLIIYEVSQRPSGVSNRFSGSKSGNTASLTISGLQTEDEADYYCCSYAGSSIFVIFGGGTKVTVLGQPKAAPSVTLFPPSSEELQANKATLVCLVSDFYPGAVTVAWKADGSPVKVGVETTKPSKQSNNKYAASSYLSLTPEQWKSHRSYSCRVTHEGSTVEKTVAPAECS

SEQ ID NO: 225 - protein, 111 aa, synthetic construct [sequence omitted]

SEQ ID NO: 226 - protein, 452 aa, synthetic construct
QVQLQESGPELKKPGETVKISCKASGYTFRNYGMNWVKQAPGKGLKWMGWINTYTGEPTYADDFKGRFAFSLETSASTAYLQINNVKNEDTATYFCARKGWMDFNGSSLDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 227 - protein, 122 aa, synthetic construct
QVQLQESGPELKKPGETVKISCKASGYTFRNYGMNWVKQAPGKGLKWMGWINTYTGEPTYADDFKGRFAFSLETSASTAYLQINNVKNEDTATYFCARKGWMDFNGSSLDYWGQGTTVTVSS

SEQ ID NO: 228 - protein, 219 aa, synthetic construct
SIVMTQTPLSLPVSLGDQASISCQSSQSIVHSNGNTYLEWYLQKPGQSPNLLIYKVSNRFSGVPDRFSGSGSGTDFTLKISRVEAEDIGVYYCFQGSHVPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 229 - protein, 112 aa, synthetic construct
SIVMTQTPLSLPVSLGDQASISCQSSQSIVHSNGNTYLEWYLQKPGQSPNLLIYKVSNRFSGVPDRFSGSGSGTDFTLKISRVEAEDIGVYYCFQGSHVPPTFGGGTKLEIK

SEQ ID NO: 230 - protein, 451 aa, synthetic construct
EVRLVESGGGLVQGPGSLRLSCAASGFALTDYYMSWVRQSPGKTLEWLGFIANKANGHTTDYSPSVKGRFTISRDNSQTILYLQMNTLRTEDSATYYCARDMGIRWNFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 231 - protein, 121 aa, synthetic construct
EVRLVESGGGLVQGPGSLRLSCAASGFALTDYYMSWVRQSPGKTLEWLGFIANKANGHTTDYSPSVKGRFTISRDNSQTILYLQMNTLRTEDSATYYCARDMGIRWNFDVWGQGTTVTVSS

SEQ ID NO: 232 - protein, 213 aa, synthetic construct
DIQLTQSPAIMSASPGEKVTMTCSASSRVSYIHWYQQKSGTSPKRWIYGTSTLASGVPARFSGSGSGTSYSLTISSMEAEDAATYYCQQWSYNPPTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 233 - protein, 106 aa, synthetic construct
DIQLTQSPAIMSASPGEKVTMTCSASSRVSYIHWYQQKSGTSPKRWIYGTSTLASGVPARFSGSGSGTSYSLTISSMEAEDAATYYCQQWSYNPPTFGAGTKLELK

SEQ ID NO: 234 - protein, 449 aa, synthetic construct
EVQLQESGPGLVKPSQTLSLTCTVSDGSVSRGGYYLTWIRQHPGKGLEWIGYIYYSGSTYFNPSLRSRVTMSVDTSKNQFSLKLSSVTAADTAVYYCARGIAVAPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 235 - protein, 119 aa, synthetic construct
EVQLQESGPGLVKPSQTLSLTCTVSDGSVSRGGYYLTWIRQHPGKGLEWIGYIYYSGSTYFNPSLRSRVTMSVDTSKNQFSLKLSSVTAADTAVYYCARGIAVAPFDYWGQGTLVTVSS

SEQ ID NO: 236 - protein, 214 aa, synthetic construct
EIVLTQSPATLSVSPGERATLSCRTSQSVRSNLAWYQQKPGQAPRLLIYAASTRATGIPARFSGSGSGTEFTLTISSLQSEDFAVYYCQQYTNWPFTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 237 - protein, 107 aa, synthetic construct
EIVLTQSPATLSVSPGERATLSCRTSQSVRSNLAWYQQKPGQAPRLLIYAASTRATGIPARFSGSGSGTEFTLTISSLQSEDFAVYYCQQYTNWPFTFGPGTKVDIK

SEQ ID NO: 238 - protein, 450 aa, synthetic construct
QMQLVQSEAELKKPGASVKVSCKASGYSFTSHFMHWVRQAPGQGLEWMGWIDPVTGGTKYAQNFQGWVTMTRDTSIRTAYLELSRLRSDDTAMYYCAREARADRGQFDKWGQGTLVTVASASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 239 - protein, 120 aa, synthetic construct
QMQLVQSEAELKKPGASVKVSCKASGYSFTSHFMHWVRQAPGQGLEWMGWIDPVTGGTKYAQNFQGWVTMTRDTSIRTAYLELSRLRSDDTAMYYCAREARADRGQFDKWGQGTLVTVAS

SEQ ID NO: 240 - protein, 214 aa, synthetic construct
QSVLTQPPSVSVAPGKTARITCGGNNIGSKSVHWYQQKPGQAPALVIYYGSNRPSGIPERFSGSNSGNTATLTISRVEAGDEADYYCQVWDSSSDWVFGGGTKLTVLRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 241 - protein, 107 aa, synthetic construct
QSVLTQPPSVSVAPGKTARITCGGNNIGSKSVHWYQQKPGQAPALVIYYGSNRPSGIPERFSGSNSGNTATLTISRVEAGDEADYYCQVWDSSSDWVFGGGTKLTVL

SEQ ID NO: 242 - protein, 446 aa, synthetic construct
EVQLVQSGAEVKKPGASVKVSCKASGYTFTSYWMHWVRQAPGQRLEWIGEINPTNGRTNYIEKFKSRATLTVDKSASTAYMELSSLRSEDTAVYYCARGTRAYHYWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 243 - protein, 116 aa, synthetic construct
EVQLVQSGAEVKKPGASVKVSCKASGYTFTSYWMHWVRQAPGQRLEWIGEINPTNGRTNYIEKFKSRATLTVDKSASTAYMELSSLRSEDTAVYYCARGTRAYHYWGQGTMVTVSS

SEQ ID NO: 244 - protein, 214 aa, synthetic construct
DIQMTQSPSSLSASVGDRVTITCRASDNLYSNLAWYQQKPGKSPKLLVYDATNLADGVPSRFSGSGSGTDYTLTISSLQPEDFATYYCQHFWGTPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 245 - protein, 107 aa, synthetic construct
DIQMTQSPSSLSASVGDRVTITCRASDNLYSNLAWYQQKPGKSPKLLVYDATNLADGVPSRFSGSGSGTDYTLTISSLQPEDFATYYCQHFWGTPLTFGQGTKVEIK

SEQ ID NO: 246 - protein, 446 aa, synthetic construct
EVQLVQSGAEVKKPGASVKVSCKASGYTFTSYWMHWVRQAPGQRLEWIGEIAPTNGRTNYIEKFKSRATLTVDKSASTAYMELSSLRSEDTAVYYCARGTRAYHYWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 247 - protein, 116 aa, synthetic construct
EVQLVQSGAEVKKPGASVKVSCKASGYTFTSYWMHWVRQAPGQRLEWIGEIAPTNGRTNYIEKFKSRATLTVDKSASTAYMELSSLRSEDTAVYYCARGTRAYHYWGQGTMVTVSS

SEQ ID NO: 248 - protein, 214 aa, synthetic construct; identical to SEQ ID NO: 244

SEQ ID NO: 249 - protein, 107 aa, synthetic construct; identical to SEQ ID NO: 245

SEQ ID NO: 250 - protein, 446 aa, synthetic construct
EVQLVQSGAEVKKPGASVKVSCKASGYTFTSYWMHWVRQAPGQRLEWIGEINPANGRTNYIEKFKSRATLTVDKSASTAYMELSSLRSEDTAVYYCARGTRAYHYWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 251 - protein, 116 aa, synthetic construct
EVQLVQSGAEVKKPGASVKVSCKASGYTFTSYWMHWVRQAPGQRLEWIGEINPANGRTNYIEKFKSRATLTVDKSASTAYMELSSLRSEDTAVYYCARGTRAYHYWGQGTMVTVSS

SEQ ID NO: 252 - protein, 214 aa, synthetic construct; identical to SEQ ID NO: 244

SEQ ID NO: 253 - protein, 107 aa, synthetic construct; identical to SEQ ID NO: 245

SEQ ID NO: 254 - protein, 446 aa, synthetic construct; identical to SEQ ID NO: 250

SEQ ID NO: 255 - protein, 116 aa, synthetic construct; identical to SEQ ID NO: 251

SEQ ID NO: 256 - protein, 214 aa, synthetic construct
DIQMTQSPSSLSASVGDRVTITCRASDNLYSNLAWYQQKPGKSPKLLVYDATNLADGVPSRFSGSGSGTDYTLTISSLQPEDFATYYCQHFAGTPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 257 - protein, 107 aa, synthetic construct
DIQMTQSPSSLSASVGDRVTITCRASDNLYSNLAWYQQKPGKSPKLLVYDATNLADGVPSRFSGSGSGTDYTLTISSLQPEDFATYYCQHFAGTPLTFGQGTKVEIK

SEQ ID NO: 258 - protein, 461 aa, synthetic construct
EVQLVESGGGLVQPGGSLRLSCAASGFTFSSFGMHWVRQAPGKGLEWVAYISSDSSAIYYADTVKGRFTISRDNAKNSLYLQMNSLRDEDTAVYYCGRGRENIYYGSRLDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGSHHHHHH

SEQ ID NO: 259 - protein, 122 aa, synthetic construct
EVQLVESGGGLVQPGGSLRLSCAASGFTFSSFGMHWVRQAPGKGLEWVAYISSDSSAIYYADTVKGRFTISRDNAKNSLYLQMNSLRDEDTAVYYCGRGRENIYYGSRLDYWGQGTTVTVSS

SEQ ID NO: 260 - protein, 214 aa, synthetic construct
DIQLTQSPSFLSASVGDRVTITCKASQNVDTNVAWYQQKPGKAPKALIYSASYRYSGVPSRFSGSGSGTDFTLTISSLQPEDFATYYCQQYNNYPFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 261 - protein, 107 aa, synthetic construct
DIQLTQSPSFLSASVGDRVTITCKASQNVDTNVAWYQQKPGKAPKALIYSASYRYSGVPSRFSGSGSGTDFTLTISSLQPEDFATYYCQQYNNYPFTFGQGTKLEIK

SEQ ID NO: 262 - protein, 449 aa, synthetic construct
EVQLVESGGGVVQPGRSLRLSCSASGFDFTTYWMSWVRQAPGKGLEWIGEIHPDSSTINYAPSLKDRFTISRDNAKNTLFLQMDSLRPEDTGVYFCASLYFGFPWFAYWGQGTPVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 263 - protein, 119 aa, synthetic construct
EVQLVESGGGVVQPGRSLRLSCSASGFDFTTYWMSWVRQAPGKGLEWIGEIHPDSSTINYAPSLKDRFTISRDNAKNTLFLQMDSLRPEDTGVYFCASLYFGFPWFAYWGQGTPVTVSS

SEQ ID NO: 264 - protein, 213 aa, synthetic construct
DIQLTQSPSSLSASVGDRVTITCKASQDVGTSVAWYQQKPGKAPKLLIYWTSTRHTGVPSRFSGSGSGTDFTFTISSLQPEDIATYYCQQYSLYRSFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 265 - protein, 106 aa, synthetic construct
DIQLTQSPSSLSASVGDRVTITCKASQDVGTSVAWYQQKPGKAPKLLIYWTSTRHTGVPSRFSGSGSGTDFTFTISSLQPEDIATYYCQQYSLYRSFGQGTKVEIK

SEQ ID NO: 266 - protein, 457 aa, synthetic construct; identical to SEQ ID NO: 5

SEQ ID NO: 267 - protein, 127 aa, synthetic construct; identical to SEQ ID NO: 6

SEQ ID NO: 268 - protein, 214 aa, synthetic construct; identical to SEQ ID NO: 7

SEQ ID NO: 269 - protein, 107 aa, synthetic construct; identical to SEQ ID NO: 8

SEQ ID NO: 270 - protein, 450 aa, synthetic construct
EVQLQESGPGLVKPGGSLSLSCAASGFVFSSYDMSWVRQTPERGLEWVAYISSGGGITYAPSTVKGRFTVSRDNAKNTLYLQMNSLTSEDTAVYYCAAHYFGSSGPFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 271 - protein, 120 aa, synthetic construct [sequence omitted]

SEQ ID NO: 272 - protein, 214 aa, synthetic construct
DIQMTQSPASLSASVGDRVTITCRASENIFSYLAWYQQKPGKSPKLLVYNTRTLAEGVPSRFSGSGSGTDFSLTISSLQPEDFATYYCQHHYGTPFTFGSGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 273 - protein, 107 aa, synthetic construct [sequence omitted]

SEQ ID NO: 274 - protein, 458 aa, synthetic construct
QVQLKESGPGLVQPSQTLSLTCTVSGFSLTTYSVHWVRQHSGKNLEWMGRMWTAGDTSYNSAFTSRLNIFRDTSKSQVFLKMNSLQTEDTGTYYCARSSYTSGYPFDSWGQGVMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGSHHHHHH

SEQ ID NO: 275 - protein, 119 aa, synthetic construct
QVQLKESGPGLVQPSQTLSLTCTVSGFSLTTYSVHWVRQHSGKNLEWMGRMWTAGDTSYNSAFTSRLNIFRDTSKSQVFLKMNSLQTEDTGTYYCARSSYTSGYPFDSWGQGVMVTVSS

SEQ ID NO: 276 - protein, 212 aa, synthetic construct
DTVLTQSPALAVSPGERVTISCRASESVSKLMHWYQQRPGQQPQLLIYLTSHLASGVPARFSGSGSGTDFTLTIDPVEADDTATYYCQQSRNDPTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 277 - protein, 105 aa, synthetic construct
DTVLTQSPALAVSPGERVTISCRASESVSKLMHWYQQRPGQQPQLLIYLTSHLASGVPARFSGSGSGTDFTLTIDPVEADDTATYYCQQSRNDPTFGAGTKLELK

SEQ ID NO: 278 - protein, 461 aa, synthetic construct
EVQLVESGGGLVQPGGSLRLSCVVSGFTFSYYDMHWVRQVTGKGLEWVSAIGTAGATYYPGSVKGRFTISRENAKNSLYLQMNSLRAGDTAVYYCARDRGYSGYDAYYFDFWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGSHHHHHH

SEQ ID NO: 279 - protein, 122 aa, synthetic construct
EVQLVESGGGLVQPGGSLRLSCVVSGFTFSYYDMHWVRQVTGKGLEWVSAIGTAGATYYPGSVKGRFTISRENAKNSLYLQMNSLRAGDTAVYYCARDRGYSGYDAYYFDFWGQGTLVTVSS

SEQ ID NO: 280 - protein, 214 aa, synthetic construct; identical to SEQ ID NO: 7

SEQ ID NO: 281 - protein, 107 aa, synthetic construct; identical to SEQ ID NO: 8

SEQ ID NO: 282 - protein, 461 aa, synthetic construct
EVQLVQSGGGLVQPGGSLRLSCAASGFTFSYYDMHWVRQVTGKGLEWVSTIGATGDTYYSDSVKGRFTISRQNAKNSLYLQINSLRAGDTAVYYCVRDRGYIGYDSYYFDNWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGSHHHHHH

SEQ ID NO: 283 - protein, 122 aa, synthetic construct
EVQLVQSGGGLVQPGGSLRLSCAASGFTFSYYDMHWVRQVTGKGLEWVSTIGATGDTYYSDSVKGRFTISRQNAKNSLYLQINSLRAGDTAVYYCVRDRGYIGYDSYYFDNWGQGTLVTVSS

SEQ ID NO: 284 - protein, 214 aa, synthetic construct; identical to SEQ ID NO: 7

SEQ ID NO: 285 - protein, 107 aa, synthetic construct; identical to SEQ ID NO: 8

SEQ ID NO: 286 - protein, 456 aa, synthetic construct
QVQLVQSGAEVKKPGASVKVSCKASGYSFSGYFMNWVRQAPGQGLEWMGLINPYNGDSFYNQKFKGRVTMTRQTSTSTVYMELSSLRSEDTAVYYCVRGLRRDFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGSHHHHHH

SEQ ID NO: 287 - protein, 117 aa, synthetic construct
QVQLVQSGAEVKKPGASVKVSCKASGYSFSGYFMNWVRQAPGQGLEWMGLINPYNGDSFYNQKFKGRVTMTRQTSTSTVYMELSSLRSEDTAVYYCVRGLRRDFDYWGQGTLVTVSS

SEQ ID NO: 288 - protein, 219 aa, synthetic construct
DVVMTQSPLSLPVTLGQPASISCKSSQSLLDSDGKTYLNWLFQRPGQSPRRLIYLVSELDSGVPDRFSGSGSGTDFTLKISRVEAEDVGVYYCWQGTHFPRTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 289 - protein, 112 aa, synthetic construct
DVVMTQSPLSLPVTLGQPASISCKSSQSLLDSDGKTYLNWLFQRPGQSPRRLIYLVSELDSGVPDRFSGSGSGTDFTLKISRVEAEDVGVYYCWQGTHFPRTFGGGTKLEIK

SEQ ID NO: 290 - protein, 456 aa, synthetic construct
QVQLQQWGAGLLKPSETLSLTCAVYGGSFSGYYWSWIRQPPGKGLEWIGEINHSGSTNYNPSLKSRVTISVETSKNQFSLKLSSVTAADTAVYYCARDKWTWYFDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGSHHHHHH

SEQ ID NO: 291 - protein, 117 aa, synthetic construct [sequence omitted]

SEQ ID NO: 292 - protein, 220 aa, synthetic construct
DIEMTQSPDSLAVSLGERATINCRSSQSVLYSSSNRNYLAWYQQNPGQPPKLLIYWASTRESGVPDRFSGSGSGTDFTLTISSLQAEDVAVYYCQQYYSTPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 293 - protein, 113 aa, synthetic construct [sequence omitted]

SEQ ID NO: 294 - protein, 448 aa, synthetic construct
EVQLLESGGGLVQPGGSLRLSCAASGFTFSNYAMSWVRQAPGKGLEWVSSISGSGDYTYYTDSVKGRFTISRDNSKNTLYLQMNSLRAEDTAVYYCARSPWGYYLDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 295 - protein, 118 aa, synthetic construct
EVQLLESGGGLVQPGGSLRLSCAASGFTFSNYAMSWVRQAPGKGLEWVSSISGSGDYTYYTDSVKGRFTISRDNSKNTLYLQMNSLRAEDTAVYYCARSPWGYYLDSWGQGTLVTVSS

SEQ ID NO: 296 - protein, 214 aa, synthetic construct
DIQMTQSPPSLSASAGDRVTITCRASQGISSRLAWYQQKPEKAPKSLIYAASSLQSGVPSRFSGSGSGTDFTLTISSLQPEDFATYYCQQYNSYPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 297 - protein, 107 aa, synthetic construct
DIQMTQSPPSLSASAGDRVTITCRASQGISSRLAWYQQKPEKAPKSLIYAASSLQSGVPSRFSGSGSGTDFTLTISSLQPEDFATYYCQQYNSYPYTFGQGTKLEIK

SEQ ID NO: 298 - protein, 359 aa, synthetic construct
QVQLQESGGGLVQAGGSLRLSCAASGYISGYYVMGWYRQAPGKEREFVASISYGASTYYADSVKGRFTISRDNAKNTVYLQMNSLKPEDTAVYYCAVDFDSNYAHTYWGQGTQVTVSSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGSHHHHHH

SEQ ID NO: 299 - protein, 118 aa, synthetic construct
QVQLQESGGGLVQAGGSLRLSCAASGYISGYYVMGWYRQAPGKEREFVASISYGASTYYADSVKGRFTISRDNAKNTVYLQMNSLKPEDTAVYYCAVDFDSNYAHTYWGQGTQVTVSS

SEQ ID NO: 300 - protein, 359 aa, synthetic construct
QVQLQESGGGLVQAGGSLRLSCAASGTISFIGYMGWYRQAPGKERELVASIASGTSTYYADSVKGRFTISRDNAKNTVYLQMNSLKPEDTAVYYCAATQYIQDVHRYWGQGTQVTVSSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGSHHHHHH

SEQ ID NO: 301 - protein, 118 aa, synthetic construct
QVQLQESGGGLVQAGGSLRLSCAASGTISFIGYMGWYRQAPGKERELVASIASGTSTYYADSVKGRFTISRDNAKNTVYLQMNSLKPEDTAVYYCAATQYIQDVHRYWGQGTQVTVSS

SEQ ID NO: 302 - protein, 448 aa, synthetic construct
QVQLVQSGAEVKKPGASVKMSCKASGYTFTSYWMHWVRQAPGQGLEWIGAIYPGNSETGYAQKFQGRATLTADTSTSTAYMELSSLRSEDTAVYYCTRENWDPGFAFWGQGTLITVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 303 - protein, 118 aa, synthetic construct
QVQLVQSGAEVKKPGASVKMSCKASGYTFTSYWMHWVRQAPGQGLEWIGAIYPGNSETGYAQKFQGRATLTADTSTSTAYMELSSLRSEDTAVYYCTRENWDPGFAFWGQGTLITVSS

SEQ ID NO: 304 - protein, 213 aa, synthetic construct
DIQMTQSPSSLSASVGDRVTITCSASSSVYYMYWFQQKPGKAPKLWIYSTSNLASGVPSRFSGSGSGTDYTLTISSMQPEDFATYYCQQRRNYPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 305 - protein, 106 aa, synthetic construct
DIQMTQSPSSLSASVGDRVTITCSASSSVYYMYWFQQKPGKAPKLWIYSTSNLASGVPSRFSGSGSGTDYTLTISSMQPEDFATYYCQQRRNYPYTFGQGTKLEIK

SEQ ID NO: 306 - protein, 451 aa, synthetic construct
EVQLVESGGGLVQPGGSLRLSCAASGFTLSGDWIHWVRQAPGKGLEWVGEISAAGGYTDYADSVKGRFTISADTSKNTAYLQMNSLRAEDTAVYYCARESRVSFEAAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 307 - protein, 121 aa, synthetic construct [sequence omitted]

SEQ ID NO: 308 - protein, 214 aa, synthetic construct
DIQMTQSPSSLSASVGDRVTITCRASQNIATDVAWYQQKPGKAPKLLIYSASFLYSGVPSRFSGSGSGTDFTLTISSLQPEDFATYYCQQSEPEPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 309 - protein, 107 aa, synthetic construct [sequence omitted]

SEQ ID NO: 310 - protein, 446 aa, synthetic construct
QFQLVQSGAEVKKPGASVKVSCKASGYSFTDYNVNWVRQAPGQGLEWIGVINPKYGTTRYNQKFKGRATLTVDKSTSTAYMELSSLRSEDTAVYYCTRGLNAWDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 311 - protein, 116 aa, synthetic construct
QFQLVQSGAEVKKPGASVKVSCKASGYSFTDYNVNWVRQAPGQGLEWIGVINPKYGTTRYNQKFKGRATLTVDKSTSTAYMELSSLRSEDTAVYYCTRGLNAWDYWGQGTLVTVSS

SEQ ID NO: 312 - protein, 214 aa, synthetic construct
DIQMTQSPSSLSASVGDRVTITCGASENIYGALNWYQQKPGKAPKLLIYGATNLEDGVPSRFSGSGSGRDYTFTISSLQPEDIATYYCQNVLTTPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 313 - protein, 107 aa, synthetic construct
DIQMTQSPSSLSASVGDRVTITCGASENIYGALNWYQQKPGKAPKLLIYGATNLEDGVPSRFSGSGSGRDYTFTISSLQPEDIATYYCQNVLTTPYTFGQGTKLEIK

SEQ ID NO: 314 - protein, 446 aa, synthetic construct; identical to SEQ ID NO: 242

SEQ ID NO: 315 - protein, 116 aa, synthetic construct; identical to SEQ ID NO: 243

SEQ ID NO: 316 - protein, 214 aa, synthetic construct; identical to SEQ ID NO: 256

SEQ ID NO: 317 - protein, 107 aa, synthetic construct; identical to SEQ ID NO: 257

SEQ ID NO: 318 - protein, 446 aa, synthetic construct; identical to SEQ ID NO: 246

SEQ ID NO: 319 - protein, 116 aa, synthetic construct; identical to SEQ ID NO: 247

SEQ ID NO: 320 - protein, 214 aa, synthetic construct; identical to SEQ ID NO: 256

SEQ ID NO: 321 - protein, 107 aa, synthetic construct; identical to SEQ ID NO: 257

SEQ ID NO: 322 - protein, 444 aa, synthetic construct
EVQLQQSGPVLVKPGASVKISCKASGYSFTGYYMHWVRQSLVKRLEWIGRINPYTGVPSYKHNFKDKASLTVDKSSSTAYMELHSLTSEDSAVYYCARELGGYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 323 - protein, 114 aa, synthetic construct
EVQLQQSGPVLVKPGASVKISCKASGYSFTGYYMHWVRQSLVKRLEWIGRINPYTGVPSYKHNFKDKASLTVDKSSSTAYMELHSLTSEDSAVYYCARELGGYWGQGTTLTVSS

SEQ ID NO: 324 - protein, 216 aa, synthetic construct
QAVVTQESALTTSPGETVTLTCRSSTGAVTTGNYPNWVQEKPDHLFTGLIAGTNNRAPGVPARFSGSLIGDKAALTITGAQTEDEAIYFCALWFSSHWVFGGGTKLTVLRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 325 - protein, 109 aa, synthetic construct
QAVVTQESALTTSPGETVTLTCRSSTGAVTTGNYPNWVQEKPDHLFTGLIAGTNNRAPGVPARFSGSLIGDKAALTITGAQTEDEAIYFCALWFSSHWVFGGGTKLTVL

SEQ ID NO: 326 - protein, 447 aa, synthetic construct
EVQLVESGGGLVQPGGSLRLSCAASGFTFSDYFMSWVRQAPGKGLEWVATISNGGTYTYYPDSVKGRFTISRDNSKNTLYLQMNSLRAEDTAVYYCARFDGYIFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 327 - protein, 117 aa, synthetic construct
EVQLVESGGGLVQPGGSLRLSCAASGFTFSDYFMSWVRQAPGKGLEWVATISNGGTYTYYPDSVKGRFTISRDNSKNTLYLQMNSLRAEDTAVYYCARFDGYIFDYWGQGTLVTVSS

SEQ ID NO: 328 - protein, 214 aa, synthetic construct
DIQMTQSPSSLSASVGDRVTITCRASQDISNYLNWYQQKPGKAPKLLIYYTSRLHSGVPSRFSGSGSGTDFTLTISSLQPEDFATYYCQQGNTLPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 329 - protein, 107 aa, synthetic construct
DIQMTQSPSSLSASVGDRVTITCRASQDISNYLNWYQQKPGKAPKLLIYYTSRLHSGVPSRFSGSGSGTDFTLTISSLQPEDFATYYCQQGNTLPYTFGQGTKVEIK

SEQ ID NO: 330 - protein, 443 aa, synthetic construct
QVQLVQSGAEVKKPGASVKVSCKASGYTFTRYWIEWVRQAPGQRLEWMGEILPGSGSTNYNEKFKGRVTITADTSASTAYMELSSLRSEDTAVYYCERRGAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 331 - protein, 113 aa, synthetic construct
QVQLVQSGAEVKKPGASVKVSCKASGYTFTRYWIEWVRQAPGQRLEWMGEILPGSGSTNYNEKFKGRVTITADTSASTAYMELSSLRSEDTAVYYCERRGAYWGQGTLVTVSS

SEQ ID NO: 332 - protein, 214 aa, synthetic construct
DIQMTQSPSSLSASVGDRVTITCKASEDIYNRLAWYQQKPGKAPKLLISGATSLETGVPSRFSGSGSGTDYTLTISSLQPEDFATYYCQQQWSTPPTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 333 - protein, 107 aa, synthetic construct
DIQMTQSPSSLSASVGDRVTITCKASEDIYNRLAWYQQKPGKAPKLLISGATSLETGVPSRFSGSGSGTDYTLTISSLQPEDFATYYCQQQWSTPPTFGGGTKVEIK

SEQ ID NO: 334 - protein, 447 aa, synthetic construct
QVQLVQSGAEVKKPGASVKVSCKASGYSFSGYFMNWVRQAPGQGLEWMGLINPYNGDSFYNQKFKGRVTMTRQTSTSTVYMELSSLRSEDTAVYYCVRGLRRDFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 335 - protein, 117 aa, synthetic construct; identical to SEQ ID NO: 287

SEQ ID NO: 336 - protein, 219 aa, synthetic construct; identical to SEQ ID NO: 288

SEQ ID NO: 337 - protein, 112 aa, synthetic construct; identical to SEQ ID NO: 289

SEQ ID NO: 338 - protein, 451 aa, synthetic construct
EVQLVESGGGLVQPGGSLRLSCAASGFTFTGNWIHWVRQAPGKGLEWVGEISPSGGYTDYADSVKGRFTISADTSKNTAYLQMNSLRAEDTAVYYCARESRVSYEAAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 339 - protein, 121 aa, synthetic construct
EVQLVESGGGLVQPGGSLRLSCAASGFTFTGNWIHWVRQAPGKGLEWVGEISPSGGYTDYADSVKGRFTISADTSKNTAYLQMNSLRAEDTAVYYCARESRVSYEAAMDYWGQGTLVTVSS

SEQ ID NO: 340 - protein, 214 aa, synthetic construct
DIQMTQSPSSLSASVGDRVTITCRASQDVSTAVAWYQQKPGKAPKLLIYSASFLYSGVPSRFSGSGSGTDFTLTISSLQPEDFATYYCQQSYPTPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 341 - protein, 107 aa, synthetic construct
DIQMTQSPSSLSASVGDRVTITCRASQDVSTAVAWYQQKPGKAPKLLIYSASFLYSGVPSRFSGSGSGTDFTLTISSLQPEDFATYYCQQSYPTPYTFGQGTKVEIK

SEQ ID NO: 342 - protein, 451 aa, synthetic construct
QVQLQQSGAELVRPGTSVKVSCKASGYDFNNDLIEWVKQRPGQGLEWIAVINPGSGRTNYNEKFKGKATLTADKSSSTVYMQLSSLTSDDSAVYFCAMIYYGPHSYAMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 343 - protein, 121 aa, synthetic construct
QVQLQQSGAELVRPGTSVKVSCKASGYDFNNDLIEWVKQRPGQGLEWIAVINPGSGRTNYNEKFKGKATLTADKSSSTVYMQLSSLTSDDSAVYFCAMIYYGPHSYAMDYWGQGTSVTVSS

SEQ ID NO: 344 - protein, 214 aa, synthetic construct
DIVMTQSHKFMSTVVGDRVSITCKASLDVRTAVAWYQQKPGQSPKLLIYSASYRYTGVPDRFTGSGSGTDFTFNIRSVQAEDLAVYYCQQHYGIPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 345 - protein, 107 aa, synthetic construct
DIVMTQSHKFMSTVVGDRVSITCKASLDVRTAVAWYQQKPGQSPKLLIYSASYRYTGVPDRFTGSGSGTDFTFNIRSVQAEDLAVYYCQQHYGIPWTFGGGTKLEIK

SEQ ID NO: 346 - protein, 450 aa, synthetic construct
QVQLQQSGAELARPGTSVKVSCKASGYAFTNYLIEWVKQRPGQGLEWIGVISPGSGIINYNEKFKGKATLTADKSSSTAYMQLSSLTSDDSAVYFCAAIDYSGPYAVDDWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 347 - protein, 120 aa, synthetic construct
QVQLQQSGAELARPGTSVKVSCKASGYAFTNYLIEWVKQRPGQGLEWIGVISPGSGIINYNEKFKGKATLTADKSSSTAYMQLSSLTSDDSAVYFCAAIDYSGPYAVDDWGQGTSVTVSS

SEQ ID NO: 348 - protein, 214 aa, synthetic construct
DIVMTQSHKFMSTSVGDRVSVTCKASQAVNTAVAWYQQKPGQSPKLLIYSASYGYTGVPDRFTGSGSGTDFTLTISSVQAEDLAVYYCQHHYGVPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 349 - protein, 107 aa, synthetic construct
DIVMTQSHKFMSTSVGDRVSVTCKASQAVNTAVAWYQQKPGQSPKLLIYSASYGYTGVPDRFTGSGSGTDFTLTISSVQAEDLAVYYCQHHYGVPWTFGGGTKLEIK

SEQ ID NO: 350 - protein, 450 aa, synthetic construct
QVTLRESGPALVKPTQTLTLTCTFSGFSLSTSGMSVGWIRQPPGKALEWLADIWWDDKKDYNPSLKSRLTISKDTSKNQVVLKVTNMDPADTATYYCARSMITNWYFDVWGAGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 351 - protein, 120 aa, synthetic construct; identical to SEQ ID NO: 10

SEQ ID NO: 352 - protein, 213 aa, synthetic construct; identical to SEQ ID NO: 11

SEQ ID NO: 353 - protein, 106 aa, synthetic construct; identical to SEQ ID NO: 12

SEQ ID NO: 354 - protein, 464 aa, synthetic construct
EVQLVESGGGLVQPGGSLRLSCAASGFNIYYYSMHWVRQAPGKGLEWVASISPYYSYTSYADSVKGRFTISADTSKNTAYLQMNSLRAEDTAVYYCARYGYYGWDYHRYSAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGSHHHHHH

SEQ ID NO: 355 - protein, 125 aa, synthetic construct
EVQLVESGGGLVQPGGSLRLSCAASGFNIYYYSMHWVRQAPGKGLEWVASISPYYSYTSYADSVKGRFTISADTSKNTAYLQMNSLRAEDTAVYYCARYGYYGWDYHRYSAFDYWGQGTLVTVSS

SEQ ID NO: 356 - protein, 212 aa, synthetic construct
DIQMTQSPSSLSASVGDRVTITCRASQSVGSALAWYQQKPGKAPKLLIYSASSLYSGVPSRFSGSRSGTDFTLTISSLQPEDFATYYCQQAYPITFGQGTKVEIKRTVAAPSVFIFPPSDSQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 357 - protein, 105 aa, synthetic construct
DIQMTQSPSSLSASVGDRVTITCRASQSVGSALAWYQQKPGKAPKLLIYSASSLYSGVPSRFSGSRSGTDFTLTISSLQPEDFATYYCQQAYPITFGQGTKVEIK

SEQ ID NO: 358 - protein, 454 aa, synthetic construct
EVQLVESGGGLVQPGGSLRLSCAASGFNLYYSYIHWVRQAPGKGLEWVASIYPSYGSTYYADSVKGRFTISRDNSKNTLYLQMNSLRAEDTAVYYCARGYAIDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGSHHHHHH

SEQ ID NO: 359 - protein, 115 aa, synthetic construct
EVQLVESGGGLVQPGGSLRLSCAASGFNLYYSYIHWVRQAPGKGLEWVASIYPSYGSTYYADSVKGRFTISRDNSKNTLYLQMNSLRAEDTAVYYCARGYAIDYWGQGTLVTVSS

SEQ ID NO: 360 - protein, 213 aa, synthetic construct
DIQMTQSPSSLSASVGDRVTITCRASQSVGSALAWYQQKPGKAPKLLIYSASSLYSGVPSRFSGSRSGTDFTLTISSLQPEDFATYYCQQSYYPITFGQGTKVEIKRTVAAPSVFIFPPSDSQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 361 - protein, 106 aa, synthetic construct
DIQMTQSPSSLSASVGDRVTITCRASQSVGSALAWYQQKPGKAPKLLIYSASSLYSGVPSRFSGSRSGTDFTLTISSLQPEDFATYYCQQSYYPITFGQGTKVEIK

SEQ ID NO: 362 - protein, 368 aa, synthetic construct
EVQLVESGGGLVQPGGSLRLSCAASGFRIYSSSYYIGWVRRAPGKGEELVARIYPSSGSTYYADSVKGRFTISADTSKNTAYLQMNSLRAEDTAVYYCARYAVGYGYPWYGWGLDYWGQGTLVTVSSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGSHHHHHH

SEQ ID NO: 363 - protein, 127 aa, synthetic construct
EVQLVESGGGLVQPGGSLRLSCAASGFRIYSSSYYIGWVRRAPGKGEELVARIYPSSGSTYYADSVKGRFTISADTSKNTAYLQMNSLRAEDTAVYYCARYAVGYGYPWYGWGLDYWGQGTLVTVSS

SEQ ID NO: 364 - protein, 459 aa, synthetic construct
EVQLVESGGGLVQPGGSLRLSCAASGFNIYYYSIHWVRQAPGKGLEWVASIYSSSGYTSYADSVKGRFTISADTSKNTAYLQMNSLRAEDTAVYYCARYPYWYFDGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGSHHHHHH

SEQ ID NO: 365 - protein, 120 aa, synthetic construct
EVQLVESGGGLVQPGGSLRLSCAASGFNIYYYSIHWVRQAPGKGLEWVASIYSSSGYTSYADSVKGRFTISADTSKNTAYLQMNSLRAEDTAVYYCARYPYWYFDGFDYWGQGTLVTVSS

SEQ ID NO: 366 - protein, 214 aa, synthetic construct
DIQMTQSPSSLSASVGDRVTITCRASQSVGSALAWYQQKPGKAPKLLIYSASSLYSGVPSRFSGSRSGTDFTLTISSLQPEDFATYYCQQGYSDLITFGQGTKVEIKRTVAAPSVFIFPPSDSQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 367 - protein, 107 aa, synthetic construct
DIQMTQSPSSLSASVGDRVTITCRASQSVGSALAWYQQKPGKAPKLLIYSASSLYSGVPSRFSGSRSGTDFTLTISSLQPEDFATYYCQQGYSDLITFGQGTKVEIK

SEQ ID NO: 368 - protein, 456 aa, synthetic construct
EVQLLESGGGLVQPGGSLRLSCAASGFTFSSYAMSWVRQAPGKGLEWVSAINSQGKSTYYADSVKGRFTISRDNSKNTLYLQMNSLRAEDTAVYYCARWGDEGFDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGSHHHHHH

SEQ ID NO: 369 - protein, 117 aa, synthetic construct; identical to SEQ ID NO: 31

SEQ ID NO: 370 - protein, 214 aa, synthetic construct; identical to SEQ ID NO: 32

SEQ ID NO: 371 - protein, 107 aa, synthetic construct; identical to SEQ ID NO: 33

SEQ ID NO: 372 - protein, 460 aa, synthetic construct
EVQLVESGGGLVQPGGSLRLSCAASGFTLSGDWIHWVRQAPGKGLEWVGEISAAGGYTDYADSVKGRFTISADTSKNTAYLQMNSLRAEDTAVYYCARESRVSFEAAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGSHHHHHH

SEQ ID NO: 373 - protein, 121 aa, synthetic construct [sequence omitted]

SEQ ID NO: 374 - protein, 214 aa, synthetic construct; identical to SEQ ID NO: 308

SEQ ID NO: 375 - protein, 107 aa, synthetic construct [sequence omitted]

SEQ ID NO: 376 - protein, 459 aa, synthetic construct
EVQLQESGPGLVKPGGSLSLSCAASGFVFSSYDMSWVRQTPERGLEWVAYISSGGGITYAPSTVKGRFTVSRDNAKNTLYLQMNSLTSEDTAVYYCAAHYFGSSGPFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGSHHHHHH

SEQ ID NO: 377 - protein, 120 aa, synthetic construct [sequence omitted]

SEQ ID NO: 378 - protein, 214 aa, synthetic construct; identical to SEQ ID NO: 272

SEQ ID NO: 379 - protein, 107 aa, synthetic construct [sequence omitted]

SEQ ID NO: 380 - protein, 457 aa, synthetic construct
EVQLLESGGGLVQPGGSLRLSCAASGFTFSNYAMSWVRQAPGKGLEWVSSISGSGDYTYYTDSVKGRFTISRDNSKNTLYLQMNSLRAEDTAVYYCARSPWGYYLDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGSHHHHHH

SEQ ID NO: 381 - protein, 118 aa, synthetic construct; identical to SEQ ID NO: 295

SEQ ID NO: 382 - protein, 214 aa, synthetic construct; identical to SEQ ID NO: 296

SEQ ID NO: 383 - protein, 107 aa, synthetic construct; identical to SEQ ID NO: 297

SEQ ID NO: 384 - protein, 458 aa, synthetic construct
EVQLVESGGGVVQPGRSLRLSCSASGFDFTTYWMSWVRQAPGKGLEWIGEIHPDSSTINYAPSLKDRFTISRDNAKNTLFLQMDSLRPEDTGVYFCASLYFGFPWFAYWGQGTPVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGSHHHHHH

SEQ ID NO: 385 - protein, 119 aa, synthetic construct; identical to SEQ ID NO: 263

SEQ ID NO: 386 - protein, 213 aa, synthetic construct; identical to SEQ ID NO: 264

SEQ ID NO: 387 - protein, 106 aa, synthetic construct; identical to SEQ ID NO: 265

SEQ ID NO: 388 - protein, 458 aa, synthetic construct
EVQLVESGGGLVKPGGSLRLSCAASGFTFSNAWMSWVRQAPGKGLEWVGRIKSKTDGGTTDYAAPVQGRFTISRDDSKNTLYLQMNSLKTEDTAVYYCTIFGVVSFDYWGQGTLVTVSSASTKGPSVLPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGSHHHHHH

SEQ ID NO: 389 - protein, 119 aa, synthetic construct
EVQLVESGGGLVKPGGSLRLSCAASGFTFSNAWMSWVRQAPGKGLEWVGRIKSKTDGGTTDYAAPVQGRFTISRDDSKNTLYLQMNSLKTEDTAVYYCTIFGVVSFDYWGQGTLVTVSS

SEQ ID NO: 390 - protein, 214 aa, synthetic construct
DVQMTQSPSSLSASVGDRVTITCRASQSISDYLSWYQQRPGKAPNLLIYAASNLKTGVPSRESGSGSGTDFTLTISTLQPEDFATYYCQQSYRSPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 391 - protein, 107 aa, synthetic construct
DVQMTQSPSSLSASVGDRVTITCRASQSISDYLSWYQQRPGKAPNLLIYAASNLKTGVPSRESGSGSGTDFTLTISTLQPEDFATYYCQQSYRSPWTFGQGTKVEIK

SEQ ID NO: 392-393 - skipped (2 entries)

SEQ ID NO: 394 - protein, 458 aa, synthetic construct
EVQLVESGGDRVQPGRSLTLSCVTSGFTFNNYWMTWIRQVPGKGLEWVASISSSGGSIYYPDSVKGRFTISRDNAKNTLYLQMNSLRSEDTATYYCARDERWAGAMDAWGQGTSVTVSSASTKGPSVLPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGSHHHHHH

SEQ ID NO: 395 - protein, 119 aa, synthetic construct
EVQLVESGGDRVQPGRSLTLSCVTSGFTFNNYWMTWIRQVPGKGLEWVASISSSGGSIYYPDSVKGRFTISRDNAKNTLYLQMNSLRSEDTATYYCARDERWAGAMDAWGQGTSVTVSS

SEQ ID NO: 396 - protein, 214 aa, synthetic construct
DIQMTQSPSSVLSASVGDRVTLSCKASQNINKNLDWYQQKHGEAPKLLIYETNNLQTGIPSRFSGSGSGTDYTLTISSLQPEDVATYYCYQHNSRFTFGSGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 397 - protein, 107 aa, synthetic construct
DIQMTQSPSSVLSASVGDRVTLSCKASQNINKNLDWYQQKHGEAPKLLIYETNNLQTGIPSRFSGSGSGTDYTLTISSLQPEDVATYYCYQHNSRFTFGSGTKLEIK

SEQ ID NO: 398 - protein, 459 aa, synthetic construct
EVQLVESGGGLVQPGGSLRLSCAASGFSFSDFAMSWVRQAPGKGLEWVATIGRVAFHTYYPDSMKGRFTISRDNSKNTLYLQMNSLRAEDTAVYYCARHRGFDVGHFDFWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGSHHHHHH

SEQ ID NO: 399 - protein, 120 aa, synthetic construct
EVQLVESGGGLVQPGGSLRLSCAASGFSFSDFAMSWVRQAPGKGLEWVATIGRVAFHTYYPDSMKGRFTISRDNSKNTLYLQMNSLRAEDTAVYYCARHRGFDVGHFDFWGQGTLVTVSS

SEQ ID NO: 400 - protein, 219 aa, synthetic construct
DIQMTQSPSSLSASVGDRVTITCRSSETLVHSSGNTYLEWYQQKPGKAPKLLIYRVSNRFSGVPSRFSGSGSGTDFTLTISSLQPEDFATYYCFQGSFNPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 401 - protein, 112 aa, synthetic construct
DIQMTQSPSSLSASVGDRVTITCRSSETLVHSSGNTYLEWYQQKPGKAPKLLIYRVSNRFSGVPSRFSGSGSGTDFTLTISSLQPEDFATYYCFQGSFNPLTFGQGTKVEIK

SEQ ID NO: 402 - protein, 456 aa, synthetic construct
QVQLVQSGAEVKKPGASVKVSCKASGYSFSGYFMNWVRQAPGQGLEWMGLINPYNGDSFYNQKFKGRVTMTRDTSTSTVYMELSSLRSEDTAVYYCARGLRRDFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGSHHHHHH

SEQ ID NO: 403 - protein, 117 aa, synthetic construct
QVQLVQSGAEVKKPGASVKVSCKASGYSFSGYFMNWVRQAPGQGLEWMGLINPYNGDSFYNQKFKGRVTMTRDTSTSTVYMELSSLRSEDTAVYYCARGLRRDFDYWGQGTLVTVSS

SEQ ID NO: 404 - protein, 219 aa, synthetic construct; identical to SEQ ID NO: 288

SEQ ID NO: 405 - protein, 112 aa, synthetic construct; identical to SEQ ID NO: 289

SEQ ID NO: 406 - protein, 458 aa, synthetic construct
EVQLVESGGGVVQPGRSLRLSCSASGFTFSGYGLSWVRQAPGKGLEWVAMISSGGSYTYYADSVKGRFAISRDNAKNTLFLQMDSLRPEDTGVYFCARHGDDPAWFAYWGQGTPVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGSHHHHHH

SEQ ID NO: 407 - protein, 119 aa, synthetic construct
EVQLVESGGGVVQPGRSLRLSCSASGFTFSGYGLSWVRQAPGKGLEWVAMISSGGSYTYYADSVKGRFAISRDNAKNTLFLQMDSLRPEDTGVYFCARHGDDPAWFAYWGQGTPVTVSS

SEQ ID NO: 408 - protein, 217 aa, synthetic construct
DIQLTQSPSSLSASVGDRVTITCSVSSSISSNNLHWYQQKPGKAPKPWIYGTSNLASGVPSRFSGSGSGTDYTFTISSLQPEDIATYYCQQWSSYPYMYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 409 - protein, 110 aa, synthetic construct
DIQLTQSPSSLSASVGDRVTITCSVSSSISSNNLHWYQQKPGKAPKPWIYGTSNLASGVPSRFSGSGSGTDYTFTISSLQPEDIATYYCQQWSSYPYMYTFGQGTKVEIK

SEQ ID NO: 410 - protein, 457 aa, synthetic construct
QVQLVQSGAEVVKPGASVKISCKASGYTFTGYFMNWVKQSPGQSLEWIGRIHPYDGDTFYNQKFQGKATLTVDKSSNTAHMELLSLTSEDFAVYYCTRYDGSRAMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGSHHHHHH

SEQ ID NO: 411 - protein, 118 aa, synthetic construct
QVQLVQSGAEVVKPGASVKISCKASGYTFTGYFMNWVKQSPGQSLEWIGRIHPYDGDTFYNQKFQGKATLTVDKSSNTAHMELLSLTSEDFAVYYCTRYDGSRAMDYWGQGTTVTVSS

SEQ ID NO: 412 - protein, 218 aa, synthetic construct
DIVLTQSPLSLAVSLGQPAIISCKASQSVSFAGTSLMHWYHQKPGQQPRLLIYRASNLEAGVPDRFSGSGSKTDFTLTISPVEAEDAATYYCQQSREYPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 413 - protein, 111 aa, synthetic construct
DIVLTQSPLSLAVSLGQPAIISCKASQSVSFAGTSLMHWYHQKPGQQPRLLIYRASNLEAGVPDRFSGSGSKTDFTLTISPVEAEDAATYYCQQSREYPYTFGGGTKLEIK

SEQ ID NO: 414 - protein, 456 aa, synthetic construct
QVQLVESGGGLVKPGGSLRLSCAASGFTFSNYYMSWVRQAPGKGLEWISYISGRGSTIFYADSVKGRITISRDNAKNSLFLQMNSLRAEDTAVYFCVKDRGGYSPYWGQGTLVTVSSASTKGPSVLPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGSHHHHHH

SEQ ID NO: 415 - protein, 117 aa, synthetic construct
QVQLVESGGGLVKPGGSLRLSCAASGFTFSNYYMSWVRQAPGKGLEWISYISGRGSTIFYADSVKGRITISRDNAKNSLFLQMNSLRAEDTAVYFCVKDRGGYSPYWGQGTLVTVSS

SEQ ID NO: 416 - protein, 215 aa, synthetic construct
DIQMTQSPSSLSASVGDRVTITCRASQSISTYLNWYQQKPGKAPKLLIYTASSLQSGVPSRFSGSGSGTDFTLTISSLQPEDFATYYCQQSYSTPPITFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 417 - protein, 108 aa, synthetic construct
DIQMTQSPSSLSASVGDRVTITCRASQSISTYLNWYQQKPGKAPKLLIYTASSLQSGVPSRFSGSGSGTDFTLTISSLQPEDFATYYCQQSYSTPPITFGQGTRLEIK

SEQ ID NO: 418 - protein, 461 aa, synthetic construct; identical to SEQ ID NO: 278

SEQ ID NO: 419 - protein, 122 aa, synthetic construct; identical to SEQ ID NO: 279

SEQ ID NO: 420 - protein, 214 aa, synthetic construct
DIQMTQSPSSLSASVGDRVTITCRASQSISSYLNWYQQKPGKAPKLLIYAASSLQSGVPSRFSGSGSGTDFTLTISSLQPEDFATYYCQQSYSTPPTFGQGTKVEIKRTVAAPSVEIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 421 - protein, 107 aa, synthetic construct; identical to SEQ ID NO: 8

SEQ ID NO: 422 - protein, 463 aa, synthetic construct
QVQLQESGPGLVKPSETLSLTCTVSGGSISSSNYYWGWIRQPPGKGLEWIGNIYYRGYTYYNPSLKSRVTISVDTSKKQFSLTLSSVTAADTAMYYCAREGSDYGDYVGAFDIWDQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGSHHHHHH

SEQ ID NO: 423 - protein, 124 aa, synthetic construct; identical to SEQ ID NO: 19

SEQ ID NO: 424 - protein, 214 aa, synthetic construct; identical to SEQ ID NO: 420

SEQ ID NO: 425 - protein, 107 aa, synthetic construct; identical to SEQ ID NO: 8

SEQ ID NO: 426 - protein, 461 aa, synthetic construct; identical to SEQ ID NO: 282

SEQ ID NO: 427 - protein, 122 aa, synthetic construct; identical to SEQ ID NO: 283

SEQ ID NO: 428 - protein, 214 aa, synthetic construct; identical to SEQ ID NO: 420

SEQ ID NO: 429 - protein, 107 aa, synthetic construct; identical to SEQ ID NO: 8

SEQ ID NO: 430 - protein, 451 aa, synthetic construct
EVQLVQSGAEVKKPGASVKVSCKASGYTFTTYTIHWVRQAPGQGLEWMGYINPRSGYTEYNQKFQDRVTMTRDTSTSTVYMELSSLRSEDTAVYYCARSYEFWGQGTTVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGSHHHHHH

SEQ ID NO: 431 - protein, 113 aa, synthetic construct; identical to SEQ ID NO: 23

SEQ ID NO: 432 - protein, 214 aa, synthetic construct
EIVMTQSPATLSVSPGERATLSCKASQNVGINVAWYQQKPGQAPRALIYSASYRYSGIPARFSGSGSGTEFTLTISSLQSEDFAVYYCHQYKTYPYTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 433 - protein, 107 aa, synthetic construct; identical to SEQ ID NO: 25

SEQ ID NO: 434 - protein, 459 aa, synthetic construct
AVQLVESGGGSVQPGRSMRLSCAASGFTFSNYDMTWVRQAPTKGLEWVASITSDGGSTYSRDSVKGRFTISRDNAKSTLYLQMDSLRSEDTATYYCTTDRGRYLPYYFDYWGQGVMVTVSAAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGSHHHHHH

SEQ ID NO: 435 - protein, 121 aa, synthetic construct
AVQLVESGGGSVQPGRSMRLSCAASGFTFSNYDMTWVRQAPTKGLEWVASITSDGGSTYSRDSVKGRFTISRDNAKSTLYLQMDSLRSEDTATYYCTTDRGRYLPYYFDYWGQGVMVTVSA

SEQ ID NO: 436 - protein, 218 aa, synthetic construct
DVVLTQTPVSLSVTVGDQASISCRSSQSLEYSDGYSYLEWYLQKPGQSPQLLIYEVSSRFSGVPDRFIGSGSGTDFTLKISRVEPEDLGVYYCFQAIHDPTFGAGTKLELKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 437 - protein, 111 aa, synthetic construct; identical to SEQ ID NO: 29

SEQ ID NO: 438 - protein, 457 aa, synthetic construct
QVQLKESGPGLVQPSQTLSLTCTVSGFSLTTYSVHWVRQHSGKNLEWMGRMWTAGDTSYNSAFTSRLNIFRDTSKSQVFLKMNSLQTEDTGTYYCARSSYTSGYPFDSWGQGVMVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGSHHHHHH

SEQ ID NO: 439 - protein, 119 aa, synthetic construct; identical to SEQ ID NO: 275

SEQ ID NO: 440 - protein, 212 aa, synthetic construct
DTVLTQSPALAVSPGERVTISCRASESVSKLMHWYQQRPGQQPQLLIYLTSHLASGVPARFSGSGSGTDFTLTIDPVEADDTATYYCQQSRNDPTFGAGTKLELKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 441 - protein, 105 aa, synthetic construct; identical to SEQ ID NO: 277

SEQ ID NO: 442 - protein, 455 aa, synthetic construct
EVQLVESGGGLVPPGKSLKLSCSASGFPFSNYGMHWIRQAPGKGLDWVGYISSNSGTIYADAVKGRFTISRDNAKNTLYLLINSLKSEDTAMYYCARGYFDGYYRFWGQGVMVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGSHHHHHH

SEQ ID NO: 443 - protein, 117 aa, synthetic construct
EVQLVESGGGLVPPGKSLKLSCSASGFPFSNYGMHWIRQAPGKGLDWVGYISSNSGTIYADAVKGRFTISRDNAKNTLYLLINSLKSEDTAMYYCARGYFDGYYRFWGQGVMVTVSS

SEQ ID NO: 444 - protein, 219 aa, synthetic construct
DIVMTQSPFSLAVSEGDMVTIMCRSSQSLLSSGNQKNYLAWYQQKPGQSPKLLIYHASTRQSGVPDRFIGSGSGTDFTLTISDVQAEDLADYYCLQHYSSPTFGSGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 445 - protein, 112 aa, synthetic construct
DIVMTQSPFSLAVSEGDMVTIMCRSSQSLLSSGNQKNYLAWYQQKPGQSPKLLIYHASTRQSGVPDRFIGSGSGTDFTLTISDVQAEDLADYYCLQHYSSPTFGSGTKLEIK

SEQ ID NO: 446 - protein, 459 aa, synthetic construct
EVHLVESGGGLVQPGGSLKLSCAASGFTFSNYDMAWVRQAPTRGLEWVASISPGGGKTYYRDSVKGRLTISRNNAENTQYLQIDSLRSEDTATYYCSRLGPAYSGEWFAYWGQGTLVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGSHHHHHH

SEQ ID NO: 447 - protein, 121 aa, synthetic construct
EVHLVESGGGLVQPGGSLKLSCAASGFTFSNYDMAWVRQAPTRGLEWVASISPGGGKTYYRDSVKGRLTISRNNAENTQYLQIDSLRSEDTATYYCSRLGPAYSGEWFAYWGQGTLVTVSS

SEQ ID NO: 448 - protein, 213 aa, synthetic construct
DTVLTQSPALTVSPGDKITISCRASEGVNTRIHWYQQKSGQQPKLLIYGASNLDSGVPDRFSGSGFGTDFTLTIDPVEASDTATYFCQQSWNVPHTFGGGTKLELKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 449 - protein, 106 aa, synthetic construct
DTVLTQSPALTVSPGDKITISCRASEGVNTRIHWYQQKSGQQPKLLIYGASNLDSGVPDRFSGSGFGTDFTLTIDPVEASDTATYFCQQSWNVPHTFGGGTKLELK

SEQ ID NO: 450 - protein, 458 aa, synthetic construct
EVQLAESGGGLEQPGRSLKLSCAASGFTFSNYDMAWVRQAPTKGLEWVASIIKSGDTSYYRDSVKGRFTVSRDNAKSTLYLQMDSLRSEDTATYYCARHGVGSYDWFADWGQGTLVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGSHHHHHH

SEQ ID NO: 451 - protein, 120 aa, synthetic construct
EVQLAESGGGLEQPGRSLKLSCAASGFTFSNYDMAWVRQAPTKGLEWVASIIKSGDTSYYRDSVKGRFTVSRDNAKSTLYLQMDSLRSEDTATYYCARHGVGSYDWFADWGQGTLVTVSS

SEQ ID NO: 452 - protein, 216 aa, synthetic construct
DIVLTQSPALAVSLGQRATISCRASQSVTISGFNLMHWYQQKPGQQPKLLIYRASNLAFGIPARFSGSGSGTDFTLTINPVQADDFTTYYCQQSRKSRTFGGGTKLELKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 453 - protein, 109 aa, synthetic construct
DIVLTQSPALAVSLGQRATISCRASQSVTISGFNLMHWYQQKPGQQPKLLIYRASNLAFGIPARFSGSGSGTDFTLTINPVQADDFTTYYCQQSRKSRTFGGGTKLELK

SEQ ID NO: 454 - protein, 456 aa, synthetic construct; identical to SEQ ID NO: 290

SEQ ID NO: 455 - protein, 117 aa, synthetic construct [sequence omitted]

SEQ ID NO: 456 - protein, 220 aa, synthetic construct; identical to SEQ ID NO: 292

SEQ ID NO: 457 - protein, 113 aa, synthetic construct [sequence omitted]

SEQ ID NO: 458 - protein, 457 aa, synthetic construct
QVTLRESGPALVKPTQTLTLTCTFSGFSLSTYGIGVGWIRQPPGKALEWLAHIWWNDNKYYSTSLKTRLTISKDTSKNQVVLTMTNMDPVDTATYYCARISLPYFDYWGQGTTLTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGSHHHHHH

SEQ ID NO: 459 - protein, 118 aa, synthetic construct
QVTLRESGPALVKPTQTLTLTCTFSGFSLSTYGIGVGWIRQPPGKALEWLAHIWWNDNKYYSTSLKTRLTISKDTSKNQVVLTMTNMDPVDTATYYCARISLPYFDYWGQGTTLTVSS

SEQ ID NO: 460 - protein, 214 aa, synthetic construct
DIQLTQSPSFLSASVGDRVTITCKASQNVGTAVAWYQQKPGKAPKLLIYSASNRYTGVPSRFSGSGSGTEFTLTISSLQPEDFATYYCQQYSSYPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 461 - protein, 107 aa, synthetic construct
DIQLTQSPSFLSASVGDRVTITCKASQNVGTAVAWYQQKPGKAPKLLIYSASNRYTGVPSRFSGSGSGTEFTLTISSLQPEDFATYYCQQYSSYPLTFGGGTKVEIK

SEQ ID NO: 462 - protein, 456 aa, synthetic construct
EVQLVESGGGLVQPGGSMRLSCVASGFPFSNYWMNWVRQAPGKGLEWVGEIRLKSNNYTTHYAESVKGRFTISRDDSKNSLYLQMNSLKTEDTAVYYCTRHYYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGSHHHHHH

SEQ ID NO: 463 - protein, 117 aa, synthetic construct
EVQLVESGGGLVQPGGSMRLSCVASGFPFSNYWMNWVRQAPGKGLEWVGEIRLKSNNYTTHYAESVKGRFTISRDDSKNSLYLQMNSLKTEDTAVYYCTRHYYFDYWGQGTLVTVSS

SEQ ID NO: 464 - protein, 219 aa, synthetic construct; identical to SEQ ID NO: 56

SEQ ID NO: 465 - protein, 112 aa, synthetic construct; identical to SEQ ID NO: 57

SEQ ID NO: 466 - protein, 461 aa, synthetic construct; identical to SEQ ID NO: 258

SEQ ID NO: 467 - protein, 122 aa, synthetic construct; identical to SEQ ID NO: 259

SEQ ID NO: 468 - protein, 214 aa, synthetic construct; identical to SEQ ID NO: 260

SEQ ID NO: 469 - protein, 107 aa, synthetic construct; identical to SEQ ID NO: 261

SEQ ID NO: 470 - protein, 460 aa, synthetic construct
QVQLVQSGAEVKKPGASVKVSCKASGYTFTDYAMHWVRQAPGQRLEWMGLISTYSGDTKYNQNFQGRVTMTVDKSASTAYMELSSLRSEDTAVYYCARGDYSGSRYWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGSHHHHHH

SEQ ID NO: 471 - protein, 121 aa, synthetic construct
QVQLVQSGAEVKKPGASVKVSCKASGYTFTDYAMHWVRQAPGQRLEWMGLISTYSGDTKYNQNFQGRVTMTVDKSASTAYMELSSLRSEDTAVYYCARGDYSGSRYWFAYWGQGTLVTVSS

SEQ ID NO: 472 - protein, 214 aa, synthetic construct
DIQMTQSPSSLSASVGDRVTITCQASENIYGALNWYQRKPGKSPKLLIYGASNLATGMPSRFSGSGSGTDYTFTISSLQPEDIATYYCQQVLSSPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 473 - protein, 107 aa, synthetic construct
DIQMTQSPSSLSASVGDRVTITCQASENIYGALNWYQRKPGKSPKLLIYGASNLATGMPSRFSGSGSGTDYTFTISSLQPEDIATYYCQQVLSSPYTFGGGTKLEIK

SEQ ID NO: 474 - protein, 460 aa, synthetic construct
QITLKESGPTLVKPTQTLTLTCTFSGFSLSTYGMGVGWIRQPPGKALEWLANIWWSEDKHYSPSLKSRLTITKDTSKNQVVLTITNVDPVDTATYYCVQIDYGNDYAFTYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGSHHHHHH

SEQ ID NO: 475 - protein, 121 aa, synthetic construct
QITLKESGPTLVKPTQTLTLTCTFSGFSLSTYGMGVGWIRQPPGKALEWLANIWWSEDKHYSPSLKSRLTITKDTSKNQVVLTITNVDPVDTATYYCVQIDYGNDYAFTYWGQGTLVTVSS

SEQ ID NO: 476 - protein, 219 aa, synthetic construct
DIVMTQSPLSLPVTPGEPASISCRSSKSLLHSNGITYLYWYLQKPGQSPQLLIYQMSNLASGVPDRFSGSGSGTDFTLKISRVEAEDVGVYYCAQNLELPYTFGQGTKLEIKRTVAAPSVFIFPPSDEQLKSGTASVVCLLNNTYPREAKVQWKVDNALQSGNSQESVTEQDSKDSTYSLSSTLTLSKADYEKHKVYACEVTHQGLSSPVTKSFNRGEC

SEQ ID NO: 477 - protein, 112 aa, synthetic construct
DIVMTQSPLSLPVTPGEPASISCRSSKSLLHSNGITYLYWYLQKPGQSPQLLIYQMSNLASGVPDRFSGSGSGTDFTLKISRVEAEDVGVYYCAQNLELPYTFGQGTKLEIK

SEQ ID NO: 478 - protein, 459 aa, synthetic construct
EVQLVQSGAEVKKPGSSVKVSCKASGYTFTTYWMHWVRQAPGQGLEWIGEIDPSDSYSNYNQKFKDRATLTVDKSTSTAYMELSSLRSEDTAVYYCARNGGLGPAWFSYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGSHHHHHH

SEQ ID NO: 479 - protein, 120 aa, synthetic construct
EVQLVQSGAEVKKPGSSVKVSCKASGYTFTTYWMHWVRQAPGQGLEWIGEIDPSDSYSNYNQKFKDRATLTVDKSTSTAYMELSSLRSEDTAVYYCARNGGLGPAWFSYWGQGTLVTVSS

SEQ ID NO: 480 - protein, 214 aa, synthetic construct
DIQMTQSPSSVSASVGDRVTITCKASQYVGTAVAWYQQKPGKSPKLLIYSASNRYTGVPSRFSDSGSGTDFTLTISSLQPEDFATYFCQQYSSYPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 481 - protein, 107 aa, synthetic construct
DIQMTQSPSSVSASVGDRVTITCKASQYVGTAVAWYQQKPGKSPKLLIYSASNRYTGVPSRFSDSGSGTDFTLTISSLQPEDFATYFCQQYSSYPWTFGGGTKVEIK

SEQ ID NO: 482 - protein, 459 aa, synthetic construct
QVQLVQSGAEVVKPGASVKMSCKASGYTFTGYNIHWVKQAPGQGLEWIGAIYPGNGDTSYKQKFRGRATLTADTSTSTVYMELSSLRSEDSAVYYCARGETARATFAYWGQGTLVTVSSGASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGSHHHHHH

SEQ ID NO: 483 - protein, 119 aa, synthetic construct
QVQLVQSGAEVVKPGASVKMSCKASGYTFTGYNIHWVKQAPGQGLEWIGAIYPGNGDTSYKQKFRGRATLTADTSTSTVYMELSSLRSEDSAVYYCARGETARATFAYWGQGTLVTVSS

SEQ ID NO: 484 - protein, 215 aa, synthetic construct
DIQMTQSPSSLSASVGDRVTITCSASQDIGNFLNWYQQKPGKTVKVLIYYTSSLYSGVPSRFSGSGSGTDYTLTISSLQPEDFATYYCQQYSKLPLTFGQGTKLELKRRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 485 - protein, 108 aa, synthetic construct
DIQMTQSPSSLSASVGDRVTITCSASQDIGNFLNWYQQKPGKTVKVLIYYTSSLYSGVPSRFSGSGSGTDYTLTISSLQPEDFATYYCQQYSKLPLTFGQGTKLELKR

SEQ ID NO: 486 - protein, 456 aa, synthetic construct
QVQLVQSGAEVKKPGASVKVSCKASGYTFTGYFMNWVRQAPGQGLEWMGLINPYNGDSFYNQKFKGRVTMTRDTSTSTVYMELSSLRSEDTAVYYCARGLRRDFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGSHHHHHH

SEQ ID NO: 487 - protein, 117 aa, synthetic construct
QVQLVQSGAEVKKPGASVKVSCKASGYTFTGYFMNWVRQAPGQGLEWMGLINPYNGDSFYNQKFKGRVTMTRDTSTSTVYMELSSLRSEDTAVYYCARGLRRDFDYWGQGTLVTVSS

SEQ ID NO: 488 - protein, 219 aa, synthetic construct
DVVMTQSPLSLPVTLGQPASISCKSSQSLLDSDGKTYLNWFQQRPGQSPRRLIYLVSELDSGVPDRFSGSGSGTDFTLKISRVEAEDVGVYYCWQGTHFPRTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 489 - protein, 112 aa, synthetic construct
DVVMTQSPLSLPVTLGQPASISCKSSQSLLDSDGKTYLNWFQQRPGQSPRRLIYLVSELDSGVPDRFSGSGSGTDFTLKISRVEAEDVGVYYCWQGTHFPRTFGGGTKLEIK

SEQ ID NO: 490 - protein, 456 aa, synthetic construct; identical to SEQ ID NO: 286

SEQ ID NO: 491 - protein, 117 aa, synthetic construct; identical to SEQ ID NO: 287

SEQ ID NO: 492 - protein, 219 aa, synthetic construct; identical to SEQ ID NO: 288

SEQ ID NO: 493 - protein, 112 aa, synthetic construct; identical to SEQ ID NO: 289

SEQ ID NO: 494 - protein, 456 aa, synthetic construct
QVQLVQSGAEVKKPGASVKVSCKASGYSFSGYFMNWVRQAPGQGLEWMGLINPYNGDSFYNQKFKGRVTMTRDKSSSTAYMELSSLRSEDTAVYYCARGLRRDFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGSHHHHHH

SEQ ID NO: 495 - protein, 117 aa, synthetic construct
QVQLVQSGAEVKKPGASVKVSCKASGYSFSGYFMNWVRQAPGQGLEWMGLINPYNGDSFYNQKFKGRVTMTRDKSSSTAYMELSSLRSEDTAVYYCARGLRRDFDYWGQGTLVTVSS

SEQ ID NO: 496 - protein, 219 aa, synthetic construct; identical to SEQ ID NO: 288

SEQ ID NO: 497 - protein, 112 aa, synthetic construct; identical to SEQ ID NO: 289

SEQ ID NO: 498 - protein, 457 aa, synthetic construct
QVQLVQSGAEVKKPGASVKMSCKASGYTFTSYWMHWVRQAPGQGLEWIGAIYPGNSETGYAQKFQGRATLTADTSTSTAYMELSSLRSEDTAVYYCTRENWDPGFAFWGQGTLITVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGSHHHHHH

SEQ ID NO: 499 - protein, 118 aa, synthetic construct; identical to SEQ ID NO: 303

SEQ ID NO: 500 - protein, 213 aa, synthetic construct; identical to SEQ ID NO: 304

SEQ ID NO: 501 - protein, 106 aa, synthetic construct; identical to SEQ ID NO: 305

SEQ ID NO: 502 - protein, 458 aa, synthetic construct
EVQLLESGGGLVQPGGSLRLSCAASGFTFSHYVMAWVRQAPGKGLEWVSSISSSGGWTLYADSVKGRFTISRDNSKNTLYLQMNSLRAEDTAVYYCTRGLKMATIFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGSHHHHHH

SEQ ID NO: 503 - protein, 119 aa, synthetic construct [sequence omitted]

SEQ ID NO: 504 - protein, 217 aa, synthetic construct; identical to SEQ ID NO: 224

SEQ ID NO: 505 - protein, 111 aa, synthetic construct [sequence omitted]

SEQ ID NO: 506 - protein, 455 aa, synthetic construct
EVQLVESGGGLVQPGGSLRLSCAASGYSITNDYAWNWVRQAPGKGLEWVGYISYSGYTTYNPSLKSRFTISRDTSKNTLYLQMNSLRAEDTAVYYCARWTSGLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGSHHHHHH

SEQ ID NO: 507 - protein, 116 aa, synthetic construct
EVQLVESGGGLVQPGGSLRLSCAASGYSITNDYAWNWVRQAPGKGLEWVGYISYSGYTTYNPSLKSRFTISRDTSKNTLYLQMNSLRAEDTAVYYCARWTSGLDYWGQGTLVTVSS

SEQ ID NO: 508 - protein, 214 aa, synthetic construct
DIQMTQSPSSLSASVGDRVTITCKASDLIHNWLAWYQQKPGKAPKLLIYGATSLETGVPSRFSGSGSGTDFTLTISSLQPEDFATYYCQQYWTTPFTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 509 - protein, 107 aa, synthetic construct
DIQMTQSPSSLSASVGDRVTITCKASDLIHNWLAWYQQKPGKAPKLLIYGATSLETGVPSRFSGSGSGTDFTLTISSLQPEDFATYYCQQYWTTPFTFGQGTKVEIK

SEQ ID NO: 510 - protein, 458 aa, synthetic construct
QVTLRESGPALVKPTQTLTLTCTFSGFSLSTAGMSVGWIRQPPGKALEWLADIWWDDKKHYNPSLKDRLTISKDTSKNQVVLKVTNMDPADTATYYCARDMIFNFYFDVWGQGTTVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGSHHHHHH

SEQ ID NO: 511 - protein, 120 aa, synthetic construct [sequence omitted]

SEQ ID NO: 512 - protein, 213 aa, synthetic construct
DIQMTQSPSTLSASVGDRVTITCSASSRVGYMHWYQQKPGKAPKLLIYDTSKLASGVPSRFSGSGSGTEFTLTISSLQPDDFATYYCFQGSGYPFTFGGGTKVEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 513 - protein, 106 aa, synthetic construct [sequence omitted]

SEQ ID NO: 514 - protein, 459 aa, synthetic construct
EVQLVQSGAEVKKPGASVKVSCKASGYKFSSYWIEWVKQAPGQGLEWIGEILPGSDTTNYNEKFKDRATFTSDTSINTAYMELSRLRSDDTAVYYCARDRGNYRAWFGYWGQGTLVTVSSASTKGPSVLPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGSHHHHHH

SEQ ID NO: 515 - protein, 120 aa, synthetic construct
EVQLVQSGAEVKKPGASVKVSCKASGYKFSSYWIEWVKQAPGQGLEWIGEILPGSDTTNYNEKFKDRATFTSDTSINTAYMELSRLRSDDTAVYYCARDRGNYRAWFGYWGQGTLVTVSS

SEQ ID NO: 516 - protein, 214 aa, synthetic construct
DIQMTQSPSSLSASVGDRVTITCRASQDISNYLNWYQQKPGGAVKFLIYYTSRLHSGVPSRFSGSGSGTDYTLTISSLQPEDFATYFCQQGEALPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 517 - protein, 107 aa, synthetic construct
DIQMTQSPSSLSASVGDRVTITCRASQDISNYLNWYQQKPGGAVKFLIYYTSRLHSGVPSRFSGSGSGTDYTLTISSLQPEDFATYFCQQGEALPWTFGGGTKVEIK

SEQ ID NO: 518 - protein, 461 aa, synthetic construct; identical to SEQ ID NO: 278

SEQ ID NO: 519 - protein, 122 aa, synthetic construct; identical to SEQ ID NO: 279

SEQ ID NO: 520 - protein, 214 aa, synthetic construct; identical to SEQ ID NO: 420

SEQ ID NO: 521 - protein, 107 aa, synthetic construct; identical to SEQ ID NO: 8

SEQ ID NO: 522 - protein, 461 aa, synthetic construct; identical to SEQ ID NO: 282

SEQ ID NO: 523 - protein, 122 aa, synthetic construct; identical to SEQ ID NO: 283

SEQ ID NO: 524 - protein, 214 aa, synthetic construct; identical to SEQ ID NO: 420

SEQ ID NO: 525 - protein, 107 aa, synthetic construct; identical to SEQ ID NO: 8

SEQ ID NO: 526 - protein, 449 aa, synthetic construct; identical to SEQ ID NO: 262

SEQ ID NO: 527 - protein, 119 aa, synthetic construct; identical to SEQ ID NO: 263

SEQ ID NO: 528 - protein, 213 aa, synthetic construct; identical to SEQ ID NO: 264

SEQ ID NO: 529 - protein, 106 aa, synthetic construct; identical to SEQ ID NO: 265

SEQ ID NO: 530 - protein, 459 aa, synthetic construct
QVQLVQSGSELKKPGASVKVSCKASGYTFTRYPMNWVRQAPGQGLEWMGWINTNTGNPTYAQGFTGRFVFSLDTSVSTAFLQISSLKAEDTAVYYCARERTNFYDAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGSHHHHHH

SEQ ID NO: 531 - protein, 120 aa, synthetic construct
QVQLVQSGSELKKPGASVKVSCKASGYTFTRYPMNWVRQAPGQGLEWMGWINTNTGNPTYAQGFTGRFVFSLDTSVSTAFLQISSLKAEDTAVYYCARERTNFYDAFDIWGQGTMVTVSS

SEQ ID NO: 532 - protein, 214 aa, synthetic construct; identical to SEQ ID NO: 420

SEQ ID NO: 533 - protein, 107 aa, synthetic construct; identical to SEQ ID NO: 8

SEQ ID NO: 534 - protein, 459 aa, synthetic construct
QVQLVQSGSELKKPGASVKVSCKASGYTFNSYAMDWVRQAPGQGLEWMGWINTNTGNPTYAQAFTGRFVFSLDTSVSTAYLEISSLKAEDTAVYYCARERHGYFEAFDIWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGSHHHHHH

SEQ ID NO: 535 - protein, 120 aa, synthetic construct
QVQLVQSGSELKKPGASVKVSCKASGYTFNSYAMDWVRQAPGQGLEWMGWINTNTGNPTYAQAFTGRFVFSLDTSVSTAYLEISSLKAEDTAVYYCARERHGYFEAFDIWGQGTTVTVSS

SEQ ID NO: 536 - protein, 214 aa, synthetic construct; identical to SEQ ID NO: 420

SEQ ID NO: 537 - protein, 107 aa, synthetic construct; identical to SEQ ID NO: 8

SEQ ID NO: 538 - protein, 459 aa, synthetic construct
EVQLVQSGSELKKPGASVKVSCKASGYTFTKYAMNWVRQVPGQGLEWMGWINTNTGNPTYAQGFTGRFVFSLDTSVRTAYLQISSLKAEDTAVYYCARKGGSYYDWFDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGSHHHHHH

SEQ ID NO: 539 - protein, 120 aa, synthetic construct
EVQLVQSGSELKKPGASVKVSCKASGYTFTKYAMNWVRQVPGQGLEWMGWINTNTGNPTYAQGFTGRFVFSLDTSVRTAYLQISSLKAEDTAVYYCARKGGSYYDWFDPWGQGTLVTVSS

SEQ ID NO: 540 - protein, 214 aa, synthetic construct; identical to SEQ ID NO: 420

SEQ ID NO: 541 - protein, 107 aa, synthetic construct; identical to SEQ ID NO: 8

SEQ ID NO: 542 - protein, 466 aa, synthetic construct
EVQLVQSGSKLKKPGASVKVSCKASGYTFTSYTMNWVRQAPGQGLEWMGWINTDTGDPTYAQGFTGRFVFSLDTSVSTAFLQINSLKAEDTAVYYCARGDCDSTSCYRYSYGYEDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGSHHHHHH

SEQ ID NO: 543 - protein, 127 aa, synthetic construct; identical to SEQ ID NO: 6

SEQ ID NO: 544 - protein, 214 aa, synthetic construct; identical to SEQ ID NO: 420

SEQ ID NO: 545 - protein, 107 aa, synthetic construct; identical to SEQ ID NO: 8

SEQ ID NO: 546 - protein, 460 aa, synthetic construct
QVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYFDVWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGSHHHHHH

SEQ ID NO: 547 - protein, 121 aa, synthetic construct; identical to SEQ ID NO: 15

SEQ ID NO: 548 - protein, 214 aa, synthetic construct; identical to SEQ ID NO: 16

SEQ ID NO: 549 - protein, 107 aa, synthetic construct; identical to SEQ ID NO: 17

SEQ ID NO: 550 - protein, 457 aa, synthetic construct
EVKLVESGGDLVQPGGSLKLSCATSGFTFSDYYMFWVRQTPEKRLEWVAYITNGGDRSYYSDTVTGRFIISRDNAKNTLYLQMSRLKSEDTAMYYCARQGNWAAWFVYWGQGTLVTVSAAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGSHHHHHH

SEQ ID NO: 551 - protein, 119 aa, synthetic construct
EVKLVESGGDLVQPGGSLKLSCATSGFTFSDYYMFWVRQTPEKRLEWVAYITNGGDRSYYSDTVTGRFIISRDNAKNTLYLQMSRLKSEDTAMYYCARQGNWAAWFVYWGQGTLVTVSA

SEQ ID NO: 552 - protein, 219 aa, synthetic construct
DVLMTQTPLSLPVSLGDQASISCRSSHYIVHSDGNTYLEWYLQKPGQSPKLLIYKVSNRFSGVPDRFSGSGSGTDFTLKISRVEAEDLGIYYCFQGSHVPLTFGAGTKLELKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 553 - protein, 112 aa, synthetic construct
DVLMTQTPLSLPVSLGDQASISCRSSHYIVHSDGNTYLEWYLQKPGQSPKLLIYKVSNRFSGVPDRFSGSGSGTDFTLKISRVEAEDLGIYYCFQGSHVPLTFGAGTKLELK

SEQ ID NO: 554 - protein, 451 aa, synthetic construct; identical to SEQ ID NO: 306

SEQ ID NO: 555 - protein, 121 aa, synthetic construct [sequence omitted]

SEQ ID NO: 556 - protein, 214 aa, synthetic construct; identical to SEQ ID NO: 308

SEQ ID NO: 557 - protein, 107 aa, synthetic construct [sequence omitted]

SEQ ID NO: 558 - protein, 448 aa, synthetic construct; identical to SEQ ID NO: 302

SEQ ID NO: 559 - protein, 118 aa, synthetic construct; identical to SEQ ID NO: 303

SEQ ID NO: 560 - protein, 213 aa, synthetic construct; identical to SEQ ID NO: 304

SEQ ID NO: 561 - protein, 106 aa, synthetic construct; identical to SEQ ID NO: 305

SEQ ID NO: 562 - protein, 452 aa, synthetic construct
EVQLVQSGAEVKKPGASVKVSCKASGYTFTTYTIHWVRQAPGQGLEWMGYINPRSGYTEYNQKFQDRVTMTRDTSTSTVYMELSSLRSEDTAVYYCARSYEFWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGSHHHHHH

SEQ ID NO: 563 - protein, 113 aa, synthetic construct; identical to SEQ ID NO: 23

SEQ ID NO: 564 - protein, 214 aa, synthetic construct; identical to SEQ ID NO: 24

SEQ ID NO: 565 - protein, 107 aa, synthetic construct; identical to SEQ ID NO: 25

SEQ ID NO: 566 - protein, 460 aa, synthetic construct
AVQLVESGGGSVQPGRSMRLSCAASGFTFSNYDMTWVRQAPTKGLEWVASITSDGGSTYSRDSVKGRFTISRDNAKSTLYLQMDSLRSEDTATYYCTTDRGRYLPYYFDYWGQGVMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGSHHHHHH

SEQ ID NO: 567 - protein, 121 aa, synthetic construct; identical to SEQ ID NO: 27

SEQ ID NO: 568 - protein, 218 aa, synthetic construct; identical to SEQ ID NO: 28

SEQ ID NO: 569 - protein, 111 aa, synthetic construct; identical to SEQ ID NO: 29

SEQ ID NO: 570 - protein, 458 aa, synthetic construct; identical to SEQ ID NO: 274

SEQ ID NO: 571 - protein, 119 aa, synthetic construct; identical to SEQ ID NO: 275

SEQ ID NO: 572 - protein, 212 aa, synthetic construct; identical to SEQ ID NO: 276

SEQ ID NO: 573 - protein, 105 aa, synthetic construct; identical to SEQ ID NO: 277

SEQ ID NO: 574 - protein, 456 aa, synthetic construct
EVQLVESGGGLVPPGKSLKLSCSASGFPFSNYGMHWIRQAPGKGLDWVGYISSNSGTIYADAVKGRFTISRDNAKNTLYLLINSLKSEDTAMYYCARGYFDGYYRFWGQGVMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGSHHHHHH

SEQ ID NO: 575 - protein, 117 aa, synthetic construct; identical to SEQ ID NO: 443

SEQ ID NO: 576 - protein, 219 aa, synthetic construct
DIVMTQSPFSLAVSEGDMVTIMCRSSQSLLSSGNQKNYLAWYQQKPGQSPKLLIYHASTRQSGVPDRFIGSGSGTDFTLTISDVQAEDLADYYCLQHYSSPTFGSGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 577 - protein, 112 aa, synthetic construct; identical to SEQ ID NO: 445

SEQ ID NO: 578 - protein, 460 aa, synthetic construct
EVHLVESGGGLVQPGGSLKLSCAASGFTFSNYDMAWVRQAPTRGLEWVASISPGGGKTYYRDSVKGRLTISRNNAENTQYLQIDSLRSEDTATYYCSRLGPAYSGEWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGSHHHHHH

SEQ ID NO: 579 - protein, 121 aa, synthetic construct; identical to SEQ ID NO: 447

SEQ ID NO: 580 - protein, 213 aa, synthetic construct
DTVLTQSPALTVSPGDKITISCRASEGVNTRIHWYQQKSGQQPKLLIYGASNLDSGVPDRFSGSGFGTDFTLTIDPVEASDTATYFCQQSWNVPHTFGGGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 581 - protein, 106 aa, synthetic construct; identical to SEQ ID NO: 449

SEQ ID NO: 582 - protein, 459 aa, synthetic construct
EVQLAESGGGLEQPGRSLKLSCAASGFTFSNYDMAWVRQAPTKGLEWVASIIKSGDTSYYRDSVKGRFTVSRDNAKSTLYLQMDSLRSEDTATYYCARHGVGSYDWFADWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGSHHHHHH

SEQ ID NO: 583 - protein, 120 aa, synthetic construct; identical to SEQ ID NO: 451

SEQ ID NO: 584 - protein, 216 aa, synthetic construct
DIVLTQSPALAVSLGQRATISCRASQSVTISGFNLMHWYQQKPGQQPKLLIYRASNLAFGIPARFSGSGSGTDFTLTINPVQADDFTTYYCQQSRKSRTFGGGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 585 - protein, 109 aa, synthetic construct; identical to SEQ ID NO: 453

SEQ ID NO: 586 - protein, 466 aa, synthetic construct; identical to SEQ ID NO: 542

SEQ ID NO: 587 - protein, 127 aa, synthetic construct; identical to SEQ ID NO: 6

SEQ ID NO: 588 - protein, 214 aa, synthetic construct; identical to SEQ ID NO: 7

SEQ ID NO: 589 - protein, 107 aa, synthetic construct; identical to SEQ ID NO: 8

SEQ ID NO: 590 - protein, 461 aa, synthetic construct; identical to SEQ ID NO: 278

SEQ ID NO: 591 - protein, 122 aa, synthetic construct; identical to SEQ ID NO: 279

SEQ ID NO: 592 - protein, 214 aa, synthetic construct; identical to SEQ ID NO: 7

SEQ ID NO: 593 - protein, 107 aa, synthetic construct; identical to SEQ ID NO: 8

SEQ ID NO: 594 - protein, 463 aa, synthetic construct; identical to SEQ ID NO: 422

SEQ ID NO: 595 - protein, 124 aa, synthetic construct; identical to SEQ ID NO: 19

SEQ ID NO: 596 - protein, 214 aa, synthetic construct; identical to SEQ ID NO: 7

SEQ ID NO: 597 - protein, 107 aa, synthetic construct; identical to SEQ ID NO: 8

SEQ ID NO: 598 - protein, 461 aa, synthetic construct; identical to SEQ ID NO: 282

SEQ ID NO: 599 - protein, 122 aa, synthetic construct; identical to SEQ ID NO: 283

SEQ ID NO: 600 - protein, 214 aa, synthetic construct; identical to SEQ ID NO: 7

SEQ ID NO: 601 - protein, 107 aa, synthetic construct; identical to SEQ ID NO: 8

SEQ ID NO: 602 - protein, 450 aa, synthetic construct
EVQLVESGGGLVQPGGSLRLSCAASGFNIKDTYIHWVRQAPGKGLEWVARIYPTNGYTRYADSVKGRETISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 603 - protein, 120 aa, synthetic construct
EVQLVESGGGLVQPGGSLRLSCAASGFNIKDTYIHWVRQAPGKGLEWVARIYPTNGYTRYADSVKGRETISADTSKNTAYLQMNSLRAEDTAVYYCSRWGGDGFYAMDYWGQGTLVTVSS

SEQ ID NO: 604 - protein, 219 aa, synthetic construct; identical to SEQ ID NO: 56

SEQ ID NO: 605 - protein, 112 aa, synthetic construct; identical to SEQ ID NO: 57

SEQ ID NO: 606 - protein, 447 aa, synthetic construct
QVQLVESGGGLVKPGGSLRLSCAASGFTFSNYYMSWVRQAPGKGLEWISYISGRGSTIFYADSVKGRITISRDNAKNSLFLQMNSLRAEDTAVYFCVKDRGGYSPYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 607 - protein, 117 aa, synthetic construct; identical to SEQ ID NO: 415

SEQ ID NO: 608 - protein, 215 aa, synthetic construct; identical to SEQ ID NO: 416

SEQ ID NO: 609 - protein, 108 aa, synthetic construct; identical to SEQ ID NO: 417

SEQ ID NO: 610 - protein, 446 aa, synthetic construct
EVQLVESGGGLVQPGGSLRLSCAASGYSITNDYAWNWVRQAPGKGLEWVGYISYSGYTTYNPSLKSRFTISRDTSKNTLYLQMNSLRAEDTAVYYCARWTSGLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 611 - protein, 116 aa, synthetic construct; identical to SEQ ID NO: 507

SEQ ID NO: 612 - protein, 214 aa, synthetic construct; identical to SEQ ID NO: 508

SEQ ID NO: 613 - protein, 107 aa, synthetic construct; identical to SEQ ID NO: 509

SEQ ID NO: 614 - protein, 451 aa, synthetic construct; identical to SEQ ID NO: 14

SEQ ID NO: 615 - protein, 121 aa, synthetic construct; identical to SEQ ID NO: 15

SEQ ID NO: 616 - protein, 214 aa, synthetic construct; identical to SEQ ID NO: 16

SEQ ID NO: 617 - protein, 107 aa, synthetic construct; identical to SEQ ID NO: 17

SEQ ID NO: 618 - protein, 454 aa, synthetic construct; identical to SEQ ID NO: 18

SEQ ID NO: 619 - protein, 124 aa, synthetic construct; identical to SEQ ID NO: 19

SEQ ID NO: 620 - protein, 214 aa, synthetic construct; identical to SEQ ID NO: 7

SEQ ID NO: 621 - protein, 107 aa, synthetic construct; identical to SEQ ID NO: 8

SEQ ID NO: 622 - protein, 443 aa, synthetic construct; identical to SEQ ID NO: 22

SEQ ID NO: 623 - protein, 113 aa, synthetic construct; identical to SEQ ID NO: 23

SEQ ID NO: 624 - protein, 214 aa, synthetic construct; identical to SEQ ID NO: 24

SEQ ID NO: 625 - protein, 107 aa, synthetic construct; identical to SEQ ID NO: 25

SEQ ID NO: 626 - protein, 451 aa, synthetic construct; identical to SEQ ID NO: 26

SEQ ID NO: 627 - protein, 121 aa, synthetic construct; identical to SEQ ID NO: 27

SEQ ID NO: 628 - protein, 218 aa, synthetic construct; identical to SEQ ID NO: 28

SEQ ID NO: 629 - protein, 111 aa, synthetic construct; identical to SEQ ID NO: 29

SEQ ID NO: 630 - protein, 447 aa, synthetic construct
EVQLLESGGGLVQPGGSLRLSCAASGFTFSSYAMSWVRQAPGKGLEWVSAINSQGKSTYYADSVKGRFTISRDNSKNTLYLQMNSLRAEDTAVYYCARWGDEGFDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 631 - protein, 117 aa, synthetic construct; identical to SEQ ID NO: 31

SEQ ID NO: 632 - protein, 224 aa, synthetic construct
EVQLLESGGGLVQPGGSLRLSCAASGFTFSSYAMSWVRQAPGKGLEWVSAINSQGKSTYYADSVKGRFTISRDNSKNTLYLQMNSLRAEDTAVYYCARWGDEGFDIWGQGTLVTVSSRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 633 - protein, 117 aa, synthetic construct; identical to SEQ ID NO: 31

SEQ ID NO: 634 - protein, 454 aa, synthetic construct
QVQLVESGGGVVQPGRSLRLSCAASGFTFSTYGMHWVRQAPGKGLEWVAVIWDDGSYKYYGDSVKGRFTISRDNSKNTLYLQMNSLRAEDTAVYYCARDGITMVRGVMKDYFDYWGQGTLVTVSSAKTTPPSVYPLAPGSAAQTNSMVTLGCLVKGYFPEPVTVTWNSGSLSSGVHTFPAVLQSDLYTLSSSVTVPSSPRPSETVTCNVAHPASSTKVDKKIVPRDCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 635 - protein, 125 aa, synthetic construct [sequence omitted]

SEQ ID NO: 636 - protein, 214 aa, synthetic construct
AIQLTQSPSSLSASVGDRVTITCRASQDISSALVWYQQKPGKAPKLLIYDASSLESGVPSRFSGSESGTDFTLTISSLQPEDFATYYCQQFNSYPLTFGGGTKVEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 637 - protein, 107 aa, synthetic construct [sequence omitted]

SEQ ID NO: 638 - protein, 449 aa, synthetic construct
QVQLVQSGSELKKPGASVKISCKASGYDFTNYAMNWVRQAPGHGLEWMGWINANTGDPTYAQGFTGRFVFSLDTSVSTAYLQISSLKAEDSAVYYCTRERFLEWLHFDYWGQGTLVTVSSAKTTPPSVYPLAPGSAAQTNSMVTLGCLVKGYFPEPVTVTWNSGSLSSGVHTFPAVLQSDLYTLSSSVTVPSSPRPSETVTCNVAHPASSTKVDKKIVPRDCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 639 - protein, 120 aa, synthetic construct
QVQLVQSGSELKKPGASVKISCKASGYDFTNYAMNWVRQAPGHGLEWMGWINANTGDPTYAQGFTGRFVFSLDTSVSTAYLQISSLKAEDSAVYYCTRERFLEWLHFDYWGQGTLVTVSS

SEQ ID NO: 640 - protein, 214 aa, synthetic construct
DIQMTQSPSSLSASVGDRVTITCRASQSISSYLNWYQQKPGKAPKLLIYAASSLQSGVPSRFSGSGSGTDFTLTISSLQPEDFATYYCQQSYSTPPTFGQGTKVEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 641 - protein, 107 aa, synthetic construct; identical to SEQ ID NO: 8

SEQ ID NO: 642 - protein, 354 aa, synthetic construct
QVQLQESGGGSVQAGGSLKLSCAASGRSFSTYAMGWFRQAPGQDREFVATISWTDSTDYADSVKGRFTISRDNAKNTGYLQMNSLKPEDTAVYYCAADRWASSRRNVDYDYWGQGTQVTVSSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 643 - protein, 122 aa, synthetic construct
QVQLQESGGGSVQAGGSLKLSCAASGRSFSTYAMGWFRQAPGQDREFVATISWTDSTDYADSVKGRFTISRDNAKNTGYLQMNSLKPEDTAVYYCAADRWASSRRNVDYDYWGQGTQVTVSS

SEQ ID NO: 644 - protein, 356 aa, synthetic construct
QVKLVESGGGVVRPGGSLTLSCAASGRTSRSYGMGWFRQAPGKEREFVSGISWRGDSTGYADSVKGRFTISRDNAKNSLYLQMNSLRAEDTALYYCAAAAGSAWYGTLYEYDYWGQGTLVTVSSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 645 - protein, 124 aa, synthetic construct
QVKLVESGGGVVRPGGSLTLSCAASGRTSRSYGMGWFRQAPGKEREFVSGISWRGDSTGYADSVKGRFTISRDNAKNSLYLQMNSLRAEDTALYYCAAAAGSAWYGTLYEYDYWGQGTLVTVSS

SEQ ID NO: 646 - protein, 448 aa, synthetic construct
QVQLQESGPGLVKPSETLSLTCTVSGGSVSSGDYYWTWIRQSPGKGLEWIGHIYYSGNTNYNPSLKSRLTISIDTSKTQFSLKLSSVTAADTAIYYCVRDRVTGAFDIWGQGTMVTVSSAKTTPPSVYPLAPGSAAQTNSMVTLGCLVKGYFPEPVTVTWNSGSLSSGVHTFPAVLQSDLYTLSSSVTVPSSPRPSETVTCNVAHPASSTKVDKKIVPRDCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 647 - protein, 119 aa, synthetic construct [sequence omitted]

SEQ ID NO: 648 - protein, 214 aa, synthetic construct
DIQMTQSPSSLSASVGDRVTITCQASQDISNYLNWYQQKPGKAPKLLIYDASNLETGVPSRFSGSGSGTDFTFTISSLQPEDIATYFCQHFDHLPLAFGGGTKVEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 649 - protein, 107 aa, synthetic construct [sequence omitted]

SEQ ID NO: 650 - protein, 356 aa, synthetic construct
QVKLVESGGGVVRPGGSLTLSCAASGRTSRSYGMGWFRQAPGKEREFVSGISWRGDSTGYADSVKGRFTISRDNAKNSLYLQMNSLRAEDTALYYCAAAAGSAWYGTLYEYDYWGQGTLVTVSSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 651 - protein, 124 aa, synthetic construct; identical to SEQ ID NO: 645

SEQ ID NO: 652 - protein, 356 aa, synthetic construct
QVKLVESGGGVVRPGGSLTLSCAASGRTSRSYGMGWFRQAPGKEREFVSGISWRGDSTGYADSVKGRFTISRDNAKNSLYLQMNSLRAEDTALYYCAAAAGSAWYGTLYEYDYWGQGTLVTVSSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 653 - protein, 124 aa, synthetic construct; identical to SEQ ID NO: 645

SEQ ID NO: 654 - protein, 356 aa, synthetic construct
QVKLVESGGGVVRPGGSLTLSCAASGRTSRSYGMGWFRQAPGKEREFVSGISWRGDSTGYADSVKGRFTISRDNAKNSLYLQMNSLRAEDTALYYCAAAAGSAWYGTLYEYDYWGQGTLVTVSSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 655 - protein, 124 aa, synthetic construct; identical to SEQ ID NO: 645

SEQ ID NO: 656 - protein, 449 aa, synthetic construct
QVTLRESGPALVKPTQTLTLTCTFSGFSLSTSGMSVGWIRQPPGKALEWLADIWWDDKKDYNPSLKSRLTISKDTSKNQVVLKVTNMDPADTATYYCARSMITNWYFDVWGAGTTVTVSSAKTTPPSVYPLAPGSAAQTNSMVTLGCLVKGYFPEPVTVTWNSGSLSSGVHTFPAVLQSDLYTLSSSVTVPSSPRPSETVTCNVAHPASSTKVDKKIVPRDCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 657 - protein, 120 aa, synthetic construct; identical to SEQ ID NO: 10

SEQ ID NO: 658 - protein, 213 aa, synthetic construct
DIQMTQSPSTLSASVGDRVTITCKCQLSVGYMHWYQQKPGKAPKLLIYDTSKLASGVPSRFSGSGSGTEFTLTISSLQPDDFATYYCFQGSGYPFTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 659 - protein, 106 aa, synthetic construct; identical to SEQ ID NO: 12

SEQ ID NO: 660 - protein, 459 aa, synthetic construct; identical to SEQ ID NO: 9

SEQ ID NO: 661 - protein, 120 aa, synthetic construct; identical to SEQ ID NO: 10

SEQ ID NO: 662 - protein, 213 aa, synthetic construct; identical to SEQ ID NO: 11

SEQ ID NO: 663 - protein, 106 aa, synthetic construct; identical to SEQ ID NO: 12

SEQ ID NO: 664 - protein, 354 aa, synthetic construct; identical to SEQ ID NO: 642

SEQ ID NO: 665 - protein, 122 aa, synthetic construct; identical to SEQ ID NO: 643

SEQ ID NO: 666 - protein, 448 aa, synthetic construct; identical to SEQ ID NO: 302

SEQ ID NO: 667 - protein, 118 aa, synthetic construct; identical to SEQ ID NO: 303

SEQ ID NO: 668 - protein, 213 aa, synthetic construct; identical to SEQ ID NO: 304

SEQ ID NO: 669 - protein, 106 aa, synthetic construct; identical to SEQ ID NO: 305

SEQ ID NO: 670 - protein, 354 aa, synthetic construct; identical to SEQ ID NO: 642

SEQ ID NO: 671 - protein, 122 aa, synthetic construct; identical to SEQ ID NO: 643

SEQ ID NO: 672 - protein, 451 aa, synthetic construct; identical to SEQ ID NO: 306

SEQ ID NO: 673 - protein, 121 aa, synthetic construct [sequence omitted]

SEQ ID NO: 674 - protein, 214 aa, synthetic construct; identical to SEQ ID NO: 308

SEQ ID NO: 675 - protein, 107 aa, synthetic construct [sequence omitted]

SEQ ID NO: 676 - protein, 354 aa, synthetic construct; identical to SEQ ID NO: 642

SEQ ID NO: 677 - protein, 122 aa, synthetic construct; identical to SEQ ID NO: 643

SEQ ID NO: 678 - protein, 446 aa, synthetic construct; identical to SEQ ID NO: 242

SEQ ID NO: 679 - protein, 116 aa, synthetic construct; identical to SEQ ID NO: 243

SEQ ID NO: 680 - protein, 214 aa, synthetic construct; identical to SEQ ID NO: 244

SEQ ID NO: 681 - protein, 107 aa, synthetic construct; identical to SEQ ID NO: 245

SEQ ID NO: 682 - protein, 354 aa, synthetic construct; identical to SEQ ID NO: 642

SEQ ID NO: 683 - protein, 122 aa, synthetic construct; identical to SEQ ID NO: 643

SEQ ID NO: 684 - protein, 447 aa, synthetic construct
QIQLVQSGPELKKPGETVKISCKASGYTFTNYGMNWVRQAPGKGLKWVGWINTNTGEPTYADDFKGRFAFSLETSASTAYLQISNLKNEDMSTYFCAGSHDYSFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 685 - protein, 117 aa, synthetic construct
QIQLVQSGPELKKPGETVKISCKASGYTFTNYGMNWVRQAPGKGLKWVGWINTNTGEPTYADDFKGRFAFSLETSASTAYLQISNLKNEDMSTYFCAGSHDYSFAYWGQGTLVTVSA

SEQ ID NO: 686 - protein, 216 aa, synthetic construct
QAVVTQESALTTSPGETVTFTCRSSTGAVTTSNYANWVQEKPDHLFTGLIGGTNNRAPGVPARFSGSLIGDKAALTITGAQTEDEAIYFCALWYSNHWVFGGGTKLTVLRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 687 - protein, 109 aa, synthetic construct
QAVVTQESALTTSPGETVTFTCRSSTGAVTTSNYANWVQEKPDHLFTGLIGGTNNRAPGVPARFSGSLIGDKAALTITGAQTEDEAIYFCALWYSNHWVFGGGTKLTVL

SEQ ID NO: 688 - protein, 444 aa, synthetic construct
QVQLQQSGTELMKPGASVKISCKATGYTFSSYWIEWVKQRPGHGLEWIGEILPGSGSTNYNEKFKGKATFTVDTSSNTAYMQLSSLTSEDSAVYYCARIIRDFWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 689 - protein, 114 aa, synthetic construct
QVQLQQSGTELMKPGASVKISCKATGYTFSSYWIEWVKQRPGHGLEWIGEILPGSGSTNYNEKFKGKATFTVDTSSNTAYMQLSSLTSEDSAVYYCARIIRDFWGQGTTLTVSS

SEQ ID NO: 690 - protein, 214 aa, synthetic construct
DIQMTQSSSSFSVSLGDRVTITCKASEDIYNRLAWFQQKPGNAPRLLISGATSLETGVPSRFSGSRSGEDYTLIITSLQTEDVATYYCQQFWTTPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 691 - protein, 107 aa, synthetic construct
DIQMTQSSSSFSVSLGDRVTITCKASEDIYNRLAWFQQKPGNAPRLLISGATSLETGVPSRFSGSRSGEDYTLIITSLQTEDVATYYCQQFWTTPPTFGGGTKLEIK

SEQ ID NO: 692 - protein, 443 aa, synthetic construct
QVQLQQSGAELMKPGASVKISCKATGYTFSRYWIEWVKQRPGHGLEWIGEILPGSGSTNYNEKFKGKATFTADTSSNTAYMQLTSLTSEDSAVYFCERRGAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 693 - protein, 113 aa, synthetic construct
QVQLQQSGAELMKPGASVKISCKATGYTFSRYWIEWVKQRPGHGLEWIGEILPGSGSTNYNEKFKGKATFTADTSSNTAYMQLTSLTSEDSAVYFCERRGAYWGQGTLVTVSA

SEQ ID NO: 694 - protein, 214 aa, synthetic construct
DIQMTQSSSSFSVSLGDRVTITCKASEDIYNRLAWYQQKPGNAPRLLISGATSLETGVPSRFSGSGSGKDYTLSITSLQTEDVATYHCQQNWSTPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 695 - protein, 107 aa, synthetic construct
DIQMTQSSSSFSVSLGDRVTITCKASEDIYNRLAWYQQKPGNAPRLLISGATSLETGVPSRFSGSGSGKDYTLSITSLQTEDVATYHCQQNWSTPPTFGGGTKLEIK

SEQ ID NO: 696 - protein, 452 aa, synthetic construct
QVQLQQPGAELVRPGASVKLSCKASGYTFTSYWMNWVKQRPGQGLEWIGMIDPSDSETHYSQMFKDKATLTVDQSSNTAYMHLSSLTSEDSAVYYCAREGYYFDGTRGIAYWGQGTLVTVSVASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 697 - protein, 122 aa, synthetic construct
QVQLQQPGAELVRPGASVKLSCKASGYTFTSYWMNWVKQRPGQGLEWIGMIDPSDSETHYSQMFKDKATLTVDQSSNTAYMHLSSLTSEDSAVYYCAREGYYFDGTRGIAYWGQGTLVTVSV

SEQ ID NO: 698 - protein, 214 aa, synthetic construct
DIQMTQSTSSLSASLGDRVTISCRASQDISNYLNWYQQKPDGTVKPLIYYTSRLHSGVPSRFSGSGSGTDYSLTISNLDQEDIATYFCQQGNTLPFTFGSGTKLAIERTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 699 - protein, 107 aa, synthetic construct
DIQMTQSTSSLSASLGDRVTISCRASQDISNYLNWYQQKPDGTVKPLIYYTSRLHSGVPSRFSGSGSGTDYSLTISNLDQEDIATYFCQQGNTLPFTFGSGTKLAIE

SEQ ID NO: 700 - protein, 446 aa, synthetic construct
QVQLQQSGAEVMKPGASVKLSCKATGYTFTGYWIEWVKERPGHGLEWIGEILPGSGSTNYNEKFKGKATFTADPSSNTAYMQLSSLTTEDSAIYYCARDYGSFGYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 701 - protein, 116 aa, synthetic construct
QVQLQQSGAEVMKPGASVKLSCKATGYTFTGYWIEWVKERPGHGLEWIGEILPGSGSTNYNEKFKGKATFTADPSSNTAYMQLSSLTTEDSAIYYCARDYGSFGYWGQGTLVTVSA

SEQ ID NO: 702 - protein, 214 aa, synthetic construct
DIVMTQSQKFMSTSVGDRVSVTCKASQNVGTNVAWFQQKPGQSPKLLIYSASYRYSGVPDRFTGSGSGTDFTLTISNVQSEDLAEFFCQQYNTYPLTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 703 - protein, 107 aa, synthetic construct
DIVMTQSQKFMSTSVGDRVSVTCKASQNVGTNVAWFQQKPGQSPKLLIYSASYRYSGVPDRFTGSGSGTDFTLTISNVQSEDLAEFFCQQYNTYPLTFGAGTKLELK

SEQ ID NO: 704 - protein, 446 aa, synthetic construct
EVQLQQSGAELVRPGASVKLSCTASGFNIKDDYMHWMKQRPEQGLEWLGWIDPEIGATEYASKFQGKATMTADTSSNTAYLQLSSLTSEDTAVYYCVDDRRGMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 705 - protein, 116 aa, synthetic construct
EVQLQQSGAELVRPGASVKLSCTASGFNIKDDYMHWMKQRPEQGLEWLGWIDPEIGATEYASKFQGKATMTADTSSNTAYLQLSSLTSEDTAVYYCVDDRRGMDYWGQGTSVTVSS

SEQ ID NO: 706 - protein, 219 aa, synthetic construct
DIVMTQDAPSIPVTPGESVSISCRSSKSLLHSNGNTYLYWFLQRPGQSPQLLIYRMSNLASGVPDRFSGSGSGTAFTLRISRVEAEDVGVYYCMQHLEYPFTFGSGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 707 - protein, 112 aa, synthetic construct
DIVMTQDAPSIPVTPGESVSISCRSSKSLLHSNGNTYLYWFLQRPGQSPQLLIYRMSNLASGVPDRFSGSGSGTAFTLRISRVEAEDVGVYYCMQHLEYPFTFGSGTKLEIK

SEQ ID NO: 708 - protein, 449 aa, synthetic construct
QVSLKESGPGILQPSQTLSLTCSFSGFSLSTSGMAVGWIRQPSGRGLEWLANIWWDDSQHYNAALKSRLTISKDTSKNQVFLKIASVDTADTATYYCARSNWGRYFDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 709 - protein, 119 aa, synthetic construct
QVSLKESGPGILQPSQTLSLTCSFSGFSLSTSGMAVGWIRQPSGRGLEWLANIWWDDSQHYNAALKSRLTISKDTSKNQVFLKIASVDTADTATYYCARSNWGRYFDYWGQGTTLTVSS

SEQ ID NO: 710 - protein, 219 aa, synthetic construct
DIVMSQSPSSLAVSVGEEVTMSCKSSQSLLYSTNQKNYLAWYQQKPGQSPKLLIYWASTRESGVPDRFTGSGSGTDFTLTISSVKAEDLAVYYCQQYYDYYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 711 - protein, 112 aa, synthetic construct
DIVMSQSPSSLAVSVGEEVTMSCKSSQSLLYSTNQKNYLAWYQQKPGQSPKLLIYWASTRESGVPDRFTGSGSGTDFTLTISSVKAEDLAVYYCQQYYDYYTFGGGTKLEIK

SEQ ID NO: 712 - protein, 442 aa, synthetic construct
QVQLQQPGAELVKPGASVKLSCKASGYTFTTYYIYWVKQRPGQGLEWIGGINPRNGGTNFNEKFKTRATLTVDKSSSTAYMQLSSLTSEDSAVYYCTRTFYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 713 - protein, 112 aa, synthetic construct
QVQLQQPGAELVKPGASVKLSCKASGYTFTTYYIYWVKQRPGQGLEWIGGINPRNGGTNFNEKFKTRATLTVDKSSSTAYMQLSSLTSEDSAVYYCTRTFYWGQGTTLTVSS

SEQ ID NO: 714 - protein, 214 aa, synthetic construct
DIQMTQTTSSLSASLGDRVTISCSASQGIGNYLNWYQQKPDGTVKLLIYYTSSLNSGVPSRFSGSGSGTDYSLTISNLEPEDIATYFCQQYSKLPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 715 - protein, 107 aa, synthetic construct
DIQMTQTTSSLSASLGDRVTISCSASQGIGNYLNWYQQKPDGTVKLLIYYTSSLNSGVPSRFSGSGSGTDYSLTISNLEPEDIATYFCQQYSKLPYTFGGGTKLEIK

SEQ ID NO: 716 - protein, 445 aa, synthetic construct
EVQLQQSGAELVRPGASVKLSCTASGFNIIVDYLHWVRQRPEQGLEWIGWIDPEIGSTEYASKFQGKATMTADTSSNTAYLQLSSLTSEDTAVYYCIIDGTMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 717 - protein, 115 aa, synthetic construct
EVQLQQSGAELVRPGASVKLSCTASGFNIIVDYLHWVRQRPEQGLEWIGWIDPEIGSTEYASKFQGKATMTADTSSNTAYLQLSSLTSEDTAVYYCIIDGTMDYWGQGTSVTVSS

SEQ ID NO: 718 - protein, 219 aa, synthetic construct
DIVMTQAAPSVPVTPGESVSISCRSSKSLLHSNGNTYLYWFLQRPGQSPQVLIYRMSNLASGVPDRFSGSGSGTAFTLRISRVEAEDVGVYYCMQHLEYPFTFGSGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 719 - protein, 112 aa, synthetic construct
DIVMTQAAPSVPVTPGESVSISCRSSKSLLHSNGNTYLYWFLQRPGQSPQVLIYRMSNLASGVPDRFSGSGSGTAFTLRISRVEAEDVGVYYCMQHLEYPFTFGSGTKLEIK

SEQ ID NO: 720 - protein, 447 aa, synthetic construct
EVQLQQSGAELVRPGASVKLSCTASGFNIKDDYIHWVKQRPEQGLEWIGWIDPENGDTKYASKFPGKATMTADTSSNTAYLQLSSLTSEDTAVYYCTASRTTALDYWGPGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 721 - protein, 117 aa, synthetic construct
EVQLQQSGAELVRPGASVKLSCTASGFNIKDDYIHWVKQRPEQGLEWIGWIDPENGDTKYASKFPGKATMTADTSSNTAYLQLSSLTSEDTAVYYCTASRTTALDYWGPGTTLTVSS

SEQ ID NO: 722 - protein, 214 aa, synthetic construct
DIVMTQSQKFMSTSVGDRVSITCKASQSVRPAVAWYQQKPGQSPKALIYLASNRHTGVPDRFTGSGSGTDFTLTISNVQSEDLADYFCLQHWNYPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 723 - protein, 107 aa, synthetic construct
DIVMTQSQKFMSTSVGDRVSITCKASQSVRPAVAWYQQKPGQSPKALIYLASNRHTGVPDRFTGSGSGTDFTLTISNVQSEDLADYFCLQHWNYPYTFGGGTKLEIK

SEQ ID NO: 724 - protein, 356 aa, synthetic construct; identical to SEQ ID NO: 654

SEQ ID NO: 725 - protein, 124 aa, synthetic construct; identical to SEQ ID NO: 645

SEQ ID NO: 726 - protein, 5 aa, synthetic construct
EPKSC

SEQ ID NO: 727 - protein, 5 aa, synthetic construct
GGGGS